(19) United States
(12) Patent Application Publication
(10) Pub. No.: US 2006/0257894 A1
(43) Pub. Date: Nov. 16, 2006

(54) Molecular typing of listeria monocytogenes, hybridization supports and kits for said molecular typing

(76) Inventors: Michel Doumith, Paris (FR); Carmen Buchrieser, Paris (FR); Philippe Glaser, Paris (FR); Paul Martin, Paris (FR); Christine Jacquet, Le Kremlin Blcetre (FR); Patricia Garrido, Madrid (ES); Christophe Rusniok, Leplessis Robinson (FR); Frederik Kunst, Ivry Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/357,421

(22) Filed: Feb. 21, 2006

Related U.S. Application Data
(63) Continuation-in-part of application No. 11/154,929, filed on Jun. 17, 2005, now abandoned.
Application No. 11/154,929 is a continuation of application No. 10/942,074, filed on Sep. 16, 2004, now abandoned.
(60) Provisional application No. 60/502,935, filed on Sep. 16, 2003.

(51) Int. Cl. C12Q 1/68; C07H 21/04

(52) U.S. Cl. 435/6; 536/23.7

(57) ABSTRACT

This invention provides 163 genes that are unique to the L. monocytogenes serovar 4b strain, CLIP80459, compared with L. monocytogenes EGDe strain. These genes are the basis for isolated nucleic acids, hybridization supports, and kits comprising a nucleotide sequence that is unique to a serovar 4b strain of L. monocytogenes. The invention also provides methods for identifying a L. monocytogenes in a sample, methods for identifying the lineage of a L. monocytogenes in a sample, and methods for sub-lineage typing of a L. monocytogenes in a sample. The invention also provides hybridization supports comprising a L. monocytogenes lineage-specific nucleic acid sequence, hybridization supports comprising a L. monocytogenes sub-lineage-specific nucleic acid sequence combination, and kits comprising a nucleic acid or hybridization support of the invention. Additionally, the invention provides specific macroarrays, methods, and kits for use in serotyping L. monocytogenes.

This application is a continuation-in-part of application Ser. No. 11/154,929, filed Jun. 17, 2005, which is a continuation of application Ser. No. 10/942,074, filed Sep. 16, 2004, which claims priority to Provisional Application No. 60/502,935, filed Sep. 16, 2003. The entire disclosure of each of these priority applications is hereby incorporated herein by reference for all purposes.

### BACKGROUND OF THE INVENTION

The sequencing of complete microbial genomes has revealed insights into the genetic structure of a number of bacterial species. However, it has become evident that the genome sequence of one strain is not entirely representative for other members of the species, and that the broad spectrum of physiological and virulence properties of bacterial pathogens mirrors the existence of different subsets of genes enabling different lifestyles. In addition to whole genome comparisons (1, 2), micro- and macro-array techniques are extensively used to study inter- and intra-species diversity of bacterial pathogens (3, 4). These studies suggest that, although considerable diversity is present among different bacterial isolates of the same species, clonal expansion of highly virulent sub-populations of a bacterial pathogen may exist.

L. monocytogenes, an intracellular pathogen, is the causative agent of serious epidemic and sporadic food-borne listeriosis. The clinical features of listeriosis include meningitis, meningo-encephalitis, septicemia, abortion, perinatal infections and gastroenteritis (5). Although rare when compared to other food-borne diseases, a significant feature of listeriosis is the high lethality rate (about 30%), which makes L. monocytogenes an important human pathogen. L. monocytogenes has the capacity to adapt and survive in extreme conditions, allowing it to ubiquitously exist in the environment and to survive and proliferate under conditions that exist within the food chain.

L. monocytogenes strains are commonly differentiated by the serotyping method. Serotyping of L. monocytogenes is based on the variation of fifteen somatic and five flagellar antigens, combinations of which have defined 13 different serovars of the organism (Seeliger and Höhne,1979). Interestingly, not all strains of L. monocytogenes are equally capable of causing disease in humans. Isolates from four (1/2a; 1/2c; 1/2b; 4b) of the 13 serovars (sv) identified within this species are responsible for over 98% of the human listeriosis cases reported (6). A number of different typing and population genetic studies suggested that different genetic divisions/lineages exist within the species L. monocytogenes, and correlate with serovars (7-10).

Specifically, lineage I comprises, sv 1/2a, 1/2c, and 3c, lineage II comprises sv 4b, 1/2b and 3b, and lineage III comprises sv 4a and 4c. Genetic analyses using multilocus sequence typing of virulence-associated genes, RFLP analysis and ribotyping suggested that epidemic strains are mostly found in lineage 11 and sporadic strains in lineage I and II, while lineage III strains are extremely rare and mostly animal pathogens (11, 12). However, these methods are unable to further characterize the genetic basis for this observed variability.

Numerous genotyping methods, based on the detection of DNA polymorphism, provide sensitive strain discrimination and a high level of standardization and reproducibility were developed to link a human case of listeriosis to suspect food items. The most widely used molecular methods providing highly discriminating results for L. monocytogenes include ribotyping and pulsed field gel electrophoresis (Brosch et al. 1996; Bruce et al. 1995). However, groupings based on these methods are still often compared to groupings on serotype since this nomenclature is widely used and established. Furthermore, in spite of the highly reproducible and discriminating power indispensable to tracking the contamination source in foods, ribotyping and pulsed field gel electrophoresis methods are costly or laborious and time consuming. Thus, in listeriosis outbreak investigations, subgrouping by serotyping has proven utility as a first subtyping method for practical purposes and time savings in reducing the number of suspected isolates.

However, conventional serological assays using slide agglutination to serotype L. monocytogenes isolates are complex, and require up to three days to be completed. Additionally, this assay relies on visual acuity and judgment, which may be a substantial source of variability when comparing results between individuals in a laboratory and different laboratories. In addition, the assay is difficult to automate, so that it is difficult to use this method to characterize a large number of isolates.

Recently, Zhang et al., Journal of Bacteriology, Vol.185, No.18, pp. 5573-5584 (2003), reported probing 44 strains with a DNA microarray constructed from strain 10403s.

Meanwhile, the complete genome sequences of Listeria monocytogenes strain EGDe and Listeria innocua strain CLIP11262 were determined (13). The genome sequence of Listeria monocytogenes strain EGDe (deposited at the CNCM on Apr. 11, 2000, under Accession No. I-2440) is described in FR 00/04629, filed Apr. 11, 2000. The genome sequence of Listeria innocua strain CLIP11262 (deposited at the CNCM on Oct. 2, 2000, under Accession Nos. I-2565 and I-2566) is described in FR 00/12,697, filed Oct. 4, 2000. The comparison of these sequences revealed 10.5% and 14% specific sequences for each isolate, respectively (13). L. monocytogenes strain EGDe is of sv 1/2a and belongs to lineage I. However, all major food-borne outbreaks of listeriosis, as well as the majority of sporadic cases, have been caused by serovar 4b strains, which belong to lineage II. This suggests that strains of this sv may possess unique virulence properties.

Accordingly, there exists a need in the art to provide the sequence of the genome of a serovar 4b strain of L. monocytogenes. In particular, there is a need to identify genes or markers of a serovar 4b strain which are unique relative to strain EGDe. There also exists a need to provide strain-specific genetic markers of L. monocytogenes strains and methods of using strain-specific genetic markers of L. monocytogenes strains to characterize L. monocytogenes isolates.

### SUMMARY OF THE INVENTION

The inventors have met this need by providing 163 genes that are unique to the L. monocytogenes serovar 4b strain, CLIP80459, compared with L. monocytogenes EGDe strain. These genes are the basis for isolated nucleic acids, hybridization supports, and kits comprising a nucleotide sequence that is unique to the L. monocytogenes serovar 4b strain, CLIP80459, compared with L. monocytogenes EGDe strain.

The invention also provides methods for identifying a L. monocytogenes in a sample, methods for identifying the lineage of a L. monocytogenes in a sample, and methods for sub-lineage typing of a L. monocytogenes in a sample.

The invention also provides hybridization supports comprising a L. monocytogenes lineage-specific nucleic acid sequence, hybridization supports comprising a L. monocytogenes sub-lineage-specific nucleic acid sequence combination, and kits comprising a nucleic acid or hybridization support of the invention.

In a first embodiment, the invention provides an isolated nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

In another embodiment, the invention provides a hybridization support comprising a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

In another embodiment, the invention provides a vector comprising a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

The invention also provides a recombinant cell comprising a vector comprising a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

In a further embodiment, the invention provides an isolated polypeptide comprising a polypeptide sequence selected from SEQ ID NOS: 164-326; or a fragment or variant of polypeptide sequence selected from SEQ ID NOS: 164-326.

In another embodiment the invention provides an isolated antibody that specifically binds to a polypeptide comprising a polypeptide sequence selected from SEQ ID NOS: 164-326; or a fragment or variant of polypeptide sequence selected from SEQ ID NOS: 164-326. In a preferred embodiment, the antibody is a monoclonal antibody.

In further embodiments, the invention provides a hybridization support comprising an isolated polypeptide comprising a polypeptide sequence selected from SEQ ID NOS: 164-326; or a fragment or variant of polypeptide sequence selected from SEQ ID NOS: 164-326. The invention also provides a hybridization support comprising an antibody that specifically binds to a polypeptide comprising a polypeptide sequence selected from SEQ ID NOS: 164-326; or a fragment or variant of polypeptide sequence selected from SEQ ID NOS: 164-326.

Another embodiment of the invention provides a method for distinguishing between L. monocytogenes serovar 4b strain CLIP80459 and L. monocytogenes strain EGDe in a sample, wherein the method comprises detecting a nucleic acid in the sample, the nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

The invention also provides a method for distinguishing between L. monocytogenes serovar 4b strain CLIP80459 and L. monocytogenes strain EGDe in a sample. The method comprises (a) bringing the sample into contact with the hybridization support comprising a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5; and (b) detecting a specific hybrid between the hybridization support and a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

The invention further provides a method for identifying a L. monocytogenes in a sample. The method comprises detecting a L. monocytogenes-specific nucleic acid sequence in the sample. In some embodiments the L. monocytogenes-specific nucleic acid sequence is Imo0082, Imo0083, plcA, actA, plcB, Imo206, inlA, inlB, Imo0440, Imo0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821; or a fragment or variant of Imo0082, Imo0083, plcA, actA, plcB, Imo206, inlA, inlB, Imo0440, Imo 0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821. In further embodiments the L. monocytogenes-specific nucleic acid sequence is inlA, inlB, Imo2085, Imo1413, Imo0463, Imo0460, Imo0550, Imo0160, Imo0333, inlE, inlH, Imo2821, Imo2027, inlC, Imo0835, Imo1666, inIF, Imo320, Imo0842, Imo0801, inlG, Imo1115, Imo2576, Imo0171, Imo2026, ORF2568, ORF1761, or ORF0029; or a fragment or variant of inlA, inlB, Imo2085, Imo1413, Imo0463, Imo0460, Imo0550, Imo0160, Imo0333, inlE, inlH, Imo2821, Imo2027, inlC, Imo0835, Imo1666, inlF, Imo320, Imo0842, Imo0801, inlG, Imo115, Imo2576, Imo0171, Imo2026, ORF2568, ORF1761, or ORF0029.

The invention further provides a method for identifying the lineage of a L. monocytogenes in a sample. The method comprises detecting the absence or the presence of a L. monocytogenes lineage-specific nucleic acid sequence in the sample. In some embodiments the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

In further embodiments the L. monocytogenes lineage is lineage I and the L. monocytogenes lineage-specific nucleic acid sequence is a L. monocytogenes lineage I-specific nucleic acid sequence. In some embodiments the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

In further embodiments the L. monocytogenes lineage is lineage II and the L. monocytogenes lineage-specific nucleic acid sequence is a L. monocytogenes lineage II-specific nucleic acid sequence. In some embodiments the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

In further embodiments the L. monocytogenes lineage is lineage III and the L. monocytogenes lineage III-specific nucleic acid sequence is a L. monocytogenes lineage III-specific nucleic acid sequence. In some embodiments the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1666, Imo0835, Imo0036, Imo0037, Imo0038, Imo0039, Imo0040, Imo0041, Imo94, Imo832, Imo1441, Imo1451, or Imo2671.

The invention also provides a method for sub-lineage typing of a L. monocytogenes in a sample. The method comprises determining a L. monocytogenes sub-lineage-specific nucleic acid sequence combination for the L. monocytogenes in the sample.

In some embodiments, the determining a L. monocytogenes sub-lineage-specific nucleic acid sequence combination for the L. monocytogenes in the sample comprises hybridizing a probe or probe combination comprising a L. monocytogenes sub-lineage-specific nucleic acid sequence or sequences to L. monocytogenes genomic DNA obtained from the sample, and detecting a hybridization product comprising the probe or probe combination and a L. monocytogenes genomic DNA sequence.

In some embodiments the determining a L. monocytogenes sub-lineage-specific nucleic acid sequence combination for the L. monocytogenes in the sample comprises hybridizing a primer or primer combination comprising a L. monocytogenes sub-lineage-specific nucleic acid sequence or sequences to L. monocytogenes genomic DNA obtained from the sample; amplifying a L. monocytogenes sub-lineage-specific fragment or fragments with a polymerase; and detecting the L. monocytogenes sub-lineage-specific fragment or fragments.

In some embodiments the L. monocytogenes sub-lineage-specific nucleic acid sequence combination comprises a lineage I-specific nucleic acid sequence; a sublineage I.1- or I.2-specific nucleic acid sequence; a lineage II-specific nucleic acid sequence; and a sublineage II.1- or II.2-specific nucleic acid sequence.

In some embodiments the L. monocytogenes sub-lineage-specific nucleic acid sequence combination comprises a sublineage I.2-specific nucleic acid sequence and a sublineage II.1-specific nucleic acid sequence.

In some embodiments the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

In some embodiments the sublineage I.2-specific nucleic acid sequence is Imo151, Imo466, Imo467, Imo469, Imo470, Imo471, Imo1118 or Imo1119; or a fragment or variant of Imo151, Imo466, Imo467, Imo469, Imo470, Imo471, Imo1118 or Imo1119.

In some embodiments the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

In some embodiments the sublineage II.1-specific nucleic acid sequence is ORF 0799, ORF 2372 or ORF 2110; or a fragment or variant of ORF 0799, ORF 2372 or ORF 2110.

In some embodiments the lineage I-specific nucleic acid sequence is Imo737 or a fragment thereof, the sublineage I.2-specific nucleic acid sequence is Imo1119 or a fragment thereof, the lineage II-specific nucleic acid sequence is ORF 2819 or a fragment or variant thereof, and the sublineage II.1 -specific nucleic acid sequence is ORF 2110 or a fragment thereof.

In some embodiments, the determining a L. monocytogenes sub-lineage-specific nucleic acid sequence combination for the L. monocytogenes in the sample comprises (a) hybridizing a probe combination comprising i) a lineage I-specific nucleic acid sequence; ii) a sublineage I.2-specific nucleic acid sequence; iii) a lineage II-specific nucleic acid sequence; and iv) a sublineage II.1-specific nucleic acid sequence to L. monocytogenes genomic DNA obtained from the sample; and (b) detecting a hybridization product comprising the probe or probe combination and a L. monocytogenes genomic DNA sequence.

In a preferred embodiment the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1 -specific nucleic acid sequence is ORF 2110 or a fragment or variant thereof.

In some embodiments the determining a L. monocytogenes sub-lineage-specific nucleic acid sequence combination for the L. monocytogenes in the sample comprises (a) hybridizing a primer or primer combination comprising i) a lineage I-specific nucleic acid sequence; ii) a sublineage I.2-specific nucleic acid sequence; iii) a lineage II-specific nucleic acid sequence; and iv) a sublineage II.1-specific nucleic acid sequence to L. monocytogenes genomic DNA obtained from the sample; and (b) amplifying a L. monocytogenes sub-lineage-specific fragment or fragments with a polymerase; and (c) detecting the L. monocytogenes sub-lineage-specific fragment or fragments.

In a preferred embodiment the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

The invention also provides a hybridization support comprising a L. monocytogenes-specific nucleic acid sequence. In some embodiments the L. monocytogenes-specific nucleic acid sequence is Imo0082, Imo0083, plcA, actA, plcB, Imo206, inlA, inlB, Imo0440, Imo0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821; or a fragment or variant of Imo0082, Imo0083, plcA, actA, plcB, Imo206, inlA, inlB, Imo0440, Imo 0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821. In some embodiments the L. monocytogenes-specific nucleic acid sequence is inlA, inlB, Imo2085, Imo1413, Imo0463, Imo0460, Imo0550, Imo0160, Imo0333, inlE, inlH, Imo2821, Imo2027, inlC, Imo0835, Imo1666, inlF, Imo320, Imo0842, Imo0801, inlG, Imo1115, Imo2576, Imo0171, Imo2026, ORF2568, ORF1761, or ORF0029; or a fragment or variant of inlA, inlB, Imo2085, Imo1413, Imo0463, Imo0460, Imo0550, Imo0160, Imo0333, inlE, inlH, Imo2821, Imo2027, inlC, Imo0835, Imo1666, inlF, Imo320, Imo0842, Imo0801, inlG, Imo1115, Imo2576, Imo0171, Imo2026, ORF2568, ORF1761, or ORF0029.

The invention also provides a hybridization support comprising a L. monocytogenes lineage-specific nucleic acid sequence. In some embodiments the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

In further embodiments the L. monocytogenes lineage is lineage I and the L. monocytogenes lineage-specific nucleic acid sequence is a L. monocytogenes lineage I-specific nucleic acid sequence. In some embodiments the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973or Imo 1974.

In further embodiments the L. monocytogenes lineage is lineage II and the L. monocytogenes lineage-specific nucleic acid sequence is a L. monocytogenes lineage II-specific nucleic acid sequence. In some embodiments, the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

In further embodiments the L. monocytogenes lineage is lineage III and the L. monocytogenes lineage-specific nucleic acid sequence is a L. monocytogenes lineage III-specific nucleic acid sequence. In some embodiments the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1666, Imo0835, Imo0036, Imo0037, Imo0038, Imo0039, Imo0040, Imo0041, Imo94, Imo832, Imo1441, Imo1451, or Imo2671.

The invention also provides a hybridization support comprising a L. monocytogenes sub-lineage-specific nucleic acid sequence combination.

In some embodiments the L. monocytogenes sub-lineage-specific nucleic acid sequence combination comprises a lineage I-specific nucleic acid sequence; a sublineage I.1- or I.2-specific nucleic acid sequence; a lineage II-specific nucleic acid sequence; and a sublineage II.1- or II.2-specific nucleic acid sequence.

In some embodiments the L. monocytogenes sub-lineage-specific nucleic acid sequence combination comprises a sublineage I.2-specific nucleic acid sequence and a sublineage II.1-specific nucleic acid sequence. In some embodiments the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974. In some embodiments the sublineage I.2-specific nucleic acid sequence is Imo151, Imo466, Imo467, Imo469, Imo470, Imo471, Imo1118 or Imo1119; or a fragment or variant of Imo151, Imo466, Imo467, Imo469, Imo470, Imo471, Imo1118 or Imo1119. In some embodiments the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761. In some embodiments the sublineage II.1-specific nucleic acid sequence is ORF 0799, ORF 2372 or ORF 2110; or a fragment or variant of ORF 0799, ORF 2372 or ORF 2110. In a preferred embodiment, the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

The invention also provides a kit for use in a method for distinguishing between L. monocytogenes serovar 4b strain CLIP80459 and L. monocytogenes strain EGDe in a sample, wherein the kit comprises a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

The invention also provides a kit for use in a method for distinguishing between L. monocytogenes serovar 4b strain CLIP80459 and L. monocytogenes strain EGDe in a sample, wherein the kit comprises a hybridization support comprising a nucleic acid comprising a nucleotide sequence selected from SEQ ID NOS: 1-163; a fragment or variant of a nucleotide sequence selected from SEQ ID NOS: 1-163; a degenerate variant of a nucleotide sequence selected from SEQ ID NOS: 1-163, wherein the degenerate variant encodes a polypeptide sequence selected from SEQ ID NOS: 164-326; or a fragment of a nucleotide sequence selected from SEQ ID NOS: 1-163, wherein the fragment is a forward or reverse primer shown in Table 5.

The invention further provides a kit for use in a method for identifying a L. monocytogenes in a sample, wherein the kit comprises a hybridization support comprising a L. monocytogenes-specific nucleic acid sequence, in packaged form. In some embodiments the L. monocytogenes-specific nucleic acid sequence is Imo0082, Imo0083, plcA, actA, plcB, Imo206, inlA, inlB, Imo0440, Imo0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821; or a fragment or variant of Imo0082, Imo0083, plcA, actA, plcB, Imo206, inlA, inlB, Imo0440, Imo 0638, Imo0733, Imo0833, Imo1133, Imo1135, Imo2732, Imo2733, Imo2734, Imo2736, Imo0017, Imo0094, inlH, inlE, Imo0333, Imo0549, Imo0832, Imo0834, Imo0835, Imo1441, Imo1451, or Imo2821. In some embodiments the L. monocytogenes-specific nucleic acid sequence is inlA, inlB, Imo2085, Imo1413, Imo0463, Imo0460, Imo0550, Imo0160, Imo0333, inlE, inlH, Imo2821, Imo2027, inlC, Imo0835, Imo1666, inlF, Imo320, Imo0842, Imo0801, inlG, Imo1115, Imo2576, Imo0171, Imo2026, ORF2568, ORF1761, or ORF0029; or a fragment or variant of inlA, inlB, Imo2085, Imo1413, Imo0463, Imo0460, Imo0550, Imo0160, Imo0333, inlE, inlH, Imo2821, Imo2027, inlC, Imo0835, Imo1666, inlF, Imo320, Imo0842, Imo0801, inlG, Imo1115, Imo2576, Imo0171, Imo2026, ORF2568, ORF1761, or ORF0029.

The invention further provides a kit for use in a method for identifying the lineage of a L. monocytogenes in a sample, wherein the kit comprises a L. monocytogenes lineage-specific nucleic acid sequence. In some embodiments the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

In further embodiments the L. monocytogenes lineage is lineage I and the L. monocytogenes lineage-specific nucleic acid sequence is a L. monocytogenes lineage I-specific nucleic acid sequence. In some embodiments the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973or Imo 1974.

In further embodiments the L. monocytogenes lineage is lineage II and the L. monocytogenes lineage-specific nucleic acid sequence is a L. monocytogenes lineage II-specific nucleic acid sequence. In some embodiments the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

In further embodiments the L. monocytogenes lineage is lineage III and the L. monocytogenes lineage-specific nucleic acid sequence is a L. monocytogenes lineage III-specific nucleic acid sequence. In some embodiments the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1666, Imo0835, Imo0036, Imo0037, Imo0038, Imo0039, Imo0040, Imo0041, Imo94, Imo832, Imo1441, Imo1451, or Imo2671.

The invention also provides a kit for use in a method for identifying the sub-lineage of a L. monocytogenes in a sample, wherein the kit comprises a L. monocytogenes sub-lineage-specific nucleic acid sequence combination.

In some embodiments the L. monocytogenes sub-lineage-specific nucleic acid sequence combination comprises a lineage I-specific nucleic acid sequence a sublineage I.1- or I.2-specific nucleic acid sequence; a lineage II-specific nucleic acid sequence; and a sublineage II.1- or II.2-specific nucleic acid sequence.

In some embodiments the L. monocytogenes sub-lineage-specific nucleic acid sequence combination comprises a sublineage I.2-specific nucleic acid sequence and a sublineage II.1-specific nucleic acid sequence. In some embodiments the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974. In some embodiments the sublineage I.2-specific nucleic acid sequence is Imo151, Imo466, Imo467, Imo469, Imo470, Imo471, Imo1118 or Imo1119; or a fragment or variant of Imo151, Imo466, Imo467, Imo469, Imo470, Imo471, Imo1118 or Imo1119. In some embodiments the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761. In some embodiments the sublineage II.1-specific nucleic acid sequence is ORF 0799, ORF 2372 or ORF 2110; or a fragment of ORF 0799, ORF 2372 or ORF 2110. In a preferred embodiment the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

The invention also provides a hybridization support comprising a probe specific for L. monocytogenes EGDe relative to L. innocua CLIP11262, a probe specific for L. innocua CLIP11262 relative to L. monocytogenes EGDe, and a probe present only in L. monocytogenes CLIP80459. In a preferred embodiment the hybridization support comprises 262 probes specific for L. monocytogenes EGDe relative to L. innocua CLIP11262 and all virulence genes, 94 probes specific for L. innocua CLIP11262 relative to L. monocytogenes EGDe, and 53 probes present only in L. monocytogenes CLIP80459.

In a further preferred embodiment the hybridization support comprises the 409 probes listed in Table 7, or fragments or variants thereof.

In a further preferred embodiment the hybridization support comprises the 192 probes listed in Table 16, or fragments or variants thereof.

The invention also provides a method of serotyping a L. monocytogenes. The method comprises providing a sample comprising an L. monocytogenes of unknown serotype; providing an amplification primer pair for amplification of a 1/2a-1/2c-specific nucleic acid sequence; providing an amplification primer pair for amplification of a 1/2b-4b-specific nucleic acid sequence; providing an amplification primer pair for amplification of a 1/2c nucleic acid sequence; providing an amplification primer pair for amplification of a 4b nucleic acid sequence; performing an amplification reaction on the sample; and detecting the presence of any amplification products formed during the amplification reaction to thereby serotype the L. monocytogenes.

In some embodiments the 1/2a-1/2c-specific nucleic acid sequence is selected from Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 and Imo1974. In a preferred embodiment the 1/2a-1/2c-specific nucleic acid sequence is Imo737.

In some embodiments the 1/2b-4b-specific nucleic acid sequence is selected from ORF 2819, ORF 3840, ORF 2568, ORF 29 and ORF 1761. In a preferred embodiment the 1/2b-4b-specific nucleic acid sequence is ORF 2819.

In some embodiments the 1/2c nucleic acid sequence is selected from Imo151, Imo466, Imo467, Imo469, Imo470, Imo471, Imo1118 and Imo1119. In a preferred embodiment the 1/2c nucleic acid sequence is Imo1119.

In some embodiments the 4b nucleic acid sequence is selected from ORF 799, ORF 2372 and ORF 2110. In a preferred embodiment the 4b nucleic acid sequence is ORF 2110.

In a further preferred embodiment, the 1/2a-1/2c-specific nucleic acid sequence is Imo737, the 1/2b-4b-specific nucleic acid sequence is ORF 2819, the 1/2c nucleic acid sequence is Imo1119, and the 4b nucleic acid sequence is ORF 2110.

The invention also provides a kit for use in serotyping a L. monocytogenes. The kit comprises an amplification primer pair for amplification of a 1/2a-1/2c-specific nucleic acid sequence; an amplification primer pair for amplification of a 1/2b-4b-specific nucleic acid sequence; an amplification primer pair for amplification of a 1/2c nucleic acid sequence; and an amplification primer pair for amplification of a 4b nucleic acid sequence.

In some embodiments the 1/2a-1/2c-specific nucleic acid sequence is selected from Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973and Imo1974. In a preferred embodiment the 1/2a-1/2c-specific nucleic acid sequence is Imo737.

In some embodiments the 1/2b-4b-specific nucleic acid sequence is selected from ORF 2819, ORF 3840, ORF 2568, ORF 29 and ORF 1761. In a preferred embodiment the 1/2b-4b-specific nucleic acid sequence is ORF 2819.

In some embodiments the 1/2c nucleic acid sequence is selected from Imo151, Imo466, Imo467, Imo469, Imo470, Imo471, Imo1118and Imo1119. In a preferred embodiment the 1/2c nucleic acid sequence is Imo1119.

In some embodiments the 4b nucleic acid sequence is selected from ORF 799, ORF 2372 and ORF 2110. In a preferred embodiment the 4b nucleic acid sequence is ORF 2110.

In a particularly preferred embodiment, the 1/2a-1/2c-specific nucleic acid sequence is Imo737, the 1/2b-4b-specific nucleic acid sequence is ORF 2819, the 1/2c nucleic acid sequence is Imo1119, and the 4b nucleic acid sequence is ORF 2110.

The invention also provides a method for sub-lineage typing of a L. monocytogenes in a sample, wherein the method comprises detecting the absence or presence of a L. monocytogenes sub-lineage specific nucleic acid sequence in the sample. In some embodiments the L. monocytogenes sub-lineage specific nucleic acid sequence is a L. monocytogenes sub-lineage II.1 specific nucleic acid sequence. In a preferred embodiment the L. monocytogenes sub-lineage II.1 specific nucleic acid sequence is ORF0799, ORF2372, or ORF2110; or a fragment or variant of ORF0799, ORF2372, or ORF2110.

The invention also provides a hybridization support comprising a L. monocytogenes sub-lineage specific nucleic acid sequence. In some embodiments the L. monocytogenes sub-lineage specific nucleic acid sequence is a L. monocytogenes sub-lineage II.1 specific nucleic acid sequence. In a preferred embodiment the L. monocytogenes sub-lineage II.1 specific nucleic acid sequence is ORF0799, ORF2372, or ORF2110; or a fragment or variant of ORF0799, ORF2372, or ORF2110.

The invention also provides a kit for use in a method for identifying the lineage of a L. monocytogenes in a sample, wherein the kit comprises a L. monocytogenes sub-lineage specific nucleic acid sequence. In some embodiments the L. monocytogenes sub-lineage specific nucleic acid sequence is a L. monocytogenes sub-lineage II.1 specific nucleic acid sequence. In a preferred embodiment the L. monocytogenes sub-lineage II.1 specific nucleic acid sequence is ORF0799, ORF2372, or ORF2110; or a fragment or variant of ORF0799, ORF2372, or ORF2110.

The invention also provides a method of designing a hybridization support for typing Listeria. The method comprises determining at least one combination of Listeria species, strains, lineages, or sublineages to be distinguished; and selecting a plurality of probes from Table 17, based on the data presented in Table 17, for inclusion in the hybridization support.

### BRIEF DESCRIPTION OF THE DRAWINGS

This invention will be more fully described with reference to the drawings, in which:

FIG. 1 shows Listeria genetic diversity. Red and black denote presence and absence of genes, respectively. A) The dendrogram shows estimates of genomic relationships of the 113 strains constructed by hierarchical cluster analysis with the program J-Express. Phylogenetic lineages and subgroups are indicated. B) Enlargements represent the blocs of lineage specific genes whose numbers are indicated on the right hand side. I: lineage I (sv 1/2a; 1/2c, 3a, 3c); II: lineage II (sv 4b, 4d, 4e, 1/2b, 3b); III: lineage III (sv 4a, 4c); I.1: sv 1/2a, 3a; I.2: sv 1/2, 3C; II.1: sv 4b, 4d, 4e; II.2: sv 1/2b, 3b.

FIG. 2 shows an evolutionary scheme of the different lineages and serovars of L. monocytogenes.

FIG. 3 shows a phylogenetic tree of L. monocytogenes serovar 4b strains. The phylogenetic tree was constructed based on gene presence and absence using the program MVSP3.1. Numbers correspond to strains whose charcteristics are given in Table 2. Epi indicates epidemic strain isolated from humans or the incriminated food source.

FIG. 4 shows PCR products obtained from serotyping reference strains of L. monocytogenes and other Listeria genus species in a multiplex PCR assay. L. monocytogenes serotype 1/2a (Lane 1); L. monocytogenes serotype 1/2b (Lane 2); L. monocytogenes serotype 1/2c (Lane 3); L. monocytogenes serotype 3a (Lane 4); L. monocytogenes serotype 3b (Lane 5); L. monocytogenes serotype 3c (Lane 6); L. monocytogenes serotype 4a (Lane 7); L. monocytogenes serotype 4b (Lane 8); L. monocytogenes serotype 4c (Lane 9); L. monocytogenes serotype 4e (Lane 10); L. monocytogenes serotype 7 (Lane 11); L. innocua (Lane 12); L. welshimeri (Lane 13); L. ivanovii (Lane 14); L. seeligeri (Lane 15); L. monocytogenes serotype 4d (Lane 16). Lane M: SmartLadder SF molecular weight marker. The sizes in bp are indicated on the left.

FIG. 5 shows sequence alignment of junction regions.

### DETAILED DESCRIPTION OF THE INVENTION

One of the first criteria developed to subdivide species of L. monocytogenes was the varying antigenic properties of distinct subpopulations. A scheme developed by Seeliger and Höhne described 13 serovars (18). Based on somatic antigens L. monocytogenes isolates were divided mainly into serogroups 1/2 and 4, and based on flagellar antigen combinations, each of these serogroups was subdivided into serovars 1/2a, b, c, and 4b or some other less common serovars (18). Today, this scheme is still in use and numerous studies have identified correlations between certain phenotypic or genetic features and specific serovars (7, 8, 10, 19). The combined analysis of the genome sequences of L. monocytogenes sv 1/2a, L. innocua (13), and the partial genome sequence of L. monocytogenes sv 4b and macroarray hybridizations of 113 Listeria DNAs described herein, substantiated this classification at the genomic level.

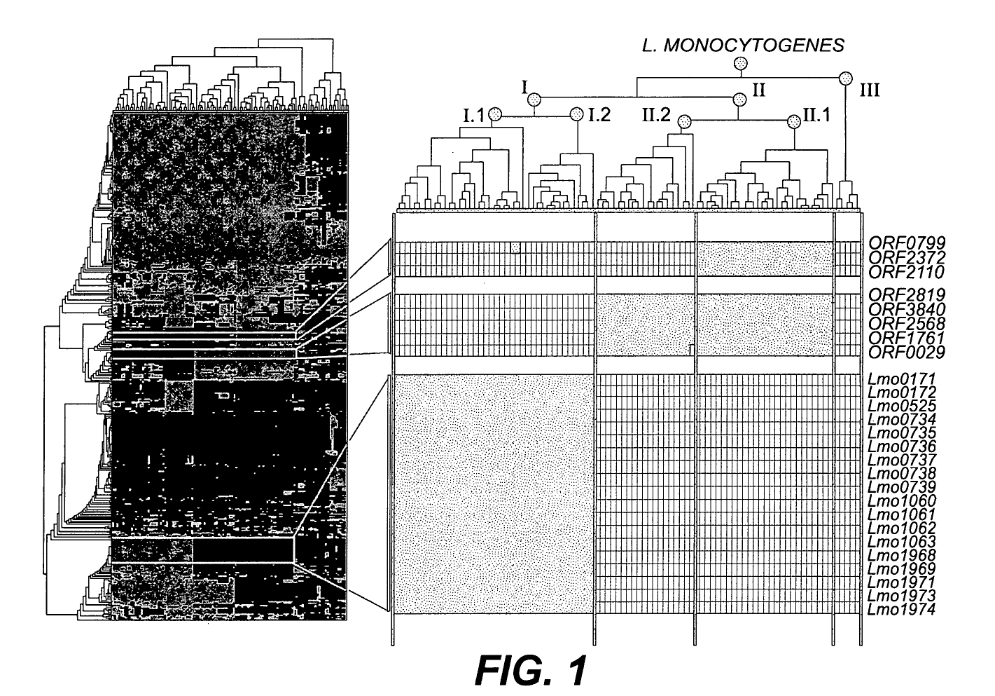

One of the most striking observations disclosed herein is the magnitude of divergence that exists within the species L. monocytogenes. The inventors have found that the genetic divergence between lineage I and lineage II of L. monocytogenes was nearly equally important (about 6%) as the inter-species differences between the sequenced L. monocytogenes EGDe sv 1/2a strain and L. innocua (10%). These results are in line with a previous report (20), which identified 39 specific gene fragments for the epidemic L. monocytogenes strain F.4565 as compared to L. monocytogenes EGDe using a subtractive hybridization method. This is of particular importance since strains of sv 4b mainly represent epidemic L. monocytogenes strains and are isolated from severe, invasive human cases more frequently than strains of other serovars, for example sv 1/2a. Apart from the important divergence between the two lineages of L. monocytogenes, the macroarray results identified a remarkable genomic conservation within the major lineages and subgroups (FIG. 1), but variations between the different subgroups. These results seem to mirror the evolution within the genus Listeria.

An important gene family in L. monocytogenes encodes surface proteins (13, 21). The macroarray hybridization data and the analysis of the partial L. monocytogenes sv 4b sequence described herein indicate that a group of surface proteins is highly specific for the species L. monocytogenes, which includes all previously known internalin genes (inlA, inlB, inlG, inlH, inlE, inlC, inlF). Furthermore, each sub-group of L. monocytogenes is characterized by a specific set of surface proteins. Finally, a third group of surface protein-coding genes is distributed quite heterogeneously among the different Listeria species. Interestingly, in the rarely isolated L. monocytogenes sv 4a strains, which are mostly of animal origin, 13 of the 25 L. monocytogenes specific surface proteins, including all internalins except inlAB, were missing. The lack of these proteins may be related to their lower disease potential in humans. The fact that different subgroups of L. monocytogenes strains contain different sets of surface proteins may also reflect their different potential to cause disease or to multiply in different niches. The elucidation of the functions of the different surface proteins and the putative strain specific characters they confer will be one of the challenging questions for the future and may give additional insights to understand the tropism of L. monocytogenes toward different cell types.

Proteins implicated in sugar transport and metabolism, in particular PTS systems, are another important gene family in Listeria (13). Analysis of the distribution of these genes again underlined the genetic divergence of the different sub-groups in L. monocytogenes, as each lineage was characterized by a specific set of PTS permeases. Most PTS genes present in sv 1/2a, 1/2c, 3a, and 3c strains and missing in L. innocua, were also missing in the sv 4b and 1/2b strains. The finding that the bvrABC locus, a β-glucoside specific PTS system, previously described as being implicated in virulence gene expression (14), was absent from all L. monocytogenes strains of lineage II (4b, 4d, 4e, 7, 1/2b, 3b) was surprising. As regulation of the PrfA regulon by β-glucosides also takes place in lineage II strains it can be assumed that another PTS system fulfills the functions of the bvrABC proteins. The finding that one of the PTS permeases identified in the sequence of the L. monocytogenes 4b strain was present in all strains where the bvrABC locus was lacking might be consistent with this hypothesis.

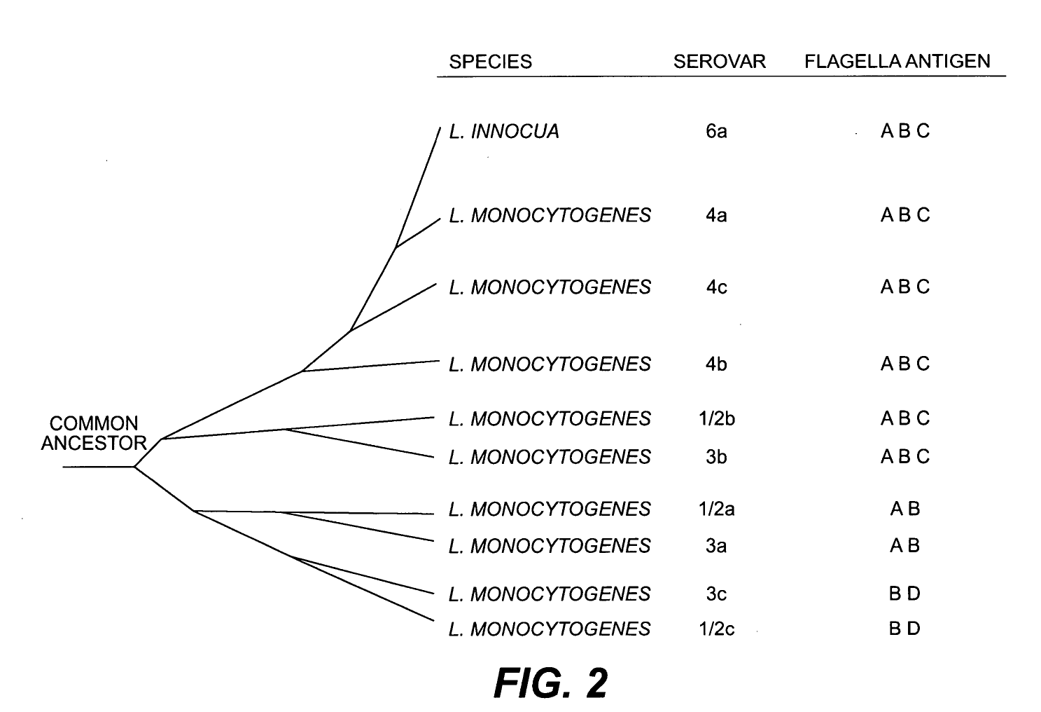

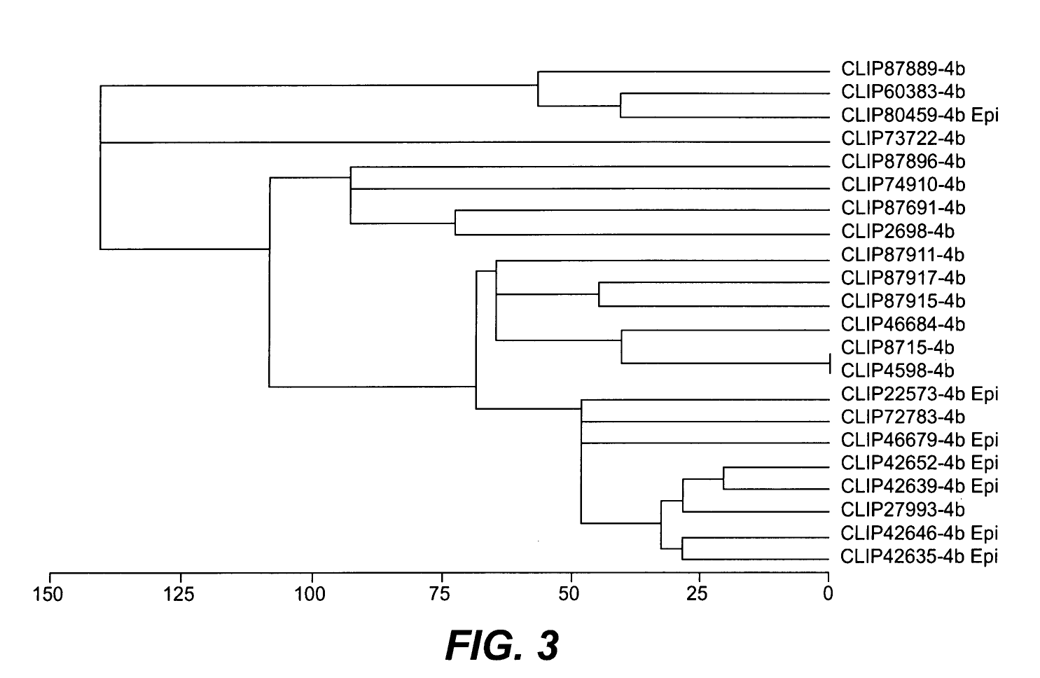

The inventors have identified 30 markers for the species L. monocytogenes (Table 9), as well as markers of each sub-population within the species L. monocytogenes (Table 10). One of the major questions is whether the pronounced differences in virulence among different subgroups of strains can be explained by different gene content. The results described herein revealed that known virulence genes (inlAB, prfA, plcA, hly, mpl, actA, plcB, uhpT and bsh) are present in all L. monocytogenes strains tested. However, analysis of the correlation between epidemiological data, origin of the strains, and the genomic profile clustered the L. monocytogenes sv 4b strains isolated from epidemics and the incriminated food source, in a group separate from the other environmental, food, or animal isolates (FIG. 2). Thus, disease related L. monocytogenes seem to be characterized by a particular combination of genes and the Listeria array combined with the knowledge of the marker genes identified in this study should prove as a powerful tool for identifying these strains. Examples of oligonucleotide pairs that can be used to amplify the marker genes are provided in Tables 14 and 15.

As shown in FIG. 1, the combined use of bioinformatics and macroarray results of 113 Listeria strains generated a large dataset, whose detailed analysis allows us to group strains according to shared genetic profiles. In addition to conclusions concerning genetics, epidemiology, and virulence of Listeria, these data also allow hypotheses regarding how the different Listeria species and phylogenetic lineages may have evolved. Several studies, analyzing the 16S and 23S rRNA (22, 23), PCR-based DNA fingerprinting techniques (24), or virulence locus and genome comparisons (5, 13, 25) indicated a phylogenetically close relationship between L. monocytogenes and L. innocua and suggested that L. innocua lost the virulence locus by deletion. Most interestingly, the inventors identified several other regions missing from L. monocytogenes sv 4a strains, which were also missing from L. innocua, like the inlGHE gene cluster. Sequence analysis of the different junction regions identified identical sequences among L. monocytogenes sv 4a and the L. innocua strains, suggesting single deletion events. The presence of these genes in the other Listeria species suggests that they were part of the genome of a common ancestor and that L. innocua evolved by successive gene loss from an ancestor of L. monocytogenes serogroup 4 strains. This hypothesis is also substantiated by the similar teichoic acid structure of L. monocytogenes serogroup 4 and L. innocua strains (17) and structural and functional similarity of the cell wall anchor of the autolysin Ami of L. monocytogenes serogroup 4 and L. innocua, but divergence between Ami of L. monocytogenes sv 1/2 and sv 4 (26). Further evidence for this close relationship also comes from the antigen structure of L. monocytogenes serogroup 4 and L. innocua which exhibit the same flagellar antigen structure, different from that of sv 1/2a and 1/2c strains (18). Based on this analysis and the literature, the inventors suggest an alternative model of the evolution within the L. monocytogenes-L. innocua branch. The separation into phylogenetic lineages is based on a divergence of sv 1/2c and sv 1/2b strains from a common ancestor (FIG. 2). Later in the evolution the sv 1/2b branch gained genes like for instance gtcA, which conferred serogroup-specific expression of TA-associated serotype specific antigens (27) and evolved into serogroup 4 and later on into the species L. innocua mainly by successive gene loss.

The DNA/DNA hybridization of a specific Listeria array containing genes of three different Listeria isolates showed that L. monocytogenes strains differ substantially in gene content. These differences are most pronounced in surface proteins and sugar metabolism, which are most likely to confer traits that provide selective advantages in the environment and the infected host. These results further provide an explanation why previous studies have found an association between various characteristics of L. monocytogenes and serovars. Thus, the inventors have demonstrated that this association is due to an evolutionary differentiation. To date, the microbiological surveillance of listeriosis, a disease that causes the death of at least 400-500 persons per year in Europe and North America, is based on subtyping by serotyping and pulsed-field gel electrophoresis (28). The precise characterization of L. monocytogenes is essential to following long-term trends in sporadic cases, as well as to detecting clusters of cases and epidemics and to identifying their common source. As such, the selective markers for the different subpopulations are an essential contribution for the construction of rapid, accurate identification and subtyping tools, and should be a powerful tool applicable in health institutions and the food industry. Finally, the identification of genes consistently absent or present in epidemic-associated L. monocytogenes strains now opens the way for mutational and functional analysis of these genes in order to decipher the molecular basis for the increased pathogenic potential of certain L. monocytogenes strains.

The evaluation of the PCR assay on 180 Listeria isolates listed in Table 1 permitted the inventors to separate without ambiguities the four major serovars 1/2a, 1/2b, 1/2c and 4b specifically within the pathogenic L. monocytogenes thus confirming the suitability of using this method to replace conventional serotyping. With a high specificity to the species L. monocytogenes, the PCR assay provides an additional species confirmation for L. monocytogenes isolates without the necessity of biochemical identification.

The PCR method described herein is preferred to conventional serotyping for its adaptability for a large scale routine analysis since at least 96 samples can be processed simultaneously, and is useful for a rapid subgroup characterization of the L. monocytogenes in the investigation of listeriosis outbreaks. Compared to standard agglutination assays, the PCR assays are faster to perform, with results obtainable within 4 to 6 hours. Furthermore, the materials used for this method are easily obtainable, while specific antisera required for traditional methods are costly and for this reason can not be widely utilized in many laboratories. The PCR assay is highly reproducible and the results are easy to interpret, and need no highly specialized expertise that may involve discrepancy problems arising from interpretation variability due to visual determination of agglutinations.

Importantly, because data collected by the National Reference Centre showed that the percentage of serovars 1/2a, 1/2b, 1/2c and 4b represented over 98% of 4000 isolates collected from foods and human patients over the last three years, the PCR method described herein can identify substantially all isolates of L. monocytogenes collected from foods and human patients, even when it is not possible to provide separation between serovars 3a and 1/2a or 3c and 1/2c or 3b, 7 and 1/2b neither between 4d, 4e and 4b.

A nucleic acid is a nucleotide polymer. As used herein “nucleic acid” refers interchangeably to nucleotide polymers composed of deoxyribonucleotides, ribonucleotides, or a mixture of both. The nucleic acid may comprise modified nucleotides. Modified nucleotides include nucleotides that have been labeled with a detectable moiety, such as a fluorescent or radioactive group. The nucleic acids of the invention may be isolated form a natural source or may be synthesized chemically using well known techniques in the art.

In preferred embodiments, the nucleic acid is isolated or purified. “Isolated” or “purified” refers in relation to a nucleic acid to a nucleotide polymer in the form of a separate fragment or as a component of a larger nucleic acid construct, which has been derived from nucleic acid isolated from its natural environment at least once.

The terms “isolated” or “purified” refer to a molecule that is substantially free from its natural environment. For instance, to a nucleotide polymer in the form of a separate fragment or as a component of a larger nucleic acid construct, which has been derived from nucleic acid isolated from its natural environment at least once. An “isolated” protein is substantially free of cellular material or other contaminating proteins from the cell or tissue source from which it is derived. The phrase “substantially free of cellular material” refers to preparations where the isolated protein is at least 70% to 80% (w/w) pure, more preferably at least 80%-89% (w/w) pure, even more preferably 90-95% pure; and most preferably at least 96%, 97%, 98%, 99% or 100% (w/w) pure.

A “nucleotide sequence” is a portion of nucleic acid comprising a defined nucleotide sequence. As used herein, a reference to a particular nucleotide sequence encompasses the complement of the references sequence. A particular nucleotide sequence may comprise deoxyribonucleotides, ribonucleotides, or a mixture of both, as well as modified nucleotides. Based on this definition, it is clear to one of skill in the art that “SEQ ID NO:1” refers to SEQ ID NO:1, the complement of SEQ ID NO:1, SEQ ID NO:1 composed of deoxyribonucleotides, ribonucleotides, or a mixture of both.

A “fragment” of a nucleotide sequence is a portion less than the whole. In preferred embodiments a fragment comprises at least about 8-12 nucleotides, at least about 15-25 nucleotides, at least about 20-50 nucleotides, at least about 25-100 nucleotides, at least about 100-1000 (1 kb) nucleotides or at least about 1-5 kb.

A “variant” of a nucleotide sequence is a nucleotide sequence that exhibits, with respect to the reference nucleotide sequence, certain modifications, such as in particular a deletion, a truncation, an extension, a chimeric fusion and/or a substitution, in particular a point substitution. A variant nucleotide sequence will generally exhibit at least 75%, preferably 80%, 85%, 90%, 95% or 98%, identity, after optimal alignment, with the reference nucleic acid sequence. A variant is preferably a sequence whose complementary sequences are capable of hybridizing specifically with the reference sequence. Preferably, the specific or high stringency hybridization conditions will be such that they ensure at least 75%, preferably 80%, 85%, 90%, 95% or 98%, identity, after optimal alignment, between one of the two sequences and the sequence complementary thereto.

The term “degenerate variant” of a reference nucleic acid sequence refers to all nucleic acid sequences that can be directly translated, using the standard genetic code, to provide an amino acid sequence identical to that translated from the reference nucleic acid sequence.

Hybridization under high stringency conditions means that the conditions of temperature and of ionic strength are chosen such that they allow the hybridization between two complementary DNA fragments to be specifically maintained. By way of illustration only, one of many possible high stringency conditions in the hybridization step for the purposes of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaC1 +0.015 M sodium citrate), 50% of formamide, 7% of sodium dodecyl sulfate (SDS), 10×Denhardt's, 5% of dextran sulfate and 1 % of saImon sperm DNA; (2) actual hybridization for 20 hours at a temperature which depends on the length of the probe (i.e.: 42° C. for a probe >100 nucleotides in length), followed by 2 washes for 20 minutes at 20° C. in 2×SSC +2% SDS, 1 wash for 20 minutes at 20° C. in 0.1×SSC +0.1% SDS. The final wash is carried out in 0.1×SSC +0.1 % SDS for 30 minutes at 60° C. for a probe >100 nucleotides in length. The high stringency hybridization conditions described above for a polynucleotide of defined length can be adjusted by those skilled in the art for longer or shorter oligonucleotides, according to the teaching of Sambrook et al. (1989, Molecular Cloning: a laboratory manual, 2nd Ed. Cold Spring Harbor).

The terms “polypeptide” and “protein”, used interchangebly herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The invention also provides fragments of the subject polypeptides. In some embodiments, fragments exhibit one or more activities associated with a corresponding naturally occurring polypeptide. Fragments find utility in generating antibodies to the full-length polypeptide; and in methods of screening for candidate agents that bind to and/or modulate polypeptide activity. The term “polypeptide composition” as used herein refers to the full-length bacterial protein as well as portions or fragments thereof. Also included in this term are variations of the naturally occurring bacterial protein, where such variations are homologous or substantially similar to the naturally occurring protein, as described in greater detail below, as well as corresponding homologs from other organisms. In the following description of the subject invention, the terms “polypeptide” are used to refer not only to the L. monocytogenes serovar 4b forms of these novel polypeptides, but also to homologs thereof expressed in other organisms.

In some embodiments, a polypeptide of the invention comprises at least about 10, at least about 20, at least about 25, at least about 30, at least about 40, at least about 50, at least about 60, at least about 70, at least about 75, at least about 80, or at least about 90 contiguous amino acids of one of the sequences shown in SEQ ID NOS: 164-326, up to the entire amino acid sequence of a sequence shown in SEQ ID NOS: 164-326.

Fragments of the subject polypeptides, as well as polypeptides comprising such fragments, are also provided. Fragments of polypeptide of interest will typically be at least about 10 amino acids (aa) in length, usually at least about 50 aa in length, and may be as long as 80 aa in length or longer, where the fragment will have a stretch of amino acids that is identical to the subject protein of at least about 10 aa, and usually at least about 15 aa, and in many embodiments at least about 50 aa in length.

Specific fragments of interest include those with enzymatic activity, fragments that bind to other proteins, fragments that bind to DNA, fragments with immunogenic or antigenic properties, and the like.

The invention provides polypeptides comprising such fragments, including, e.g., fusion polypeptides comprising a subject polypeptide fragment fused in frame (directly or indirectly) to a heterologous protein. Suitable heterologous proteins include, but are not limited to, a protein that serves as a detectable marker (e.g., a fluorescent protein, β-galactosidase, luciferase); an immunologically detectable protein (e.g., an epitope tag); and a structural protein.

Polypeptide fragments, such as those described above, are useful in screening assays, to identify agents that modulate an activity of a subject polypeptide.

The term “antibody” refers to one or more polyclonal antibodies, monoclonal antibodies, antibody compositions, antibodies having mono- or poly-specificity, humanized antibodies, single-chain antibodies, chimeric antibodies, CDR-grafted antibodies, antibody fragments such as Fab, F(ab′)2, Fv, and other antibody fragments which retain the antigen binding function of the parent antibody.

The term “monoclonal antibody” refers to one or more antibodies from a substantially homogeneous antibody population. The term encompasses whole immunoglobulins, fragments such as Fab, F(ab′)2, Fv, and other fragments that retain the antigen binding function of the antibody. Monoclonal antibodies are highly specific, being directed against a single antigenic epitope.

An antibody is said to “specifically bind” or “specifically react” with a protein or peptide molecule if such binding is not competitively inhibited by the presence of non-related molecules (e.g., bovine serum albumin).

Antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains (Clothia et al. (1985) J. Mol. Biol., 186: 651-663); Novotny and Haber (1985) Proc. Natl. Acad. Sci. USA, 82: 4592-4596).

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgGl, IgG2, IgG3, IgG4, IgA, and IgA2. The heavy-chain constant domains that correspond to the different classes of immunoglobulins are called a, d, e, g, and m, respectively. The subunit structures and three-dimensional configurations of different classes of immunoglobulins are well known in the art.

Antibodies may be raised against any portion of a protein which provides an antigenic epitope. The presently disclosed antibodies preferably bind to an epitope on a polypeptide sequence selected from SEQ ID NOS: 164-326; and more preferably from a fragment or variant of polypeptide sequence selected from SEQ ID NOS: 164-326.

A “hybridization support” is any substrate that a nucleic acid, polypeptide, or antibody of the invention may be attached to for use in an assay comprising a hybridization step. A hybridization support can be porous or solid, planar or non-planar, unitary or distributed. The bond between the nucleic acid or polypeptide and the substrate can be covalent or non-covalent.

Hybridization supports include, but are not limited to, a membrane, such as nitrocellulose, nylon, positively-charged derivatized nylon; a solid substrate such as glass, amorphous silicon, crystalline silicon, plastics (including e.g., polymethylacrylic, polyethylene, polypropylene, polyacrylate, polymethylmethacrylate, polyvinylchloride, polytetrafluoroethylene, polystyrene, polycarbonate, polyacetal, polysulfone, cellulose acetate, or mixtures thereof).

Nucleic acids, polypeptides, and antibodies of the invention can be attached covalently a surface of the hybridization support or applied to a derivatized surface in a chaotropic agent that facilitates denaturation and adherence, e.g., by noncovalent interactions, or some combination thereof.

In a preferred embodiment, a hybridization support of the invention comprises multiple nucleic acids or polypeptides of the invention attached to a single support, such as a single piece of nitrocellulose membrane or a single glass slide, in an array format, each nucleic acid having a unique physical location on the hybridization support. Such arrays differ mainly by their size, the material of the support and, optionally, the number of nucleic acids which are attached thereto.

The present invention provides vectors comprising a nucleic acid of the invention. In embodiments, the vectors are recombinant expression vectors to amplify a nucleic acid of the invention and/or to express a polypeptide product encoded by a nucleic acid of the invention. Recombinant expression vectors are replicable nucleic acid constructs that have a nucleic acid of the invention, the ability to replicate in a host, usually conferred by an origin of replication, and a selection gene to facilitate recognition of transformants.

A recombinant cell comprising a vector of the invention is a cell that has been transduced or transfected with the vector using recombinant DNA techniques. Suitable host cells include prokaryotes, yeast, or higher eukaryotic cells under the control of appropriate promoters. Prokaryotes include gram-negative or gram-positive organisms, for example E. coli or bacilli. Higher eukaryotic cells include established cell lines of mammalian origin.

A nucleic acid is detected in a sample when its presence is determined. For example, a nucleic acid may be identified by hybridizing a first nucleic acid having a first nucleotide sequence to the sample, and determining whether the first nucleotide sequence forms a hybrid with a complementary nucleotide sequence of a nucleic acid present in the sample. Because a reference to a nucleotide sequence encompasses a reference to its complement, a particular nucleotide sequence can be said to be detected through hybridization with itself.

An L. monocytogenes-specific nucleic acid is a nucleic acid comprising a nucleotide sequence present in L. monocytogenes but absent in other species of Listeria.

The “lineage” of a L. monocytogenes is a classification grouping based on the scheme set forth in Table 10. The lineages of L. monocytogenes include lineages I, II, and III.

An L. monocytogenes lineage-specific nucleic acid is a nucleic acid comprising a nucleotide sequence present in one lineage of L. monocytogenes but absent from the others (an example is ORF2819), or a nucleic acid comprising a nucleotide sequence absent in one lineage of L. monocytogenes but present in the others (an example is Imo94).

The “sub-lineage” of L. monocytogenes is a classification grouping based on the scheme set forth in Table 10. The sub-lineages of L. monocytogenes include sub-lineages I.1, I.2, II.1, II.2, III.1, and III.2.

An L. monocytogenes sub-lineage-specific nucleic acid is a nucleic acid comprising a nucleotide sequence present in one sub-lineage of a L. monocytogenes lineage, but absent from the other sub-lineage of the L. monocytogenes lineage. An example is ORF2110.

An L. monocytogenes sub-lineage-specific nucleic acid combination refers to a set of nucleic acids, the presence and/or absence of which, allows the classification of the strain of L. monocytogenes into a particular L. monocytogenes sub-lineage. For example, Imo737, Imo1119, ORF 2819, and 2110 are one such set.

“Primer” refers to a fragment of a nucleic acid sequence that is intended for use in a nucleic acid amplification reaction, such as PCR (polymerase chain reaction) technique (Rolfs et al., 1991, Berlin: Springer-Verlag). This technique requires the choice of pairs of oligonucleotide primers framing the fragment which must be amplified. Such a pair is referred to as an “amplification primer pair.” Reference may, for example, be made to the technique described in U.S. Pat. No. 4,683,202. The amplified fragments can be identified, for example after agarose or polyacrylamide gel electrophoresis or after a chromatographic technique such as gel filtration or ion exchange chromatography, and then sequenced. The amplified nucleotide fragments can be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques for amplifying a target nucleic acid can advantageously be used as an alternative to a PCR (PCR-like) using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, this involves amplification of the DNA with a polymerase; when the sample of origin is an RNA, a reverse transcription should be carried out beforehand. A very large number of methods currently exist for this amplification, such as, for example, the SDA (Strand Displacement Amplification) technique (Walker et al., 1992, Nucleic Acids Res. 20:1691), the TAS (Transcription-based Amplification System) technique described by Kwoh et al. (1989, Proc. Natl. Acad. Sci. USA, 86, 1173), the 3SR (Self-Sustained Sequence Replication) technique described by Guatelli et al. (1990, Proc. Natl. Acad. Sci. USA, 87:1874), the NASBA (Nucleic Acid Sequence Based Amplification) technique described by Kievitis et al. (1991, J. Virol. Methods, 35, 273), the TMA (Transcription Mediated Amplification) technique, the LCR (Ligase Chain Reaction) technique described by Landegren et al. (1988, Science 241,1077), the RCR (Repair Chain Reaction) technique described by Segev (1992, C. Kessler, Springer Verlag, Berlin, New-York, 197-205), the CPR (Cycling Probe Reaction) technique described by Duck et al. (1990, Biotechniques, 9, 142), and the Q-beta-replicase amplification technique described by Miele et al. (1983, J. Mol. Biol., 171,281). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA contained in the biological sample. The cDNA obtained will then be used as a target for the primers or the probes used in the method of amplification or of detection according to the invention.

The invention further provides a kit comprising a nucleic acid, or a fragment or derivative thereof, as described above. In embodiments the kit comprises two fragments of a single nucleic acid of the invention and the fragments comprise a primer pair suitable for use to amplify a third fragment of the nucleic acid using an amplification method, such as PCR. The nucleic acid(s) in the kit are present in a suitable storage medium, e.g., buffered solution, typically in a suitable container. In some embodiments the nucleic acids are in dried form, and may be provided lyophilized. In embodiments the kit comprises a hybridization support of the invention. The kit may further include a buffer; reagents for polymerase chain reaction (e.g., deoxynucleotide triphosphates (dATP, dTTP, dCTP, and dGTP), a thermostable DNA polymerase, a buffer suitable for polymerase chain reaction, a solution containing Mg2+ ions (e.g., MgCl₂), and other components well known to those skilled in the art for carrying out a polymerase chain reaction). The kit may further include instructions for use of the kit that may be provided in a variety of forms, e.g., as printed information, on a compact disc, or the like. The kit may further include reagents necessary for extraction of nucleic acids from a sample (e.g., biopsy sample, blood, and the like) from an individual or a food product. The kits are useful in diagnostic applications, as described in more detail below. The kits are useful, for example, to detect the presence of a listeria contamination, in particular a L. monocytogenes contamination, and more particularly a L. monocytogenes serovar 4b contamination. The kits are also useful to determine whether an isolate of listeria belongs to the species L. monocytogenes and, if so, to determine the lineage and sublineage of the listeria. A kit of the invention is typically provided in packaged form.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The following examples illustrate certain aspects of the invention. The examples in no way limit the invention.

### EXAMPLES

The following experimental techniques and reagents were used in Examples I-VIII.

Bacterial Strains: The Listeria strains used were selected from the culture collection of the National Reference Center for Listeria, Institut Pasteur/Paris and from the production environment of different food plants. The strains were selected to represent all sv of L. monocytogenes, as well as disease and non-disease related isolates. A total of 93 L. monocytogenes strains of 12 different sv isolated from humans (sporadic and epidemic cases), foods, animals, and the environment as well as 20 representative strains of the different species of the genus Listeria (eight L. innocua, six L. ivanovii, two L. weishimeri, two L. seeligeri, and two L. grayi strains) were studied (Table 2). This set included eight epidemic strains from five outbreaks, 15 isolates from sporadic cases, and 19 strains from food plants. Strains were routinely grown overnight at 37° C. without agitation in TPB broth (GIBCO).

Strain CLIP 80489 was deposited at the CNCM on Sep. 10, 2003, under Accession No.1-3083. The deposit receipt is attached hereto.

Sequencing, Assembly and Sequence Analysis: The complete nucleotide sequence of strain CLIP 80489 was determined as described by Glaser et. al. (2001). In a first phase 35 609 shot-gun sequences were assembled into 334 contigs by using the Phred-Phrap-Consed software package (Gordon D, Abajian C, Green P., 1998, Consed: a graphical tool for sequence finishing. Genome Res. Vol. 8, pp 195-202). These sequences were obtained by sequencing both ends of plasmid clones from a shot-gun library constructed in pdDNA2.1 (Invitrogen). Ordering of contigs was performed by using CAAT-box (Frangeul et al, Bioinformatics, in press) and by comparison with the genome sequence of L. monocytogenes strain EGDe and L. innocua strain CLIP-1 1262. Finishing (gap closure and re-sequencing of low quality regions was performed by PCR amplification and sequencing of PCR products (830 sequencing reaction were performed for this second phase). The final sequence is 2 904 719 pb long. For annotation of the sequence, we used the program CMT-box (Frangeul et al. Bioinformatics, in press).

Primer design, PCR amplification and array construction: Primers were designed modified version of Primer 3 (CAAT-Box; Frangeul et al., unpublished) to amplify a fragment of 300 to 600 bp specific for each gene (melting temperature of 55-65° C., Eurogentec). Amplification reactions were performed in a 100 μl reaction volume containing 10-20 ng chromosomal DNA. The concentration and size of each PCR product was verified on agarose gels. For array preparation nylon membranes (Qfilter, GENETIX) were soaked in TE solution (10 mM Tris-pH 7, 1 mM EDTA, pH7.6). Spot blots of PCR products and controls were printed using a Opix robot (GENETIX). Following spot deposition, membranes were fixed for 15 min in 0.5 M NaOH, 1.5 M NaCl, washed briefly in distilled water and stored wet at −20° C. until use.

Hybridization: Genomic DNA was extracted using Quiagen DNeasy kit and radiolabeled using a Random Priming DNA Labeling kit (ROCHE). Labeling was performed with 500 ng of genomic DNA and 50 μCi ³³P-labeled dCTP (Amersham). Labeled probes were purified using Sephadex G-50 (ROCHE) or Qiaquick minicolumns (Quiagen). High density arrays were wetted in 2×SSC and pre-hybridized for 1 h in 10 ml of 5×SSPE [1.8×SSPE is 1.8M NaCl, 13 mM NaH₂PO₄, 87 mM Na₂HPO₄ and 15 mM EDTA (pH 8)], SDS 4%, 1×Denhardt's [Denhardt's 50× is 1% Ficoll, 1% Polyvinylpyrrolidone and 1% BSA] and lmg of denatured SaImon sperm DNA. Hybridization was performed overnight at 60° C. Membranes were washed twice at room temperature and twice at 60° C. in 0.5% SSPE, 0.2% SDS. Arrays were then sealed in polypropylene bags and exposed to a phosphor screen (Molecular Dynamics) for 24 h.

Verification of the specificity and quality of the macroarray: 15% of all PCR products were randomly chosen and sequenced. All 64 sequences corresponded to the expected PCR products. The membrane was then hybridized with chromosomal DNA isolated from the three Listeria strains used to amplify the probes (L. monocytogenes EGDe, L. innocua CLIP11262, L. monocytogenes CLIP80459) to test the quality and the correct spotting.

Data Analysis: For scanning a 445SI phosphorlmager (Molecular Dynamics) was used. The ArrayVision software (Imaging Research) was used for quantification of the hybridization intensities and for normalization. For each spot, the hybridization intensity value was normalized by dividing by the average of all significant intensity values on each filter. For ratio calculation a reference array was used, which was built by combing the average normalized data from three replicate hybridizations with the genomic DNA of L. monocytogenes EGDe, L. innocua CLIP11262 and L. monocytogenes CLIP80459 to the corresponding spots on the array. In order to define the cut off ratio for the presence of a gene the hybridization results for L. monocytogenes EGDe genes hybridized with L. innocua chromosomal DNA were analyzed. The threshold for the presence of a gene was defined as >0.3. This corresponds to a DNA similarity higher than >80% as verified by sequence comparisons of these genes in both genomes. The data were then converted into a binary score (at >0.3 a gene was scored as present =1, at <0.3 a gene was scored as absent =0). The binary data were analyzed by hierarchical clustering using the program J-Express [Dysvik, 2001 #2046], by neighbor joining using the program MVSP 3.1(Kovach Computing Services) and by intensive expert based data mining using Excel spread sheets.

Amplification reactions were performed in a 96-well plate in a final volume of 100 μl containing 2 U of Taq DNA polymerase (ROCHE, Boehringer), 0.2 mM of dNTPs (Perkin Elmer) and 0.5 mM of each of the primer sets. PCR was performed with an initial denaturation step at 94° C. for 3 min, 35 cycles of 94° C. for 0.40 min ; 53° C. for 1.15 min, 72° C. for 1.15 min and one final cycle of 72° C. for 7 min in a thermocycler (Icycler biorad). 5 μl of the reaction mixture was mixed to 3 μl of gel loading buffer and separated on a 1% agarose gel in 1× TBE. The PCR product was visualized by ethidium bromide staining.

### Example I

### Construction of a Macroarray Comprising Genes from Epidemic L. monocytogenes, L. monocytogenes EGDe and L. innocua Strains

The 2906 kb long genome sequence of the epidemic L. monocytogenes sv 4b strain CLIP80459 (lineage II) was compared to the complete 2944 kb long genome sequence of L. monocytogenes EGDe (sv 1/2a, lineage I). 163 of the 2788 CLIP80459 genes (including 13 pseudogenes) were missing in EGDe. (The sequences of these genes have been assigned SEQ ID NOS: 1-163 and are presented in the attached Table 3. The amino acid sequences of the proteins encoded by the open reading frames (ORF) of these genes have been assigned SEQ ID NOS: 164-326 and are presented in the attached Table 4.) Thus, the genetic diversity between the two L. monocytogenes isolates is about 6%, quite close to that between L. monocytogenes EGDe and L. innocua (10.5%), which belong to two different species. The CLIP80459-specific genes include 14 surface proteins with an LPXTG motif, 14 ABC transport proteins, 2 PTS systems and 6 transcriptional regulators, suggesting that epidemic L. monocytogenes strains differ substantially in gene content from the L. monocytogenes EGDe strain (Table 5)

To extend the comparison from the three sequenced strains to a large collection of strains, high-density membranes were designed that were mainly focused on genes specific for each sequenced Listeria strain. This approach was chosen to increase the discriminatory power of the array. The membrane contained 409 probes, including 262 that were specific for L. monocytogenes EGDe relative to L. innocua CLIP11262 and all virulence genes, 94 that were specific for L. innocua CLIP11262 relative to L. monocytogenes EGDe, and 53 that were present only in L. monocytogenes CLIP80459, but absent from L. monocytogenes EGDe and L. innocua CLIP11262. The 53 genes that were present only in L. monocytogenes CLIP80459 are listed in Table 6, as are exemplary primer pairs that can be used to amplify fragments of the genes. All 409 genes contained on the membrane are listed in Table 7, together with exemplary primer pairs that can be used to amplify fragments of the genes. A description of the functional categories of the genes in listed in Table 8. This membrane was used to analyze 113 Listeria isolates.

The data obtained from the experiment are summarized in Table 17. Numbers indicate the percentage of the presence of the gene in the different groups or species of Listeria and the different L. monocytogenes sub-lineages. 8 strains of L. innocua, 6 strains of L. ivanovii, 2 strains of L. welshimeri, 2 strains of L. seeligeri, 2 strains of L. grayi and a total of 93 strains of L. monocytogenes, were evaluated in this study. L. monocytogenes strains were separated in this table into six sub-lineages. 27 strains (sv1/2a, 3a) 12 strains (sv 1/2c, 3c) 27 strains (sv 4b, 4d, 4e) 20 strains 3b, 7) 3 strains (sv 4a) and 2 strains (sv 4c) represented sub-lineage I.1, I.2, II.1, II.2, III.1, and III.2, respectively. The percentage of presence of each gene within the indicated groups is relative to the number of strains studied in each of these subdivisions.

### Example II

### Strain Diversity and Overall Gene Distribution

Based on the macroarray hybridization data predicting the presence or absence of the studied genes, bifurcating trees illustrating possible phylogenetic relationships between the different Listeria were constructed, using the neighbor joining method. Important gene conservation within each species of the genus Listeria and within distinct groups of L. monocytogenes strains was identified. The analysis grouped all strains without exception according to their species. Thus, the Listeria array allows accurate species identification, although the probes were defined from only two Listeria species. Each species was defined by a combination of genes specifically present or absent.

For the species L. monocytogenes 30 marker genes were identified, 18 that were present in all 93 L. monocytogenes strains tested (Table 9, group I), and absent in all other isolates of the remaining Listeria species, and 12 that were present in all L. monocytogenes, except some or all of the five serovar 4a and 4c isolates tested (Table 9, group II). As serovar 4a and 4c strains are very rare and do not cause typical human listeriosis (12), these strains will not be considered in the analysis of species-specific marker genes but discussed separately. These 30 markers comprised the well-known virulence genes, (plcA, plcB, acta), seven surface protein-coding genes (inlA, inlB, inlH, inlE, Imo0333, Imo0835, Imo2821), one soluble internalin (Imo0549), three transcriptional regulators and eleven proteins of unknown function. For the species L. innocua we identified four markers (lin0739, lin0803, lin2741, lin2918) that were consistently present in all L. innocua strains tested. However, 29 of the 94 L. innocua genes spotted on the membrane were detected only in L. innocua, suggesting them to be species specific.

As the macroarray did not contain probes specific for the other Listeria species, no specific markers for these species could be defined. However, orthologs of about a third of the EGDe genes and about a quarter of the CLIP80459 or L. innocua genes were identified in at least one of the L. ivanovii, L. seeliger or L. whelshimeri strains. L. grayi was found to be the most distantly related species. From the 409 probes, only 12 (six from EGDe, three from L. innocua and three from CLIP80459) hybridized with DNA from L. grayi. These probes correspond to genes present in all Listeria species, like Imo1136, coding for an LPXTG protein, and genes coding for proteins of unknown function.

### Example III

### Sub-Grouping Within the Species L. monocytogenes

The neighbor-joining method and hierarchical clustering (J-Express) were applied to identify specific gene clusters. Analysis of the 93 L. monocytogenes strains defined three lineages (I, II, and III) and distinguished two subdivisions within each lineage (FIG. 1). For each lineage and subgroup, specific markers were identified.

Nineteen genes were associated specifically with lineage I (Table 10, group A). Twelve of these genes clustered in two regions coding for proteins putatively involved in sugar metabolism (Imo0734-Imo0739 and Imo1968-Imo1974). Furthermore, a two-component regulatory system (Imo1060, Imo1061), an ABC transporter complex (Imo1062, Imo1063) and a gene coding for a surface protein containing an LPXTG motif (Imo0171) were lineage I-specific. Surprisingly, the bvr locus (bvrABC) (14) was present only in isolates of lineage I and in the two 4c strains. Eight genes allowed the sub-division of lineage I. They were present in lineage I.2 (sv 1/2c and 3c) but generally absent from lineage I.1 (sv 1/2a and 3a) (Table 10, group B).

Five of the 53 sv 4b-specific genes were markers for lineage II (Table 10, group C). Two code for transcriptional regulators, and three for surface proteins containing an LPXTG anchor. As sv 4b strains are mainly responsible for human listeriosis, it is of particular interest to identify markers for sv 4b strains or for the subgroup 4b, 4d, and 4e (lineage II.1). One such specific marker was ORF0799, coding for an unknown protein. ORF2372 (putative teichoic acid protein precursor C) and ORF2110 (putative secreted protein) were present only in sv 4b and two or four of the six L. ivanovii strains, respectively. (These three genes are shown in Table 10, group D.) Furthermore, 35 of the 53 sv 4b genes spotted were conserved in all 4b strains, and are thus implicated in the determination of characteristic features of sv 4b strains.

For lineage III strains (sv 4a and 4c), no specific genes were identified, because the macroarray did not contain representative sequences of this lineage. However, lineage III was characterized by the absence of over 37% (96 genes) of the EGDe genes spotted on the membrane. Thirteen genes, clustered in eight different chromosomal regions, were specifically absent from lineage III strains. They code for surface proteins (Imo1666, Imo0835), the arginine metabolic pathway (Imo0036 -Imo0041) and proteins of unknown function Imo96, Imo832, Imo1441, Imo1451, Imo2671). Strains of sv 4a (lineage III.1) are distinguished from those of sv 4c (lineage III.2) by the lack of additional 20 genes, seven of which code for cell surface proteins (inlC, inlEHG, Imo0333, Imo0549, Imo2821). These genes were also absent from all L. innocua strains tested.

Based on the data described above and reported in Table 9, Table 10, and Table 17 it is possible to design additional hybridization supports for use in typing of comprising sets of genes for use in typing L. monocytogenes isolates. An example of such a support, and results obtained using the support, is provided in Table 16.

### Example IV

### Distribution of Known Virulence Genes

The virulence gene cluster of L. monocytogenes comprises prfA, plcA, hly, mpl, actA, and plcB. As these genes are a prerequisite for virulence of L. monocytogenes, differences in virulence among different isolates could be due to the absence of one or more of these genes. However, the macroarray hybridization showed that all 93 L. monocytogenes isolates contained this virulence gene cluster. The above-mentioned genes have been reported to also be present in L. ivanovii and L. seeligeri. In the macroarray assay, hybridization signals were detected for the hly, mpl and prfA genes, whereas the plcA, actA and plcB genes were either absent or did not give a signal due to a high divergence of the corresponding gene orthologs. Indeed, the sequence similarity of plcA, plcB, and actA from different L. ivanovii and L. seeligeri strains, as compared to L. monocytogenes EGDe, does not exceed 60%.

Several other genes of L. monocytogenes have been implicated in adhesion and internalization. Among those, the best studied are inlA and inlB. These two genes were detected in all L. monocytogenes strains tested, confirming their species-specificity. The uhpT gene (15) and the bsh gene (16) were identified in all isolates of the three hemolytic Listeria species (L. monocytogenes, L. ivanovii and L. seeligen).

### Example V

### High Diversity of Surface Proteins Within the Species L. monocytogenes

Fifty-five genes coding for putative surface proteins belonging to the three sequenced Listeria genomes were spotted on the array. Among these, two groups of genes were identified. The first one comprises 25 genes specific for the species L. monocytogenes, including inlAB, the inlGHE cluster, inlF, and a number of surface proteins of unknown function (Table 11). Two (Imo0171, Imo2026) are lineage I specific and three (ORF2568, ORF1761, ORF0029) are lineage II specific. lnlG seems to be specifically absent from all lineage II and sv 4a strains. None of the sv 4b surface protein-coding genes was identified in L. monocytogenes 1/2c and 3a strains. For L. innocua, two specific surface protein-coding genes (lin0739, lin0803) were identified. The second group comprises surface protein-coding genes heterogeneously distributed among the different Listeria isolates and species (Table L. monocytogenes 3).

To corroborate the hybridization results, six of the surface proteins specific for L. monocytogenes (inlA, inlB, inlE, inlG, inlH, inlF) and two of the surface proteins found within several or all Listeria species (Imo0550, Imo1289), were amplified by PCR in one reference strain for each sv and for each species. The PCR amplifications confirmed the array results.

### Example VI

### Carbohydrate Metabolism and Phosphotransferase Systems (PTS)

The distribution of genes for twelve PTS permeases and 14 genes coding proteins predicted to be implicated in sugar metabolism and degradation was similar to that of surface protein coding genes; all genes were highly conserved in lineage I strains and most were lacking in the other Listeria species. Except for two PTS genes (Imo2733, Imo2782) and three carbohydrate metabolism genes (Imo2143, Imo2735, Imo2781), all other genes were missing in the L. monocytogenes sv 4a strains.

### Example VlI

### Cell Wall Proteins—Two sub-divisions within Teichoic Acid Biosynthesis Genes

Despite the fact that the majority of genes grouped the 93 L. monocytogenes strains according to previously defined lineages, which correlate mainly with the flagellar antigen combinations (serovars), 13 genes implicated in cell wall biosynthesis where identified that divide the L. monocytogenes strains into two groups according to their somatic antigen (serogroups 4 and serogroup 1/2, 3, 7). These genes code for teichoic acid biosynthesis proteins and were detected only within the strains of serogroup 1/2, 3 and sv 7, but were absent from strains of serogroup 4. This finding is in agreement with previous studies that have identified two distinct structural types of teichoic acid within L. monocytogenes, where the first type was found in strains of serogroup 1/2, 3 and 7 and the second in strains of serogroup 4 (17). This suggests that these genes may be implicated in the synthesis of this specific teichoic acid type. Nine of these genes located within a 19 kb region of the L. monocytogenes chromosome (Imo1076-Imo1077, Imo1080-Imo1084, Imo1088, Imo1091), were also shared with the L. seeligeri strains, which are of serogroup 1/2, suggesting that L. seeligeri has a teichoic acid type similar to that of L. monocytogenes serogroup1/2.

Similarly, one (lin1073) of two L. innocua genes implicated in teichoic acid biosynthesis was uniquely shared with the L. monocytogenes strains of serogroup 4 and with L. weslhimeri. This is consistent with the suggestion that the cell wall type of the serogroup 4 strains of L. monocytogenes is more closely related to that of serogroup 6 of L. innocua than to that of L. monocytogenes 1/2 strains (17).

### Example VIII

### Variable Genomic Regions and Analysis of Junction Sequences

Several L. monocytogenes EGDe gene clusters were missing only from L. monocytogenes sv 4a strains but were present in all other L. monocytogenes strains. Two regions were absent from L. monocytogenes sv 4a and L. innocua, but were present in L. ivanovii, L. seeligeri and L. welshimeri. These regions seemed good candidates as evolutionary markers. To further analyze them, the junction regions of the putative deletion sites in six isolates of L. innocua and L. monocytogenes sv 4a were sequenced.

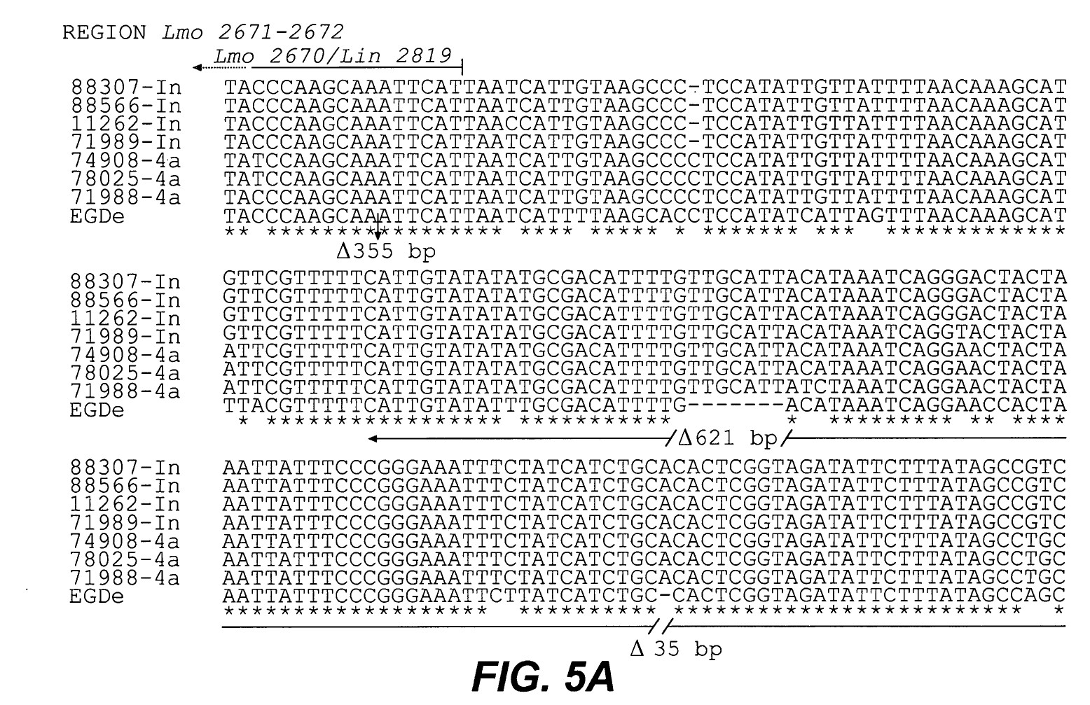

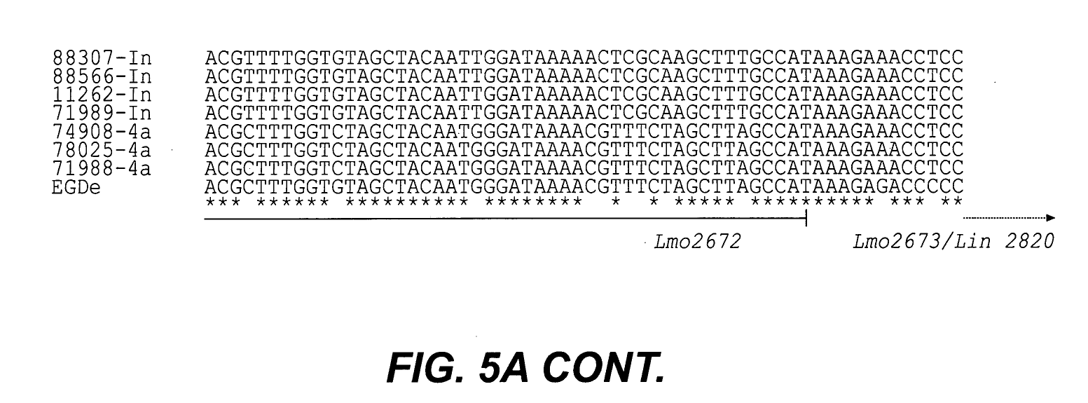

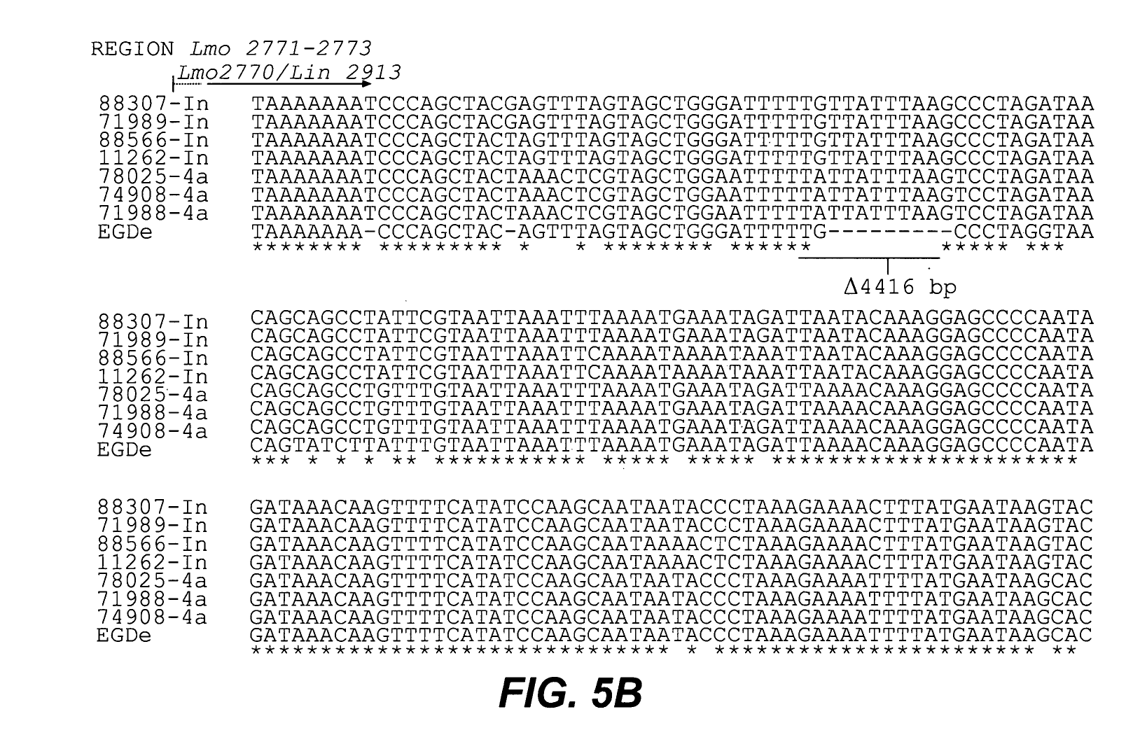

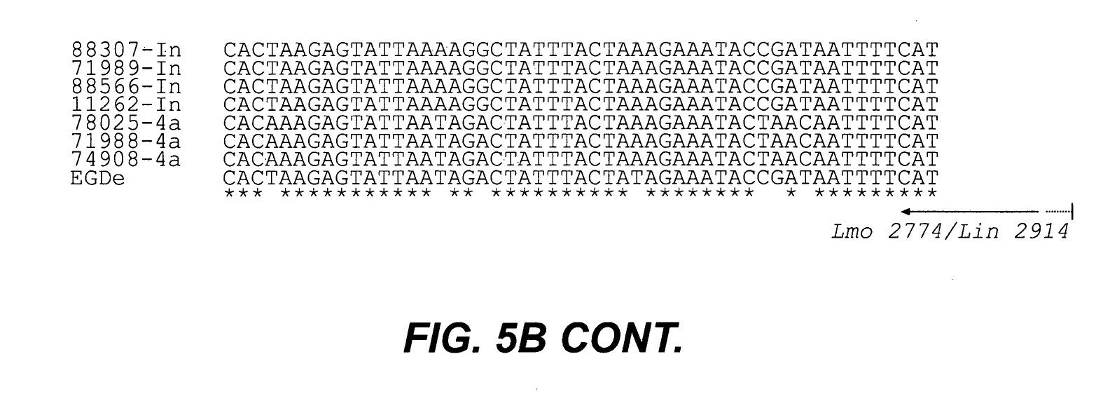

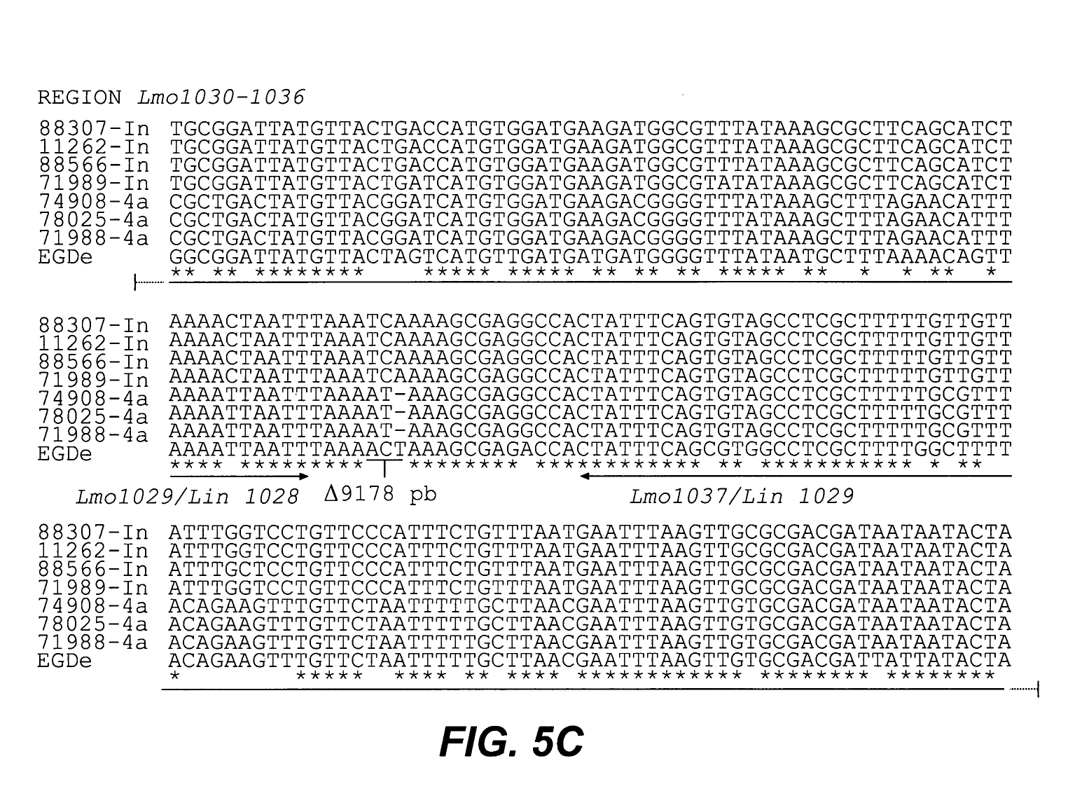

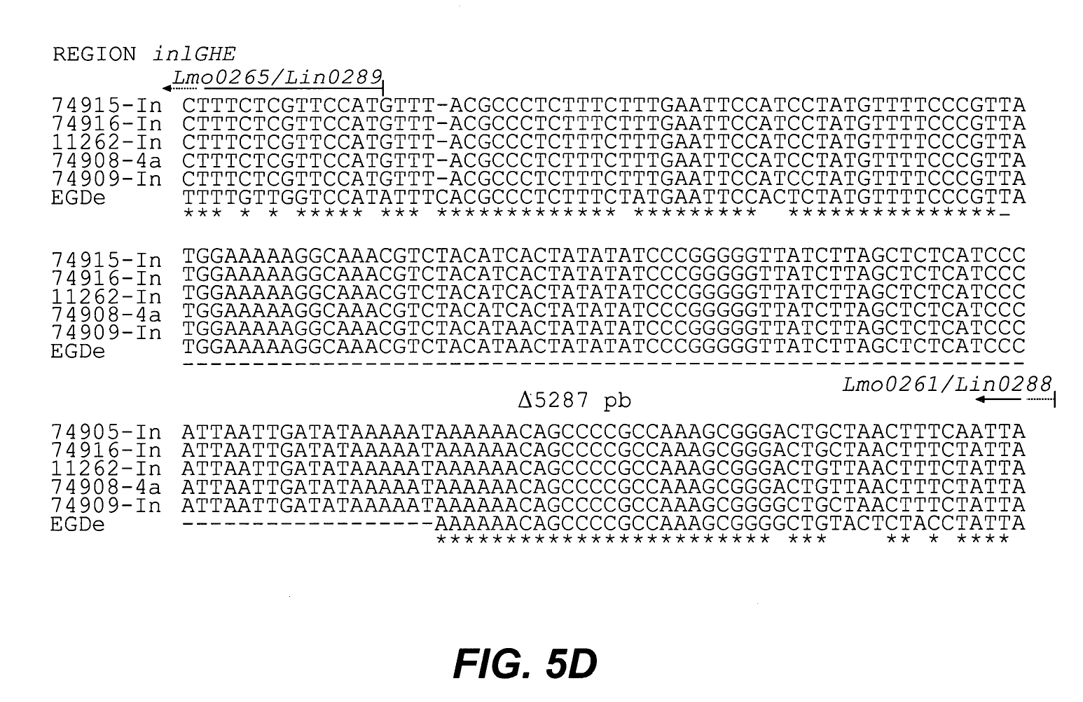

Analysis of region Imo2671- Imo2672 revealed the existence of three deletion events, two of which were located in the coding sequence of Imo2672, resulting in the deletion of two internal fragments of 621 bp and 35 bp. The third deletion was 355 bp long and located downstream of the 5′ end of the coding sequence of Imo2671. All three junction sequences were identical, containing either an insertion of TTGCATT, A or no insertion (FIG. 5A). An analysis of the junction sequence of region Imo2771-Imo2773 yielded the same result, with identical junction sites in the six strains sequenced 38 bp downstream of the 3′ end of Imo2770. The junction site had an insertion of the sequence TTATTTAAG replacing the genes Imo2771 to Imo2773 (FIG. 5B).

The third region investigated (Imo1030-Imo1036) was absent from L. innocua and L. monocytogenes 4a strains and present in L. ivanovii. The analysis of the junction region identified a minor sequence variation (insertion of TCA in L. innocua and of AT in L. monocytogenes sv 4a) at the deletion site (FIG. 5C).

Finally, the inlGHE cluster, which is missing in L. monocytogenes 4a and all other Listeria sp. was analyzed. Again, an identical sequence in the five strains sequenced was observed, suggesting that a single deletion event had occurred in a common ancestor of L. monocytogenes sv 4a and L. innocua (FIG. 5D).

### Example IX

### Multiplex PCR

The experiments described in the preceding examples identified nineteen genes from L. monocytogenes EGDe strain (Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 and Imo1974) and five genes for the L. monocytogenes CLIP80459 strain (ORF 2819, ORF 3840, ORF 2568, ORF 29 and ORF 1761) as a marker genes, based on their specific detection in L. monocytogenes strains of serovars 1/2a-1/2c and 1/2b-4b, respectively. Moreover, strains of serovars 1/2c and 4b were specifically distinguished by the presence of eight additional genes of L. monocytogenes EGDe (Imo151, Imo466, Imo467, Imo469, Imo470, Imo471, Imo1118 and Imo1119) and three genes of L. monocytogenes CLIP80459 (ORF 799, ORF 2372 and ORF 2110), respectively.

These marker genes can be used in a PCR assay that classifies L. monocytogenes into four distinct groups in correlation with the four major serovars (1/2a, 1/2b, 1/2c and 4b). Four L. monocytogenes serogroup marker genes were selected to develop the multiplex PCR assays. The tests were carried out in 96 well microplate format, allowing large scale application. A total of 160 strains of L. monocytogenes and 20 strains representing species L. innocua (10 strains of serovar 6a and 6b), L. ivanovii (8 strains of serovar 5), L. welshimeri (2 strains of serovar 6a and 4c) and L. seeligeri (2 strains of serovar 1/2b), were evaluated in this study (Table 1). Among L. monocytogenes strains, serovars 1/2a, 1/2b, 1/2c and 4b were each represented by 40 strains, half of which were isolated from foods and half from human sporadic cases of listeriosis and which were isolated over the period from 2000 to 2002 and collected by the National Reference Centre (NRC) at Institut Pasteur. Serovar reference strains were also used in this study. Strain identification and conventional serotyping were done according to standard methods (Bille et al. 1992; Seeliger and Höhne. 1979).

For PCR, three to five bacterial colonies were emulsified in 50 μl of a 0.25% sodium dodecyl sulfate-0.05 N NaOH solution and incubated at 99° C. for 15 min. Then, 100 μl of H₂O was added to the mixture and 2 μl of the diluted mixture was used in PCR. Multiplex PCR assays were performed by combining six primer sets in a single reaction. Two of these primer sets were designed to target marker gene Imo0737 of L. monocytogenes EGDe strain and ORF 2819 of L. monocytogenes CLIP 80459 strain, specific to L. monocytogenes serovars 1/2a-1/2c and 1/2b-4b respectively. To further distinguish L. monocytogenes serovar 1/2c from 1/2a and serovar 4b from 1/2b, two primer sets targeting the serovar 1/2c marker gene Lmo1119 of L. monocytogenes EGDe and the serovar 4b marker gene ORF2110 of L. monocytogenes CLIP 80459 strain, were included. Finally, prs gene was also targeted by a primer pair for an internal PCR amplification control, as it was highly conserved among all listeria species. Primer sequences and predicted lengths of amplified fragments are outlined in Table 12.

As a demonstration of the PCR approach, the four major serovars 1/2a, 1/2b, 1/2c and 4b of the pathogeneic L. monocytogenes species were separated based on the amplification of four of the serogroup marker genes already described.

The first set of primers was designed to amplify 691 bp fragment of gene Imo0737, a transcription regulator of the Lacl family of the L. monocytogenes EGDe strain, selected from the nineteen marker genes identified as specific for L. monocytogenes strains of serovars 1/2a, 1/2c, 3a and 3c. From the eight marker genes specific for L. monocytogenes strains of serovars 1/2c and 3c, primers were designed to amplify a 782 bp fragment of the DNA methyl transferase encoded by gene Imo1119 of L. monocytogenes EGDe. PCR primers for this assay were also designed to amplify a 471 bp fragment of the ORF 2819 of L. monocytogenes CLIP80459, a cell surface protein containing a LPXTG anchor motif identified with four other genes as specific markers for L. monocytogenes strains of serovars 1/2b, 3b, 7, 4b, 4d and 4e. The three marker genes characterizing L. monocytogenes strains of serovars 4b, 4d and 4e were tested in this study by the amplification of 600 bp fragment covering ORF 2110 of L. monocytogenes CLIP80459 strain, encoding a putative secreted protein.

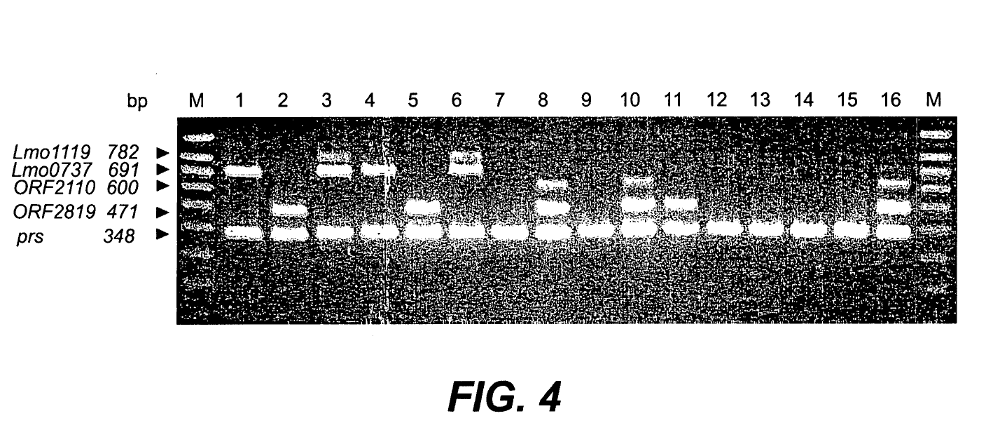

Thus, amplification of gene Imo1119 (782 pb band) and gene Imo0737 (691 pb band) indicates a 1/2c or 3c serovar, while the single amplification of gene Imo0737 (691 bp band) would indicate a 1/2a or 3a serovar (FIG. 4). In contrast, the amplification of ORF2819 (471 pb band) and ORF2110 (600 bp band) is associated with serovars 4b, 4d or 4e. The unique amplification of the ORF 2819 (471 bp band) indicates a serovars of 1/2b, 3b or 7 (FIG. 4). The suitability of the PCR method to detect serovars was first evaluated with the serovar reference strains of the L. monocytogenes species. As shown in (FIG. 4), the size of the amplicons obtained from the multiplex PCR was identical to those predicted from the design of the primers, and amplification profiles, as expected, separate serovars 1/2a-3a, 1/2c-3c, 1/2b-3b-7 and 4b-4d-4e. Thus, this PCR assay allows identification of species of L. monocytogenes as belonging to one of four distinct groups. Multiplex PCR profiles combine serovars 1/2a with 3a, 1/2c with 3c, 1/2b with 3b-7 and 4b with 4d-4e. However, the probability of such confusions will still be limited since the remaining six currently recognized L. monocytogenes serovars are infrequent in food and rarely reported to be implicated in human listeriosis (Tappero et al. 1995; Graves et al. 1999).

### Example X

### Specificity and Evaluation of a Representative Multiplex PCR Assay

To evaluate the specificity and the reproducibility of the multiplex PCR method, 160 strains of L. monocytogenes species, and 20 strains representing other listeria species were tested: L. innocua (10 strains), L. ivanovii (8 strains), L. welshimeri (2 strains) and L. seeliger (2 strains). The serovar classifications obtained from interpretation of the PCR results were then compared with those obtained using conventional serotyping methods. In all strains from the species L. innocua, L. ivanovii, L. seeligeri and L. welshimeri, the only amplified gene was the prs control (348 bp band) (FIG. 4). Results summarized in Table 13 show that the serovar classifications determined from the PCR results were in complete concordance with the classifications obtained using the conventional serotyping methods for all strains of the species L. monocytogenes assayed, confirming the reproducibility and accuracy of the PCR method.

### REFERENCES

(1) Perna, N. T. & al., e. (2001) Nature 409, 529-533.

(2) Alm, R. A., Ling, L. S., Moir, D. T., King, B. L., Brown, E. D., Doig, P. C., Smith, D. R. & Noonan, B. (1999) Nature 397, 176-180.

(3) Joyce, E. A., Chan, K., Salama, N. R. & Falkow, S. (2002) Nat Rev Genet 6, 462-473.

(4) Fitzgerald, J. R. & Musser, J. M. (2001) Trends Microbiol 11, 547-553.

(5) Vazquez-Boland, J. A., Kuhn, M., Berche, P., Chakraborty, T., Dominguez-Bernal, G., Goebel, W., Gonzalez-Zorn, B., Wehland, J. & Kreft, J. (2001) Clin Microbiol Rev 14, 1-57.

(6) Jacquet, C., Gouin, E., Jeannel, D., Cossart, P. & Rocourt, J. (2002) Appl Environ Microbiol. 68, 616-622.

(7) Piffaretti, J. C., Kressebuch, H., Aeschbacher, M., Bille, J., Bannerman, E., Musser, J. M., Selander, R. K. & Rocourt, J. (1989) Proc. Natl. Acad. Sci. USA 86, 3818-3822.

(8) Bibb, W. F., Schwartz, B., Gellin, B. G., Plikaytis, B. D. & Weaver, R. E. (1989) Int J Food Microbiol. 8, 233-239.

(9) Brosch, R., Chen, J. & Luchansky, J. B. (1994) Appl Environ Microbiol. 60, 2584-2592.

(10) Graves, L., Swaminathan, B., Reeves, M. & et al. (1994) J. Clin. Microbiol. 32, 2936-2943.

(11) Jeffers, G. T., Bruce, J. L., McDonough, P. L., Scarlett, J., Boor, K. J. & Wiedmann, M. (2001) Microbiology 147, 1095-1104.

(12) Wiedmann, M., Bruce, J. L., Keating, C., Johnson, A. E., McDonough, P. L. & Batt, C. A. (1997) Infect Immun. 65, 2707-2716.

(13) Glaser, P., Frangeul, L., Buchrieser, C., Rusniok, C., Amend, A., Baquero, F., Berche, P., Bloecker, H., Brandt, P., Chakraborty, T., Charbit, A., Chetouani, F., Couve, E., de Daruvar, A., Dehoux, P., Domann, E., Dominguez-Bernal, G., Duchaud, E., Durant, L., Dussurget, O., Entian, K. D., Fsihi, H., Portillo, F. G., Garrido, P., Gautier, L., Goebel, W., Gomez-Lopez, N., Hain, T., Hauf, J., Jackson, D., Jones, L. M., Kaerst, U., Kreft, J., Kuhn, M., Kunst, F., Kurapkat, G., Madueno, E., Maitournam, A., Vicente, J. M., Ng, E., Nedjari, H., Nordsiek, G., Novella, S., de Pablos, B., Perez-Diaz, J. C., Purcell, R., Remmel, B., Rose, M., Schlueter, T., Simoes, N., Tierrez, A., Vazquez-Boland, J. A., Voss, H., Wehland, J. & Cossart, P. (2001) Science 294, 849-852.

(14) Brehm, K., Ripio, M. T., Kreft, J. & Vazquez-Boland, J. A. (1999) J Bacteriol 181, 5024-32.

(15) Chico-Calero, I., Suarez, M., Gonzalez-Zorn, B., Scortti, M., Slaghuis, J., Goebel, W. & Vazquez-Boland, J. A. (2002) Proc Natl Acad Sci USA 99, 431-436.

(16) Dussurget, O., et al., (2002) Molecular Microbiology 45, in press.

(17) Fiedler, F. (1988) Infection 16, 92-97.

(18) Seeliger, H. P. R. & Hohne, K. (1979) in Methods in Microbiology, eds. Bergan, T. & Norris, J. R. (Academic press, London), Vol. 13.

(19) Brosch, R., Buchrieser, C. & Rocourt, J. (1991) Res. Microbiol. 142, 667-675.

(20) Herd, M. & Kocks, C. (2001) Infect immun 69.

(21) Cabanes, D., et al., (2002) Trends Microbiol 5, 238-245.

(22) Collins, M. D., Wallbanks, S., Lane, D. J., Shah, J., Nietupski, R., Smida, J., Dorsch, M. & Stackebrandt, E. (1991) Int J Syst Bacteriol 41, 240-246.

(23) Sallen, B., et al., (1996) Int J Syst Bacteriol 46, 669-674.

(24) Vaneechoutte, M., et al., (1998) Int J Syst Bacteriol 48,127-139.

(25) Chakraborty, T., Hain, T. & Domann, E. (2000) Int J Med Microbiol 2, 167-174.

(26) Milohanic, E., Jonquières, R., Glaser, P., Berche, P., Cossart, P. & Gaillard, J. L. Mol Mibrobiol in press.

(27) Promadej, N., Fiedler, F., Cossart, P., Dramsi, S. & Kathariou, S. (1999) J Bacteriol 181, 418-425.

(28) Graves, L. M. & Swaminathan, B. (2001) Int J Food Microbiol 65, 55-62.

Bille, J. B., et al., 1992. API Listeria, a new and promising one-day system to identify Listeria isolates. Appi Environ Microbiol. 58:1857-60.

Brosch R., et al., 1996. Genomic fingerprinting of 80 strains from the WHO multicenter international typing study of listeria monocytogenes via pulsed-field gel electrophoresis (PFGE). Int J Food Microbiol 32: 343-55.

Bruce J. L., et al., 1995. Sets of EcoRI fragments containing ribosomal RNA sequences are conserved among different strains of Listeria monocytogenes. Proc Natl Acad Sci U S A. 92: 5229-33.

Farber J. M. and P. 1. Peterkin. 1991. Listeria monocytogenes: a food-borne pathogene. Microbiol. Rev. 55: 476-511.

Glaser P., et al., 2001. Comparative genomics of Listeria species. Science. 294:849-52.

Graves L. M., et al., 1999. in Listeria, Listeriosis and food safety, p 251-297 in 2nd Ed. Ryser E. T. and E. H. Marth (ed.). Marcel Dekker, Inc., New York.

Schonberg A, et al., 1996. Serotyping of 80 strains from the WHO multicentre international typing study of Listeria monocytogenes. Int J Food Microbiol. 32: 279-87.

Seeliger H. P. R., and K. Höhne. 1979. Serotyping of L. monocytogenes and related species. Methods Microbiol. 13: 31-49.

Tappero J W, et al., Reduction in the incidence of human listeriosis in the United States. Effectiveness of prevention efforts? The Listeriosis Study Group. JAMA. 273: 1118-22.

Vazquez-Boland J. A., et al., 2001. Listeria pathogenesis and molecular virulence determinants. Clin Microbiol Rev. 14: 584-640.

The entire contents of all references cited throughout this application, whether also listed in the list of cited references or not, are herein incorporated by reference in their entirety.

TABLE 1
Strains used in the multiplex PCR assay

| Strain | Species | Origin | Serovar |
|---|---|---|---|
| CLIP80722 | L. monocytogenes | Human | 1/2a |
| CLIP80730 | L. monocytogenes | Human | 1/2a |
| CLIP80841 | L. monocytogenes | Human | 1/2a |
| CLIP80853 | L. monocytogenes | Human | 1/2a |
| CLIP80888 | L. monocytogenes | Human | 1/2a |
| CLIP81049 | L. monocytogenes | Human | 1/2a |
| CLIP81209 | L. monocytogenes | Human | 1/2a |
| CLIP82243 | L. monocytogenes | Human | 1/2a |
| CLIP82823 | L. monocytogenes | Human | 1/2a |
| CLIP83088 | L. monocytogenes | Human | 1/2a |
| CLIP83240 | L. monocytogenes | Human | 1/2a |
| CLIP83283 | L. monocytogenes | Human | 1/2a |
| CLIP83434 | L. monocytogenes | Human | 1/2a |
| CLIP83641 | L. monocytogenes | Human | 1/2a |
| CLIP83792 | L. monocytogenes | Human | 1/2a |
| CLIP84054 | L. monocytogenes | Human | 1/2a |
| CLIP84084 | L. monocytogenes | Human | 1/2a |
| CLIP84191 | L. monocytogenes | Human | 1/2a |
| CLIP84244 | L. monocytogenes | Human | 1/2a |
| CLIP84248 | L. monocytogenes | Human | 1/2a |
| CLIP91992 | L. monocytogenes | Food | 1/2a |
| CLIP92009 | L. monocytogenes | Food | 1/2a |
| CLIP92062 | L. monocytogenes | Food | 1/2a |
| CLIP92121 | L. monocytogenes | Food | 1/2a |
| CLIP92130 | L. monocytogenes | Food | 1/2a |
| CLIP92204 | L. monocytogenes | Food | 1/2a |
| CLIP92216 | L. monocytogenes | Food | 1/2a |
| CLIP92227 | L. monocytogenes | Food | 1/2a |
| CLIP92294 | L. monocytogenes | Food | 1/2a |
| CLIP92306 | L. monocytogenes | Food | 1/2a |
| CLIP92312 | L. monocytogenes | Food | 1/2a |
| CLIP92325 | L. monocytogenes | Food | 1/2a |
| CLIP92331 | L. monocytogenes | Food | 1/2a |
| CLIP92344 | L. monocytogenes | Food | 1/2a |
| CLIP92360 | L. monocytogenes | Food | 1/2a |
| CLIP92439 | L. monocytogenes | Food | 1/2a |
| CLIP92443 | L. monocytogenes | Food | 1/2a |
| CLIP92450 | L. monocytogenes | Food | 1/2a |
| CLIP92456 | L. monocytogenes | Food | 1/2a |
| CLIP92461 | L. monocytogenes | Food | 1/2a |
| CLIP80663 | L. monocytogenes | Human | 1/2b |
| CLIP80723 | L. monocytogenes | Human | 1/2b |
| CLIP80731 | L. monocytogenes | Human | 1/2b |
| CLIP80914 | L. monocytogenes | Human | 1/2b |
| CLIP80970 | L. monocytogenes | Human | 1/2b |
| CLIP83180 | L. monocytogenes | Human | 1/2b |
| CLIP83293 | L. monocytogenes | Human | 1/2b |
| CLIP83482 | L. monocytogenes | Human | 1/2b |
| CLIP83775 | L. monocytogenes | Human | 1/2b |
| CLIP84085 | L. monocytogenes | Human | 1/2b |
| CLIP84294 | L. monocytogenes | Human | 1/2b |
| CLIP84320 | L. monocytogenes | Human | 1/2b |
| CLIP84515 | L. monocytogenes | Human | 1/2b |
| CLIP84522 | L. monocytogenes | Human | 1/2b |
| CLIP84688 | L. monocytogenes | Human | 1/2b |
| CLIP84809 | L. monocytogenes | Human | 1/2b |
| CLIP84899 | L. monocytogenes | Human | 1/2b |
| CLIP84974 | L. monocytogenes | Human | 1/2b |
| CLIP84981 | L. monocytogenes | Human | 1/2b |
| CLIP85102 | L. monocytogenes | Human | 1/2b |
| CLIP90640 | L. monocytogenes | Food | 1/2b |
| CLIP90861 | L. monocytogenes | Food | 1/2b |
| CLIP90979 | L. monocytogenes | Food | 1/2b |
| CLIP91313 | L. monocytogenes | Food | 1/2b |
| CLIP91357 | L. monocytogenes | Food | 1/2b |
| CLIP91428 | L. monocytogenes | Food | 1/2b |
| CLIP91580 | L. monocytogenes | Food | 1/2b |
| CLIP91592 | L. monocytogenes | Food | 1/2b |
| CLIP91865 | L. monocytogenes | Food | 1/2b |
| CLIP91959 | L. monocytogenes | Food | 1/2b |
| CLIP92008 | L. monocytogenes | Food | 1/2b |
| CLIP92052 | L. monocytogenes | Food | 1/2b |
| CLIP92182 | L. monocytogenes | Food | 1/2b |
| CLIP92192 | L. monocytogenes | Food | 1/2b |
| CLIP92198 | L. monocytogenes | Food | 1/2b |
| CLIP92205 | L. monocytogenes | Food | 1/2b |
| CLIP92390 | L. monocytogenes | Food | 1/2b |
| CLIP92429 | L. monocytogenes | Food | 1/2b |
| CLIP92441 | L. monocytogenes | Food | 1/2b |
| CLIP92445 | L. monocytogenes | Food | 1/2b |
| CLIP80665 | L. monocytogenes | Human | 1/2c |
| CLIP80669 | L. monocytogenes | Human | 1/2c |
| CLIP80724 | L. monocytogenes | Human | 1/2c |
| CLIP84516 | L. monocytogenes | Human | 1/2c |
| CLIP85078 | L. monocytogenes | Human | 1/2c |
| CLIP85305 | L. monocytogenes | Human | 1/2c |
| CLIP86391 | L. monocytogenes | Human | 1/2c |
| CLIP86600 | L. monocytogenes | Human | 1/2c |
| CLIP87640 | L. monocytogenes | Human | 1/2c |
| CLIP87923 | L. monocytogenes | Human | 1/2c |
| CLIP87968 | L. monocytogenes | Human | 1/2c |
| CLIP89425 | L. monocytogenes | Human | 1/2c |
| CLIP89496 | L. monocytogenes | Human | 1/2c |
| CLIP89796 | L. monocytogenes | Human | 1/2c |
| CLIP90481 | L. monocytogenes | Human | 1/2c |
| CLIP90606 | L. monocytogenes | Human | 1/2c |
| CLIP91334 | L. monocytogenes | Human | 1/2c |
| CLIP91363 | L. monocytogenes | Human | 1/2c |
| CLIP91383 | L. monocytogenes | Food | 1/2c |
| CLIP91450 | L. monocytogenes | Food | 1/2c |
| CLIP91588 | L. monocytogenes | Food | 1/2c |
| CLIP91767 | L. monocytogenes | Human | 1/2c |
| CLIP91855 | L. monocytogenes | Food | 1/2c |
| CLIP91875 | L. monocytogenes | Food | 1/2c |
| CLIP91929 | L. monocytogenes | Food | 1/2c |
| CLIP91945 | L. monocytogenes | Food | 1/2c |
| CLIP91980 | L. monocytogenes | Food | 1/2c |
| CLIP91983 | L. monocytogenes | Food | 1/2c |
| CLIP92007 | L. monocytogenes | Food | 1/2c |
| CLIP92077 | L. monocytogenes | Human | 1/2c |
| CLIP92080 | L. monocytogenes | Food | 1/2c |
| CLIP92124 | L. monocytogenes | Food | 1/2c |
| CLIP92221 | L. monocytogenes | Food | 1/2c |
| CLIP92322 | L. monocytogenes | Food | 1/2c |
| CLIP92353 | L. monocytogenes | Food | 1/2c |
| CLIP92359 | L. monocytogenes | Food | 1/2c |
| CLIP92363 | L. monocytogenes | Food | 1/2c |
| CLIP92447 | L. monocytogenes | Food | 1/2c |
| CLIP92468 | L. monocytogenes | Food | 1/2c |
| CLIP92470 | L. monocytogenes | Food | 1/2c |
| CLIP80650 | L. monocytogenes | Human | 4b |
| CLIP80671 | L. monocytogenes | Human | 4b |
| CLIP80701 | L. monocytogenes | Human | 4b |
| CLIP80721 | L. monocytogenes | Human | 4b |
| CLIP80751 | L. monocytogenes | Human | 4b |
| CLIP80759 | L. monocytogenes | Human | 4b |
| CLIP80760 | L. monocytogenes | Human | 4b |
| CLIP80776 | L. monocytogenes | Human | 4b |
| CLIP80849 | L. monocytogenes | Human | 4b |
| CLIP80879 | L. monocytogenes | Human | 4b |
| CLIP80882 | L. monocytogenes | Human | 4b |
| CLIP80963 | L. monocytogenes | Human | 4b |
| CLIP81101 | L. monocytogenes | Human | 4b |
| CLIP81117 | L. monocytogenes | Human | 4b |
| CLIP81146 | L. monocytogenes | Human | 4b |
| CLIP81252 | L. monocytogenes | Human | 4b |
| CLIP81438 | L. monocytogenes | Human | 4b |
| CLIP81594 | L. monocytogenes | Human | 4b |
| CLIP82016 | L. monocytogenes | Human | 4b |
| CLIP82169 | L. monocytogenes | Human | 4b |
| CLIP91390 | L. monocytogenes | Food | 4b |
| CLIP91555 | L. monocytogenes | Food | 4b |
| CLIP91590 | L. monocytogenes | Food | 4b |
| CLIP91686 | L. monocytogenes | Food | 4b |
| CLIP91740 | L. monocytogenes | Food | 4b |
| CLIP91781 | L. monocytogenes | Food | 4b |
| CLIP91806 | L. monocytogenes | Food | 4b |
| CLIP91863 | L. monocytogenes | Food | 4b |
| CLIP91899 | L. monocytogenes | Food | 4b |
| CLIP91909 | L. monocytogenes | Food | 4b |
| CLIP91921 | L. monocytogenes | Food | 4b |
| CLIP91942 | L. monocytogenes | Food | 4b |
| CLIP91950 | L. monocytogenes | Food | 4b |
| CLIP92085 | L. monocytogenes | Food | 4b |
| CLIP92209 | L. monocytogenes | Food | 4b |
| CLIP92278 | L. monocytogenes | Food | 4b |
| CLIP92288 | L. monocytogenes | Food | 4b |
| CLIP92334 | L. monocytogenes | Food | 4b |
| CLIP92406 | L. monocytogenes | Food | 4b |
| CLIP92449 | L. monocytogenes | Food | 4b |
| CLIP91375 | L. innocua | Food | ND |
| CLIP91443 | L. innocua | Food | 6b |
| CLIP91549 | L. innocua | Food | 6b |
| CLIP91879 | L. innocua | Food | 6b |
| CLIP91918 | L. innocua | Food | 6a |
| CLIP91922 | L. innocua | Food | 6b |
| CLIP92006 | L. innocua | Food | 6b |
| CLIP92186 | L. innocua | Food | 6b |
| CLIP92218 | L. innocua | Food | 6b |
| CLIP92310 | L. innocua | — | — |
| CLIP 74914 | L. ivanovii/ivanovii | — | 5 |
| CLIP 88111 | L. ivanovii/ivanovii | Food | 5 |
| CLIP 86784 | L. ivanovii/ivanovii | Food | 5 |
| PAM55 | L. ivanovii/ivanovii | — | 5 |
| CLIP 6645 | L. ivanovii/Iondoniensis | — | 5 |
| CLIP 12065 | L. ivanovii/Iondoniensis | — | 5 |
| CLIP 73021 | L. seeligeri | — | 1/2b |
| CLIP 86579 | L. seeligeri | — | 1/2b |
| CLIP 87073 | L. welshimeri | — | 4c |
| CLIP 87973 | L. welshimeri | — | 6a |

CLIP: Strain number in the Listeria Culture collection of the National Reference Center for Listeria, Institut Pasteur
ND: Non-determined serovar

TABLE 2
Strains Used in DNA/DNA Macroarray Hybridization Analysis

| Strain | Species | Origin | Serovar | Year | Country |
|---|---|---|---|---|---|
| CLIP 61673 | L. monocytogenes | Human, | 7 | 1995 | France |
| | | sporadic | | | |
| CLIP 74917 | L. monocytogenes | * | 7 | — | — |
| CLIP 42636/T23 | L. monocytogenes | Human, | 1/2a | — | UK |
| | | epidemic | | | |
| CLIP 46664/T20 | L. monocytogenes | Food | 1/2a | — | USA |
| CLIP 46682/T56 | L. monocytogenes | Human, | 1/2a | — | USA |
| | | sporadic | | | |
| CLIP42663/T77 | L. monocytogenes | Human, | 1/2a | — | USA |
| | | sporadic | | | |
| CLIP 46661/T14 | L. monocytogenes | Human, | 1/2a | — | USA |
| | | sporadic | | | |
| CLIP 42651/T53 | L. monocytogenes | Animal, | 1/2a | — | UK |
| | | sporadic | | | |
| CLIP 9429 | L. monocytogenes | Human, | 1/2a | 1988 | France |
| | | sporadic | | | |
| CLIP 74902/ | L. monocytogenes | * | 1/2a | — | — |
| ATCC35152 | | | | | |
| CLIP 61523 | L. monocytogenes | Food | 1/2a | 1994 | France |
| CLIP 61678 | L. monocytogenes | Food | 1/2a | — | France |
| CLIP 61634 | L. monocytogenes | Human, | 1/2a | 1995 | France |
| | | sporadic | | | |
| CLIP 61839 | L. monocytogenes | Food | 1/2a | — | France |
| CLIP 61868 | L. monocytogenes | Human, | 1/2a | 1995 | France |
| | | sporadic | | | |
| CLIP 87880 | L. monocytogenes | Food | 1/2a | 1992 | France |
| CLIP 87881 | L. monocytogenes | Food | 1/2a | 1993 | France |
| | | Environmental | | | |
| CLIP 87884 | L. monocytogenes | Food | 1/2a | 1992 | France |
| | | Environmental | | | |
| CLIP 87890 | L. monocytogenes | Food | 1/2a | 1992 | France |
| CLIP 69566 | L. monocytogenes | Environmental | 1/2a | — | France |
| CLIP 80559 | L. monocytogenes | Environmental | 1/2a | — | — |
| CLIP 60368 | L. monocytogenes | Environmental | 1/2a | 1994 | France |
| CLIP 14843 | L. monocytogenes | Environmental | 1/2a | — | France |
| CLIP 89461 | L. monocytogenes | Environmental | 1/2a | — | Danemark |
| CLIP 89458 | L. monocytogenes | Food | 1/2a | — | Danemark |
| CLIP 87908 | L. monocytogenes | — | 1/2a | — | — |
| CLIP EGD | L. monocytogenes | — | 1/2a | — | — |
| CLIP 46686/T64 | L. monocytogenes | Human, | 1/2b | — | USA |
| | | sporadic | | | |
| CLIP 42655/T61 | L. monocytogenes | Food | 1/2b | — | USA |
| CLIP 989 | L. monocytogenes | Human, | 1/2b | 1981 | USA |
| | | sporadic | | | |
| CLIP 3559 | L. monocytogenes | Human, | 1/2b | — | Argentina |
| | | sporadic | | | |
| CLIP 87878 | L. monocytogenes | Food | 1/2b | 1993 | France |
| | | Environmental | | | |
| CLIP 87879 | L. monocytogenes | Food | 1/2b | 1992 | France |
| CLIP 87888 | L. monocytogenes | Food | 1/2b | 1992 | France |
| CLIP 87897 | L. monocytogenes | Food | 1/2b | 1999 | France |
| | | Environmental | | | |
| CLIP 87909 | L. monocytogenes | Food | 1/2b | 1999 | France |
| CLIP 87910 | L. monocytogenes | Food | 1/2b | 1993 | France |
| | | Environmental | | | |
| CLIP 70086 | L. monocytogenes | Environmental | 1/2b | 1995 | Argentina |
| CLIP 70848 | L. monocytogenes | Environmental | 1/2b | 1996 | Espagne |
| CLIP 76278 | L. monocytogenes | Environmental | 1/2b | 1998 | France |
| CLIP 14842 | L. monocytogenes | Environmental | 1/2b | — | France |
| CLIP 89460 | L. monocytogenes | Environmental | 1/2b | — | Danemark |
| CLIP 46694/T80 | L. monocytogenes | Human, | 1/2c | — | UK |
| | | sporadic | | | |
| CLIP 42653/T57 | L. monocytogenes | Human, | 1/2c | — | UK |
| | | sporadic | | | |
| CLIP 42972/T8 | L. monocytogenes | Human, | 1/2c | — | UK |
| | | sporadic | | | |
| CLIP 2710 | L. monocytogenes | Human, | 1/2c | — | Czech |
| | | sporadic | | | Republic |
| CLIP 11550 | L. monocytogenes | Food | 1/2c | — | France |
| CLIP 87891 | L. monocytogenes | Food | 1/2c | — | — |
| | | Environmental | | | |
| CLIP 87892 | L. monocytogenes | Food | 1/2c | — | — |
| | | Environmental | | | |
| CLIP 87893 | L. monocytogenes | Food | 1/2c | — | — |
| | | Environmental | | | |
| CLIP LO028 | L. monocytogenes | — | 1/2c | — | — |
| CLIP 86436 | L. monocytogenes | Food | 3a | 2000 | France |
| CLIP 74905/ | L. monocytogenes | * | 3a | — | — |
| ATCC19113 | | | | | |
| CLIP 8053 | L. monocytogenes | Environmental | 3a | 1987 | Finland |
| CLIP 74906 | L. monocytogenes | * | 3b | — | — |
| CLIP 87900 | L. monocytogenes | Food | 3b | — | |
| | | Environmental | | | |
| CLIP 3558 | L. monocytogenes | Animal | 3b | — | Argentina |
| CLIP 11962 | L. monocytogenes | Food | 3c | — | France |
| CLIP 85412 | L. monocytogenes | Food | 3c | 2000 | France |
| CLIP 74907 | L. monocytogenes | * | 3c | — | — |
| CLIP 74908/ | L. monocytogenes | * | 4a | — | — |
| ATCC19114 | | | | | |
| CLIP 78025 | L. monocytogenes | — | 4a | — | Germany |
| CLIP 71988 | L. monocytogenes | — | 4a | — | — |
| CLIP 73722 | L. monocytogenes | Human, | 4b | 1997 | France |
| | | sporadic | | | |
| CLIP 46684/T60 | L. monocytogenes | Environmental | 4b | — | Switzerland |
| CLIP 46679/T50 | L. monocytogenes | Food, epidemic | 4b | — | Canada |
| CLIP 42635/T21 | L. monocytogenes | Human, | 4b | — | Switzerland |
| | | epidemic | | | |
| CLIP 42652/T55 | L. monocytogenes | Food, epidemic | 4b | — | Switzerland |
| CLIP 42639/T29 | L. monocytogenes | Human, | 4b | — | USA |
| | | epidemic | | | |
| CLIP 42646/T43 | L. monocytogenes | Food, epidemic | 4b | — | USA |
| CLIP 4598 | L. monocytogenes | Food | 4b | — | Switzerland |
| CLIP 2698 | L. monocytogenes | Human | 4b | — | Czech |
| | | | | | Republic |
| CLIP 8715 | L. monocytogenes | Human | 4b | 1988 | Suisse |
| CLIP 74910/ATCC | L. monocytogenes | * | 4b | — | — |
| 19115 | | | | | |
| CLIP 22573 | L. monocytogenes | Human, | 4b | 1992 | France |
| | | epidemic | | | |
| CLIP 27993 | L. monocytogenes | Food | 4b | 1992 | France |
| CLIP 87889 | L. monocytogenes | Food | 4b | — | — |
| | | Environmental | | | |
| CLIP 87896 | L. monocytogenes | Food | 4b | — | — |
| | | Environmental | | | |
| CLIP 87911 | L. monocytogenes | Food | 4b | — | — |
| CLIP 87915 | L. monocytogenes | Food | 4b | — | — |
| CLIP 87917 | L. monocytogenes | Food | 4b | — | — |
| CLIP 72783 | L. monocytogenes | Environmental | 4b | 1996 | Switzerland |
| CLIP 60383 | L. monocytogenes | Environmental | 4b | 1994 | France |
| CLIP 87691 | L. monocytogenes | Environmental | 4b | — | Portugal |
| CLIP 80459 | L. monocytogenes | Human, | 4b | 1999 | France |
| | | epidemic | | | |
| CLIP 74911/ | L. monocytogenes | * | 4c | — | — |
| ATCC19116 | | | | | |
| CLIP 86309 | L. monocytogenes | Food | 4c | — | France |
| CLIP 81065 | L. monocytogenes | Animal | 4c | 2000 | Switzerland |
| CLIP 74912/ | L. monocytogenes | * | 4d | — | — |
| ATCC19117 | | | | | |
| CLIP 79619 | L. monocytogenes | Animal | 4d | 1999 | Switzerland |
| CLIP 74913/ | L. monocytogenes | * | 4e | — | — |
| ATCC19118 | | | | | |
| CLIP 78003 | L. monocytogenes | — | 4e | — | Germany |
| CLIP 79455 | L. monocytogenes | — | 4e | — | — |
| CLIP 71990 | L. innocua | — | 6b | — | — |
| CLIP 71989 | L. innocua | — | 6b | — | — |
| CLIP 74915/ | L. innocua | * | 6a | — | — |
| ATCC33090 | | | | | |
| CLIP 74916/ | L. innocua | * | 6b | — | — |
| ATCC33091 | | | | | |
| CLIP 88566 | L. innocua | Food | 6a | — | France |
| CLIP 88307 | L. innocua | Food | 6b | 2001 | France |
| CLIP 86490 | L. innocua | Food | ND | — | Maroc |
| CLIP 11262 | L. innocua | Food | 6a | 1988 | Maroc |
| CLIP 74914/ | L. ivanovii/ivanovii | * | 5 | — | — |
| ATCC19119 | | | | | |
| CLIP 88111 | L. ivanovii/ivanovii | Food | 5 | 2001 | France |
| CLIP 86784 | L. ivanovii/ivanovii | Food | 5 | — | France |
| PAM55 | L. ivanovii/ivanovii | — | 5 | — | — |
| CLIP 6645 | L. | — | 5 | — | Switzerland |
| | ivanovii/londoniensis | | | | |
| CLIP 12065 | L. | Animal | 5 | — | Belgium |
| | ivanovii/londoniensis | | | | |
| CLIP 73021/ | L. seeligeri | * | 1/2b | — | — |
| ATCC35967 | | | | | |
| CLIP 86579 | L. seeligeri | — | 1/2b | — | — |
| CLIP 87073 | L. welshimeri | Environmental | 4c | — | France |
| CLIP 87973 | L. welshimeri | — | 6a | — | France |
| CLIP 73019 | L. grayi | — | — | — | — |
| CLIP 12515 | L. grayi spp. | — | — | — | — |
| | murrayi | | | | |

CLIP: Strain number in the Listeria Culture collection of the National Reference Center for Listeria, Institut Pasteur.
ATCC/Strain number in the American type Culture Collection,
* Indicates reference strains for serotyping.
T: Numbers according to the strain set of the international WHO multicenter typing study.

TABLE 3

ORF1025 (SEQ ID NO:1)
GGATACGGGAGTGTGGCGCTCGTCATGAAGATAAACAAAATTATCGAATA
TATTGTAGTAGCAATCATCGTTTTAGGTTGGACTTACTTCATTGTTATTA
AAGAAATTCTCCCTAAAAATATAGGATTTATTTCATTAGTAATTTTATCA
ATAATTCTTTATTTTGTAGGCAAAAGGTTGAATGGGAAACATAGCAGATA
A
ORF104 (SEQ ID NO:2)
GAGAAAAGTATGAAAAAAATACTTGGCGGAATCTTAATTATTTTTGTAAT
AGGCCTGTTTGCTTGGCGCGTATATGATGTAAAAGCTAATTCTTTTTCAT
ATGAAAATAAGGCACATGCTGAACAGGAAAAATTTCAACTTGGAAGCTCA
ACAATCAGTGCTGGAAAAGCATTTGTTGTAAGTGATGCAGACTTAAATAA
ATATGTGACAAAAGATTATTTTAAGCAGGAAAATAAGACTCTTTTGCTCG
TTCAATTAGAATCTACAGAAAAAGATATAAGAATTTCTGATTTTCAGTTA
GGGTATAAAGAATTTGTAACGTTGTCAGATACTTCGGCAGCAAGTTATGA
ATTTGAAGATGGGGTGTATAAAATGGTGTTAGGTTTTAATATACCTAAAG
AATTACTAGCAACTAACAAGACATTCACATTAGTTACTCCGAGTAAATAT
TGGAAGAACGGAGCAAGGGATGTTGTGGAAATCAGTTTATAA
ORF105 (SEQ ID NO:3)
ACTAAACTAGGGAGAATATCGATGAATTATATAAAAGTTAATCATTTAAC
AAAAGTAATCAACAACAACACTGTACTAGATGATATTAATTTTGAATTAA
AGCAAGGTGGGATTTATTCGTTCATAGGTCATAATGGCTCTGGAAAAACG
ATGCTTTTTAGGGCGTTATGCGGTTTTATTGCGCCAACAAGTGGTGAAGT
TACAATTAATGGCATTAGTATTAGCAAAACTAGATCTTTCCCAGAAAAGA
CCGGAATTATTATTGAAACGCCAGGCTTTTTAGCAAATTATACTGGTTAT
AAAAATTTGGAATACTTAGCTTCTATTAGAAATCAAATAGGTGAAAAGGA
AATACTTGCTGCGTTAGAACAAGTTGGACTTTCTGGAAAAGAGAACTTGA
AAGTGAAAAAATATTCGCTTGGAATGAGACAACGGCTTGGTATTGCTCAA
GCTATTATGGAAGACCCAGATTTATTAATATTTGACGAACCAACAAATTC
GTTAGATAAAGCTGGCAGTCAGAGTTTTATTGACTTGATTTTAGACTTAA
AAGAAAAAGGAAAAACGATTTTGTTAGCAAGCCATCATATTGCTGATATT
GATGGTATATCAGATGAAATATTTGAAATGGAAGCAGGTCAGATAATAAA
TAGGAGAAAAGTATGA
ORF1063 (SEQ ID NO:4)
ATTGGAGGAGATAGGATGAAAAATCTATTTAGACTATTCTTGGTATTTAG
CATAGTTATTATCGGGGTTGTTTCTTTTCAAGCGATAGATGCAAGTGCAA
ATGAGACAGATGTATATCCATTACCAGCCAGAATAATTGATGTTTTCCCA
GATGAAAATTTAGCAGAAGATATGGTGGAGAACTTTGGTAAAAAAGATGT
GACAGATGTAATTACCCAAGATGATGTGGATGCGGTGACATCTCTAGGGT
TGGGTTATTTTACCAATTACCTCACAGATGAAGATTTACAAATGCTAGGA
AATGCTTATTTTACCAATGTAAATAATATTATGATTTATCCTACACAAAC
CATGTTTACTGGCTTTCCTGATTTACCAACCTTGCCCAAATTAGATACTT
TGAGAGCAGAAGGAAATCTATCAAGCGAGGTACTACCTGAAAACATTACA
GTTCCCGATTATCAAAACTACCCAGAATTAAAGTATCTTGATCTTAGTAA
TCGAACTATTGTTGGGGGCTTACCTAACTTTTCTAATATTCCTAAATTAG
AAACCTTGTTGATGAGCAGTTGTGGACTAGCTTCGGAAGACGCTCCAGAT
TTTACAAACTTGAAAAATTTGCAGAAAGTAAATTTTCAAACGAACCAATT
TAGAACAGAAATGACGGATTTCACTCATTTGGACAGTTTGGTAAGTATGG
ATTTAAGTTATAATTATTTAAATGTGTTGCCGCCTACTATTGTGGATAAA
GTAATCGTACTAGGTCAAATCGGAACTTTGCCAGATCAAAATGTTGTTTT
TGGTGAGGATACAAATATCACGCTACCAGTTTATACGCAACTAGATGATT
TAGGTAGAATTAGTGGTTTTCAAGAAGTTTGGATTCGTGATTCAAATGAG
AAGGAAATTTACAACGTGGCAAAAGTGGATTACGACGAAGTGACGAAGCA
GATTATTGTGCCAACAAATAACCTTGATAAAGGTGAATACACTATTGGAA
TTGATTTTAATGGAATAGAGCCTTATATAGAAGAAGGCGAAGTTATGAAT
TACTCAGTAAAAATAACCATTAATTAA
ORF1227 (SEQ ID NO:5)
AAAATAAAAGGATATATCAATGTGAATATAACTAAAAACGGATTATATAT
AATTATTGGGGCAAATGGAGTAGGGAAAACAACATTAGCTAAGAAAATAC
TTCAAGCTAACAGAAGTATTAGTTGCATGATGAAGCAAGATGATAATCAA
ATATTGGAGTATGAGACGGTTTTAACCAATATTAGTATGAACGAAATCGC
AGAAAAAACGGTTATTAATTTTCTGGAAGAACATCAATTAGACTATTTAA
TAACTAAAAAAAGTAAATTTCTTAGTGGGGGAGAAAAAAGATTAGTTAAC
CTTCTAAGAGCTATTTTGTCAAATCAAGAAGTATTGATATTAGATGAGCC
ATCAAATGACTTAGATATTGATGTATTTGAGAAGGCAAAACAAATTATTT
ATCAAGCGGCAAAATCTAAAATAATACTATTAATTACGCATGATGATCGT
TTTACCGAATATGATAAAAAAATAGAAATAATGAAGAATCAAAGTTACGA
AGCAGATAGTTTCAGCTTTAATAAAGAAAGTTCGAAAGCGCGCATTATCA
AAATTAAACCTAGAAGGACCTATTTCTTATATATCTTCTATTTAATTTGT
ATGATGATTTTCGCTATTTTTTTAGTGATTTTACTGAAAACAAATGCTGA
AGAAACAAGTCCTTCTAACGAAAAAGGTACATATCAACTTGCTACAATAT
ACAGCACTAATGCTTCTTCTTATGACAATAATGAAGCAATTAATACGATG
CTTATACAATCAGCAACTAAATTTAATAAAGCTAAGTTCTTCACAGAAGA
AACACGCATTAATGAAGACGAGTATTATGAAGAAGCGATTAATTTGAAAA
AAGATACTTATCAAGAGTTAATATATTTAGAATTATATGATCCAAAAACA
GAAGAATTTATCAATATTAAAGCTGTAATGATGGAAGCTTTACGTGGAGA
TTTAAAGTTGAACGCAGAGACAGAATTCATTAGTAATGACGAAAATTACT
ATAAAAATAGTGATAGTCCATCTTTCCACGTGCCTAAAAGTTTAACTTTA
ACAGAAATTAAAAAAGCTAAAATTAAGCAGCTAGGATTTGAACTTCATTA
TAGTAATACTTTGCAATCAAATCAGGTAGAAATAGAATTTAATCCTAGCG
TGTATGCGCAAATTTTGAAAAAAGTAAATCAACAAGATGTTTTAATTACA
GAAGCATACGTGCAATTAAAAGCTCAAGAATCATTTTATGATTTCTTAGC
AGAAAATAAACTATATGCTAAAAAAATATTTATTAAAGGATATGAACCAG
AGTTATTAAATGCTGAGGTTAATCAATACAGTAATGCAGTTATGCTGATA
AAAAAAGTAGCACTTCTTATATGCTTACTACTCTTAGTATTATTAATTTT
ACTAATTATGTATGAGGTTAGTTATAAAAATAGTTATAGTACATTGACTT
ATTATGGCTATAATGAAAAAGAGCTACTTCAATTCAGAAAAAAAACGTAT
CTTATTACAAACTTTAAAATCTTTTCGGTTATTAGTACTGTTATTTTTCT
TTTAATTATGTGGAGTATTGTTCATTCGGTACTTATTACCGCCATAATAG
GTGTAGTAATGAAATTTTTCTTTTTTGCCTACATCGTCATACCATTAATA
ATTAAAAACAACATTAGAAAGGCGATTATATGA
ORF1228 (SEQ ID NO:6)
AAAGGCGATTATATGAATAAAATAAATAAATTTAGCATTTTTTCTATCAC
TAAACCAGGTATTTACACAATTACAGGTAGTAACGGAAGCGGAAAAACAA
CCTTTATTGAAAATGAATTAAAAAATAACACAAATAAAGTAAAAGATGTA
GCTTACTTTGCGCAAAAAAATTGGAAATATAAAACTAATGTAGAGAAATA
TTTACACTTCCCAAAAACTAATCCGAGCTTAGTTCAAAAGTATTGCGAGT
TGTTTTCTGTAGATAACTACTACTTAGAAAAAGATATCCAATTATTAAGC
GGCGGAGAATTTGTCAAAGTAGAATTAGTGAGAACATTAGCTTTGGACTC
TTCTATAATTATTCTAGATGAACCTACAAATAATCTAGATAACAAGTCCT
CGGAGATACTTGCTAATATTTTAAGTGAATTAGCAAAAACGAAAATTATT
TACTTAGTTAGTCATGATACTCGATTGGAGCATTTTTTTGATAAAACTAT
TTTTGTAGATAAAGATAGGATAGAAGTATCGTCTAATGTTGAAATAGAGC
AGAACGAGATTCAAGTTAATAGTAAAAGAGTTGTTTCAAATGGAAGAATT
TTAAAATATTTACTTAGTTCAAAATTTAACTTCTTGATGTTTGCGTTTAT
TATAGTACTAACGATCTTATTAACCAATATTACCTCCACTATTATTTTAC
GCTCAGTTCCAATAGAAGAGAATTTAACTAGTGATTATAACTTTGAGTTG
ATGGATATTGCGGAGAATTATTCTAGATATTTTAATATTGAAATGACGGA
AAGTGAAATTGAAGCTGAATTTCAGGAACCTAATCATCTCACTACAAATG
AGTTGATTGAATTACAAGACAAAGACTATATCAAACAAATTTATGTTGTA
GATGAAAGCTATATAAATGAATTTGTTTTGGATAATTCCAAGTTTGAAGT
TTTGGCTCTTCCGGAAATAATTACAGACTCGCCTAACTATGTAAATGCTT
TTCCTGTTACTAAAATGCACTTAACCAAAGGACGTTTTCCAAAAGACGAT
GCTAAAGAAATCGCGTTATCTTTTGCTCAACTGAAAAAATTTTTCCATGA
TGATATTAGTGAAGAATCAGCTATAGGTAATAAATTAGAATTTGAAGATG
AGTTGTATGAAATAGTCGGTATCGTTAACTCTCCAGTAGCAGCGATCTCT
TATTCGAAACAAGTGAGTAAAGGAACTGTTGAGGTTGAGGATAAGACTTC
TGAGAAATTAAATAATATATTGTTAAAGCTTGAGAAAGAAAATTATGATA
ACCCTAATTTTTCTATTATTTCCATTAAACTAGCTAGTAAAAACCAACAT
GAACTCTTAAACTATTTAAAAGTTCATGGACCAAGCTACCAATATGCCTC
AAATTATGTGGATAGTATTTCGCAAGTAGCTTTTTATAAACAAAATTTAG
CAAAAATACTTCTTATATCAGTAATTTTTTCTCTTATAGTATCTGTGTTG
ATTTTTATTTTTGGAAGAAAATCATTTAGTTTAATTAATGGCTTTTTAAA
CGATATGTCTAATTTGAATTTTACACCGAGAAAGAATAAACGGTTTATTT
ATGTAATAATGATATTAGATTTTTTGTTGAGTATGCCTGCGTGCTTGTTA
GTAAGTCGAGTTATTATTGGGGATAATATAGGTATGCTAATGATTCTTCC
GACACTGGGGGGTTCAGCTATCATATTTATGCTCACATTACTATTAATGA
GTTACCGGGATAAGAAGAATGATTTTAGAAATTTATAA
ORF1272 (SEQ ID NO:7)
AGTAGACATGTCATTAATTGTAATTTTAGGAATTTGTTTTATTTTATTAA
ATCCAATAAAACTAGCAATAAGGTTTCAGAATTCCAATATTTGTTGTATA
TTTTTTGTTTTCAATGGTTAATTCTCTTGCTTATAAGATTCTTTTATTTT
AGCTTGTTTTATTTTTTTCAAAATAAAGTTAGTTTCACTCATCAAACTAT
AACTAAAGCATATGTTTTATTATTTGTTTTGTGGATTGCTGTTTTTATTG
TTTGTTGTTTTCGTTTTAGAAAAAAATTAAGAAAGGGTGATTTTAGAAAA
GATTCTGTATTACAAAATAAACGGGGTAACTTAGGATTGAATTTAAGCAA
AAAAACATACATATCAATTGGAGCATTTTTTGCATTTATATTACTAGCTC
AACTTATTGGAGGAGTAATGATATATGTATTCTTTATCTCTATAGCGGTT
TTATGTATGGCATTATCTTTTCTAGGATTAGTGATCTTTCCTGAACAAAT
TTTCACTGCTTATTGTAATTTTAAAGAATTTCATATCGAAGAATAG
ORF1384 (SEQ ID NO:8)
AGGAACGGCTTTAATGTTGTGGAGCCGGACTTTGCTTATCAATTTGAAAC
GAGAGAAGAGATGGTTGAAAAAAGCAGTTGGTATGCGGAAGATTTTGATA
TTGTTGCTAGTTTGGAAGAAAAGTACTTAGCTGGATTCTATCAAAATCAA
GTCTATGGTTACGTAACTCCAGTCGAGTTTCAAGAGGAGGAAATGAAAAG
AGCGATTCCAAGCGAAATGTTTGTTCCTTTGAAAGGCTTAGAAGTTTCTG
AGTTGGCGGATTGGGCAGATGGTTTTCCTCCTGAATTGCAAGAACGGTAT
AATGAGCTAGCTGAATCAGTGGAAGAGTAA
ORF1385 (SEQ ID NO:9)
AAAGGAGATCATCAAATGAGTATTACAGAAGAAGAATTTGAAGAACAGGT
TTCGGAGTTGTTTATTAATTATTTAGAAAAATGTACGCCTGAAGAGATTC
ATCAAGTGGTTGTAGAATGGAATTTTGATAATCCGAAGAAACCGATTCAT
TGGATTGCTAATTCGACGAAAACGGATAAAGGAACGGCTTTAATGTTGTG
GAGCCGGACTTTGCTTATCAATTTGAAACGAGAGAAGAGATGGTTGAAAA
AAGCAGTTGGTATGCGGAAGATTTTGATATTGTTGCTAGTTTGGAAGAAA
AGTACTTAG
ORF1386 (SEQ ID NO:10)
TCCAGCACAATAATTTTAAAGGAGCGTAACTTTATGAACGAATTTCCAGT
TGTATATGAACCAGAGTATATGACCATTTATTTACTTGTTATCATCGGTG
TCTTTTCTACAACATTGGCTTTATCGCTTGAAAAATATAAGTTCACTTTA
GCGGGAAAAGCGTTGATTGGGATGTGTTTTATTTCTCTAGCTGTGTTATT
TATATATGTTTTAGAGTTAGCATATCTTTCAGATATAGCTGATTTGTTAT
TTCATACAATCGGTGTTTGGGAAGTAGTGCTTTTCACACATCCATATATT
TTCTTAATACTTGCAATCTTGCTCGGCGGGGAAAAGAAACCGCCACATGT
ACCCAAACTTAAAAAATAA
ORF1387 (SEQ ID NO:11)
ATTTTACTACCATATTTATACTCAACCAAAAAAGAAGGAAAAAACATGTT
ATCCGATATCACAATTACAATTCCTAACAAAACAGCTCAAACAAAATACG
ATAATCTAGCAAAAAACATGTCTTTTAAATCAAAAGAGGATTTGGAGTCA
TTGCTTGATTTGATCAAGATTTTGTATATAAACAATTATTATGAAGAGGC
GCTTTTGTGTTGCCGACTAACGAACGACGTAGAATTTGATAATGATTTTG
ATGTTTGGACATTTATTCATAGGATTTGGATGTTTGAAATGCAAATACTT
GTGCATTTTGGGGAAAAAGAAGAGGCCGAAGAAATTGCCACTAAAATAGA
GGCGCATTTTAAAATTCCAATGAAAATATGGAATACTCCTGAAAAAAGAT
ACGCGCAGTATAAAAAGAATCGTTCTCGGATTGTTCTTAAGGATCTTAGC
TATGAAGAAAAAATCAATAGCACACCTAGAGGAGAATCTGGAACATTTGA
TTGGAAATTTGTCGCATTATCAAGTCTTATCAAACCAATTACTTTGAACA
ATATGACTGGTGTTTCGGTAGAAGAAGCAGAAGAAATATTTAGACAATAT
TCAGCAGAGTTACAACAAACGAAAAAGTATGGAGTTTCGTTATAA
ORF1388 (SEQ ID NO:12)
TTGTTATATAGTTTCCAAAAATTTGATTGGCTTATTGGTTATTCTGATGA
TGGCGATTATGTTACTTGCGTAGATAATGGACTGTTCTTTTCTATAGATA
AAATGGTCCTTCTAGATAATGATATAAACGTACATCTATTGCCAGTACAG
GCTCACTAA
ORF1389 (SEQ ID NO:13)
AAAAAAATAGAAAATAAAATAATCTCTAAATTTATCAATCTGGAAGATGG
TTTTGATTTTAATTTGCATCGAGCGTATTTGAGATATAATCATAAATTAA
GATGTATTGGAATTAGTTATCAAAAAAATGATTGGTGGGAATGGTTTAAA
TTAGTTCCAAGCATTGTAGGACAAACAGCACCTGTATATGTTGTTTTTAC
TGGTGATAATTATACATGGATCTATACAGGTTATTTAGAGGCGTTAATGA
AAATTTTATATTATGGCAGTCCTATAGGTGAAGGTGATTGTTATATAGTT
TCCAAAAATTTGATTGGCTTATTGGTTATTCTGATGATGGCGATTATGTT
ACTTGCGTAG
ORF1390 (SEQ ID NO:14)
TTATATAAAAGGATATTCTTAAAGCATGATATAATTATTTTAGTATTAAA
AATTTATTTAGGAGGAATTAAATTGAGATTAACTAGATTAAAAATTACGG
ATTTACATAGTTCTTATAGTTATGACATCGAATTTAATAAAGACATCACT
TTTTTTTACGGGACTAATGGTAGCGGAAAAACAACAATATTAAATATTCT
TTCTAGTATTGTAACAGGAAAAATTTATCAACTATTCACTTATGAATTTA
GTTCAATTACTCTACACTATTGTTTTGAAAGAACAAAAAATAAAGAACAA
AAAATTGAAATACTTAGAACTACACCCCTATGTATTGAAGTTACATTCAA
TGGTCAGAGATATATTTTAGAAAAATTTAATGAATCTTTGAGTAATTATC
GTCAATCTATAAACAGAAATCTAGAAAAAGACTTTTTTGGTGAAAATAAA
TTTTTAAAAGAAATAGCAGATATGTTTAACTATGTGTACCTTCCACTAAA
TAGACATATTTCATTAGATAATAATATGTTATATGATAATAGATTACATC
GTGAAAGAACAATAAATGCAATGATGCATGGAGTTGAGGAGGATGAAAAT
GATTACTATATGGAAAGAGATAAGTCGATGAAGAAGGTTCAATATTTAAT
AAAGACAAAAGTAATGAGGAATAATACTAGAATCAACAGACTAAATGATA
ACTTCAGAAATCAAATACTAAGATCCTCTATAGATATACACAAGCTTACT
TTTAGTATAGAAGAAATATTTAATGAGGTTAAAAACTTAAAAGTAGAAGA
AATCAAAGATATCAAAACATCTTATATAAACATATTGAAAAATTTGAATC
AAATAACCCAAATTGAAGAAAAAAATTATGTTAATTTTTTTGATAATTAT
ATAAGAGAAATTAAAGAGAATGAAAATATTGAGGATAATTCAGTTTCAAT
TCAGTTGATTTTAAAATATAATGAAATAGCAAAAATAAAAAATATAGTAA
AACTAGCCGAGGATATGGAAACTAAAAAAGCAAAAGTAGTTGAATCTGTT
AATTTATTTTGTGAAACTATTAACAGTTTCATATCAACCGATGGTACAGA
AAAAAAAGAAATATTAATTGATGAAAACGGAGGTATATATTTACAAAATG
TAATTGAAAAAAAACAATTAAGTATATATAAACTTTCTTCTGGGGAAAAG
CAAATTGTAATTTTTTATGCAAACTTAATTTTTGGAGTAAATGAAAATAA
AAGAGGTATATTTATTGTTGATGAACCTGAACTATCGTTACACTTATCTT
GGCAAAAATTATTTATTGAAAAAACTATAGAATTAAATAAAAATATACAA
TTTATATTTGCTACTCATTCGCCAGAAATAATAGGTAAATATTATAATAA
AACATTCGAGTTAAAGAAAAAGATAGATAGATAG
ORF1391 (SEQ ID NO:15)
GAGAAACTCAATAATATGAATCAATTACAATACTCTGAAGATGCTTTAAA
AAATAGGTATCTCTTTTTTAAAGAAATAAATGAAATAAATATTTTTGTTG
AAGATAAAGGAAAAGAATATGAATATGAGGAAATATTGTCTAAAGTTTTT
GCTGATGAATATCATATACAGACGATATATGCACTGGGGGGTAAACCACA
AGTTTTATCCGCATTTAGAGACTGTAAAGAACATGTTTCTAGCAATAATA
ATATAAAAAACATATATCTTTTAGATGGTGATTTTGATCAATATTTGGAT
AATATAGTAATGGAATCACATCCACATATTATTTATTTGCAATCATATAA
TATTGAAAATTATTTTTTGAATGAAGAATCGGTAGTAACTTTTATGCAAG
GAAAAATGAAAATGCTGAAGAATGATGTAAGGACTAAAATAGCTTATTCT
GAATGGCTTTCTAATACATTATACAATATAGAAAACATTTTTATTTTATA
TTGTATAATTCAAGAAAAGGAATTAGGGATTCCTAATGTAGGGGATTCTG
AATATAAAGTAATTAATATGATCGATGGATCTATCAATATGACGAGATAT
GAGGATTTGAAAAAGCAAATAAGTAATTGTACAGTAAACATAGATTTAGT
AGAGCAAGAAATGAAAGCAAAAATTCACAATATAAATCCTAATGTGTTTG
ATTTGATTTGTGGTAAACATTTGATTTGTAGTTTGTACCGTCATCTGTTA
AAAATAACTAAAGGTTTCAGCTATGATGAATTTAGATGGCATTTAGTTCA
AAATGTTGAAGTATCTAGACTTAATTTTATAAAGGAAAGAGTTGCTAATT
TGTAA
ORF1400 (SEQ ID NO:16)
ATGAACAGTGATTCAAATAATAACATCAATAAGAATCGAGAAGCGGGAAT
AAGTATTATTATTCCATTATATAATGTGGAGGAAGTAATCCTGGAAACGC
TCGAAAGCATCCATGAGCAAACATTTGACATGTACGAAGTTTTATTAATT
GACGATGGTTCAACGGATAAAACAATAGAAATGGTGACAGAATATATATC
AGATAAGCCAAAATTTCAGTTGCATACTCAACCAAATGGTGGACCAGCTT
CGGCTAGGAATTATGGGTTGCGTCTTGCGAATAGAATGTATATTTGCTTT
GTGGACAGTGATGATATTATTCCGAACTATGCACTTCAACTAATGTATGA
CGGTGCAATATCCACTGGTTCAAAATTGATTACAGGTGCAACGAAACGCT
TTAATTCAGAAGGTGAATGGTTCATTCCGATGCATATCCAATATAATATT
GCCAAGCCAGGAATGAAAACACTACTGAAAAATCCCGAGCTATTTTATTC
GATAGGTCCTTGTGCGAAACTCTATCATCATTCTTTGATAGATGGTGTGT
TTTTCCCTGAAAATATTCGTTACGGTGAAGATCAACCATTTGTTTTGCAT
GCTTTACTTCAAGCAGAAAATATCTATACTGTAGAAAAAGTAGTTTATTA
TTACCGCTTACGTGATGGTGAATCGCAATCATTAACGCAATCTGTAAATA
AAGATCCAATTCGTATTTTAAAATCTGTTTTTCAAATATTTGACTACGGG
GAAGCAGAGTTACTCAAAAATAATACGGAATATGAAATAGCTTTAAAATA
TTATCAACGAGTATCTAGTGTTGAGCTTTGGGGCGCTTTAAGGGCGGCTA
TTGAAAGTAAGAAAAGTGAAAACCAACAAATTGCTTTTACGATGACATTA
GACTGGTTAAAAACCAAATCAGATGACTTCCTAAATATAATTCCTTCCTT
TAGATATTTCTTGTTGTTCAGTAGTATCGAACGTGTTCGCTATATTACAA
GAGACAATAAAGAAAACTATCGCCAACTAATTACGTATTTGTGGGAGAGA
CAAGGGGAAGAAGCAAAAATTGCATTTAGAAAAGCATATCCTATCCATAT
GAAAGCTGCTTTACAAATTATGGAACATAATAATTGGGGAGCCGCTCGTA
AAATCTCCTTTAAATTTATTATCCGTCGTAAATTTAAGGCGCCTATTCTA
ATTCGGAAAATAAGCAGAGGCATTATATTTAGACTAGCTACTTTGATGCC
ACGCAAAAAAGACCAAGTTATTTTAGCTACAGAACGTAGTACAAGCTTAG
AAGGAAATTTATTAGCCATTTATGATTACCTGTTTTATAATGATATGCCA
CAAAAAGTCTATGTGTTTTTACGGAAAAATCGTAATTGGTTTGAAATGTT
CCAATTGTATTATGCATTAGGGCGAACTAAAACAATTGTATTAGATGATT
ATTACAACAAAATTTATGGTTTGAAATTTAATAAAAAGACACATGTGGTA
CAATCATGGCATGCGACAGGGGCTTTCAAAAAGTTTGGCTTTAGTGCGCT
TGAAGGTACAGATGCTAATACAGAAGAATTTGAGACACGTGCTCATTCGC
CTTACACAGATGTTCTTGTTAGTTCAGAAGGCATAATTCCTGAATATATG
GAAGCTTTTAGAAAACAAGCTAATCAAATTAAACCTATTGGTGTGCCTAG
AACAGATGTGTTTTTTGATCAAGAATATGTGGCATATACAAAAGAAAAAT
ATATGAAAATGTATCCGCAACTTCGCGACAAAAAAGTGTTGCTATATGCG
CCAACTTTCCGCGGTGGGCCAAATGAACGCTTTAATTATAGCGTTGTACT
TGATATTGCTGCTTTGAAAAAAGAACTTGGTGATACACATATTTTAATTT
TAAAATTCCATCCTGTTATTAAGAACGTTTCATTTAATGTGGATGAAAAT
GATCCATTTATTTTAGACTTGACGTTGAATAACGATATTAATGATTTAAT
GTTATTTAGTGATGCGCTTATTACAGATTATTCTTCGGTTATTTTTGAAT
TTAGTTTAATGAATAAGCCAATTTATTTCTTTGCATATGATATTGATGAT
TATTTGGATGAACGCGGATTTTATTTCGATTACAAAGCAACTATTCCTGG
TGAAGTTTTCAAAGATACGCCGTCGCTCATTGAGTCGATTAAAACGGGAA
AATATAATTACGACGAACTGGAAGTCTTTAAAAAGAAATTTGTTGGAAGC
TTAGATGGTAATTCAACGAAGCGTTTTGTAGAGACCTATATTGTTCAAGC
AAATGAGGAAGTGAAAGATTTATGA
ORF1445 (SEQ ID NO:17)
ATACCTCGTTTATTTCTAAATTTATTCACGTTTTTTTCATATTTGAACTC
TAAAATAAAAGGTATTATCTTATGGAAAGAAGTGAAGAATATGAAGAAAC
GGATACAACATTTAGATTTTTATTTTGTTGGAATTATTGTTATTGCCATC
TTTTTTAATTTTTATGGTATTTGGAATGATGATACGGTAAATCCTTATTA
TACAGCTGCTGTGACAAGTATGGTGCAAAATATTCATAATTTTTTCTACG
GAGCTTTTGATCCTGCTGGTTTTATTACGGTAGATAAGCCACCTGTTGCA
CTTTGGTTGCAAGCGATCAGTGCCCTTATTTTCGGCATACATGGTTGGAG
TGTTATCTTACCACAAGCGTTGGCTGGTGTTGGTTCGGTGATTTTGCTTT
ATGTGCTCGTCAAACCTAGATTTGGCGCATGGGCTGCAAGAATTACTGCT
TTGATTATGGCGCTGACTCCTATTGCGGTAGCCGTGACGCGTACTAATAA
TATGGATGCTATTCTTGTATTTGTGTTATTACTAGCAACCTTTTTCCTTT
TTAAAGCTGTTCATCGTGCAAAAATCGGCTGGCTTTTACTTTCTTTTGCG
TTGATTGGTGTCGGTTTTAATGTAAAAATGCTCCAAGCTTTCATGGTCGT
GCCCGCATTTTTGCTCTTCTATTTTATTGCTACAAAACTAAGTTGGAGAA
AAAAGCTAGTTCAATTAGTAATCGCACTTGTCTTAATGCTAGGTGTCTCT
GTTTCTTGGGCAGTCGTAGTTGATCAAACAGCTGCTTCAGAGCGACCTTA
TATCGGTAGTAGTCAAACCAATTCGGTGCTTGAGTTGGCATTTGGCTATA
ATGGTATGGAACGGCTTCTTGGTCAAGAAACTGGAACAGGTAGTAATGGT
AATGTGGAAATGGGGACTCCTCCTAGCACTAACTCAACAGATTCAAGTAA
CGCAACCCCACCGACTCCTCCAAGTGGAAGTATGCAGGACGGTGCTAACG
GCGGAACTCCTCCTCAAGGAAATAATGGCACTCCTCCAACTGGAGCTCCC
AGTAATGCGGCAAACGGCGGCGGGAATGGATCAAAAATGACTGGTAGCAC
AGGCATGTTTGGAACAGGGAACGCTGGACCACTTCGTCTATTCCAAACCG
CACTTGGTGATCAAATTAGTTGGTTCTTACCACTTGCTATTATCGGTATG
CTAGCCATTTTCCTGGTGTATCGAAATGAAAATAAACGAATTTATCAATT
GACCTCCAGACAAAAGGAAATAGTTTTCTGGGCAGCTTGGCTTATCCCTG
TCGCCGGATTCTTTAGTATCGCTGGCTTTTTCCATCATTATTATTTGATT
ATGCTAGCTCCGCCAATTGCTTTACTTAGTGGCGTAGGTCTAGTGGCGCT
CTTTCGGTTATACCAAGACAAAGAGAATTGGCAAAGGTTCCTGCTACCTG
CTGCAGTTACGCTAACTGGAGGTCTACAAGCATTCTTTGTTGCTGCTTAC
TTGCCTGTTTTAGCGGTAGTTATTGGTATTGCTGCACTAATTACGTCTAT
TATCTTAATTGTACTTAGACCACAACCTTCCAAACTCACCGCGAAAATAA
CAGCGCTCGCTTTAGCGATTTTGCTCATTGCTCCAACATATTGGTCTTTA
ACACCTATTTTATATGGTGGGAATAGTTCCTTACCCGAAGCTGGACCACA
ATTAAAACAATCTAGTGGTGGTGGATTCGCCGATGCATCTGTTGATAGTG
ATTTAATTAGCTATTTACAGAAAAACAATACCGGAGAAACCTACTTATTT
GGGACAACAGATGCAACAACAGCAGGTCCTTATATTATTAAAACGAAAGA
AGCTGTTATGGCACTTGGTGGTTTCAATGGAACAGATCCAACTCTAACTG
TAAAACAATTAAAACAAATGATTCAAGCCGGTGAAATAAAATATTTCTAT
CTCCCATCAAATAGTAAAGCTTCTGACTCAGATGTGGTAAAATGGATTCA
AGAAAACGGTACAGAAATTGATAGTTCGAAGTGGAGTAGCTCCGGTTCTA
CGGATGATGACACCACTTCTAGCGCTTCTTTTAATATGCAAGGTGGCGCT
CAAGGAATGAATGGCACTGGTACAGGAACACTATATCAATTAAAATAA
ORF149 (SEQ ID NO:18)
GCTTTTAAATTTTATGAAATGCCTTCTACGGATATTGCTTACTCAAGGAC
CGGGAAAATCACTAACACTTCTGGACATGCAGTATGGACTCAACCTTGCG
GACAAATAAACTCTACTTTAAAAGGACCAGCAAGTGATTATCTTAACAAA
GAAATAACTATTTGGAGAAAAGTAGAAAACAAAAGAGGTACTTATTATCA
ATTTAGTGAAACCAAAACTCCTAATATAAAAGCTTGGTTAGATGCAAGAG
CCATAACACTCTATGACCAAGTACATTTTAATGAAGAGTACAATCAGATG
GCAGTTATATCTACAGTTATTGGACATGCAGTATGGTCAACACCTTATCT
TCAGTCCGATAGTAAATTAATTGCGCCTGCTTCTAATTATGAAGGAAAAA
GAGTTGAAATAATCAGAAGAGCCAAAACAACAAGGTCAATATATTATCAA
TTTAGTTATGATAATAAAGTAATTGGTTGGTTAGACACTAGGGCTTTCTC
ATTAATACCTTCAAACACTGCTATGGTGATAAGTAATAGTACAAACGACA
TTTTTTCAAATATAACTGATGCATATAATAAAAAAACCCTATAA
ORF1490 (SEQ ID NO:19)
GGAGCTGGAATAATGACAAAAGAAGCAGAAATTGGAAAACAAATATTTAT
TCATGTCGGTGGAATGGAAAACGTTTCAAGAATTGCACATTGTATGACAC
GAGTTCGATTGGGGATAGTTGATAGTGACCTTGTTGATGTAGCTGGATTA
AAAAAAGTTCCGGGTGTTATCGGTGTTGTAGAAGATGATACTTTACAGAT
TATTGTTGGACCAGGAGTTGTGAACAAAGTGGCCGGCGCAATGGCAGAAA
TGGCTGGTGTTAAAATTGGCGAAACGATTCAAGAAAATTTGGATAGCGGC
ACAAAAACTGGTAAGGAATTAGTCGAAGAAAAAGCATCCAAAACAAAAGC
TGAATTAAAAGCAAAACAAAATAATTCATCTGGTTTTAAAAGATTACTAA
AATCAATCTCTAATATTTTTGTTCCACTAATTCCGGGTTTTGTTGGAGCG
GGATTAATTGCTGGTATTGCAGCAATTATTTCTAATAATATAACAGCTGG
AAATTTAGATGCTGCTGTTTGGACTCAATATATTGATATTTTAGGAGTTA
TTAATAAAGGAATATTTGCCTTCTTAGCTATTTATGTAGGGATTAATACC
GCGAATGAATTTGGTGGAACACCTGTACTCGGTGGTGGAATCGCTGGGAT
AACATTGCTTTCCGGATTAGCAGAAGGACATACAATTACAAACATTTTCA
CGGGGGATCCTATCGTAGCCGGACAGGGTGGCATTATCGGCGTATTATTA
GCAGTTTGGTTAATGTGCGTACTTGAAAAAAACTTGCGCAAAATCATTCC
GAATGCGATAGATATTATTTTTACGCCAACGCTTGTATTACTTATTATTG
GTTTAGTAACTATTTTCTTAATTATGCCTTTTGCAGGACTTGTATCAGAT
GGTTTAGTTAACGGTATTAATTGGGTAATCGAAGTTGGAGGAGTATTCGC
AGGTTTTGTGCTAGGGACGTTATTCTTACCAATGGTTATGTTTGGGTTAC
ATCAAGTTTTAACACCCATCCATGTAGAAATGATTGCTCAAAATGGTTAT
ACTATCTTACTTCCTATTCTAGCTATGGCAGGTGGCGGACAAGTTGGTGC
AGCAATCGCGCTTTGGATTCGTTGTCGTAAAAATAAACCACTTGTAAATA
TGATTAAAGGGGGTCTACCGGTTGGAATTTTAGGTATCGGTGAACCATTA
ATTTATGGGGTAACAATACCACTTGGGAAACCTTTTCTTACAGCTTGTCT
CGGAGGCGGTATCGGTGGGGCAGTAATCGGCTACTTTGGTAACGTCGGAG
CAATTGCAATCGGCCCATCAGGAGTTGCACTTATCCCACTAATCGCGAAT
AATGAATGGCTTTCTTACGTCATCGGATTAATTGCAGCATATTTAGGTGG
ATTCATTTTAACTTATTTCTTTGGTACACCAAAAGACGCAATGAATAGCG
TAGAATTGTAA
ORF1491 (SEQ ID NO:20)
ATAAAAATTTTAATATTAAAATTATATATTGAAATTTTATTTCATTTATA
TTATACTGTGTTGGAAGGAGTGGTTAAAATGTTAGAGAATTTAGCAACAG
AAGAACGAAATGAAAAAACGATTGACTTAGACACACTTTCTCCAAAAGAA
ATTTTAGCAGTAATGAATGAAGAAGATTTAACAGTTCCAATAGCTATAAA
AAAGGTCTTACCTCAAATCGAGTTAATTGTAAGCGGTGTCATCTCGTGCT
TTCAAAAAGGTGGCCGTTTGATTTATCTTGGTGCCGGAACAAGTGGAAGA
TTAGGAGTACTTGATGCAGCTGAATGTGTTCCAACTTTCGGGGTCTCAAA
GGAGCAGGTCATTGGCTTAATCGCAGGTGGTGAAAAAGCTTTTGTAGCTG
CAATAGAAGGTGCGGAAGATTCAAAAATACTTGGCGAAAACGACTTAAAG
CAAATTAAGTTAACTGCAAACGATTTTGTTATTGGTATTGCTGCAAGTGG
TAGAACGCCTTATGTAATTGGCGCACTTGATTACGCCAAATCTGTCGGGG
CTAAAACCGGAGCAATTTCTTGTAATGCTAATGCAAAAATTTCAGCGCAC
GCCGATATAGCCGTAGAAGTTGTGACTGGAGCGGAAATTTTAACAGGTTC
TACTAGGTTAAAAGCAGGAACAGCTCAGAAACTAGTATTAAATATGATTT
CAACAGCTTCAATGGTTGGAATTGGAAAAGTGTATAAAAATTTGATGGTG
GACGTTTTACCAACAAATAAAAAATTAGAAGAGCGCTCTAAACGAATTAT
TATGGAAGCTACAGAGGCTGATTACGAAACAGCAAATAAATTTTATGAAG
CAGCTGAAAAACATGTCAAAGTTGCCATTGTCATGATCTTAACCAATTCA
ACCAAAGAAATAGCGTTAGAAAAATTAAGTGAAGCAAAAGGGTTTGTTCG
AAACACCATTCAAAAATAA
ORF1492 (SEQ ID NO:21)
CTAATTTTTTGGTATAATTACGGAAAATGGAATGGAGAAAAGTTAATGAG
TAATGGTAGTATTATCAATCGAATAGAAGGTATTTTAAATGATTTGCCGA
AGTCAGAGAAAAAAATTGGCCAAGCAGTATTAGCAAATCCAGAATTTACA
ACTACTGCATCGATTCATAAACTAGCTCAAAAAGCAGATGCAAGCGGTGC
GGCCGTTATCCGTTTCTGTAAATCAATCGGACTTCAAAGTTTTCCAGAAC
TAAAAAGACAATTATCATTAGATTTGGCTCAACCGCAAAAGAAAGGCTAT
TATGATATTGAACCAAATGAAGATTTTCATACCATCACAGAAAAACTAGT
TTCCAACATGATTCAAACGATGAACGATACGGCTAGCCAACTAGATGAAG
CCAAAGTTTTAGAGGCATGCGAATTACTCGGCGAAGCAGATACTATTTAT
ACATACGGGGTCGGGGCTTCTTGGTTAGTAGCAGAAGATATTTCACAAAA
GTGGCTGCGTGCAGGGAAACATGTTTTAGCTACGCAAGATGCACATGTTC
TAGCAATGGCTTTTGCAACGGGGAAGAAGAAAGCCGTTTTTATTGCTATT
TCTAACAGTGGAGAAACATCAGAAGTCTTGCAACTCGTTGATCAAGCCAA
ACTGAACAACGTCATTGTTATTAGTCTTACACGTTTTGGTAGCAATAAGC
TGAAGGAAAAAGCAGATTTATCTTTAGAAACTTCTCGTGCTCCGGAAGCT
GAAATTAGAAGTACAGCAACAAGTTCAAGACAAGCACAATTGCTAGTAAT
TGACATTTTATTTTACTATTATGCATCCCATCACTACGACGAAATGATTC
AACAAATTAAACATTCTCGAGAGGCCACGAATCGTTTTAGAGAATAA
ORF150 (SEQ ID NO:22)
TTTTGTAATATAATACATATAAATGAAAGAGGGAACAAAATCATGTTAAA
GAGAAATGTACAAAAAGGGATGATTAGTTTAATTGCAATTATGATGTTTT
TATCAATGTTCAGTTTTACAAATTTAAATTCTATTAAAACTGTAGAGGCT
GCAACAACTGCGTACAATACAATTGAGTACAATAAATCAGTTAATTTAGA
TGCTAATATTGCATATCCAGAAACAAATGCATTTTGGTCTGCACCATATT
ATTCAGAAGGATCAACATTTATATCAAGTGCAACTGCACCTTCTTATGCA
AAAAAAGATGTTAAATTAATAAGAGAGGCAAAAACTGAAAGAGGAATTTA
CTATCAAGTAAAATTGGGAGACAAAATTATTGGTTGGCTTGATAAATGA
ORF1504 (SEQ ID NO:23)
CGAATAAATAATTACGAATACATTTCAAAAAACAAGGAGGAAATTAAACA
TATGCAAAAAGCAATCAAAATAATGTTAGTTTTATTTTTAATGACAACCG
TATTTTTACCTTTTAGTAATGTTCGGGCAGCATCAACTGATGTAGTAAAT
ATACCAGATCCGTATTTAAACGAAGGTCTTAAGAGCATCGTAGGAAATCC
ATTCTTAACCGAATTGACTGAAGCGAATCTTGAAACCATTAGTGTCGCTG
ATATCTCTTATATGAATGGAGTACCTGGTTATGCTGTCACTGGTTTAATT
AGTGATTTAACCGGACTCGAAAAAGCAGTTAATATGACCAAATTATATTT
TTCAAACCAAACAGAAATTAAAAATTTAAATCAAATAAAAGATTTACCTA
ATCTGAAGAAAATTGTTGGTGTTACTACAGGTTTAAATGATATTAAAGCC
CTTGGTGAAATGCCCGCACTCGAAGAACTAGAATTAGGTGGGGATTATAT
TACTGATTTCACTCCCTTACTTGAAAAAGATAATTTAAAATCATTTTCTT
ATAACTCGTATGCTTGGTTAAATCCAGCATATCACCAAATTGATAATGAA
GAATTTAAAAAATTTACAAACCTTAAATCACTCGAAAGTTTAGATGTAAC
TTGGAATAACATTACCGACTTATCATCATTAACTGCAAATGATCATATTA
CAAACTTAAATCTAAGTTACAATAAGTTCACTAATATAGCTCCAATTGCT
ACAATGAAAGAACTTAAAGTCCTTTATTTAAACAATAACAACCTAACTTC
CATTGATTCTTTAAATACACTAAGAGGATTAACTATAGCCTATGCTGATA
ATAATAACATTACTGATTTGAGTAATTTAAAAGACTTTTTTGAAGGCATG
GATGTCGTTGGAGATTATAAAGGTTTACAAGTTAACAACCAAACTATCAC
ACTTCCAACTATTAATATCAAAGAAGGTGGAACAGCTATTTCAAATAACC
CTACTTTAGATATTGATGGCGAAAAAATGCCTGTTTCTAGCATTTCTGAT
GGCGGAACAGTATCTACCGATAATAAAACCGTTTCTTTTACTAACTTACC
AGTTGGAAATAAGACTGTTACTTATAAAGCAACATTCACAGCTACCTCCA
CTAAAGGAGTACCACTTAGTTATTCTATTAAAGTATCGCAACCAATTAAT
GTATCTGCACAATCCGATTCAACAGTAAACGTATTTTACAAAGATGAAAA
TGGCGATGAATTAGCTCCAAGTGAAACAATTTCTGGTAAATCCGGAGAAA
ATTATCAAACAATAGAAAAAACAATTACTAACTACACATTAAAAGAAATT
GAAGGCCAGCCTTCAGGACAATTTGGCGATAGTGATGCAACAGTCACTTA
CGTTTACGAAAAAGCAGACGGCGCTCCTGTTACCGTGAAATATGTCGACG
TGGATGGCAACGAATTAGCTACTTCCGACACACTGAACGGCAAAATTGAT
GCTCCTTATCAATCTACAGCCAAAAGCATCACTGGTTGGACGGTCAAAAC
GACTCCTGCCAATGCAACGGGCGTATTCACAAACGCTAACCAAACGGTCA
CTTATGTGTATGAAAAAGCGGATGGCGCTCCTGTTACCGTGAAATATGTC
GACGCGGATGGCAACGAATTAGCTACTTCCGACACACTGAACGGCAAAAT
TGATGCTCCTTATCAAACTACCGCTAAAAGTCTTTCTGGTTGGGCAGTCA
AAACGACTCCAACCAACGCAACGGGCGTATTCACAAACGCTAACCAAACG
GTCACTTATGTGTACGAAAAAGCGGACGGCGCTCCTGTTACCGTGAAATA
TGTCGACGTGGATGGCAACGAATTAGCTACTTCCGACACACTGAACGGCA
AAATTGATGCTCCTTATCAATCTACAGCCAAAAGCATCACTGGTTGGACG
GTCAAAACGACTCCTGCCAATGCAACGGGCGTATTCACAAACGCTAACCA
AACGGTCACTTATGTGTACGAAAAAGCGGACGGCGCTCCTGTTACCGTGA
AATATGTCGACGCGGATGGCAACGAATTAGCTACTTCCGACACACTGAAC
GGCAAAATTGATGCTCCTTATCAATCTACAGCCAAAAGCATCACTGGTTG
GGCAGTCAAAACGACTCCTGCCAATGCAACGGGCGTATTCACAAACGCTA
ACCAAACGGTCACTTATGTGTACGAAAAAGCGGACGGCGCTCCTGTTACC
GTGAAATATGTCGACGTGGATGGCAACGAATTAGCTACTCCAGATACATT
GAACGGTAAGCTAGATACATCTTACGCGGCAACAGCTAAAAACTTGAGTG
GTTGGAAGCTAACAGCCACACCAGCCAACGCTAATGGCGTATTTACAACT
GATGCTCAAACAGTCACCTTTGTATATGCTAAACAAGAAGACAATCCTAA
AAAAGAGGATAAAAACAAAACACCTATTAAAATCAGCGAAAACAAACCAA
CAGCAAGCAAAGTAACTAGAATCAAAAAACAAACAAAATTACCGAAAACT
GGTGATAATCAACAAGATAGTATATTGTTTGGATTAATTGGTACATGTTT
CGTTCTCTTAGGAATTTACTCTATCTCTAAGAAAAACAGCTAA
ORF1590 (SEQ ID NO:24)
TATTATAAAGGAGATAAGTTAATGAAAAGAAAAAAAACAGCATTACGTAT
CTTGGTAACTCTGGCTGTAGTAATGGCAATTAGCTTTTGGGTAGGGACTA
GCTCAAAAGAGGAAGTACAGGCGGCAGTAATTGATCAACCGACGCCAATT
AATGAAATTTTTACAGATGAGAATTTAGCAAACGCAATAAAAGCAACTTT
AAATAAACCTAGTACTACATCTGACGTTTCACAAGCAGAGCTAGATTCTA
TAAGTGAAGTAACGGCAGAAAGTAGCAATATAGCTTCTTTAGAGGGGGCG
CAGTATTTAAATAATATAGATACATTAATTTTAAATAATAATAAAATAAC
GGATTTAAATTCACTTGCAGGACTATCTAAATTAAGGATATTAGAAGCAA
ATGACAATCAATTAAGCGATATAAGTGCACTAGCAAATGTGACAAGTCTG
CACCAATTAAGATTAGCTGGAAATCAAATAAAACAGTTAAATGGAGTTAG
TAATTTAATCAATCTAGAAACCATAGAACTAAGCAACAATCAAATAACGG
ATATAAGCCCAGTTTCAGGTTTGAAAAACTTAATTGGTCTTGGAATAGAT
TATAATGATATCAGTGATTTAAGCCCAATTGCTGGCTTAGCAAAGTTAAG
TCATTTAACTGCAGACAATAACCAAATAAGTGATTTGAGTCCTATTTCAA
GCTTAGGGGCTATGGAAATTATGCGACTAGATAAAAATCAAATCAGTGAT
GTAACCCCAATTGCGAATTTGGCTAATTTAAGCTATGTATTTTTAGCTGA
AAATCAAATCAGTGATATTAGTTCTTTACAACCACTTTTTAATTCACCTA
ATTTTTTTGGTATTACTTTAGACAATCAAAAAATAACGAGTGAACCAGTA
TTGTACCAACAAGAGCTTGTGGTCCCTAATAACATTAAGGATGAAATGGG
AGCGTTAATCTCACCAGATACAATTAGCGATAATGGTGTTTATGCAAGCC
CGAATATTAAGTGGAACTTGGCTAATTATACTAACCAAGTAAGTTACACA
TTTAATAAACAATTAGCACAGGGTTCTTTTAGCGGAACAGTGACCCAACC
ACTTCACAATGCTTATACAGCGACATTTGACGTAGATGGAGTGAAAACAA
ATGAAGTAGTGGAAGAAACGAAATTGCTTCAAGAACCAACAGCTCCAACG
AAAGAAGGATACACTTTCACAGGCTGGTATGATGCGAAAACTGGCGGGAA
TAAATGGGATTTTGCGACAGACAAAATGCCAGCAGAAGACATCACATTAT
ACGCGCAGTTCACGATTAATAGCTATACAGCCACATTCGACATCGATGGG
AAATTAACGACGCAAAAAGTCACTTATCAAAGCTTGCTAGAAGAACCAGC
AGCGCCAACGAAGGATGGCTATACGTTCAAAGGTTGGTATGATGCTAAAA
CAGGTGGAACTAAATGGGATTTTGCGACAGGAAAAATGCCGGCGGGAAAT
ATAACGCTATACGCCCAGTTTACTAAAAATGCCACCCCAGATTCCAATGA
CCCAACAACGGTTACCCCAACAGGAAATGGCAATGGGACAAGTACCCCAA
GCAATTCAGGTGGAAATACCACGCTTCCGACAGCTGGGGACGAAAATACC
ATGCTTCCAATTTTTGTAGGAGTTTTCTTGTTAGGAACAGCAACGCTTAT
TCTCCGCAAAACAATCAAAGTAAAATAA
ORF1654 (SEQ ID NO:25)
GTGCATGTAGTACCTATCGCGAATGCGTGGAATTATTACAAACTATATAT
AATAACAACGAGTAAGTTTGAAGGAGTGAGAGGTATGAAAAAACAAATTA
TTACTATTGCTGGCGGAGGAAGTACTTATACACCGGGAATCATTCAAGCT
GTACTTAACGGGCAAGAACGATTACCAGTAAGTGAAATCCGTTTGTATGA
TATTGAAGCAACAAGAAATGAAAATATGTATTTAATTCTGGATTATATGC
TAGAACAACAAGGCTTTTCATCAATTAAATTAGTACAAACAACCAATCCT
GAAGAAGCATTTACTGGGGCAGACTTTATCTTTTCGCAAATTCGCGTTGG
CGGACTTAAAATGCGCGATAAAGATGAAAAAATCCCACTAAAGCATGGAT
TAGTCGGACAAGAAACATGTGGACTTGGCGGATTTTCTTACGGTTTACGT
AGTATTGGCCCACTGTTAGAATTAGTTGGCTTTATTCAAAAATATGCGCC
AGAAGCTTGGATTTTAAATTATACGAACCCAGAAACGATTATTTCTGAAG
CTGTACGAAGACAATTTCCTGGTATTCGAATGATTAATGTATGTGATATG
ACAATCGGAATTGAAGATACACTAGCTAAAAATTATGGCTATGACCGTGA
TAATTGGATTGCTACTTACTATGGTTTAAATCACTTTGGTTGGTATACGA
AAATTTATGATAAGTCATTAAAACGTGATATTATGCCAGAGCTTTTAGAG
AAGTTGAAAGTCCAAGAAATCAAAGAAGAAGACCCAAGTTGGAATCGCGC
ATTCAATATGATACGCTTTATGGTTCAAAATTTTCCGGATAACTTGCCAA
ACAACTATTTAGAGTATTATTTGTATCCAGATATGTATGTCGATTACGCT
GATCCAAATTATACCCGCGCGAATACAGTAATGGATGGTCGTGAGCAAAA
AACACAAGAAATGGCAGATAAAATCCGCAACAAAGAAAAAGGAGATGTGC
TTGATTTCTACTTTGGTGTACACGGCTTATATATTGTAGACATAGCTACT
TCGCTACTAAATGATGAGAAAAGTCGCTTTATGTTAATCGTTGAAAACAA
AGGATCCATCCCGAATTTACGTAGTGATGCAGTTGTTGAAGTTCCAGCAT
ACGTTGGTGCGACAGGTGTAGAAGCAATTGCCTTGCCTGCAATTGGTGAT
TTCCATAAAGGTATGATGGAAGCGCAAGTTGCTGCGGAGAAACTACTCGT
AGATGCGTACTTTGAAAAATCTTATCAAAAAGCATTACAAGCATTTACAT
TAAATCAATCGGTTCCAAATGCCACAGTGGCGAAGAAAGTATTGGATGAA
ATGATGATTGAAAATAAAGCATTTTGGCCAGAATTAAAATAA
ORF1655 (SEQ ID NO:26)
TTAAAAATGCTAAATCAACGTCAAAAAAACATTTTAAGCACACTGTATAA
TGAAAATAATTGGCTACTTGGGAAAAAATTAGCAGATTTATTCCAAATAA
GTGATCGAACTATTAGAAATGATATACGTGTTATTAAAGAGAGCATAGGC
GACGATTTTATCTTTACTTCTAAAAAGTTAGGATACGCCTATAATATGGA
AAAACCTTTTCCGATTGACGTTGAGGCAGAAACTGGATTTGAACAAAATC
GGATGGCGCAATTAATTCAGCAATTACTTGTCGAAGAAGGCGTAGATATT
TACGAATATGGTGCCGAAACATTCACTTCAGAATCAACCATTCAACGCGA
TATTCAGTGGTTACGAGGCTACTTTGAACAACTTTTAGGTTTAGATGTAG
TAATTCATTCTAGTGATGGTGTATATGCCATAAGCGCTTCCCCCACAACG
AAAATGGAGCTATTAAATCGCATTGCAACGTTGGATGAAGGATTAAAGAC
CAATTTATTAACAAATTGTTTTCCAGAAATTAATCATGAAAAAATTCGCC
AAATATTATTAGAAATGATTCATCAACATAAAATTGTTTTAAAGTATTTT
GATGAAACTATTTTGCTTGCACAACTTATTTATGGAAGTGCCTTTTTTCA
AAAACATGCGGAACAACAAACTTCTACGAAAATTGCTAATCCATTTCTTA
AACAACTTTTTGATACAATTCAACAAGAAATGGGTTACCAGATTTCGGCG
GAGATGAAGCAATTTATAGTATCAGAATATGAAAAAATTGTTGCAATGAA
TCATTTTGAAAACCAAGTAACGACAAAAATGGTTTTAGAAGGCGAACTTT
ATCAAGAAATATTATTGATTTTGGAAGAAATTAAGCATGTATATTTGATT
GATTTTACGGCAGATTTAGACGTTACAAGTGATATGACGAAACATATTTT
TATTGCGCTGGAACGTGCCAAACGCGGAATTGTCATTAAAAATCAAGTGA
CGCATATCATTACGCAGCAGTATAGCTACTTACTGGATATCGCGATTTTC
ATTGGAGAAAAATTACTTGAACGTCTAGGTGTAATTTTAAATCAAGAAGA
AATTATTTTATTGGTTATGTATTTATACCAATACTACCGAAAAATCGAAG
CGAAGCACCAATTAAATCAAGTAGTGCGCATAGCTTTAATTGTTTTAGAA
GGTAAGGCTGCGATGTATTACTTGCGTGAACAATTAGCCGAAGTATTACG
ACCAGTAAACGCTGAAGTTATAGAAATAACCGATAATTCCCAGTGCCAAT
TACTGTTAGCTGAAAATATCGATGTTGATTTATGTATTTCTACTAAAAAA
ATTGATTTGCCTGCGGAAGTTCCGTGTATTATTTTGGCTAATAACATCGG
TTTGATTGAAGAAGTAACAATAAAAAAACAACTTTCAGCCACAGTAGAAG
CAAATAAAATGAAAAAATTTGCTTATATTAAAGAAAAATATTTACACGAA
GAGCTTTTCTTAACGGATTATGCGTATGAGCAAAAATATAATGCGATCGA
ATTTTTAAGTCAATATTGCATTGATAAAGAATATGTTCCAGAGCGGTTTA
CTGAGAAATTATATAATCGTGAACAACTATTTTCAACAGCGATTCCGACT
GGGATTGCCTTACCCCATCCAATTAAAAATGTGGCGCGCAAAAGTGGTAT
TTTCATTTGCATTTTGAAAAAACCGTGTGCTTGGGATACACATAAAGTTA
GTTTGATTATGATTCCAATGATTGAAGAATTGGACGGGACAGAGGCACCT
TTAATTAATGATTTTTTATCACTAATAGCTTCTAATAAGTCCTATGTAGA
GCAAATAAGTGCATGTAGTACCTATCGCGAATGCGTGGAATTATTACAAA
CTATATATAATAACAACGAGTAA
ORF1680 (SEQ ID NO:27)
ATGGAACAAATTATGACTCTTTTACAGAGTATAAAGTTAGCGCTAAAACA
ATTATTGTCCACCAAATTTCGAACATTTTTAACAATGCTTGGAATTATTA
TTGGTGTTTTTTCTGTTATATTACTTGTTTCGATTGGGGAAGCTATATCG
AAAAATGTCTCCACACAATTAGGTGATATGGGAAGTAATCTTTTGACTGT
TAATTTTTATTCAGATAATCCAAACGATAAATTCACATACAAAGATGCAA
AAAAACTTTTGAATGAAGATGAAATTGGTTCGCCGGAAGTGATGCAGACA
AAGGAAGTTAGAACAAGTGAAAAGACAGCTAGTAATCAAGTAGCGGGAAT
AAATGAATATTATGCGAGCATAAAAAATTTGGAATTAGCATCAGGCCGTT
TTTGCTCGAATGTAGATGTTATCTATGCCCAAAAAGTGACAATTATTGGC
TCAGAAATTGCAAAAACTTATTTTAAAGAACAGAATCCGATAGGTGAATA
TTTGCAAATTGCAGGTGCTAGGTACATGGTCATTGGAGTTTTAAAGGAAA
AGGGGGAGAGTTTATTTGGATCGGCAGACAAAAAACTTTTTATCCCAATT
TCTTCCGCAGAACGACTTTTTAAAACAGATACTGTGGATTTGTATTATAT
TCAAACAAAAAAAGTAGAACAAGTTCCAGCAGCAGTTAAGGTAGTAAAGA
AAAAAATGCGCCAGTTTTTTCCGAATAATGAAGATGCGTATACGGTTGTA
AACCAGCAACAGGCGTTAGATACGTTTGATAGTATTACGGGAACACTCAC
GATTGGACTAGGTGCGATTGCAGGGATTTCGCTACTCGTTGGCGGTATTG
GTATTATGAATATTATGCTCGTGTCTGTCTCAGAACGAACCCGAGAAATT
GGAATTAGAAAAGCGATTGGCGCAAGTAGTGGCAATATTTTAATGCAGTT
TTTGATTGAAGCAGTTGTGCTTAGTTTAGTAGGCGGTTGTATCGGGATTT
TACTAGGAATTTTTTCCGCGCAAATAGTTACAACTACATCAAGTTTTGAA
ATGTATGTTTCTGCTTCTACGATTCTTTTAGCGGTTGGTTTTTCGATGTG
TATTGGGATTGTATTTGGTGTAATTCCCGCACAGAAAGCTTCAAAAAAAA
AGCCGATAGATGCGTTACGGGCAGATTAA
ORF1681 (SEQ ID NO:28)
TACGAAATCTATTCAAGTAGTAAAAGGTCTTACAGAGAAACAACTTGTGA
TTATTCCATAGACGGAGGTGAGCAAGACTTGATACAACTATTTAATATTT
CCAAATCCTATCAAATGGGTGAATGCACCATAAAGGCTCTAGATAATGTA
TCATTGCAAATTGATCAAGGGGAATTTCTGGCCATTATCGGACCGTCTGG
CTCTGGAAAATCTACTTTAATGAACATCCTCGGTATACTTGACAGAGCAA
CGATAGGAGAATACTACCTCAATAAAACGAATTTAATGCGAATATCAGAT
AAGAAAATTTCTCGAATAAGGAATAGGAAAATTGGTTTTATTTTTCAACA
ATTTAATTTAATGCCACGACTTACTGCTTTTGAAAATGTAGAATTGCCAT
TAGTATATCGAGGTGTGGGCAAAGCTACTCGAAAAAAAGTAGTATTAAAA
AGTTTAGAACGTGTCGGACTATTAGATAAAGAAAAACATATACCAGCCCA
GCTTTCAGGAGGACAGCAGCAACGGATTGCAATAGCTCGAGCCATCGCTG
GAAGTCCAGAATTAATATTAGCAGATGAACCTACTGGCGCGCTAGACTCT
AAAACTGGAGAAGAAGTAATGACTTTGCTAAGAGAAATTCATAGAGAAGG
AAATACATTAATCATGATTACCCACGACAAAGAAATTGCAAATCAAGCAG
AACGAATAATAGAAATAAAAGACGGAAAACTCCGTGAATGGAACAAATTA
TGA
ORF1683 (SEQ ID NO:29)
ATATTGAAAAAAATAAGCTGGGTGATTATCATGCTGGTTATCGTTGCTGC
ATCGGTTGGCTATTATTTTATAAAAGAAAATGAGCGAAAGACGCCACAAA
CAATTGATTACAAAACAGTTGAAACAAAAAGAACAGATTTAAGTGTATAT
GTTTCCGCAGAAGGTCACATTGTAAAAAAAGTAAATGAATGGCCAGATTA
TGAAGATTTTGCTGTCAAAATTATGGTAGATGAGCTAGAAATTAACCAAA
TAAAAGAAAAACAGACAGCTGATGTGTATGTCGAAGCAGTAAATAATAAA
GTATACAAAGGAAAAGTTGTAGATATAAATGAGAAGGGCATTATAAACGG
GTCAGTAACTTCTTATGCCGTGACGATTGATTTAGAGGATGAGACGAAAG
TAAAAGAAAATATGTCTGTAAGTGCTGATGTACTTGTGGCTCTAGAAAAA
AAGATTTTAACAATTCCAATAGAAGCTGTTAATACGGATAAAGCTGATAA
ACATTATGTCTATACAGTTGATGCAAATAAACAAAAGAAGAAAATTTGGA
TTGAAGTAGGCAAACATAATACGAAATCTATTCAAGTAGTAAAAGGTCTT
ACAGAGAAACAACTTGTGATTATTCCATAG
ORF1684 (SEQ ID NO:30)
ATGGTAAAATGGCATTCTAAAGCTGTTTTTAAACAGCTTTATAGAGAGAG
AGGGGAATCATTTATGCCAAAAATTGCTAAAGGTAATCGACTCGAGAATG
TTGCTTTTGAGTACATTAAGAACAAAATCACAACTGGGGAGTATCCAACG
GGTTATCGTGTTGTAGAAGCAAAGTTATCTCAAGAACTAAATATGAGTAG
GACACCAATTAGGCGAGCGATTATCAACCTTTGCCATTCTGGATTTTTAG
TCCACCAATATAATCGGGGTGCTTTTGTTCAAAACACTGAAGTAACTATT
ACAGAATTTTTTTCGCGAATGAAATTAGTCGAACTATTGATGTATGAAAG
TACAGAAAAATTAATTCTACGTGAAGATTATATTGTAGTGGACGATATAA
TAGAAATAGCAGAGAAAGTAATTCAATATGAAAAAAACAAAGAATATGAA
TTAATGCGCGATACGTTTGAAGATTTTATCGTCGCATTTATAGGTAAGCT
AAATAATGATTATTTTAATAGAATAATTCAAGAATTATGGAATGGAATCA
ATGATAATGCAACAAAAGAAGTAAGACTAATTATTGTTTCGGCAAGTGAT
CGAATTGCGGAAGAATTAGCTAATATAATTGAAATTTTAAAATCATGGAA
TTACGGGGAACTAAAAAAATGTTTTCAACACATAATGAACGCAATGATTT
TGATTGCTTTTTAA
ORF1717 (SEQ ID NO:31)
AAATTAACTACGAAAGAAGGAGGGGAGCTGATGAAGAAGTTTTCCATGCG
AGTGGTGCTTATTATTTCTGTGCTTTTTATTGCACTGGGGAACGCTAATG
TTTCTATCGCACAAGAAAGGGACACAACGAATAAGCTTCCAGAAGAGGAG
CTGGGTTCTCTGGATACCTCTAATCTTATTGCAGAAGAAGTCGCACAAGA
CAAACCAGCAGAGGTCGAGAACTTAGAAGAAATTCCAACTACGGATGAGT
TGATGCAAAATCCAGATGTTCGAGAGCAGTCTGTTGCCGATTCGGATGAT
CCGGATTTAACAGCGGTAAGCTCGGGTGATTACTGGACGATTTATTATAA
TTCTGCGAACGATGAATATAGCTTGCGTATGTTTGGCAACGTACCAAGCA
GTAAACCGAGCGCTTGGAATAGTTATTTGAAATACATTAAACATATAGAG
ATTGAAGAAGCTACTTTAACAGGGGATTTTTCATCGTATTTTAGAAGTAA
TGTTTTCACAGTACTTGAGAGTGTGAGGATTGAACGTTCAAATTTGTCTG
GAGTGACGTCTTTTGCAAGGGCATTTGAAGGTGACAGTGAATCAGAATCA
CCACTTGAAAAAGTGATTATCAGAGATAATTATTATCCGGAGACGCCTTC
TTTAACAAATATAAGTAGAATGTTTACCTTATGTCGTAAACTTAGTGAGC
TTGATGTGAGTGGACTCAATACAAGTTCCGTCACTAAAATGGATACCATA
TTTAGTAACGCGAATAGCCTTAAAGAACTGGATGTAAGTCATTTTGATAC
GAGTTCAGTGACTGATATGAGCTCCATGTTTGCTGCTTGTAATAGTCTTG
AAGAGCTTGATGTAAGTAATTTTGATACGAGTTCCGTTACTAATATGAAG
TATATGTTGAGTGGACTCCACTTGAAAAAACTAGATGTAAGCAACTTTGA
TACAAGTTCCGTTACCAATATGTATGGCATGTTTGCTTATTGTTACAATC
TTGAAGAGTTAGATGTAAGCAACTTTGATACAAGTTCCGTGAATAATATG
CTGCATATGTTTTATGTTTGTAATAATCTTGAAGAGCTAGATTTAAGTAA
CTTTGACACCAGTTCCGTTACTAATATGTTTGCCATGTTTGCTTATTGTA
CTAGTCTTAAAGAAATAGATGTAAGTAATTTTGATACGAGTTCTGTAACT
ACTATGAGTGCCATGTTTTTTGAGTGCAGCTCACTTGAAGCGCTAGATTT
AAGTAACTTTGATACTAGTTCCGTGACTACTATGGCTTCCATGTTTGAAA
ATTCTACAGCGTTAAAATCTTTATATTTAGATAATTTTACAGACGCAGCA
AGCATGACAGATATGTTTAAGGGAACCACCTCTTTAACCTACTTATTTGT
CAGTCACAATTTAAGTACTTTTAATAGGTTAGAAAATACAAGCTGGTATG
ATGAAAAGAATTGGGTGCAGTTTTCGAATCTCTCGCAACTACAGACATAT
CACCGGAAACAAAGTGAACCTACTGGCTATAGAAAAGGCGCTTTCCTTTC
TCTAACAATGGATGCGATGGGTGGAGAATTTGAAGATGCGGAAGAACAAA
AAGTACAAAATAAAGTTTCTGGGGAATACTGGGAAGAGATAGTTCCTGTA
AAAGAAGATTATTACTTTGACGGGTGGTATCTTGATCAAAACTTCACTAA
TAAGTTTGATTTTTCATTGCCTGCTGCTGTATCTACAACTATCTACGCCA
AATGGGTAGAAAACTACACGGTGATTATTCCAGCTTCTATTTCCTTAAAT
GAAGCTACTGAATTGAAGGTAGAAGGGATTAATCGAGGGAGTAAAGCTTT
GTCCGTGGGATTAAACCGATTAGCAACGTCTGTTTCTGAGAGCAACGAGC
TAACTTTAGCTAATACGGCAGACACGACAGTCCAATGTTCGGCACCGCTG
AGTTGGGACGGTTCCGAAAACAATCCAGAAAAAGCGATTTTAACCCTAGC
TCCAGGTTCGGAAATAACAGAAGGCGATGCAGTCATGGCTATTGAAGCGC
CAGAAAATATCCAGGCTGGAAAATATACAGGGAATCTAGTATTTTCGATT
AATTATGAATAA
ORF1718 (SEQ ID NO:32)
CATTCAAAGGAAGTGTTTTTAATGAGAGAATATTTAGATTCAAAAAGCCA
AAAAAAGGTAGCGCTTCTTGAGAAGATTTTTTATGCGGAAAATCATACAA
GTACACAAGAGGAACTATTAAATGATTTGAACATTACGTATCCAACCCTC
ATTTCTACGATTAAAACAATTAATTTTGATATTGAACGATTTGGATACAA
AGCGTTTTCGATTGTTCACTCCGCGCCAAATTTGTCCTATACCTTAAAAA
TATCGGATAATTGTAGTATTCAACTCATTATCAACGCCTATATTAGAGAA
TCGCCCAAATTTCAAATTCTCGAAACTTTATTGCTGGCTTCCTTCCCTAA
TCTACAAGCATTGGCAAAAAAAGTACATGTTTCTTATTCTGGGATAAAGA
AAGAAATAAAAGAATTGAATGAAGAGCTAAGTGAACGGAACTTGTATATT
TCTACAGGAAATCAGGTAGAAATAACGGGGGATGAATTTTCATTAAGAAT
ATTCTATGCCTTCCTTTTTTTAGTCGCATATAGCGGGGATCGTTGGCCAT
TTTCTTTTGTTCGATATGATGAAATCACTGACTTATTAGAGAGTTGTCCA
AAAGAAATTTATAGAGCAAACTCGATAGATAAGGCGATGATGATTCATTA
TTATGTCGCTATGCATTTATTAAGAGATCGAATGAATTGTCAAATTGATA
CAACTAGGCAGTTTAAAGTGGCCCTATATAAGGCTTGCACCGAGGAGTCT
AAGAAATCAGAAAGTGCGTTCATTAAAAAGGTAGCGAAACAACTTCCTAA
TAGGAACTATAAAGAAATGACCTACACGACGCAAATTATACTGAGTACCA
TTGTTGCTTTCGGCAGTTATTCTTCCATTGAAAAAATGCCTTCCTTTTTC
TACATGGATGAACAGTTAGAAGAAATGGGCTTCATGAAACTAGTTGATTT
TGCCAGTGAACGAGTAAATGATAATCTCTCTATTCCATTTTCAGAGAAAG
AGATGGAATTGCTCCGTTACTCCTTTGCGAGTATAAATTATAGATATTTC
TTACTTGATAATTTAATTAATAAGTTCAATAATATTGTGCCAGGGTATAC
AGATCTTGATCGAAATATAAGAAAAATCCATAAAGTGAATCACCTAGAAC
CATTGATTAGTCAATTAGTAAATTTAAAAGAAATGGATCCATTAAAACCG
TTTGAAGAAAGACTTACTTCAGATTATCTGATCATTTTGGATAAACGGAT
TGATTTTTCTATACACACCTTGCCTATTAAAGTAACTATTTTATCGACCA
TATCTAATGAAACAGCCGTATTTGATTTTATGAGGTATTTTTCTAGTTAC
TACAATCTGGAAATAATTAATCAAGTTGATCCGGTGGTGGATTTATATAT
AAGTGATTTTTCCGTATCTCCCGAAGTATTGACCTCTTTACGTATCAATC
AGCCAATAATCTATGTAAATACTAGATGGCTAGAATCTGATTATGTCAAA
ATTAATGATAATCTGGCGAAAATAGCGAGAAAAAATTTATCGCTAATAAA
AAAGATTAGGGAAACACTTGTTGGAATGGATATGGAAAATGTTTAA
ORF1739 (SEQ ID NO:33)
GAAGCAAGAAAATCGAATGTAAGTATTCGATGGTCATTTGCTAAATCCTT
AACTTCCCAGCTTTTATATGGAGAGGGAAAAAGAATGAAATTTGCTATTA
TAATGCCTTTTTACAATGCCGAAAAAAGGTTAGCACTATCCATAGATAGT
ATAATTAAGCAGTCTTACAGCTTTTTGAAGCATGTGGAAGTACTGCTTAT
TAATGATGGAAGCACGGATGGAAGTGGCGCTATCGCAAATCGTTACGCTA
CAAAATATCCTAACAATATTCGCGTGTTGACCGTTCCAAATGGTGGACCG
GCAAAAGCGCGTAATATCGGAATACATAATGTAAGTGAAGATACTGATTT
TGTTGGTTTTTTAGACGCCGATGATATAATGTCAGAGAATATGTTAGCGA
GTATAGTTACATTTCTAAATGAGTCTAACGTTTCTATGCTTGTGCCAGCT
TTTTATTACTTAGATGATTTTGGAAGTAAGAAAAAAATTTCACCACATAA
ATTAAATTACCGTTTTGCGAATGGAAATCGAGTTGCTGATATTGAAAAAG
AACCGGAAGCAATTCATTTTTATATTGGCGGTACTTTTTTACGTTATGAT
TGTTTGAAAGAATTTACTTTTGATGAGTCGCTTTATTTTGCGGAGGATCA
GTTATTAATTACCCAGTTCTTACTGAAATATCGGAGCTATGGCTTGATTG
CGGATGCGGGGTATTATTACTATCGTGATCTAAAGCAAAAAGGTTCTTTA
GTAAGTTCGTCTTGGAAAAAACCAGAACGATACACGCCTTTTTTGCAGAA
AGTATATCAAACCTATTTAACTGACTCGAAAGAAATATATGGCAAAGTGA
TTCCCTATGTGAAGTATCTAATTGCGTACCATGCAAAATTGTTTTTTTAT
AAAGAAAATACTTATTTCCGGGAAGTTTTAAGCGAGTCGGAGCAAGCGAT
TTTCGTGCAAGAATTACAAAAGATTTTGCAGGAAGTCGGAGCAAGTACGA
TTATGGAATTAGACACACCTCTCGTAGTAAAAGAAATGATGTGCTCGATT
TTGCAAAATGGATGGCCGCTGCAATTTGAAACGGCTGAAAAACAAGATAT
TCCACTTGTGACAGTAAAAGAAAATTATCGTATCGGCAAAACAGTCGCGA
TTGAATTACTTCTAGAAGAAGCGGATATTCATGACGTGAAATGGGTTGCT
CGCACGTCTTTTAAAGAAATGCCAGCTCGGTTAGTCAAACGAAAAGAGAA
TCAAACTATTTGGGATGTCGTTGTCAGAGAAAAAGGCACAGTAGAAAAAG
CTGTATTTAAATTAAAACCGTATCAAACGAAAGCGCGTCTTTTTTATCAA
GATGAGGAAAAGGATACGCCTATCGCCGACATAAATATCATAAGTAGCAT
CTTAGGGAAATTAAAACGAAATCGAGCGTTAAAGCGAAAGTTTAAACAGG
GGGGAGTATCCTGA
ORF1813 (SEQ ID NO:34)
CCTCGGATGCAAAACGCCTTAGGAAAAGCGCTAAAAAACAACGAAACAAC
ATTCAATTTTGACGAATATCAAAAAACGTACCGTGGTCAAATGTGGGTGC
TAATGAAAAATGGTATAGTAGATGTTGAAGCAACGAACGCGTACAATGCC
GCCGTGCTTAACGGGGAATTACCACATGAAAGCAATGAAGCGCAAGAAGA
AGCCGAGTTGTTGCAGGCAGTTATTCAATCGGTGAAAGAAGGAATAGACC
CGGTTACTGGGCAAGAGATAAGTAAAGCGCAAGGGTTTAGTATTATAAGT
GGCTTTATCTTTTACTACACTTCTGGTGGGTATAAAGGAAAAAAATTGAA
GATTCCTAAAAAATTATTAAACAATATTAAGAATAGGCGTTCGAAAGCAC
CCAAACTATCTACTTTATCCGAAAAGGAACAATTGAAACTAGCAAATAAA
TACAAGAAAAAAGCACCAATCGCAATTCCAGATAACGCTAAAATAAAAGC
CCAGACAAAAAAAGCTGGCTATGAACAAATTACTTATAAATGGAATGATG
GTCAATTTAAGTATGAAGTGCGATGGCATACAAGAACTCCAGAAGCACCT
TTAGAGCAGGGGAATACATGGGTTGTCCAAAGAACAGTTCCTGGAAATGG
AGGGGACAAGCCACAAACATTCTTCAAAATTGGTGATGATGAGTGGGTAG
AAGGATATAAATGGTATGATGCTATAAACGCAAGAAAAGCAGGAAATGCT
ACTCCAGAACAAATAAAAATTTTAGATAAAGGTCATTGGAAGGAATGA
ORF1849 (SEQ ID NO:35)
AATGAAAACAGCTCGGGGACGCCCTCAAGCTGTTTTTTTATGTTTCTGGT
GATAAGAAAATGGGAACAAGATACTATTAAACTTTTTGTCAGGGGGAGCA
AGATGAAATCCAAAGGGAGACTGTTTTTGTACGTTGTTTTGGCGTTGTCG
ATTGTTATTGGGACGAATGTATTTATAAAAATAGACGCACATGCAGCCGC
TGCACCACCAGCTGCAATCAGCCAGATTTTTCCCGATGATGCTTTAGCGA
CAGAAATTCAAACTACACTCGGAAAGTCTAGTACAGCAGAGGTTGTCACT
CAAACAGATTTAGATACTATTAATTCATTAACCCTTACTTCTAAAGGTAT
CAGCTCCCTAGAAGGTATGAACTACTTGACCAATTTAGGAACTTTAATTT
TAACTGGTAATCAAGTAAGCGATATTTCGCCACTTAAAGGCTTAACTAAC
TTGACCATGCTTCAGCTGAGTGGAAATCCGATTAGCGACATTAGCGCACT
TAGTAACTTGAAAAACTTACAAGCTCTAGATATTAACGACGCGCAAGTTA
CTGACATCACGCCACTGAGCGGTTTAACAAATCTAAAGGGTTTAGGCTTA
TATAATAATCAATTGGAGAACCTTAGTGGAGTAAATAGTTTACATCAACT
CCGCTCCTTAAATGTTAGTAATAATAAGCTTACGAACCTAGATGAGCTAC
AAGCGCTAAGCAATTTAAGCGTTCTATATGCAAATGAAAACCAAATTAAC
AATCTTCAGGGGCTAAGTAATTTAAATAACCTTTTCTTGCTTGATTTATC
AGCGAACCAAATTGTGGATACTACTCCACTCGCTGGCTTAACTAAGGTAC
AAACATTATACGTATCAAACAATCAAATTAGTGATGTAACTGGTCTTTCG
AGTTTAATAAATCTAGATTGGCTCGATATAAGTCAAAATAAAATTAGTAA
CATTAGACCTTTAAACAGTTTAACGAAGCTAACAATTATTCAAATGACTA
ATCAATTAATCGTAAACGAACCGATAAGTTTCGAAAGCACTGTAACGATT
CCTAATTTAATAAAAAACATTGCGGAACAAACGATTGATCCCGATACAAT
TAGCGATAACGGTGTGTATGCGAATGAAGCTGTTACATGGAATTTACCTA
CTTATATTCCTAAAGTCAGCTATACCTTTATTGAACGTGATACGATTGGT
AATGCAACTGGAAATTTCAGTGGGACTGTAGAACAGCCATTAGTACAATA
TTTTAAAGCAACTTTCAATATTGACGGGCAAGAAACAACGGAAAATGTCG
AAACAGGGACACTTTTACAAGAACCACCAACCCCAGTCAAAGAGGGTTAT
ACATTTAACGGATGGTATGATGCTGAAACAGGTGGAACAAAATGGGACTT
TACAGCCGATACGATGCCAGCAAATGATATAACGTTGTATGCCCAGTTTA
GTATTAATAGTTATACCGCAACTTTTGATGTAGATGGCGTCATTTCTACT
CAAGCGGTAGAGTATCAAGGTTTACTGGAAGAGCCACCAGCGCCAACTAA
GGATGGTTATACATTTAAAGGTTGGTATGATGCCAAAACTGGTGGAACAA
AATGGGACTTCACAAACAATCAAATGCCAGCAAATGATATAACCTTGTAT
GCCCAGTTTAGTAAAGATGCATCTTCTGGTGGGGACGGTGGAGGAACCGA
TGAAGGTGGAGGTAACTCTGAAAATAGTACAGAGGGAGCTCCTAATACGA
GTGATATAGATACTATAAACCACATCGTACTACCAGCAACAGGAGATGAC
CATGTGCTTTTTCCAATCTTTATTGGAACATTTCTTACGAGTTTAGCATT
ACTTACACTTAGAAGAAAATAA
ORF1912 (SEQ ID NO:36)
AGCAGAAATGCACGATATAGAGACCAAACAAGCCTCCTTGCGAAAGGAGT
GGAACCAATGAGTCGAATCGACATTGCAGAACTCAATGGCTTTCTTCAAG
GATTACGAAGCAGTAATGCTGAGGCTAAAAAGATGATACGCGGAATTCAA
CAAGCAGCAAGCAAGTATGCGCAAGATAAAAGTTTAAAAGGGCAAGCAGT
ATCAGCTTCCCAAAGCTATTTTGCTTCCAGTTATCCCTCTATTGCACAAA
GTATTTTAGAAGCTCTAGAAGAAAGTGAAGAACGTCTAGCACAATACATA
CGCGAGTTTGGTAGCCAAGTCGACAGCTCGCCTTCCGCACGAATTGATGC
GGAAATCCTTCAAGAAGCGATGGCGAAAGTGGCGAGTTTGAAGCGAAAAG
AAGAAGATTTACATCGCCAACTCACTGCACCGAATACGAAACCGGATATG
CAACAAGTCTACGTAGTCCAAGCAAGAAGTGCGCACACGCAATTACTGCA
AGCTATCGAAAAAGAAGATATACTAGAGAGGTACATCGCCTTTGAACAAA
GTCACGCCCAGTTTTTTAGTGCGTTAACCGAACTCATTCACTCCACAGGA
CGAGCGGTGCAAGAGTTGAAACAAAATGTGACCTTCCATGAGAAAACAGG
CACCTATGCTGTACCTAAAAGCGTTCATGCATCGATTAGTTTGATGAGAA
AAGCGATGGATAAAGCACGCAAAGAAAACGCCAAAGATTCCTTCCCAGAA
GCATTCGAAGACTATCAGTTATTCGCCTATACTTATGTGAATGACAAAGG
CGAAACCGTCACGATGTGGCTATTGGAACGGAATGGCAAACGTGCCAGTA
ATAAAGAACTCCAAGCTTTTCTAGAGGAGAACGGAGCCGAGTTAGACCCT
ATTCTGTACACAAATCTTTCCGGTGATGAACTAGAGCGCAAAGTAAATGA
TGCATGGAAAGACGGCGTCAACTACTTAAATGGTCAAAAAGTAACCGGGT
TTTCCGAGGCGACACTGAGGTCCTCTGCTTACGTAGCTAGCGTGAAGGAT
GCGATGGATGACGCTGGACTAACCGATATGGCATTAGGATTAGGCTTTGG
TATTGCGGCGGCGAGGAATAAAAAAATAATAGCTAAGAAATCAGAAAAAG
TAGAAGTTGATCTTGGAAGATATTCTGTAGCAACCTTAAATAGAGATCAA
GCACGATTAGTGGTTGAACATCTTTTAGAAAGAGGAGAAATAAGCTTAAA
AGATTTGGGTGATATAATACCAAAAGGAACACCAAACACTTTTAAAACTA
CAGATACCTTGAAGAATGGTGCTAAGTATGAATTTCAATTAGCTGATGGA
CAAAAAGCGATAATTAGGTGGCATGAACCAGACCCTATTGCAGCTGCTAA
ATTTCCTGGTTCAGCTTCTGGTTCAAGATGGACTGCTCAAATTAAGATAG
GAAGTAAACAAGTAACAATAAATAAAGGATGGACAAAAAAGCAAAACTCA
AATGAAGTGCATATTCCAATAAAAGGAAGGTAA
ORF1913 (SEQ ID NO:37)
ATGGACAGCACGCACACCCGGTTAGAACAGCAACTACAACAAGTGAAAAA
AGCGCAGGATGTTTTACAAGATAATCTGGGGCAGACTAAGCGCAAACAAG
TGGAACAAGAATGGTTAGAAGAAGATAGTCACCAATTAGAAATGGAGAAA
CAAGGATTATTGGACTTCTTGCGAGGCGGTTGGCAAGGGGAGGAAGCGAA
TGGTTTCCACCGCTATCTAGAAGAACAACAACACGAAGAAGCGATGGCAT
GGCGAAAAGATTTGTCGGAAAAACGTGTTCATTTGGAAGAAGAAGCGCGG
ACTACTAGAGCAGAAATGCACGATATAGAGACCAAACAAGCCTCCTTGCG
AAAGGAGTGGAACCAATGA
ORF1998 (SEQ ID NO:38)
GGCGGTATTTCGATGAAAAAATTAGAAAAAAGCGTACCAGTAATCCGGTT
TAAAGGCTTTTCTGAAGCTTGGGAACAGCGTAAGTTAGGTGAAATTGCGA
ATTCCTTCGAATATGGGCTTAATGCAAGTTCTAAAACATATGATGGAGAA
AATAAATATATTCGAATTACAGATATTGATGAAAGTTCTCATGTATTTAA
TCAAGATAATCTAACCTCCCCAGATATAAGTTTAGATAACCTAAATCATT
ACTTATTAGAAGAGGGTGATATTCTCTTAGCGAGAACAGGAGCAAGTACT
GGAAAATCTTATTGTTACAATAAAATAGATGGCAAAGTATTTTTTGCTGG
GTTTCTAATAAGAGCAAAAATCAAACATGAATATAATGTCAGTTTTATCT
TTCAAAGCACTTTAACAGAAAGGTATAATAATTTTATTCAGGTTACTTCT
CAGAGATCAGGCCAACCAGGAATTAATGCTCAAGAGTATGCTAGATTTGC
TTTATATATTCCTAAATTAAAAGAACAACAAAAAATAGGTGACTTTTTCA
AACAACTAGACGACACTATCGCTCTTCATCAGCGTAAGTTAGATACTTTG
AAACAGATGAAGAAAGGACTCTTACAGCAAATGTTCCCTAAAAGCGAAGA
GGATGTCCCAAAGATTCGTTTTGCTGATTTTGACGAAGAATGGTACCAGC
GTAAGTTGGGAGAGATTTCAGATAAAGTTATCGAAAAAAATAAAGAAAGT
ACATATTTTGAGACTTTAACCAATTCTGCCGAGTATGGAATTATAAGTCA
ACGTGAGTTTTTTAATAAAGATATCTCTAATGAAAAAAATTTAAATGGAT
ATTATATTGTCCGCGAGAATGATTTTGTTTATAATCCTCGTATTTCGAAT
TATGCACCTGTGGGGCCAATTAAGCGTAATAAACTAGGAAGAATTGGTAT
AGTCTCTCCTTTGTATTATGTTTTCAGAACATTTGATACAAACCAAAGCT
TCTTGGAATATTACTTTGATGGGACAGTTTGGCATAATTTTATGCTATTG
AATGGTGATTCTGGGGCAAGAGCAGACCGATTTGCAATCAAAGATTCAGT
TTTAAAAGAAATGCCTATTCCATATTCAACTTTATATGAACAAGAAAAAA
TAAGTTTTTTTTTAGATGAGATAACTATCATTATAAATCTTCATCAAAAT
AAACTAAAGAAACTCTCTTCTCTAAAAAAAGCCTATTTACAAAATATGTT
TATATAA
ORF1999 (SEQ ID NO:39)
GAGAAAGAAGGAAAAAACATAATGGCATTATCAACTGAACAAAAAACAAA
AATGTGGGCAATGCTCAACCAAACACGCGGTCAAATTGGTTTAACTGCGT
ATAAAGACTATATCTTTGGAATTTTGTTTTATAAATACTTATCTGAAAAA
GCAACACATTGGCTAAACGGCGTATTACGCGGGGAAAATTGGGAGAGTGT
TTATTCTCAAGATTCTGTAAAGGCCTTAAATTATATGAAAAAGAATCTTG
GATATGCGATTCAACCAAATGAATTCTTTGTAGACTGGAAAAAAGCAATT
GATACGGACCGTTTCAATATTGGGATGATGACAGATACATTTACGCATTT
TAATCAACAAATTGCATTTGAAGCGAAAAATGATTTTGAAGGAATTTTTG
ATGGTATGCGCTTTGATAGTGCGGATTTAGGTGCTAATGCACAAGCTAGG
GCTAGTGTCATGATTTCTATGATTGAATTGCTATCCTCTCCTGAATTTGA
TTTATCTGGTAGTAATGATACTGTTTCAGATATTTATGAGTATCTAGTTG
CACAATTTGCTACTGTCCTAGCATCTGATATGGGACAATACTATACACCA
AAAGAAATATCTAATGTAATGGCTCGGATTTTAACTTTTGGCCGAGAAGA
TATGGAAAAATTTTCTATCTTTGATCCTACTGTTGGTTCTGGTTCACTTT
TACTTACAACCGCTAGTTATATGAAGAACTCAGGTAGACGCGGGGTGATT
AAGTATTATGGTCAAGAAAAAGATGCAACACCTTACCGTTTATCAAGAAT
GAATTTGATGATGCACGGTATTGAATATAATGATATTAATATTAATCATG
CGGATACACTTGAAAGCGATTGGCCAGATGGGGTAGTGGATGGAAAAGAT
ACTCCTCGAATGTTTGATGCAGTAATGGCGAATCCTCCTTATTCAGCACA
TTGGAATAATAAAGATCGTGAAGATGACCCAAGATGGCGGGAATATGGTG
TTTCACCTAAGACAAAAGCTGATTATGCCTTTTTATTACACTGTTTGTAT
CACTTAGAAGATAATGGACGCATGGCGATTATTTTACCACATGGTGTATT
ATTCCGCGGAGCTTCTGAGGGGCGTATCCGAAAAGCTTTAATTGATAAGC
ACCAGATTGAGGCAATAATTGGTTTTCCTGAAAAATTATTCTTAAATACA
CCAATACCTGTTTGTGTAGTTATTTTAAGAAAGAATCGTATTGAATCAGA
TGTTCTGTTTGTTGATGCGAGCAAAGGATTTGAGAAAATTAAGAAACAAA
ATAATTTACGTTCTGAAGATGTGGAAAAGATTGTTGATACAGTAATTAAC
CGTAAAGAGATAGAGAAATACTCACATGTAGCAACTTTGGATGAAATAAA
AGAAAATGATTACAATTTAAATATTCCTCGATATGTGGATACTTTTGAAG
AAGAGGAGGCTATTGATTTGGTGGCGCTTGGTAATGAAATGGTTGCATTA
AATGCTGATATTAAAAAAGCCGAGACTGATTTTCTTGGTTTATTAGATGA
ATTAGCTGTTACTGCGGATACGAAGGAAATAATTGAAGCTACTAAGGCGG
TATTTCGATGA
ORF2013 (SEQ ID NO:40)
ATTATGAAAAGCAAAACAAAACAGATTATCATGATTGGAGTGGTCCTTTT
TCAATCACTCTTCGCATACCCGTTAATCACCATGGCGGAAGAAAATGAAT
CGAAATCAGTAAATACAGAAACCACGTTAGAGCCTAAAGTAGCTCTCGAA
GAAAAAACGCCTCAGAAACCTACCCTTACCAATAATCTGAAGCAAGAAAA
AACTGTCCTTCAAGCAGGCGAAACATATGAAACTGTTTTTCCTGATGCAG
CTTTAGCTACTGTAATTGCAAAAGCAGCAACTGGTTCAGAGGATATCACG
CAAGAAGTATCGCAAACAGACTTGAATAAAATCACTTCACTAACTGCTAC
ATCTAAAGGGATAGTTGATTTAACAGGAATAGATTTACTTTCAAAATTAA
CCTCTTTAAGTATAAGCGGGAACCAAATCACTGATATTTCTGCACTCAAT
GGTCTCGTGAATTTGTCCAATCTAAATGTATCTAATAATAAAATAACAAG
TTTCAACCTAAACGCGAATAGTAATTTACCTATGTTAAGCGCTGTTGATA
TTCGTAGTAATAACTTAAAAAATATAAATGTTCAAGACCAACCTAAATTA
CGCACCATTGAGTGTGACACAGGTAGTAGTTCAGAGTTGACAGAAGTTAC
GCTAAAAAATCTTCCAATTTTGATAGTTGCAGGTAATGGCTCTAGTGCTT
ATCAAAATGATATTGTTTTTTCGAGTACACCAGGATTAAGTAAGGTGATT
CTAGAAAATTTACCATCAATAAGCTCTTCAGTACGATTAGATCGTTGCGC
GATAGAAGAGTTAGTAATTAATAACCTTCCAAAAGTATCAATGGTAAATA
TAAGTAACAACAAAATTACTACACTAGAAGGACTTGAAAATTTATCTGCA
GTAAACACTTTATACGTATCTGAGAATTTAGTGACTGAAATAGAGAACCT
ACATGCGTTCCCTAAATTACAGACACTCACCGTAGACAATAACCATATCA
GTGTATTGCCAACAAGTTTGAAAACGGAAAATCCCGTATTAACAACGCTA
AGTGCGATGAATCAAACAATCACTCTAAAGCAAAAAGTTATCGTGTCAGA
CCTAGTTCTTGATAATGAAGTGAAGAATTTCGGTCAAATAACCACTGCCA
AATCCATCTCTAATAAGGGAACCTATCAAAATAACCAAATCAAGTGGCTT
TTTGAAGATATAAAAAGCGTGAATGCCGTTGATTATCAATTTAGTGAACC
TGTTCAAGAGGCAACTATTCAAGGAACTTTTTCGGGGAAAGTGACACAAC
CAATCAAAGCATCTAAAGTACCAGTTATTAGCGCAGATGCAGAGATGAAT
TACCCGAAAAACGAAACGGTATCAGAAGCTGCCTTTTTCAAAGATATTTC
TGCAAGCGTAACGGATGATGCAACACTAACTTCTGATTTTGAAAGTGTTG
TGGACTTTGCAAAAGCGGGAACGTATGAAGTGACATTAAATGCAGTGAAT
GAGGATGGAGTAAAAGCGACTTCGGTGACTGTATTAGTGCATATCGCTAA
GTCGCCAGCGCCAGTAATTACCGCAGATAAAGAAATCACATACACTAAAA
ACGCGGAAGTCAGCATCACGGAATATCTTGCAGCGATTCATGCTAAAACG
AATGATGGTTCACCAATTGAAAGTGATTTCGCTACGGCTGTAAATTGGGG
CACTGCAGGAGATTATACCGTAACGCTAAGGTCTACAAATGAAGATGGAG
TTGAAGCAATCCCTGTAGAAGTAACTGTGCACATCGCCAAGTCGCCAGCG
CCAGTAATTACCGCAGATAAAGAAATTACGTACGCTAAAAACGCGGAAGT
CAGCATCACGGAATATCTTGCAGCGATTCATGCTAAAACGAGTGATGGTT
CATCAATTGAAGCTGATTTAGATACGGCTGTAACATGGGGCACTGTAGGA
GGTTATACCGTAACGCTAAGGTCTACAAATGAAGACGGAGTAGAAGCAAT
CCCTGTAGAAGTAACTGTGCACATCGCTAAGTCACCAGCACCAGTAATTA
CCGCAGATAAAGAAATCACGTACGCTAAAAACGCGGAAGTCAGCATAACG
GAATTTCTTGCAGCGATTCATGCTAAAACGAGTGATGGTTCACCAATTGA
AAGTGATTTCGCTACGGCTGTAATATGGAGCACTGCAGGAGATTATACCG
TAACGTTAAAATCTACAAATGAAGATGGAGTAGAAGCAATCCCTGTAGAA
GTAAAGGTGCATATCGTAGAGCCACTAGCACCAACGATTTCGAATGTGAC
ATTTGATGTGGATGATGTACAAACGACAGAATCTCTTGAAGCTGGAGAGC
TAATTTCTGAACCATTGAGCCCAACAAAAGAAGGCTATACTTTTATTGGT
TGGTATGACTCGAAAACTGGTGGTAATAAATGGGATTTTACAACAGATAA
AATGCCAGCATATAATATTATTCTTTATGCTCAGTTTAGTAAAGATACAA
ATAAAGCAGAAGCGGCCGGTGGAGATAAGCCCTCAACACCCTCTTCTATA
AAAGTAAGTCCAACAGGTCAGTCCGAGAGTGGAAACTTGGAAAATAGATC
TAATATTAAATTACCAGCTACAGGCGATGATAATGCAACTGTTTTATTAG
TGGGCTTTGGATTACTAATGTTGGGGCTTTTCATTCGCCTTACTCAAAAA
AAGCGTGCTAAATAA
ORF2017 (SEQ ID NO:41)
ATTATCTCTTGGAGATTTGTTAATAAAAACAACCAGAATATGAGAAGGAG
GAAAACTAATATGATAAAATCGTTGTACATACGTCGTTCCTTGATGATGC
TAATAACAAGTTTACTAGTAGTAAGTCAACTAAATTTAACGGTTTTTCAA
GCATTTGCAGAAGAAAAAGGCGAAGAACCACTTTCTTATGAAGTTCAAGA
AGAGCTTTCAAAAGATAAAAAGAAAGCAAAGCTTAAAATTAAAGCAACAT
CTAATAATAATCAAGTTGAAATTCTAAGTATTGAAACACCAGACGGTAAT
AAAATTGAGGGAAAAGAAGCCGAGTATACGGCTGAAAAAAATGGTGATGT
TAATTTTATTATTAGCTATAAAGAAAACATAGAAGGAAAAGAAACAGAGA
TTAAAACATTTAAAGCCTCCTATGAAGTAAAAAATTTAATAATTGAAAAA
GAAGAAGCTAACCAATCAAAAGATTCAGTTAAACAAAATGAAACAAAAGC
ACCTCTAAAGAATAATCAACTAAAATCAGGTAATGATGCTACTGTCACAT
TAAATATTCCTGCTTATGATGGTACTGCTTGGGCAAATGGCGATATCAAA
GATGTTAATGTTACAGTAGACTTTAGTAATAGTACATCTACTAGTAAAGA
GGTGGAATTCACACTGCCAGACGGTATGAGATTTGTATCAATTCCCGTTC
CCAGCAGTTTTCAAGTACCAACAGGTGTGGACTCTGGGGTGATAAATCGT
TTAAGTGCGGGGGACCCGATTGGTACTGCAATAACATCCGTTACTATTCC
AAACAAAGAAACAACCTACGATAAAGCTACTTTCGGGACAGTTAATTATA
AACTTACTCCAGGAACAGAAAAAGTAAGTTTTAACTTTTCTGTTCGTGTA
GATGCTAATAAATACTATGGTGCAACTGATCTAAAAGCCCCTATTAAAGT
AGAAGCTTTTGTGGGAGATAATAAAATGGCGATTGCTTCAGAAGAAAAAG
CAATTCGTGCAGAAGGAAATAAAGTGGTTGGTTATGCTAATCAAACACAT
GTAAGTACTATGTTCAGAAATTGGTATAAATCTTTTAGAACACCTGATGT
ATTAGCGAGTACAGATGATGAAGATTCTTATGCATATACAAAAAGTTACT
CTGTTGTAAATGGAATGAATCAAGTAGATGAAAGAGGAACTAATATCTTT
TCACCTAAGAATGTCTCGACTACCCTTTATTATCCAGAAGGTATGGAATA
TGTCGGGGTAGTAAATGAAAAATATGCACTTTTAAATGAGAATGCTAATA
CTACGATTACTCATTATCCGGAAGAGAATAAAGTGGTGTTCGATTTTAAA
CAACAGAACTTTAAAGGAGTAACAGATACGGTATATGCGATAAAATATAA
AATACCAAAAGGAACGCCAGCAGGAACATATACCGCTCCTAAAGTGCCAC
ATGCAGTCATCACGACATATGACGATAAAGTATTTGAATCTGATGCATTA
ACAAACAATTCTACAGATTTAACAACTAAGACAACGTTAGATGCTTGCCG
AGTTGTAGATTCGACCGAAAATAAAATGGCACTAATCGTGGGAAACAAAA
ATATTAATCCAGATAATGAAACTTGGGCAGGGAATGTTCGAATTAATAAT
AAAGATACTGCCGGGACAAAAACTAATCAAGTCTTCCAAATTGAATTTGA
TTCAAACTGGGAAGCTTCAATTGTTAACATCCCATTTGATGGGAATAGAA
AAGATAATGATATCACTAATATTCAATACAAAACTAATTTTGATGATACT
TATCAAACATACAATGGGAATCTTCCTAAGAATAGTATGAAAAATATTGC
TATCTTAGAAGCTGATGCAGTCGGACTTCAACCAGGAGAATACTTTACTG
AGGTGAAAGCGAATGTAGGCGATTTTTCTACAGGATATACAAGCTACTCA
GAATCTGGTGGTTTTGGTGAGGAATCAACAAACTCCTATGGTATTGTCAA
ACCAGGCATTTCTTCTGTTCAATTTAAAGTATCCATTTGGGATGAGGCAG
ATGAAGTAAATACGAAAGTCTCGGGTACTTCTGTTTATAGTGTTGCCGAT
AATATTACGACAGCGGCGAACGGAACAGCTAATTTTTATAATGAACAAGG
ACAAGTAATCAAAACTGCTGGAGCTGGAGATAAATTTACTACAAAAGCTA
CATTGGTATTATTTAACTATCCATACGGGACACGTACAGTCCTTAATGAT
CCTGAAGTGTATCTTCGTCAATTGGATGGTACTAAAATTCTTCCGTCTTC
TATTAAGTTAACGGATCAAGATGGAGAAGAGATCGAGTTCTCTATTAAAA
TAGAAACCGCCAAGAATGGAGAGAAAGTGTATGTTTTAAAAACAAAAGAT
GCAACAGTTGGTAGGTATGTTGGTTATCCTTCTAAACATAAATTTTTAAA
TATAAGTTATGATACTACGATTGATGTAACATTAGATAAAAGTCTTCATA
CTGACATCCAACAACTTCTTGCTTGGGGAGGCCCAAATGTAAAAGCTGCT
ACAGCTGCGAATACTTTCATGGATACAGGTCTTGATGTCAATCAAAATGG
AATAGAAAATGAACGTCTGCTTTCTGCGAATAGCAGTACTTTAAGTGTTC
CAAAGCAAGATGCAGTCACAGTAGAAACATTCTTAAATTTGGCTGGAGAA
GGAGCAAAAGCATCTTATGTAGAAGGTGACGATAGTACAGTTTCCTATTT
TACACCAGGAACGGAAGCGGATTATACAGTCCGTGTTACAAATACATCTA
CTGGTATAGCAAGTGCTTTTGAACTCTATATTCCAATTCCAAAGACCGGA
CAAAATTTTGGTTCTAAATTCCAAACGGAGACATTTAAGTGGGATATGAA
ACTAAATGATGCTGTGCAATTAACAGATGAACAAAAAGCACAATTTGATA
TTAGTTATGCAACAGAAGCAAATAAAAATAACTATGAATCAGAAAGTGTC
TATAGCGCTGTTCCCTCTGATTATGGGAAAGTCAACATGGTTCGTATCAA
AGTGAAGACAAAGATTGATCCCGGTGAAACACAAACAATCAAAGTTCCAC
TAAAAGTAGATGAAACTTTTAATTCTGCTACAGAGGGAAATAAAATAAGT
GAACGCGATGTATATAACCCTTACTATCGTGTAACTACGAATGCTTATTC
TGGAACATTACCAGGAACTAAAGTTGGAGCAGAATTAATTATTTTAGAAG
CAAGCGGTTTTCTTTTCCAAGATAAAGATGCAAATGGTTTGTATGAAAAA
GCTCAAGGAGATACAGCACTCGCAAACGAAACTGTTGAACTTTATAAATG
GAATGAAAATACCTCTAACTATGAGCCGTTCTTGAAAAATGGTGAGCCAG
TCAGCGTTAAAACTAATGCTGATGGAAAATACACCTTTAATTACAATTTG
AATTTATCTTACGGGAAATATGCAGTGAAGTTTCCAGAAAGAGCTGGAAA
TCAGTTTACTTTGAAACAAGTGGGGCAAGATAATACGATAAATAGTACTG
TATCCAATAAGGGTACAGACAAAGGTTGGGTAAAAGAAATTGATCCAGCT
CAACCGGTATCACAAAACATTAATGCTGGTTACATGGAATACGCGCCGGA
TAATGATCTTAAAGTTAATCTTAGCGCCAAAATCGTACAAGCTGGTAAAA
GTTTAAAAGTAACATTGCCGAAAGTAAGAGCAACTAGTGGAGAAGCGGTA
GAAGATACGATTGAGCCGTCGTTCTTTCACAATATTCAAGCGATTACAGA
TGGTTACAAATGGACTAGCAATGATACGACTTTAGCAACTACACAAACTG
CTAATGATGGTTCTGGAATAATTGTTGGTATTTCTGCCGGAAACAAAGCA
ATCGCAACAACTGATATTGGTATTACAATCAAAGATATTTTTGATACAGA
GAAAAAATCTACTGCACCTGTATATATAACTGAGCCAAGTGCTACGATAG
ACCAAAAAGAGGGACTTATCCTTGGCGCATTGAATTTTAGTTTAGAATAC
AAAGATTCAAATAAGCTAACTGAAACGGAAGCAATTACTTTAGCAAAAAC
AGCTAGTTTTGAAGAAGTGAAGACAGGTGTACTTTCTACTGCACAAGATT
GTACTAGTTCCGTAAAAGTGGATGCCAAGCAATTAAAAGCTATCCAAGAA
GGCTCTAACCAAGGCGGAACTTACCCGTTGACCTATCAAGTTGCTAAAAA
TGGGAAAACAGCGGAAGTAGTTATTCAAGTGAAAGTTGAAAAAGATTTAA
CAACTGTCAATGTACATGATTCTATTTTGTATTTGGGTGATACATGGACG
GCAACTGATAACTTTGATAGTGCGCTAAACAAAGTAGGTGAAACAGTTCC
ATTTGGAGACATCCAAGTAGAAGGATCTGTTGATACTAATACAGCAGGAA
TCTATTCAGTCACATATACGTACAAAGGAGTGTCTAAAAAAGCTAAAATT
GAAGTTAAAGAAAATTTGACAGAAATTAATGCACACGACTCGACAATTTA
TACTGGCGACACTTGGCGAGCAGGTGATAATTTTGACAGTGGACTTGATA
AAGATGGCAATGCCTTGAGTCTAAAAGATTTGACGGTTATTGGAACAGTA
AACACAAACCTAGCAGGCGTATATACGATTACGTATAAGTATGAAGATAC
TGTAAGTTCAATTACTGTTACTGTAAAAGAAAATAAAAAAGGAATTAATG
GGCATGACTCGTCAATCTATGTAGGAGAAGCATGGACAGCGGCGGATAAT
TTTGATAATGCAGTTGATAAAGACGGGAAGCCAGTTTCTTTTGCTGATAT
AAAAGTGAAAGAAGAGCCTAAAGTAGATGTCAACAAAGCAGGCAGATATC
AAATCACCTATAATTATGATGGTGCCTCGACTACTGTTACTTTAACTGTG
AAAGAAATCAAAACAGCTATTAATGCACATGATTCTATTCTGTATATTGA
TGATAATTGGTCAGCAAAAGATAATTTTGATAGCGCTAGGGATAAGGATG
GAAATATAGTATCCTTTAATGACGTACAAGTCTTGGGAACTGTTGATACA
AGTCAAGCAGGAACATACCCGATCACATATGTTTATGCTGGAATTACCAA
AACCATTCAAGTAATTGTTAAACATCCCAAAACAGCTGTGCGAGCCCATG
ATTCAGTGATTTATGTTGGAGATAATTGGAAAGCTCAAGATAATTGGGAC
AATACGCTCGATAAAGCTGGTCAAAAAGTGAAATGGAAAGATATTACTGT
CAAAGAAAATCCTGCCGTGGATGTAACAAAGCCAGGCGTCTATGAAGTTA
CCTACAGTTATGACGGTATTTCTGCCACGATTAATGTAACCGTGAAACCA
AGAAAAACGACTGTAAAAATTCATGATAGTAGTTTTTATGCCGGAAATAG
TTGGAACGCGAAAGATAATTTTGACCATGCAACGAATAAAGCAGGTGAAA
AAGTAGCATTCAAAGATATTACTGTCGCAGGTAATGTTGATAGCAAAACA
CCAGGAACATATGAAATTAGCTATGTATACGATGGTGTGAAAGCTGTTGC
TAAGGTCACTGTTTTGAAAAACCATTCTATTTTGATGGTGAAAGATCGTG
TCATAAAAGTAGGCGAGAAATGGAACGCGAAAGATAGCTTTATTCAAGCA
ACTAGACGTGATGGAAAAAATATTCCATTTTCACAAGTAAAAGTGCAAGG
AAAAGTAAATCTGAACAAAGCAGGCAAATACCAAGTTATATATTCAGTAG
ATCCAAATGAAGGAACTGAAGACGCAGGGAAACAAATGTTGTCAGTAACT
GCTACTATTCAGGTTGAAAATAGAGGAGATGCGGGGTTACAAGATAATGA
ACCACGAAGCGTAAAAGATAACAATCAACGCGCTGCTAACTCCAACGATA
TCAAACTCATTCCTAAAACAGGAGATCAAACAAATCAGTGGGTTTTATGG
GCTGGCCTTTGTTTGACTGGTTTAGCAATGCTTCTATGGGGATTTGCTCT
AAGGAGACGAAAAGTGAAATAA
ORF2019 (SEQ ID NO:42)
AAATTAACTACGAAAGAAGGAGGGGAGCTGATGAAGAAGTTTTCCATGCG
AGTGGTGCTTATTATTTCTGTGCTTTTTATTGCACTGGGGAACGCTAATG
TTTCTATCGCACAAGAAAGGGACACAACGAATAAGCTTCCAGAAGAGGAG
CTGGGTTCTCTGGATACCTCTAATCTTATTGCAGAAGAAGTTGCGCAAGA
GAAACCAGCCGAGGTCGAGAATCTAGAAGAAATCCCAACTACGGATGAGT
TGATGCAAAATCCAGAGGTTCTGGAGAAGTCTGTTGCCGACTCGGATGAT
CCGGATTTAACAGTGGTAAACTCGGGTGCTTACTGGACGCTTTATTATAA
TTCTGCGAACGGTGAATATAGCTTGCGTATGTTTGGCAACGTACCAAGTA
GTATACCAAGCGCTTGGAAGAGTTATTTGGGTAACATTAAACATATAGAG
ATTGAAGAAGCTACTTTGACAGGAAGTTTTGAAGCTTATTTTAAAAGTAA
AATAGATGGTTTCAGAGTGCTTGAGAGCGTGAGAATTGAACGTTCTAATT
TGTCTGGAGTAACATCTTTTAGAGAAGCATTTTTTAGTGCAGGAATTGAA
AAAGTTATCATTAGAGATAATGATTATCCGACAGCACCATCTTTACTCAC
TACGGAAAGTATGTTTGAAAATTGTTACAACCTTACGGAGTTTGATGTGA
GTGGTCTTGATACTAGCGCAGTAACTAATATGAAGAAAATGTTTTATAAC
TGTAGAACACTTGAGGAATTGGATTTAAGCAACTTGGATACGAGTTCCGT
TAATAGTATGAATAGCATGTTTGGATACTGTGAATCACTTGAGAAATTGA
ATGTAAGTAATTTTGATACGAGTTCTGTGAATGATATGTATCATATGTTT
AGGGATTGTAAAGCACTTGAAAAATTGGATGTAAGTAATTTTGATACGAG
TTCCGTTACTAATATGGGAGGCATGTTTGTTAATAGTACCAGTCTTAAAG
AATTAGATGTAAGTAATTTTGATACGAGTTCCGTTACTAATATGTCAGCC
TTGTTTAGTTCCTGTAGAGCACTTGAGAAATTGGATGTAAGCAATTTTGA
TACGAGTTCCGTTACTACTATGTTGGCCATGTTTGTTGCTTGTAATAGCC
TTGAAGAGTTAGATGTAAGTAATTTTGATACTAGTTCAGTAACTACTATG
CAGTCCATGTTTTTTGAGTGCAGCTCACTTGAAGAGTTAGATGTAAGTAA
TTTTGATACGAGTTCAGTAACTACTATGCAGTCCATGTTTGAAAAATGTA
CTAGCCTTGAAGAGCTGGATTTAAGTACCTTTGATACGAGTTCAGTGACT
AATATGCAGTCCATGTTTATAAATTGTGCAGTGTTAAAATCTTTATATCT
GGATAATTTTACTACCGCAAAGACCATGACAGATATGTTTACTGGAACAA
CCTCCCTAGCCTACTTGTTTGTCAGTCACAATTTACAATCTTTTTATGGG
TTGGCAAATACAAACTGGTATGATGAAAAGAATTGGGTACAGTTTTCTAA
TCTCTCTCAACTACAGACCTATCATCGAAATCAAAGTGAACCTACTGGCT
ATAGAAAAGGTACCTTCCTTTCCCTAACAATGGATGCGATGGGTGGGCAG
TTTGATGATGCGGAGGAACAAAAAGTACAAAGTAAAATTTCTGGGGAATA
CTGGGAAGAAGTGATTCCAGTAAAAGAAGGACATTACTTTGACGGGTGGT
ATCTTGATCAAAATTTCACTAATAAGTTTGATTTTTCTTTACCAGCAACT
GTTTCAGCAACCCTCTACGCCAAATGGGTTGAAAATTATACAGTGATTAT
TCCAGCGTCTATTTCCTTAAATGAAGCTACTGAATTGAAGGTAGAAGGGA
TTAATCGTGGAAGTAAAACTTTGTCCGTGGGATTAAACCGAACAGCAACG
TCTGTTTCTGAGAGCAACAAGCTAACTTTGGCCAATACAGCAGACACAAC
AATCCAATGTTTGGCACCACTAAGTTGGGACGGTTCCGAAAACAATCCAA
AAAATGCCATTTTAACCCTAGCACCAGGTTCGGAAATAACAGAAGGCGAT
GCAGTCATGGCTATTGAAGCACCAGAGAATATCCAGGCAGGAACGTATAC
AGGGAATCTGGTATTTTCGATTAATTATGAATAA
ORF2074 (SEQ ID NO:43)
AGGATAGGAGATAGATTAATAGTGGAAATAACAGAAACGAAAGAATGGTG
GAAAGAATCGGTAGTGTACCAAATCTATCCGCGTAGTTTTCAAGATTCAA
ATGGGGATGGTATTGGAGATATTAGAGGAATTATAGAACGACTTCCTTAT
TTGAAAGATTTAGGTATTAATGTGATTTGGCTTTGTCCTGTCTATAAATC
ACCTATGGATGATGGTGGTTATGACATTTCTGATTACTATGAGATTGATC
CAATGTTTGGAACGATGAGTGATATGGATGAATTAATTGAGAAGGCAGAA
AAATTAGGAATAAAAATATTGATGGATTTGGTGGTAAACCATACATCTGA
TGAACATGAATGGTTTGAAAAAGCAATAGCTGACCCAAAAAGTAAATATC
GAGATTATTATATTTTCCGAGAGGGTGTAAATGGAAACCCGCCTAATAAT
TGGCGTTCTTATTTTGGAGGATCTGCGTGGGAAGCCGTACCAGGTGAGGA
AAACATGTTTTACCTACATGCTTTTTCTAAAAAACAGCCAGATTTAAACT
GGGAAAACATTGTAGTGCGTAATGAATGTATCCAAATGATTAACTGGTGG
CTTGAAAAAGGTTTAGGTGGCTTTAGAATTGATGCCATTTTAAATTTGAA
GAAAAGAATTGAGTACGGTACTTTCCCAGCGGACGGGGAAGATGGCTTGG
TTTTCATCGGTCATTGGATTTTAAATCAGCCGGGAATTGAAGAGTGGCTC
AAGGAGATTGACGAACGGACATTTAAAAAACATAATGCATTTACTGTGGC
TGAGGCGGATGTGCCTGAAGAAAGACTTTCTGAATTTATAGGTGAAAATG
GTCATTTCCGTATGGTGTTTGATTTTAGCTATACAGATATTGATACTCCT
GAAACAGGCGAATGGTTCAAGAATTCAGAATGGACAGTAAAAGAATTAAA
AGAGAAAATAATTACCAATGAACTGGTGACACAGCGAAATGGTTGGGGGG
CAAAATACTTAGAAAATCATGATCAACCGCGTTCGATAAATAAGTATTTA
CCTCAAGAATACCAAGATGATCGTAGCAAAAAAATGCTAGGCACGTTATT
TATGATGTTGCACGGTACGCCGTTCATTTATCAAGGACAAGAGATTGGTA
TGAGTAATACTCGGATGGAAAGTATAGATGATTATAACGATATTGCGACG
CACGATCAATACCATCGCGCAATTCTCTCTGGAATGAGTCCGGAAGAAGC
GCTTGAAGGAATGTATCGGCGTAGTAGGGATAATTCAAGAACACCGATGC
AATGGAATAATCAAAAGAACGCTGGTTTTTCCGACTCAGATGAAATTTGG
TTGAAAGCAAACCCTAATTACCTTGATATTAACGTAGAGCAGGAACAAAT
CGATGATAATTCCGTATTGAATTTTTATAAGAAATTAATCCATTTAAGAA
GTGATTCTAGTAAATATAAAGAAGTGGCTGTTTATGGAGAATTACTGCCA
GTAGAATCAAGTGATGAAGTTATTGCTTACAAACGTAAAACAGATGACGC
TGAGCTCTTAATCATCGTGAATTTTTCTGATTCTGAAAATCAATTATGCA
TTGAAGGTACTTATGAACAGGTACTTGCTAATGTAGCGTTGCCAGAAATG
GTAGAAAATGTCCTTGAAATACCTGCATATACAGGTGCCGTCTTCTCAAG
GGTTTTGGAGGTAGACTGA
ORF2075 (SEQ ID NO:44)
CATTCATTCTTAGCGCGGCATTCTCGTTATTTGCCTACACACGTACAAAT
TCTTATAAAGAAGGGAGCAACTGAAATGAAAAGAGCTACAGTATGGCCTA
ATCTATTGTTGATAGTGCTCGGGTGTATCTGGATATTCCCGATTATCTGG
ATTGTTTTGACAGCTTTTAGAGCGGAAAGTGGACAATTTGTTTCGTATAT
CTTTCCGAAAGAATATACGCTAGATCATTTTGCGTACCTATTTGAAAATC
ATGAAAATTTTCCTTTTATGTTATGGGTTAAAAATACAATGATTGTTGCG
GTGTGTTCCTGTTTATTGAGCACTTTTATAACCGTTTCGATGGCTTATGT
GTTATCTAGACTTCGCTTTAGATTTAAAAAGACGATGTTAAAAACAGCGC
TAGTTTTAAATATGTTTCCTGCTTTTATGAGCATGATAGCAGTTTACTAT
ATTTTGAAAGCATTTGGATTAACGCAATCTCTAACGGCGCTTGTGTTAAT
TTATTCATCTACAGCTGCATTAACTTTTTATATTGCAAAAGGATTTTTTG
ATACAATACCAAAATCTTTGGACGAATCGGCAATGATGGACGGGGCGACG
AAATTAAGTATCTTTACAAAAATAACTTTACCAATGTCTAAGCCTATTAT
TGTATACACAGCTTTAATGGCATTTATGTTGCCTTGGATGGATTTTATTT
TTGCTAAAGTGATTATGGGAGACAATGTGCCTAAATATACAGTGTCGATA
GGTTTATATTCCATGTTGAATCAAACAACGGCAAATACAATGTATACAAC
GTTTGCGGCTGGCTGTATATTGATTGCGGTACCAATTACAATACTGTTTA
TTTATTTGCAAAAATATTATGTTGAAGGCATTACAAGTGGTGCTGTTAAA
GGATAG
ORF2076 (SEQ ID NO:45)
GTTAAGTATATGATGGTTAAGAAGAATTATTTTAATATTTTTAAGAATGG
TTCTGTTTCTACGAGATTATCTTATGTAATTATGGGGGCTGGTAATTTGG
CCCATAAGCAAATTGCGAAGGGACTTCTATTTCTTTTTAGTGAACTTGCT
TTCTTGTTTTTCCTTGTGTTTTATGGCATTACCTTAATTCAAGGTATGGC
GACATTAGGGACAGTTAACCAATCGTGGAATTTTGATAAGAGTTTAGGTA
TTATGGTTCGAACACCGGGCGATAATTCCATGCTGATGCTAATATATGGG
ATTATGACAGTTGTTATTTGTGTACTATTTCTCTTCTTGTACCTTGCTAA
TATTAGAAGCGCTTGCCAGGTGGAGGGCTTCCAGCGGGAAAATAAAAAAA
TCCCAACATTTAAGGAAGACTTAAAGAGTTTGCTAGACAATAAATTTCAT
GTAACTTTACTGACGATCCCGATGATTGGGGTTGTGGTGTTTACGATAAT
GCCACTTGTTTATATGATTTCTTTGGCTTTTACCAATTATGATCATGAAC
ATTTACCGCCAAGGAATTTATTTGGATGGGTAGGATTTGTTAACTTTAAA
AATGTGCTAAATGGTGATATTTCCAGTACGTTCTTTCCGGTGCTTGGATG
GACACTTATTTGGGCGCTTTTAGCTACTGCTACTTGTTTCTTTTTTGGCG
TATTGCTTGCACTTCTTATTAATCATAAGGGTGTTAAATTCAAAAAGTTT
TGGCGCACTATCTTTGTTATAACTATGGCGGTACCTCCGTTTGTGTCACT
TCTCGTAATGCAGAACTTGTTGCATGCAGCAGGACCGCTTAATACGATGT
TACTTAATTGGGGAATTATAGCTGAGCCGATACCATTTTTGACAGATGCT
TTGCTAGCAAAGGTATCCGTCATATTTGTCAATATGTGGATTGGGATTCC
GGTAACTATGTTAATCGCCACGGGGATTTTAATGAATCTACCGAAAGATC
AATTGGAAGCGGCGCGAATGGATGGAGGTAACAGTTTTCATTTATTTAGA
TACATAACTTTCCCTCAAATCTTAACGGTGATGTTGCCAAGTCTGATTCA
ACAATTTATTGGAAATATAAATAACTTTAATGTGATCTATCTCTTGACTG
GTGGAGGACCTTCGAATAGTAACTTCTATGGTGCTGGGGATACGGATTTG
CTCGTGACATGGCTTTATAAATTAACAGTAGAAGCTGCGGATTATAATTT
AGCTTCGGTTATTGGGATTGTAACATTCATTCTTAGCGCGGCATTCTCGT
TATTTGCCTACACACGTACAAATTCTTATAAAGAAGGGAGCAACTGA
ORF2110 (SEQ ID NO:46)
ACAAAAAAAGGAGATGCAACTATGAAAAAGCTTAGAATATTTACTTTGAT
GCTTGCTTTTGCTCTATTTTTATTAGGTGGAGGAATTGTTGCACAAGCAG
CAGAGGAAGCCCCAATCGATGAAAAGATAGTTGGGGAAACGGTAACAAAT
GACGGAGAAGAGTTTATAGTCGATGAGATTAGTGATTTAGAAGATGTTAA
TTCAACAACGGGGACGTTAGATACATCTGATGAAGTTGAGGTTGATTTAT
CAGGATTAACTATTGAAAATGAGGAAGCCGTTCTAACCCCAGGTTTAAAA
ACGATTTTTGGCGGAGATGGTAGAAAACTTGTAACAAATACAACTCAATA
TCCATATTCAACGAGCGCTTACCTTGTAATGGAATTTCCAAATGGAAAAA
CTTATATAGGTAGTGGACAATTGATTGGTGAAGATTCTGTACTTACAGCC
GCTCATTGTTTATATGGTAAAAAAGATGGTGGATGGGCAAAAAAAGTGAC
TGTATATCCTGGATATAATGGCACGAAAGCTCCTTTTGGAACAGCAAAAG
CAAGAAAAATGTATGTTCCAAAAGAATGGACAAAAAAAGAACCTTCTACA
GAAGATTATGGTGTTATTAAATTAGATAAAAATATTGGGACAAAAACTGG
AACAATGGGGTTAACAACTAATACATCTGGTGCAATTACTATTAGTGGTT
ATCATGGTGACAAAAAAGGGAAATTGTACACTCAAACTGGAAATATCTCT
CAAGTCACTGCAAATAATGTTTTTTATAGATTAGATACAACAGGTGGTAG
TAGTGGTAGTGGTGTTTATAATTCTAAAAAACAGATTTTAGCAGTAAACG
CATATGAATATTTAAATGGTACCGGGGACAACTTTGGTACAAGAATAACA
AAAGAAAAACTAAATAATATTTATACTTGGGCGTTTGACAATAATCTTTC
TGTAAGCAAACAAAAAGGGATAAATTACGAGCTCCACGTCCAAAGTAAGG
GATGGATGGGAAACGTTGCTAATAGTATGACTTCGGGCACAGTTGGCCTT
GGCTTACGAGCAGAAGCAATGAAAATTAGTTTATCAGGGATGCCGTATTC
TGGAGATATTCAATATCGCTCACATGTACAAGGTAGTGGATGGCAAGGTT
GGTTGAAAAATGGGGAAATTTCTGGAACTCGTGGACAGTCAAAACGTCTG
GAAGCTTTCCAAGTAAAATTAACTGGAAATATGGCTAAAAATTATAGTGT
GCAGTATAGAGCGCATGTCCAAAATAAAGGTTGGCAAAGTTGGGTTAAAG
ATGGAGCAACAGCAGGAACAACAGGAGAAAGTTTAAGAATAGAAGCTGTA
CAAATGCGACTAGTTGCAAAATAA
ORF2192 (SEQ ID NO:47)
GCTAAATTTTTTATGGGAACTATGAGGTATGATATAGAGAAAATTACAGT
AATAGTTCATAACAAAATTGCGAAAGGACGTATTTATGTGGCAGAAGCAA
AATTTGAAGCAGCTTTAATTAAAAAGCTTGAAACTGAAGGATGGACTTAT
CGGGAAGATTTATCCAATGTTAGTATTAAAAAACTTGAAGAGCATTGGCG
TGATATCTTAAATGAAACTAACGCTCATAAACTCAATGGAGTACCATTGT
CAGAAATTGAGTTTGGATTAATTCTTCAAGAATTACAACGTATTAAAACA
CCGTATGATGCTCAATTATTATTAGTGGGTGCAGGTGGAGTAGGGTCAAT
TCCTATTACTCGTGATGATGGTTCAAGCCTGGAAGTTGAAATTTTTTATG
AAGATGATGTAGCGGGCGGGCGCTCTCGGTATGAAATTGTCAATCAAGTA
AGATTTGATAATCTACCTAAAGGATTAACAACAAAACGTATAATTGATGT
AGCTTTACTTATTAATGGTATTCCAGTAGTTCATATTGAAGAAAAAGATG
AGCATTTACAAAATCAGTGGCGAGCTTTTGAACAACTCAAAGGTTATCAT
GGTGACGGACTGTATAAAGGATTGTTTGCTTTTGTACAAGTTCAAGTTAT
CATGAGTCAACATTCGGCGCATTATTTTGCTCGTCCAAATGCATTTGAAC
AATATAATAAAACTTTTGTGTTTGGCTGGCGGGACGAAAATAATAAAGAT
ATTACGGATGCATTTGAATTTATTCATCAAGTAATGAGTATCCCTGCTCT
CCACCGCTTAGTTACAGTTAATATGATCCCGGATGCGTCAAATGATAACA
TAATGGTAATGCGCAGCTACCAAATTCAAGCAACAAGAGAAATTTTACAA
CGCATGAAAGAAATGGAAGCAAGCGGACTTGTGCAAAAAGAGGGCGGGTA
TATATGGCATACAACTGGATCAGGCAAGACAGTTACTTCCTTTAAGGTTG
CACAACTCTTAGCGTCTGCACCTAGAATTAAAAATGTACTATTTATTGTG
GATCGTATTGACTTAATAGACCAAACACTCGAAAATTTTAAAAGTTTCGC
ATATGCTCACTTTAAAAATCGCATTAAGAAGGTTAATGGAAGAGAGTTAA
AGAGAGAATTAAAGAGAAAAGGCTCCTCGCAAATTTTATTGATTTCAGTT
CAAGGATTGACAAAAGCTGTTAAAAAAGGTTTAGAAAATGACGACTGGAA
TGTCATTATTATGGATGAGGCGCACCGAAGCGCGAGTGGAGAATCTGTAA
AGTTAATCAAAAAGGCACTCAAGAAGACAACTTGGTTCGGGTTCACGGGC
ACTCCGAACTTTTACAGTGATGAAATAAATGATGTCAAAACAACTCGAGA
AATTTCAACGCATGATATATTTGGCAAACGACTACATACATATACCATTA
AAGATGCTATTGGCGACGGAAATGTCTTAGGATTTGATGTAACTTATTTC
AAACCTCATTGGGTGGTTGAACATCCACAAGAAGACTTTTCTGAAAAAGA
CTATGAAAAAGAAGTTTATCAAAGTGATGTCTATCGTCAAGAAGTCGTAA
AAGATATCCTTGATAACTGGACGAAAACATCTAGTGGGGCTTTAATTGCA
GGTAAACGTGAAGAGAATGCCTTCCAAGCGATGTTTGCAGTGTCAGGTAA
GCAAGCAGCTGTGAATTATTATAATATTTTCAAATCGAAAGGATCTCATT
TAAATGTAGCAATGACATTTTCACGTGATGAGTCTAATGAACATGGTACA
AAGGAACAAAATGAGGCGCTTAAAAATGCGATTAAAGACTATACTGAAAA
ATTCAATGTTGCAAGCATTTTAGACGCAAAGGATCCAGCTCGAGCTTACA
TGTTGGATATAACTAAGCGATTAGCACGTAAAAGACCATATAATCAAGGT
AAAGAAGAAGATAGATTAGATTTAGTAATTGTTTCAGATCAGCTTTTAAC
AGGCTTTGATTCAAAGTTCATCAACGTAATCTACATGGACAAAATGCTCA
AAGAAGGAATGCTAATACAGGCGATTTCTAGAACAAATCGAACTTTTGAC
CTTAATAGCAAACCACATGGGAAAGTACGTTTTTATCGCCAAGGTGATGA
AATGCGCGAATTTGTTGAAAATGCGCTACGTATTTATACGAGAGGTGGCA
ATGATACTCTTCAAGAAGCGGAAGAAGAGACAAAAAATCAGCTGCCTAAA
GATTTAGAAAATGACGATATTCTTGCAAAGCCCCAGAGCCACCAAATCAA
CGAACTAGAAGAAGCTATCTTAAGATTGAAAGAGCTTGCTGGCGATGATT
TTAGTCAAGTTCCACGAGGCCAAAATGATCTAAAAGAATTTGTTGGTCTT
GCTTTAACTACTCAAACCAAGATCCAGCAATTAATCCAACAGGGTTATGA
GCTAGGAAGCGAGATTGAGGAGCTAAATGAACAAGGTGAAGCGACTGGAA
GAATGGTTCGTTTGAACATTTCAAGTATTGATGAATTTGGTGCTTTACAA
GCTCGTTTATATGATGCTAAGGAGAAATTACCTCCTGAAGAGCGTCCAGA
TCTCACTGAAATAAAGATTGGAATTGAATTTTATCATCACGAGATAATTG
ACTATGATATGTTAGTTGAGCTTCTGAATACCTTTATGGACGAAAAAAAT
AAAAGTAATAAAGATGCTATCGAAAAACACATCGTACCAATGGATGAAGA
AAGTCAACATGAAATCCATGGAATCGTGGATGATATTGAAGCAGGTCATA
TCACAGAACATTTTACAACCGATTCTCTTCAGGAAACACGTAAGAAATAC
CGCACGGAACATCGAGAGCTTAAAGTTCGCCGTTGGGCAGCTAACCAAAA
AGTCAATGGTAATCGTATTGTAGAAGCTTTCGACTTATTCCTACCAGGGC
ATACGCTTATTGATAATCCAAAGCTAGCGGATATTGTGCGTGAGATTGAA
GAAGAGGAAAATATCGGATTTTTTGAAGCCTCTGATTTTGAAGAAGAGCT
AATGGCATTCTTCAATTCACTATAA
ORF2217 (SEQ ID NO:48)
GAGGACATAATTATGATTAGAGTTGTAGATTTATTTTCTGGTGCAGGGGG
TTTGACATTAGGATTTCAGAATAGAATTATAAATAACACCTTTTTACCTA
TGTCTGATTATAATATTTTATTTGCTAATGAAGTGGATAAACACGCATCT
GAAGCCTTCTCTCTAAATTTCCCACAAATTCCAATGTTAAATTGTAGTAT
AACGGAACTAACCGAGGATTATTTAGATAATCATGAGATTGAGTATTCTA
ATATTGATTTAGTAATAGGTGGCCCTCCCTGCCAATCCTTCAGTACAGTT
GGTAGGAGGCAATATGATGAAAAGGCTACGATGTATAAGGAATACAGAAG
AATGCTTTCCTTCTTACAGCCTAAAGTATTCCTTTTTGAAAATGTGACAG
GTCTTTTAACGATGAAAAATGATGAAGGGGAACCTGTACTTGAAGATATC
AGAAAAGAATTTGGCGATTTTTCTGACTTTGAAATTGATTTAACCTATGA
AATTAGAGAAACAGTTCTAAATGCAAAACATTTTGGTGTCCCTCAGAATA
GAGAGAGAGTATTTTTACTAGGGATAAGAAAGGATTTAGAAATTAAACAA
AATTGGGAATTTCCAGAACGATTATTATTTGAGGATGATTTTTTAACATT
AGAAGATGCTATAGGAGATTTACCTGCGTTGAGAAATGGTGAAAGGAAGG
ATGCTTACGATGGAGATCCGTATACTCCTTATCAAAATTTAATGAGGAAT
AATTCGACTATATTAATGGACCATTTCAATGGGTTAAACGGTGAAAAAAT
GTTGAGAATTATGGAAACAGTTATTCCGGGAGAGGGGAAAAAATATATAA
ATGAGTTAGTTGAATCTGGACAATTAGATTCAAAGTATTACCTAACATCT
GGTTATAATAATACATATAGTAAACTTTGGTGGGATAGACCTAGTTCAAC
AATTACTAATAATTTATCCACGCCATCTTCTTTCAGATGTATACATCCAA
TACAAAATAGAGCATTAACCCCTCGAGAAGGAGCTAGGATACAGTCTTTT
CCGGATACATTTCAATTTGTTGGCAGTAAGGAAAAAATTAATAGCCAAAT
TGGTAATGCTGTACCTCCTTTATTAGCGATTGTTATTGCACAAGAAATAT
ATAATTTTTTTGAAGAGAACAATTGGATATAG
ORF2247 (SEQ ID NO:49)
GGCCAGGCTCCACACGTTAGAAACCAAACAAGCCACCTTACAAAAGGAGT
GGAGCAAGTGAGCCGGATAGATATTGCGGAATTGAATGACTTTCTCCACG
GTTTGCGAAGTAGTAATGCGGAAGCCAAAGCGATGATTAGAAAAATTAAA
GAAGCAGCGATGGATTACGCCCAGGACAATCGTTTAAAAGGGGAAGCAGT
TAGTACCTCCAAACGGTATTTTTCTAGTACCTATACAAGTATTTGCCAGA
GCATCATTGAGGCACTGGATGAAAGCGAAGAGCGACTAGCGCAATATATT
CGCGAGTTCGGGAGCCAAGTGGATAGTTCGCCTTCTGCCAGAATTGATGC
AGAAATCTTACAAGAAGCGATGGCCAAGGTTAGCCAGTTACAGCGAAAAG
AAGAAGACTTGCATCGACAATTGACGGCGCCCAATACGAAGCCGGATATG
CAACAAGTCTATGCAGTGAAATCAAGAAGTGTTCATACACAATTATTGAA
GGCAATCGAAAAAGAGAACATCCTAGAAAGATATTTAGCTTTTGAACAAA
GTCATGGCCAGTTTTTTAGTGCGTTAGACGAACTCATTCAAGCGACCGGA
CGTGCGGTGCAAGAATTGTTACAACATGTGACTTTTAACGATAAAACAGG
GACTTATTCCGTGCCGAAAAGTGCGGTAAACAGCTTGTTGCTTATGAAAA
AAGCGCTGGATAACGCACGAACAGAAAATGACCAAGACCCCTTTCCGAAA
GCTTTTGAAGATTACACCGTGTTAGCTTATACTTATGTCAATGATCAAGG
CGAAACCGTGACGATGTGGTTACTTGAAAAAGATGGAAAACGAGTAGAAA
ACAAGGAATTACAAGATTTCTTAGAAAAACATGGTCAGGAATTAGACCCA
CTTCTTTATACCAACCTTTCTGGAGAAGAACTCGAACGAAAAGTGAATGA
TTCTTGGAAAGAGGGTATCAACTATTTAAATGGTCAAAAAGTATCTGGAT
TTTCTGGAGCCACACTGCGGTCATCCGCTTATGTGGCTAGTTTGAAAGAT
TGGACAGATGATGCTGGGTTGACAGATATGGCATTAGGTTTAGGGTTTGG
GATTGCAGCAGCTAGAAATAAAGCGATAATACCTAAGAAATCCAGTGGAC
TTAAATTTGACGATATAGAAAAATTGGAAAACCATTTTACAAAACATGGA
GGGGAGTTTAAAGGTGCATATTCGAATATAAATGAATATCTAAAAGGGGC
GAATAATGTAATTAAGAATGGTGAAAAAGTACAATATAATTATACATTGA
AGGATGGAACAACCGAGTTGAGAACTGGCTATGTGAAGTTTATGGGGAAC
ACTAGTAAAGGAAAAGCAAAATTTGAATTTGTTGGTACTAACTTATCTGG
AGATATTACGACCTACCATGTGAAACGCGGCGAGGATGTATATAAATTAT
TGAATGGAAACAAGCATATAAATGTGATAAATCCAATAGAATAG
ORF2291 (SEQ ID NO:50)
AAATTAACTACGAAAGAAGGAGGGGAGCTGATGAAGAAGTTTTCCATGCG
AGTGGTCCTTATTATTTCTGTGCTTTTTATTGCTTTTGGGAGCGCTAATG
TTTCTATCGCACAAGAGATGGATACAACGAATAAGCTTCCAGAAGAGGAG
CTGGGTTCTCTTTATACCTCTAATCTCATTACAGAAGAAGTCGCGCAAGA
CAAACCAGCAGAGGTGGAGCAACTAGAAGAAATTCCAACTACGGATGAGT
TGATGCAAAATCCAGATGTTCTGGAACAGCCTGTTGCTGATTCAGATGAT
CCGGATTTAACAGTGGTAAGCTCAGGTGCTTACTGGACGATTTATCGCAA
TACTGTGAACGGTGAATATAGCTTGCATATGTTTGGCAACGTGCCAAGCA
GTAGACCAACCGCTTGGAATAGCTATTTGAAAAGCATTAAACATATAGAG
ATTGAAGAAGCTACTTTGACAGGAAATTTTGCATCGTATTTTGACAATAG
CGCTTTTCCAGCACTGGAAAGCGTGAGGATTGAACAGTGTAATTTGTCTG
GAGTGACGTCTTTTGCTTCTGCATTTTATAGTTCAGGAATTGAAAAAGTT
ATTATTAGGGATAATGATTATCCGACAGCACCATCTTTACTCACTACGCA
ATCTATGTTTGGTTATGCTAACAAACTTACGGAGCTTGATGTGAGTGGTC
TTGATACGAGTGCTGTAACTAATATGCAGAACATGTTTCAGTACTGTAGA
GCACTTGAGGAACTGGATGTAAGTCATTTTGATACTAGTTCCGTTACTAC
TATGAGGGGCATGTTTCAGTACTGTGAATTACTTGATAAATTGGATGTAA
GCAACTGGGATACTAGTTCCGTTACTACTATGATGAGTGCGTTTGCTGAA
TGTAACTCACTAGAGATTTTGGATGTAAGTAATTTTGATACCAGTTCAGT
AACTGATATGACTGCCATGTTTCAAAACTGTTATGCACTGGAGAAATTGA
ATATAAGTAATTTTGATACCAGTTCAGTAACTAAAATGTATGCCATGTTT
TCAGGGCTATATGAAGTCGGAAAATTAGATGCAAGTAATTTTGATACTAG
TCTGGTTACTACTATGAATAGAATGTTTCAAAATTGCAAGTCACTTAAAG
AGCTAGATATAGGCAATTTTAATACGAGTTTAGTAACGGATATGGACCGG
ATGTTTATAAATTGTGCAGCGTTAAAATCTTTATATCTGGATAATTTTAC
TACCGCAAAGACCATGACAGATATGTTTACTGGAACCACCTCCCTAACCT
ACTTGTTTGTCAGTCACAATTTAAGTACTTTTACTGGCCTAGAAAATACA
AGCTGGTATGACGAAAAGAATTGGGTACAGTTTTCTAATCTCTCGCAACT
ACAGACATATCACCGGAAACAAAGTGAACCTACAGGCTACAGAAAAGGTG
AGTTCCTTTCCTTAACGATGGATGCGATGGGTGGAGAATTTGAAGACGCG
GAGGAACAAAAAGTACAAAGTAAAATTTCTGGGGAGTACTGGGAAGAAGT
GATTCCAGTAAAAGAAGGACATTACTTTGACGGGTGGTATCTTGATCAAA
ATTTCACTAATAAGTTTGACTTTTCATTGCCAGCTGCTGTATCTACAACT
ATCTACGCAAAATGGATAGAAAACTATACAGTGATTATTCCGGCTTCCAT
TTCTTTAAATGAAACTTCTGAATTGAAAGTAGAAGGGATTAATCGAGGAG
ATAAAAATTTGTCCGTGGGATTAAACCGAACAGCAACGTCTATTTCTGAA
AGCAACAAGCTAACCTTGTCCAATACGGCAGACGCAACAGTCCAATGTTT
GGTACCACTAAGTTGGGATGGGTCCGAAAACAATCCAGAAAATGCCATTT
TAACTCTCGCTCCAGGTTCGGAAATAACAGAAGGCGATGCAGTTATGGAA
ATAAAATCCCCAGAAAATATCCAGGCAGGAAAATATACAGGGAACGTTGT
ATTTTCGATTAAGTATGACTGA
ORF2293 (SEQ ID NO:51)
ATAAATTGGAGGTTACCTATGAAGAAGAAAAGAGTAGTGATCATATCCTT
ACTACTATTGTTAGTAAGTGTCATTGGAATCAGTAGTTATTTTCTATTCA
AGGATAAAATAAATCTGTTGGATGTAGACCATTCTGCCGTTGATTGGAAC
GGGAAAAAACAGAAGGATACAAGTGGAGAAGAAAATACAATCGCCATTCC
GGGTTTTGAAAAAGTAACGTTGTATGCAAATGAAACAACACAAGCAGTCA
ATTTCCATAATCCGGAAATTAATGATTGTTACTTCAAAATATCGCTTATT
CATCCGGATGGTTCGGTTCTATGGATATCCGATTTGATTGAACCGGGAAA
AGGTATGTATTCCATTGAATTAGAAAAAACGTTAGCGGTAGGCGAATATG
AAAATGCAGTATTAAAATATGAATGTTTTTCTTTAAACGATCAGTCACCT
TTAAACGGGTCTGAAATAAATTTAAAATTAGTTGTCGTCTAG
ORF2336 (SEQ ID NO:52)
AAAGCATACAGCAGAGGTGAAGAGTTGAAAAAAAGATGGATAATAGCACT
AGCAATTATAGTAATAACCATTTTTGGTTTAGGGGTGAAATTCTATATGG
ATGAGGAGAAATTAAACAAAGAAATGATGAATGTAGTTTATAGTGATGAA
GCAAAACAAGTATTCGAAAAAAGGCTGACAAATTTAGATCCAAATGCTTT
TACAGAAAAAGGTATAATTCATTCTTATAAAATTGAAGAAGGAAGCGTAG
AGCACAACCCAATGGGAGGTATAGAAGTCGGATTAATAATTAATAATGAT
TCAGAACTAAATGTATCATATACCTTGAGCAAGAACAATGGAGAGTTAAG
TGGTGGTGCTTCAGTTGTATCTGAAAAGCTTTCTAAACTATTAGGTCGTT
GGGAGGAGTAG
ORF2372 (SEQ ID NO:53)
AAGTTAATTTGTCTTATACTTAGTTTATCAGCTGATAATAAATCAAAAGG
GGATCGGATAATGGCAAATTTGAAAGTAATAGAAAATGAAGCAACTATTC
TAGCTTCTATACCTAAACATAATGTTGATTACAATGCTAGAGTAATGCAA
TCTGTTTCTGTTGACTACGAGCGAGATATGATTTACTGGACGCAACAGTA
CAGCGGAAAAAAAATGACTGATGCTGGAGCTGGGGAGTCATATAACATTA
CGAGGACAGATTTGAAAGGAAATTACATTGATCAAATGTGGTGTTTAAAC
GGTGGGCATGGAACAAATATTGCACTTGATTATAATTTAGAAAGTAAAAA
AATGCATATTTGGTCGGCATATAAAATTAACAATAAATGGGAAGTGGTTT
GTTACCCTTATGAAAGTAATAAAATCCTAAAGGGAACCGAGTCCAGTATT
TTCATTTCAAAAGTAGAGAGTGGCAGCTATAACCGAATTTCAGGAGATTT
GAAAAACGATATGTTGGTTTTTCATTCAGGTGGAGATCCTAAAACGTTTA
ATATTAGAATCGTGCGTGCTTCCTCTGTAAGAGAGGGAAAGCTTGAAGTA
CTTTATAAAGTAAAGGCAACTGAAGCAAATGATGCATATGTTTACCAAGG
GTGTGCGTTAGATTTTCCATATTTATATACAGCTTCTGGTACTGGTGGCG
TTGAAGAACCAAAACAATTAACTTGCGTGGATATAGTAACTGGAAAACGT
GTGTATCAAACAACATTTAAGTTCAATACAAAAGCAATGCAAACCACAGA
AAGCAATTTTGCAGAACCAGAAAATGTTTGTGTATATTATAAAAATAACC
AAAAGCATATTGTAGTAGGATATGCTTTGGGAGGAGCAGGGAATCGTATG
AATAGAGCATTTGATCTTGTAGAAAATAATAGTGAAAACATAGAAACAGA
AATTGAAAGTTTAAGAAACCTTATTATAAGTAGAAAACGTACAGAAGTTA
TTTTTGATCAAAGTACTAAAGGGGATATGACAACAAATTTTAAATTACGT
GAAACGTTAAATAATTTTGATATTATCCAAGTTGTCCTTGAATCTGGTGG
GGGGTATACAACTGCATCTCGACTCGTATCGCCAAAACTATTCGAGGCAT
CTAAATCATTTATTTTTGCTTCGAGTAATATTGGTGATGCTAGTGGAAAT
AATGTAGATATGTACGAATACGCTGCTAATTTTAATGATGACTTAACATC
TTTTAAGAATGATCGTGCAGTCAAAATCGAAGTTTCAAATAACGGAACAG
TACGGGGTAATATTACGAATATGGGTATTAAAAAAATCTATGGTATTGTT
TTGTAA
ORF2375 (SEQ ID NO:54)
AAATTAACTACGAAAGAAGGAGGGGAGCTGATGAAGAAGTTTTCCATGCG
AGTGGTCCTTATTATTTCTGTGCTTTTTATTACTTTTGGGAGCGCTAATG
TTTCTACCGCACAAGAAAGGGACACAGCAAATAAGCTTCCAGAGGTGGAA
CTAGGTTCTCTGGATACCTCTAATATCATTGCAGAAGAAGTTACGCAAGA
GAAACCAGCAGAAGTGGAGCAACTAGAAGAAATCCCAACTACGGATGAAT
TGATGCAAAATCCAAATGTTCTTGAGAAGTCTGTTGCCGATTCGGATGAT
CCGGATTTAACAGTGGTAAGCTCAGGTGCTTACTGGACGATTTATTATAA
TTCTGCGAACGGCGAATATAGCTTGCGTATGTTTGGCAACGTACCAAGCA
GTAAACCAACCGCTTGGAATAGTTATTTGAGTAACATTAAACATATAGAG
ATTGAAGAAGCTACGCTGACAGGAAGTTTTGAAGCTTATTTTAGAGATAA
TGTTTTCACGGTACTTGAAAGTGTGAGGATTGAACAGTGTAATTTGTCTG
GAGTGACGTCTTTTAGATCCGCATTTGAAGGTCAATTAGCGCTTGAAAAA
GTGATCATCAGAGATAATTATTATCCGACAACGCCAACTTTACGCACTAC
GGAAAATATGTTTCTGGGATGTTACAACCTTAGGGAGTTTGATGTGAGTG
GCCTTGATACTAGAGCAGTAACTAATATGAGGGCCATGTTTCAAAATTGT
ACTAGCCTTGAAGAGTTAGATGTAAGTAATTTTGATACAAGTTCAGTTAC
AACTATGCAGAATATGTTTCAAAATTGTACTAGCCTTGAAGAGCTAGATG
TAAGTAACTTTGACACCCGTTCAGTAACTTCTATGGCTTACATGTTTTAT
AGGAGTGCCGGTCTTAAAGAATTAGATGTAAGTAATTTTAATACTAGTTC
AGTAACTGCAATACAGGGCATGTTTTATGATTGTTCTAGCCTTGAAGAGC
TAGATGTAAGTAATTTTGATACCCGTTCAGTGACTTCTATGACCTTCATG
TTTTTGAATTGTGCTAATCTTGAAGAATTAGATGTAAGTAATTTTAATAC
CCGTTCAGTAACTTCTATGACTTACATGTTTGGAGGTTGTACCAGTCTTA
AAGAATTAGATCTAAGTAATTTTAATACGAGTTCAGTTACAGCTATGTAT
AATATGTTTCAAAATAGTACTAGCCTTGAAGAGCTTGATTTAAGTAATTT
TAATACGAGTTCAGTAACTAATATGTCTTACATGTTTCAAAATTGTACAG
CGTTAAAATCTTTATATCTAGATAATTTCACTACTCCAAAGACTATGACA
GATATGTTTATTGGAACCACCTCCTTAACCTACTTGTTTGTCAGTCACAA
TTTTAAGGATTTTAATGGGGTAGAAAATACAAACTGGTTTGACGAAAAGA
ATGGGGTACAGTTTTCGAATCTCTCGCAACTACAGACATATCATCAACAA
CAAAGGGAACCTACTGGCTACAGAAAAGGTGTGTTCCTTTCCTTAACGAT
GGATGCGATGGGTGGGCAGTTTGAAGACGCGGAGGAACAAAAGGTACAAA
ATAAAGTTTCTGGGGAATACTGGGAAGAAGTGGTTCCTGTAAAAGAAGGA
CATTATTTTGATGGGTGGTATCTTGATCGAAACTTCACTAATAAGTTTGA
TTTTTCTTTGCCTGTTGCTGTATCTACAACTATCTACGCTAAATGGGTAG
AAAATTATACGGTGGTTATTCCAGCTTCTATTTCCTTAAATGAAGCTACT
GAATTGAAAGTAGAAGGGATTAATCGAGGGAGTAAAACTTTATCCGTAGG
CTTGAATTATGAGGAAACAACCATTTCTGAGAGCAACAAGCTAACCTTGG
CCAATACGGCAGACACAACAGTCCAATGTTTGGCACCACTGAGTTGGGAC
GGGTCAGAAACTAATCCAGAAAAAGTGATTTTAACCCTTGCCCCAGGTTC
GGAAATAACAGAAGGCGATGCAGTCATGGCTATTGAAGCGCCAGAAAACA
TCCAGGCAGGAACGTATACCGGGAATCTAGTATTTTCGATTAATTATGAA
TAA
ORF2440 (SEQ ID NO:55)
ATAATATTCCACATCCAATTGCATAACATCTTTTTTTTAATCACATTATT
TCCAGGTATGCTTCTTTTATTGACGAAATGGATTCCAGTTCTATCTAGAA
AGAGTACTTTTTTTCAATATTTACTTTGTTTATTCTTGATCACAATTATG
AATAGTTTGTTTTTCCGTCAACAGTTTGTGGTAGTTTTATCGCTGATATG
TATTTTATTCTTACCATTTATTCTGTTTTTTGTAGAATATATATTTGTTG
AGAGACAATGGAAAAAGTTGCTTACTATTTATAAAAAAAATAAAATTATT
ATCCAATCTATTGTATGGTTTCCTGTTTTAGAAGAAATAATTTTCCGTTT
TTTTATTTATCAATACTGTGAGTTATTTGATTTCAGTAATATCCAGTATA
TATTACTAGCCACCTTTTCATTTGTGATTGCACATATTTTTTATCAAGGA
GTGTCTTCAATTGTCAAAATACTATTTTCTTTTATATTAAGTATATTATT
TTTATTAACACTAAATATATTTTTGACAATAATAATTCACTGTATTTTCA
ACTTTTTAGTTTATATAGTTCGTACTAGTAAATATGAGAACCACCGTAAT
TGGTAA
ORF2462 (SEQ ID NO:56)
GGGGTTGTAGTGAGAGTGGAAAAGCCATTTTTAACAGTTGTGGTACCTTG
TTATAATGAAGAAGAAGTTCTGAGCGAGAGTGTTACACAACTAACAAATA
TTATAGAAAAATTAGTGATGAGTGAATCAATCAGTGACAAAAGTCAAATT
ATGTTTGTAGATGATGGAAGTAAAGATAGAACTTGGGAACTTATACAGCA
ATACTCAGAATCAAATGAACATGTATCTGGACTAAAATTAAGTCGGAATT
ATGGACATCAAGGAGCTTTACTTGCTGGCTTAACAGAAGCACATGCATAT
TCAGATTGTGTCGTGTCTATTGATGCTGATTTACAAGATGATGTGAATGC
GATTATTGAGTTTATAGAAAAATATCATGAAGGTTACGATGTCGTTTATG
GCGTACGAGATAAACGTGACACTGACACTTACTTCAAAAGAAATTCAGCG
TTAGCTTTCTATAGAATTATGAGCAAACTAGGAGTTAATATGGTTCCAAA
TCATGCGGACTATCGACTGCTTAGCAAGCGTGCATTAACAGAATTCTTAC
GTTATAAAGAAGAAAACATGTTTATTCGAGGCATTGTACCATTATTAGGT
TTCAAATCTACGAAAGTATTTTACAATCGAAATGAGAGATTTGCAGGAGA
ATCAAAATATCCACTTAAGAAAATGGTTTTATTTGCTGTGGATGGAATTA
CATCTTTCAGTGTTGCACCCATTCGATTATTATTAGTCCTTGGATCTGTT
ATTTTTATGATAGGCGTAGTAATGGGGATTTATGCCATTGTTCAAAAAAT
CATTGGTGCTGTTGTACCTGGTTGGACATCTTTAATTGTTTCCTTGTGGT
TAATTGGCGGAATTCAATTAATTGGTATTGGTGTTCTAGGTGAATACATT
GGAAAAATCTTTAAACAAGTAAAAGAACGACCTCGTTTTACTATTGAAGA
GAATGTTTTTGAAACAAAATGCAAAGAAAATAAAATCAGCGAGAGATAG
ORF2541 (SEQ ID NO:57)
ATCAAGTTGCTAAAATACAAGATACAAGGAGTGCTATATTTTTTGAAAAA
ATTTGTTTTAATTTTTATTTGTTTTCTCACGCTAACAACCATTGTCCCTT
GGAATACGCTCGAAACGAAGGCGGCATCCACTTCTTGGCTTGAACAAGAA
TTAGATGGTAATGAAGCCTTTATCACTGAAACAGAAAGAGTCTTGTCAAA
AAATCGTGAAGATATTACACTCGCAGATTTAGAAACAATACAAGAATTAG
ATATTTATGGTGATGCTTCATCAATACCCGACAAAATTTCTGATTATAAG
AATCTCAATACTTTACTTGCCCTTAATGGAACGATTTCAGAAATTCCAAC
TAGTATCACTAAACTAACAAAACTAACAAGAATCAACGTAGACAATAATA
ATTTTCAAGAATTTCCGATGATTCTATTACAGATGCCTTCTCTAAGTTCT
ATTGAGATAAATAGAAATAAAATTAAAGAGATTCCTTCAGAAATAACCAC
ATTATCCCCTCATTTAGGATCGCTGGATGTACGCTATAATGAGCTAATTA
CGCTTCCGGATAATATCTTCACTACTGAGTGGGAAAGTAAGCTCTCATTA
CTAACTACAGGAAATCAATTAGTTTCTGACATTCCTGCTGATTGGTTAGA
TAATTTTAATCAAGCAGATAACATGCTCGAATTTTACAACAATCCACCAA
ATGATTATCATCAAAAACAAGACCAGCTAACGTATAGTGGAGCAAGAATT
GAAGTCCCATTAAATACAGATCTAAAAACATTAACTCCTGATAAAACAAA
ACTGGGCTTAAAATCAGGCAGAACGTTATTTGAACAACATGAATTTATGT
ATTATGATGACGGGACCTCTAATAACATTCTAACTAACGGTGTTGCTACT
GCCACAGGAAATGGCTATATAACAATCAAAAGTACTTTATCAACCAATTC
CAACCCTTTTGCAAAAGTACGAGTTCCTATCACAGTAACGCCCCCAGTAA
AAGGAGGAGATGTTACCGTTCAATATAAAGACACAACTGGAGTAGTGTTA
GCAGACTCCATTACTTTATCCGGAAATGTGGGCGAAAATTATACGACAAC
AGCTAAAACAATTGACGGCTATTCTTTAACAACTACACCAACCAATGCTA
ATGGAACTTTTTCAACAAACCCACAAACTGTCACATATACTTATAAAAAA
GACCCTATAGCGCAACCAGTTACTGTTAATTATATTGATACAGATGGAAA
AACAATTGCTCCCACTGAAACATTAAGCGGAAATGTGGGCGAAAATTATA
CGACAACTGCTAAAACAATTGACGGCTATTCTTTAACAACTACACCTGCC
AATGCTAATGGAACTTTTTCAACAAACCCACAAACTGTCACATATACTTA
CACAAAAGATCCTATAGCGCAACCAGTTACTGTTAATTATATCGATACAG
ATGGAAAAACAATTGCTCCTTCTGAAACCTTAACCGGAAATATTAGTGAA
AACTATACGACAACAGCTAAAACAATTGACGGCTATTCTTTAACAACTAC
ACCTGCCAATGCCAAGGGGACTTTTTCGACAGAACCTCAAATAATTAATT
ATATCTATGCGAAAAACGCTGAGACAGCCCAACCTATTACCGTTAATTAT
AGAAACTCTACAGGTCAAAAAATCGCGAAAAGTGAGGTTTTAACTGGTAA
TATCGGAGAATCTTACTCAACTCAACCGAAAACAATTGCTGGTTACACAC
TCACAACAATACCTCCAAATGCAAAAGGAACCTTTACAACAAATGCGCAA
ACAGTAACTTATATGTATACGCCGATTGCTATTTCCGCCTTACCAGTGAC
AGTGAACTATTTAGATGAAAATGGTAAAGAAATTGCTGACTCTGTAGTTC
TAAATGGTCGTGTGGGAGAGGCTTATAATACACTAGCTAAAGAAATAGAT
GGCTACACGTTGATAAAAACTCCTACAAATTCTAATGGTGTGTTTTCGGC
GGAAGCACAATCAATCGATTATATCTACCGTAAAAATAAACCAGTAGTAA
TAGTAAATCCACCAATCAAAAACCCGATTATTAAAATAGATTCTAGTACC
AACGTAATAAAAGAAGAAACGTCTGTCTTACCAAAAACTGGCGATTCCAA
TCCATACAATGACTTTTTGGCAGGAATTCTACTATTATCTTCAGCGATGT
TTCTTTGGAAAAATCATAAATAA
ORF2568 (SEQ ID NO:58)
AATTGTAAAGTTGAAGGGGCAAAAAAAATGAAATTAGGTAAAGTAGGAGC
CTTACTTCTCGGTACGGCTTTAGTTTTCCAGGCACCATTCCAAGTTTTCG
CAACGAGTGTAAATGGAGAAGAACAGGTGAAAAAAGAAGCTGCTGCGCCA
AAAATATCCGTTAACAAGCAAACCTTAGCTGCTACCAGTGATTTAGAACA
AACGGTAGGGGAAGCAATTACCAAAACGATACATCTTCAAGATCAAGCAT
TACCAAACAATGAATGGGGTAGCTATATTTCTGACGTGAAAGTAACATTA
TCCAATATGGACGGAATTAGTTATGACGTGAAATACGGACCGCTAAGTGA
AGACGGTACGCACTATCAATATGCAGACATAACACTTTCAGGAGCACCGA
CAAAAGCTGGAACAGGAAGCATTAGCCTTGAATATTACGACGGGGCAGGT
AACGGTGGAGTTTACAGTTATTCTGTAAACACAAAATCTAAAACAACTAT
CCAATATGTAGATGGTGATGGAAATAAGTTAGCAGAGGATACAGTGAAAA
CGGGTGATTTAAATACAGCCTATACCTCTGAACCAAAAACGATTGATGGA
TATGAAGTAGACGAAACAAAACTTCCTAGCAATCAAAATGGTCAGTTTGC
TGAAACAAATCAGACAGTTACGTACATCTACTCAAAAACGAAAAGTGAAG
TAAATAAAGGAACGGTCGGTATTTCATTCTATACTCCTGATGGTAGCCGC
CGGGAACTTAAGTCCTCATTAGACTTAAGTTATGCCTACCCAGATGGTGT
CCCAACAGACACAGTAACATTCGGCGATTTAGCAAAAAATACGACTTACA
AAGATTTAAGAAATACGCCTGATGCGCCTACTCTTTTATGGAATGATTTG
TTACATTACATGGTAGACTACATGAACGGCGATATAGATGCCGCTCAATT
TGAAGAGGCAGTTGGGCTAACACCAGCGAATTTTGATTTAGATTTTATTT
CACGAAATTTCGAAGGATATAAATTTGATGAAGCAATGTATCAAGAAAAT
CTAAGTAAATTAGTCACTTTTGAGCAAGATGGCGATCAAGTTGATTTGCA
AGTACCATTTAAGAAAGTAGAAGTTGGAGCGGATATTACTGTAAAATACG
TTGACACAGCAGGAAACGAATTAGCATCTGTAGAAACGTTGAGTGGTGAT
GTGGATGATAACTATACTTCAGAAGCAAAAACAATTGATGGTTGGACTTT
AAAGGAAACACCTAATAATGCAACAGGAGTCTTTAGTAAAGAGGCACAAA
CGGTTACGTATGTTTATGAAAAAAATGATGATACAGATGTCACACCGGCT
CCTGACAATTCAGCAGATACAAATGATGACAGTAACTCATCAGATAATAC
TGCGACAACAGATGATAATAGTAGTAGCGCAGTGAAAGAAAAGGCGCAAA
CACAAGCAAGTAGTCCAAAGGCAGGAAAAGTAACCACACAAGAGAGCACT
TCTAACAAAGTAACAGCACAATCAAAAGAATCACTTCCTAAAACGGGAGA
TAACGTGTTGGAAAGTAGCTTGTTAGTGGGCTTGGGTATGCTTTTGTTAG
GGGGATTATTTGTATTCCTTCGTAAAACTAGAAAAGTAAAATAA
ORF2646 (SEQ ID NO:59)
GGATGTGGTTTTTTAGTGAAGCGAATACTCATTAGATCGGGTATGCGACC
GACCAATGTCTACACGCCGGAAGACATCTATTTAAAGGATCGAACCGGGT
TTAACAATGGTAATCTTGCGTATCAATATAGCATTTATCGAGCTTTGTGG
AATGATGATGTAGAAATACATGCTGACGGTCTTTCATCTAACCCCAATCT
CGCAGAAAAAATCAATGAAAATTACGACCTCTATGTTATGCCTTTAGCTG
ACGCATTTCGAGACGACTTTAGGCCAGTACTCCGCAATTACACCCAACTC
ATTCGCAAATTAAAAATCCCAGTCATCGTAACAGGCGTCGGGTTAAGAGC
GAATTACGAACCACAACTAGACGAAGGTTTTTCTTTTGATGAAGATGTTA
CTAACTTTGTTAAAGCAGTCCTAGAAAAATCAGCTCAAATTGGGGTTCGA
GGACAAATTACTGCAGATTATTTAAAAAAACTAGGATTTAACGCAGAACT
TGACTTCCGAGTAATTGGTTGTCCGTCCCTTTATACATTCGGACGAGAAA
TAAAAATAAGAGATTTTCACTTAACAGATCAATCCACCATCGCTATTAAT
GCATCCCCAACTTCTTCAGAAATAGCTATCAATTTCCTTAACAACATGAT
TACTACATATAAAGACTATCACTTTATCCCTCAACACCTAGACGAATTCC
ATTTAATGTATGCCGGCGGTCCAGATATCTCTAGTGATATCAATGGCTAC
CCAACAAATATCCAACATAAGTATTATCAAGAAGGTCGAGTAAAATATTT
CACTAGCATGCCTAGTTGGTTCGATTTTGTGAAGAAAGTTGATTTTAGCA
TTGGCTCTCGCCTACATGGAAACGTCATTCCAACAATAGTCGGAACACCT
AATATTTCTTTCGTACAAGATGCCAGAATGCGTGAACTAGCTTCCTACCA
TGCGCTTCCTCACGTAACCATCGATGAATTAGAAAAAACCAACAACCTCC
AAGAATTACTAACTAAAGTGGACCTGAAATCAGCCGAAAAAGTCCAAGCA
AGAAATTTTGATAATTATATTGATTTCTTAGATACTAATGGTCTCAATCA
TATCTATAAATACGATAAAAACAGAAAATCAGCTCCCATGGATGAACTTA
TAAACTCCATTACTTTCCCAACAAGTCCAGACCCCATTTCCATATTAAAC
CCTCAAGAAATGCTAAACCGAGTAAAAATCTCCACCAATTTATTAAAAGA
AAGACATGATTTTAGCACAAGATACCGTGTCAATCTCGTAAATAATCAAC
TTACCCAACTGAAAAAAACAACTTCTGAACAAAACAAAAAATACCAACAA
AAAATTACTGAAACACAAGAAAAAAATCAGCAATTAGAAAAACAACTGAC
AGATACTAAACAAAAATTACAGTTGACGATTAATAAAAATCACGAACTAA
CTAATAAAATCAGACATTATCAAGGAACATTAAACCGAAAATCTGTAAAA
ACTACTTTAAAAGTAGCGAACTCTCTAGCAAATTTAAAAAAGAAAGTCTC
CAGAAGCTAA
ORF2666 (SEQ ID NO:60)
ATGGAAATGAAAAAACTTATTAAATGCACTGCACCAATCTTTATTGCTAC
GCTCATTATTTTGCCTGTTTCTCCGTCTATTTCAGCATTGGCGAATGAGA
CCTCAGAGATTAGCGAAGGGATTAATGCAACAGAAAAAACAATTGAAACA
GAAGAGCTTGTCGTAGAAGAAATTAGTAATGATGAATATCAAGTACTGGA
TAAAGAGACAAATGAAACAACTTCAATTGAATTTTCAAAAAATCATATGC
AATCTACTATTACAAATCCAGATGGCACTATAGACACAATGATTAAAAAG
GATAACTTAATTTACCTAAATGGTGAAGTTATAGGTGAAGAGGTAAAAGA
AGAATCGAGACAATTATTAAAAGCCTCTGCTTATAAATATGTTACGACGT
TTAAAACAAAAATGTCACTAAAGAAAACAAGTGCATCTATTGCAGTGTCA
TTAGCTGGTTTGCTTGGAGGACCCGTTGGAGTATTTAGTACAGTAGCAGG
TATGCTACTAACATTGAAATCTTATGCCCCGAGTAAAGAGGTTTATATAA
AAATTAAACAATATTATAATAGTTACTCAAGGGAAATAAAAAATGATTAT
TCTATTTATAAAAAATCTAACTATACTGGACTGCTTAAGACATTTACACA
TAAATATCGTCCGTACGGTTGA
ORF2758 (SEQ ID NO:61)
CATTGGCTAATGGTTATTGTATTATTATATAATGCCAAAAGAATCGAAAC
TGTTTTTTTGTGTTTATTATTAAGCGAAAATGCTGTCGGAAATCAAGCAT
TTGCAATCGATTTTGGCAAAATAGAAGACATAGATGTGGCTTTCAAAAAT
ACAAAACGGTATAAACTTTTTCCGTCTTGTACTGACACTTTTAAGGAAAA
TTATTTTCTAGGGAGTTTCAGCAATATGAAATCCGCTTTCTTTTTGTCTA
AATAG
ORF2759 (SEQ ID NO:62)
TTGCAAATTAAAAATAAAGCTATGTTAATTACTTATTCTGATAGTTTAGG
GAAAAATATGGAAGAATTATCCAAGGTGATGGAAACTTATTTTGAAGATG
CTGTTGGCGGGATTCACTTATTGCCGTTCTTTCCATCCACTGGAGATCGA
GGGTTTGCGCCAAGTGATTACACAACAGTAGATAGCGACCTAGGTTCTTG
GGAAATAATCGAGAAATTAGGCGAAAAGTATTATTTAATGTTTGATTTTA
TGATTAATCACATTTCTCGCGAATCACTCTTCTTTCAAGATTTTAAAAAA
GAGCATTTAAACTCAAAGTATAAAGATATGTTTATTCGTATCAATGATTT
CTTTCCTCCGGGTAGACCAAATGAAAAAGACTTAGATTTAATTTATAAGA
GAAAAGATAAGGCGCCTTTTCAAGAAGTTGAATTTGCTGATGGGGAAACG
GAATTAGTTTGGAATACTTTTGGGGAAGAACAAATTGATTTAGATGTGAC
AGCCGAAGTTACAAAAGAATTTATTCGTCAAACGATAAAAAATATGGCTG
CACATGGTTGTTCTATTTTGCGTCTTGATGCCTTTGCTTATGCAATTAAA
AAATTAGATACAAATGATTTTTTTGTAGAACCGGAAATTTGGGATTTACT
GGATGAAGTGAAAGCAGAAGCGGCCAAATATGACATGGAATTATTACCAG
AAATTCATGAACATTATTCCATCCAAATGAAAATCGCGAACCATGATTAT
TATATCTATGATTTTGCGTTACCCATGGTGATGCTGTACTCGTTATATAG
TGGCCGAGTGGAACGTTTAGCTAACTGGTTAGAAATGAGTCCGATGAAGC
AATTTACTACTTTAGATACGCATGATGGCATTGGCGTTGTGGATGCACGC
GATTTATTAACAGATGAGGAACTCGATTATACTTCAGCAGAATTATACAA
AATAGGAGCTAATGTTAAAAAGATATATTCCTCTGAAAAATATAATAACC
TGGATATTTATCAAATTAATAGTACCTATTATAGTGCTTTGGGTGACGAT
GATAAAAGTTACTTGTTAGCGAGAGTAATTCAATGTTTTGCGCCGGGGAT
TCCGCAAATTTATTATGTTGGTTTACTTGCTGGCAAAAATGATATTGACC
TTTTAGAGGAAACAAAAGAAGGGCGTAATATTAATCGTCATTACTACACA
ATAGATGAAATTAAAAATGAAGTAAAAAGACCAGTTGTTAAAGCGTTGTG
CAATTTACTGAGGTTTAGAAATACTTCTGAAGCGTTTGATTTGGAAGGAA
GTATAGAAATTGAGACACCTAGCTCAAATGAAATCGTTATTATTCGCAAA
AACAAAACAAATAAAATTACAGCGACATTAAAAGCAAATTTAAGTACTAA
AACATTCCAAATCAGCGAAAATGAAAGAAATATTTTAATTTAA
ORF2763 (SEQ ID NO:63)
TTGAAGAAGATTTTACATATTGTTATCGCACTTAGTGTAATTTGTTCGAT
AATTTTAGTGCCAGTAGATTGGTCCGCAAAAGCAGGCGGTAATAATAAGC
GAGATGATCTTATTAAATCTGTCAGTTTTTATAATTCAAATGGACAAAAT
GTAACGGCGAAGGAAAACTATAATGAAAAACTAAATTATTTTTTAGAAGT
AGCTTTTGGAGGGAATTCTTTCCAAAAAGGGGACTATTTTAATATTACTT
TATCATCTGATGCCTTACTCTATACAGAGAAAGCCTATGATTTGAAAGTG
GATATTGACCCAACAGCGGTAACTAATGAACAAGTAGTTGGTAAAGTTAC
TGTAGAAAAAATTAATGGATCACCAACATTGCACTTTGTTTTTACAGAAG
ATTCAGAGTCTTTTTTTATAAACAGCTTTGATGCGAGTTTTAAAATACAG
GTGATGCCAGCGCATGGCGATAAAAACGTTATTAATCTTTCCTATAGTGG
CGCTGCAAAAAACTTTAAAAACATTGGGACGAGCAGTGTGGAATGGAACG
TAAATATGGCAGATGACTGGCCGCCAGTTGGAATTAGCGATTTTTCTAAA
ATAAGTGGCGATTTATACCATGCGATTTTAGTGTATGAAAAGCCGAGTAG
TAAAGTGAATTACGAAACAGAAATTTTGGTATCGTATCCGCTTTTTGAAA
AACGAATTCCACTTGGAAATGTGCAAAATATTAAAATTGAAGTTTGGGAT
GAAGCGAAGGAGATTTATAGAGTTGGTGTAGCCGGGGTGGATTACGGAAC
GATTACATACGATGTTGGAACTCCATTTGTAGGCGGTCCTATTTTCCAAA
TGAATTGTACTATTCCGTTCAAAGGGATTTCTACTAAAACTCGTGTTAGT
TTTGATATTGATACGAATGTGGACGGGAAACCTGGAACAACAGATCCGTA
CTTAGTTAGTCTTTCTTCCGTATCATCAGCTACAAAGTCGCTGGAATTCT
ATCCAGTGAATAACGCTGATACGAAAATGACAGCTGCTTTTTTTGGGAAA
GTAACGACGCGCTTTGAGGATGAGTTAGGCAATCCAGTTACTTTTGATAA
TTATAGTACTGTAACAACACCAGGGAAAGTCAATCAAGATGGTAAATTTG
AAATAGCGGAACCTTTTTTACACAATTCCGTCCAAAACGTGGATAAACAC
GCCTATGATACCCTTTTGGATACTAATAAATATAAATTATTAGAGGTAAC
TTCTCCTAATAAACTATCTGAAACAGCGGATAATTTAAGTATTCAAATAA
AGCGTGGTTATCAGAATGATGTTTTATACAAAATAAAAGCTCTCCAAAAA
CCTGTCATTAGCGCGCTTCCTGAAATCGAATATAGTAAAACAGTGAATAG
GACCATGGAAGAGTTTTTGGAAGATGTGGAAGCAAAAACAGATATTCCAG
CTGATATAGACTGTGATTTAACGAATGTGAAATGGGGTGTTCCGGGGGAT
TACCTAGTTCTTATAACCGCGGTGAATGAGGATAATCAAGCAGCGGATCC
TGTTCCTGTAACGATTAAAATTTCCAAAAATCCCGCACCAGTCATAACGG
TAGACCCGGAAATAATCTACGATAAAACAGTGACGAAAGAGGAGAGCACA
TTACTAAATGAAGTAAATGCGCGAACAAATGACGGTTCGACGATAACTTC
TAACATTAACGACAAAGTAAAATGGGGCGTGCCAGGCGATTACGAGGTTA
CTTTGAACGCGGTAAATGAAGACGGCGTGGCAGCGGAATCGAAAACATTT
ATCGTCCGCATTTTAAAAAGCCCAGCACCAATAATCACGGTAGATCCTGA
AATAACCTATCCGAAAACAATAACTAAAACGGAAGCTGAACTCCTTCAAG
AAGTAAATGCGCAAACAAATGATGGTTCACCGCTTGTTTCTGATATGAAT
GACAAAGTAAAATGGGGCGTGCCAGGCGATTATGAAGTTACGTTAAACGC
GATAAATGAAGACGGCGTAGCAGCGGAAGCGAAAACATTTATCGTCCGCA
TTTTAAAAAGTCCAGCACCAATAATCACGGTAGATCCTGAAATAACGTAT
GATTCTTCTGTAATAAAAGACGAAAGAGAGCTATTAAAAGATGTCCATGC
TCGTTCAAGTGACGGTTCGGTAATAACTTCTGATAGTCAGGTAAAAGTAA
AATGGAAAAAAACAGGAAGCTATACCGTTACTTTAAATGCAGTAAATGAA
GATGGCATTTCTGCTAATTCTGTGCAATTCACTGTGCATATAGTAGACGC
TAAAGCAATACCAGTAGTAATTGAAGAAAAGCCTGAATCCACTCCAAAAC
CAGATACTAAAGAAAAAGTAGTTATTAAGAAAGAAAAACTACCAAAAACA
GGAGATACGAATACAAAAACTATATTGAGTGGGATTTTTTGTTTAGGCGC
TTGGTATTTGCTTAGAAGAAAATAG
ORF2777 (SEQ ID NO:64)
CGAAACGGAGTACATAAAATGAAAAATCAGTTTACCTACTTAATTAACAA
TAAAACTACTCAAGGGATGTTCATTTTAATCTTATTTATACCTTGTATTG
AAATCGTTCAATTATACATAATGTTAAAGCCAGATGCAGTTAATATTCAT
CCTGCTTTTGCATTTTTTCTTGCAGGTTCAAGCCGAGGTCATATCACTCA
AATATTATTACTTTGGTTCTTAACTGTTTTAAGTCTGTTACTCGGAGCAG
ACTCGCCCATTCAGGAATATCAGACAGGAGTTCGTAATATTATTATTAAT
AAAATAGGGAAAAAAGCTTATATTCTTCAAAAATTAGCTGTAAGTTTTAT
TCTTTGTTTTATCACCATGCTTACAACTTTATTATTAAATTTTATTTTAG
TTTCTATTGTATTCTTAGGTGGCACTTATAAAATGGGGTTAAATGGATTA
GGATCGCTTAATACTCTATTTGATTTTAGCATTCAAAACCCCTACTTAGC
AGATTTTGGTTTTGGATTTATGGCTTGCTTAATGGCCGGAATGGCTGGAC
TTATCGCTACTAGCTCCAGTCTGTTCTTTTTGAATAAAAAATTTGCGTAT
CCAGCTGCATTTTTTATTTGGTTTCTGATGATACTTCCAAACAATTCAAT
TATGTTTATTTTCCAACCTTTTACAGAATATGGCTTTGAAATAATTTTGC
CTATTTTTCTAGTTTTCAGTTTAATAGTTTTGATAATTGTTGGTGTCCTT
TACTTATATGAGGTGAAATATGTTAAAGAGTAG
ORF2778 (SEQ ID NO:65)
AATATGTTAAAGAGTAGATATTTTCTTCTAATTTTTTCCATGTTCCTCTT
TTTTTCTGTTTTTTGGTTCACACAAAACATGGAGTACCTAAGTTTCCCTA
AAAACAGAGAACTTGTTCTCATTATGAATGGTAGTTTGTATGGGTATATC
TCAATCAAATCCCTTTGCTTGATGCTTGTTTTCCCGTATCTTATCTTTTT
ACTACTATTTTCAAAAAAAGAACAAATTGTTGCTTTAGCTAGAGAAAAAA
ACCGTTTACGATTCTATCATAAAATATTGAAAGATACAGTTATTGCAACA
GTACTATTTGTAGGTTTATATCTTAGCGTAAATTTGCTATATTCTTTCAT
TTTTCTAAGTAATAAATTGCTCACTGCTACTCATTTTTATAGTGGAATTT
TTTTTACTTCTTGCTACTTGTTCTCTTTTATTTAG
ORF2779 (SEQ ID NO:66)
TGGAATTTTTTTTACTTCTTGCTACTTGTTCTCTTTTATTTAGCTATTGG
TTTTCTTTTCAGAATTATATATGACTTAACTACTTCAACTGGCCAAGCGC
TTATCTTTGGAGCTTTCGTGATATGCATTGTTTATCTTATTGATTGGATA
ATTTTAGAAGGTATATACTGGACACCTCTTCACAATTTAAATTTCTTTGA
TCTTTGGTTACAAAATGGTTCTATTAGTTCTGATATACCGTTCATTCTAA
TTCCAAACGCCGCGGTAGCATTTATCCTATACTTGATTAGTTCTAATACC
TTTATAAAAAAGGATTTTTACTAG
ORF2780 (SEQ ID NO:67)
AAAAGGATTTTTACTAGTATGAAGAATAATAAACTTATCATTTTAGTAGT
TATTTGTTTGTTTCTACAAGCAATATTATTCATGGCCTTTGACTTCCCGT
TCAAAACATTACCAATACTAGATGGATTTCCAGTAGGATTAGCCACACCG
GTAGTAACCAGATTATTGCTTTACTGGTATCTCCCGATTATTGCTTTTAG
TTTCTATATTTCAGGTAATTTAAAAGATTTATTATCTAGTTATGGTTTTT
TACAAATTTCTCGAAACTATAAAAAAGAATATTGGCTAATGAAGCAATTT
TTAAAACTGTTAATAAAAGTTATTCTTTTTACCAGTTTACAACTCGCTTT
AATTTTTATATTCACGCCCTATTCTATATATTCTACTGCTCCTTTTTTAT
ACTTAATACTTGGATATTCCATTATGCTATTTACTATTTTTAGTTTGCAA
TACCTTTTAGAGCTATTTATAGATGCGCAAAAAGCTTTATTACTTATAAA
TGGATATGTAATTATTTCTATTCTTATGGCAGATTTAATCTATCAAAACA
CAACAGTTACTTGGCCTTATTATTTATTACTTCCTAATTATGGTATGGGC
TTTAGAACTGGATTAATTCCGTTTACTAATACATCTACTTTACTAATCTC
AGTACCTACCAGTTTAATTATTTTGCTAGTAGTTTTACTTTGCGTGTTTA
TAGTCGCTATTAAAAAATTTAAAACAACAGACATTTTATGA
ORF2815 (SEQ ID NO:68)
AGAGAACAATTGGATATAGAAGATTTTATTGAAATAACAAGAGGTGAAAA
CATGAACGAAAAACAAAATGAAATCCCATTTGATTTTTCTTATTATGCTT
TACATTTATTAGGTAAAGGGCTTTATAAAAATAGATGGAGTGCAATATCT
GAATTAATAGCTAACGGAATAGATGCTCGTGCAACAAAAATAAGTTTATA
TATGAATCTAATCGATAAAGAGAAAGCAGTTATAGAAATTTTTGACAATG
GGACAGGTATGGACTATGACGATCTTGTTTCGAAGTATGTACATATTGGA
AGAAATAAGCGTGATGAAGAATTGGATGATGTTGAAAGAAATGCTCTCAT
GGGGCGCAAAGGGATAGGGAAATTAGCAGCACTAAATCTTTCACAAAAAT
ATTATTTAATTTCTAAAACAAGAAATGAATCTTCCCTATGGTGTCTGGAT
GCGACTGAAGTAAATAAATCTGACACTCCGAAGTTAAAAAGAGTAGAAAG
CAAATCAGTAGCATTGGAAAGCATAGAACATTGGAAAGAAAATTCAACTG
GTACCATGATAAAATTAACCAATGTTGATATGACTGGCTTTGGAATTCAG
AGTATGGAAGGATTAAAATTAAAACTTTCTGATTTCTACTTGTTAAATCA
AATGAGCTGCGAAATAGAAGTAGCTTACATTACTACAAAAGAAGAAAAAA
ATAACATCAAGTTTAAAAAAGTGGAAAAGAAGGTAGCATTTAAGAATTTT
TATGGTTTTTTTGAAAATATGGAAAATGATAAATATAAAGCTTCCCTAGC
GGATACAGTTAGATTTCCATCGGTATATGAAACAATTACTGAAAAACCAA
GAAAGGTTCTTTACTTTGATAAACAAAATTTTCCGGAAATTAAAGGAAAG
AGAAGATTTAAAAATAAAAATGGAACTTTAAGTGAGAAAGAATACGAATT
TGAGTTGAAGGGATGGATAGGGATCCATACATCAACAAAAAAAGATGATG
CAGAAAGGAATGATATTACTTTCTTTAGAAATAATACCTATACTCCTAAT
AAATTGAGACTTTATATACGGGATAAATTGATTGTTGAAGATTTTATGGC
CCAGTATATTCGAAGTACTCAAGCAACTTCTGGATATATTGAAGGTGAAA
TTAGTTTTGATATTCTTGATGTCAATGACTTAGAAGATATTACAACATCA
GATAGACAAGGATTCACTCATGAGGATGATAGAGTTAAGTTGCTAATTGA
TATTCTAAAGCCGATAGTAAATTTGCTTATCAGAGAGCGAAATAAAATGG
GGGGACAAATAAGAAAAGAAGAAGAAGAGTATCGTGAACAAGAAAGAGAA
GAGATTAGGAAACAAAAAGATGTCGAAGCAATAAAAAGAAAAGAAGCCGA
GGATCAGAAAGAGGCTGCTGAAAAAGCAAAAGCAAAAGTAAACCAGGAAA
ATATGATTTTGAAGAATAGGATTACGCAAAAAGATATACATTTAGGTAGT
GAAAAGAAAAGGAACATTTTTTTGAAATCTTCATTATCTGAAGATAAAAA
AAGTTTCTCTCAAAAGGCGCATATGATACGAATTAATGTAAAAACAATTG
AAAATACTACTTCTTTTTTAGTTAATGAAATTACAAAAGAAAAACCTAAA
TTTAATATAATTAAAGAAAAATTGAAAATAATTTCTCATAATACAAACAG
AATTAAGAGGATTATTTCGTATGTGGATAGTGCGAAATTTAATATTGATA
ATGAAAAAACAGAAGGAGATTTAATAGGCTTTTTCGAGGAATATGTTGTA
AACATTGCCAATCAAGAATGGGAAAAACCGCAAGGGAAAGTTGTTAATCC
GGGGAAATGTTCCTTAATAAAGGTTTTCTCTCCACAAGATATAGGAGTTC
TTATTGATAACTTATTTAGTAACTCAAGTAAAAATAAAGCAAGAAATATA
ATTATTTCAATAAAAGAAGATAAAAAATATTGTTATTTGAATTTTAAAGA
TGATGGTAATGGATTACCTTCACATATAACGGATACAGCTGAGCTTTTTG
AATTTGGAAAAAGCTATACTATTCGAGGGACAGGGGTAGGTTTAGCACAT
ATTAAAGATATAGTGAATGATATGGATGGAAAAGTATATATTCCTAAGGA
TAATAAAGAAGGGTTTGAAGTAGAGATGAGATTAACAAAATGA
ORF2817 (SEQ ID NO:69)
ATGAATAAAGAAACCGAACGAAAGCTTGCTGCGTCACTCACTTCTCAATC
AATAGAAATATTACCGTTTATTCCGTATTTTTTACAAGATTTTTTTGAAT
TGGGTAGTAGTCCGAAAGATATTACTTATTTAATTAAGCAACATATGCCG
TTATCAGCTGAAAGTAATTTTTTGGATTTAGCTTGCGGAAAAGGCGCTGT
TTCCATTGGTATCGCTAAGGAACTAGGAAATCCTGTCAAAGGAATAGATT
TAATTCCGGCATTTATAGAAGAAGCAAAATGCAAAGCGAAAGAAGCGCAA
GTAGAGTCACTGTGCCAATTTGAAGTAGGAGATGTAAATAAATCGGTTCA
AAACGAAAAAAATTATGATGCCGTTATATTTGGTGCAGCAGCGGATATAC
TTGGAAATCCAGCAGAAACGTTGGAAAAATTGCAGGGAACTGTTAAAGAA
GGCGGTTATATTATTATAGATGAAGCATATGTGCCTGAATTAGCGCACAA
TAACCAGGTGAAGTATCAAAATTATGAATATCTCACTCGAAAGGAATGGC
TGAATTTATTTGAACAGAATCAATTACAATTGGTGGAAGAGCTAGAGGGA
ACAGCAGAAGTGGATTTTGAACTGGAAAAGCAACACTTATTAGCAAGAGC
CAATGAATTAATTCAGCAATATCCAGAGAAAAAAGCCCTTTTTGAAGGAT
ACCTTAAGAGCCAATGGAGCGAATACGATGACTTAGAGGAATATCTTATT
GCTGTTACCTGGATTTTACATAAAAAGTAA
ORF2819 (SEQ ID NO:70)
AAGCGATATGCAAGAAATTTTTCAAGGAGGAGCAAAATGCCAAAACTCGT
TACGGGATATGAGCGTCAACAAACTAAAAACCTAATTATCGAACATACAA
GCCATTTAATTTATATAAAAAAAGGAATTCAAGGTTTTACTGTGGAGGAT
ATTACTCGAGCAGCTAGGATTGGAAAGAGAAAATTCTATACGTGTTTTCC
CTCCAAAGAAGCATGCCTATTTGAGGTTGTGGAATATTCCTATCAAGCAC
AATTAGAGGCATTTAAAAAAATTATGGAGGAGAAAGGTTCGTTAAAAAGC
AAGATGACTCGTTTTTTGAAAGAAGTCTATCTTTCGGAAAAGAGTATAAA
CAACTACTTTTCACCGGAAGATTTCCACGCAATACTCCAAAAACTCCCTC
CAACATATACGGAAAGAGAAGAAAGAATGACAAGTGAAGTCCTGGAAACT
GCCATGACGTATATCGATCTTACGAGAGCTCAATGGGAGGCTTTAGTGAT
GTTACTAGATTGTTTAACTTACACTGCGACAAGGAGTTATGTAGAAACTG
CCAAAAAAGCGAAAGAAGAGACTTTGGATATATTAATACATTCTATAGCT
GATTATGTAGAAAAACAGACCCAGTGCTAA
ORF2836 (SEQ ID NO:71)
GAGGAAAACAGCTTAATGAAATATATGGTGAAATGGAGAGGTTTTTTTAT
CGTTGCAATTATTGGTTTACTCGTTTTTCAAAACGTGTCGCCTGTATTAG
CAACCATTGTTGATGAAAAAACAACAATGATAACGCTTAAAATAATCAAA
GAAGATAAAGATACAAAAGAAAAAATCAATGGTTCTTCTTTTGAAATTAA
AAACAAAAAAACTGGAGAAACAAAAGAAGTTTCTATAACTGAACACGGGA
CAATTATAGAAAATTCACTTTCAGAAGGAGAATATATTGTTAAGGAAAAG
AAGGCTGCTCCAGGATATACTTTAGACGAACAAACTTATAACGTCACTTT
AGCTGATAAAGAAGAGGCTATAACTTCTAGTTCAACAAAAAAAGAGGCAG
AAAAAACTCCATCTGTTACAGAACAACCCTCTAAAAAAGGGAATCTGAAA
GCAGTTATAACAGATAATATTTTTACTGCAGTAAAGGTGGAAAATGGAAC
TGGAAATGAACTTGGTGCGACTAACCGTATAAAAAATGGCGGAGCAGTAG
TTCTGAAAATGAATTTTACTTTCTCAGGGAAAAACTACAAAGCTGGAGAT
ACATTTAAAACGGTTTTACCAGATTCATTCAACTTTGGAACGACTAATTT
AACAGGAGATTTCTTACCTTCAACTGAAGCGAAATGGGATTTGAATGCAA
GCACACGTGAATTAACCATTACTTTTTTTAAAGATGGTGTGCAAGAAGGT
AATTATGATATTGAGCTTAGTACTGCTTTAAAAAGTTTCTCTGAGACAGA
AAAAACTAGTCAGGTAGCGGTGTTTAATACAGCGGGTGGTAATACAGTTT
ACCAGTTAGAGATTATTCCTGAAGTAGACAAAGCCACACAGGTAATGCTA
GAAGCGATGCCGAGTAAAGTCAATCCGGATAAAGCTACTGTGGATGCGAG
ATTTAATTTAACTAAAGAAACTAGTGAACTAGGCGAACTTAGACTATCGG
ATACTGCTTACGGGGGTTCGACTATTATTAATAGGAATAGTATTAAGGTC
TACTCAACAGATATTAGTGCTAAAGGAACATTCATTGGCTCAAAGCAATT
ACTTACTGAGAATACAGATTATGAATTGATTTATGCACCTTCAGGATTAA
CAATTAAATTAAAAGAAGGTCTAAAAGCAAAAGGGTATCAAGTTACCTAT
GAGCGTTCCATTGATAAGACAAATTCGTCTTTGAGTACTATCGGGACTTC
AGCAACAACAGTTGGAAGTTCTGGCATGTTATCAAATGGAAGTATGACCA
TTTCAGTAACAATAAAAGCATATGATCATTTAATTAAAAAAGCAGTTTAT
AACCCTGTAACTCAATGTATTGATTGGACAATTAATGTTAATTATGATTT
AGCAAACTTGACTCCTGGTACGGTTTTAACAGATGTATTAACAGACGATA
ATGTTAGTTATGTTGCAGATTCTTTGAAGATTAAGCGAGTCACTTTTAAT
GAAGAATCAGGAGAAGCAGTAATAGGCGATGATGCGTCTAATGATTGGAC
TGTTTCGACTATATCAGACAACGGTAGTTTTAATATGAACTACAAGAAAA
CTGATGAAAAAGCATATCAAGTAACGTATTCTACAAAACTAACCGATTTT
AGTCCACGAAAAATTAAAAATGAAGTAACGGATGAAAAAGGTGTTAAAGC
AACAGAAAACTTTGATTTCAAGCCAGACTTACTAAATAAAGAAGCTGGAG
AGATTGATTATTATAATAATACAATGGATTGGACTATTACAGTTAATTCG
GAAGGTATTAATATGCAAAACATTAATATCGTTGACGAGTTTTCTACAGG
TGTAAAAAGTTTAGTCAGCTACAATGTGTACGCTTATCCTTCTGATTCAG
GCTACAAATTATTAACAGAAGGTAGAGACTTTACCATCCAAAAAGACGTC
TCGCCAGCTGGGTTTAAAATTAAACTTATCGGTAACTATGCGACAACAGA
TAATAAAATTGTTGTGAAAATGAAAACAAAAATTGATTTAACTGATGGAG
CAAAAACGCTAGATAATAAAGCCTCGTTTTCATATTTTGACGGTAGTTTA
ACCCAGTATTCAGAAACAGTAAAGGCAGAAGCAACACCGGAAACTAGTAT
TTTAGCTAACGGTGGGAAGGTCGGTAAATGGAATCCGGCAACTGGTGAAA
TAAATTGGATTGTATCTGTCAATGCAATGGGGAAAAAATATGATAAATTG
GTTTTAGATGATGAATTTTTAGATGGTACAACCTTTGTTGAAGGATCTTT
ACAGTATCGTAATGTAGTTAATTCATCCGAGCTGACCGACTTGAGTATTC
CTCTAGAGATAAAAGGGACTTTAGCACAAGTTGGGGATGCTAATTATCCA
ACCAAAATAGACACATCAGCCAATAAAATACATTTGGAATTTGGTAATTT
AGATACTAATCGTGTATTTGTTAAATATAAAACAAAACCAAAAGATAATT
GGTTCTTCTCACAGTGGGTAAACAATAAAGCTATCGTCTCAGATAATGGA
GCAGATGAACAAATATACGAGACGAAAGAGTTTGCTTTTTTGCAAAATGA
AGTTATTAAGGTAGCTGGAAACATAGATAATGTCTATGGAAATAAAGTGA
ACTGGAATATGGAACTCTTGAATATTTCTCCAGAAAGAACACTGTCTAAT
CCGGTTATTACCAATCGATTGGAACAAGGAAATACGGGCGCTCAGTTTAT
TAAAAATAGTTTTCAGGTAATTAATACAAAAACGAACGAACCGATAAACG
AAGAAAATTATGATATTACTTTTGAAGGAAATACCTTTACCATTCAATTT
AAAAACTATACTGCAATGGCGCCAATAAAAGTAAGCTACAGCACAATAAG
TTTACTTTCAGGACCAATTTCTAACGAAACGACGGTGGAAGCAGAAGATT
TTAGTAATGTTCCAATGTTCTTTAAAAAAAGAAATGCAGCAGTATCACCA
GTCTTTACAGTGGGATCTGGATCAGGGATTGCAACGATTGGCACGATTAA
AATCACAAAAGTGGATGAAGACGATACTACGAAGAAATTAGAAGGCGCAA
AATTTCAGCTTTACACACTAGATGGTGAAAAATCTGGACAAGAAATAAAA
ACTAATTCAGAAGGTGAAATTCTACTAGATGGTATACAATCTGGGAAGTA
TAAATTAGTTGAAACAGAAGCTCCAGAAGGATACAACATTAGCGATGAAT
ACAAAGAAGGAAAAGAAATTACTGTTAATTCATCTGGTGAGGAACTTCTT
TTAACCATCAAAAATGCTATGAAAAAAGGCAAGGTTATTTTAACGAAAAA
GGACAGTGCATCAGATGAAGTATTAGCAGATGCCGAGTTTGAATTACAAA
ACGCCGCTGGGTCAAAACTAAAAGAAAAACTAACAACAGCTGCGAGCGGT
AATATAGAAATAACCGATTTAGCACCAGGCGACTATAAGTTAATTGAAAC
CAAAGCACCAGCTGGTTACCAATTAGACGCGACCCCGGTTCATTTCACAA
TTGATTTTAACCAGTCAGAAGCAGCGAAAGTAAGCAAAACCAATACAGCA
AAAACAGGCACGGTAGTGCTAACGAAAAAAGATAGCGCAACAAATACCGA
GCTAGCTGACGCCACATTTGAGTTGCGAAACGAGGACGGAGCATTAGTCC
GCGAGAATCTCGTAACAGATGATAATGGAGAAATTAGCGTAGCTGATTTG
GCACCAGGCGACTATAAATTAATTGAAACCAAAGCCCCAACTGGTTACCA
ATTAGACGCGGCACCAGTTCATTTCACGATTGATTTTAACCAAACAGAAG
CGGCTAATGTAACCAAAACCAACAAGAAAAAAATTGGTACAATTATAGTT
AAATTTATAGATGTAGAGGGCAATCAATTAAATGATGAGGAAATGCATAC
TGGAAATGTTGATGAAGAATACAATGTGAAAGCTAAAGAAATCGTTGGCT
ACACATTAGTTAAAGATTCCGCTAACAAAAAAGGTATGTATAAAGAAACT
TCACAAGAAATAACCTTTGTTTATGAGAAAAAGGCAATGCCGATTATTGT
GGAACCTACTGAACCATCAAAACCAACAGAACAGCTAACAGAATCAGCTA
CAGTAGCAGAGCCAAAACCTATAAAACAAAACTTTAAAACAACAAACAAA
TCAACAAATAATAAGAGAAAACTTCCTTCTACAGGAGATGAGTTCCCTTA
TACAATGCTATTCATTGGATTGTTTGTTAGTGTTGCTGGAGTATTCTTCT
TAAAAAAACCTAAACAAATAAAATAA
ORF2847 (SEQ ID NO:72)
GTGTCGGACTTGTTGTCCCTTTCCGCTCCAAAAAAAAGGCGTGAATTTAA
GATGCTAGATTCAACAAAACTAGATTCAACAAAATTAGATTCAACAAAAT
ATAAAACAAAAAATTATTTACACTTTGATTATAGAGTTAAAATTGAAAAT
GTAGAAAGCTATGTAACTGATCATTCGAAGATTGGAAATCATAGCTTTTT
ACCGTTAATACGTTATGTATCATCTTTTGAAAAAAGAATAGAAGAAAAAA
ATCCAGAGTTTGACAACCGGCCAATTAAGACTAAAGATAGAGTAATTATG
TATGCTGGACATATGGATAACTTTATTTATAAATATTATGCAGAAGTGTT
GAACAAGGATTTTTACAATAAATTTTGTATGGAAAAAGGTATAGATGATT
GTGTATCTGCATATCGAAACAATAAAGTGGGGAAATCTAATATTGATTTT
GCTGCTGAAATCATTAATCAAATGGTTAATTATAAGGAAGCTTATATATT
GGTGGGAGATTTTACTAATTACTTTGATAAAATAAATCACGAATTACTTA
AAAAGCATTTAGCAGAAGTGTTAAATCAGCCAAGGCTGTCAAAAGATTGG
TTTAATGTGTTTCGATCAATAACTAAATATGGATATTATGAAAAAAGTTT
TTTGAATGAGGAATATGGAAGTGATGAAAGTATCAAACGCTCTAATAAAA
AAAGTTATTTTGAAAATATTTCTAAATTTAGAGAGTTTCAAAAAAATAAC
AAAACTTTATGTAATAAAAATAAATTTGGTATACCTCAAGGAAGTGCTAT
CAGCGCAGTATTTGCAAATATATATGCATCGGAATTTGATTTGAAGTTAA
AAGAAATTGCCGACGAATTTTCTGGGATATATAGAAGATACTCAGATGAT
TTTATACTAGTTATCCCTAAAAGCGATATAGTAAATGAGCAAAAAATTAG
AAGGATTGAAACTGATACAAGAAGGGTAGCTTCGGAATATAAAATAGAGT
TGCATAAAGATAAAACAGGACTGTATTTATACGAGAATGATAAAATTTTT
GACATAATTAGCAATGAAGTAAGCCATTTAGATTATTTAGGTTTCGTTTT
TGATGGCACTACAGTTAAAATGAGAGGGAAGAGTCCGTATAAATTTTACC
GAAATGCAAAAAAACTTATAACGTTTGCTCAGAAAGTAAAAGTAAAAAAA
GAATTGACTGATTTACCTTATAAGAAAAAAATTTATGGATTGTGTACAGA
TTTAGGAAAGAATTATAATAATCACGGTAACTTTATTTCATATGCAAAAA
GAGCTCAAAAGAAATTTGATGAAATATCTCCAAATACAAATAATTTAATA
ATGAATCAACTAAAAAATCGAAAAAAGAAAATCGAAAAGATGCTAGGTTA
TAAAATACATACTAAGATTTAA
ORF2848 (SEQ ID NO:73)
CTATTATGTTATAATATAGACGATAACCCGAAAGGAACCACATGTATGAA
ACTTATAATTACCTTAATCATCATCTTATCAAACTGGTTAATTCAAGAAT
TGTTAATGATTCTTGAAGTTAACTTAAAGCAATTCTTAACCGACAATGCC
GATAAAGTATTACTTGACTGTTTGAAACATCATATTCTGTATTCTAAAAA
CACAGGCATGCTTATTCTATCACAAAATAAACATTTTTTAAAGAAAACAA
TGTATAACTTCAACTATTTATCCCCTACTTTAAATAAATATTACATTTCC
TATACAAAAAGAGCACTTTCTTAA
ORF2854 (SEQ ID NO:74)
AATATACAATATGATAATAAAAATTTTAAGGAGTTAAAGATGAAATTTTA
TATTAAGGAAATTCAGCTAACAGATTCTAACACATGGAAATTACAAGGCT
TCTCAGAAGGAAAAATCAATAGTATCCAAGCATACTACAACGAAATCCGA
GAATATAAGCACCCTGAACAAAAGTTGAATATTGCTTTTACTCAAGATAA
AAATTCATTCACAGCTACTATTTCTGTTGATGAACTTGCTAGTCTTTCTC
TACCAAATAACCAGACTGTGTGGAAATTCAAAGTAAATAATGATTATCCA
TATACACATTTAATCACAGATGGGCCAATTATAAATAAACCCTTCCAACC
AGAGAACTCGCTTTACAAATACCATTTTGATTTTCCTGAAGGTATTTTAA
CACTTGTCAGTAAACCTATAGAACTACTAGCTTCGATAGAGGAGTACAAA
TTAGATTCTGATGTGATGTCTGGTTCTATTAAAATCAAGTCGCCACTTCC
AAGTAACCAATTTAATGCAAAGCTAATTTTCAAACGTAGACCTACACCAA
GCTTTTATTTATTTCACGAGCAACAACAATCATTTGATTTAGGGCTTATT
ACTGAAAATATCGTTAATTTTTCTATTCCCACCAAAGATTTATCTACAGC
TTTCTTAGTAGACAATACAAATATACTAGATGCCATAATCGAAGTATCAT
CTAGTCATAACAAAACAGGCTTATCTGCATTCATATCCATTGATGCAGAT
ATGAAGCCAGCTATTCCTAGAGAAATTAAAATAGCAGCGCCACTTTTTGC
AACTCTGCGCTCCTATATTACAGGCTCTAACAGATTATCATTCTATTTCA
AAAAAAATATTCAGGGGTTAGTGAGTCTGAGTCAATTAAAAGAAACTAAA
AAAGATTTGACACTGCAATTCAAATTAGAAAACAGTATCTCTGAAGGTCA
AATAGTAGCTAAACGTGCAGATAAAAAAGCGAATACATTTGAATATAATG
TAGAACAAGTATGGCCTTTAAAGAAAGGGATTACTAAATATACTGCTCAA
ATTAATAAAAATGAATTCTTATCCGGACCAATCAATAGAGCTGATGCGAC
ATGGGATTTCTTCCTTCGTTTGGCAAACATGCCAGATTTACCTATTTTAG
CGCCAAATACTATTGACTTTAGTTCTTCGGGATTTTTCAATGTTGCTAAT
AACGAATTTATGGCGCAATTAACAAGAAACGACTCCAATAATTTAGCTTG
TCTTACAGCCGTTGCTCCTAAAATCAAACAAGATATAACAAAAATAGCTG
TAATGGGAACTTGTTTTAGCCGAAATGCTTTTAACTCATCTCCATTCTTT
AATCCAGATTATAAAGCTTTTTTTGAATGCAGTTTTACGCAGTTTCATTC
TTCTATAATTAGTATAATGACCGAACCCGCTAACTTAATTAATCTAGATA
AGTATACTGATATTAAAAAAAGCGAGAAACCTTTTATAGAAGATGATTGG
AAAAAAGATTTCTTTACTAACCTGAAAAATTCTGACGCTGATTATTTCTT
AATTGACCTATATCCTGATGTTATTCGACCAGTTATTTGGTTAAATAATA
ATTCAGCTATTACATTATCTTATGTTATAGAACAAAGCCAATTACTTAAT
GACATTTCTTACGAGAGAATTTTAGATCATATTGATAATGAAACCTACTT
CAATGAGTGGAAAGGCTATGCTGATCAATTTATCGAAAAACTTACAGAAA
TAATTCCTACTGATCGGGTTATTTTAAACCTAGGTGGATTTACAACGTCT
TATTATGATGAAGATGGAGAAGTCGCAACTTACAAAAACAAAATGGCCAT
CGAAAAGAATAATTACTTCTGGGAACGTTTAAATAATTATTTCTTATCTA
AATTGCCTGAAGCAAAAGTAATCGACTTTTCGAAAAAAGGCTATATAGGT
GATTTCAACTATCCATTTGGGCATTCCTTTTCTCACTTCGAATCCCCTTA
TTATAAAGACTTCTTGAAGGAACTTATTTACATCACTAAATCTTAA
ORF2884 (SEQ ID NO:75)
AGTAAATTGAAAATGGGAGTTGAATACTTATTGGCTTTTATCAGAAAAAA
TAGCTTTCTACTGTTTTTAATATTATTTCTCATGAGTTCTATTTTAATTC
ATATGTTTTTTTATAATTTTCTCACAGATATTATTGTAACTGTGTTTTTG
GGATTAGAAACCATTTTTATTGTCATTATCTCACTAGAAATTATGGATGT
TAAAAAGATAAGAAAGCGGACAAAAAGAATTATAGAAATATATTTTTTAA
CCTTACTTGGTATAACAGTAATCACGTCATATTTATTTTATTAA
ORF2885 (SEQ ID NO:76)
ACATTACAGGAGACAATCATGATTGAATTTATTATCGATGTATCAATAAA
TTTTATTACATTTGCAATTTGTTTCATTCCGCTGCTTCTCTCAGAAAAAA
CTAAAGGTATCTTGGAAATCGTAGGTGCAAGCATACTTTTTGCGGGGATT
ATGATAGTTGGAACAGGTATTTTCATAAGCAGTAGCGAAACCCTGAAATC
ATATATTTATGTGATTTTGGTAGTGCAAATAATTATTTTGTGCAGCGAAC
TCATATTAGTTTTATGGAGTAAAAGTAAAGGAAAGTCTACAATTTTATCT
ATCCTATCAGCAATATTAGGTATAGTCGCTTTAGGAATATATATTTATTA
TGTTATAGCAAGTTTTATTTATTGA
ORF2886 (SEQ ID NO:77)
AAACAATTTGAAATTGCTGCTGATTATGTATACCAAAGTAAAGAGATAAT
CTTTATTGACATCATAGGCGACGAGACGCCAGCAATTGGAATAATCGCTG
AAAATGACTATGAAGGATTAGAAATTACTTTAGTGGATGACGAAGGTCGA
TTAGATGGAACTTGTTGGATAAAAAAAGAAGATATTCTAGCCCTGCGTTT
CGGCGGCAGTGTAGAACAAGATTTGATGAACAAACTAAAAAAATAA
ORF2887 (SEQ ID NO:78)
AAAAGGGAGGCTATTCGCTTTAGATTACGGAAAAGTTGGTGGAAATCAAA
AAAAATATTTACATGTCCACTGGAACTACAAACCAAAGCACTATATAATC
TATCCAAGAAAAGGAAGGGGAAAATAATGCTTGACAAACAACAACTATAT
ATTGTAGAAAACAAAGACATACCTAATTATGAAGTTGACTCTGGAATTAC
TGGAGCAGAACAAAATTATATTTATAATTTTAAAAATCCAATCCCTATTT
CTGAACAAGAATTACAAGACATTTGTGATACTTTTGGTGAGTTTATGGTT
TCTGCTTATTTTTTAGTGGATATATTTGGGCCGTATGAGGAATTACCTGG
AGACAAAAAGAATGGCTATAAAAAAGTTGTTCTTGCAGATGAATCTATGG
AGATAAAGAAAAAGATGTTTCATTTGTTTTTAGAAGGTGATGCTTATCCA
GACGCAAACAAAATATTTTTGTTCAAGGAGAAAAAAGAATTAAATGCTAG
AAAAAACATGGAAATAGAAGATGGAATGATAGTGATTTCTCCTGTTTTAG
ATGTTACTGCTATAGGCATATTTTCAAAAGGAATAGATTTGGCGCAGTTT
TTCAAGTAA
ORF29 (SEQ ID NO:79)
GGGGGCTTTTCTGTGTTACAAAGAAGATTTTGGGGTATTTTTTGTTTTGC
CATATTCTTGTTTTTATTTCCAACTATTGGTTCTGCCGAAACATCTGGTG
ACTATGAATATACAATAAATGGAAATGAAGCTACCATTACTGATTATACA
GGACAGTCTACTGATATTACCATTCCTACAACACTTGGTACTAATAATGA
ATATACCGTTACTGCTATCGGGAATGGCGCATTCAAATCCAAGAGATTAA
CTAATGTAACGATTCCTAATACAGTTATAACTATTGGCGATGGTGCTTTT
ACCATCAATTCACTTGAACAACTAGTCTTACCCAACTCAGTGCAGACTAT
AGGGAGGAACTCTTTTAGTGTCAATAAATTAGAAAAAATAACTTATTCTA
CTGCTTTAAAAAATATTCCTTCCCAAGCATTTTTAGCTAATAATTTAAAA
ACAGTGACTACACCAGCCACTGTTGAAAGTATTGATGCTTCTGCCTTTGA
AAATAATTTTATTACTAATATTACAATTCAAAACCCAAATCTTCAAATGG
CTTATCAAGCTTTTGCCGCTCAAACAGTTTTAAGTACACTCATTGTACCA
AGCAACCACATCCTACCTATAGAAAATTACATTCAGTTTCAAGATGCTTC
GGCTCATTTGACTACAGATAATTTATTCATAACTGATTTAGCGAATGGTA
TTACGTATAATCAAGCCGAAAAAGCATTAAACTTTTCAGCAGAGCCACTC
GAATCTACTTTTTCACTTTTCACTGGGACAAATCGATTTGATTCTTACTA
TGATATTTCTGAATATGGTCCTTCTGGAAAGCCATTTATCTACTTCAAGT
ATACTAAACCAGTCCTAGTTTCCTATAAAGATGCGTCTGGGAATGAATTA
GCTACTTCTACTAGATTAGATGGTAGTATTGGTGAAAATTACGTCACTAC
TCCTAAAATAATAGATGGCTATACTTTAAAAGAAACACCCGGAAATGCGA
CAGGACAATTTTCTGAAACACTGCAAAATGTAACGTATATTTATGAAAAA
ACTGCTGTCCAAAATGGGACAGTCACTGTAAAATATCAAGATGAATCGGG
TAAAACACTAGCAAAAGATACTGTTTTAACCGGTGAAGTAAATAATACCT
ACCAAACTAAAAGCAAAGACATAGCAGGCTATAAACTTCAAAAAGTGGAA
GGAAATGAATCGGGTACTTTTAGCACAACCCCTGCAACTGTTACCTATAT
TTATGAAAAAATAGCAAACAGTGATAATACAAATACCAATGGAGAAATGA
CCGACAATACTACACTGAGTACTAATGATACTGTGATTTCATCCGAAGCA
ACAAAAAAAGTTGATAAAAACACCTCTAATATCCTCCCAACTACTGGTGA
TTCAAAAGATGCTCTCTTTTTTGCACTAGGAAGTTTATTGACGTTGTTAT
CTACTAGCTTCTTTTTCTTTAAAAGAAGTTAA
ORF2925 (SEQ ID NO:80)
ACAACAAGATATACAAAAGGAGTTGTTCCAGTGAGCCGAATCGACATCGG
AGAAATACAAGATTTTGCATTCCAACTACGCGCAGCCAACCAAACAGGAA
GAAAAATCATCCAAGGCGTCAAAACCACCGTGACAAACTACGTAGAAGAT
GGTAGTTTAAAAGGGAAGGCCGTGGAAGCGTCCAAAAATTACTTTCAAAT
GACATACATTCCACTCTGCGACACGATAATCGAGGCAATGAATGAGAGTG
AGGAAAGGTTGAAGCGGTATATCCAAGACTTTCACGACCAAGTAGACCTT
TCTCCTAATGCTAAAATTGATGCAGATGGTTTATATGAACTCGGCCAAAT
GATTGACCGCATTGAAAGCAAAAAAGAAGCACTGTACCAACGAATGAACA
GCAGTACAGAAGGCCAAATGCAAACCTATCGTTCCCAGTTAGCAACCGCA
TATAAGCAAGAAAATATTTTAGAGAAATATCTGGCTTTTGAACAAAGTCA
TGGGGCTTTTTTCGACCATTTGACGGATTTAGTGCAAGGTATCCAGCAAA
CTGTTCGAGAGTTACAATCAAATATCCAGTTTGATAGTCAAACGGGCAGC
TATGATTTAAGTAAATTAAATTTTGCCACTGTGAACCGAATGCGAAAAAC
GCTAGGAAAAGCGAGTGCCACTGATACGACAGTCTATAATTTTGCGAGTT
ATAGCAAAGTAAAACAAGGCGTTATGTGGATTCTTTCAAAAGATGGGAAA
GTGGATATTAAAGCAACGGAGGCTTATAATACAGCCAGTTTTAACGGTGA
GCTACCAAAAGAAAGTAACCAAGCCACGGAAGAAGGCGAGTTGTTAAAAG
CCACACTAGAATCACTGAAGCAAAATAAAGATCCGATAACTGGTCAAGAA
ATAATCAAAGCTCAAAGTTTTGGGATTTTAACCTCACTTGTTTTTGGCTA
CACGACGAAAGGGTATCAAGGGAAGAAACTGACAATATCGAAAAATACAT
TGATAAAATTGAGACAAGCTGATAAAACGGTTATAGAAAGGATTAAGAAA
ACTAGCAGAATTGACTATGAAGAAATTGTTAAAAAAGGTAGTAAAATGCC
TAAACACATCATCGTGGAAAACAAACAAAGTCTTCCGGGAAAAGCTATGC
CCAGTTCCTCGGCTGATTTGTTAAATCCTGATGGAAGTGTAAAACAAAGA
AGATATTATGATGAAAAGGGCAGAGCTAAAGAAGATATAGATTTTAACCA
TTCAGACGACGGAACTCACGAATTTCCACATAGACATGAGTGGGATTGGG
ATAGAAAACCGCCAAGAAAGCCATCTAAATAA
ORF2927 (SEQ ID NO:81)
GTGGATTCAAACAACGAGAAATTGAAACAACAACTACAAACTCTCCAAAA
ACAACAAAAAGATGCGGAATTATCCTTATATATGCTCAAACATGAACAAA
ATGAGCGAATTTGGCTAGAAGAAGATTTTGAGCGGATTTGTTACGAAGAA
CGCGAGTCTTTGGAGTTGATGAGAGAAGTCTGGCAAGGCGATCAAGCTCG
CAATTTCGGCTACTATTTAGAAGACCTCCAAGCAGACGAGAAAAATAAAT
GGTGCCAAACCTTTCAAGCTGAGGAAGAAAAACGCCAAGAAAAAATAAAT
ACATACCAAAAAAACATCTACCAATTGGAAAGTAAACAACAAGATATACA
AAAGGAGTTGTTCCAGTGA
ORF2969 (SEQ ID NO:82)
TATATAAATCCAATTCGAAAGGGACTCAATAAAATGAATTCAAATGAAAT
TTCAGAGATTATAATTCAGCAGAATAAAGGGAAAACAATTATTAAATTGC
TTTATACCTTGCCTATATTATTATTTGGAATTTTTATTTTATTTAAAGTA
AAAGAATCGTTATTACTAGGTGCTTGTGGAATAATCCTTTTAGTTATCGT
GGCACCAACTTATTTTACCGTAGCTAAGCATTTTTTTTCGGGTAAACCAC
TGCTTAAAGTGGATCAAGATGGTATTGAGGGATGGAGTAAACGCTTTAAG
TGGAATGAAATTGAAAAAGTAGCCTTGCGTAGAGACTGGGGCGCCGTATA
TCTGACTGTATATGTACGGAAGAATGGCGGAATTCACAAATATAATATAA
ATACAAAAGAAATAGAACGTTCTGCCACAGAATTAATCAAACAAATAGGT
TATTTAAAAAACAAATATGAATGA
ORF2970 (SEQ ID NO:83)
AAATTGGTTAAAAGGATTAGAATAACAATGATTGCCATCACTTTATTTTT
ATCGGGATGTGAAAATGGGGATACCGAAAAAACAGATACAGAGGAACAGA
CTAAAAACGTTGAAGATGAAGGGGAAAAAGTTGAAATTGAGAGTAATGAA
GGGAAGCCACAGCATGAACAGCTTATTAAAATAGAATTGCCTCCTAAAGC
AGATTATTTAAATGATGAAACGTTAGAAGTATACAATCAAGCTAAAAAGA
AATATGACCAGACAAATCAACTAATTACTAATGATTCAATCACAGTACTG
GTAGGAGATTATGGTTACTATGATTCGGTGTGGGGATCATTAGATTGTTC
TGCAGTTATTAAAAATGGAACAAACTCAAGCATTAAAGACCTTAGCTTTG
AAGTTAGTGTAGAGGATAATGCAATATCTGGAAAGACATTTCTAAATAGT
GAAGCACTACCACTTACCAAAACTCAGATAGGAGATTTTGAACCCAATAC
GGGAGTGCCAATAGTTATAGCGTTTCCAGAAAAGAACGCGACAGGTGAGG
GTGAAGATAAGAAAATTGACACTAAAAATGTAAAGATTCATATTAGTAAT
ATTCAATATAAAGTAGAAAAGTAA
ORF3102 (SEQ ID NO:84)
ATGAGCGGAAAAATAAAATTCAATATCGCCGAAGCTCAAAATATTAGTCT
TGAGCTGAAAATTGCAGCAGGTAGGTATACGCAAGAAACAGAAGAGCTAT
TAAAAGTGCTGAAAAATAATAGTTTATGTGATAAAGACCAAGATGTCGTT
GAACAGAGGGGGCGAATAGAGAAGAATAACCAAAGACTTATAGAATATGA
GAAATTTGTAAACACAAATCTCGCTAAATCTAATTCAGTTATAGAAGAAT
TGTTTATGAGTGTAGAAGTATTGTATGCACAGCAAGTTTCTGAGTTTAGA
AACCCTAATTCTGCGGGTTATAAAGAATTAATGGGAAATGTGAAAGCGAT
ATCATATCAAAAAATTTCACAGATTTCAGGATTGGGAGAAATGCTTACAT
CGAAAGGTTCGGTGGGAGCAATTAAAGATATCAGAGACACACTACTTGGA
ACATTGGTAGATGATACTTACTTGAAATTAATTGATGATACAGTTGAATA
TCAAGGAGAAAATAAAAATATAACTGATTTATATGGGGATAAAATATCTA
AAGTGGGGTCAAGGAGAAAAAAATTGGACACTCCAACCAAATACCTTTCA
ACAGCCTACCTCATCTCAGACACGCTAAAAAGTTTTAACAGTTATGGAAA
TAGCAAAGATTCCTCTCGACTTGCAGGAGATCTAACCGGTATAGCAATAA
CAAAAGGTGCGGATTTTGCTGTTGGGAAATTAGCTACTACGGCATTATCT
GGTTTTGGTGTAAGTGGTGTTAAAGGCGCTATCGCAGGCGCCATTATTTC
CGTTGCTGCAGATAAAATTATTGATCCGACGGTTGAATATGTTAAAGAGA
GCAAGGTAGAAGCAAAGAGGGATGACTGGGAAACAAAAGGTATATATAAA
GGCTGGAAAAAAATTCGAGATTTAAAACTAGAATATACAGTGGGTAACTA
TCAAGCAAGTTAA
ORF3103 (SEQ ID NO:85)
ATAGGAGGGAGAAAGATGGCAAAAGAAATTAAGGTAAATCCAGATTTTTT
GAAGAAGGTTGAAAGCAATGTAACAAATTACATTGATGCCCAGAAAGAGG
TTTCTGTTGAATTACTAGCAGTTAGGACCAACTTAGCAAGTAATTTTTCA
GGTATTGCTTGCGACGAAATAAAAAATTATATAACAGAATTAATGAATGA
TCTAGAAAAAGAATTTGGTGTATTTATTACAAAGAATCATGAAAAAGTTA
AGGCGTTAGGGGAAAGTTATAAAGAACTTGATGGTCAGTTGGGACAAACC
TTTAACTATGGAATGGAGAGGACTAAATGA
ORF3132 (SEQ ID NO:86)
GCGGTTTTTGTGCAATTTCAAACAGCAAGAACTGTCTTTTTATCTAACAA
TTCAATAAAAACCAACCTTCACCCAAAAGGAGCTAACATGAAAAAACTAC
AATGGCTAACTAACCGATTATTTGCAACCTCTATCCTCCTAATCACCACG
TTATTTATCATACCCCCAACATTTGCGATAGCTGATGGAAGTAAAGTGAG
TTTTTATGAATACATATATGGAGCACCTTTTAGATGGCTAACTGTAATTA
GTACGACAGATAAGAAAGGTGCATTTACGGAGATGTTTTTCTCGGGAAAC
GAGGGTATAACTATCCAATGGCCCAATCTAATGATAAACTTTCTCTTAAT
TTTCCTTGCAATAACTATTATCTTTTCCCTAGCAAAAAAGCTTTACGATA
AAAAGAACGTCAAAAAAGACAACCCATAA
ORF3135 (SEQ ID NO:87)
CAAAAAAGGAGACTTATAGTGAAAATAGACTTAGAACAAATATATACTTT
ACAATTCACAATGGGATTAAGATACAGACCAATGCAAATCAGGCATAATA
TAAAATTATTACTCGTGGCTCTTTTGTTTTTTTCCTGGATTCAAGGCGAC
TTTATGAGTGGACTATCAGGCGATTTGAGTATGTTTAGAGTAGACATGTC
ATTAATTGTAATTTTAGGAATTTGTTTTATTTTATTAAATCCAATAAAAC
TAGCAATAAGGTTTCAGAATTCCAATATTTGTTGTATATTTTTTGTTTTC
AATGGTTAA
ORF3136 (SEQ ID NO:88)
AAGGAGGAAAAAATGTATATTAATTTAAAATCCAGTGATGGAAATGTGAT
TACAACAAAAGTTGGTTCTTGCTGGACCTGTGTATTTCTTTCGTTCTTTG
GTCCTTTACTCCGAGGAGATATTAAATTCTTCGTTCTTTATGTAATATTA
GATGGAGCGGGGCTTTTCATCACTTTGAATTATGATAGAGATATCGGAAT
AGCTATCATGGCTATGGTAACTTTACTCTTTGAATCCAATTATAATACAT
GGTTTATCCGCGGGAAGATGAATAACGGGTGGGAACCAGAAACAGAGAAA
GATCGAGAAATATTGTTAGAAAAAGGCGTCATTAAAACAGAGGTATAG
ORF3137 (SEQ ID NO:89)
ACTATGAAAAGTAGAAATTGGTTAAAAGGATTGGGAGTAACAATGATTGT
CGTTACTTTAATTTTAACGGGATGCGGAAATGGAGACGCTGAAAAGACAG
ATACAAAGGAACAGACTAAAAGTGTTGAAGAAGAAGGAAAAGAAGTAAAG
ATTGAGAGTAATGAAGGGAAGCCACAGCATGAACAACTTATAACAGTGAA
GTTGCCGCCAGAAGCAGAGTATTTAAATGATGAAACGTTAGAAGTGTATG
AGCAAGATAAAAAGAAGTATGACCAGACAGAGCAACTAATTACTAATAAC
TCAGTTACAATTCTATTAGGTGATTTTTGCTATTATGAGCCGGCATGGAG
CAGTCTTACATGTTCTGCAATTCTTGTGAATGGAACTGACACAAATATTG
AAGACCTTAGCTTCAAAGCTGAAATAATGAATAAGCCAATGTCAGGAAAA
ATTTTTTCAGATGACAAAGTTCCAGAACTTACAAAAGCAAAAACAGGAAA
ATTCCAGCCAAACGAGGGTATACCAATTATTTTAGTTTTCTCAGAAGAGA
ACCCGAAAAATGAGGAAAATGCGGAGCCTCAAAAAATTAACATTAAGATA
TAAAGGTTAAGATAAAGGATATTCAATATAAAGCAGTTAAATAG
ORF3138 (SEQ ID NO:90)
AATATGGTAAGTCCAATGCCAGATAAAACAGTAAAAATGGATAAAGGAAG
TATTAATCCTAAATTAGATAAACTAATTTCAAGTCTAGACAATTTTGATA
ATATCAATAAGAAAATGGGCACTATAGTAGGTAGCCAGGAGATTAGTGGG
GAAGCAGCAGATGCTTTATCTGAGAGCCTATTAGTGCTAGTAAGTTATGG
GGAACAATATAGTTTAGTAATTAGAAACCTAAAAGAAGTGATAGTCGACT
ATTGTAAAAGTGTGGAAGAAATTGATAAAACAGCAGCAAACGCAGTAAGT
AAAGGCGTTTAA
ORF3140 (SEQ ID NO:91)
GAGATGATTATCATAAAAAATTTCGCATCAAAAAACATTAGACTAATTAT
GGCACTAGTTATTGGAGTATTGGTTTTTGCACCTGTTTTTCATTCAAAAG
CCGCTGATAATAACATTGGTTTCGATTTTAAACTCAAACCAAATTGCGCT
AACTCAGGGTCAAGCTCAAGGTATAGAGAGACTTCTTCTGTAAATAACCC
TTGGAAAGTTCGCCTCGATAACTCAACAGAAGGAAAGGGTACAATTGCTT
CTTTTTGGCTAGGTACATATAATAAAAACAAGAATGCTGTACAAGGCTCA
ACAATAATGAATGTTAAACAAGGTGCAAAAACACGTTATTGCGGCGCTTA
TAAAGTAGCTAATAAAAATACAACCTATTTAGCTGCTGAAAATAATAATT
ATAATTCCAAAACATACTATGTCGATGGTATTTGGGATGAGGAAACTTGG
TAA
ORF3141 (SEQ ID NO:92)
TTTGCAAAAAAAATCAAGAGGACGGAAGTGCTTACTAATATTAATTATTC
TTTTGAGCCTGGGAAAATCTACGGAGTTTTCGGAAAAAATGGATCTGGAA
AAACAATGTTACTTAGATCGCTTGCTGGCTTAATCATACCAACAAGTGGC
AAGATATTCATTGATAATAAAGAACTTCATCATGATATTTCTTTTCCGCC
AAGTATGGGCATTATTATCGAAAATATGGAATTACTTCCCCAATTTGATG
CGCGGACAAATTTAAAAATATTATCTAAAATTAAAAATATTGCAAGCGAT
GAAGATATTGATAATGCCATTGATAGAGTAGGATTATCTCCACATATTGA
TAAAAAAGTTAAAAAATATTCGCTTGGAATGAAGCAAAGACTTAATATTG
CACAGGCCGTTTTTGAAAAACCCGCTGTCATCTTGCTTGATGAACCCACA
AATGCGATAGATGATCAAGGTATTGAACTAATTAACCAATTACTTTTAGA
AGAAAAGAAACGCGGAGCCACTATTATTATTGCAAGTCATCATAAAGAAG
ACATAGAACCACTTTGTGATATGAGTATCCGAATGGACCAAGGGAAAATT
ATCCATGACTAA
ORF3142 (SEQ ID NO:93)
GTATCCGAATGGACCAAGGGAAAATTATCCATGACTAAGACCAAATTCGT
TATTTTCATAGCACTGACAGTCATTACACTGCTTCTATTCCTCGTTCCTA
AAGGTATTCAATATCTCAAAAGCCAAAATCCTGAGCTATTGAATACAGCT
GAAAGCATTAAACTTCAAGCTGGTGAATACACTGTTGGAAAAGATATAAA
AGTTGGAATATATGATATGCAAGTAACAAAGGGTTCGCTATCTTACTATA
GTACAAGGCTTTCTAAAGGCGATGAAATAATTGGTATTAATTTACTAGAT
GCTAATAAACTGTATTTTGAAGGAAGCGGTGAAGTCGAGCTTACACCGGC
AGAATTTAATCCCATTAAACCTTCTGCCAATATTTTTACAATCCAACACT
CAGGGTCTTACGAAGTTGGTAAACAAATTCCAGCTGGGAAGTACAGCCTG
ACTTATACCATTGACAAAAGTAGTAAGAAAAAACCTTTTATTCAAATATT
ACCATCTTATACTGATGACGCTCGTATTGAAATACAATTTGAAACCAAAC
CGGCATATAATATTAATTTAAAAACAGGAGAAATCTTGACTGTTAGTAAA
ACAATATCAGAAGAACTTGATACTATGACCGTCTTACTTAAAAAGAACTA
A
ORF3144 (SEQ ID NO:94)
AAAAACCCTATAAATAATTCTCAAAATTATTCTCAACAATTTGTTAATAT
TGATGAAAGTAATTCTGATGATAATTTGGGTAACTACGTTAAAATAAGCC
AAGATAACAAACCTATTGGTTGGATAGACTCTGAAAATGTAATTGATGAA
AAAACTATGGATTCTATAGAAAATGATGAAGAATCAATTATTCCGGAAGA
ATTAATTGATAAAGTAAATGATTATGTAACAATAGAAAACAATGAATTTG
TATTGTCTAACAAAGCGGAAGATGTTCTTACTTCGGAAGAGTTTGTTGAA
GTTGAAGGGCAGATAGATCGAACTAATGCTGAAATCGAAGCTGATGAAAC
ACTATCAGAAACACATATTGAGGGAAACATAATAGTTCAAGAAATCTATG
AAGATGAACCAAATCAACTTCTTAAGGCATCTAAAAAAGCTTATATAAAA
GCTAAATATACATGGTGGGGTATGCAAATCCAATTTTCTCATAAAGCTGT
TGTTGATTTTAACGATTTTTATTGGGGGGCAGGTACGATAGGTGGATTAG
GTGCAAATAAACGAATGGGGAAATTTTTAGCAAAAAAAGGAATAAAAATC
GCATCACGTTATGCTGTTTGTTTGAGTTTATTTGGTGGTGGTTTAGCATG
GGGAATGGGTAAAAAAGATAAAGGAAAAGGTGTAAATTTAAATTGTGTAT
TATATGTCCCTGCGACAATAACTACAGCTAAATAA
ORF3172 (SEQ ID NO:95)
ATGAGTAAAGAAAGATGTGCATGTTGTAACTGCTTAACAATTGATGTTAG
AGGCGAGTTTGAGATTTGTCCGATATGTTTCTGGGAAGATGATGCTTATT
TTGTCTTTGATAAGGAAGAGATTTATTCTCATTATCAAGGCAGTTCCTCG
ATAGAAGATTTGTTAAATATTCGTTCTAGCGCTAACAATAGTTTAACGTT
ATTAGAAGCACGGCAAAATTTTAACCAGTTTGGCGCTTGTGAGCTGGCAA
TGAAAAAATATGTAAGAGAGCCAACTGCAGAGGAATTATAA
ORF3173 (SEQ ID NO:96)
AATATAAGTTTATCAAGGAGGAACTCCATGGAAATCACATTACAACAACC
TACATCAGCTGACTTTCCCTTCATTGAATGGTTGTGGGGAGATTTGGCAA
CGACAGAAATACTCGGTGGACCATTTTCTTTTCCGGAAGAAACGCGAATG
GACTGGCTAAAGTCAAAATCACAAGCGAGCAATGCTTATTTTATTATAAA
AAAAGGTACAGAATCCGTTGGTGAAGTCAGCTTTCGCGATTTTGAAAAAG
GGACAGCTCATTTAAATATTAAAGTGGCTGCATGTTATAGGGGCCAGCGA
ATCGCTCAAAAAGCTTTGCAATTATTCTTGGATTTTTTTCAAACTGATTG
CGGTGGGCTCGTTTTGTTGGATGAAGTTAGACGGAAAAATGAAGCAGGCA
TTAAGTTTCTCGTGAAAGCTGGTTTCGAAGTTATAGAAGAAAAAGAATGG
ACGATGGTGCTCAAATGGAGTGCTCAAGCAGAAGGAGGTTTTGAATGA
ORF3174 (SEQ ID NO:97)
AAAATGAATACTTTGTATTATAGGGTGTCTAAAAATACAAATTTTGAAAC
AGCAGCAAGAGAGATTTTTGATTTATTAATAGAAACTCAACAACAATTTG
AGAATCAACCGAGATTTTTAAAGGTAGAAATTGATGGACATTTAAATGAG
TTAAATGAGTTTGATAATGATATGCTTAAACTTCAACAAGAATTTGGAGA
AATTTTTTTACTCCAATTCTTTACCACGATTAGTTTCCCATTGCTTACAA
AGAAAAATCCTAGAAAGCAAATAAACGATATACCGAATGACCTAACAATT
TATGATTTGAAGTATGGAAATCCAGCATATAAACTTCAAATTGAAAACTA
TTATAATACTGAATTTGTTTTAGAAAAAGATGTGTATGTATTCTTGGAAA
AAGTTTCTAACCTCCTAAAGAAATATGGAAAAACAGATAACTATAAAATC
AATATTGAGAATGAGGAGTATGATAAACTTGGCTTATTGATTCACTGGCA
ATCTTATATGAAAGAATTGATAGTAGAACTATTTAATTCTTTTGTAAACG
GTAATTTAATTTCGAATGCTGCAATGACTAGGTCACTTATTGAGGGTTAT
GTGTATTTAAGTATTATAAAAAAAGAAAGAAATCCATTGTTAGTTCAAGA
TTGGTTTCTATGTAACTTAATTATTGGCAGCAAAAGATATGATGATGGCA
CTAGAGAAATTTTGAATAATACTTTAAAAGAGCTATTTGACAGTAATGAA
GATATACAGTCTAGATATAAAAAAGGGAACACTAATAATTGGCTATCAAC
AGTAATTGCTAAGAAAAACATTACATTTAGGGATGCTTGTGAGTATTTGG
AAGAAGATTATTTGTACAAAGATTTCCAAGAAGCAAGTTCTTTTGTCCAT
GCACAGGATATACAAACCAAATTAAGCCCTTTTTTTTCATACTCCTCGAT
ATACGGCAAACTATATATAATGATAATCTATATGTTTAAGACATTATTAC
TATTTGAATCATCTTTAGTATTAAAAGAGGAAATTGCTGATTTAGAGCTT
GAACTAATAATACTAGGTGAAAATTATTTATAA
ORF3175 (SEQ ID NO:98)
CTTTATTCTAAAACTATACTCATCTTGCTCATTTTTAAACACAATGTTAT
CATTAAATTCAACATAAATTCAACCAAGCAGGTGTGCATCATGATATATT
ACAAAGACAAACTTAAAACAATTGGAACAGCTTTAACTTCTTTATCTTTT
ATTTTCATGTTTGGCTTATTAACTTTATTGTCAATAGAAAAACCAGAATT
ATTTTACTATGGTCTTATTACTATATTTTTTATAGGATATTTAATTGTTA
AACTTATACTCCCGCCAAAAGAATGGATCCAGGTCACAAAGACTGCTTTT
ATTCTTCATAAACGCAACGAAACAAAAACGATTCATCTTGCAAATGTGAA
AAGAATAAGTTATAACTTCCAACCTTCTCGTCCGAGCTTTGGACATCCTA
TTCCAGAAACGATGGAACTATTCTTTCATACAGAAAATAAATTAGAAAAA
ATTGAATGTGATTTTATCGGCCGTACCAATTTTGTTGCGTTAATTGACCA
ATTTGATGCCAAATTAGCCTCATTAAGTGAGGATATAAAAAACCATGACT
ATAGCTATCCACGCGATCTTACATTCGGCATTTTTATTTTTAGTATTGTC
ATGCTCTTCTTGATTTTCATTCTTTTATTTGGTCGAGAATTTCTATTTGA
GCAAATTGGGAAAGTTTTTGTATAG
ORF3183 (SEQ ID NO:99)
GTTTTCGGCTTTTTTATTATTGGTGCTTTGAAAATATTAAAGCAGATTAG
GAGGAATAATATGAAGTCGCAGCAGGCCTTTATGGTAGAGGTTACCTTTT
ATTCTAATGCAAGAAAAACGGTTCCGGAAGCGGGGTATCGGCCGCATTTT
GTGATGGAGTTGGATGAGAATCGGGAATATTTAGGAGTTGAAATTTATGA
TATAGAAGTGGATACGCTAGATTCGGTGGGGTATGCAATGTGTTCTTTTT
TATATGAACAAGAGGGCGTTGGTTATAGTAAGATTCAGCCAAATAAGTCT
TTTATGGTTATGGAGGGCGCGAGTGTTGTTGGGAGAGGAAAGATAGTGTC
GTTTCATTTGTAA
ORF3184 (SEQ ID NO:100)
GTAGTGATTAGTATACAAAATAATTTGGAATATAACAAGTTACGTTGGGA
AACTTTGAGTGGAAATGTAACAGGAATATGGGAGAATAATAAGTTCTTTC
TAGGCTCTAGTTCTTACCCTATTATGAAGTATCATTACATTACAGCTAAT
TTTGTTAATTTTGAAAAACATATTTCTGAAAATATGCCTAAAATAAGTTA
CCATTTAAGTGGATATGGTGTTAATTTTAATGAAGCGCTTGTTAGTTTTA
TTGGTGAAAGCGCAGAGCGATATACATATTCCTTACTACCTACTATTATT
AAGGACAGGATTATTTTTAGATCATATGAAGAAATGACAAAAGAATATAA
AACTGACTTAATATGTGAACTTAAATACATAAATTCGTATTACTCTTCTG
AAGTATGCGAAAATTATGTTACTCCAAATGATACTATTCAATGGATAGCA
ATGAATTCTCTTGTTCATTCTGATAAAAAAGTATGGATGCCTTTGCAATT
TGTTACTATGTATACAGAAGAAATGTTTTCTAATGAAAAAAGATATGTAA
CTAGCGCTGTATCAACAGGCACTGCTTGTCATGAAACTGTAGAAAAAAGT
ATAGAAAATGCCCTAATTGAATATCTTCAAATTGATTCCTTTAATTTATG
GTGGTATGGAGGGTTTCGTGCGAGAGATATAGAAATAGATATCACTCGAA
ATATATCAAGCTGGTTCGACAATCAAGTAGCTGTGAAAAAATTTTTATCA
AAGTTTAATGTACATTTTTCAGATGTAAGTTTTGATAAATCAATTTATAT
TGTGCTATGCGAAATAGAGGCAAAAAATTCAAGTGATGCCTTTCCTAAAT
ATACTGTTGGCGTTCAAGGCGGATATTCACTAGATAAATCAATATATCGT
GCATTTATGGAATGTCTAACTGTACTAGAATATAATATGAATGTCACTTG
GACAGATAAAGAGAAATTTCTTTCAGTCACACAGGAAACACGTGTTATAG
ATAATTTGGATGATAATGTTATTTATTATTCAAAGTATGGAAAACCAGAA
TTGCAATATAATACTAATCAATTAAAGAATGATACGGAAAAAGTTACAAA
TCTGAAGGCACTTTTAGAAAAGTTGCCCACAATTAGTCAGTATGCTGCAT
TCTTACCTATCACACCTTCAGAGTTTAGATATATGAATTGTGAAATAAGC
AGGGTGATTCTTCCAGAATTGTTATCCATTCATCTTCCTTCATATCCACC
TTACTACCATGTAAGATATGAAGAAATTGGAGGTGTGGTAAATAATATTC
CACATCCAATTGCATAA
ORF3185 (SEQ ID NO:101)
ATGATCACATTATATTTTCTAATATTTGTGGAAAGGAATGATATTTTTAT
GAAAAATTTTGATATTAGAATAGGAACGCAGACATTTGACAATGATACGG
AAATCATTTTAAAAAGAGGGGTCATTCATAAGAACGAGCTCGTTATCAAT
AAAGAGGAAAGTTCAAAAGAATTTGTTTCAACTTTTAAAGAATTGATCAA
GAAAAAAACTATCGCTATATCTTCAGAAGATGCAATCTACAATGATTTTG
AAACACTTACAAAATTTGGTTTTTTGACAATTTCCAAAAACCAAACACTA
AAACCTCTTTTGGTAGTTGAAGATGCTTTATTTGATGACATGAAGAGCTA
TTTTCAGGAAGAAATTGAGATTTTATCATCCTCTGAATTTCTTTTAAAAA
AAGATATTCGGTTACTTACTGAGAACAAAGATATATTGCAACTTACCAAA
CTAGTAGATGAAAAAAAAGAATTTTTGAAAAATTATAATTATATTTATTT
AATCACAAACATTCCCAATATTTCCTTATTACGTGGATTTAACAAGTTAA
TGAAAGAGACTAACTGCATTAATACCATCGCATTTTTTGATAATGAAAAT
GTCTTTGTTACTTGTATAGAACATGGCGAAACTGGGTGCTATGAGTGTTT
AGAGCAGCAAATACTCTCTCATTTTGATGGTGTTGTTACGGATTATTTAG
TACGGTCAGAAAATAATGTTTCCACAGCTGAATTGATGTTTGTTCTTTCA
ATCATAAAAAAAGAAATCGAAAATACTTCTATTTATGGTCAATCTTCTCT
GTTAGGCAATCTTCTCCACTTTAATTTTAATAATTACGAGTATACGTTTA
ATACAAATAGAATCCAAAGTTGTTGCTCTACCTGTGCTACTTTCAATAAT
ATATTATTTGAAGAGCAAAATATTCGGTCAGTGAATATACTAAAGGAGCT
GATGAGTAGTGATTAG
ORF3222 (SEQ ID NO:102)
CTAGAGGAGGCGTGTTGGATGGAGAAAGAGCAACAAACGGATGATACTTG
GGTTTTAGTTGAAATTGTTAGCCTTATAACCAACGTGGAACGACAAAGAT
TGAGAGAACTTAGTTATACAGAGTTAGAAGAATTTTATGAAAGAGTAGTA
GTGGAGCAATAA
ORF3224 (SEQ ID NO:103)
CCCTATTTTCTAATTAAAGATGTAAATAAGGAGGATTTTAAATCCATGAA
AAAAATAATCGCGTTAATCACAGCAGTTGCACTTCTATCAATGATTGGAT
TTGCCTTTATCCCTGGTACTGTTGCGAATGCAGCGAATTCATCAGAAAGC
AGTTTAACATACAAAGATGTACGCAGTGGATTTTACTTTGTTGGTTATGA
AAATGTACAACTTGAAACAGGAAAAACATACAAGTACACAGTAGCGTATG
AAGCAAATGTTGATATGAAAATGACAGATACTATTACAGGACAATCAGCA
AAAGCAGGACTTTTTACACCAAAATCATCCGGTGCCGAGCTGAACACTAG
TTACGTAAGCCGAACAAAGAATAACGTTATAGACGTAGCTGATGCAGGTA
ATAAGGTTTTTAAACACACATTTGAATTTACAGCGAAGGAAAATACAAAA
GCAGATATCGGTGTTTTCTTAGGAGCAGGTTCTGTTCTTCCAACAACTCC
AGAAACCACGTCCCTTTGGAAAAACGTAACCGTGACAAATGAAACTCCAC
TAGTACAAGCGGAGGCTCCAGTTATTAGTGCAGAAGATAAAACTATCAAT
CAAAATGAAAGTTTTAATCCACTAAATGAAGTCACTGCGTCAGACGAAAA
AGACGGTGATATTACAAAGGACATCCACGTAACCAAAAATACGGTAGATA
CGACTAAGAGTGGCAAATATGATGTGGATTATAGCGTTACAAATTCAAGC
AACTTAACAACGACAAAAAGTATTAAAGTAACCGTAACCCCAGCAGCAAT
AAGCAAAAATACAGCCCCAGTTATTAATGCAAAAGATCAAACAATCAAAG
TTGGAGATGTGTTTAATGTGTTAAAAGGAGTTACTGCGAAAGATAACGAA
GATGGTGATTTAACAGCAAAAATCAAAGTGACCAAGGATACAGTGAATAA
TTCGAAAAAAGGTGTTTATCAAGTTACATATACTGTGACAGATTCTGGCA
ATCTATCAGCTTCACTTACAATTAAAGTGACTGTAACTCAAGATGGAAAA
CTAATAGTAAATCCAAGTGATCCAACAAAGCCATCTATCACAAAAACACC
TGTGGAACCAACAAAAATTGAAAAAGATCCTGAAAATAGAGCGACTTCCG
TTTTGAAAGCAAGTAAAATACCAAAAACTGGTGATAGTTCAATGATATGG
CTAGTATTTGTCGGACTCGGCTTAACAGCTATTGGTATTAGTTCTTATCA
AAAAAAAGCAAATAAATAA
ORF3277 (SEQ ID NO:104)
GTTGGTTACTTACAGAGAAAAAAGATATGTTTCGAACCTCGAAAAAGAAG
GTGCGTTAATATGTGGTTATTTTTTAAACGGGATATATTATGGTTCCTTC
AAGGGAATAAATGGAAAATAATAGGCTTGGGAATGCTTTTGATATTAGCA
ATTTTAATAAATGTTGTTAATGCAAAAAATGCATCGGGGACGATAGCAGA
TGTGTTTTTGAGCTTTTTAAAACAGGATAATGGGGCGGAAAATCCACTCA
CAAGTAGCTTGAATTGGATTATCATTCAAAGTTTGCCAGTTTTTCTATTT
GGAAGTTATTTTTACAAGGAATTGTTTGCATTAGAGGAATTTATTACTAT
TCGCTTTAATAATAGAATGCTGCCCTTTCTTTCCAAAATTTTACTCATAA
TTACGCTGATGTTGATTTATTATTTTGTAATTATTGGGCTAGTAGTGTTT
ATTAGTTTTCTTTTTGGTATTCGTTTTGATGTACAACCGACGCTATTATT
TATAGATTTAAATATGCCACTTTATGAAATGGGTTTGCACTTTTTTGTTG
GAGGGCTGGCACTTATCATGCTTCAATTGTTGCTTTCTATAATAATTAAA
CCATTTTATGCAATTACGGTAGTACTTATCATTATTGTGACCAATTGCTT
TATTACAAACTTTTGGATTATCGGAAGCGTTTCGAATGTTGCTGGATTCG
CTGAGGCAAATAATTGGCTGTTATTAAGTATTCAATTGGTATATATTATT
TTGGTGATGCTCATTGGCGGAAATATTTATCGAAAGACAGATTTATATAA
ACTAAACTAG
ORF3278 (SEQ ID NO:105)
AAAATGAAAAATCATAATTTGGTCCGGTTAAATTTTGATTTTGAAAGAAG
AAAATATTTAATGCTTCTATTGATATTTGTGTTTATAATAGTATCACGCT
TGTTGTACACCAGAAATGTCGAGACAGATGCCCTTTACATTGTACAGAGT
TCCGTATCAGTTGAAGTTCTATTTATAATTTTGAGCCCGTTCTGTTTATG
GATGAATCAAATATTGTGTTTTCAACGCAGGGAACTAGCTGTTGTTCGGA
TTAAGAACAAATATACACTTTGGAAAGTCAATGCAACTGTTATTTTGTGG
AATGCCTTTTTGCTTGCAGTATTAACTAATGCCTTAAATTATGCAAATGG
TGTTATTGTAATGAATTCCCAAATAGTACAAATTTACATTTATTCTTTTA
TATTATTTGGTTTAGGTTTAGTTCTTGTAGGTGTTTTACAGAATATACTA
CTTGTAATTACAAGTAATAAGGCAATTGCTTTTTTTGTAGTATTTCTGGT
ATTTTTCTTTGATACTAGCACAATCAAATTGCAGCTAATTTCCAATCTGT
TTATAGTGAACCCTAATGATCTTACTGACTTGTTGTCATTTGCTGGGCGA
GTTTTCTGCTTGGTTGGTGGGATTATTGTATTATTTTTAATAAGTTGGTT
ACTTACAGAGAAAAAAGATATGTTTCGAACCTCGAAAAAGAAGGTGCGTT
AA
ORF3279 (SEQ ID NO:106)
ATGAGTCGATTTTTAAAAATAGAATTTAACCGAGTTTTTAAAAGTAAATC
ATTTCTTGCTGCTCTTGCACTGGGAGTGTTAATTGTACTAATTCAACAAA
TAACTGTCGCACGTTACTATTCCACGGCAGAAGAAAATGTATTTTTATAT
TTAACAGGTTATGATACAACGGGGCTTGGAACAAATCTTTATTATTTATT
GTTACCATGTTTAGTGGCTTTAGCTGGTGCGGATTTATTGGGTGAAGATC
GGCGTAGTGGCTTAGATATATTTTCGCGAATAAGAGGAAATGATAAGCAG
TACTATTTTTCGAAGAGCATCGTTGCTTTTATTGCTGGAGGGGTAGTATT
TTGTTTGCCTTTAATTATGGAACTTTGTGCATTAATGTTAGTGTATCCCT
CTACACCACTTGATTATTTCGTAGCAGAAGTACCGGTTACATATGGCGCG
ATGTTTTCGAATATATTTTATAACAGTCCGCTTACGTACGAGTTGATATT
CCTTGTAATAGGCTTTGCTTACGGAGGATTGTTTGCTTTGATTGGAATTT
TAGTGTCTTTTTTTAGTTCTAGTAAATATGTAGTTTTATTGAGTCCACTA
GCTATTTATTATGGTGTGTGGATAGTGTTTTCTTTGATTGGCTATCCAGA
GTTTAGTCCGTTTGGTTTCTTAACTCCAAAGCAAGGTTACCCCTTAAATT
TCTATATTATTTGGGTAGAATTTCTACTTCTTTTAGTAGTGATAATTATG
GGTATTATTTGGAGGGTGAAAAATGAAAAATCATAA
ORF3283 (SEQ ID NO:107)
ACCAAACTTCTGGAACTGTTAAGACAAAGTATAGTATATACAGCAAAAAA
AAATAATGATAGGTATGTATTAATGTATAAAGATGGATATACATCAAGAA
ATAATACATACTACGGGGAATTTTATGGTCACTATGCAGGCGGAAAAGAT
AAATTTTCATATAAATTAGAAAAGAAAAATAATAAAAATGTAAAATCACA
ACTTATTGTAGATTTATTCGTAATTTAA
ORF3291 (SEQ ID NO:108)
AGAAGAGGTGTAAGGAAAATGCTAACAAAAGAAATATTACCTATTGGAAG
CGTTGTTTATTTAAAAGAAAGCCTGAAGAAAGTTATGATTACGAGTAGAT
TAATTACGATTCAGGGAGATGAAGAAAAAGAATTTTATGATTATGGAGGT
GTAGTTTATCCAGAGGGGACAAAGGACGACAATATTTTAGCATTTAATGC
AGAAGATATAACAGATGTGAAGTTTAGAGGTTTTGTAGATGATGATGAGG
TAGTCTTAGTGAAGAGAATGAGAGAATGGCAGAAAGAAGAATTTGGCGTT
TCTCCAGAAGAGGAGGATGAGATACTTGAATTATGA
ORF3316 (SEQ ID NO:109)
ATGAGATATGAAGCAAAAATAATTTCAGAAAATCCTAATATTGAAGAAGA
GTTGAAAATTAAAATAAAAGATATTGAACTTTTATGTTTTGTTGAAGAAT
ACAAATGTTCAGTAGAGGTTGGCCAAGAATATATTGTTGAGTTAGAGACG
GTCGTTTTCGATGATTTAGATATTGAGAAAAGTACTTTACAAGCTAAGGA
AATAACTCAAGTAGCAGATAGTTTTGCATACTTTATCAGAGGCGTTTTTC
ATCCTTCTAGTAAGAAAATAGATTCAGGGATAGAGATTGATTTAGCAGAT
GAGGATATTTCTGATTTTTGGTATTTGGAAAATCAATTTGTTGCATTAAA
TGTTGATAGATTCAATATAGATGTTATAGAAAAAGTAAATAAGTAA
ORF3324 (SEQ ID NO:110)
GAAGTGATAGGTATGGCTCGAATCGAATATGAAGAAAAAATTCAAAAAAG
CCTACTGGTTTTATACTTTAGGGGGTCTACCATACAGTCTATCTGCAGGG
AGTATGGTATTCCTCGTTACGAATTTCACAAATGGATGAAGTTACATGAT
GCCGATAAACTGGAAACCAAAGAGGTGAAGACTTTCCTACAGATAAGAGA
ATTAAAACAACAAAAAAACAAATTAGAAGAAGAAATTTTGTTTTTAAATG
AAGCAATCAATCTGTTGGAAAGTCCTTGA
ORF3325 (SEQ ID NO:111)
GAAATTACGAAAGGAGTTGTGCATTTCTTGAAAAGGAAATTAGTTCTGGC
AATGGTACTAATCAGCTTTTGTGGGATGTTCCTCTTATCGCCTGAACGAG
TTCAAGCTAGTCAAACAGTGGTTGAGGTGTTCATTTCAGAGGATGATTTG
GATGTCAGTGAAGAAAACATACAGGAAATCCCTAAAAAAGAACTGGCAGC
CTCTTCCGATAAGGATAACAAACAACCCATATTACCTAAAGCAGGTGATA
CAGTAACTAACTATACCTTGTTGGGTTTAGTGCTCGTCCTTATTTGGTTG
ATAATGCAAAGAAGGAGTAAGAAGAAAGAATAA
ORF3326 (SEQ ID NO:112)
TTGTCGTCTAGACAAAGGAGGAAATTTTTTATGAATAAATTAGTATTAAG
TACGTTAAGTGTAGCAGCAATGGGTATGGTGTTTTTTAGCGGAGGGACAG
CCTACGCGGCAGATAAAQAAGGAAATACAGTAGTGGAATATTCTGTTGAA
GGGGATTATACCCTCGTTGTACCTGAAAAAGTAAATTTATCTAATGATAA
CGCCACAGAAATGTCTGTGAAAACAATCAATCGTAACTTAGAACCAGGAA
AAGAAGTAGAGGTGACATTATCTAGTGGATTATCAGCCGATGGAGAGATT
GAATTACAACGGGTTGGCGCTATTTCGGACGTAATAACCAGTAGTTTTAA
GAGTAATAATAGTGTAGTACCCATGGCTAATCCCGTCATTGGAAGCTTTT
CAGGATATGCGATGGAGGAAACAGAAGTAAGTAAAATTCAAATTGGAAAC
CCTCAAGGAGACAAAAAAGCTGGAGCATATCAAACAACGTTAACTTTTAC
AGCTGCTTTTAAATAA
ORF3327 (SEQ ID NO:113)
AAACTAATTAGGAAAGAAGGAGGGGAGCTGATGAAGAAGTTTTCCATGCG
AGTGGTGCTTATTATTTCTGTGCTTTTTATTGCTTTTGGGAGCGCTAATG
TTTCTATCGCACAAGAGATGGATACAACGAATAAGCTTCCAGAAGAGGAG
CTGGGTTCTCTGGATACCTCTAATCTTATTGCAGAAGAAGTCGCGCAAGA
CAAACCAGCAGAAGTGGATAATCTAGAAGAAATCCCAACTACGGATGAGT
TGATGCAAAATCCAGAGGTTCTTGAACAGCCTGTTGTTGATTCCGATGAT
CCGGATTTAACAGTGGTAAGCTCAGGTGATTTTTGGACGCTTTATTATAA
TTCTGCGAACGATGAATATAGCATGCGTATGTTTGGCAACGTGCCAAGCA
GTAAACCAACCGCTTGGAATAGTTATTTGAAATACATTAAACATATAGAG
ATTGAAGAAGCTACTCTGACAGGAAGTTTTGCATCGTATTTTGACAATAG
CGCTTTTCCAGCACTGGAAAGCGTGAGGATTGAACAGTGTAATTTGTCTG
GAGTGACGTCTTTTAGAACCGCATTTAATAATCACTTAACACTTGAAAAA
GTTATTATCAAAGACAATGATTATCCCACAACATCATCTTTACTTACTAC
GGAATATATGTTTTCTCATGCTATCAAACTTACGGAGCTTGATGTGAGTG
GTCTTGATACGAGTGCTGTAACTAATATGAAGAACATGTTTGGTGGCTGT
AACTCACTTGAGGAACTGGATTTAAGTAATTTTGATACGAGTTCCGTTAC
TAATATGTCAGGCATGTTTGGATACTGTGAATCACTTGAGAAATTGAATG
TAAGTCATCTTGATACGAGTTCAGTGACTGATATGAATGCCATGTTTTAT
GGTTGTACTAGTCTTGAAGCGTTAGATGTAAGTAATTTTGATACGAGTTC
AGTGACTGATATGCGTGCCATGTTTGCTGATAATGAAAAACTGGAGAAGC
TTGATTTAAGTACCTTTGATACCAGTTCAGTGACTAACATGGGTACCATG
TTTAAAGATTGTACAGCGTTAAAATCTTTATATCTGGATAATTTTACGGA
TGCAGCAATTATGACAGATATGTTTAAGGGAACCACCTCCTTAACTTACT
TGTTTGTCAGTCACAATTTAAGTACTTTTACTAGCTTAGAAAATACAAGC
TGGTATGACGAAAAGAATTGGGTGCAGTTTTCTAATCTCTCGCAACTACA
GACATATCACCGGAAACAAAGTGAACCTACAGGCTACAGAAAAGGCGCTT
TCCTTTCTCTAACAATGGATGCGATGGGTGGAGAATTTGAAGACGCGGAG
GAACAAAAAGTACAAAGTAAAATTTCTGGGGAGTACTGGGAAGAAGTGAT
TCCAGTAAAAGAAGGACATTACTTTGACGGGTGGTATCTTGATCAAAATT
TCACTAATAAGTTTGATTTTTCTTTACCAGCAACTGTTTCAGCAACCCTC
TACGCTAAATGGGTAGAAAATTATACGGTGGTTATTCCAGCTTCTATTTC
CTTAAATGAAGCTACTGAATTGAAGGTAGAAGGGATTAATCGAGGGGATA
AAAATTTGTCCGTGGGATTAAACCGAACAGCAACGTCTGTTTCTGAGAGC
AACAAGCTAACTTTGGCCAATACAGCAGACACAACAATCCAATGTTTGGC
ACCACTAAGTTGGGACGGTTCCGAAAACAATCCAAAAAATGCCATTTTAA
CCCTAGCACCAGGTTCGGAAATAACAGAAGGCGAGGCAGTCCTGGCTATT
GAAGCACCAGAGAATATCCAGGCAGGAACGTATACAGGGAATCTGGTATT
TTCGATTAATTATGAATAA
ORF3361 (SEQ ID NO:114)
TCCTGTTTCAATCAAATAAAGGAGCGTATTTTAATGAAAGAATTAAGACT
GTTTTTAGATTATAAATGTTATCCTGTTTGGGTATATAACGATGCGGGGA
TTTTAAAGGAAAATGATTTGCCGGATGAATTGAAACAAGATGAATATGAT
AAATTGTTCATAGATACTGAAATTGAATTTCGATATGAAGCTTTTAAAGA
TGAGAAAGAAAAGGAAGAATTTTTTCATGAGTTCATAGAAGTTCAAAAGC
ATTTGAAAGAAACATTAGGTAAGGAATACAATATCGTTAATAAGATTTTA
GTTTAG
ORF3362 (SEQ ID NO:115)
AAGGAAGGTAATAATATGAAAAAAATTATGAAAAAAGATGATTATTCTAA
AATGCCATGGGTATCAGCGGAGGATTTATATCTTTTATTTGAACAAGCAC
TCAAGGACTTTAAGCAAAGCAAATTAAGTAAAAAAGAGTTTTTTGATATT
CTTGATGAATTGACTATGAGGCAAGTAGATACCTATGAAATTTTAAAAGA
ACCTTTGAGAGGACAGCTCGACAATGAGTTATATAATTTATGGAACACAG
AAAATTATGATGATGTAGATATTATTACATCATTGTTAATTAATTTAGGC
TTGAAAAATACTTATAATAAAATGAAAAAATCTATTGAGGATACTTCGGA
AATATCACCAGAAATACTTGAGGAAATACAAGATGCAATTGAAGAAGTCG
GAGATAATATAGATGATCCATATCAAGATTATATGAAGAAAATGTAG
ORF3365 (SEQ ID NO:116)
GAGGAGATACATTTGTTTAATGAAAAAGTAAAAGAAATGATCCGAGAATT
GGATAAATATAAATTACAAGATAAATGGGTTCGCTTTGAAAAACTTTCTA
AAAACAAGAAAATTGATAGTGAAACTTTTCAAGATGAATTTATTATTATA
ACAAATGGAATTCTTCATGTGGAAAATAAAAAATTCCAGATACTTCATTT
TTTTTCTAATGGAGATATAATTAACCAACAAGTTGCCAAAATTAGCGGAG
AAAATGAATTAAGATTAGTTTGTGATACAGATGTTTCCTTAATTTTTATT
GATAGGGAATATTTCTTAAACTATGCTACCAATAAGCCTTCTTATATGGA
GTGGCTTTTAGAAGAAACATTAATAAACAATAAGAACCTTTATAATGAGT
TAATTAAGTATGACTTGTCCGCAGAAGAAAGAATAGTCTATGCCCTCCAG
TACCTATGTGACAAATTAGAAATTGAAAGTGAAAATGGCTACCAACAAAT
ACCTAAATATATTAATAAGATAAAAATGGCGAAATACGGCAAAATATCTC
GCAAACAACTAAACGAAAAAATAATACTTCTCCTCGATAAGGAAGTATTA
AAAGAAAAAAAAGGAAGATTCTATATAAAAAAATCAGCATAA
ORF3378 (SEQ ID NO:117)
CATCTTTTTTTTGCTATATTAAAGGATAGTAACGTCTCATATAGGGAGTG
GAGAAAAAGAGTGTTAAATAAAAAAAGAGTTATAGTTGGGGTTTTGCTAC
TATTACTAAGTTTAATTTCTATTAGCTATTATTCAGAAACCTTTAAACTA
ACTTTAAGCTGGTTGTTATTTGCAGTAGTTTTAACGGTTCTTTATTTTAG
ACAAGAGAAAAACTTTCAATTTCAATGGTCAACATTGATAACCTCACTAA
TAATTAGTTTATTTTGGATGGCTTCATCATTCAATGGTGGACCATATGGC
GGGAATTTTGTTTTTAATAGTATTATTTTAGCAGGAACTTTTCTTGTAAT
CACTATCTTTGTTTTACTTTTATTACTTGAAATGCGAACGGAATATAAAG
CAAGACCGAATCGCAAAGTAAAATGGCCATTTTTCGCACTATTTACGAGT
ATTCCATTCGTGGTCTGGATGATTTCATTTCTAGCCTATTATCCAGCAAA
AATGACATTTGACTCCTATTACCAATGGGGAATGGCTCACGGTATTCGCC
AATATAGTCAGTGGCATCCGCTTTTACACACTTTGTGGATAGAAACAACA
AGTGCGATTTACGACTCACCTTCGAGTTACATTTTTTCTCAAATAATTGT
TGTTTCATTAATCGTTGGCTTTGCTATTTATACTCTTGTAAAAATGGGCG
CGCATATTTGGATTGGTGTTTGTATTTCAATCGGCTATGCCATTTACCCT
GCAGCAATGTTTTATTCTGCAACAGCATGGAAAGATTTTCCATTTGCAGC
CTTTATATTACTTTTCACCGTTTTAATTTTAAAAATAGTACAATCTAATG
GAATGTGGCTGAAAAATTGGTGGCACCTTATCGCTTTTGTTTTAGTAGCT
TTTGTTTGTATAAATTTACGAAACAATGGAATGATGATTATCATCGTATC
GCTTCTGTGCTTGCTTATTTTCATGAAAAACTTTCGTCTTATTATTACCG
GTATTCTTGTTGGAACCTTGGGACTGAATTTTTTATTTGGTCTGGTTATG
ACAAACGGGCTTAATGCGCAACCTAATCCATTAAACCAAGCGCTAGCAAT
TCCTTCCCAACAAATTGGGGCTACTTTTTACAATGATGGAAACTTTACTC
CTGAATTAAAAGAGTATTTCACTTCCATATTACCTGAAGAAAATTGGAAA
AAAGATTACAACCCTTATACTGTAGACCCAATTAAGCATGATACCAAATA
CAATTCATCCGTCATTGAAGATGATTTTGGACTATACATTAAAAATTGGT
TCAAACTCTTAACGGCTAATTTCGGTACTTATGTAGGGGCTTATTTAGAT
CAAACAGCAGTCATTTGGCAATTCTATTCTCCAGAAAATTATAAAGTATT
CTTTGATACTTCAGCGAATATTCAAGATACAAGATATGATGTGAGAGCAT
TCGCCAAATTCTTCCCAGAAGGTTTATCGGAAGAAGAGATTAATAAATTA
GGATATGAAGTCTATCAAAATGAATACAAAAATGCAACTGGAAAAGATGC
TGTTAGCTACAATGAGTATAAGAGACGGATTGATGACTCTACTAATCCAC
TTATTTCAATATCTAAAGCTCCAAGTCTGAAGAAAATAACAGATAGCATT
TATGCAAAAACAACAAATGAGTGGCAAAATTATTTATTAAAAGGAGCCAT
TCCATTAGTATTGCTCATAATAGCAATTGCTGCCGTCTGCCTCCAACGTC
CTAAAAAGAAACTTCTTATTTTTGCACCTGTAGTAATGGCACTTATTACT
ATAGCAATCGCAATGCCAGCAACAGACTTTAGATATTCTTATAGTTTTAT
TTTCACCGTGCCTATTGTCTTTTTTGCAACTAAATTAAAAAATTACAAAG
AAAATCAATTTTAA
ORF3379 (SEQ ID NO:118)
AAAATTACAAAGAAAATCAATTTTAAGGAGAGAATAATGGGAATTCTAAA
TGAGAAAGTAGCTGTACTCTTGCCTTGTTATAACGAGGAGCTTACAATTG
GTAAGGTAATTGATGATTTTAAGAAAGAATTACCAAATGCGGATATTTAT
GTGTACGACAATAATTCTAAAGATAAAACCTTTGAAATAGCGAAAGATCA
TGGTGCTATCGTTCGAAAAGAAATGCGCCAAGGTAAAGGTAATGTAGTAC
GTTCTATGTTCGCGGATATAGATGCTGATTACTATTTAATGGTCGATGGT
GACGATACCTATCCAGCAGAATACTGCCATGAAATATTAGAGGTGCTTCG
CAATAAGGAAGCTAATATGGTTATTGGTGATCGTCTGAGTAATGGTACCT
ACACTGAAGAAAATAAAAGAAATTTTCATGACTTTGGTAACTCACTAGTA
CGTAATACAATTAATCGTATCTTCAAAAGTAATTTGAGAGATATCATGAC
AGGCTACCGTGGCTTTGATCGTTATTTTGTTAAGACTATGCCAGTTTTAA
GCCCTGGTTTTGAGATTGAAACTGAGATGAGCATTCACGCATTGGAAAAT
CGCTTTTTAGTGAAAGAAATTGAAATTGATTACCGTGATCGTCCAGAAGG
TAGTGAATCAAAACTAAACACTTTTTCTGATGGTTTCAAAGTAATTATGA
CGATTGTAAGATTATTTAAAAATAGTCGTCCGTTTTTATTTTTCAATTTA
TTAGCCTCTTTGTTTGTGCTTGTAGGAGTTCTAGTTGGTTTGCCAGTCAT
AATTCAGTTTGCTCAAATTGGCTTGGTACTAAAATTTCCGAGTGCATTAC
TTGCAACTGGTTTAATCATAATGGGTATGCTGTTCTTCATTTGTGGATTA
ATCCTTGATACGATAGCTCATAGAAGCAGACAAAGCTACTTCTTAGAACT
TGTTAAATACCGCGAAAGAAATCCATTGAACTAA
ORF3386 (SEQ ID NO:119)
AGTATGAAAAAGTTACTTTTGTCCATCAGTATTATTGTCGGTTTAAGTTT
GTTGGCTAGTTGTGGTTTAGAAAAGGAGTACAAGCCAGATATAAAAGTAA
ACGAGAAAAATGTTTCTTTAAAAGTTTGGGTGGATTTAAATCAGGGAGAT
TTTTATAGGAAAGTGGTAGAAGATTTTAAAAAAGAACATCCTGATAAAGA
TTATGATATTACGGTTATTGAATCTGAATCTGGACGAGCACAAGAATATG
TTCAGAAAGATCCAGAAGCTGCGGCGGATGTATTTATAACTCCGAATGAC
CGTCTAGGACAACTGGTAGAGTCTGGTGCGGTTTACCAATTGACAAAGTA
TACAGATGATATTAAGAAAAATAATACACCAACCTCCATTCAAGCTGCTA
CATATCAAGATAAAATGTATGGTTTCCCTGTGACGGCGGAAGCGATGTTT
ATGTATTATGATAAGCGCGTTTTTTCAGAGGATGATATTAAAACTTTTTC
TGGAATAACGTCTAAAGGGAAGCTCGGAATAAATCTTGCGGAAGCTGGCG
CGGATTATCGTGAAACACCTTGGTTTATTGCAAATGGTACATATCTTTAT
GGTGAGAACGGCGAAGATCCTTATGGGACAACCTTTAACACTCCAGAAGG
CGTCCAAGTATTGAACTGGATTGGTGAGCTGAAAAATAATCCTAATATTG
TCGCTGTAAATGCAGATGAGATTAGTGCCCTTAGATCCGGTAAAATAAAT
GCTGTTTTTAGTGGGGTTTGGAATAAAGATGCGATACGAGAGGTTTTGGG
TGAAAATATGGGAGTGGCAGTATATCCAAAGGCCGATTTTGGAAGTGGTC
AGGTAGATATGATGGCTTTCCAAGGAAGCGGGATATACTGTGTGAACGCA
TTTACTAAATCGCCATTAGATGCGATGGAACTAGCTGATTATATTACTAA
TGCGGATGTGCAGGAAAAAGCTTTTAAAGAGTTAGGGAAAATTCCGAGCA
ATTTAGAGGCACGAACTAGTTCTACAGTAGAAAAGGACGATGTGGCGAAA
GCAGTTATTGATATGACATCCGGAAAGCACTCCGTTTTAATGCCGAAAAT
ACCTGAAATGAACGTTTTTTGGCAACATATGAACCCGCTATTAGTAGATA
CTTATAAAGGCAAGATTAAGAAAGAAGATTATCCAGAGGCGCTCGATAAA
TTAGTCAAAGATATTACACCAGCGAAATAG
ORF3401 (SEQ ID NO:120)
AATCAGCGAGAGATAGGTGTAAAGAGAGTGAAGAATAGGTTAGCTTATAT
TTTTAATGCGTTTTTTATACTTATTTTTGGTTACTTACTTTGCATAAGCA
TTTTTAAACCGCTAGAAATTAGTTTTAATCATCCGAGTATATTTATATTA
TTTAGTGCTGCGGCGCTTTTAGTACTTATAGGATTCTATCAATTTTCTAC
TAGATTGAATACAAAAGGAGATGGTGTAATAACCATCTTTTTGGTTAGCT
TGATTATATTAACTCAAATTTATTTGCTTTTCTCCTTGCAAATGAATTCA
TATGCAGATGCTTTTCTTATCAAGGGAGAAGCATTGAATATGCTTTCTAA
TGGAGGGCATGCAACAACGCAGAATTATTTTTTAATGTATCCTAATAATA
TATTTATAACAATTATTCGATACTGGCTATATTCTGTTGGAGGGACGCTT
GGTATTACTAATACGTATTTATTAGAAAGTGCTTTCCTTTTTGTTTGTAT
GAATATTACTATTTTTGTATTGTACTGGATTGTTCGTAAAGAAAATGGTA
ATAAATTTGGGAACATCTACTTATTAATTGTTTTATTCTGTGTGCCATTA
TTTGGTTATATTTGGTATTTCTATACAGATACACTTGTACTGCCATTTAC
AGCGCTGATTGCCTTATTTTATTATCTATACACGAAAAGTAGTAAATGGT
GGTATTTCATCATTATTGGGCTGCTGTTTGCAGTAGGTTATCAAATTAAG
CCGAATATCATCATTTTACTTCCAGCAATGCTTATTCATTTATGTTTTAT
AAGAAATTGGCGTAAAATTCTATTGAACACGGTCATCGTAGCAATTTGCT
TTTTCGGTTTAAGCACTGTTTTTACACCAATCGCAGAGAGTTATGACTTT
AAGAAAGACCCAACCATTGAATTTCCTCAAACACATTGGATTATGATGGG
GCTTGGCGATCCAGCCGGTCGTTATAACAGCAATGATGTTGCTTATACTT
CACAATTTAAAACAAAAGAAGAAAAAGAAGAAGCCAATATTGAAAAAATT
AAAGAACGTATTGAAGAACATGGACCGCTTGGCTTAATAAAGCTTTTTGA
TAATAAGGTGTTAAATACTTGGACAGACGGAACGCGAGCATATACGTGGT
ATGTTAATGCTGCACTTGATTATCCAGCTCCTTATGATTACTTCTTTGGA
GATAAACGGGTTGTCACTGAATTACCTGCTCAGCTGTTCCATATTATTAA
TTTGTTTTTAATTTGTCTAGGTGCTTTACGTTTTTATAAGAAAAGGGAAT
TTGATATGTCCTTTTTCGTTAATATTTCGCTAGTAGGTGTTTGGCTATTC
CACTTATTCTGGGAAGCAAATCAGCGTTATATTATGTTTATTACGCCATT
AATGATCTTGTCCTCTATATATGGGTTTAAATTTATAGTAGAATCTTTAT
ATACGAAAAAATTTGATTTGAAAAAAGGGCTAAGAAAAGGCTTTTTAATC
GCGAGTTTCTGCGTATTTTTATTAAGTACAGTTGCTTTTGCTTTTATTGG
AAATTCGGTAGCAGGCGAGTCACAGGATATCAATAAATATCTTGTAAAAC
AAAGTTATGCGCATATAGATCTTCCTGTTACTAGCAAACAAATTGTTAAG
CAAACGTTTAATGTAGATTCGCCGTTCAATTCTATCCAAATAGCGGTTCT
AAAAGAGCCAGATGAAGCTAGTAAATATCGTTTGAAAGTTGTAGATAAGA
CAAATAAGAAGGATATTTACGATGAAGTTATAGCTGGATCAGATTTTGTA
GAAGCTACAAATTACCAAATTAATGTAAATGAAAAGCCAAAAGGTAAAAC
TGAATATGTTATTGAAGTCTATCAAGTGGAAAATAAAAACCCTGAAAAAC
CATTAGTTTTAGGTACCTATACTCCAGATGCAGTAGATCTTTATCCGTAT
GGAGCACTGTATGTTAATGGTGTTAAAAAAGAGAAGCAAGATATGGGCTT
CACTGTCTCACATGTTGCTTCTGAACCAATAATACCGAAATATGTCTCCG
CTATTTTTGATTTGGGTGTTATAATTATTTTTGCAGGAACATATTATGTG
TTTAGAAGGAAAACTGGAGATAATAGATGA
ORF3402 (SEQ ID NO:121)
GACAATAAACAAATTAAGGAGTGGTTGGAGAATATGATAAATAAAAAGTG
GATGAAAATTGTAATGATTCCGATGCTAGTTGTTCCAATGTACGGTTTGA
CAACTGTTGGCGGACAATTACAAGATTCATTAACTGGAGAAAATTCCTTT
GTTAAAGAGGTTGAAGCTGCAACGACAGCATCGCAACAAGCATTTATCGA
CAAAATAGCACCTGCTGCCCAGGCATCTCAAGAAAAATATCATCTGTTAT
CTAGTATAACTTTAGCTCAAGCAATTCTAGAATCTGGTTGGGGAAAAAGT
GGACTTGCTACACAAGGATATAATTTATTTGGTATAAAAGGGAAATATAA
TGGACAATCAGTTATCATGACAACTTCTGAATATGTGAACGGTGAGTGGA
TTAAAATTGATGCTGAATTCCGCAAATACCCTAGCTGGAATGAATCTGTC
ACTGACCATACTCTTTTATTAGTGAACGGAACTTCTTGGAATAAAGACTT
ATATAAGAAAGTTGTCGACGCAACGGATTATAAAGTAGCTGCAATGGAGC
TTCAAAAAGCTGGATATGCAACCTCTCCTACATATGGTGCTAGCTTAATT
CAAGTAATTGAGAATTATGATTTAGCCAAATATGATGTTTTATACGACAA
AATTCTTACTCAAAAATCCACTTCCGGAAAAGCAACTGTTACAAGTCCGA
CTGGAAATGGTGTATGGACTTTACCGTATAAAGTAAAAGGAGTGCAATCT
GTTAGTCCAGCTAGCACATACGCTAACAAGGATATCGATTTAGTATCTGT
TGCTACAACAAAGAGAGGTACGTACTATCAATTTAAATATAATGGTAAAG
TAGTTGGTTGGGTAGATGGCAAAGCATTAACTATTTATGATAGTGTCAAT
TATGATAAAGTAAATGTCGGACGTGCTAAAATTACTAGCCCAGTAAGTAA
CGGTATCTGGTCTAAACCATACAATGTTTATGGAAGAGAATTTGTTACGA
ATGCAACAACTTACGCACAACAAGAAATTAAACTTTTACGCGAAGCACAA
ACTGCTAAAGGTACTTATTACCAATTTAGCATAAATAATAAAACTATTGG
TTGGATTGATAAACGAGCTCTCACTATCTATCCGTATGATTCCATTATTT
CAAGTAAAAATGTGAACCTTGACGGACAAATTACTAATCCAACCGGAAAT
GGTATTTGGACTAAAGCGTACAAACTTGAAGGAACAACTTCTGTGGCGCA
GGCTACGAAATATGCAAATAAAGATGTGAAAATCAGCCAACAAATCGAAA
CTCAACATGGTACTTATTACAATATCAGTATCGATGGGAAAGCAATTGGT
TGGTTAGATAGAAACGCTATTACACTGTATGATCAAGAGGAATACAATAA
AACAGTTGCTATTGACGCAGTAGTAAAAAATGTGAAGGGTAATGCTGTAT
GGACAGAACCTTACCGTACAGTTGGTACAAAATTAATCGGACCAGCGGAA
ACTTACTTGAATAAAGAAGTGGAAGTCGTCCGTGAAGCAAAAACGCCAAA
AGGAACTTACTACCAATTTAAATCTGGTGGCAAAGTAATCGGCTGGTTAG
ATAAAAAAGCTTTCGATGTATATGACAATATTAATTACAACAAAGCGGTT
AATTTAGACGCTGTAGTGGAAAATGTGACAGGTAATGCAGTTTGGACGGC
TCCTTATAAGAGTAAAGGTGTTAAACTTGTTACTTCAGCAGCAACCTATA
AAGGCAAGGCAACAAAAATAACTCGTGAAGCGCAAACAAGTAGAGGAACA
TATTACGAGTTTAGTGTTGATGGTAAAGTCATTGGCTGGTTAGATAAAAA
AGCTTTCGATGTATATGACAATATTAATTACAACAAAGCGGTTAACTTAG
ATGCTGTAGTGGAAAATGTGACAGGCAACGCAGTTTGGACTGCTCCATAT
AAGAGTAAGGGTGTTAAATTAGTTACTTCAGCAGCCACATATAAAGATAA
AGCAACTAAAATAACTCGAGAAGCGCAAACAAGTAGAGGAACTTACTACG
AATTTAGCGTAAACGGCAAAGTAATCGGTTGGTTAGATAAAAAAGCTTTT
GATGTATATGATTCTATTGAGTACAATAAAGCGATTAATATGACTGGATT
ACTTAGCAACGCGCCAGGTAATGGCATTTGGACAGAGCCGTATAGAGTTA
TTGGCACAAAAAATGTAGGACAAGCAACTGCTTATGCTAACAAGACAGTA
CAGTTGATACGCGAGGCTAAGACTACACGTGCAACTTACTATCAAATGAG
TGTAAATGGTAAAATAGTTGGTTGGGTAGATAAACGAGCTTTTACAAACG
TTAAATAG
ORF3410 (SEQ ID NO:122)
GTAGAAAAGGTGGTTAGTATGATGAAAAAACGTATAATTATTCTTGCAGT
ATTAGTGGTACTTCTTATCGGAGGAGTTGTAATCGGTGTTTATGCAAGCG
GAAACTCTGCAAAAGACAACAATGAAAGCAAAACAACAGCTAAAAAAGCC
ACATCTACACCTAAGAAAGCAATTGACACAGAGAAAAAAGAAACAACAAC
CAAAGAAGCGACAACAAAAGATTCCGTAACGGATGATAAAGGTGTAGTCA
CAAAAGGAAGCTCAGATGTAGAGAAAAACGCACCGGCCAAAAATAATAGT
AGTGCAACAGATAAAAGCAATAGTCCAACTACACCGGCTTTTTCGTTATC
TAGTACAGGCTTCAAAACTTCCAATGTATCTTCTGTTCTTGGTGGAACTG
TAACAACAACGTATTTGTCGAGTGCCCCATCATTCGAAAAAATCTTTGAA
AATTTAACGATTGAAGTGAATCAATATAAAGTAGAGCATGTTGTTGGTGC
GAATAAAGCAGTTAGTGCAAGTAATCCAGAAAGCTATTTAGCAAATAAAA
ACGGCTATGTAATCACTTTAGACATTTCCATTAAAAATACTTCTGCAAAA
GATAAAATGTACAAAGCAGACCAAATTACACTTATAGGAGCAAATGAATT
TGTAGGAGGAAGCTTAGATAATTTTGTTCCTTCTAATTTTCATCTTATAG
GAAGTAAAGCAGATCCAAATATTTTCACCGCTGGAAAAACAGCTCGTGGG
CTTCTTACATTTACAATGACAGAAGCTGTGTACAATGATTTAGCAGCTGA
CTCAAAAATAGGTGTTCCAAATCCTGATAAATTTGATGCAAGTGTTTCTG
AAGCTAATGCAGGCGATGATGTAGTTGCCTCATTCCCAGTAAAATAA
ORF3457 (SEQ ID NO:123)
AACGGGGTGTCAACGACGAAAAACAATATAGATTACATTAAAACAGTACA
AAACATAAAAAGTTTTTTTGATGAATTTCAATATTTGGTTTTTCTGCTAG
GTTCTAAAAATAAAATAAAGCTCAATACAGACGGATTAATAGAAATAAAA
GTGTTAACAGGCAATAAAATAAGTTTGACTCCTATAGGTCACTTAGTTCA
ATTTTACTTGGGAATATTAAATGACATGAAAGCACTACATCGCTTTATTT
TAATTAAATGTTATATTGAGATAAACAAAAAGATATTTTAA
ORF3501 (SEQ ID NO:124)
ACTTTCCCTCCCAAGAAAGCCAGTGGGGCTGATATAGCTAAAAGTAAAGG
TGGCGTTACTTTAGAATCAACAATAGGAAATAAAAATATTGAAATGCCAG
ACTGGGATTTTAATAATCCTGAGTCAATGAAAGCATGGGATTTGGCTTCT
GGTTCTTATGCAGAACAAGTTTCTGGTGAAGTTCGTGCAGTAGTTGGTTC
GGATTTGAGAAAAGGAAATATTTGGGAAAATGTTGAGTTACCTAGATTAA
AAAATAATCCTAACGTAACGAAAATAACAACGATTGATCCTAAAACAGGA
CTTGAAAAAATAATTTTTGAAAGGAAGTAA
ORF3593 (SEQ ID NO:125)
GGAGTAGTGACAATGAAACTAATAATGAATGCAGATGATTTTGGGTTCAC
AAGAGCAATCAATTACGGAATCTTTGATGCGCACAATTTAGGCGTTTTGA
CTTCCACGACATTAATGGTAACAATGCCTGCTTTTGAACATGCTGTAGAC
TTATCTAAGCAAACACCCACACTTGGAATAGGCTTACATCTCAATTTAAC
GTTAGGTAAGCCGTTGACAAATGGCGCATCTTTAGTGAACCAAGCTGGTG
AATTAATAAAGCCAAAATTTATTACGCCTGAGTATCCCTATAATGAGGAA
GAAGTGTATCAGGAATTTAAAGCGCAATATCATCGCTTTGTCACATTTAT
GAAAAAGAAACCGAGTCATTTGGATAGCCATCTCTTTTCTACAGATATCT
ATCCGGTTGCGGCTAGTGCTGCGAAACGTCTTGCAGAAGAGGTTGAAATT
CCACTTCGAAATCATGATACAGTGGGCTTTCAGCATGTCGAGTTTATTTG
GGAAAAACCGCTTGAAATTCCATATGGTGCGTATGAGAACCTAGATTATA
TTTATGACTATGCAGAATCGATTTTATGTTATGATTATGTTGAAATCATG
ACGCATCCTGGCTATCTCGATACGTTTATTCTGGAAAATTCAACATTCTC
AACGCCACGCGCGAATGAATTAGAAAGTTTAATTTCTCCTAGAATGCGCC
AGTTTTTAAACGAAAACAATGTAGAATTAATTTCCTATCATGATATTCCA
AAAAAATGA
ORF3594 (SEQ ID NO:126)
GACGGAGGGAAACTAATGGAAGGCACTGAATTACAGTCATTTAAAATAAT
ATCAAGTGCAGGAGATGCCTCTTCCTCGTTTCTAAAAGCAATTCGTCACG
CAGAAAAAAGTGAATTTGCGGAAGCAGAAGCATGTATTGAACAAGCAAAT
CAATCTTTAAGAGAAGCGCATCATGTGCAAACATCCTTGATTCAAGAGGA
AGCGGGCGGGGACTCAAAAGAAGTATCCTTATTGCTTATTCATGCGCAAG
ATCATCTGATGAATGCTTTTGTTTACTGCGATTTAGTTAAGTCAGTACTT
AACTTGTATAAGCGTTTGGATGAAAAGTAA
ORF3745 (SEQ ID NO:127)
AAGGTGATTAACTCATCTATTTATTATTTTAGGAGGTTTTTTATGAAAGC
AAAAAAGAGAAAAGAACAAACATTTCATGAGTATTTTAAAGAATGGGTAG
ATCTTTATAAAGTTGGAGCGATACGTTCAATTACCCTGCAAAAATACTAT
GTAACAGAACAAAAAATTCAAGAACTTGTACCAGAATTAAAAATAAAGGA
CCTAGACCGTTATACTTATCAGCAGCTGCTAAATAATTATGCACTTACTC
ATGAGAAACAAACAACTATGGATTTCCATCATCACCTGAAGGGCGCTATT
TTAGATGCGGTAGATGAGGGGGTTCTAAATCAAAACCCCACAAGAAAAAT
TGTGATTAAAGGAAAAAATCCTCGACCTAAAAAAGCAAAATTTTTAAATC
AATTTGAAGTTCAAGTTTTATTAAAAGAATTAAATTTAAAGGAGGATATA
AATTGGGATTGGTTTATTCTATTAATCATTAAAACAGGACTTCGATTTTC
AGAAGCATTAGCTCTAACACCTTCAGACTTTGATTTTTCCACACAAAAAA
TTAGTATTAATAAAACCTGGGATTACAAAATGGTTACTGGTTCTTTTCAG
CCAACAAAAAACGAATCTTCCAATAGGAAGATTCAAATTGATTGGCAATT
AGCAATGCAATTTTCCCAGTTAATTAAAATGAAAGATTCTGATAAACCTA
TCTTTGTTAAAAGTAGAGTATTCAATTCTACTATTAATAATCGTTTAAAA
GTGTTGTGTCAGAATGCAAATATACCAACTATTACAATTCATAGTTTACG
ACATACCCATGCATCTTTGCTATTATTTGCTGGAGTTTCAATTGCTAGTG
TTGCGAATAGGTTAGGTCATTCAAGTATGACTACTACCCAGGAAACATAT
CTTCATATTATTCAGGAATTGGAAAATCAAGATAATGATAAAATTATACG
CCATTTATCAATGTTAATGTGA
ORF3792 (SEQ ID NO:128)
ATGGAAACAATAAATCATTTTTTTGAATGGGCTAAAAATAATAATTGGCA
AGTAGATTTGTCTGCTGTAGAGAAAAACTTGCCAGAGCAGATTTTAAAGA
GGTATGGAAAGTTGCCAGATGCTTATAAGGCTTTTTATAGACAACTTAAC
CTCTGTAGCAATGCGGGTGACACTTGTTGGTTTTTATCGGAAGAAGATTT
TTTAGAGAATGAGGTTGACGCCTTTTCGTGGAATTCTTTTGAACAAATGA
GTTTAGAAGCAGCAGAAGGCGATACAAACTTAGAAAATGAAGTAAAACTA
TTTTGGAATGCGCATTTACCAATTATGATGAGTGTTGGCGGTTGTTATGA
ATATTATGCTATCACTTTAAATGACGGAAATATTGTTCATGGTTCGGAAC
CAGAGTTTGAAGAAAGTTCGATTGTAGCAGAGTCGTTCGCGGATTTTTTA
CTGAAAATCGTGGCTGGAGAAATAGTGATAAGTTAA
ORF3793 (SEQ ID NO:129)
CATAACGTAAATACTCGGAGGTGCAAAATGAAGGTTATCCATTTTTATAG
TGAAAATGCGGACTATGGCTGTTTCTCTAATTTCTCTTTACATCCAATTG
TCATAGACGGCGTGACTTACCCAACCACAGAACACTATTTCCAAGCACAA
AAATTTATAGATAAAAAAATTATTAAAAAAGTAATTAATACGAAAAAACC
AATAGATGCAGCAAAATTGGGTAGAAATAGAGACTTCCCACTGCGAAAAG
GCTGGGAAAGTATGAAAGACGAAGTGATGTTAAAAGCAATCAGAGCAAAA
GTAGCGCAACATTCGGAAGTGAAAGAAATGCTTTTATCCACAGAGAATGC
TATTTTAGTGGAACATACGGAAAATGATCATTATTGGGGCGACGGCGGAG
ACGGCTCTGGAAAAAATCGTCTAGGCAAGATTTTAATGAAAGTCAGAGAC
GAATGA
ORF3840 (SEQ ID NO:130)
GAAAACATAAACTTGATGGTGGTGAAACAAATGGACGACTGGTCAACTTT
GATAGGCGAGCTAGAACATAATACCCCAGAAAAAGTTTGGATTGTTAGAG
AAGAACTAAACATATCGGAAGTATTTGAAATACAAAAATTAGAGGCGCAT
TTTATTTTAGTTTTAGAAGGTGTCTTGCGTATGGAAAATGAACATCAACA
AATTCTGCATTATTTTAGGGAGAATAACGTCATATATCAATCTCCATATG
AATTAAGAGTACAAAATAAACTAAGACTAGTTGCTGAAACACCTGCTCAC
ATTGTTTTACTGCACCGTGAGTTTTTCCTTAATTATGCAACAAACCAACC
TGCTTATTCTGAAAAATTAGTGCGAGCAATTATGGATAATGCCGCGAGTT
TTATGTTTGAGTTAATGAAGAATGACCTTAAATCAGAGGACCGATTGGCT
TATTCATTACAGCAGCTATGCCAAAGTTTAGAATTAGAAGTAAAAAATGA
GTTTTATCTACTGCCTCATTATATTAATAAGAATAAATTGGCGCTTTATA
GTGATATTTCGAGGAAAAGTTTATACAAATACCTTCAAAATTTAGAAAGA
AAAGGACAAATCAAAATGAATGGTAATCAAATACTTGTCCGCATTTCGCG
ATTCACAAATAGCGAGAATGTAGATTGGTTGTAG
ORF3913 (SEQ ID NO:131)
GAATCCATTTGGAGGTGTGAGTATATGCTAAAAAAAACGAAAAAAAATCT
CAATGAAATTAAAACTATTATCCAGCAAAAACCTTTTGATTATGAGAAGG
CAGAAAATTTGATAAGTGAGTTAAATCTTGAAGAGGCCCTACTAGAATTG
GAGAACATCTTACTAGCTAGCATAAATAATAGCGATGAGAACGCTCGAAT
ATTTGCCTATGAGTATTTATATTATTTTGATAGCGAAGCAGTTTTTCAAG
CAGCCCTCATAGGGACAACGGACGACGATGATTTAGTTCAAATGTGCTCC
ATAGAGATTTTAGGAAATTTGGTTAAAGTAGAAAGTCTCCCGTATTTAAA
GAAGGCGTTGGGCGATAACAACCCTGATGTACGTTGCTTTGCTGCTGAAT
CAATCGGTTTTGTAGGGACTGACGAAGCGAAAGCGATACTGCAAGAACAG
TTAAATAGAGAAACGGATTCTTTTGCAAAAGTAGGTATTTATTATGCGCT
CTATCTTCTTGGTCGAGAAGAAATGTTGCCAAAGCTCCTTTCTCTACTAG
ACGATAATTATCATTTGACGGTGATTAGAAGCTTGGATGTTCTGAGAGAC
GTTGTTAATCAAACAAATAAAGAAAATATATTATTAAATATCGAAAAACT
TTTAAAGAGAGATATCCCAATTTCAGTAAAGGAAAAAGCGGAAGTGGTTT
TACAAGAAATTAAAGGAAGCTAA
ORF3914 (SEQ ID NO:132)
ACGAAGTCCAAAGCATACACAGAAAAGAGGGCTGTTATGATAAACCTAGA
ATGGAAAGAACTAGACCAACTGGAGATAGAAGAAAAGGTTCAAGAGGTAT
TAGATTATAGCTATAATACCTGGATGTCTGATAAGAAGAATATTCGTTAT
TTTGTACGGGCGTTTTATATTAGATGGGATATGTTGGTTGATATGTATGA
AGTGGAAGATGATGAGACGGAAGGCGATAAGTTAAAATATATGTATGATT
TCGGCATTAGCGAGCTGGGAAATATTACAGAGGTTGACTGGATAATGGGT
TATTGCATGCTAATTAATCCAATCTATTTTGAAGAAAATGATAATTATTT
GGAACTTGAGGAGAAAGGGCAGGAAATGCTTCGTAATGTAGCAATAAATA
ACCCGGATGACGTCTTTTTGACTTCTTTTGGTATACCTGAAAAAGATTAT
TTGAAATGGAAAATAGCGAACAGAGAGCAACTTATTCAGTACGGGGAAGA
TAATTTTAGCTATGACTCAGAGTTTTCGAGATATTTTAAACATATTATAA
ATTGCAGGGCGAATGAAGAGGTGGAAAAAGAAAGCTTTCTGAAGAAGATT
GTGCGCAGGTGGAAGCAACGATGA
ORF3921 (SEQ ID NO:133)
AAGAGGCATCTTGTGTTGCCTCTTTTTTATGTAATTTCTCACTATTCTAA
CTTACCATTTCGAGCTTGGGAACAGCGTAAGTTAGGGGACTTAGTTGTAG
ATTATGTTGAAAAGACAAGTGTACAAAATCAATTTCCCATGCTAACTTCA
TCTCAACAAAAAGGGATAGTTTTGCAAGAAGACTATTTTGCAAATCGACA
AGTAACCACAGAAAATAATATTGGTTACTTTGTACTACCTAGAGGATATT
TCACATTCCGTAGTCGAAGCGATAATGATGTGTTTGTGTTTAACCGTAAT
GACATTATTGATAGAGGGATTATAAGCTATTTTTATCCTGTATTTACTTT
AAAATCTGCTGATTCCGACTTCTTCTTAAGGCGTATTAACAATGGAATCC
AACGACAATTATCTATTCAAGCAGAGGGAACAGGTCAACATGTCCTGTCT
CTAAAAAAATTTAAAAATATAGTAGCAATGTTTCCTAGTGAGGGAGAACA
AAAAAAAATCGGTTCTTTCTTCAAACAACTCGACGACACTATCGCTCTTC
ATCAGCGTAAGTTAGATACTTTGAAACAGATGAAGAAAGGACTCTTACAG
CAAATGTTCCCTAAAAGCGAAGAGGATGTCCCAAAGATTCGTTTTGCTGA
TTTTGACGAAGAATGGTACCAGCGTAAGTTGGGAGAAGAATTTGAAAAAA
TAAATGAGCGTAATGACGGTTCTTTCGGCAAAACTCATTGGATATCAGTA
GCAAAAATGTACTTTGTAGAACCTAATAAGGTCTTATCAAATAACATAGA
TACAAGGACATATGTAATGCGAAAAGGTGATATTGCATTTGAAGGACATT
CAAATACCGATTTTAAATTTGGGAGATTTGTAGCGAACGACATTGGCCCA
GGGATAGTATCGGAATTATTTCCAGTATATCGTCATAAAACTAACTATGA
TAATAATTATTGGAAAAATGCTATTCAATTAGAGCATATAATGGCACCTA
TTTATTCAAAGTCTATAACTTCATCCGGTAATTCATCAAACAAACTTGAT
TCAAAGCATTTCTTAAATCAAAAAATTTATATTGCTGACTTTGAAGAACA
AGAAAAAATTGGTTCAATTTTTAAACAATTGGATAATACCATTATTCTCT
ATCAAAATAAATTAAATAAATTTGATATTCTCAAAAAAGCTTATTTGCAA
ACTATGTTCATTTAA
ORF3970 (SEQ ID NO:134)
ATAATAGCTCTTTATTCAACACGTGAAGGTTTCGAGAAAAGATATGGCTT
TGTTGATGTTGATAAAGATAATAGCTATAAACGTTTGAAGAAGAAAAGTT
TCTATTGGTATAAAAAAGTAATTGAAACTAATGGAAATGATTTAAGTTAT
TAA
ORF3981 (SEQ ID NO:135)
CTGGAGGAAAATCAAATGAAAAAAGTATCATCTTTATTAACCCAAGATCA
GTTTTGGGGAATTATTGATAACTCTGACAAAGGCAGCAAATTAGAAGAAC
TACTAGAAAAGTTGAGTGAGGATGAACTTTTTGGTTATGATTATTGGTGG
AACTACTTTCATAAAAAATCTTATAACCAATCGCTCTGGGCTGTTGCTTA
CGTAGTATTAGGTGGCTGTAGTGATGATGGATTTGACTATTTCCGTTATT
GGTTAATAACAAGAGGTAAGGCTGTATTCACATCTGCTGTGGAAAATGCC
GATACTCTTTGCGGTGAATTTGATTTATTAACAGAGGATGAATATCCTGA
AAATGAAGAGGTAGCTTACTTAGTGATGGATATTTTCGAGAACAAACTAG
GGAAAGACTTTGACGATGCTGAAAATGAAGCAGAGAGTAGAATTGAGTTT
GAAGAAGTATCTATGCCTCCAATAGATTTTGAGTGGGATGAAGACGACGA
AGATTCTATCAAAAAAGTATGCCCAAACACTTTTGCAAAATGGTGGAATA
ACGACAAATTCTAA
ORF4017 (SEQ ID NO:136)
ACAGGGGGGAGTATCCTGATGAAGTTAGTACAAAAAGTGTATTATTTGTT
GTTTAGATTAGTGGGATTTTTACCGCGAAAAAAAGATTTAGTGATATTTG
AAAGCTTTTCAGGTAAGCAATATAGCTGTAATCCACGGGCTATTTATGAA
TATATGGAGGAGCATAATCCGGAATACGAATTATTATGGAGTGTTAATCC
AAAATTCGTGGCTGTTTTTGAAGCTTACGGAGTTCCTTATGTTAAACGTT
TTTCTATTAGCTGGTTATTTAAAATGGGGCTTGCTAAGTATTGGATTTCT
AATAGTAGACTTCCTTTAGAACTACCAAAACCGAAAAAAACAATCTATGT
GCAAACTTGGCATGGAACACCCTTAAAAAAATTAGGCGTGGATATTGATG
AAGTGCACATTCCAGGGCAAACAACGGAACAGTACAAAGCTGATTTTGTA
AAAGAAGCACAGAAATGGGACTACTTAATTTCTCCTAATGCTTATTCTAG
TGCTATTTTTAGACGGGCATTTGGTTTTACTGGAGAGATGATTGAGTCGG
GATATCCGCGGAATGATATTTTATTTAGTACCGATAAAGAGCTGAAAATA
GCTAATATTAAAAAAGAGCTAAATATCCCAGAAGAGAAAAAAGTTGTTTT
ATATGCGCCAACATGGCGTGATAATGATTTTTATGAAGCTGGAAGATATA
AATTTGATTTAAAAATCGATATCGCAAAAATGCAAGAAAAATTGGGGAAT
GAGATTGTTTTACTTGTTCGCATGCATTATTTAGTCGCGGAACATTTTGA
TTTCACGCAATATGGTGATTTTGTCCGCGATGCTTCAAACCATGAAGATA
TACGCGATTTATACTTAGTGAGTGATTTGTTGATTACTGATTATTCCTCT
GTATTTTTTGATTATGCGAATTTACAGCGTCCTATGCTTTTTTATACGTA
TGATTTGGCGGAATATCGTGATACGTTACGTGGCTTTTACTTTGATTTTG
AAAAAAATGCTCCTGGTCCGCTTGTGGAAACGAATGAAGAATTAATGAGT
GAACTTGAAAAAATGCTTGAAAATCCACCTAAAATAGAAGACAGCTTTTT
GGAGCAGTTTTGTACTTGGGAAGATGGTCATGCAGCAGAGAAAACAGTGA
AAATCGTTTTTGCTGAAAAATAG
ORF4018 (SEQ ID NO:137)
GTGGTGGGAATTTTGAAAGAAGTAGCAATTTATATTTATATGCTTGCTGT
TAAGATAACTGGCTGTTTGGCGAGGATTTTTCCAGTTAAACAAAAAGTGG
TACTGTTAGTTAGCTTTCCGGAAAATTCCACTGCAATTATAAAGCAAATG
AACGAAATGAAAGTTACGCCAAAAACGGTTGTTTTCTATGATCCAAGAGT
GGATGTGACCGGTTTTAATTTTGATTTTATCCAATTAAAGCCAAAAAAAA
TCAAGCACTTTATCTCGTTAATGTTCCATCTGAATACGGCAAAAGTTGTC
ATTACGGATAATTATTTCGTGGAATTAGCTGGATTAAAAGAACGTAAAAA
TGTGACGTGCATTCAAATTTGGCATGCGAATGGGGCATTGAAAAAATTCG
GCTGGGAAGATAAAGCAGCGCAAAAAAGAAGCGCGCGCGATAAAAAAAGA
TTTCAAGAAGTGTACAGATGTTTTTCAAAAGTGCTCGTCGGATCGGATGA
AATGGCAGCAATTTTCCAAAGGTCGTTTTTACTAGAAGATTCGCATATGT
TGAAGCTAGGAATTCCGAGAACGGACAATTTCTTCAACCAACAACAATTG
AAAGAAAATGCCGAAAGGACAAATACCAAATTACATCTTTCCAATAAAAA
GAAATTATTATATGCGCCGACGTTCCGCGATGAGGAACTTCATAGTACTA
CGCTTCATTTAGATATCGCGAAGATGAAACAAGCACTCGGAAATGAGTAC
CAATTAATTTTAAAATTACATCCATCTATTAGTAATGATTTAGATGAAGT
AGTGGATGATTTTGTTGTCTATGCGGATAAAGAAACGCCAATCGAGACGA
TACTACCAGCCGTAGATATATTGATTACTGATTATTCATCTATTCCATTT
GAATTTGCTTTATTAGAAAAACCAATGATCTTTTTCACGTATGATTTAGA
GGAATATGATAAGGCTAGAGGTTTGTCGGATGGCTTTTTAGCAACGATTC
CGGGGCCATTCGTTCATACAACAGAGGAACTAATACAACTAATTGAGCAA
GAAGCATTCGATTTAGAAATGGTTCGTGCTTTTGCGGCGAAATGGAATAA
ATATTCAGACGGGCATTCTAGTGAGCGCTTCGTTTCCTTTTTGAAAGAAC
AGCTAGAAAAGTAG
ORF4100 (SEQ ID NO:138)
ACAGGAGGTACTGATATAATGAGTTATTTATGGACAAGTATTAAAATGCA
ATTTAGAATTCCAGTATCTGTGTTTTTCTCATTATTATTTCCACTAATCA
TGATGTTCGCTATGGTTACTTCTTATGGAAACTTTGATATTGGTGAAGGT
TATCATTTTGTAGATAAATATTTTTTGATTAGTACAGGGATGGGCATGTT
ACCAATAGCATTAATCAGTTTTCCAATCTGGCTTGGTGAAAGTGTTCAAA
ATAAAAGCTATAAAAGATTAGAATATTTTGGATTGAGTGCACAGAAAATA
ATTGTTTCAGATGTATGTTCTTATATTTTGCTAACAGCCTTAAGTATTTT
TGTCAATATACTGTTTGGCTACTTGGTATATGGACTACATATCCCAGACT
GGCAATATTTTATTGCTTATGTTTTGCAATGTCTGTATTGTAATCTAGTG
CTTTTGATTTTTGGTGCCTTGCTTGCTTTAATTATTAAAAATCCCCGGAT
ATTAATGCCAGTAGGAATGTGTTTATTATTTATGTTTTATATTTTTACAG
GAACATTTTCATCTTTTTCGGAACTTCCTAAATCATTTCAAGCAGTTGGT
AATTTTTTACCTATGAAATATATAATGAATGATTTATTTAATGTTTGGAC
ACAAAATAAATTGTTCATCTCTAAGTTTTTAGCTTTAAACACGATATATG
GAATCATCTTATCCTGCGCACTAATTGCTTTTTTGTTGAGAAGCAGAAAA
ATAAAAATTAAATCATAA
ORF4101 (SEQ ID NO:139)
AATGAAAAAGGGATACATTTGTGTCCCTTTTCAATAAGTTATGAGGTGCA
TGTGCATATGAATAATATTTTGGAAACGAAAAATTTGAAAGTTACAATAA
ATAATAAAGTAATTCTATATTTAGATAAAGAAGTTTGTATTTCAGAAAAA
GACAAAGTTGCCATTCTAGGAGACAATGGAGCTGGGAAAACCACGTTAGT
AAATAGTATTCTTGGTGAAAAGAACTCTTCTGGAGAAATTACCAAAAAGT
TTAAAAAAAATGACTGTGGTGTAGTGTTTCAAGAAAATGCATATAATGAT
TTGATGAAAGTTTATGAATTGATTACTTTAGTTCTGCCACATCTAAAGAA
AAAAGAGAGAGCGCAGTTTTTACACAAATATGAACTTGAAAGTTTGAGAA
AAAAGTACATTAAAGACTTATCTGGTGGAGAAAAACAACGACTAACACTA
TGTTTAGTACTAGAAAGTCATAAGAAATTATATATCTTTGATGAGTTAAC
TTCAGGATTAGATTATAAGAAGAGACTAGGTTTACTTGCTTTGATGAAAG
AAAAAACTAAGGACGCGACGGTGATAAATATAACGCATTACTTTGAGGAA
GTTGAAAACTGGGCAACGAAAGTTTTGATTCTTCAAAAAGGAATTTTACT
TTTTTGGGGAACAATATCAGAATTTTTCTCTAATTTTCCTCATTATTCTG
TTATTAAAGTAGATCAAATCGAACTAACAAAGATAGATGAAACAGATATG
ACTTTTATGCAAAGCACAGATACAGGTGACGGGATAGCAGTTATTTGTTC
GGATTTGCAAATTCAGGAAGAGACAAAGAAAATTTTGGATAAAAAAAACG
TCACATACAACACAATAAAACAAAATATATATACAACTTATTTAGTTGCA
TATTTGCGCGGAACATCAAGCAGTGAACAGGAGGTACTGATATAA
ORF4132 (SEQ ID NO:140)
TTACATCTAAGGAGACTAATCATGAAAAGAAAGATAAGTTCTATCATTGT
AGTCGGGATAATGTTCTTTCAATCATTAACTACGTATCCATTTATCACCG
AAGCCAAAGAAAATGAACAAAAAGAAGAAATAAATAAACCCTCAAAAATA
ACTAAGGGATTAACTAATTCCCTAAAATACACTAAGACAATTCTTGAAAC
AGGGGACACCTATGATAGTGTATTTCCTGACAGTGCTTTAGCTAAAGTAG
TTGCTAAAGAAGCAACGGGGTCAGAGAATACAACTCAGCTAGTAACACAA
GCAGACCTGAATAAAATCAAATCTCTCAATGGTTATAATAAAGGAATCAG
CGTATTAACAGGAATCGATTTGCTAGTCAATGTGACAAGTATAAGCTTGA
ATAATAATCAAGTAACAGACATTTCTCCAATAGATCAATTACCTAATTTA
GTAAGCCTATCGGTAAAAAACAATCAAATTAGCAGTCTTATCTTAAATGC
GCAAAATCAACTCCCGAAACTAACAACCATTGATATTGAAAATAATCCTG
ATCTTAATACTATCGATATCCAAGATCAACCCCAATTGGTAGATGTAAAG
ACATCCGGTTATACAGGATTACGCAAGTTAACAACGGTAATTGCCAAAAA
CAATCCAGAATTAGTCAATTTAGGCCAATACACTATCCGAAATGTCTACT
TTAGCCAAGTAGCAAGTTTAACGAAAGTTGAATTAGTCAATCTCCCTAAG
GTAAGAAAAGTAAACCTTGAGAGGAACAGTATTAATGAGCTAAAAGTTAC
TGATTTGGCCATTGAAGATCTGCCATTAGGAGAAAATGAATTAACAGATA
CTGTATTTGATAACATCCAAAATCTGCCTAATTTAAAAACGTTAGACCTG
TCAAAAAACCAACTAGAAGAAGTTGTATTAGACAAAACGGATGTAGAAAA
TCTACCCAATTTGATGACACTAAATATACAGCAAAATTTGGCTATAAAAT
TGATAAATGTTCAAGACCAACCCCAATTGGTAGATGTAAAAACTTCTGAT
TATAAAGAATTATCTGCGTTAACAACAGTAATTGCCAAAAATAATCCAGA
ATTAGTTAATTTAGGTTATCCCATTATGCAAAATGTTTACTTTAGCCAGG
TGGCAAGTTTAACGAAAGTTGAATTAGTCAATCTCCCTAAGGTAAGAAAA
GTAAACCTTGAGAGGAACAGTATTAATGAGCTAAAAGTTACTGATTTGGC
CATTGAAGATCTGCCATTAGGAGAAAATGAATTAACAGATACTGTATTCG
ATAACATCCAAAATCTGCCTAATTTAAAAACGTTAGATCTGTCAAAAAAC
CAACTAGAAGAAGTTGTATTAGACAAAACGGATGTAGAAAATCTACCCAA
TTTGATGACACTAAATATACAACAAAATTTGGCTATAAAATTGATAAATG
TTCAAGACCAACCCCAATTGGTAGATGTAAAAACTTCTGATTATAAAGAA
TTATCTGCGTTAACAACAGTAATTGCCAAAAATAATCCAGAATTAGTTAA
TTTAGGTTATCCCATTATGCAAAATGTTTACTTTAGCCAGGTGGCAAGTT
TAACGAAAGTTGAACTTGCTAACCTTCCAAAAGTAAGAGCAGTTCGATTG
GAACGTAATAGCATCAATCAGATTGAACTAAATAATTTAGTTTCAGTAAA
AGACGTGAATCTAAATACTAACAAAATAACAAATGATAGTATAGAGAAAT
TTAAAGGCATGCCTATACTAGCAACTTTAAATTTAAACAAAAACCAAATT
ACCAATATAAATATGCTTGATGATTTCCCAGAAATGACTACTTTAAATAT
TGATTTAAACTCCGTCAGTGTTTTACCAAGTAACCTTAAAACAAAGATGC
CAAAACTATCGAGAATTAGTGCATTAAATCAAACAGTTACTTTAGATAAA
GCAATTGTAGTGGATGATTCCGATTTAATTATTAATAATGAGATTAGTAA
TTTTGGAAAGTTAACCGATCCAAGCCCAATATCAAATTTTGGAACTTACG
CAAATGAAAAAATAACTTGGTCGTCTGAAAGAATTAAAAATTTAACAGAG
GTTAGTTTTAAGTTTTCTGAGCTAATTAATGTGACTGGTATAGATGGAAC
TTTTTCAGGAAAAGTAACACAACCATTTAAAAAATCAACTACACCAGTAA
TTAATGCAGATTCCGAAATTCATTATCCGCAAGGGACAAAAAAAACAGAA
GCAGAGTTTTTAAAGGATATTCAAGCGCAAACAACGGATGATTTATCCAT
TAAAAGCGATTTTGAAATAATGGTGAATCTCAAGAAAGTAGGAAAATACA
CAGTAATATTAAACGTAGAGAATATGGATGGAATTAAAGCTAATCCAAAA
GAAGTGACTGTTTATATTGATGCTGTACAAGGTGCAAATATAACTGTGAA
ATATGAAGACAAATCAGGAAATAAACTTGCAGAAAATAGCATATTAACTG
GAAATGTCGGTGAAGAATATAGTTCAAGTGAAAAAGAAATTTTAGGTTAT
ACACTAACTGAAATTCCAACAAATGCACAAGGAGAATTTAGTTTAGAAGA
ACAGACTGTGACATATATCTATTCGAAAAATCCCGTTCCAGCTAAAGACA
TCACGGTACAATATACCGATGAAGATGGAATAGAACTAGCACCAACCGAA
ACATTATCAGGTAATGTTGATGAAAATTATGTTACAACAGCGAAGACTTT
TACAGGATACGAATTAATTGAAACTCCGAGCAATGCAGAAGGAAAGTTTA
GCGAAAATGCACAAACAGTGACCTATGTGTATCGCGCGATTAAAGCAGAT
CCAATTTTAGCAAAAGAAGTTACGGTGAACTACCAGGACGAATTAGGAGC
TAAAATTAGTGAAACGGAAGTATTAACTGGTGAAATTGGTGAAACATACA
CAACAGTGGCTAAAACAATAGATGGCTATACCTTAATTAAGAGCCCTATT
AACGCTAGCGGGATTTTTAACGAAAATCCTCAAACGGTTACCTATGTGTA
TCAATTACAAAACAACCCAATAACAGCAAATATCACTGTTAAACACTTGG
ATGAAAACAACAATGAGCTAGCACCGAGTGAGGTACTAAGTGGAATTGTC
GATGAAGCATATACAACTAATCCAAAAGAAATAAAAGACTATTCGCTAGT
AAAAGTACCAACTAATGCTAGCGGAAAATTCACGACAGAAGCACAAACGG
TGATTTATCACTATAAAAAAAACAGTATACAGACATCATCTTATATTACT
GTAAAATATGTAGATGAAACAGGGAAAGAGCTGGCCATAAGCGAGGTATT
AAACGGAAACATTAATGATTCCTACGCAACTACAGCTAAGGAAATTAAAG
GCTACACGCTAGTGGAAAAGCCCGCTAATGCAACTGGTAAATTTACAGAT
CAGGATCAAACTATAAAATATGTATATCGTGCTAATACAGATGAAGTAAA
TCTAGACCCAGATGTACCAGCGAGAAATCCCAATGGAGATATTCAACCAA
TGAATGGTGCCCAACCACCAATTGAGGTTCCAAAATCCCTGCCTAAAACA
GGTAGCCAGCCGGCAAATTTGATCTTTGGATTAGGAGTTTTACTAGTGTT
TCTTAGTACCCAATGGCTACATCGAGATAAAAGAAAAAAAGATAAATTTC
ATAACTGCTGA
ORF4133 (SEQ ID NO:141)
CACTTGATGGAAAATTACGTGTCAATAGTAAAAATCGAAAACAATCTTTC
CGTGTGCTTTTACAACAGCTCGGAGAAAGTAGTAGCAATTGCTAAGAAAA
TGAATGAGATTAACGAAGAAGCTTATATGCATGGTTACAATTGGGAAGCA
TTTTTCAACTACTATTTACCTAAATATGCTCCAGATGTCTTAGAAGGAAT
GGGCTCTGATCCGGAAGCGGGAATGTATGTGGCGTATTACACGCTATCAC
CTGAAACTGAGGCACGAGCAGAAAAACTTGTTCAAGTAATTACGAATCTC
ATCGAAAATGAAGAACTACTTTATCAAATAATTGAAAATGAAGGCAATAA
TATTAGTTGGGATAATTAA
ORF4135 (SEQ ID NO:142)
GAGGCAATTATGACTTTTTTAAACACCTTAAAATTAAATTTGGAAAATGA
AAAAAAGAGAATGTTATCCGATGCTTTTATGAAAAAACAAGAAGGAATCA
TTGTAAACTATATAGTGACTTGCAGTAAGGATTCTGCTATTGGCATTAGT
AAAAAGGCAATTGATATATTATTGATAATCAATGAAAATACATTTCCTGA
ATGGCCAAATGTAGATAGATGGCTTTCTATTTTGCCAAAATATTTTACGG
ATTCTTTTTCAAAATCAAAAATATTGCATAGTGAAGATTGGCTATTTGAA
GAGTGGTTATACTGGTTTGAACCTGAAAATAGATTTTGGTTTTTAGGAGA
ATTAGATCCTGTTGATAATGAGCATTTGAAAATAAGCATAGTTGTACAAG
AACACCCTTTTCCAGTAGAATCATTAGAAGTTCTACTTATGAAGCTAGGA
ACAAGCGAATTACATGAAATTGGTATGGAATGA
ORF4188 (SEQ ID NO:143)
AATTTTAGGGAGATAGATATGAATAAAGTGACGAAAATTGGAGATGTAGC
AGAGAAAACGGGCTATTCTATAACTACTATTTCTCGAGCAATTAATGGTA
ATCCAAATGTTTCAGATAAAACAAAAAAGAAAATTTTTGCTGCGATGAAA
GAACTGAATTATTATCCTAATAATATTGCTCAACAATTTCGAGGGCAGGG
GACTAAGATGATTGGTGTTGTGATTTCCTTTATAACCAATCCTTTTTTTG
CGTACCTTGTTGATGCGATTGAACGCTATCTATCTCACAGAGGGTATCAG
GTTGTCATGTTGCAAACTTTAGAAAATCCTGCAAAAGAATTACAATTTAT
CGAGATGTTACAAAAGAAACAGCTAGATGGATTGATTATGGCTAACTTAG
AAAATGATACAGAAGAAATTAAATCACTCGTTGAAAGTGGCAAAATTGTT
CTTTGTAATCGTTATCTGGGAAATGAAAATTTGACGATTATTAACATTGA
TGAAACGAAAGCGGCTTATCAAGCAACGAATTATTTAATAAAGTGCGGTT
ATAAACGCCTTGCCTACTGTACTGGTGGTATTAAAAATAAAAATGATTAC
CGTTTTAAAGGATTCATGCAAGCTGTAACAGAAAATGGACTTTCTTTTGA
TGAATCATTGTATTTTGAGAAGTTATTAACCATTAAAGATGGCGAAGAGC
TTCTAGTTAATATTTTAGAAGAGAAATCTACTCTGCCGGACGCAATTTTT
TCAAATGGTGATACGGTTGCTGCTGGGATATTATATGCTGCAAAAAAATA
CGGGATAGCAGTGCCAGAGGAGCTAGGAATCATAGGTTTTGATAATCAAC
CTATAGCGGAAGTTCTGAATCCGGCGTTGACAACTATTGAACAACCAATT
AAAGAACTTGGTGAATATTCGGCGCAAGTTCTATTGGCTAATTTACAAGG
AACGAGTGTGCCGGTAGCGCCAGACCTTGAAACAAAACTAATAATAAGAG
AGACTACCAAGTAA
ORF4327 (SEQ ID NO:144)
GAGAATGGCAGAAAGAAGAATTTGGCGTTTCTCCAGAAGAGGAGGATGAG
ATACTTGAATTATGATGAACGTGTACGAGTTTTAATTGAGCTGAAAGTTG
ATTTGTCCGGAAAATTAGAAATGATGGAAAATGAAGAAGAGTTATTATGT
AGACAAAAACATGATTTTGTAAGTACCTGGTCAAATGCCAAAACAGAGGA
TGCTTACAGAAAATTAAATGAAGCAGTTAGAAAGAAAATCAAAGAAACAA
CGGAATATGCAAGAGAAATTGATGAAAAAATTACTGCTCGTATCAAACGA
ATTGAAGCAGCATATAAAGCAGAATATCAGAGTAATAGGAGCTATACATG
GCGTATCGCAGAAATCGATCCAATTAAATTTAAACAAAAGTATAATGAAC
GATTAAATCAGTTAAGCTACTTATCATGTGATGGCAGCGTAAAAACACGT
TTAATAAAAGAGTTTCGGCAAAATAATTTCCTGAAATAG
ORF4369 (SEQ ID NO:145)
ATGGAGAGCGAACTAGAGGGAGGAAACAACATGAAAAAAACACGTGTCAC
ATGGGCAATAGTTATTGCTATAGCAGGCTTGTTAGTTTCTGTGATCAGTT
TATCAGTAAACATCACCCAGAATAGATATAATGTTCGAGGAGTGGATTCT
AGTTCCCAGTGGAAAACTGGATTTCAAAAAAATCAAACTGTTTGTGCAAT
AGAAACAAAGAAGCATTGGACAAAGGGCAGTGTGAAGTATGGTGTGAACC
AAACTTCTGGAACTGTTAAGACAAAGTATAGTATATACAGCAAAAAAAAA
TAA
ORF4457 (SEQ ID NO:146)
AGTAGAGATGAGATTAACAAAATGAGAGCAGACTTATCAATTATGTGGGT
TGAAGATACAACTTCATGGTATACAGAACAAAAAGATCTTCTAAATATGG
ACATAGAAAGCTTGGGTATAGATATTGAATTTACTCGTATTTCTACAATT
GATGATTTTTATAAATTGGTTAGGAATGATAATACAGGTTTTAGTAAATA
TGATATTTTTTTTGTTGATTATGCATTATCCTCTGATACTGAACAAAAAG
GAAGTAACATAATTGTTAAGTTAAAAGATATGGGCTTAACAACAGATATT
CTTTTCTATTCTTCAAAAAATATAAGAGAAATAAGGGAAATTGTGAAAAA
AGATATTGAACAGTTTGAGGGAATATATCTAGCTGATAGGGATAAAGATT
TCAGGGATAAAAGTTTTCAATTAGTTGAAAAAAATATTAAATCATTATCA
TCTATAAAAAATATTAGAGGTCTATTAATGGATCAAACTTCTGAAAACGA
TTTTATAGTTAAATCTTATATTTCAAAAGAAATAAATGAACTTTTACCTG
AACAAAAAGAAAAAATATATAAATGGTTTGAGAAGGAAGTAGATAAAAAT
ATAGAAGAAGTAACGAAAACAATTAAAGGTTATGTAGAGAGAAAGCCCAG
TACTATAGAAAAATATTTAAAATTACCAAGTTATATATTACCAGTTAAAC
TACAATATAATTTATTAGAAGAAATTATAAATATTAATGGAGTAGAGACT
TTTGTTGATTCAACTATTGATTATTATTTGAAAAAAGTTGTGAGTAATAG
AAATAAATTAGCTCATAGAAAATTAGTTATGTGTAGGCAGCAACAACATT
TATTGCAGTATAATAATATTAATACGTTTCTAAATGATGATTCTAATGTT
TGTGATAATCATGAAGGAGATGTAGTTTCAATAGATGATTGGATTCAACT
CAGAGTTGAAGTTAAAGAATATGGTAAGTTATTTGATGATTTGTGGGAGA
AAATTAAGTCGTGA
ORF4507 (SEQ ID NO:147)
AAAGGAAACAGCAAAATGATCGACTATGAGAAAAAAGGCTTTTTTAACAT
CCACACATTGGTAAATAAAGATAATGCTAATATTTCTAATAGTGATAATA
AACATATTTATTCCCAGCTGATGTCTGGTAATGGCAATTCACCAATGCTA
GGTTATTTATTAAATATGAATAAACAAAACTTGAATGACTTCAAAAGTAT
CATGTTTTATAATGAATCTAATCTAGCTTCTTTAATTAACGAGGCAAGAG
AAATGGAAGAATTAATAGATAGCTCAACTCTTTTTTTAAGTAAAACAAAT
AAAAAAATTAAAACCCATTTTTCTAAAGTGCTTGAGCAAAGACATAGTAC
AAGAAATTTTGTATACGAAACCATGGATTTATCAACATTCTCAAATATCA
TACAGTTTTCTTTTGGCCTTAGTACAAGAAAGTTGGTTTATAACGACTTA
CAATCAACCACCAGGCATTATTCTTCTGGGGGCGGTTTATATCCAATTGA
TGTTTTTTTGTATATAAATAATATTTCTGGAATTGCCAAGGGTATATATA
AATATCAGCCATACACGCATAGCTTGCATCCTCTAGATGTAGATAAAATT
GATGTAGAGTCGTTTTTCGTAGGTGATAATATTGACACTTCTAATATGAA
TTTTTGTGTTTTCTTTGGATACTCTATTAATAAAAACTATGTGAAATATG
GAGAACTATCTTTACTGAATACGTTTGTGGAACTCGGAGGAATATCGCAC
AACTTTGATTTAGTCTGTCATTCGGTTCACTATACAAGTTGTCCTATTGC
AGGTTTTAACAAATCATATTTGGAAAAGCTTTTATATTTAGATGGAATAA
ATGATCACATTATATTTTCTAATATTTGTGGAAAGGAATGA
ORF4563 (SEQ ID NO:148)
TCTATGAAAAAAAAATTTAGTAATCCCACGTTTAGAATTATTGCATCAAT
TGTTCTAGGGATTTTAATAGGTGTTCTTATTTGCTTTGTGGCTATTGGTT
TAGGTTATATACACATGAATGATGGCACATTAAAAGAGTATAGTGTGAAA
ATTTTCGGACTAACTATTTTTGATATTAAAAGAGTTGGTAGTGAGATGGT
GGGAACACCTAATAACACTAGCATGATGTTTATAGGGGTTATTATTTCTA
TGATACTAGCTATTGTTGTAGAAATTATTGTTTCGTTAAAAAACAGACAT
AGAAAGGAAACAGCAAAATGA
ORF4578 (SEQ ID NO:149)
ATATATACAATTTTTGATAATTATGCTATATTTGGAATGCACATAAATAA
TTTACATAGGAGAGAGAATGAAATGAAAAAGAAATTAGTTGTTTTAGGGT
TAGCGGGTGCAATATTTTTATCAGGAATGGGAATCTTAAACGTAAGCGCA
GCAAATTATTCGGATACAAAGTTTAGTTTTACACTAGGTAAACTTGGTGC
AAATGATTACACTGGTTCTAGACAAAAACAGAACACTACATCATCTTATG
TGAAATTAAATTCAATTGGAAAAGGAACAATGGATACATGGCTTTTAAAA
TCTAACGGAGCTAGCGTTCGAAGTAAATATGTGACTGTGAGGCAAGGTGA
AAGCAAAAAAATTGCTAACTACGCTTATGAAGATTATGGCAAATGTAATG
TTAAATTAGCTGCTGAAACTAGTAAAACTCAATTTGTTAGAGTTACTGCT
ACAGGCCTTTGGAGTCCAGATAGTATTTAA
ORF484 (SEQ ID NO:150)
AAATCAATGAAAGCATTAGAACGATTTCTGTCAAAATGGTTATTACCATT
TGCAAAAGTACTAGAAAGTAACACCCAGATGAAGGCAGTACGTCAAGGAA
TGATGGCACTTGTACCAATTACTTTAGTTGGTGCAGTTCCCGTTTTATTC
CAACAGCTAGGCGGGATTCCGAAACTACCTAGTTGGATTGCAGCGGTTTC
CAATTATATCAATAACATCACTTCTCCAATATATTTTGCCACTTTTGGAT
TAATGTCCGTATATGTGGCGGTTTTTGTAGCATATTACTATGCGAAGGAA
CGCAATTTATGGGATATCGGGGCGATTGTAACAGCCTTAATGAGTTTTGT
TGTCGTTGCAGTAAGACCACTTGAATCAGGCGGGAGCGATGTAGCGTATC
TAGCCGGTGAAGGGATTTTTGTTGCTTTAGTAATTAGTTTATTGTCTGTC
GAAATTTTACACATTTTTAAGAATAAGTTGAAATTCACGATTAATTTAGG
ACAAGGTGTTCCAACGCCGATTTTACGTTCTTTTGAAAATTTATGGCCAA
TTTTATTCTCTGTTTTAATTATTGCTATTTTAAGTTTTGGGATAGAAACA
TTAAGTGGTATTCGTGTAGTTGAGTTAATTCAAACACTATTTTCTCCACT
GACATCCCTAGTAAACACATTACCAGGTATTATGTTAATTATCTTTATCC
AACAATTATTATGGTGGTTTGGTATTCATGGCTATTCTGTTATGGCGCCA
GTTTGGCTCAGTGTAGCGTTCCAAAACGTGGATGCGAATGCAGCAGCTCT
TGCAAAAGGAGAACCACTTTCTTCTATGCTGATTTTCACTCCTGACTTTA
TGTGGAGTATTGTAGGTGTAACTGGGGCTGGGGTTACAGGAGCACTCGTT
GTCATTATGATGTTCTCCAAATCAAAACGATATAAAACACTTGGTAGACT
TGCTTTAATTCCAACCTTCTTTAGTATTAATGAGCCAGTAATGTTTGGTG
TTCCAATTGTGTTAAATCCACGCTTCTTTATTCCGATGATGCTTGCACCG
CAAATTGCTGCTTTGATTGGTTGGTTCTCTATCAAGCTTGGATTAATGAA
TCCATTTACGATGGTTTCTCCATATGTACCTGTTCCAATCGGTGCTATTG
TCGCTTCATTTGACTGGAGATATGTTATTGTACTTGGTCTTATCTTGGTC
TGTTCGGCCCTTATTTACTATCCATTCTTTAAAATTGCTGAAAAAGAAGC
TATTTTACAAGAAACGAGTGGAGACCAAGAAGCTAGTTTAGATGATTTTG
ATTTTTAA
ORF485 (SEQ ID NO:151)
GAAAGGACGATAGACATGAAAATTATGTTAGTATGTTTTGGCGGATTATC
CACTTCCATTTTAGTAAAAAAGATGGAAGAGGCGATTGCGGCATCTGAGA
AATTTAAAGATAAAGGAATTACGATTGAAGCTTGGGGGAAAGATGAGTTT
TCTGATCATCTAGATAATGTTTCTATTGTCTTACTTGGCCCTCAACTCTC
AATGGCTTATGAACAGGTAATCGAAGCGACAAAAGAACATGGATTAAATG
TTCCAGTCGAAGTTATTGATAAAGAAGATTATGGCAACATGAATGCAGTA
CCAATTCTAATCGCTGCATTTAAAAAGATTAAAGAAGCTGGAACGAATAC
ATTTAAGACGGAGGGAAACTAA
ORF577 (SEQ ID NO:152)
CACATGAAAATACATGCAAAAGCAAAAAAAATCTTAGTGAGCCTGATAGC
TATTATGTTATTCCTTTCACTAATACCTGGTTATGCACCAATGGCGGAGG
AGACGTCAACTGGGGTAGAAGTACCAGAAAAGAAAGCGGGAGAACAAGCA
CCAACAGAAGTGAAAGAAGAAAGAACAGAAAATGAAGTGGTTTTTAATAA
CCACGACGGGAGCTTTACAAAACAAATCTTTGCGGATTCTATCAATATGG
AAGTTGATGGCGAGATGAAACGCATCGATGCGAATGTGGAGAAAGAAGCA
GATTCTGACATGATTGTTCCAAAACAAACTCCATTAGAACTAGGCTTCTT
AGACAAAATGGAAGACGGCGCATATCAGAAACTAACAAAAGCTGGCGCAG
AAGTTACTTTTCGCTTAAAAGGAGCACGTACAGGCGAAAATGAACAAGCA
GTTACAGACCAACCAGCGACCTACAAAGAAAATGAAGTTACTTATGAAAA
TGTATTTCCTAAAACAGATTTACGTCATTTAACTTTTCCACAATCAGTAA
AAGAAGACATCGTACTACATGAACCTAATCAAGTAGATACGTATGTTTAT
CAAATAGAAACAAAACTTGGTTTAGAGCTGGCAAAGAATGGTGATGTGCT
ATTCAAAAATAAATCCGGTGAAACAATGTATACGCTTCCGAAACCAGTTA
TGACAGATTCCAATGTCGGCGCTGAGACCGGGGAAGCAGCGTTATCCGAG
AATGTTTCCTTTGAAGTGAAACAACTCACCAAAACAGTCTATGAGTTACA
ATTAAAAGTAGATACAGCATGGCTAAATGATGTCGCGCGCGAATATCCTG
TCTATATTGATCCATCTGTCCGGTTAGATGAAGTGTATAATGCCAACATC
AATTCAGCAAAACCAACCGAAACCAATATCGGAAGCAAACTTTGGGATTC
TGGCCAAAATGCTTACACGTTAAAACTTGGTAAATGGGACAATTCAACAG
GAAATAATGCCGCCTATTTAAAAATGGACACGTCTACTTTAAATAAAGCG
ACGATTTCTAAAGCAACGCTAAAAGTGTATAACATTTGGCATATGTCCCC
AACAGTTAAAAATGATCTTTGGTACTATGAATCTAATGCGAACTGGTCCC
CATGGCAAGTGACATGGAATACAGCTCCTGCAACGACACGGATTGGAAGC
GTCAATGTCGGCCGAGGTGAGTGGGCGAATTTAGATGTCACTAAAACAGT
TCAAGCATGGGCAAGTGGCGTGCGACCAAATAACGGTTTCCGTCTAGCTA
CGAACATCGATCAAAACTATTGGAAAAAATTAGTAGCAAGTGAAAATAAT
AAAAACTATCCTTATCTAGAAGTAAATTATACGTATGCTCAACCGGAAAA
ACCTACTGTTAAAACGAATTCAAATGGAGTAGGAACTGGAACAGGTTACA
TGGATTTATCTTGGAAGGCTGTTCCTGGTGCAACGAGTTATAATATCGTT
ATTTCTAATGGATATAACTACGAATATATTAATACAAAAAGCACCGCAAC
CACTTGGAGCACTAAAGGGAAGAAAATTTTCCCAACAAATGACGAAATCG
CTAATGGAGAATTCGAATTCCACCATGATGGCAAAGGAACCGAATTCGCT
CTTGATCCGCGAGCGCAATATGAAAATGCGTTTCAAGCTGGAAGTACTTT
TGGACTGCGCAATTTAACGCGCTATTTATTCAGAGTGCAAGCCGTTTTCC
CGGGTGGAGAAAGCCCGAATTCCGACTTAGTATTTGCCTATATGCCAATC
GAAAAACCACAACCCCCTGCTGCGAAAGCATATTCTAACTTAGCTCATAA
AGAAACAGGATACGTGGAACTTAACTGGGAAAAGAGCCCAATGGCGGATG
GCTATAAAGTACTTGTCTTCAATGGGAAAGCCTACGAAGAATACGATGTT
GGCGCAGAAACAAAGTGGACCACCCAAAATAAAGGGATTTGGCCAACCAA
AGAAGAAATTGCAGAAGGTAAATTCGCTCTTCATCATGACGGAAAAGGCG
ATGAACTAGCAAAAGATCCTTCTCCAGTTTATACCAATTCTGGCGGTAAT
TATAAAGAACGAACAAACTACTGGTTCCGAATTGTTGCGTACCAAAAAGC
GGGAAATAACGCCACAAGTATTCAATCCGAACCAGCGACACCATCCCTTC
CAGAAGTAGTAAATAAACAGCTAGGAATGGTAGATTACTGGACAAGTGTG
CCGGTACGTGGCGGCGAAGTAAACGCTACAAATGGAAACTTTTTATTCCA
TGAAACAGATTTTAACTTAGAAGGCCACGGCCCGAGTATCAATGTCGACC
GTACTTTTAACAGCCAAGATGACGCAACAGGCATTTTTGGTAAAGGCTGG
ACAAGTACGCTTGAAGAGAAACTTATTGAAGAAGAAAACGGAAATATCGT
ATGGGTGGAATCGGACAAGAAAGTCCATCGTTTCACTAAAAAAGGCGATA
AATACGTGGCACCACCAGGCATTTACTCCGAGATTACTAAAAACGCAGAT
GGCTATTTGAAAATAGAAGAAGATAAGTCAGAAACGCGCTTTTTAGCTGA
TGGACGATTAAAATCCGAAAAAGATACAAAAGGCAACGAATTAACTTACG
AATATACTGACGGCAAACTAACAAACATGCGCGACGCTTCCGGACGTACC
GTGACTTTAGCGTATGAAGGCGAGCTCGTAAAAGAACTTGTCGGACCAGA
AGACCGTAAAATCAGCTACACGTATAATGACAAACAAGAGCTAATCAGTT
CATCCACTGCCCGCGGTAAAATATACTGCTATGGCTACACAGATGGCTTA
TTAACATCGATTTACGATCCAAAACATACAGAAGAAAAACCATACGAAAC
AACCTTTGCTTATGAAGAAGAGAAATTAACAGAAATAACCGATCCGGTCG
GCAAAAAAACAACACTTTCTTATGACATGGAAGAGCAACAAACAACTTTA
ACAAACGAGAAAAAGAAGAAAACCATTTATTCCTACAATGATGCTGGAAA
TCCAAAGAAAGAAATTGTGGATGCAGATGGTCTCAAACTCACAACGACCT
ACACGTATGAATCAAATAACCTAGTAAAAGAAGTGAATCCTAAGGGACAA
GAAGAAACATATTCTTACGATGCAGATGGTAATATCACGCAAGCGACAGA
TGCTTACGGTACAGAATCATATACCTACAATGGAAATAACGATGTAACGA
GCGCAACTGACACAGAAGGACGCAAAACGACGGTTACTTATGACGGAGCA
GATGCGGTGTCAGAAACACTTGCGACAGAATCCCAAGTATCCTCTGTAAC
GCAGTATGACGCCTATGGAAATCCAATTCGAGGTAGTGGTGAACTTTCTT
CAGCAGGTAACTTACTTCAAAACAGCGGCTTTGAAAAAGGAGCAGGAGTT
TCCAACTGGACACTGATTCAATCTGATGCAAAAGGAAGCATGACATTCGA
TAGCACTCAGTCAGCTCCAGGAACACTCGGCGGCAGCGGTTCCGTTAAAC
TAACGAGTGAAGCAAATTCTACCGTAAAAGGCTATTCATCCGTTACTCAA
CGCGTTGATGTAGAACCAGAAACAACGTATACATTTAGCGCCTGGATTAA
AACATCCGGAATGACAAACGCCGATGCAATTCTCATTGGACGTTTACAAG
ATGCGAATGCAAAAGATATTACCGATGCTGGCGTATGGCAATCCAACCGT
GCGACATCCATCAAAAAGAACGGCGGCTGGGTAAAACGTCAATTAACCTT
TAAAACATCTAAAAACACGCGCCAAGTCTTGCTTTATTTGGACAACGAAC
AACCAGCTCCGCATAAAGGAAAAGGAACAATTTGGTACGACAATGTTCAA
TTTGAAAAAGGTAGTGTTGCTTCCAGTTACAACCCGGTAGTCAACAGCAG
TTTTGAAGAGCACAATGGAACGCTTCCGACTGGCTGGGCACGTTCAGGAA
ATACCGCACTTACACAAGCAAAAGTAGTCGATAATGAAAGCCATAGCGGT
GATAGCGCTGTTTACTTCGAGCGAAAAGCAACAAGTGAAGCCTACACGCA
TATTGTCCAAGATGTACCAGTAAATCAAAAAGAAGCCAAAGCATTAACAA
TTTCCGCACTATCCAAATCAGAAGACGCCAAAGCAAACGGTTCTGTTGCA
ACGATGTCGAACGATTATTCGATATGGGGAACAATATATTATCAAGATGG
CACAACTTCTTCCGTACAAGGTCAATTCCCACTCGGAACGAACGACTGGA
ACCGAAGTGCCGTAGTTGTTAAACCGACTAAGCCAGTCAAAATGATTAAA
GTTTACACCATGTTCCGCAATGGTTTAACCGGAAAAGCTTGGTTTGACGA
TGTACGTGTCATAGAAGGCGAAGTATTAACAAAAAATGAATACGACGCTT
CCGGCAATTATGTAACAGCTAGCTATGACGAAGAAGGCCGCAAAATCAGC
TTCACTTACGATATTTACGGTAACACTACATCCGAAACAGACGAAAAAGG
CAACAAAAAAACTTTAACCTATGATGCAGACAACGCACTTATAGACACAA
AACTAGCGAACGGCACATCCGTAGCCTATAAGTACGACGACAATGGCAAC
ACCACCGAAAAAAATGTCACCGCATCTGGCAAAACGCAAAAAAATATCTA
TGAATATGACGTAGATAACAAAATTACCGCATTTACCGATGCACTTAATC
GCACAATCAAGTATGAATACGATGCAGCCGGTAATGAAACAAAAGCAATC
ATGCCAAATGGTCGCGTAACCGAAAGCACATACGATTCCGCTGACCGCTT
GGACGGGATTAAATGGAATGATAAACTAGCATTTAAATTCCAATACGATC
CAAACGGCAACCAAACAAAAGTAACCGACGAAATTAACAGCATCGTTACC
GACAAAACCTACGACGATGCCAACCGAATCACCAAAGTAGCCGAACGAGG
TGGTGACGTAAGCTACACTTACAAAGATAAACCAACAAAAGACAACAAAG
GAAAAACAGACAAAGTCGGAGAAGTAGCCATTAACCACGGCGACTACACA
GCAAAAACAAGCTACACATACAACGACTTAGACCGGAATACCCGCGTAAA
CGACGGAAGCAAAAACGCCTATTTCGAGTTTGACGAATTTGGAAACATTA
ACGTCTACACAGCAGGAAACGGCACCGCAGCCAACTACACCTACGATAGC
ACCCAAAAAGTCACCAACGCAGCTATTAGTAGCGCAAGCGGCACCCAAAT
TTTAGACGAAAACTACACCTATGATGCAGCAAGCAACCGTACAAGCATCG
ACAACAAACAAGACGGAAAAACAACCTACGAATACGATGCAGTCAATCAA
CTAACCAAAGAAACACTACCAGACGGCACCGTAAAAGCATACACCTACGA
TGGCTTCGGAAACCGCACACAAGTAGCAATCAGCGGAAGCGAGACAAAAA
CAATTGCCGCAAGTTATAATGATGGTAATCAACTGGTTTCGTGGAACGGA
GAAGCGCTGACGTATGACGCCAATGGTAACCGTACAAGCGATGGCAAGTA
CACGTATACATGGGATACCGGCGACCGTTTAAGCAGCATTACGAAAAAAG
GCGAGAGCAAGCCGTTTACGAGTTATACGTACGATGATGATAACCGCCGC
TTGTCGAAAACAGTCGATGGCGTGACGACGAATTATCATTATGATGGCGA
TAGTATTGATGTTCTGTATGAGACTGATGGTGATGGAAAAGTAGTTCGTC
AGTATGTTTATTCGGATGATAATGTTCGTTTAGCGATGAAGATGAACGGC
AAAACCCTCTATTATCACTATAATGCGCATGGCGACGTAATTGCGCTCAC
AGATGAAGCAGGTAAGATTGTCGCAGAATATGCGTATGATGCTTGGGGAA
ATGTGCTGAAAAACACTGCCTCTACAGAAGAAGCCAAAGCCAATCCGTAT
GGTTATGCAGGATACACATATGACAAGGAAATCGAACAATATTACTTGAT
GGCGCGTTATTACGAACCAGAGCAAGGCGTGTTTACCGCATATGATCCAG
ACCCGGGCGATGAAGACGACCCGCAGACGATGAATGGGTATAATTATGCG
AATAATAATCCGGTGATGATGGTTGACCCTGATGGAAATTGGGCATGGCT
TATCCCAGCTGCTATGGCTGCCGGTAAGTTTATTTATAAGTTTAGAAAGC
CTATTGCAAAATATGGTAAAAAAGGTGCCAAATGGGTAGGGAAGACTGCT
AAAAAGGGCGCTAAATGGGTAGGGAAGACTGCTAAAAAAGGTGCTAAGTG
GGCGCGATCGAGATATAATTCTAAGAAAAAAGAATTTACAGTTACTAAGA
ATTGGAAGGTAGGGCTTCCTAAGAAAAGCCAAGGAGGAAGGATTTTTGCT
ATAATTAACCGTAAAAAAGGGAGGCTATTCGCTTTAGATTACGGAAAAGT
TGGTGGAAATCAAAAAAAATATTTACATGTCCACTGGAACTACAAACCAA
AGCACTATATAATCTATCCAAGAAAAGGAAGGGGAAAATAA
ORF633 (SEQ ID NO:153)
GATGACATGAAAAAATTTATTCTAGCAAGCCTTGCATGTACGACTTTGAT
TAGTTTTAGTCCTTTGGTAGGACAAGTCGAAGTAAGCGCTGCTACAACAA
ACATTGCCACTGCCCCAGAAAAAAATTTACAAGAAGCTCCTCCAGCCACG
GTTGATCAAATTTTCCCAGATGATGCTCTAGCTTTTAAAGTCGCACAGGA
ACTAGGTGTTTCCGAGGATACAGTCGTTACACAAGAACAACTAGATACTA
TCGAAACAATGGTATATGTGGATTTCGGTGTGGAAGATTTAACAGGCATG
GAATATTTACACAACTTAAAATTTGTAGATTTAAGCCAAAATAAAATTAG
CAACTTAGAGAATCTCGCTAATTTAACAGAATTAGAAACGGTATCACTTA
ACTACAATCAAATTACAGATATTACTCCACTAATGAACTCACCTAAATTA
TATAACTTAGAATTAGGCGTTAACCAAATCTCTACTTTACCATCCTTCGA
AAATTTAACTAATTTAAGGATATTAAATCTAAGTAGCAATCAACTAAAAG
ATATTTCGGCCCTAAAAGATACTCCTCAATTAACTAATTTATCTATTTCA
GCAAATAATATATCAGACATCAGTGTCCTTTCAGAATGCGATAATCTCCG
AGTTTTTTATGCCGAAAGCAATCAACTGACTTCTATTGAGTCATTAAGAA
ATAAAACAAAACTAGAATACTTCGATGCTAATTTTAATCAAATTAAAGAT
GTTACACCTCTCAGTACCATTCCAACAATTAGGTCAATAAAAATTGAAGA
AAATCAGATTAGTGATTTCAGCTCATTGGCAGGTCACCGCTTAGAATTGT
TTGATGCTACTGGACAAAATATATACCTTCCAGATGTTGCGCTCGGCGAT
TCCACTAATATTGTAATGAAAGATAATCTCGGAGTAACATTACATGATTG
GGTTTGGTACACCCCAGGAACTTACCAAAATGATACACTTACTTGGGAAA
ATGCAGGCGATAATTCCGCTTACTTTTTAAATAATCAATATCCAACTTAC
CCTTCTGTCACAGTGACTGTCTATCAAACAGTCACTCCTTAA
ORF653 (SEQ ID NO:154)
AAATTAACTACGAAAGAAGGAGGGGAGCTGATGAAAAAGTTTTCCATGCG
AGTGGTCCTTATTATTTCTGTGCTTTTTATTGCTTTTGGGAGCGCTAATG
TTTCTATCGCACAAGAAAGGGACACAACGAATAAGCTTCCAGAAGAGGAG
CTAGGTTCTCTGGATACCTCTAATATCATTGCAGAAGAAGTTGCGCAAGA
GAAACCAGCAGAGGTGGAGCAACTAGAAGAAATCCCAACTACGGATGAAT
TGATGCAAAATCCAGATGTTCTTGAGAAGTCTGTTGCCGATTCGGATGAT
CCGGATTTAACAGTGGTAAGCTCAGGTGATTTTTGGACGATTTATCGTAA
TACTGTGAACAATGAATATAACTTGCGTATGTTTGGTAACGTGCCAAGCA
GTAGACCAACCGCTTGGAATAGTTATTTGAAAAGCATTAAACATATAGAG
ATTGAAGAAGCTACGCTGACAGGAAGTTTTGCATCGTATTTTAGAGGTAC
TGATTTCCCAGTACTTGAAAGTGTGAGAATTGAACAGTGTAATTTGTCTG
GAGTGACGTCTTTTGAAATGGCATTTTATAATTCAGGAATTGAAAAAGTT
ATTATTAGGGATAATGATTATCCGAAAGCACCATCTTTACTCACTGCTAC
GCAATATATGTTTTCCTTATGTCGTAATCTTAGTGAGCTTGATTTGAGTG
GCCTTGATACGAGTGCTGTAACTAATATGTATGCCATGTTTCAAAATTGT
ACTAGCCTTGAAGAGTTAGATCTAAGCAACTTTGATACCAGTTCAGCTAC
TAATATGTATTCCATGTTTTCGGGTTGTAGATCACTTGAGAAATTGGATG
TAAGCAACTTTGATACGAGTTCAGTTGCACATATGCAGTTCATGTTTGAA
AAATGTAGTATCCTTGAAGAATTAGATCTAAGTAATTTTGATACAAGTTC
AGTGACTAATATGTCTTACATGTTTCAACTTTGTCCAGCGTTAAAATCTT
TATATCTGGATAATTTTACGGATGCAGCAATTATGACAGGTATGTTTGCT
GGAACCACCTCCCTAACCTACTTGTTTGTCAGTCACAATTTAAGTACTTT
TAATAACTTAGAAAATACAAGCTGGTATGATGAAAAGAATTGGGTGCAGT
TTTCGAATCTCTCGCAACTACAGACATATCACAGGCAACAAAGTGAACCT
ATTGGCTATAGAAAAGGCGCTTTCCTTTCTCTAACAATGGATGCGATGGG
TGGAGAATTTGAAGATGCGGAAGAACAAAAAGTACAAAATAAAATTTCTG
GGGAGTACTGGGAAGAAGTGATTCCAGTAAAAGAAGGACATTACTTTGAC
GGGTGGTATCTTGATCAAAATTTCACTAATAAGTTTGATTTTTCCTTGCC
TGCTGCTGTATCTACAACTATTTACGCCAAATGGGTAGAAAACTACACGG
TGATTATTCCAGCGTCTATTTCCTTAAATGAAGCTTCCGAATTGAAAGTA
CAAGGGATTAATCGAGGGAGTAAAACTTTATCCGTAGGTTTGAATTATGA
GGAAACAACCATTTCTGAGAGCAACAAGCTAACCTTGTCCAATACGGCAG
ACACAACAGTCCGATGCTTGGCACCAATGAGTTGGGACGGGTCCGAAACT
AATCCGGAAAAAGCGATTTTAACCCTTGCCCCAGGTTCGGAAATAACAGA
AGGCGATGCAGTTATGGAAATAAAATCCCCAGAAAACATCCAGGCTGGAA
AATATACGGGGAACGTCGTATTTTCGATTAAGTATGAATGA
ORF660 (SEQ ID NO:155)
AAAAACATAATAAGGGAGCGAGATAACCATGAAAAAATCATTATAGTACT
AGCATCAGTTATGTTAATGTCTCTTTCGGTTCTGCCACTGGCAACTTATG
CATCAGAAACAGAGAACACTCCTACTGAATCATATGACGGAGAAAATTTT
ATAGCAACCCAAACAGGTAATACTTTAGTTATTGAAGATAAGAAGACGGG
CGAAACTGTAAAAATTGAAATGAATGACGAAGAAAACGGGGTAATTACAT
CTGATGATGGTACAATAGAAAATGTACATAGGGATGAAGAAGGTAACGTT
TATGTAGATAATGAGCTTGAACTAGAAGCACCTCCTTTAGACATTGAAGA
TGGCATAAATATCGCCACTCAGCCTCGGTTACTGAAAGCTTCTAAATGGA
TATATGTTCAAACAACTAAGTATAATACAACCACACAAGGCAATATGAGA
AGTCTTGCATTGGGGATTCTATCATTCATGCCAATTACAGGTCCAATCTT
TGGAATAGTTGCAATTATTGATGCTGCTCGATCAATGGGGGCGAAAACAT
TATATGTTAGAGTGAAACAATATCGTACTAGTGGTTATCAATTTTATAAG
TATGATTCTTATTATTATGCCAATGCTAGTTTAACTAAGCTAGTTAAAAA
AACATCCCAGACTAAACGAATGTGGTAG
ORF72 (SEQ ID NO:156)
GGAGGAATAACTTTGAAACAGTGTCCAGTTTGTGAAAATTATACGATTGA
AGCTAATTACGATATTTGCGAAGTATGCTACTGGGAATATGATGTGGTAG
CTCAAGAGTATCCGGATGAAATTATAGGAGCTAATAATATTTCGTTAAAA
CAAGCAAAAATAAATTATGCGAAATTCTGCGCAGTAGAGGAAAAATATAT
TACACTTGTTCGAAAACCGAGACAGGATGAACTACTGGAGTGA
ORF73 (SEQ ID NO:157)
AAGAAGGAAAGAAGCATGGAATATGAACAAATGGCTGATAGTTTAGCATA
TGGTGAGGAATATAATTTTTACTACAAAAATGAGGAATATTGGTTAAGTA
AAAATCGGGAAGGACATTACTTAACGAAAGTATCCGATGGAGAAACACAA
GAATTTAGGACTTCAGAAGACTTATTAGGAACAGCTAGAATAAATGGAAA
ACTTATAATAGAAATTTGGGAAAATATACAAAGTCAATTCTAA
ORF74 (SEQ ID NO:158)
AAAATAAATGAGGTGAAACAATTGAAAAAATTAACAACAGAACAGTCTTT
TGAATATTACCTTTCTTCGCTATGTATGTTAGGATTGCATACAATTAATT
TATCCGACGAAGAAATTGAATATGAAATATTTGAAGAGTTAGCAATTGAT
TATCCAGCTGCTTTGAGTCCATATACTAGGGAATTGTTGGTGGATAATGA
CATAATTGATAGAGAGCTTTCTTTACTAAGCAAGCAGCTGCAAACTAAAC
TTTTTGAACTTGATGGAGGAATTCTGTGGTAA
ORF757 (SEQ ID NO:159)
GGAGTGTTAATTATGAAAAATACAGTTCATGATAGCGAAATATTGTCTTA
TCATGCAGATTTTGAGAATTCTCAATTAATCATGTTTGTTAAGGATGAAG
AAAACAGAAAATATAAAGTCATATTTGAGGGGCTTTTAACTTTCTGCTTT
GAACACCAAATGTCTAATAGTATCATTTTAGACATAGTAAAAGGAGAGGT
TAGCAGCTTTATAAGTGAGAAGTCAATTTTACTTTCTGAAGGCAAAAACT
ATTTTTGGCCACTAGATTATGAAAGTGAGGACGAACTACTAAATTATTTA
AACGAGAAAAAATTAAATTATTATGAGTTACAAGCTTCATATGGGTTAAA
CGGTTGGATATTATGTTCTCACTATCATATTGAATTATAG
ORF799 (SEQ ID NO:160)
ATGAGACAACCTTTTCAAGTTTTAGTTATACCGTTTATTAAAACGGAAGC
AAATTATCAGTTTGGTGTTTTACATAGAACGGATGCAGATGTTTGGCAGT
TTGTAGCTGGTGGAGGAGAAGATGAAGAGGCTATTTCAGAGACTGCTAAA
CGTGAAAGTATAGAAGAGTTGAATTTGGATGTCGATGTTAAAATGTATAG
CTTGGATTCACATGCATCCATCCCTAATTTTCATTTCTCATTTAACAAAC
CATATGTTGTTCCAGAATATTGTTTTGCCATTGATTTAACGAGTTGTTCA
TATCAGGTTACATTATCTTTAGAGCATAGCGAGTTGCGCTGGGTTTCTTA
CGAGTCTGCTGTTCAGTTATTGGAGTGGGATAGTAATAAAACGGCGCTTT
ATGAGCTAAATGAACGGTTGAAAAACAATGATATGAAGGCAATGTAA
ORF817 (SEQ ID NO:161)
AAAAGAGGGAGCATAATGAAAAAAAGGTTAATAGGTATTATTACAGCATT
ATTGTTAGTAAGTGGGATTTTGCTATCTCCGCAGATGGCTCAGGCGGAAA
CAAAAGTGGATTATGACGCATTGTATCAACAGGGAGTATCTGAAGGGATT
ATTAATAAAGCAGATGTAAGCTTAGAAACATGGATAAAAGAGAATGAAAG
TGAGTATAATCAAGTCTATCAAGATGGTTTAAAAGATGGTGTCTATGATG
CATCATTGTCGTATGAAGAATGGATAAAGTTAAACAACTATGGACAACCT
CCTGTAGTAGATGAAAATTGGGAAGAAGTTCCACAGAAGCCAATGCTTAA
AGGTGTGTATAAAGGTTATAATATTAAAAAAGGGGACATTCTGATTACAA
ATGGAACATCTTCTTCCGGATTATTGGGACATGCTGCTATTGCGAATGGG
AATGAGTATATATTAGATATTCCTGGGGCTGGAAAAACGACAAGGCAAGT
ACCTACAAGTAAATGGATGAGTGATTATGATCCAGAAGGATGGGTAAAAG
TTTATCGCCTGAAGGATAGTTCTGTTGCTAATGCCGCAGCCAATTGGGCG
GACAAAAACTATTATTCCACCAAAGGCACATCCAAACAAAATATTTTCCC
AAAATACGGTATGACAGGAAGTCGATATAGTAAGAACCCTACTTATTGTT
CTAAAATTGTACTTCAAGCCTATTATTTCGGAACAGGAAATAAGCCTGTT
GTGCAAGTATTTCCGTCCTTAGTTACTGTGTATGATTTACCAAACTATTT
TTCAAAAGCATATAAGCCACAACAAGTGAAATACTTTAAATAA
ORF819 (SEQ ID NO:162)
AAAATGATTGGAATGTTCCACTATTTACTTCCCTTAAACCAAATTCCCCA
CAACTTTCATGCGCAACCTGGGCGCATTAGCCAGATGATGCACCAATGGG
ATTTCTTCCACTACTTCAACCGAGTCAGTCACTGTACCGGCCAAGGTTAC
TTATTCTCTCGCTTTTGCTCGCCATTTTGCAGCGCTTCATCCCGCTTGAT
TTGGGAAATAGATTCGCCGATTGCGTTGGCAACTAGGTCGATTTTATCGC
GGAAAATTTGTGCAACAACAGAGGTTTCGTCTGGAATAATTACACTACCG
AACTGTTTTTCATTTTATCAATCGCATCTTCGGTCATTTCAGTAA
ORF949 (SEQ ID NO:163)
ACTTTGAAAAAATTAGTAAAATCGGCGGTTGTTTTTGCAAGCCTTGTTTT
TATTGGCACCTCCGCTACTATGATTACAGAAAAAGCAAGTGCTGCTTCCA
TTGATCCGGTGCAAAAAGCAGATGGTCAAGCTACTTATATCCCCAAAGGA
GTTAGAGATGGGACTGCAACGGAAGAACATGACGGCTTTGAAGATGGAAC
TAATAGCGTACTACAGCAAGTCCCTTTGCTTCGCGCAACAACAGGATACC
CTGATGTTAATGCCTATATTAAATCAAACAAATTTTCAACAGCAAAAATA
GAAAAACAATTAAAAAGCCAATTTCCTAAATTCAACTATCGTAATGGTTA
CGGCAAACCAGAAGGAATAGTTATTCATGAAACAGCAAATAATTCATCTA
CTATTACAGGTGAAATCAATTATATGAGTACCAACTACAACAATGCCTTT
GTTCACGCTTTTGTAGACAAGTCTCGTATAATCCAAATCCATCCTACTGA
AAATGGTGTTTGGGGAGCAGGACAATATGCTAATGCTCGTTTTATCCAAG
TTGAATTAGTTCGTTCGAAGACATTCGATGAATTTGCTCGTTCTATTAAT
AACTATGCCTATTATGCTGCGTACCTTTTAGATCAATATAACCTTCCTGT
TGATAGCGCGCACAGTGACGGCAAAGGAACAGTCTGGTCCCACGATGCAG
TTACACGTTATTTAGGCGGAACAACCCATACTGATCCTGTTTCTTATTTC
AACCAATGGGGTTATAATTTCAATAGTTTTGTTACATTGATTAATGAAAA
GTACAAAGCGATTCAAGCTAGCAAAGTTACTTACGACAAAATCGAATACG
ATAAAGGCGTCACAGCCTACGCTAGAGTTAAAACTGCACCTGGTAATGCA
GTTTGGACAAAACCTTACAGAACAGAAGGCTCTAAACTTGTTAATCAACT
TTCTGTCTACCAAGGTAAAAACATGCGGATCTTGCGTGAAGCTAAAACAC
CAATTACTACTTGGTATCAATTCAGCATTGACGGGAAAGTAATCGGTTGG
GTTGATACTCGTGCACTCGATACGTTCTACAAACAAAGCATGGAACAACC
AGCTAATTTAACTCGCTATGTCGCTTCCAACAAAACTGGTGAGGCCTACT
ACAAAGTACCTGTTGTTGATGCGGATGTCAAATGGGGCACTTTAGCTGCT
TATAAAGATCAAAAACTAACCGTGGATAAACAAGCAACGGTGGAAGGGCA
ACTTTGGTATCGCGTTAGAACAAGCACTACCTTTATTGGTTGGACGAAAG
CTTCCAACTTAACAGCTACCTCTCCATTTGATAAGATTGAATACGATAAA
GGTGTCACAGCCTATGCTAGAGTTAAAACTGCACCTGGTAATGCCGTTTG
GACAAAACCTTACAGAACGGAAGGCTCTAAACTTGTCAATCAACTTTCGG
TCTACCAAGGCAAAAACATGCGGATCTTGCGCGAAGCTAAAACAGTAATT
ACTACCTGGTATCAATTTAGTATTGATGGGAAAGTAATCGGTTGGGTTGA
TACTCGTGCACTCGATACGTTCTATAAGCAAAGCATGGAGAAAGATACTA
ATTTAACTCGCTATGTGATTGCGAACAAAGTAAACGAAGCGTACTACAAA
GTGCCTGTTGTGGATGCGGATGTTAGATGGGGCACTTTAGCTGCTTATAA
AGATCAAAAACTAACCGTGGATAAACAAGCAACGGTCGAAGGACAGCTTT
GGTACCGCGTTAGAACAAGCACTACTTTTATCGGTTGGACAAAAGCTTCG
AACTTAACAACAACTACGCCATACGATAAAATTGAATATGATAAAGGCGC
CACAGCCTATGCTAGAGTAAAAACTGCGCCAGGCAATGCCGTTTGGACAA
AACCTTACAGAACGGAAGGCTCTAAACTTGTTAATCAACTTTCGGTCTAC
CAAGGTAAAAACATGCGAATCTTGCGCGAAGCTAAAACAGTAATTACTAC
CTGGTATCAATTCAGCATTGACGGGAAAGTAATCGGTTGGGTTGATACTC
GTGCACTCGATACGTTCTATAAGCAAAGCATGGAGAAAGATACTAATTTA
ACTCGCTATGTGATTGCGAACAAAGTAAACGAAGCGTACTACAAAGTTCC
TGTTGTAGATGCGGATGTTAGATGGGGCACTTTAGCTGCTTACAAAGGTC
AAAAACTAACCGTGGATAAACAAGCAACGGTCGAAGGGCAACTTTGGTAT
CGAATAAGAACAAGCTCCACCTTTATTGGTTGGACAAAAGCTTCGAACCT
TAGTGCTACTAAATAA

TABLE 4

ORF1025 (SEQ ID NO:164)
VALVMKINKIIEYIVVAIIVLGWTYFIVIKEILPKNIGFISLVILSIILY
FVGKRLNGKHSR
ORF104 (SEQ ID NO:165)
MKKILGGILIIFVIGLFAWRVYDVKANSFSYENKAHAEQEKFQLGSSTIS
AGKAFVVSDADLNKYVTKDYFKQENKTLLLVQLESTEKDIRISDFQLGYK
EFVTLSDTSAASYEFEDGVYKMVLGFNIPKELLATNKTFTLVTPSKYWKN
GARDVVEISL
ORF105 (SEQ ID NO:166)
MNYIKVNHLTKVINNNTVLDDINFELKQGGIYSFIGHNGSGKTMLFRALC
GFIAPTSGEVTINGISISKTRSFPEKTGIIIETPGFLANYTGYKNLEYLA
SIRNQIGEKEILAALEQVGLSGKENLKVKKYSLGMRQRLGIAQAIMEDPD
LLIFDEPTNSLDKAGSQSFIDLILDLKEKGKTILLASHHIADIDGISDEI
FEMEAGQIINRRKV
ORF1063 (SEQ ID NO:167)
MKNLFRLFLVFSIVIIGVVSFQAIDASANETDVYPLPARIIDVFPDENLA
EDMVENFGKKDVTDVITQDDVDAVTSLGLGYFTNYLTDEDLQMLGNAYFT
NVNNIMIYPTQTMFTGFPDLPTLPKLDTLRAEGNLSSEVLPENITVPDYQ
NYPELKYLDLSNRTIVGGLPNFSNIPKLETLLMSSCGLASEDAPDFTNLK
NLQKVNFQTNQFRTEMTDFTHLDSLVSMDLSYNYLNVLPP
ORF1227 (SEQ ID NO:168)
VNITKNGLYIIIGANGVGKTTLAKKILQANRSISCMMKQDDNQILEYETV
LTNISMNEIAEKTVINFLEEHQLDYLITKKSKFLSGGEKRLVNLLRAILS
NQEVLILDEPSNDLDIDVFEKAKQIIYQAAKSKIILLITHDDRFTEYDKK
IEIMKNQSYEADSFSFNKESSKARIIKIKPRRTYFLYIFYLICMMIFAIF
LVILLKTNAEETSPSNEKGTYGLATIYSTNASSYDNNEAINTMLIQSATK
FNKAKFFTEETRINEDEYYEEAINLKKDTYQELIYLELYDPKTEEFINIK
AVMMEALRGDLKLNAETEFISNDENYYKNSDSPSFHVPKSLTLTEIKKAK
IKQLGFELHYSNTLQSNQVEIEFNPSVYAQILKKVNQQDVLITEAYVQLK
AQESFYDFLAENKLYAKKIFIKGYEPELLNAEVNQYSNAVMLIKKVALLI
CLLLLVLLILLIMYEVSYKNSYSTLTYYGYNEKELLQFRKKTYLITNFKI
FSVISTVIFLLIMWSIVHSVLITAIIGVVMIFFFFAYIVIPLIIKNNIRK
AII
ORF1228 (SEQ ID NO:169)
MNKINKFSIFSITKPGIYTITGSNGSGKTTFIENELKNNTNKVKDVAYFA
QKNWKYKTNVEKYLHFPKTNPSLVQKYCELFSVDNYYLEKDIQLLSGGEF
VKVELVRTLALDSSIIILDEPTNNLDNKSSEILANILSELAKTKIIYLVS
HDTRLEHFFDKTIFVDKDRIEVSSNVEIEQNEIQVNSKRVVSNGRILKYL
LSSKFNFLMFAFIIVLTILLTNITSTIILRSVPIEENLTSDYNFELMDIA
ENYSRYFNIEMTESEIEAEFQEPNHLTTNELIELQDKDYIKQIYVVDESY
INEFVLDNSKFEVLALPEIITDSPNYVNAFPVTKMHLTKGRFPKDDAKEI
ALSFAQLKKFFHDDISEESAIGNKLEFEDELYEIVGIVNSPVAAISYSKQ
VSKGTVEVEDKTSEKLNNILLKLEKENYDNPNFSIISIKLASKNQHELLN
YLKVHGPSYQYASNYVDSISQVAFYKQNLAKILLISVIFSLIVSVLIFIF
GRKSFSLINGFLNDMSNLNFTPRKNKRFIYVIMILDFLLSMPACLLVSRV
IIGDNIGMLMILPTLGGSAIIFMLTLLLMSYRDKKNDFRNL
ORF1272 (SEQ ID NO:170)
SRHVINCNFRNLFYFIKSNKTSNKVSEFQYLLYIFCFQWLILLLIRFFYF
SLFYFFQNKVSFTHQTITKAYVLLFVLWIAVFIVCCFRFRKKLRKGDFRK
DSVLQNKRGNLGLNLSKKTYISIGAFFAFILLAQLIGGVMIYVFFISIAV
LCMALSFLGLVIFPEQIFTAYCNFKEFHIEE
ORF1384 (SEQ ID NO:171)
VEPDFAYQFETREEMVEKSSWYAEDFDIVASLEEKYLAGFYQNQVYGYVT
PVEFQEEEMKRAIPSEMFVPLKGLEVSELADWADGFPPELQERYNELAES
VEE
ORF1385 (SEQ ID NO:172)
MSITEEEFEEEQVSELFINYLEKCTPEEIHQVVVEWNFDNPKKPIHWIAN
STKTDKGTALMLWSRTLLINLKREKRWLKKAVGMRKKILILLLVWKKST
ORF1386 (SEQ ID NO:173)
MNEFPVVYEPEYMTIYLLVIIGVFSTTLALSLEKYKFTLAGKALIGMCFI
SLAVLFIYVLELAYLSDIADLLFHTIGVWEVVLFTHPYIFLILAILLGGE
KKPPHVPKLKK
ORF1387 (SEQ ID NO:174)
MLSDITITIPNKTAQTKYDNLAKNMSFKSKEDLESLLDLIKILYINNYYE
EALLCCRLTNDVEFDNDFDVWTFIHRIWMFEMQILVHFGEKEEAEEIATK
IEAHFKIPMKIWNTPEKRYAQYKKNRSRIVLKDLSYEEKINSTPRGESGT
FDWKFVALSSLIKPITLNNMTGVSVEEAEEIFRQYSAELQQTKKYGVSL
ORF1388 (SEQ ID NO:175)
LLYSFQKFDWLIGYSDDGDYVTCVDNGLFFSIDKMVLLDNDINVHLLPVQ
AH
ORF1389 (SEQ ID NO:176)
MKILYYGSPIGEGDCYIVSKNLIGLLVILMMAIMLLA
ORF1390 (SEQ ID NO:177)
LRLTRLKITDLHSSYSYDIEFNKDITFFYGTNGSGKTTILNILSSIVTGK
IYQLFTYEFSSITLHYCFERTKNKEQKIEILTPPPLCIEVTFNGQRYILE
KFNESLSNYRQSINRNLEKDFFGENKFLKEIADMFNYVYLPLNRHISLDN
NMLYDNRLHRERTINAMMHGVEEDENDYYMERDKSMKKVQYLIKTKVMRN
NTRINRLNDNFRNQILRSSIDIHKLTFSIEEIFNEVKNLKVEEIKDIKTS
YINILKNLNQITQIEEKNYVNFFDNYIREIKENENIEDNSVSIQLILKYN
EIAKIKNIVKLAEDMETKKAKVVESVNLFCETINSFISTDGTEKKEILID
ENGGIYLQNVIEKKQLSIYKLSSGEKQIVIFYANLIFGVNENKRGIFIVD
EPELSLHLSWQKLFIEKTIELNKNIQFIFATHSPEIIGKYYNKTFELKKK
IDR
ORF1391 (SEQ ID NO:178)
MNQLQYSEDALKNRYLFFKEINEINIFVEDKGKEYEYEEILSKVFADEYH
IQTIYALGGKPQVLSAFRDCKEHVSSNNNIKNIYLLDGDFDQYLDNIVME
SHPHIIYLQSYNIENYFLNEESVVTFMQGKMKMLKNDVRTKIAYSEWLSN
TLYNIENIFILYCIIQEKELGIPNVGDSEYKVINMIDGSINMTRYEDLKK
QISNCTVNIDLVEQEMKAKIHNINPNVFDLICGKHLICSLYRHLLKITKG
FSYDEFRWHLVQNVEVSRLNFIKERVANL
ORF1400 (SEQ ID NO:179)
MNSDSNNNINKNREAGISIIIPLYNVEEVILETLESIHEQTFDMYEVLLI
DDGSTDKTIEMVTEYISDKPKFQLHTQPNGGPASARNYGLRLANRMYICF
VDSDDIIPNYALQLMYDGAISTGSKLITGATKRFNSEGEWFIPMHIQYNI
AKPGMKTLLKNPELFYSIGPCAKLYHHSLIDGVFFPENIRYGEDQPFVLH
ALLQAENIYTVEKVVYYYRLRDGESQSLTQSVNKDPIRILKSVFQIFDYG
EAELLKNNTEYEIALKYYQRVSSVELWGALRAAIESKKSENQQIAFTMTL
DWLKTKSDDFLNIIPSFRYFLLFSSIERVRYITRDNKENYRQLITYLWER
QGEEAKIAFRKAYPIHMKAALQIMEHNNWGAARKISFKFIIRRKFKAPIL
IRKISRGIIFRLATLMPRKKDQVILATERSTSLEGNLLAIYDYLFYNDMP
QKVYVFLRKNRNWFEMFQLYYALGRTKTIVLDDYYNKIYGLKFNKKTHVV
QSWHATGAFKKFGFSALEGTDANTEEFETRAHSPYTDVLVSSEGIIPEYM
EAFRKQANQIKPIGVPRTDVFFDQEYVAYTKEKYMKMYPQLRDKKVLLYA
PTFRGGPNERFNYSVVLDIAALKKELGDTHILILKFHPVIKNVSFNVDEN
DPFILDLTLNNDINDLMLFSDALITDYSSVIFEFSLMNKPIYFFAYDIDD
YLDERGFYFDYKATIPGEVFKDTPSLIESIKTGKYNYDELEVFKKKFVGS
LDGNSTKRFVETYIVQANEEVKDL
ORF1445 (SEQ ID NO:180)
MKKRIQHLDFYFVGIIVIAIFFNFYGIWNDDTVNPYYTAAVTSMVQNIHN
FFYGAFDPAGFITVDKPPVALWLQAISALIFGIHGWSVILPQALAGVGSV
ILLYVLVKPRFGAWAARITALIMALTPIAVAVTRTNNMDAILVFVLLLAT
FFLFKAVHRAKIGWLLLSFALIGVGFNVKMLQAFMVVPAFLLFYFIATKL
SWRKKLVQLVIALVLMLGVSVSWAVVVDQTAASERTYIGSSQTNSVLELA
FGYNGMERLLGQETGTGSNGNVEMGTPPSTNSTDSSNATPPTPPSGSMQD
GANGGTPPQGNNGTPPTGAPSNAANGGGNGSKMTGSTGMFGTGNAGPLRL
FQTALGDQISWFLPLAIIGMLAIFLVYRNENKRIYQLTSRQKEIVFWAAW
LIPVAGFFSIAGFFHHYYLIMLAPPIALLSGVGLVALFRLYQDKENWQRF
LLPAAVTLTGGLQAFFVAAYLPVLAVVIGIAALITSIILIVLRPQPSKLT
AKITALALAILLIAPTYWSLTPILYGGNSSLPEAGPQLKQSSGGGFADAS
VDSDLISYLQKNNTGETYLFGTTDATTAGPYIIKTKEAVMALGGFNGTDP
TLTVKQLIQMIQAGEIKYFYLPSNSKASDSDVVKWIQENGTEIDSSKWSS
SGSTDDDTTSSASFNMQGGAQGMNGTGTGTLYQL
KORF149 (SEQ ID NO:181)
AFKFYEMPSTDIAYSRTGKITNTSGHAVWTQPCGQINSTLKGPASDYLNK
EITIWRKVENKRGTYYQFSETKTPNIKAWLDARAITLYDQVHFNEEYNQM
AVISTVIGHAVWSTPYLQSDSKLIAPASNYEGKRVEIIRRAKTTRSIYYQ
FSYDNKVIGWLDTRAFSLIPSNTAMVISNSTNDIFSNITDAYNKKTL
ORF1490 (SEQ ID NO:182)
MTKEAEIGKQIFIHVGGMENVSRIAHCMTRVRLGIVDSDLVDVAGLKKVP
GVIGVVEDDTLQIIVGPGVVNKVAGAMAEMAGVKIGETIQENLDSGTKTG
KELVEEKASKTKAELKAKQNNSSGFKRLLKSISNIFVPLIPGFVGAGLIA
GIAAIISNNITAGNLDAAVWTQYIDILGVINKGIFAFLAIYVGINTANEF
GGTPVLGGGIAGITLLSGLAEGHTITNIFTGDPIVAGQGGIIGVLLAVWL
MCVLEKNLRKIIPNAIDIIFTPTLVLLIIGLVTIFLIMPFAGLVSDGLVN
GINWVIEVGGVFAGFVLGTLFLPMVMFGLHQVLTPIHVEMIAQNGYTILL
PILAMAGGGQVGAAIALWIRCRKNKPLVNMIKGGLPVGILGIGEPLIYGV
TIPLGKPFLTACLGGGIGGAVIGYFGNVGAIAIGPSGVALIPLIANNEWL
SYVIGLIAAYLGGFILTYFFGTPKDAMNSVEL
ORF1491 (SEQ ID NO:183)
MLENLATEERNEKTIDLDTLSPKEILAVMNEEDLTVPIAIKKVLPQIELI
VSGVISCFQKGGRLIYLGAGTSGRLGVLDAAECVPTFGVSKEQVIGLIAG
GEKAFVAAIEFAEDSKILGENDLKQILKTANDFVIGIAASGRTPYVIGAL
DYAKSSVGAKTGAISCNANAKISAHADIAVEVVTGAEILTGSTRLKAGTA
QKLVLNMISTASMVGIGKVYKNLMVDVLPTNKKLEERSKRIIMEATEADY
ETANKFYEAAEKHVKVAIVMILTNSTKEIALEKLSEAKGFVRNTIQK
ORF1492 (SEQ ID NO:184)
MSNGSIINRIEGILNDLPKSEKKIGQAVLANPEFTTTASIHKIAQKADAS
GAAVIRFCKSIGLQSFPELKRQLSLDLAQPQKKGYYDIEPNEDFHTITEK
LVSNMIQTMNDTASQLDEAKVLEACELLGEADTIYTYGVGASWLVAEDIS
QKWLRAGKHVLATQDAHVLAMAFATGKKKAVFIAISNSGETSEVLQLVDQ
AKLNNVIVISLTRFGSNKLKEKADLSLETSRAPEAEIRSTATSSRQAQLL
VIDILFYYYASHHYDEMIQQIKHSREATNRFRE
ORF150 (SEQ ID NO:185)
MLKRNVQKGMISLIAIMMFLSMFSFTNLNSIKTVEAATTAYNTIEYNKSV
NLDANIAYPETNAFWSAPYYSEGSTFISSATAPSYAKKDVKLIREAKTER
GIYYQVKLGDKIIGWLDK
ORF1504 (SEQ ID NO:186)
MQKAIKIMLVLFLMTTVFLPFSNVRAASTDVVNIPDPYLNEGLKSIVGNP
FLTELTEANLETISVADISYMNGVPGYAVTGLISDLTGLEKAVNMTKLYF
SNQTEIKNLNQIKDLPNLKKIVGVTTGLNDIKALGEMPALEELELGGDYI
TDFTPLLEKDNLKSFSYNSYAWLNPAYHQIDNEEFKKFTNLKSLESLDVT
WNNITDLSSLTANDHITNLNLSYNKFTNIAPIATMKELKVLYLNNNNLTS
IDSLNTLRGLTIAYADNNNITDLSNLKDFFEGMDVVGDYKGLQVNNQTIT
LPTINIKEGGTAISNNPTLDIDGEKMPVSSISDGGTVSTDNKTVSFTNLP
VGNKTVTYKATFTATSTKGVPLSYSIKVSQPINVSAQSDSTVNVFYKDEN
GDELAPSETISGKSGENYQTIEKTITNYTLKEIEGQPsGQFGDSDATVTY
VYEKADGAPVTVKYVDVDGNELATSDTLNGKIDAPYQSTAKSITGWTVKT
TPANATGVFTNANQTVTYVYEKADGAPVTVKYVDADGNELATSDTLNGKI
DAPYQTTAKSLSGWAVKTTPTNATGVFTNANQTVTYVYEKADGAPVTVKY
VDVDGNELATSDTLNGKIDAPYQSTAKSITGWTVKTTPANATGVFTNANQ
TVTYVYEKADGAPVTVKYVDADGNELATSDTLNGKIDAPYQSTAKSITGW
AVKTTPANATGVFTNANQTVTYVYEKADGAPVTVKYVDVDGNELATPDTL
NGKLDTSYAATAKNLSGWKLTATPANANGVFTTDAQTVTFVYAKQEDNPK
KEDKNKTPIKISENKPTASKVTRIKKQTKLPKTGDNQQDSILFGLIGTCF
VLLGIYSISKKNS
ORF1590 (SEQ ID NO:187)
MKRKKTALRILVTLAVVMAISFWVGTSSKEEVQAAVIDQPTPINEIFTDE
NLANAIKATLNKPSTTSDVSQAELDSISEVTAESSNIASLEGAQYLNNID
TLILNNNKITDLNSLAGLSKLRILEANDNQLSDISALANVTSLHQLRIAG
NQIKQLNGVSNLINLETIELSNNQITDISPVSGLKNLIGLGIDYNDISDL
SPIAGLAKLSHLTADNNQISDLSPISSLGAMEIMRLDKNQISDVTPIANL
ANLSYVFLAENQISDISSLQPLFNSPNFFGITLDNQKITSEPVLYQQELV
VPNNIKDEMGALISPDTISDNGVYASPNIKWNLANYTNQVSYTFNKQLAQ
GSFSGTVTQPLHNAYTATFDVDGVKTNEVVEETKLLQEPTAPTKEGYTFT
GWYDAKTGGNKWDFATDKMPAEDITLYAQFTINSYTATFDIDGKLTTQKV
TYQSLLEEPAAPTKDGYTFKGWYDAKTGGTKWDFATGKMPAGNITLYAQF
TKNATPDSNDPTTVTPTGNGNGTSTPSNSGGNTTLPTAGDENTMLPIFVG
VFLLGTATLILRKTIKVK
ORF1654 (SEQ ID NO:188)
MKKQIITIAGGGSTYTPGIIQAVLNGQERLPVSEIRLYDIEATRNENMYL
ILDYMLEQQGFSSIKLVQTTNPEEAFTGADFIFSQIRVGGLKMRDKDEKI
PLKHGLVGQETCGLGGFSYGLRSIGPLLELVGFIQKYAPEAWILNYTNPE
TIISEAVRRQFPGIRMINVCDMTIGIEDTLAKNYGYDRDNWIATYYGLNH
FGWYTKIYDKSLKRDIMPELLEKLKVQEIKEEDPSWNRAFNNIRFNVQNF
PDNLPNNYLEYYLYPDMYVDYADPNYTRANTVMDGREQKTQEMADKIRNK
EKGDVLDFYFGVHGLYIVDIATSLLNDEKSRFMLIVENKGSIPNLRSDAV
VEVPAYVGATGVEAIALPAIGDFHKGMIVIEAQVAAEKLLVDAYFEKSYQ
KALQAFTLNQSVPNATVAKKVLDEMMIENKAFWPELK
ORF1655 (SEQ ID NO:189)
MLNQRQKNILSTLYNENNWLLGKKLADLFQISDRTIRNDIRVIKESIGDD
FIFTSKKLGYAYNMEKPFPIDVEAETGFEQNRMAQLIQQLLVEEGVDIYE
YGAETFTSESTIQRDIQWLRGYFEQLLGLDVVIHSSDGVYAISASPTTKM
ELLNRIATLDEGLKTNLLTNCFPEINHEKIRQILLEMIHQHKIVLKYFDE
TILLAQLIYGSAFFQKHAEQQTSTKIANPFLKQLFDTIQQEMGYQISAEM
KQFIVSEYEKIVANNHFENQVTTKMVLEGELYQEILLILEEIKHVYLIDF
TADLDVTSDMTKHIFIALERAKRGIVIKNQVTHIITQQYSYLLDIAIFIG
EKLLERLGVILNQEEIILLVMYLYQYYRKIEAKHQLNQVVRIALIVLEGK
AANYYLREQLAEVLRPVNAEVIEITDNSQCQLLLAENIDVDLCISTKKID
LPAEVPCIILANNIGLIEEVTIKKQLSATVEANKMKKFAYIKEKYLHEEL
FLTDYAYEQKYNAIEFLSQYCIDKEYVPERFTEKLYNREQLFSTAIPTGI
AIPHPIKNVARKSGIFICILKKPCAWDTHKVSLIMIPMIEELDGTEAPLI
NDFLSLIASNKSYVEQISACSTYRECVELLQTIYNNNE
ORF1680 (SEQ ID NO:190)
MEQIMTLLQSIKLALKQLLSTKFRTFLTMLGIIIGVFSVILLVSIGEAIS
KNVSTQLGDMGSNLLTVNFYSDNPNDKFTYKDAKKLLNEDEIGSPEVMQT
KEVRTSEKTASNQVAGINEYYASIKNLELASGRFCSNVDVIYAQKVTIIG
SEIAKTYFKEQNPIGEYLQIAGARYMVIGVLKEKGESLFGSADKKLFIPI
SSAERLFKTDTVDLYYIQTKKVEQVPAAVKVVKKKMRQFFPNNEDAYTVV
NQQQALDTFDSITGTLTIGLGAIAGISLLVGGIGIMNIMLVSVSERTREI
GIRKAIGASSGNILMQFLIEAVVLSLVGGCIGILLGIFSAQIVTTTSSFE
MYVSASTILLAVGFSMCIGIVFGVIPAQKASKKKPIDALRAD
ORF1681 (SEQ ID NO:191)
LIQLFNISKSYQMGECTIKALDNVSLQIDQGEFLAIIGPSGSGKSTLMNI
LGILDRATIGEYYLNKTNLMRISDKKISRIRNRKIGFIFQQFNLMPRLTA
FENVELPLVYRGVGKATRKKVVLKSLERVGLLDKEKHIPAQLSGGQQQRI
AIARAIAGSPELILADEPTGALDSKTGEEVMTLLREIHREGNTLIMITHD
KEIANQAERIIEIKDGKLREWNKL
ORF1683 (SEQ ID NO:192)
LKKISWVIIMLVIVAASVGYYFIKENERKTPQTIDYKTVETKRTDLSVYV
SAEGHIVKKVNEWPDYEDFAVKIMVDELEINQIKEKQTADVYVEAVNNKV
YKGKVVDINEKGIINGSVTSYAVTIDLEDETKVKENMSVSADVLVALEKK
ILTIPIEAVNTDKADKHYVYTVDANKQKKKIWIEVGKHNTKSIQVVKGLT
EKQLVIIP
ORF1684 (SEQ ID NO:193)
MPKIAKGNRLENVAFEYIKNKITTGEYPTGYRVVEAKLSQELNMSRTPIR
RAIINLCHSGFLVHQYNRGAFVQNTEVTITEFFSRMKLVELLMYESTEKL
ILREDYIVVDDIIEIAEKVIQYEKNKEYELMRDTFEDFIVAFIGKLNNDY
FNRIIQELWNGINDNATKEVRLIIVSASDRIAEELANIIEILKSWNYGEL
KKCFQHIMNAMILIAF
ORF1717 (SEQ ID NO:194)
MKKFSMRVVLIISVLFIALGNANVSIAQERDTTNKLPEEELGSLDTSNLI
AEEVAQDKPAEVENLEEIPTTDELMQNPDVREQSVADSDDPDLTAVSSGD
YWTIYYNSANDEYSLRMFGNVPSSKPSAWNSYLKYIKHIEIEEATLTGDF
SSYFRSNVFTVLESVRIERSNLSGVTSFARAFEGDSESESPLEKVIIRDN
YYPETPSLTNISRMFTLCRKLSELDVSGLNTSSVTKMDTIFSNANSLKEL
DVSHFDTSSVTDMSSMFAACNSLEELDVSNFDTSSVTNMKYMLSGLHLKK
LDVSNFDTSSVTNMYGMFAYCYNLEELDVSNFDTSSVNNMLHMFYVCNNL
EELDLSNFDTSSVTNMFAMFAYCTSLKEIDVSNFDTSSVTTMSAMFFECS
SLEALDLSNFDTSSVTTMASMFENSTALKSLYLDNFTDAASMTDMFKGTT
SLTYLFVSRNLSTFNRLENTSWYDEKNWVQFSNLSQLQTYHRKQSEPTGY
RKGAFLSLTMDAMGGEFEDAEEQKVQNKVSGEYWEEIVPVKEDYYFDGWY
LDQNFTNKFDFSLPAAVSTTIYAKWVENYTVIIPASISLNEATELKVEGI
NRGSKALSVGLNPLATSVSESNELTLANTADTTVQCSAPLSWDGSENNPE
KAILTIAPGSEITEGDAVMAIEAPENIQAGKYTGNLVFSINYE
ORF1718 (SEQ ID NO:195)
MHSKEVFLMREYLDSKSQKKVALLEKIFYAENHTSTQEELLNDLNITYPT
LISTIKTINFDIERFGYKAFSIVHSAPNLSYTLKISDNCSIQLIINAYIR
ESPKFQILETLLLASFPNLQALAKKVHVSYSGIKKEIKELNEELSERNLY
ISTGNQVEITGDEFSLRIFYAFLFLVAYSGDRWPFSFVRYDEITDLLESC
PKEIYRANSIDKAMMIHYYVAMHLLRDRMNCQIDTTRQFKVALYKACTEE
SKKSESAFIKKVAKQLPNRNYKEMTYTTQIILSTIVAFGSYSSIEKMPSF
FYMDEQLEEMGFMKLVDFASERVNDNLSIPFSEKEMELLRYSFASINYRY
FLLDNLINKFNNIVPGYTDLDRNIRKIHKVNHLEPLISQLVNLKEMDPLK
PFEERLTSDYLIILDKRIDFSIHTLPIKVTILSTISNETAVFDFMRYFSS
YYNLEIINQVDPVVDLYISDFSVSPEVLTSLRINQPIIYVNTRWLESDYV
KINDNLAKIARKNLSLIKKIRETLVGMDMENV
ORF1739 (SEQ ID NO:196)
MKFAIIMPFYNAEKRLALSIDSIIKQSYSFLKHVEVLLINDGSTDGSGAI
ANRYATKYPNNIRVLTVPNGGPAKARNIGIHNVSEDTDFVGFLDADDIMS
ENNLASIVTFLNESNVSMLVPAFYYLDDFGSKKKISPHKLNYRFANGNRV
ADIEKEPEAIHFYIGGTFLRYDCLKEFTFDESLYFAEDQLLITQFLLKYR
SYGLIADAGYYYYRDLKQKGSLVSSSWKKPERYTPFLQKVYQTYLTDSKE
IYGKVIPYVKYLIAYHAKLFFYKENTYFREVLSESEQAIFVQELQKILQE
VGASTIMELDTPLVVKEMMCSILQNGWPLQFETAEKQDIPLVTVKENYRI
GKTVAIELLLEEADIHDVKWVARTSFKEMPARLVKRKENQTIWDVVVREK
GTVEKAVFKLKPYQTKARLFYQDEEKDTPIADINIISSILGKLKRNRALK
RKFKQGGVS
ORF1813 (SEQ ID NO:197)
MWVLMKNGIVDVEATNAYNAAVLNGELPHESNEAQEEAELLQAVIQSVKE
GIDPVTGQEISKAQGFSIISGFIFYYTSGGYKGKKLKIPKKLLNNIKNRR
SKAPKLSTLSEKEQLKLANKYKKKAPIAIPDNAKIKAQTKKAGYEQITYK
WNDGQFKYEVRWHTRTPEAPLEQGNTNVVQRTVPGNGGDKPQTFFKIGDD
EWVEGYKWYDAINARKAGNATPEQIKILDKGHWKE
ORF1849 (SEQ ID NO:198)
MKSKGRLFLYVVLALSIVIGTNVFIKIDAHAAAAPPAAISQIFPDDALAT
EIQTTLGKSSTAEVVTQTDLDTINSLTLTSKGISSLEGMNYLTNLGTLIL
TGNQVSDISPLKGLTNLTMLQLSGNPISDISALSNLKNLQALDINDAQVT
DITPLSGLTNLKGLGLYNNQLENLSGVNSLHQLRSLNVSNNKLTNLDELQ
ALSNLSVLYANENQINNLQGLSNLNNLFLLDLSANQIVDTTPLAGLTKVQ
TLYVSNNQISDVTGLSSLINLDWLDISQNKISNIRPLNSLTKLTIIQMTN
QLIVNEPISFESTVTIPNLIKNIAEQTIDPDTISDNGVYANEAVTWNLPT
YIPKVSYTFIERDTIGNATGNFSGTVEQPLVQYFKATFNIDGQETTENVE
TGTLLQEPPTPVKEGYTFNGWYDAETGGTKWDFTADTMPANDITLYAQFS
INSYTATFDVDGVISTQAVEYQGLLEEPPAPTKDGYTFKGWYDAKTGGTK
WDFTNNQMPANDITLYAQFSKDASSGGDGGGTDEGGGNSENSTEGAPNTS
DIDTINHIVLPATGDDHVLFPIFIGTFLTSLALLTLRRK
ORF1912 (SEQ ID NO:199)
MSRIDIAELNGFLQGLRSSNAEAKKMIRGIQQAASKYAQDKSLKGQAVSA
SQSYFASSYPSIAQSILEALEESEERLAQYIREFGSQVDSSPSARIDAEI
LQEAMAKVASLKRKEEDLHRQLTAPNTKPDMQQVYVVQARSAHTQLLQAI
EKEDILERYIAFEQSHAQFFSALTELIHSTGRAVQELKQNVTFHEKTGTY
AVPKSVHASISLMRKAMDKARKENAKDSFPEAFEDYQLFAYTYVNDKGET
VTMWLLERNGKRASNKELQAFLEENGAELDPILYTNLSGDELERKVNDAW
KDGVNYLNGQKVTGFSEATLRSSAYVASVKDAMDDAGLTDMALGLGFGIA
AARNKKIIAKKSEKVEVDLGRYSVATLNRDQARLVVEHLLERGEISLKDL
GDIIPKGTPNTFKTTDTLKNGAKYEFQLADGQKAIIRWHEPDPIAAAKFP
GSASGSRWTAQIKIGSKQVTINKGWTKKQNSNEVHIPIKGR
ORF1913 (SEQ ID NO:200)
MDSTHTRLEQQLQQVKKAQDVLQDNLGQTKRKQVEQEWLEEDSHQLEMEK
QGLLDFLRGGWQGEEANGFHRYLEEQQHEEAMAWRKDLSEKRVHLEEEAR
TTRAENHDIETKQASLRKEWNQ
ORF1998 (SEQ ID NO:201)
MKKLEKSVPVIRFKGFSEAWEQRKLGEIANSFEYGLNASSKTYDGENKYI
RITDIDESSHVFNQDNLTSPDISLDNLNHYLLEEGDILLARTGASTGKSY
CYNKIDGKVFFAGFLIRAKIKHEYNVSFIFQSTLTERYNNFIQVTSQRSG
QPGINAQEYARFALYIPKLKEQQKIGDFFKQLDDTIALHQRKLDTLKQMK
KGLLQQMFPKSEEDVPKIRFADFDEEWYQRKLGEISDKVIEKNKESTYFE
TLTNSAEYGIISQREFFNKDISNEKNLNGYYIVRENDFVYNPRISNYAPV
GPIKRNKLGRIGIVSPLYYVFRTFDTNQSFLEYYFDGTVWHNFMLLNGDS
GARADRFAIKDSVLKEMPIPYSTLYEQEKISFFLDEITIIINLHQNKLKK
LSSLKKAYLQNMFI
ORF1999 (SEQ ID NO:202)
MALSTEQKTKMWAMLNQTRGQIGLTAYKDYIFGILFYKYLSEKATHWLNG
VLRGENWESVYSQDSVKALNYMKKNLGYAIQPNEFFVDWKKAIDTDRFNI
GMMTDTFTHFNQQIAFEAKNDFEGIFDGMRFDSADLGANAQARASVMISM
IELLSSPEFDLSGSNDTVSDIYEYLVAQFATVLASDMGQYYTPKEISNVM
ARILTFGREDMEKFSIFDPTVGSGSLLLTTASYMKNSGRRGVIKYYGQEK
DATPYRLSRNNLMMHGIEYNDININHADTLESDWPDGVVDGKDTPRNFDA
VMANPPYSAHWNNKDREDDPRWREYGVSPKTKADYAFLLHCLYHLEDNGR
MAIILPHGVLFRGASEGRIRKALIDKHQIEAIIGFPEKLFLNTPIPVCVV
ILRKNRIESDVLFVDASKGFEKIKKQNNLRSEDVEKIVDTVINRKEIEKY
SHVATLDEIKENDYNLNIPRYVDTFEEEEAIDLVALGNEMVALNADIKKA
ETDFLGLLDELAVTADTKEIIEATKAVFR
ORF2013 (SEQ ID NO:203)
MKSKTKQIIMIGVVLFQSLFAYPLITMAEENESKSVNTETTLEPKVALEE
KTPQKPTLTNNLKQEKTVLQAGETYETVFPDAALATVIAKAATGSEDITQ
EVSQTDLNKITSLTATSKGIVDLTGIDLLSKLTSLSISGNQITDISALNG
LVNLSNLNVSNNKITSFNLNANSNLPMLSAVDIRSNNLKNINVQDQPKLR
TIECDTGSSSELTEVTLKNLPILIVAGNGSSAYQNDIVFSSTPGLSKVIL
ENLPSISSSVRLDRCAIEELVINNLPKVSMVNISNNKITTLEGLENLSAV
NTLYVSENLVTEIENLHAFPKLQTLTVDNNHISVLPTSLKTENPVLTTLS
AMNQTITLKQKVIVSDLVLDNEVKNFGQITTAKSISNKGTYQNNQIKWLF
EDIKSVNAVDYQFSEPVQEATIQGTFSGKVTQPIKASKVPVISADAEMNY
PKNETVSEAAFFKDISASVTDDATLTSDFESVVDFAKAGTYEVTLNAVNE
DGVKATSVTVLVHIAKSPAPVITADKEITYTKNAEVSITEYLAAIHAKTN
DGSPIESDFATAVNWGTAGDYTVTLRSTNEDGVEAIPVEVTVHIAKSPAP
VITADKEITYAKNAEVSITEYLAAIHAKTSDGSSIEADLDTAVTWGTVGG
YTVTLRSTNEDGVEAIPVEVTVHIAKSPAPVITADKEITYAKNAEVSITE
FLAAIHAKTSDGSPIESDFATAVIWSTAGDYTVTLKSTNEDGVEAIPVEV
KVHIVEPIAPTISNVTFDVDDVQTTESLEAGELISEPLSPTKEGYTFIGW
YDSKTGGNKWDFTTDKNPAYNIILYAQFSKDTNKAEAAGGDKPSTPSSIK
VSPTGQSESGNLENRSNIKLPATGDDNATVLLVGFGLLMLGLFIRLTQKK
RAK
ORF2017 (SEQ ID NO:204)
MIKSLYIRRSLMMLITSLLVVSQLNLTVFQAFAEEKGEEPLSYEVQEELS
KDKKKAKLKIKATSNNNQVEILSIETPDGNKIEGKEAEYTAEKNGDVNFI
ISYKENIEGKETEIKTFKASYEVKNLIIEKEEANQSKDSVKQNETKAPLK
NNQLKSGNDATVTLNIPAYDGTAWANGDIKDVNVTVDFSNSTSTSKEVEF
TLPDGMRFVSIPVPSSFQVPTGVDSGVINRLSAGDPIGTAITSVTIPNKE
TTYDKATFGTVNYKLTPGTEKVSFNFSVRVDANKYYGATDLKAPIKVEAF
VGDNKMAIASEEKAIRAEGNKVVGYANQTHVSTMFRNWYKSFRTPDVLAS
TDDEDSYAYTKSYSVVNGMNQVDERGTNIFSPKNVSTTLYYPEGMEYVGV
VNEKYALLNENANTTITHYPEENKVVFDFKQQNFKGVTDTVYAIKYKIPK
GTPAGTYTAPKVPHAVITTYDDKVFESDALTNNSTDLTTKTTLDACRVVD
STENKMALIVGNKNINPDNETWAGNVRINNKDTAGTKTNQVFQIEFDSNW
EASIVNIPFDGNRKDNDITNIQYKTNFDDTYQTYNGNLPKNSMKNIAILE
ADAVGLQPGEYFTEVKANVGDFSTGYTSYSESGGFGEESTNSYGIVKPGI
SSVQFKVSIWDEADEVNTKVSGTSVYSVADNITTAANGTANFYNEQGQVI
KTAGAGDKFTTKATLVLFNYPYGTRTVLNDPEVYLRQLDGTKILPSSIKL
TDQDGEEIEFSIKIETAKNGEKVYVLKTKDATVGRYVGYPSKHKFLNISY
DTTIDVTLDKSLHTDIQQLLAWGGPNVKAATAANTFMDTGLDVNQNGIEN
ERLLSANSSTLSVPKQDAVTVETFLNLAGEGAKASYVEGDDSTVSYFTPG
TEADYTVRVTNTSTGIASAFELYIPIPKTGQNFGSKFQTETFKWDMKLND
AVQLTDEQKAQFDISYATEANKNNYESESVYSAVPSDYGKVNNVRIKVKT
KIDPGETQTIKVPLKVDETFNSATEGNKISERDVYNPYYRVTTNAYSGTL
PGTKVGAELIILEASGFLFQDKDANGLYEKAQGDTALANETVELYKWNEN
TSNYEPFLKNGEPVSVKTNADGKYTFNYNLNLSYGKYAVKFPERAGNQFT
LKQVGQDNTINSTVSNKGTDKGWVKEIDPAQPVSQNINAGYMEYAPDNDL
KVNLSAKIVQAGKSLKVTLPKVRATSGEAVEDTIEPSFFHNIQAITDGYK
WTSNDTTLATTQTANDGSGIIVGISAGNKAIATTDIGITIKDIFDTEKKS
TAPVYITEPSATIDQKEGLILGALNFSLEYKDSNKLTETEAITLAKTASF
EEVKTGVLSTAQDCTSSVKVDAKQLKAIQEGSNQGGTYPLTYQVAKNGKT
AEVVIQVKVEKDLTTVNVHDSILYLGDTWTATDNFDSALNKVGETVPFGD
IQVEGSVDTNTAGIYSVTYTYKGVSKKAKIEVKENLTEINAHDSTIYTGD
TWRAGDNFDSGLDKDGNALSLKDLTVIGTVNTNLAGVYTITYKYEDTVSS
ITVTVKENKKGINGEDSSIYVGEAWTAADNFDNAVDKDGKPVSFADIKVK
EEPKVDVNKAGRYQITYNYDGASTTVTLTVKEIKTAINAHDSILYIDDNW
SAKDNFDSARDKDGNIVSFNDVQVLGTVDTSQAGTYPITYVYAGITKTIQ
VIVKHPKTAVRAHDSVIYVGDNWKAQDNWDNTLDKAGQKVKWKDITVKEN
PAVDVTKPGVYEVTYSYDGISATINVTVKPRKTTVKIHDSSFYAGNSWNA
KDNFDHATNKAGEKVAFKDITVAGNVDSKTPGTYEISYVYDGVKAVAKVT
VLKNHSILMVKDRVIKVGEKWNAKDSFIQATRRDGKNIPFSQVKVQGKVN
LNKAGKYQVIYSVDPNEGTEDAGKQMLSVTATIQVENRGDAGLQDNEPRS
VKDNNQRAANSNDIKLIPKTGDQTNQWVLWAGLCLTGLANLLWGFALRRR
KVK
ORF2019 (SEQ ID NO:205)
MKKFSMRVVLIISVLFIALGNANVSIAQERDTTNKLPEEELGSLDTSNLI
AEEVAQEKPAEVENLEEIPTTDELMQNPEVLEKSVADSDDPDLTVVNSGA
YWTLYYNSANGEYSLRNFGNVPSSIPSAWKSYLGNIKHIEIEEATLTGSF
EAYFKSKIDGFRVLESVRIERSNLSGVTSFREAFFSAGIEKVIIRDNDYP
TAPSLLTTESMFENCYNLTEFDVSGLDTSAVTNNKKMFYNCRTLEELDLS
NLDTSSVNSMNSMFGYCESLEKLNVSNFDTSSVNDMYHMFRDCKALEKLD
VSNFDTSSVTNMGGMFVNSTSLKELDVSNFDTSSVTNMSALFSSCRALEK
LDVSNFDTSSVTTMLANFVACNSLEELDVSNFDTSSVTTMQSMFFECSSL
EELDVSNFDTSSVTTMQSMFEKCTSLEELDLSTFDTSSVTNMQSMFINCA
VLKSLYLDNFTTAKTMTDMFTGTTSLAYLFVSHNLQSFYGLANTNWYDEK
NWVQFSNLSQLQTYHRNQSEPTGYRKGTFLSLTMDAMGGQFDDAEEQKVQ
SKISGEYWEEVIPVKEGHYFDGWYLDQNFTNKFDFSLPATVSATLYAKWV
ENYTVIIPASISLNEATELKVEGINRGSKTLSVGLNRTATSVSESNKLTL
ANTADTTIQCLAPLSWDGSENNPKNAILTIAPGSEITEGDAVMAIEAPEN
IQAGTYTGNLVFSINYE
ORF2074 (SEQ ID NO:206)
VEITETKEWWKESVVYQIYPRSFQDSNGDGIGDIRGIIERLPYLKDLGIN
VIWLCPVYKSPMDDGGYDISDYYEIDPMFGTMSDMDELIEKAEKLGIKIL
MDLVVNHTSDEHEWFEKAIADPKSKYRDYYIFREGVNGNPPNNWRSYFGG
SAWEAVPGEENMFYLHAFSKKQPDLNWENIVVRNECIQMINWWLEKGLGG
FRIDAILNLKKRIEYGTFRADGEDGLVFIGHWILNQPGIEEWLKEIDERT
FKKHNAFTVAEADVPEERLSEFIGENGHFRMVFDFSYTDIDTPETGEWFK
NSEWTVKELKEKIITNELVTQRNGWGAKYLENHDQPRSINKYLPQEYQDD
RSKKMLGTLFMMLHGTPFIYQGQEIGMSNTRMESIDDYNDIATHDQYHRA
ILSGMSPEEALEGMYRRSRDNSRTPMQWNNQKNAGFSDSDEIWLKANPNY
LDINVEQEQIDDNSVLNFYKKLIHLRSDSSKYKEVAVYGELLPVESSDEV
IAYKRKTDDAELLIIVNFSDSENQLCIEGTYEQVLANVALPEMVENVLEI
PAYTGAVFSRVLEVD
ORF2075 (SEQ ID NO:207)
MKRATVWPNLLLIVLGCIWIFPIIWIVLTAFRAESGQFVSYIFPKEYTLD
HFAYLFENHENFPFMLWVKNTMIVAVCSCLLSTFITVSMAYVLSRLRFRF
KKTMLKTALVLNMFPAFMSMIAVYYILKAFGLTQSLTALVLIYSSTAALT
FYIAKGFFDTIPKSLDESAMMDGATKLSIFTKITLPMSKPIIVYTALMAF
MLPWMDFIFAKVIMGDNVPKYTVSIGLYSMLNQTTANTMYTTFAAGCILI
AVPITILFIYLQKYYVEGITSGAVKG
ORF2076 (SEQ ID NO:208)
MMVKKNYFNIFKNGSVSTRLSYVIMGAGNLAHKQIAKGLLFLFSELAFLF
FLVFYGITLIQGMATLGTVNQSWNFDKSLGIMVRTPGDNSMLMLIYGIMT
VVICVLFLFLYLANIRSACQVEGFQRENKKIPTFKEDLKSLLDNKFHVTL
LTIPMIGVVVFTIMPLVYMISLAFTNYDHEHLPPRNLFGWVGFVNFKNVL
NGDISSTFFPVLGWTLIWALLATATCFFFGVLLALLINHKGVKFKKFWRT
IFVITMAVPPFVSLLVMQNLLHAAGPLNTMLLNWGIIAEPIPFLTDALLA
KVSVIFVNMWIGIPVTMLIATGILMNLPKDQLEAARMDGGNSFHLFRYIT
FPQILTVMLPSLIQQFIGNINNFNVIYLLTGGGPSNSNFYGAGDTDLLVT
WLYKLTVEAADYNLASVIGIVTFILSAAFSLFAYTRTNSYKEGSN
ORF2110 (SEQ ID NO:209)
MKKLRIFTLMLAFALFLLGGGIVAQAAEEAPIDEKIVGETVTNDGEEFIV
DEISDLEDVNSTTGTLDTSDEVEVDLSGLTIENEEAVLTPGLKTIFGGDG
RKLVTNTTQYPYSTSAYLVMEFPNGKTYIGSGQLIGEDSVLTAAHCLYGK
KDGGWAKKVTVYPGYNGTKAPFGTAKARKMYVPKEWTKKEPSTEDYGVIK
LDKNIGTKTGTMGLTTNTSGAITISGYHGDKKGKLYTQTGNISQVTANNV
FYRLDTTGGSSGSGVYNSKKQILAVNAYEYLNGTGDNFGTRITKEKLNNI
YTWAFDNNLSVSKQKGINYELHVQSKGWMGNVANSMTSGTVGLGLRAEAM
KISLSGMPYSGDIQYRSWIQGSGWQGWLKNGEISGTRGQSKRLEAFQVKL
TGNNAKNYSVQYRAHVQNKGWQSWVKDGATAGTTGESLRIEAVQMRLVAK
ORF2192 (SEQ ID NO:210)
VAEAKFEAALIKKLETEGWTYREDLSNVSIKKLEEHWRDILNETNAHKLN
GVPLSEIEFGLILQELQRIKTPYDAQLLLVGAGGVGSIPITRDDGSSLEV
EIFYEDDVAGGRSRYEIVNQVRFDNLPKGLTTKRIIDVALLINGIPVVHI
EEKDEHLQNQWRAFEQLKGYHGDGLYKGLFAFVQVQVIMSQHSAHYFARP
NAFEQYNKTFVFGWRDENNKDITDAFEFIHQVMSIPALHRLVTVNMIPDA
SNDNIMVMRSYQIQATREILQRMKEMEASGLVQKEGGYIWHTTGSGKTVT
SFKVAQLLASAPRIKNVLFIVDRIDLIDQTLENFKSFAYAHFKNRIKKVN
GRELKRELKRKGSSQILLISVQGLTKAVKKGLENDDWNVIIMDEAHRSAS
GESVKLIKKALKKTTWFGFTGTPNFYSDEINDVKTTREISTHDIFGKRLH
TYTIKDAIGDGNVLGFDVTYFKPHWVVEHPQEDFSEKLYEKEVYQSDVYR
QEVVKDILDNWTKTSSGALIAGKREENAFQAMFAVSGKQAAVNYYNIFKS
KGSHLNVAMTFSRDESNEHGTKEQNEALKNAIKDYTEKFNVASILDAKDP
ARAYMLDITKRLARKRPYNQGKEEDRLDLVIVSDQLLTGFDSKFINVIYM
DKMLKEGMLIQAISRTNRTFDLNSKPHGKVRFYRQGDEMREFVENALRIY
TRGGNDTLQEAEEETKNQLPKDLENDDILAKPQSHQINELEEAILRLKEL
AGDDFSQVPRGQNDLKEFVGLALTTQTKIQQLIQQGYELGSEIEELNEQG
EATGRMVRLNISSIDEFGALQARLYDAKEKLPPEERPDLTEIKIGIEFYH
HEIIDYDMLVELLNTFMDEKNKSNKDAIEKHIVPMDEESQHEIHGIVDDI
EAGHITEHFTTDSLQETRKKYRTEHRELKVRRWAANQKVNGNRIVEAFDL
FLPGHTLIDNPKLADIVREIEEEENIGFFEASDFEEELMAFFNSL
ORF2217 (SEQ ID NO:211)
MIRVVDLFSGAGGLTLGFQNRIINNTFLPMSDYNILFANEVDKHASEAFS
LNFPQIPMLNCSITELTEDYLDNHEIEYSNIDLVIGGPPCQSFSTVGRRQ
YDEKATMYKEYRRMLSFLQPKVFLFENVTGLLTMKNDEGEPVLEDIRKEF
GDFSDFEIDLTYEIRETVLNAKHFGVPQNRERVFLLGIRKDLEIKQNWEF
PERLLFEDDFLTLEDAIGDLPALRNGERKLAYDGDPYTPYQNLMRNNSTI
LMDHFNGLNGEKMLRIMETVIPGEGKKYINELVESGQLDSKYYLTSGYNN
TYSKLWWDRPSSTITNNLSTPSSFRCIHPIQNRALTPREGARIQSFPDTF
QFVGSKEKINSQIGNAVPPLLAIVIAQEIYNFFEENNWI
ORF2247 (SEQ ID NO:212)
VSRIDIAELNDFLHGLRSSNAEAKAMIRKIKEAAMDYAQDNRLKGEAVST
SKRYFSSTYTSICQSIIEALDESEERLAQYIREFGSQVDSSPSARIDAEI
LQEAMAKVSQLQRKEEDLHRQLTAPNTKPDMQQVYAVKSRSVHTQLLKAI
EKENILERYLAFEQSHGQFFSALDELIQATGRAVQELLQHVTFNDKTGTY
SVPKSAVNSLLLMKKALDNARTENDQDPFPKAFEDYTVLAYTYVNDQGET
VTMWLLEKDGKRVENKELQDFLEKHGQELDPLLYTNLSGEELERKVNDSW
KEGINYLNGQKVSGFSGATLRSSAYVASLKDWTDDAGLTDMALGLGFGIA
AARNKAIIPKKSSGLKFDDIEKLENHFTKHGGEFKGAYSNINEYLKGANN
VIKNGEKVQYNYTLKDGTTELRTGYVKFMGNTSKGKAKFEFVGTNLSGDI
TTYHVKRGEDVYKLLNGNKHINVINPIE
ORF2291 (SEQ ID NO:213)
MKKFSMRVVLIISVLFIAFGSANVSIAQEMDTTNKLPEEELGSLYTSNLI
TEEVAQDKPAEVEQLEEIPTTDELMQNPDVLEQPVADSDDPDLTVVSSGA
YWTIYRNTVNGEYSLHMFGNVPSSRPTAWNSYLKSIKHIEIEEATLTGNF
ASYFDNSAFPALESVRIEQCNLSGVTSFASAFYSSGIEKVIIRDNDYPTA
PSLLTTQSMFGYANKLTELDVSGLDTSAVTNMQNNFQYCRALEELDVSHF
DTSSVTTMRGMFQYCELLDKLDVSNWDTSSVTTMMSAFAECNSLEILDVS
NFDTSSVTDMTAMFQNCYALEKLNISNFDTSSVTKMYANFSGLYEVGKLD
ASNFDTSLVTTMNRMFQNCKSLKELDIGNFNTSLVTDMDRMFINCAALKS
LYLDNFTTAKTMTDMFTGTTSLTYLFVSHNLSTFTGLENTSWYDEKNWVQ
FSNLSQLQTYHRKQSEPTGYRKGEFLSLTMDAMGGEFEDAEEQKVQSKIS
GEYWEEVIPVKEGHYFDGWYLDQNFTNKFDFSLPAAVSTTIYAKWIENYT
VIIPASISLNETSELKVEGINRGDKNLSVGLNRTATSISESNKLTLSNTA
DATVQCLVPLSWDGSENNPENAILTLAPGSEITEGDAVMEIKSPENIQAG
KYTGNVVFSIKYD
ORF2293 (SEQ ID NO:214)
MKKKRVVIISLLLLLVSVIGISSYFLFKDKINLLDVDHSAVDNNGKKQKD
TSGEENTIAIPGFEKVTLYANETTQAVNFHNPEINDCYFKISLIHPDGSV
LWISDLIEPGKGMYSIELEKTLAVGEYENAVLKYECFSLNDQSPLNGSEI
NLKLVVV
ORF2336 (SEQ ID NO:215)
LKKRWIIALAIIVITIFGLGVKFYMDEEKLNKEMMNVVYSDEAKQVFEKR
LTNLDPNAFTEKGIIHSYKIEEGSVEHNPMGGIEVGLIINNDSELNVSYT
LSKNNGELSGGASVVSEKLSKLLGRWEE
ORF2372 (SEQ ID NO:216)
MANLKVIENEATILASIPKHNVDYNARVMQSVSVDYERDMIYWTQQYSGK
KMTDAGAGESYNITRTDLKGNYIDQMWCLNGGHGTNIALDYNLESKKMHI
WSAYKINNKWEVVCYPYESNKILKGTESSIFISKVESGSYNRISGDLKND
MLVFHSGGDPKTFNIRIVRASSVREGKLEVLYKVKATEANDAYVYQGCAL
DFPYLYTASGTGGVEEPKQLTCVDIVTGKRVYQTTFKFNTKANQTTESNF
AEPENVCVYYKNNQKHIVVGYALGGAGNRNNRAFDLVENNSENIETEIES
LRNLIISRKRTEVIFDQSTKGDMTTNFKLRETLNNFDIIQVVLESGGGYT
TASRLVSPKLFEASKSFIFASSNIGDASGNNVDMYEYAANFNDDLTSFKN
DRAVKIEVSNNGTVRGNITNNGIKKIYGIVL
ORF2375 (SEQ ID NO:217)
MKKFSMRVVLIISVLFITFGSANVSTAQERDTANKLPEVELGSLDTSNII
AEEVTQEKPAEVEQLEEIPTTDELMQNPNVLEKSVADSDDPDLTVVSSGA
YWTIYYNSANGEYSLRMFGNVPSSKPTAWNSYLSNIKHIElEEATLTGSF
EAYFRDNVFTVLESVRIEQCNLSGVTSFRSAFEGQLALEKVIIRDNYYPT
TPTLRTTENMFLGCYNLREFDVSGLDTRAVTNMRAMFQNCTSLEELDVSN
FDTSSVTTMQNMFQNCTSLEELDVSNFDTRSVTSMAYMFYRSAGLKELDV
SNFNTSSVTAIQGMFYDCSSLEELDVSNFDTRSVTSMTFMFLNCANLEEL
DVSNFNTRSVTSMTYMFGGCTSLKELDLSNFNTSSVTAMYNNFQNSTSLE
ELDLSNFNTSSVTNMSYMFQNCTALKSLYLDNFTTPKTMTDMFIGTTSLT
YLFVSHNFKDFNGVENTNWFDEKNGVQFSNLSQLQTYHQQQREPTGYRKG
VFLSLTMDAMGGQFEDAEEQKVQNKVSGEYWEEVVPVKEGHYFDGWYLDR
NFTNKFDFSLPVAVSTTIYAKWVENYTVVIPASISLNEATELKVEGINRG
SKTLSVGLNYEETTISESNKLTLANTALTTVQCLAPLSWDGSETNPEKVI
LTLAPGSEITEGDAVMAIEAPENIQAGTYTGNLVFSINYE
ORF2440 (SEQ ID NO:218)
LHNIFFLITLFPGMLLLLTKWIPVLSRKSTFFQYLLCLFLITIMNSLFFR
QQFVVVLSLICILFLPFILFFVEYIFVERQWKKLLTIYKKNKIIIQSIVW
FPVLEEIIFRFFIYQYCELFDFSNIQYILLATFSFVIAHIFYQGVSSIVK
ILFSFILSILFLLTLNIFLTIIIHCIFNFLVYIVRTSKYENHRNW
ORF2462 (SEQ ID NO:219)
VEKPFLTVVVPCYNEEEVLSESVTQLTNIIEKLVMSESISDKSQIMFVDD
GSKDRTWELIQQYSESNEHVSGLKLSRNYGHQGALLAGLTEAHAYSDCVV
SIDADLQDDVNAIIEFIEKYHEGYDVVYGVRDKRDTDTYFKRNSALAFYR
IMSKLGVNNVPNHADYRLLSKRALTEFLRYKEENNFIRGIVPLLGFKSTK
VFYNRNERFAGESKYPLKKMVLFAVDGITSFSVAPIRLLLVLGSVIFMIG
VVMGIYAIVQKIIGAVVPGWTSLIVSLWLIGGIQLIGIGVLGEYIGKIFK
QVKERPRFTIEENVFETKCKENKISER
ORF2541 (SEQ ID NO:220)
LKKFVLIFICFLTLTTIVPWNTLETKAASTSWLEQELDGNEAFITETERV
LSKNREDITLADLETIQELDIYGDASSIPDKISDYKNLNTLLALNGTISE
IPTSITKLTKLTRINVDNNNFQEFPMILLQMPSLSSIEINRNKIKEIPSE
ITTLSPHLGSLDVRYNELITLPDNIFTTEWESKLSLLTTGNQLVSDIPAD
WLDNFNQADNMLEFYNNPPNDYHQKQDQLTYSGARIEVPLNTDLKTLTPD
KTKLGLKSGRTLFEQHEFMYYDDGTSNNILTNGVATATGNGYITIKSTLS
TNSNPFAKVRVPITVTPPVKGGDVTVQYKDTTGVVLADSITLSGNVGENY
TTTAKTIDGYSLTTTPTNANGTFSTNPQTVTYTYKKDPIAQPVTVNYIDT
DGKTIAPTETLSGNVGENYTTTAKTIDGYSLTTTPANANGTFSTNPQTVT
YTYTKDPIAQPVTVNYIDTDGKTIAPSETLTGNISENYTTTAKTIDGYSL
TTTPANAKGTFSTEPQIINYIYAKNAETAQPITVNYRNSTGQKIAKSEVL
TGNIGESYSTQPKTIAGYTLTTIPPNAKGTFTTNAQTVTYMYTPIAISAL
PVTVNYLDENGKEIADSVVLNGRVGEAYNTLAKEIDGYTLIKTPTNSNGV
FSAEAQSIDYIYRKNKPVVIVNPPIKNPIIKIDSSTNVIKEETSVLPKTG
DSNPYNDFLAGILLLSSAMFLWKNHK
ORF2568 (SEQ ID NO:221)
MKLGKVGALLLGTALVFQAPFQVFATSVNGEEQVKKEAAAPKISVNKQTL
AATSDLEQTVGEAITKTIHLQDQALPNNEWGSYISDVKVTLSNMDGISYD
VKYGPLSEDGTHYQYADITLSGAPTKAGTGSISLEYYDGAGNGGVYSYSV
NTKSKTTIQYVDGDGNKLAEDTVKTGDLNTAYTSEPKTIDGYEVDETKLP
SNQNGQFAETNQTVTYIYSKTKSEVNKGTVGISFYTPDGSRRELKSSLDL
SYAYPDGVPTDTVTFGDLAKNTTYKDLRNTPDAPTLLWNDLLHYMVDYMN
GDIDAAQFEEAVGLTPANFDLDFISRNFEGYKFDEAMYQENLSKLVTFEQ
DGDQVDLQVPFKKVEVGADITVKYVDTAGNELASVETLSGDVDDNYTSEA
KTIDGWTLKETPNNATGVFSKEAQTVTYVYEKNDDTDVTPAPDNSADTND
DSNSSDNTATTDDNSSSAVKEKAQTQASSPKAGKVTTQESTSNKVTAQSK
ESLPKTGDNVLESSLLVGLGMLLLGGLFVFLRKTRKVK
ORF2646 (SEQ ID NO:222)
MRPTNVYTPEDIYLKDRTGFNNGNIAYQYSIYRALWNDDVEIHADGLSSN
PNLAEKINENYDLYVMPLADAFRDDFRPVLRNYTQLIRKLKIPVIVTGVG
LRANYEPQLDEGFSFDEDVTNFVKAVLEKSAQIGVRGQITADYLKKLGFN
AELDFRVIGCPSLYTFGREIKIRDFHLTDQSTIAINASPTSSEIAINFLN
NMITTYKDYHFIPQHLDEFHLMYAGGPDISSDINGYPTNIQHKYYQEGRV
KYFTSMPSWFDFVKKVDFSIGSRLHGNVIPTIVGTPNISFVQDARMRELA
SYHALPHVTIDELEKTNNLQELLTKVDLKSAEKVQARNFDNYIDFLDTNG
LNHIYKYDKNRKSAPMDELINSITFPTSPDPISILNPQEMLNRVKISTNL
LKERHDFSTRYRVNLVNNQLTQLKKTTSEQNKKYQQKITETQEKNQQLEK
QLTDTKQKLQLTINKNHELTNKIRHYQGTLNRKSVKTTLKVANSLANLKK
KVSRS
ORF2666 (SEQ ID NO:223)
MEMKKLIKCTAPIFIATLIILPVSPSISALANETSEISEGINATEKTIET
EELVVEEISNDEYQVLDKETNETTSIEFSKNHMQSTITPNDGTIDTMIKK
DNLIYLNGEVIGEEVKEESRQLLKASAYKYVTTFKTKMSLKKTSASIAVS
LAGLLGGPVGVFSTVAGMLLTLKSYAPSKEVYIKIKQYYNSYSREIKNDY
SIYKKSNYTGLLKTFTHKYRPYG
ORF2758 (SEQ ID NO:224)
MVIVLLYNAKRIETVFLCLLLSENAVGNQAFAIDFGKIEDIDVAFKNTKR
YKLFPSCTDTFKENYFLGSFSNMKSAFFLSK
ORF2759 (SEQ ID NO:225)
LQIKNKANLITYSDSLGKNMEELSKVMETYFEDAVGGIHLLPFFPSTGDR
GFAPSDYTTVDSDLGSWEIIEKLGEKYYLMFDFMINHISRESLFFQDFKK
EHLNSKYKDMFIRINDFFPPGRPNEKDLDLIYKRKDKAPFQEVEFADGET
ELVWNTFGEEQIDLDVTAEVTKEFIRQTIKNMAAHGCSILRLDAFAYAIK
KLDTNDFFVEPEIWDLLDEVKAEAAKYDMELLPEIHEHYSIQMKIANHDY
YIYDFALPMVMLYSLYSGRVERLANWLEMSPMKQFTTLDTHDGIGVVDAP
LLLTDEELDYTSAELYKIGANVKKIYSSEKYNNLDIYQINSTYYSALGDD
DKSYLLARVIQCFAPGIPQIYYVGLLAGKNDIDLLEETKEGRNINRHYYT
IDEIKNEVKRPVVKALCNLLRFRNTSEAFDLEGSIEIETPSSNEIVIIRK
NKTNKITATLKANLSTKTFQISENERNILI
ORF2763 (SEQ ID NO:226)
LKKILHIVIALSVICSIILVPVDWSAKAGGNNKRDDLIKSVSFYNSNGQN
VTAKENYNEKLNYFLEVAFGGNSFQKGDYFNITLSSDALLYTEKAYDLKV
DIDPTAVTNEQVVGKVTVEKINGSPTLHFVFTEDSESFFINSFDASFKIQ
VMPAHGDKNVINLSYSGAAKNFKNIGTSSVEWNVNMADDWPPVGISDFSK
ISGDLYHAILVYEKPSSKVNYETEILVSYPLFEKRIPLGNVQNIKIEVWD
EAKEIYRVGVAGVDYGTITYDVGTPFVGGPIFQMNCTIPFKGISTKTRVS
FDIDTNVDGKPGTTDPYLVSLSSVSSATKSLEFYPVNNADTKMTAAFFGK
VTTRFEDELGNPVTFDNYSTVTTPGKVNQDGKFEIAEPFLHNSVQNVDKH
AYDTLLDTNKYKLLEVTSPNKLSETADNLSIQIKRGYQNDVLYKIKALQK
PVISALPEIEYSKTVNRTMEEFLEDVEAKTDIPADIDCDLTNVKWGVPGD
YLVLITAVNEDNQAADPVPVTIKISKNPAPVITVDPEIIYDKTVTKEEST
LLNEVNARTNDGSTITSNINDKVKWGVPGDYEVTLNAVNEDGVAAESKTF
IVRILKSPAPIITVDPEITYPKTITKTEAELLQEVNAQTNDGSPLVSDMN
DKVKWGVPGDYEVTLNAINEDGVAAEAKTFIVRILKSPAPIITVDPEITY
DSSVIKDERELLKDVHARSSDGSVITSDSQVKVKWKKTGSYTVTLNAVNE
DGISANSVQFTVHIVDAKAIPVVIEEKPESTPKPDTKEKVVIKKEKLPKT
GDTNTKTILSGIFCLGAWYLLRRK
ORF2777 (SEQ ID NO:227)
MKNQFTYLINNKTTQGMFILILFIPCIEIVQLYIMLKPDAVNIHPAFAFF
LAGSSRGHITQILLLWFLTVLSLLLGADSPIQEYQTGVRNIIINKIGKKA
YILQKLAVSFILCFITMLTTLLLNFILVSIVFLGGTYKMGLNGLGSLNTL
FDFSIQNPYLADFGFGFMACLMAGMAGLIATSSSLFFLNKKFAYPAAFFI
WFLMILPNNSIMFIFQPFTEYGFEIILPIFLVFSLIVLIIVGVLYLYEVK
YVKE
ORF2778 (SEQ ID NO:228)
MLKSRYFLLIFSMFLFFSVFWFTQNNEYLSFPKNRELVLIMNGSLYGYIS
IKSLCLMLVFPYLIFLLLFSKKEQIVALAREKNRLRFYHKILKDTVIATV
LFVGLYLSVNLLYSFIFLSNKLLTATHFYSGIFFTSCYLFSFI
ORF2779 (SEQ ID NO:229)
WNFFYFLLLVLFYLAIGFLFRIIYDLTTSTGQALIFGAFVICIVYLIDWI
ILEGIYWTPLHNLNFFDLWLQNGSISSDIPFILIPNAAVAFILYLISSNT
FIKKDFY
ORF2780 (SEQ ID NO:230)
MKNNKLIILVVICLFLQAILFMAFDFPFKTLPILDGFPVGLATPVVTRLL
LYWYLPIIAFSFYISGNLKDLLSSYGFLQISRNYKKEYWLMKQFLKLLIK
VILFTSLQLALIFIFTPYSIYSTAPFLYLILGYSIMLFTIFSLQYLLELF
IDAQKALLLINGYVIISILMADLIYQNTTVTWPYYLLLPNYGMGFRTGLI
PFTNTSTLLISVPTSLIILLVVLLCVFIVAIKKFKTTDIL
ORF2815 (SEQ ID NO:231)
MNEKQNEIPFDFSYYALELLGKGLYKNRWSAISELIANGIDARATKISLY
MNLIDKEKAVIEIFDNGTGMDYDDLVSKYVHIGRNKRDEELDDVERNALM
GRKGIGKLAALNLSQKYYLISKTRNESSLWCLDATEVNKSDTPKLKRVES
KSVALESIEHWKENSTGTMIKLTNVDMTGFGIQSMEGLKLKLSDFYLLNQ
MSCEIEVAYITTKEEKNNIKFKKVEKKVAFKNFYGFFENMENDKYKASLA
LTVRFPSVYETITEKPRKLYFDKQNFPEIKGKRRFKNKNGTLSEKEYEFE
LKGWIGIHTSTKITDDAERNDITFFRNNTYTPNKLRLYIRDKLIVEDFMA
QYIRSTQATSGYIEGEISFDILDVNDLEDITTSDRQGFTHEDDRVKLLID
ILKPIVNLLIRERNKMGGQIRKEEEEYREQEREEIRKQKLVEAIKRKEAE
DQKEAAEKAKAKVNQENNILKNRITQKDIHLGSEKKRNIFLKSSLSEDKK
SFSQKARMIRINVKTIENTTSFLVNEITKEKPKFNIIKEKLKIISHNTNR
IKRIISYVDSAKFNIDNEKTEGDLIGFFEEYVVNIANQEWEKPQGKVVNP
GKCSLIKVFSPQDIGVLIDNLFSNSSKNKARNIIISIKEDKKYCYLNFKD
DGNGLPSHITDTAELFEFGKSYTIRGTGVGLAHIKDIVNDMDGKVYIPKD
NKEGFEVEMRLTK
ORF2817 (SEQ ID NO:232)
MNKETERKLAASLTSQSIEILPFIPYFLQDFFELGSSPKDITYLIKQHMP
LSAESNFLDLACGKGAVSIGIAKELGNPVKGIDLIPAFIEEAKCKAKEAQ
VESLCQFEVGDVNKSVQNEEKYDAVIFGAAADILGNPAETLEKLQGTVKE
GGYIIIDEAYVPELAHNNQVKYQNYEYLTRKEWLNLFEQNQLQLVEELEG
TAEVDFELEKQHLLARANELIQQYPEKKALFEGYLKSQWSEYDDLEEYLI
AVTWILHKK
ORF2819 (SEQ ID NO:233)
MPKLVTGYERQQTKNLIIEHTSHLIYIKKGIQGFTVEDITRAARIGKRKF
YTCFPSKEACLFEVVEYSYQAQLEAFKKIMEEKGSLKSKMTRFLKEVYLS
EKSINNYFSPEDFHAILQKLPPTYTEREERMTSEVLETANTYIDLTPAQW
EALVMLLDCLTYTATRSYVETAKKAKEETLDILIHSIADYVEKQTQC
ORF2836 (SEQ ID NO:234)
MKYMVKWRGFFIVAIIGLLVFQNVSPVLATIVDEKTTMITLKIIKEDKDT
KEKINGSSFEIKNKKTGETKEVSITEHGTIIENSLSEGEYIVKEKKAAPG
YTLDEQTYNVTLADKEEAITSSSTKKEAEKTPSVTEQPSKKGNLKAVITD
NIFTAVKVENGTGNELGATNRIKNGGAVVLKMNFTFSGKNYKAGDTFKTV
LPDSFNFGTTNLTGDFLPSTEAKWDLNASTRELTITFFKDGVQEGNYDIE
LSTALKSFSETEKTSQVAVFNTAGGNTVYQLEIIPEVDKATQVMLEAMPS
KVNPDKATVDARFNLTKETSELGELRLSDTAYGGSTIINRNSIKVYSTDI
SAKGTFIGSKQLLTENTDYELIYAPSGLTIKLKEGLKAKGYQVTYERSID
KTNSSLSTIGTSATTVGSSGMLSNGSMTISVTIKAYDHLIKKAVYNPVTQ
CIDWTINVNYDLANLTPGTVLTDVLTDDNVSYVADSLKIKRVTFNEESGE
AVIGDDASNDWTVSTISDNGSFNNNYKKTDEKAYQVTYSTKLTDFSPRKI
KNEVTDEKGVKATENFDFKPDLLNKEAGEIDYYNNTMDWTITVNSEGINM
QNINIVDEFSTGVKSLVSYNVYAYPSDSGYKLLTEGRDFTIQKDVSPAGF
KIKLIGNYATTDNKIVVKMKTKIDLTDGAKTLDNKASFSYFDGSLTQYSE
TVKAEATPETSILANGGKVGKWNPATGEINWIVSVNAMGKKYDKLVLDDE
FLDGTTFVEGSLQYRNVVNSSELTDLSIPLEIKGTLAQVGDANYPTKIDT
SANKIHLEFGNLDTNRVFVKYKTKPKDNWFFSQWVNNKAIVSDNGADEQI
YETKEFAFLQNEVIKVAGNIDNVYGNKVNWNMELLNISPERTLSNPVITN
RLEQGNTGAQFIKNSFQVINTKTNEPINEENYDITFEGNTFTIQFKNYTA
MAPIKVSYSTISLLSGPISNETTVEAEDFSNVPMFFKKPNAAVSPVFTVG
SGSGIATIGTIKITKVDEDDTTKKLEGAKFQLYTLDGEKSGQEIKTNSEG
EILLDGIQSGKYKLVETEAPEGYNISDEYKEGKEITVNSSGEELLLTIKN
ANKKGKVILTKKDSASDEVLADAEFELQNAAGSKLKEKLTTAASGNIEIT
DLAPGDYKLIETKAPAGYQLDATPVHFTIDFNQSEAAKVSKTNTAKTGTV
VLTKKDSATNTELADATFELRNEDGALVRENLVTDDNGEISVADLAPGDY
KLIETKAPTGYQLDAAPVHFTIDFNQTEAANVTKTNKKKIGTIIVKFIDV
EGNQLNDEEMHTGNVDEEYNVKAKEIVGYTLVKDSANKKGMYKETSQEIT
FVYEKKANPIIVEPTEPSKPTEQLTESATVAEPKPIKQNFKTTNKSTNNK
RKLPSTGDEFPYTMLFIGLFVSVAGVFFLKKPKQIK
ORF2847 (SEQ ID NO:235)
MLDSTKLDSTKLDSTKYKTKNYLNFDYRVKIENVESYVTDHSKIGNHSFL
PLIRYVSSFEKRIEEKNPEFDNRPIKTKDRVIMYAGHMDNFIYKYYAEVL
NKDFYNKFCNEKGIDDCVSAYRNNKVGKSNIDFAAEIINQMVNYKEAYIL
VGDFTNYFDKINHELLKKHLAEVLNQPRLSKDWFNYFRSITKYGYYEKSF
LNEEYGSDESIKRSNKKSYFENISKFREFQKNNKTLCNKNKFGIPQGSAI
SAVFANIYASEFDLKLKEIADEFSGIYRRYSDDFILVIPKSDIVNEQKIR
RIETDTRRVASEYKIELHKDKTGLYLYENDKIEDIISNEVSHLDYLGFVF
DGTTVGIRGKSPYKFYYNAKKLITFAQKVKVKKELTDLPYKKKIYGLCTD
LGKNYNNHGNFISYAKRAQKKFDEISPNTNNLIMNQLKNRKKKIEKMLGY
KIHTKI
ORF2848 (SEQ ID NO:236)
MKLIITLIIILSNWLIQELLMILEVNLKQFLTDNADKVLLDCLKHHILYS
KNTGMLILSQNKHFLKKTMYNFNYLSPTLNKYYISYTKRALS
ORF2854 (SEQ ID NO:237)
MKFYIKEIQLTDSNTWKLQGFSEGKINSIQAYYNEIREYKHPEQKLNIAF
TQDKNSFTATISVDELASLSLPNNQTVWKFKVNNDYPYTHLITDGPIINK
PFQPENSLYKYHFDFPEGILTLVSKPIELLASIEEYKLDSDVMSGSIKIK
SPLPSNQFNAKLIFKRRPTPSFYLFEEQQQSFDLGLITENIVNFSIPTKD
LSTAFLVDNTNILDAIIEVSSSHNKTGLSAFISIDADMKPAIPREIKIAA
PLFATLRSYITGSNRLSFYFKKNIQGLVSLSQLKETKKDLTLQFKLENSI
SEGQIVAKRADKKANTFEYNVEQVWPLKKGITKYTAQINKNEFLSGPINR
ALATWDFFLRLANMPDLPILAPNTIDFSSSGFFNVANNEFMAQLTRNDSN
NLACLTAVAPKIKQDITKIAVMGTCFSRNAFNSSPFFNPDYKAFFECSFT
QFHSSIISIMTEPANLINLDKYTDIKKSEKPFIEDDWKKDFFTNLKNSDA
DYFLIDLYPDVIRPVIWLNNNSAITLSYVIEQSQLLNDISYERILDHIDN
ETYFNEWKGYADQFIEKLTEIIPTDRVILNLGGFTTSYYDEDGEVATYKN
KMAIEKNNYFWERLNNYFLSKLPEAKVIDFSKKGYIGDFNYPFGHSFSHF
ESPYYKDFLKELIYITKS
ORF2884 (SEQ ID NO:238)
LAFIRKNSFLLFLILFLMSSILIHMFFYNFLTDIIVTVFLGLETIFIVII
SLEIMDVKKIRKRTKRIIEIYFLTLLGITVITSYLFY
ORF2885 (SEQ ID NO:239)
MIEFIIDVSINFITFAICFIPLLLSEKTKGILEIVGASILFAGIMIVGTG
IFISSSETLKSYIYVILVVQIIILCSELILVLWSKSKGKSTILSILSAIL
GIVALGIYIYYVIASFIY
ORF2886 (SEQ ID NO:240)
KQFEIAADYVYQSKEIIFIDIIGDETPAIGIIAENDYEGLEITLVDDEGR
LDGTCWIKKEDILALRFGGSVEQDLMNKLKK
ORF2887 (SEQ ID NO:241)
MLDKQQLYIVENKDIPNYEVDSGITGAEQNYIYNFKNPIPISEQELQDIC
DTFGEFMVSAYFLVDIFGPYEELPGDKKNGYKKVVLADESMEIKKKMFHL
FLEGDAYPDANKIFLFKEKKELNARKNMEIEDGMIVISPVLDVTAIGIFS
KGIDLAQFFK
ORF29 (SEQ ID NO:242)
VLQRRFWGIFCFAIFLFLFPTIGSAETSGDYEYTINGNEATITDYTGQST
DITIPTTLGTNNEYTVTAIGNGAFKSKRLTNVTIPNTVITIGDGAFTINS
LEQLVLPNSVQTIGRNSFSVNKLEKITYSTALKNIPSQAFLANNLKTVTT
PATVESIDASAFENNFITNITIQNPNLQMAYQAFAAQTVLSTLIVPSNHI
LPIENYIQFQDASAHLTTDNLFITDLANGITYNQAEKALNFSAEPLESTF
SLFTGTNRFDSYYDISEYGPSGKPFIYFKYTKPVLVSYKDASGNELATST
RLDGSIGENYVTTPKIIDGYTLKETPGNATGQFSETLQNVTYIYEKTAVQ
NGTVTVKYQDESGKTLAKDTVLTGEVNNTYQTKSKDIAGYKLQKVEGNES
GTFSTTPATVTYIYEKIANSDNTNTNGEMTDNTTLSTNDTVISSEATKKV
DKNTSNILPTTGDSKDALFFALGSLLTLLSTSFFFFKRS
ORF2925 (SEQ ID NO:243)
VSRIDIGEIQDFAFQLPAANQTGRKIIQGVKTTVTNYVEDGSLKGKAVEA
SKNYFQMTYIPLCDTIIEANNESEERLKRYIQDFHDQVDLSPNAKIDADG
LYELGQMIDRIESKKEALYQRMNSSTEGQMQTYRSQLATAYKQENILEKY
LAFEQSHGAFFDHLTDLVQGIQQTVRELQSNIQFDSQTGSYDLSKLNFAT
VNRMRKTLGKASATDTTVYNFASYSKVKQGVMWILSKDGKVDIKATEAYN
TASFNGELPKESNQATEEGELLKATLESLKQNKDPITGQEIIKAQSFGIL
TSLVFGYTTKGYQGKKLTISKNTLIKLRQADKTVIERIKKTSRIDYEEIV
KKGSKMPKHIIVENKQSLPGKANPSSSADLLNPDGSVKQRRYYDEKGRAK
EDIDFNHSDDGTHEFPHRHEWDWDRKPPRKPSK
ORF2927 (SEQ ID NO:244)
VDSNNEKLKQQLQTLQKQQKDAELSLYMLKHEQNERIWLEEDFERICYEE
RESLELMREVWQGDQARNFGYYLEDLQADEKNKWCQTFQAEEEKRQEKIN
TYQKNIYQLESKQQDIQKELFQ
ORF2969 (SEQ ID NO:245)
MNSNEISEIIIQQNKGKTIIKLLYTLPILLFGIFILFKVKESLLLGACGI
ILLVIVAPTYFTVAKHFFSGKPLLKVDQDGIEGWSKRFKWWEIEKVALRR
DWGAVYLTVYVRKNGGIHKYNINTKEIERSATELIKQIGYLKNKYE
ORF2970 (SEQ ID NO:246)
MIAITLFLSGCENGDTEKTDTEEQTKNVEDEGEKVEIESNEGKPQHEQLI
KIELPPKADYLNDETLEVYNQAKKKYDQTNQLITNDSITVLVGDYGYYDS
VWGSLDCSAVIKNGTNSSIKDLSFEVSVEDNAISGKTFLNSEALPLTKTQ
IGDFEPNTGVPIVIAFPEKNATGEGEDKKIDTKNVKIHISNIQYKVEK
ORF3102 (SEQ ID NO:247)
MSGKIKFNIAEAQNISLELKIAAGRYTQETEELLKVLKNNSLCDKDQDVV
EQRGRIEKNNQRLIEYEKFVNTNLAKSNSVIEELFMSVEVLYAQQVSEFR
NPNSAGYKELMGNVKAISYQKISQISGLGEMLTSKGSVGAIKDIRDTLLG
TLVDDTYLKLIDDTVEYQGENKNITDLYGDKISKVGSRRKKLDTPTKYLS
TAYLISDTLKSFNSYGNSKDSSRLAGDLTGIAITKGADFAVGKLATTALS
GFGVSGVKGAIAGAIISVAADKIIDPTVEYVKESKVEAKRDDWETKGIYK
GWKKIRDLKLEYTVGNYQAS
ORF3103 (SEQ ID NO:248)
MAKEIKVNPDFLKKVESNVTNYIDAQKEVSVELLAVRTNLASNFSGIACD
EIKNYITELMNDLEKEFGVFITKNHEKVKALGESYKELDGQLGQTFNYGM
ERTK
ORF3132 (SEQ ID NO:249)
MKKLQWLTNRLFATSILLITTLFIIPPTFAIADGSKVSFYEYIYGAPFRW
LTVISTTDKKGAFTEMFFSGNEGITIQWPNLMINFLLIFLAITIIFSLAK
KLYDKKNVKKDNP
ORF3135 (SEQ ID NO:250)
VKIDLEQIYTLQFTMGLRYRPMQIRHNIKLLLVALLFFSWIQGDFMSGLS
GDLSMFRVDMSLIVILGICFILLNPIKLAIRFQNSNICCIFFVFNG
ORF3136 (SEQ ID NO:251)
MYINLKSSDGNVITTKVGSCWTCVFLSFFGPLLRGDIKFFVLYVILDGAG
LFITLNYDRDIGIAIMAMVTLLFESNYNTWFIRGKMNNGWEPETEKDREI
LLEKGVIKTEV
ORF3137 (SEQ ID NO:252)
MKSRNWLKGLGVTMIVVTLILTGCGNGDAEKTDTKEQTKSVEEEGKEVKI
ESNEGKPQHEQLITVKLPPEAEYLNDETLEVYEQDKKKYDQTEQLITNNS
VTILLGDFCYYEPAWSSLTCSAILVNGTDTNIEDLSFKAEIMNKPMSGKI
FSDDKVPELTKAKTGKFQPNEGIPIILVFSEENPKNEENAEPQKINIKDI
KVKIKDIQYKAVK
ORF3138 (SEQ ID NO:253)
MVSPMPDKTVKMDKGSINPKLDKLISSLDNFDNINKKMGTIVGSQEISGE
AADALSESLLVLVSYGEQYSLVIRNLKEVIVDYCKSVEEIDKTAANAVSK
GV
ORF3140 (SEQ ID NO:254)
MIIIKNFASKNIRLIMALVIGVLVFAPVFHSKAADNNIGFDFKLKPNCAN
SGSSSRYRETSSVNNPWKVRLDNSTEGKGTIASFWLGTYNKNKNAVQGST
IMNVKQGAKTRYCGAYKVANKNTTYLAAENNNYNSKTYYVDGIWDEETW
ORF3141 (SEQ ID NO:255)
VLTNINYSFEPGKIYGVFGKNGSGKTMLLRSLAGLIIPTSGKIFIDNKEL
HHDISFPPSMGIIIENMELLPQFDARTNLKILSKIKNIASDEDIDNAIDR
VGLSPHIDKKVKKYSLGMKQRLNIAQAVFEKPAVILLDEPTNAIDDQGIE
LINQLLLEEKKRGATIIIASHHKEDIEPLCDMSIRNDQGKIIHD
ORF3142 (SEQ ID NO:256)
MTKTKFVIFIALTVITLLLFLVPKGIQYLKSQNPELLNTAESIKLQAGEY
TVGKDIKVGIYDMQVTKGSLSYYSTRLSKGDEIIGINLLDANKLYFEGSG
EVELTPAEFNPIKPSANIFTIQHSGSYEVGKQIPAGKYSLTYTIDKSSKK
KPFIQILPSYTDDARIEIQFETKPAYNINLKTGEILTVSKTISEELDTMT
VLLKKN
ORF3144 (SEQ ID NO:257)
LGNYVKISQDNKPIGWIDSENVIDEKTMDSIENDEESIIPEELIDKVNDY
VTIENNEFVLSNKAEDVLTSEEFVEVEGQIDRTNAEIEADETLSETHIEG
NIIVQEIYEDEPNQLLKASKKAYIKAKYTWWGMQIQFSHKAVVDFNDFYW
GAGTIGGLGANKRMGKFLAKKGIKIASRYAVCLSLFGGGLAWGMGKKDKG
KGVNLNCVLYVPATITTAK
ORF3172 (SEQ ID NO:258)
MSKERCACCNCLTIDVRGEFEICPICFWEDDAYFVFDKEEIYSHYQGSSS
IEDLLNIRSSANNSLTLLEARQNFNQFGACELAMKKYVREPTAEEL
ORF3173 (SEQ ID NO:259)
MEITLQQPTSADFPFIEWLWGDLATTEILGGPFSFPEETRNDWLKSKSQA
SNAYFIIKKGTESVGEVSFRDFEKGTAHLNIKVAACYRGQRIAQKALQLF
LDFFQTDCGGLVLLDEVRRKNEAGIKFLVKAGFEVIEEKEWTMVLKWSAQ
AEGGFE
ORF3174 (SEQ ID NO:260)
MNTLYYRVSKNTNFETAAREIFDLLIETQQQFENQPRFLKVEIDGHLNEL
NEFDNDMLKLQQEFGEIFLLQFFTTISFPLLTKKNPRKQINDIPNDLTIY
DLKYGNPAYKLQIENYYNTEFVLEKDVYVFLEKVSNLLKKYGKTDNYKIN
IENEEYDKLGLLIHWQSYMKELIVELFNSFVNGNLISNAANTRSLIEGYV
YLSIIKKERNPLLVQDWFLCNLIIGSKRYDDGTREILNNTLKELFDSNED
IQSRYKKGNTNNWLSTVIAKKNITFRDACEYLEEDYLYKDFQEASSFVHA
QDIQTKLSPFFSYSSIYGKLYIMIIYMFKTLLLFESSLVLKEEIADLELE
LIILGENYL
ORF3175 (SEQ ID NO:261)
MIYYKDKLKTIGTALTSLSFIFMFGLLTLLSIEKPELFYYGLITIFFIGY
LIVKLILPPKEWIQVTKTAFILHKRNETKTIHLANVKRISYNFQPSRPSF
GHPIPETMELFFHTENKLEKIECDFIGRTNFVALIDQFDAKLASLSEDIK
NHDYSYPRDLTFGIFIFSIVMLFLIFILLFGREFLFEQIGKVFV
ORF3183 (SEQ ID NO:262)
MKSQQAFMVEVTFYSNARKTVPEAGYRPHFVMELDENREYLGVEIYDIEV
DTLDSVGYAMCSFLYEQEGVGYSKIQPNKSFMVMEGASVVGRGKIVSFHL
ORF3184 (SEQ ID NO:263)
VISIQNNLEYNKLRWETLSGNVTGIWENNKFFLGSSSYPIMKYHYITANF
VNFEKHISENMPKISYHLSGYGVNFNEALVSFIGESAERYTYSLLPTIII
TRIIFRSYEEMTKEYKTDLICELKYINSYYSSEVCENYVTPNDTIQWIAM
NSLVHSDKKVWMPLQFVTMYTEEMFSNEKRYVTSAVSTGTACHETVEKSI
ENALIEYLQIDSFNLWWYGGFRARDIEIDITRNISSWFDNQVAVKKFLSK
FNVHFSDVSFDKSIYIVLCEIEAKNSSDAFPKYTVGVQGGYSLDKSIYRA
FMECLTVLEYNMNVTWTDKEKFLSVTQETRVIDNLDDNVIYYSKYGKPEL
QYNTNQLKNDTEKVTNLKALLEKLPTISQYAAFLPITPSEFRYMNCEISR
VILPELLSIHLPSYPPYYHVRYEEIGGVVNNIPHPIA
ORF3185 (SEQ ID NO:264)
MKNFDIRIGTQTFDNDTEIILKRGVIHKNELVINKEESSKEFVSTFKELI
KKKTIAISSEDAIYNDFETLTKFGFLTISKNQTLKPLLVVEDALFDDMKS
YFQEEIEILSSSEFLLKKDIRLLTENKDILQLTKLVDEKKEFLKNYNYIY
LITNIPNISLLRGFNKLMKETNCINTIAFFDNENVFVTCIEHGETGCYEC
LEQQILSHFDGVVTDYLVRSENNVSTAELMFVLSIIKKEIENTSIYGQSS
LLGNLLHFNFNNYEYTFNTNRIQSCCSTCATFNNILFEEQNIRSVNILKE
LMSSD
ORF3222 (SEQ ID NO:265)
MEKEQQTDDTWVLVEIVSLITNVERQRLRELSYTELEEFYERVVVEQ
ORF3224 (SEQ ID NO:266)
MKKIIALITAVALLSMIGFAFIPGTVANAANSSESSLTYKDVRSGFYFVG
YENVQLETGKTYKYTVAYEANVDMKMTDTITGQSAKAGLFTPKSSGAELN
TSYVSRTKNNVIDVADAGNKVFKHTFEFTAKENTKADIGVFLGAGSVLPT
TPETTSLWKNVTVTNETPLVQAEAPVISAEDKTINQNESFNPLNEVTASD
EKDGDITKDIHVTKNTVDTTKSGKYDVDYSVTNSSNLTTTKSIKVTVTPA
AISKNTAPVINAKDQTIKVGDVFNVLKGVTAKDNEDGDLTAKIKVTKDTV
NNSKKGVYQVTYTVTDSGNLSASLTIKVTVTQDGKLIVNPSDPTKPSITK
TPVEPTKIEKDPENRATSVLKASKIPKTGDSSMIWLVFVGLGLTAIGISS
YQKKANK
ORF3277 (SEQ ID NO:267)
MWLFFKRDILWFLQGNKWKIIGLGMLLILAILINVVNAKNASGTIADVFL
SFLKQDNGAENPLTSSLNWIIIQSLPVFLFGSYFYKELFALEEFITIRFN
NRMLPFLSKILLIITLMLIYYFVIIGLVVFISFLFGIRFDVQPTLLFIDL
NMPLYEMGLHFFVGGIALIMLQLLLSIIIKPFYAITVVLIIIVTNCFITN
FWIIGSVSNVAGFAEANNWLLLSIQLVYIILVMLIGGNIYRKTDLYKLN
ORF3278 (SEQ ID NO:268)
MKNRNLVRLNFDFERRKYLMLLLIFVFIIVSRLLYTRNVETDALYIVQSS
VSVEVLFIILSPFCLWMNQILCFQRRELAVVRIKNKYTLWKVNATVILWN
AFLLAVLTNALNYANGVIVMNSQIVQIYIYSFILFGLGLVLVGVLQNILL
VITSNKAIAFFVVFLVFFFDTSTIKLQLISNLFIVNPNDLTDLLSFAGRV
FCLVGGIIVLFLISWLLTEKKDMFRTSKKKVR
ORF3279 (SEQ ID NO:269)
MSRFLKIEFNRVFKSKSFLAALALGVLIVLIQQITVARYYSTAEENVFLY
LTGYDTTGLGTNLYYLLLPCLVALAGADLLGEDRRSGLDIFSRIRGNDKQ
YYFSKSIVAFIAGGVVFCLPLIMELCALMLVYPSTPLDYFVAEVPVTYGA
NFSNIFYNSPLTYELIFLVIGFAYGGLFALIGILVSFFSSSKYVVLLSPL
AIYYGVWIVFSLIGYPEFSPFGFLTPKQGYPLNFYIIWVEFLLLLVVIIM
GIIWRVKNEKS
ORF3283 (SEQ ID NO:270)
MYKDGYTSRNNTYYGEFYGHYAGGKDKFSYKLEKKNNKNVKSQLIVDLFV
I
ORF3291 (SEQ ID NO:271)
MLTKEILPIGSVVYLKESLKKVMITSRLITIQGDEEKEFYDYGGVVYPEG
TKDDNILAFNAEDITDVKFRGFVDDDEVVLVKRMREWQKEEFGVSPEEED
EILEL
ORF3316 (SEQ ID NO:272)
MRYEAKIISENPNIEEELKIKIKDIELLCFVEEYKCSVEVGQEYIVELET
VVFDDLDIEKSTLQAKEITQVADSFAYFIRGVFHPSSKKIDSGIEIDLAD
EDISDFWYLENQFVALNVDRFNIDVIEKVNK
ORF3324 (SEQ ID NO:273)
MARIEYEEKIQKSLLVLYFRGSTIQSICREYGIPRYEFHKWMKLHDADKL
ETKEVKTFLQIRELKQQKNKLEEEILFLNEAINLLESP
ORF3325 (SEQ ID NO:274)
LKRKLVLAMVLISFCGMFLLSPERVQASQTVVEVFISEDDLDVSEENIQE
IPKKELAASSDKDNKQPILPKAGDTVTNYTLLGLVLVLIWLIMQRRSKKK
E
ORF3326 (SEQ ID NO:275)
MNKLVLSTLSVAAMGMVFFSGGTAYAADKEGNTVVEYSVEGDYTLVVPEK
VNLSNDNATEMSVKTINRNLEPGKEVEVTLSSGLSADGEIELQRVGAISD
VITSSFKSNNSVVPMANPVIGSFSGYANEETEVSKIQIGNPQGDKKAGAY
QTTLTFTAAFK
ORF3327 (SEQ ID NO:276)
MKKFSMRVVLIISVLFIAFGSANVSIAQEMDTTNKLPEEELGSLDTSNLI
AEEVAQDKPAEVDNLEEIPTTDELNQNPEVLEQPVVDSDDPDLTVVSSGD
FWTLYYNSANDEYSMRNFGNVPSSKPTAWNSYLKYIKHIEIEEATLTGSF
ASYFDNSAFPALESVRIEQCNLSGVTSFRTAFNNHLTLEKVIIKDNDYPT
TSSLLTTEYMFSHAIKLTELDVSGLDTSAVTNMKNNFGGCNSLEELDLSN
FDTSSVTNNSGMFGYCESLEKLNVSHLDTSSVTDMNAMFYGCTSLEALDV
SNFDTSSVTDMRANFADNEKLEKLDLSTFDTSSVTNMGTMFKDCTALKSL
YLDNFTDAAIMTDMFKGTTSLTYLFVSENLSTFTSLENTSWYDEKNWVQF
SNLSQLQTYHRKQSEPTGYRKGAFLSLTMDAMGGEFEDAEEQKVQSKISG
EYWEEVIPVKEGHYFDGWYLDQNFTNKFDFSLPATVSATLYAKWVENYTV
VIPASISLNEATELKVEGINRGDKNLSVGLNRTATSVSESNKLTLANTAD
TTIQCLAPLSWDGSENNPKNAILTLAPGSEITEGEAVLAIEAPENIQAGT
YTGNLVFSINYE
ORF3361 (SEQ ID NO:277)
MKELRLFLDYKCYPVWVYNDAGILKENDLPDELKQDEYDKLFIDTEIEFR
YEAFKDEKEKEEFFHEFIEVQKHLKETLGKEYNIVNKILV
ORF3362 (SEQ ID NO:278)
MKKIMKKDDYSKMPWVSAEDLYLLFEQALKDFKQSKLSKKEFFDILDELT
MRQVDTYEILKEPLRGQLDNELYNLWNTENYDDVDIITSLLINLGLKNTY
NKMKKSIEDTSEISPEILEEIQDAIEEVGDNIDDPYQDYMKKM
ORF3365 (SEQ ID NO:279)
LFNEKVKEMIRELDKYKLQDKWVRFEKLSKNKKIDSETFQDEFIIITNGI
LHVENKKFQILHFFSNGDIINQQVAKISGENELRLVCDTDVSLIFIDREY
FLNYATNKPSYMEWLLEETLINNKNLYNELIKYDLSAEERIVYALQYLCD
KLEIESENGYQQIPKYINKIKMAKYGKISRKQLNEKIILLLDKEVLKEKK
GRFYIKKSA
ORF3378 (SEQ ID NO:280)
VLNKKRVIVGVLLLLLSLISISYYSETFKLTLSWLLFAVVLTVLYFRQEK
NFQFQWSTLITSLIISLFWMASSFNGGPYGGNFVFNSIILAGTFLVITIF
VLLLLLEMRTEYKARPNRKVKWPFFALFTSIPFVVWMISFLAYYPAKMTF
DSYYQWGMAHGIRQYSQWHPLLHTLWIETTSAIYDSPSSYIFSQIIVVSL
IVGFAIYTLVKMGAHIWIGVCISIGYAIYPAAMFYSATAWKDFPFAAFIL
LFTVLILKIVQSNGMWLKNWWHLIAFVLVAFVCINLRNNGMMIIIVSLLC
LLIFMKNFRLIITGILVGTLGLNFLFGLVMTNGLNAQPNPLNQALAIPSQ
QIGATFYNDGNFTPELKEYFTSILPEENWKKDYNPYTVDPIKHDTKYNSS
VIEDDFGLYIKNWFKLLTANFGTYVGAYLDQTAVIWQFYSPENYKVFFDT
SANIQDTRYDVRAFAKFFPEGLSEEEINKLGYEVYQNEYKNATGKDAVSY
NEYKRRIDDSTNPLISISKAPSLKKITDSIYAKTTNEWQNYLLKGAIPLV
LLIIAIAAVCLQRPKKKLLIFAPVVMALITIAIAMPATDFRYSYSFIFTV
PIVFFATKLKNYKENQF
ORF3379 (SEQ ID NO:281)
MGILNEKVAVLLPCYNEELTIGKVIDDFKKELPNADIYVYDNNSKDKTFE
IAKDHGAIVRKEMRQGKGNVVRSMFADIDADYYLMVDGDDTYPAEYCHEI
LEVLRNKEANNVIGDRLSNGTYTEENKRNFHDFGNSLVRNTINRIFKSNL
RDIMTGYRGFDRYFVKTMPVLSPGFEIETEMSIHALENRFLVKEIEIDYR
DRPEGSESKLNTFSDGFKVIMTIVRLFKNSRPFLFFNLLASLFVLVGVLV
GLPVIIQFAQIGLVLKFPSALLATGLIIMGMLFFICGLILDTIAHRSRQS
YFLELVKYRERNPLN
ORF3386 (SEQ ID NO:282)
MKKLLLSISIIVGLSLLASCGLEKEYKPDIKVNEKNVSLKVWVDLNQGDF
YRKVVEDFKKEHPDKDYDITVIESESGRAQEYVQKDPEAAADVFITPNDR
LGQLVESGAVYQLTKYTDDIKKNNTPTSIQAATYQDKMYGFPVTAEAMFM
YYDKRVFSEDDIKTFSGITSKGKLGINIAEAGADYRETPWFIANGTYLYG
ENGEDPYGTTFNTPEGVQVLNWIGELKNNPNIVAVNADEISALRSGKINA
VFSGVWNKDAIREVLGENMGVAVYPKADFGSGQVDMMAFQGSGIYCVNAF
TKSPLDAMELADYITNADVQEKAFKELGKIPSNLEARTSSTVEKDDVAKA
VIDMTSGKHSVLMPKIPEMNVFWQHMNPLLVDTYKGKIKKEDYPEALDKL
VKDITPAK
ORF3401 (SEQ ID NO:283)
VKNRLAYIFNAFFILIFGYLLCISIFKPLEISFNHPSIFILFSAAALLVL
IGFYQFSTRLNTKGDGVITIFLVSLIILTQIYLLFSLQMNSYADAFLIKG
EALNMLSNGGHATTQNYFLMYPNNIFITIIRYWLYSVGGTLGITNTYLLE
SAFLFVCMNITIFVLYWIVRKENGNKFGNIYLLIVLFCVPLFGYIWYFYT
DTLVLPFTALIALFYYLYTKSSKWWYFIIIGLLFAVGYQIKPNIIILLPA
MLIHLCFIRNWRKILLNTVIVAICFFGLSTVFTPIAESYDFKKDPTIEFP
QTHWIMMGLGDPAGRYNSNDVAYTSQFKTKEEKEEANIEKIKERIEEHGP
LGLIKLFDNKVLNTWTDGTPAYTWYVNAALDYPAPYDYFFGDKRVVTELP
AQLFHIINLFLICLGALRFYKKREFDMSFFVNISLVGVWLFHLFWEANQR
YIMFITPLMILSSIYGFKFIVESLYTKKFDLKKGLRKGFLIASFCVFLLS
TVAFAFIGNSVAGESQDINKYLVKQSYAHIDLPVTSKQIVKQTFNVDSPF
NSIQIAVLKEPDEASKYRLKVVDKTNKKDIYDEVIAGSDFVEATNYQINV
NEKPKGKTEYVIEVYQVENKNPEKPLVLGTYTPDAVDLYPYGALYVNGVK
KEKQDMGFTVSHVASEPIIPKYVSAIFDLGVIIIFAGTYYVFRRKTGDNR
ORF3402 (SEQ ID NO:284)
MINKKWMKIVMIPMLVVPMYGLTTVGGQLQDSLTGENSFVKEVEAATTAS
QQAFIDKIAPAAQASQEKYHLLSSITLAQAILESGWGKSGLATQGYNLFG
IKGKYNGQSVIMTTSEYVNGEWIKIDAEFRKYPSWNESVTDHTLLLVNGT
SWNKDLYKKVVDATDYKVAAMELQKAGYATSPTYGASLIQVIENYDLAKY
DVLYDKILTQKSTSGKATVTSPTGNGVWTLPYKVKGVQSVSPASTYANKD
IDLVSVATTKRGTYYQFKYNGKVVGWVDGKALTIYDSVNYDKVNVGRAKI
TSPVSNGIWSKPYNVYGREFVTNATTYAQQEIKLLREAQTAKGTYYQFSI
NNKTIGWIDKRALTIYFYDSIISSKNVNLDGQITNPTGNGIWTKAYKLEG
TTSVAQATKYANKDVKISQQIETQHGTYYNISIDGKAIGWLDRNAITLYD
QEEYNKTVAIDAVVKNVKGNAVWTEPYRTVGTKLIGPAETYLNKEVEVVR
EAKTPKGTYYQFKSGGKVIGWLDKKAFDVYDNINYNKAVNLDAVVENVTG
NAVWTAPYKSKGVKLVTSAATYKGKATKITREAQTSRGTYYEFSVDGKVI
GWLDKKAFDVYDNINYNKAVNLDAVVENVTGNAVWTAPYKSKGVKLVTSA
ATYKDKATKITREAQTSRGTYYEFSVNGKVIGWLDKKAFDVYDSIEYNKA
INNTGLLSNAPGNGIWTEPYRVIGTKNVGQATAYANKTVQLIREAKTTHA
TYYQMSVNGKIVGWVDKRAFTNVK
ORF3410 (SEQ ID NO:285)
MNKKRIIILAVLVVLLIGGVVIGVYASGNSAKDNNESKTTAKKATSTPKK
AIDTEKKETTTKEATTKDSVTDDKGVVTKGSSDVEKNAPAKNNSSATDKS
NSPTTPAFSLSSTGFKTSNVSSVLGGTVTTTYLSSAPSFEKIFENLTIEV
NQYKVEHVVGANKAVSASNPESYLANKNGYVITLDISIKNTSAKDKMYKA
DQITLIGANEFVGGSLDNFVPSNFHLIGSKADPNIFTAGKTARGLLTFTM
TEAVYNDLAADSKIGVPNPDKFDASVSEANAGDDVVASFPVK
ORF3457 (SEQ ID NO:286)
VSTTKNNIDYIKTVQNIKSFFDEFQYLVFLLGSKNKIKLNTDGLIEIKVL
TGNKISLTPIGHLVQFYLGILNDMKALHRFILIKCYIEINKKIF
ORF3501 (SEQ ID NO:287)
MPDWDFNNPESMKAWDLASGSYAEQVSGEVRAVVGSDLRKGNIWENVELP
RLKNNPNVTKITTIDPKTGLEKIIFERK
ORF3593 (SEQ ID NO:288)
MKLIMNADDFGFTRAINYGIFDARNLGVLTSTTLMVTMPAFEHAVDLSKQ
TPTLGIGLHLNLTLGKPLTNGASLVNQAGELIKPKFITPEYPYNEEEVYQ
EFKAQYHRFVTFMKKKPSHLDSHLFSTDIYPVAASAAKRLAEEVEIPLRN
HDTVGFQHVEFIWEKPLEIPYGAYENLDYIYDYAESILCYDYVEIMTHPG
YLDTFILENSTFSTPRANELESLISPRMRQFLNENNVELISYHDIPKK
ORF3594 (SEQ ID NO:289)
MEGTELQSFKIISSAGDASSSFLKAIRHAEKSEFAEAEACIEQANQSLRE
AHHVQTSLIQEEAGGDSKEVSLLLIHAQDHLMNAFVYCDLVKSVLNLYKR
LDEK
ORF3745 (SEQ ID NO:290)
MKAKKRKEQTFHEYFKEWVDLYKVGAIRSITLQKYYVTEQKIQELVPELK
IKDLDRYTYQQLLNNYALTHEKQTTMDFHHHLKGAILDAVDEGVLNQNPT
RKIVIKGKNPRPKKAKFLNQFEVQVLLKELNLKEDINWDWFILLIIKTGL
RFSEALALTPSDFDFSTQKISINKTWDYKMVTGSFQPTKNESSNRKIQID
WQLAMQFSQLIKMKDSDKPIFVKSRVFNSTINNRLKVLCQNANIPTITIH
SLRHTHASLLLFAGVSIASVANRLGHSSMTTTQETYLHIIQELENQDNDK
IIRHLSMLM
ORF3792 (SEQ ID NO:291)
METINHFFEWAKNNNWQVDLSAVEKNLPEQILKRYGKLPDAYKAFYRQLN
LCSNAGDTCWFLSEEDFLENEVDAFSWNSFEQMSLEAAEGDTNLENEVKL
FWNAHLPIMMSVGGCYEYYAITLNDGNIVHGSEPEFEESSIVAESFADFL
LKIVAGEIVIS
ORF3793 (SEQ ID NO:292)
MKVIHFYSENADYGCFSNFSLHPIVIDGVTYPTTEHYFQAQKFIDKKIIK
KVINTKKPIDAAKLGRNRDFPLRKGWESMKDEVMLKAIRAKVAQHSEVKE
MLLSTENAILVEHTENDHYWGDGGDGSGKNRLGKILMKVRDE
ORF3840 (SEQ ID NO:293)
MVVKQMDDWSTLIGELEHNTPEKVWIVREELNISEVFEIQKLEAHFILVL
EGVLRMENEHQQILHYFRENNVIYQSPYELRVQNKLRLVAETPAHIVLLH
REFFLNYATNQPAYSEKLVRAIMDNAASFMFELMKNDLKSEDRLAYSLQQ
LCQSLELEVKNEFYLLPHYINKNKLALYSDISRKSLYKYLQNLERKGQIK
MNGNQILVRISRFTNSENVDWL
ORF3913 (SEQ ID NO:294)
MLKKTKKNLNEIKTIIQQKPFDYEKAENLISELNLEEALLELENILLASI
NNSDENARIFAYEYLYYFDSEAVFQAALIGTTDDDDLVQMCSIEILGNLV
KVESLPYLKKALGDNNPDVRCFAAESIGFVGTDEAKAILQEQLNRETDSF
AKVGIYYALYLLGREEMLPKLLSLLDDNYHLTVIRSLDVLRDVVNQTNKE
NILLNIEKLLKRDIPISVKEKAEVVLQEIKGS
ORF3914 (SEQ ID NO:295)
MINLEWKELDQLEIEEKVQEVLDYSYNTWMSDKKNIRYFVRAFYIRWDML
VDMYEVEDDETEGDKLKYMYDFGISELGNITEVDWIMGYCMLINPIYFEE
NDNYLELEEKGQEMLRNVAINNPDDVFLTSFGIPEKDYLKWKIANREQLI
QYGEDNFSYDSEFSRYFKHIINCRANEEVEKESFLKKIVRRWKQR
ORF3921 (SEQ ID NO:296)
VLPLFYVISHYSNLPFRAWEQRKLGDLVVDYVEKTSVQNQFPMLTSSQQK
GIVLQEDYFANRQVTTENNIGYFVLPRGYFTFRSRSDNDVFVFNPNDIID
RGIISYFYPVFTLKSADSDFFLRRINNGIQRQLSIQAEGTGQHVLSLKKF
KNIVANFPSEGEQKKIGSFFKQLDDTIALHQRKLDTLKQMKKGLLQQMFP
KSEEDVPKIRFADFDEEWYQRKLGEEFEKINERNDGSFGKTHWISVAKMY
FVEPNKVLSNNIDTRTYVMRKGDIAFEGHSNTDFKFGRFVANDIGPGIVS
ELFPVYRHKTNYDNNYWKNAIQLEHIMAPIYSKSITSSGNSSNKLDSKHF
LNQKIYIADFEEQEKIGSIFKQLDNTIILYQNKLNKFDILKKAYLQTMFI
ORF3970 (SEQ ID NO:297)
IIALYSTREGFEKRYGFVDVDKDNSYKRLKKKSFYWYKKVIETNGNDLSY
ORF3981 (SEQ ID NO:298)
MKKVSSLLTQDQFWGIIDNSDKGSKLEELLEKLSEDELFGYDYWWNYFHK
KSYNQSLWAVAYVVLGGCSDDGFDYFRYWLITRGKAVFTSAVENADTLCG
EFDLLTEDEYPENEEVAYLVMDIFENKLGKDFDDAENEAESRIEFEEVSM
PPIDFEWDEDDEDSIKKVCPNTFAKWWNNDKF
ORF4017 (SEQ ID NO:299)
MKLVQKVYYLLFRLVGFLPRKKDLVIFESFSGKQYSCNPRAIYEYMEEHN
PEYELLWSVNPKFVAVFEAYGVPYVKRFSISWLFKMGLAKYWISNSRLPL
ELPKPKKTIYVQTWHGTPLKKLGVDIDEVHIPGQTTEQYKADFVKEAQKW
DYLISPNAYSSAIFRRAFGFTGEMIESGYPRNDILFSTDKELKIANIKKE
LNIPEEKKVVLYAPTWRDNDFYEAGRYKFDLKIDIAKMQEKLGNEIVLLV
RMHYLVAEHFDFTQYGDFVRDASNHEDIRDLYLVSDLLITDYSSVFFDYA
NLQRPMLFYTYDLAEYRDTLRGFYFDFEKNAPGPLVETNEELMSELEKML
ENPPKIEDSFLEQFCTWEDGHAAEKTVKIVFAEK
ORF4018 (SEQ ID NO:300)
VGILKEVAIYIYMLAVKITGCLARIFPVKQKVVLLVSFPENSTAIIKQMN
EMKVTPKTVVFYDPRVDVTGFNFDFIQLKPKKIKHFISLMFHLNTAKVVI
TDNYFVELAGLKERKNVTCIQIWHANGALKKFGWEDKAAQKRSARDKKRF
QEVYRCFSKVLVGSDEMAAIFQRSFLLEDSHMLKLGIPRTDNFFNQQQLK
ENAERTNTKLHLSNKKKLLYAPTFRDEELHSTTLHLDIAKMKQALGNEYQ
LILKLHPSISNDLDEVVDDFVVYADKETPIETILPAVDILITDYSSIPFE
FALLEKPMIFFTYDLEEYDKARGLSDGFLATIPGPFVHTTEELIQLIEQE
AFDLEMVPAFAAKWNKYSDGHSSERFVSFLKEQLEK
ORF4100 (SEQ ID NO:301)
MSYLWTSIKMQFRIPVSVFFSLLFPLIMMFANVTSYGNFDIGEGYHFVDK
YFLISTGMGMLPIALISFPIWLGESVQNKSYKRLEYFGLSAQKIIVSDVC
SYILLTALSIFVNILFGYLVYGLHIPDWQYFIAYVLQCLYCNLVLLIFGA
LLALIIKNPRILMPVGMCLLFMFYIFTGTFSSFSELPKSFQAVGNFLPMK
YIMNDLFNVWTQNKLFISKFLALNTIYGIILSCALIAFLLRSRKIKIKS
ORF4101 (SEQ ID NO:302)
VHNNNILETKNLKVTINNKVILYLDKEVCISEKDKVAILGDNGAGKTTLV
NSILGEKNSSGEITKKFKKNDCGVVFQENAYNDLMKVYELITLVLPHLKK
KEPAQFLHKYELESLRKKYIKDLSGGEKQRLTLCLVLESHKKLYIFDELT
SGLDYKKRLGLLALMKEKTKDATVINITHYFEEVENWATKVLILQKGILL
FWGTISEFFSNFPHYSVIKVDQIELTKIDETDMTFMQSTDTGDGIAVICS
DLQIQEETKKILDKKNVTYNTIKQNIYTTYLVAYLRGTSSSEQEVLI
ORF4132 (SEQ ID NO:303)
MKRKISSIIVVGIMFFQSLTTYPFITEAKENEQKEEINKPSKITKGLTNS
LKYTKTILETGDTYDSVFPDSALAKVVAKEATGSENTTQLVTQADLNKIK
SLNGYNKGISVLTGIDLLVNVTSISLNNNQVTDISPIDQLPNLVSLSVKN
NQISSLILNAQNQLPKLTTIDIENNPDLNTIDIQDQPQLVDVKTSGYTGL
RKLTTVIAKNNPELVNLGQYTIRNVYFSQVASLTKVELVNLPKVRKVNLE
RNSINELKVTDLAIEDLPLGENELTDTVFDNIQNLPNLKTLDLSKNQLEE
VVLDKTDVENLPNLMTLNIQQNLAIKLINVQDQPQLVDVKTSDYKELSAL
TTVIAKNNPELVNLGYPIMQNVYFSQVASLTKVELVNLPKVRKVNLERNS
INELKVTDLAIEDLPLGENELTDTVFDNIQNLPNLKTLDLSKWQLEEVVL
DKTDVENLPNLMTLNIQQNLAIKLINVQDQPQLVDVKTSDYKELSALTTV
IAKNNPELVNLGYPIMQNVYFSQVASLTKVELANLPKVRAVRLERNSINQ
IELNNLVSVKDVNLNTNKITNDSIEKFKGMPILATLNLNKNQITNINMLD
DFPEMTTLNIDLNSVSVLPSNLKTKMPKLSRISALNQTVTLDKAIVVDDS
DLIINNEISNFGKLTDPSPISNFGTYANEKITWSSERIKNLTEVSFKFSE
LINVTGIDGTFSGKVTQPFKKSTTPVINADSEIHYPQGTKKTEAEFLKDI
QAQTTDDLSIKSDFEIMVNLKKVGKYTVILNVENMDGIKANPKEVTVYID
AVQGANITVKYEDKSGNKLAENSILTGNVGEEYSSSEKEILGYTLTEIPT
NAQGEFSLEEQTVTYIYSKNPVPAKDITVQYTDEDGIEIAPTETLSGNVD
ENYVTTAKTFTGYELIETPSNAEGKFSENAQTVTYVYRAIKADPILAKEV
TVNYQDELGAKISETEVLTGEIGETYTTVAKTIDGYTLIKSPINASGIFN
ENPQTVTYVYQLQNNPITANITVKHLDENNNELAPSEVLSGIVDEAYTTN
PKEIKDYSLVKVPTNASGKFTTEAQTVIYHYKKNSIQTSSYITVKYVDET
GKELAISEVLNGNINDSYATTAKEIKGYTLVEKPANATGKFTDQDQTIKY
VYRANTDEVNLDPDVPARNPNGDIQPMNGAQPPIEVPKSLPKTGSQPANL
IFGLGVLLVFLSTQWLHRDKRKKDKFHNC
ORF4133 (SEQ ID NO:304)
MENYVSIVKIENNLSVCFYNSSEKVVAIAKKMNEINEEAYMHGYNWEAFF
NYYLPKYAPDVLEGMGSDPEAGMYVAYYTLSPETEARAEKLVQVITNLIE
NEELLYQIIENEGNNISWDN
ORF4135 (SEQ ID NO:305)
MTFLNTLKLNLENEKKRMLSDAFMKKQEGIIVNYIVTCSKDSAIGISKKA
IDILLIINENTFPEWPNVDRWLSILPKYFTDSFSKSKILHSEDWLFEEWL
YWFEPENRFWFLGELDPVDNEHLKISIVVQEHPFPVESLEVLLMKLGTSE
LHEIGME
ORF4188 (SEQ ID NO:306)
MNKVTKIGDVAEKTGYSITTISRAINGNPNVSDKTKKKIFAANKELNYYP
NNIAQQFRGQGTKMIGVVISFITNPFFAYLVDAIERYLSHRGYQVVMLQT
LENPAKELQFIEMLQKKQLDGLIMANLENDTEEIKSLVESGKIVLCNRYL
GNENLTIINIDETKAAYQATNYLIKCGYKRLAYCTGGIKNKNDYRFKGFM
QAVTENGLSFDESLYFEKLLTIKDGEELLVNILEEKSTLPDAIFSNGDTV
AAGILYAAKKYGIAVPEELGIIGFDNQPIAEVLNPALTTIEQPIKELGEY
SAQVLLANLQGTSVPVAPDLETKLIIRETTK
ORF4327 (SEQ ID NO:307)
LNYDERVRVLIELKVDLSGKLEMMENEEELLCRQKHDFVSTWSNAKTEDA
YRKLNEAVRKKIKETTEYAREIDEKITARIKRIEAAYKAEYQSNRSYTWR
IAEIDPIKFKQKYNERLNQLSYLSCDGSVKTRLIKEFRQNNFLK
ORF4369 (SEQ ID NO:308)
MKKTRVTWAIVIAIAGLLVSVISLSVNITQNRYNVRGVDSSSQWKTGFQK
NQTVCAIETKKHWTKGSVKYGVNQTSGTVKTKYSIYSKKK
ORF4457 (SEQ ID NO:309)
MRADLSIMWVEDTTSWYTEQKDLLNMDIESLGIDIEFTRISTIDDFYKLV
RNDNTGFSKYDIFFVDYALSSDTEQKGSNIIVKLKDMGLTTDILFYSSKN
IREIREIVKKDIEQFEGIYLADRDKDFRDKSFQLVEKNIKSLSSIKNIRG
LLMDQTSENDFIVKSYISKEINELLPEQKEKIYKWFEKEVDKNIEEVTKT
IKGYVERKPSTIEKYLKLPSYILPVKLQYNLLEEIININGVETFVDSTID
YYLKKVVSNRNKLAHRKLVMCRQQQHLLQYNNINTFLNDDSNVCDNHEGD
VVSIDDWIQLRVEVKEYGKLFDDLWEKIKS
ORF4507 (SEQ ID NO:310)
MIDYEKKGFFNIHTLVNKDNANISNSDNKHIYSQLMSGNGNSPMLGYLLN
NNKQNLNDFKSIMFYNESNLASLINEAREMEELIDSSTLFLSKTNKKIKT
HFSKVLEQRHSTRNFVYETMDLSTFSNIIQFSFGLSTRKLVYNDLQSTTR
HYSSGGGLYPIDVFLYINNISGIAKGIYKYQPYTHSLHPLDVDKIDVESF
FVGDNIDTSNNNFCVFFGYSINKNYVKYGELSLLNTFVELGGISHNFDLV
CHSVHYTSCPIAGFNKSYLEKLLYLDGINDHIIFSNICGKE
ORF4563 (SEQ ID NO:311)
MKKKFSNPTFRIIASIVLGILIGVLICFVAIGLGYIHMNDGTLKEYSVKI
FGLTIFDIKRVGSEMVGTPNNTSMMFIGVIISMIIAIVVEIIVSLKNRHR
KETAK
ORF4578 (SEQ ID NO:312)
MKKKLVVLGLAGAIFLSGMGILNVSAANYSDTKFSFTLGKLGANDYTGSR
QKQNTTSSYVKLNSIGKGTMDTWLLKSNGASVRSKYVTVRQGESKKIANY
AYEDYGKCNVKLAAETSKTQFVRVTATGLWSPDSI
ORF484 (SEQ ID NO:313)
MKALERFLSKWLLPFAKVLESNTQMKAVRQGMMALVPITLVGAVPVLFQQ
LGGIPKLPSWIAAVSNYINNITSPIYFATFGLMSVYVAVFVAYYYAKERN
LWDIGAIVTALMSFVVVAVRPLESGGSDVAYLAGEGIFVALVISLLSVEI
LHIFKNKLKFTINLGQGVPTPILRSFENLWPILFSVLIIAILSFGIETLS
GIRVVELIQTLFSPLTSLVNTLPGIMLIIFIQQLLWWFGIHGYSVMAPVW
LSVAFQNVDANAAAIAKGEPLSSMLIFTPDFMWSIVGVTGAGVTGALVVI
MMFSKSKRYKTLGRLALIPTFFSINEPVMFGVPIVLNPRFFIPMMLAPQI
AALIGWFSIKLGLMNPFTMVSPYVPVPIGAIVASFDWRYVIVLGLILVCS
ALIYYPFFKIAEKEAILQETSGDQEASLDDFDF
ORF485 (SEQ ID NO:314)
MKIMLVCFGGLSTSILVKKMEEAIAASEKFKDKGITIEAWGKDEFSDHLD
NVSIVLLGPQLSMAYEQVIEATKEHGLNVPVEVIDKEDYGNMNAVPILIA
AFKKIKEAGTNTFKTEGN
ORF577 (SEQ ID NO:315)
MKIHAKAKKILVSLIAIMLFLSLIPGYAPMAEETSTGVEVPEKKAGEQAP
TEVKEERTENEVVFNNHDGSFTKQIFADSINMEVDGEMKRIDANVEKEAD
SDMIVPKQTPLELGFLDKMEDGAYQKLTKAGAEVTFRLKGARTGENEQAV
TDQPATYKENEVTYENVFPKTDLRHLTFPQSVKEDIVLHEPNQVDTYVYQ
IETKLGLELAKNGDVLFKNKSGETMYTLPKPVMTDSNVGAETGFAALSEN
VSFEVKQLTKTVYELQLKVDTAWLNDVAREYPVYIDPSVRLDEVYNANIN
SAKPTETNIGSKLWDSGQNAYTLKLGKWDNSTGNNAAYLKMDTSTLNKAT
ISKATLKVYNIWHMSPTVKNDLWYYESNANWSPWQVTWNTAPATTRIGSV
NVGRGEWANLDVTKTVQAWASGVRPNNGFRLATNIDQNYWKKLVASENNK
NYPYLEVNYTYAQPEKPTVKTNSNGVGTGTGYMDLSWKAVPGATSYNIVI
SNGYNYEYINTKSTATTWSTKGKKIFPTNDEIANGEFEFHHDGKGTEFAL
DPRAQYENAFQAGSTFGLRNLTRYLFRVQAVFPGGESPNSDLVFAYMPIE
KPQPPAAKAYSNLAHKETGYVELNWEKSPMADGYKVLVFNGKAYEEYDVG
AETKWTTQNKGIWPTKEEIAEGKFALHHDGKGDELAKDPSPVYTNSGGNY
KERTNYWFRIVAYQKAGNNATSIQSEPATPSLPEVVNKQLGMVDYWTSVP
VRGGEVNATNGNFLFHETDFNLEGNGPSINVDRTFNSQDDATGIFGKGWT
STLEEKLIEEENGNIVWVESDKKVHRFTKKGDKYVAPPGIYSEITKNADG
YLKIEEDKSETRFLADGRLKSEKDTKGNELTYEYTDGKLTNMRDASGRTV
TLAYEGELVKELVGPEDRKISYTYNDKQELISSSTARGKIYCYGYTDGLL
TSIYDPKHTEEKPYETTFAYEEEKLTEITDPVGKKTTLSYDMEEQQTTLT
NEKKKKTIYSYNDAGNPKKEIVDADGLKLTTTYTYESNNLVKEVNPKGQE
ETYSYDADGNITQATDAYGTESYTYNGNNDVTSATDTEGRKTTVTYDGAD
AVSETLATESQVSSVTQYDAYGNPIRGSGELSSAGNLLQNSGFEKGAGVS
NWTLIQSDAKGSMTFDSTQSAPGTLGGSGSVKLTSEANSTVKGYSSVTQR
VDVEPETTYTFSAWIKTSGMTNADAILIGRLQDANAKDITDAGVWQSNRA
TSIKKNGGWVKRQLTFKTSKNTRQVLLYLDNEQPAPHKGKGTIWYDNVQF
EKGSVASSYNPVVNSSFEEHNGTLPTGWARSGNTALTQAKVVDNESHSGD
SAVYFERKATSEAYTHIVQDVPVNQKEAKALTISAISKSEDAKANGSVAT
MSNDYSIWGTIYYQDGTTSSVQGQFPLGTNDWNRSAVVVKPTKPVKMIKV
YTMFRNGLTGKAWFDDVRVIEGEVLTKNEYDASGNYVTASYDEEGRKISF
TYDIYGNTTSETDEKGNKKTLTYDADNALIDTKLANGTSVAYKYDDNGNT
TEKNVTASGKTQKNIYEYDVDNKITAFTDALNRTIKYEYDAAGNETKAIM
PNGRVTESTYDSADRLDGIKWNDKLAFKFQYDPNGNQTKVTDEINSIVTD
KTYDDANRITKVAERGGDVSYTYKDKPTKDNKGKTDKVGEVAINHGDYTA
KTSYTYNDLDRNTRVNDGSKNAYFEFDEFGNINVYTAGNGTAANYTYDST
QKVTNAAISSASGTQILDENYTYDAASNRTSIDNKQDGKTTYEYDAVNQL
TKETLPDGTVKAYTYDGFGNRTQVAISGSETKTIAASYNDGNQLVSWNGE
ALTYDANGNRTSDGKYTYTWDTGDRLSSITKKGESKPFTSYTYDDDNRRL
SKTVDGVTTNYHYDGDSIDVLYETDGDGKVVRQYVYSDDNVRLAMKMNGK
TLYYHYNAHGDVIALTDEAGKIVAEYAYDAWGNVLKNTASTEEAKANPYG
YAGYTYDKEIEQYYLMARYYEPEQGVFTAYDPDPGDEDDPQTMNGYNYAN
NNPVMMVDPDGNWAWLIPAAMAAGKFIYKFRKPIAKYGKKGAKWVGKTAK
KGAKWVGKTAKKGAKWARSRYNSKKKEFTVTKNWKVGLPKKSQGGRIFAI
INRKKGRLFALDYGKVGGNQKKYLHVHWNYKPKHYIIYPRKGRGK
ORF633 (SEQ ID NO:316)
MKKFILASLACTTLISFSPLVGQVEVSAATTNIATAPEKNLQEAPPATVD
QIFPDDALAFKVAQELGVSEDTVVTQEQLDTIETMVYVDFGVEDLTGMEY
LHNLKFVDLSQNKISNLENLANLTELETVSLNYNQITDITPLMNSPKLYN
LELGVNQISTLPSFENLTNLRILNLSSNQLKDISALKDTPQLTNLSISAN
NISDISVLSECDNLRVFYAESNQLTSIESLRNKTKLEYFDANFNQIKDVT
PLSTIPTIRSIKIEENQISDFSSLAGHRLELFDATGQNIYLPDVALGDST
NIVMKDNLGVTLHDWVWYTPGTYQNDTLTWENAGDNSAYFLNNQYPTYPS
VTVTVYQTVTP
ORF653 (SEQ ID NO:317)
MKKFSMRVVLIISVLFIAFGSANVSIAQERDTTNKLPEEELGSLDTSNII
AEEVAQEKPAEVEQLEEIPTTDELMQNPDVLEKSVADSDDPDLTVVSSGD
FWTIYRNTVNNEYNLRMFGNVPSSRPTAWNSYLKSIKHIEIEEATLTGSF
ASYFRGTDFPVLESVRIEQCNLSGVTSFEMAFYNSGIEKVIIRDNDYPKA
PSLLTATQYMFSLCRNLSELDLSGLDTSAVTNMYANFQNCTSLEELDLSN
FDTSSATNMYSMFSGCRSLEKLDVSNFDTSSVAHMQFMFEKCSILEELDL
SNFDTSSVTNMSYMFQLCPALKSLYLDNFTDAAIMTGMFAGTTSLTYLFV
SHNLSTFNNLENTSWYDEKNWVQFSNLSQLQTYHRQQSEPIGYRKGAFLS
LTMDAMGGEFEDAEEQKVQNKISGEYWEEVIPVKEGHYFDGWYLDQNFTN
KFDFSLPAAVSTTIYAKWVENYTVIIPASISLNEASELKVQGINRGSKTL
SVGLNYEETTISESNKLTLSNTADTTVRCLAPMSWDGSETNPEKAILTLA
PGSEITEGDAVMEIKSPENIQAGKYTGNVVFSIKYE
ORF660 (SEQ ID NO:318)
MLMSLSVLPLATYASETENTPTESYDGENFIATQTGNTLVIEDKKTGETV
KIEMNDEENGVITSDDGTIENVHRDEEGNVYVDNELELEAPPLDIEDGIN
IATQPRLLKASKWIYVQTTKYNTTTQGNMRSLALGILSFMPITGPIFGIV
AIIDAARSMGAKTLYVRVKQYRTSGYQFYKYDSYYYANASLTKLVKKTSQ
TKRNW
ORF72 (SEQ ID NO:319)
LKQCPVCENYTIEANYDICEVCYWEYDVVAQEYPDEIIGANNISLKQAKI
NYAKFCAVEEKYITLVRKPRQDELLE
ORF73 (SEQ ID NO:320)
MEYEQMADSLAYGEEYNFYYKNEEYWLSKNREGHYLTKVSDGETQEFRTS
EDLLGTARINGKLIIEIWENIQSQF
ORF74 (SEQ ID NO:321)
LKKLTTEQSFEYYLSSLCMLGLHTINLSDEEIEYEIFEELAIDYPAALSP
YTRELLVDNDIIDRELSLLSKQLQTKLFELDGGILW
ORF757 (SEQ ID NO:322)
MKNTVHDSEILSYHADFENSQLIMFVKDEENRKYKVIFEGLLTFCFEHQM
SNSIILDIVKGEVSSFISEKSILLSEGKNYFWPLDYESEDELLNYLNEKK
LNYYELQASYGLNGWILCSHYHIEL
ORF799 (SEQ ID NO:323)
MRQPFQVLVIPFIKTEANYQFGVLHRTDADVWQFVAGGGEDEEAISETA
KRESIEELNLDVDVKMYSLDSHASIPNFHFSFNKPYVVPEYCFAIDLTS
CSYQVTLSLEHSELRWVSYESAVQLLEWDSNKTALYELNERLKNNDMKA
M
ORF817 (SEQ ID NO:324)
MKKRLIGIITALLLVSGILLSPQMAQAETKVDYDALYQQGVSEGIINKAD
VSLETWIKENESEYNQVYQDGLKDGVYDASLSYEEWIKLNNYGQPPVVDE
NWEEVPQKPMLKGVYKGYNIKKGDILITNGTSSSGLLGHAAIANGNEYIL
DIPGAGKTTRQVPTSKWMSDYDPEGWVKVYRLKDSSVANAAANWADKNYY
STKGTSKQNIFPKYGMTGSRYSKNPTYCSKIVLQAYYFGTGNKPVVQVFP
SLVTVYDLPNYFSKAYKPQQVKYFK
ORF819 (SEQ ID NO:325)
MIGMFHYLLPLNQIPHNFHAQPGRISQMMHQWDFFHYFNRVSHCTGQGYL
FSRFCSPFCSASSRLIWEIDSPIALATRSILSRKICATTEVSSGIITLPN
CFSFYQSHLRSFQ
ORF949 (SEQ ID NO:326)
LKKLVKSAVVFASLVFIGTSATMITEKASAASIDPVQKADGQATYIPKGV
RDGTATEEHDGFEDGTNSVLQQVPLLRATTGYPDVNAYIKSNKFSTAKIE
KQLKSQFPKFNYRNGYGKPEGIVIHETANNSSTITGEINYMSTNYNNAFV
HAFVDKSRIIQIHPTENGVWGAGQYANARFIQVELVRSKTFDEFARSINN
YAYYAAYLLDQYNLPVDSAHSDGKGTVWSHDAVTRYLGGTTHTDPVSYFN
QWGYNFNSFVTLINEKYKAIQASKVTYDKIEYDKGVTAYARVKTAPGNAV
WTKPYRTEGSKLVNQLSVYQGKNNRILREAKTPITTWYQFSIDGKVIGWV
DTRALDTFYKQSMEQPANLTRYVASNKTGEAYYKVPVVDADVKWGTLAAY
KDQKLTVDKQATVEGQLWYRVRTSTTFIGWTKASNLTATSPFDKIEYDKG
VTAYARVKTAPGNAVWTKPYRTEGSKLVNQLSVYQGKNMRILREAKTVIT
TWYQFSIDGKVIGWVDTRALDTFYKQSMEKDTNLTRYVIANKVNEAYYKV
PVVDADVRWGTLAAYKDQKLTVDKQATVEGQLWYRVRTSTTFIGWTKASN
LTTTTPYDKIEYDKGATAYARVKTAPGNAVWTKPYRTEGSKLVNQLSVYQ
GKNNRILREAKTVITTWYQFSIDGKVIGWVDTRALDTFYKQSMEKDTNLT
RYVIANKVNEAYYKVPVVDADVRWGTLAAYKGQKLTVDKQATVEGQLWYR
IRTSSTFIGWTKASNLSATK

TABLE 5

Genes Specific for L. monocytogenes serovar 4b strain CLIP80459 Compared with
L. monocytogenes EGDe

| | | | |
|---|---|---|---|
| Gene | Gene category | Forward primer | Reverse primer |

| | | | |
|---|---|---|---|
| ORF1025 | 6 No similarity | GCGCTCGTCATGAAGATAAA | GCTATGTTTCCCATTCAACC |
| | | [SEQ ID NO:327] | [SEQ ID NO:328] |
| ORF 104 | 6 No similarity | AAAAATACTTGGCGGAATCTTA | CTTGCTCCGTTCTTCCAATA |
| | | [SEQ ID NO:329] | [SEQ ID NO:330] |
| [symbol] ORF 105 | 1.2 Transport/binding proteins | TGAATTAAAGCAAGGTGGGA | TGGCTTGCTAACAAAATCGT |
| | and lipoproteins | [SEQ ID NO:331] | [SEQ ID NO:332] |
| ORF 1063 | 1.8 Cell surface proteins | TACCAACCTTGCCCAAATTA | TAATCCACTTTTGCCACGTT |
| | | [SEQ ID NO:333] | [SEQ ID NO:334] |
| ORF 1227 | 1.2 Transport/binding proteins | TTGGAGTATGAGACGGTTTT | CCTTTTTCGTTAGAAGGACTTG |
| | and lipoproteins | [SEQ ID NO:335] | [SEQ ID NO:336] |
| ORF 1228 | 1.2 Transport/binding proteins | TCCCAAAAACTAATCCGAGC | TTTCACTTTCCGTCATTTCAA |
| | and lipoproteins | [SEQ ID NO:337] | [SEQ ID NO:338] |
| ORF 1272 | 6 No similarity | AAATCCAATAAAACTAGCAATAAGG | GCAGTGAAAATTTGTTCAGGA |
| | | [SEQ ID NO:339] | [SEQ ID NO:340] |
| ORF 1384 | 6 No similarity | CCGGACTTTGCTTATCAATTT | GTTCTTGCAATTCAGGAGGA |
| | | [SEQ ID NO:341] | [SEQ ID NO:342] |
| ORF 1385 | 6 No similarity | TTGAAGAACAGGTTTCGGAG | CAATATCAAAATCTTCCGCAT |
| | | [SEQ ID NO:343] | [SEQ ID NO:344] |
| ORF 1386 | 6 No similarity | CAATAATTTTAAAGGAGCGTAAC | CCGAGCAAGATTGCAAGTA |
| | | [SEQ ID NO:345] | [SEQ ID NO:346] |
| ORF 1387 | 6 No similarity | ACAATTCCTAACAAAACAGCTCA | AATGCGACAAATTTCCAATC |
| | | [SEQ ID NO:347] | [SEQ ID NO:348] |
| ORF 1388 | 6 No similarity | no primer, too small | no primer, too small |
| ORF 1389 | 6 No similarity | ATTTTAATTTGCATCGAGCG | TACGCAAGTAACATAATCGCC |
| | | [SEQ ID NO:349] | [SEQ ID NO:350] |
| [symbol] ORF 1390 | 1.2 Transport/binding proteins | TGAATCAAATAACCCAAATTGA | CTGGCGAATGAGTAGCAAAT |
| | and lipoproteins | [SEQ ID NO:351] | [SEQ ID NO:352] |
| ORF 1391 | 6 No similarity | GGGGGTAAACCACAAGTTTT | CCACAAATCAAATCAAACACA |
| | | [SEQ ID NO:353] | [SEQ ID NO:354] |
| ORF 1400 | 1.1 Cell wall | ATCGAGAAGCGGGAATAAGT | TAGAGTTTCGCACAAGGACC |
| | | [SEQ ID NO:355] | [SEQ ID NO:356] |
| [symbol] ORF 1445 | 5.2 From other organisms | TCCTGCTGGTTTTATTACGG | ATCAACTACGACTGCCCAAG |
| | | [SEQ ID NO:357] | [SEQ ID NO:358] |
| ORF 149 | 1.8 Cell surface proteins | AGGACCGGGAAAATCACTAA | CACCATAGCAGTGTTTGAAGG |
| | | [SEQ ID NO:359] | [SEQ ID NO:360] |
| [symbol] ORF 1490 | 1.2 Transport/binding proteins | TTGGACCAGGAGTTGTGAAC | CAATGTTATCCCAGCGATTC |
| | and lipoproteins | [SEQ ID NO:361] | [SEQ ID NO:362] |
| [symbol] ORF 1491 | 5.2 From other organisms | CTTGGCGAAAACGACTTAAA | CATGACAATGGCAACTTTGA |
| | | [SEQ ID NO:363] | [SEQ ID NO:364] |
| ORF 1492 | 3.5 Regulation | AACCGCAAAAGAAAGGCTAT | TTTCGTCGTAGTGATGGGAT |
| | | [SEQ ID NO:365] | [SEQ ID NO:366] |
| ORF 150 | 1.8 Cell surface proteins | AAAAGGGATGATTAGTTTAATTGC | GCCAACCAATAATTTTGTCTC |
| | | [SEQ ID NO:367] | [SEQ ID NO:368] |
| ORF 1504 | 1.8 Cell surface proteins | TGACTGAAGCGAATCTTGAA | TGGATTTAACCAAGCATACGA |
| | | [SEQ ID NO:369] | [SEQ ID NO:370] |
| [symbol] ORF 1590 | 1.8 Cell surface proteins | CATCTGACGTTTCACAAGCA | GCATAATTTCCATAGCCCCT |
| | | [SEQ ID NO:371] | [SEQ ID NO:372] |
| ORF 1654 | 2.1.1 Specific pathways | AATTTTCCGGATAACTTGCC | AATTTTCCGGATAACTTGCC |
| | | [SEQ ID NO:373] | [SEQ ID NO:374] |
| ORF 1655 | 3.5 Regulation | GAGGCAGAAACTGGATTTGA | AAGTTTGTTGTTCCGCATGT |
| | | [SEQ ID NO:375] | [SEQ ID NO:376] |
| ORF 1680 | 1.2 Transport/binding proteins | TTACTTGTTTCGATTGGGGA | TTTAAAAAGTCGTTCTGCGG |
| | and lipoproteins | [SEQ ID NO:377] | [SEQ ID NO:378] |
| ORF 1681 | 1.2 Transport/binding proteins | GTGAATGCACCATAAAGGCT | ATTCGTTCTGCTTGATTTGC |
| | and lipoproteins | [SEQ ID NO:379] | [SEQ ID NO:380] |
| ORF 1683 | 5.2 From other organisms | TGCATCGGTTGGCTATTATT | TGTTTGCCTACTTCAATCCAA |
| | | [SEQ ID NO:381] | [SEQ ID NO:382] |
| ORF 1684 | 3.5 Regulation | TTAGGCGAGCGATTATCAAC | GCAATCAAAATCATTGCGTT |
| | | [SEQ ID NO:383] | [SEQ ID NO:384] |
| ORF 1717 | 1.8 Cell surface proteins | TCCATGTTTGCTGCTTGTAA | CAAGTGAGCTGCACTCAAAA |
| | | [SEQ ID NO:385] | [SEQ ID NO:386] |
| ORF 1718 | 3.5 Regulation | GGTAGAAATAACGGGGGATG | ACTCGTTCACTGGCAAAATC |
| | | [SEQ ID NO:387] | [SEQ ID NO:388] |
| ORF 1739 | 1.1 Cell wall | TTAATGATGGAAGCACGGAT | TTAATGATGGAAGCACGGAT |
| | | [SEQ ID NO:389] | [SEQ ID NO:390] |
| ORF 1813 | 5.2 From other organisms | TTCTGGTGGGTATAAAGGAAAA | CATTCCTTCCAATGACCTTT |
| | | [SEQ ID NO:391] | [SEQ ID NO:392] |
| ORF 1849 | 1.8 Cell surface proteins | CTGCAATCAGCCAGATTTTT | GCGCTTGTAGCTCATCTAGG |
| | | [SEQ ID NO:393] | [SEQ ID NO:394] |
| ORF 1912 | 5.1 From Listeria | ACAAATCTTTCCGGTGATGA | TTAGCAGCTGCAATAGGGTC |
| | | [SEQ ID NO:395] | [SEQ ID NO:396] |
| ORF 1913 | 5.1 From Listeria | CCCGGTTAGAACAGCAACTA | TCATTGGTTCCACTCCTTTC |
| | | [SEQ ID NO:397] | [SEQ ID NO:398] |
| ORF 1998 | 3.2 DNA restriction/modification | AAGCGTACCAGTAATCCGGT | GAGCATTAATTCCTGGTTGG |
| | and repair | [SEQ ID NO:399] | [SEQ ID NO:400] |
| ORF 1999 | 3.2 DNA restriction/modification | GGGAAAATTGGGAGAGTGTT | CGCGTCTACCTGAGTTCTTC |
| | and repair | [SEQ ID NO:401] | [SEQ ID NO:402] |
| ORF 2013 | 1.8 Cell surface proteins | GCAGGCGAAACATATGAAAC | CTATCGCGCAACGATCTAAT |
| | | [SEQ ID NO:403] | [SEQ ID NO:404] |
| [symbol] ORF 2017 | 1.8 Cell surface proteins (2 | AACGGTTTTTCAAGCATTTG | CAGAGTCCACACCTGTTGGT |
| | sondes) | [SEQ ID NO:405] | [SEQ ID NO:406] |
| ORF 2019 | 1.8 Cell surface proteins | GATTATCCGACAGCACCATC | CAACAAACATGGCCAACATA |
| | | [SEQ ID NO:407] | [SEQ ID NO:408] |
| [symbol] ORF 2074 | 1.1 Transport/binding proteins | TTCTCTCTGGAATGAGTCCG | TGAGAAGACGGCACCTGTAT |
| | and lipoproteins | [SEQ ID NO:409] | [SEQ ID NO:410] |
| ORF 2075 | 1.1 Transport/binding proteins | TTGACAGCTTTTAGAGCGGA | ATACTTAATTTCGTCGCCCC |
| | and lipoproteins | [SEQ ID NO:411] | [SEQ ID NO:412] |
| [symbol] ORF 2076 | 1.1 Transport/binding proteins | CAAGGTATGGCGACATTAGG | TAAAAGCGCCCAAATAAGTG |
| | and lipoproteins | [SEQ ID NO:413] | [SEQ ID NO:414] |
| [symbol] ORF 2110 | 5.2 From other organisms | TTAGGTGGAGGAATTGTTGC | TTCGTGCCATTATATCCAGG |
| | | [SEQ ID NO:415] | [SEQ ID NO:416] |
| ORF 2192 | 3.2 DNA restriction/modification | GCAGAAGCAAAATTTGAAGC | TCCGTCACCATGATAACCTT |
| | and repair | [SEQ ID NO:417] | [SEQ ID NO:418] |
| ORF 2217 | 3.2 DNA | CGATGAAAAATGATGAAGGG | ATCTGAAAGAAGATGGCGTG |
| | restriction/modification and | [SEQ ID NO:419] | [SEQ ID NO:420] |
| | repair | | |
| ORF 2247 | 5.1 From Listeria | CATCCGCTTATGTGGCTAGT | CGTTTCACATGGTAGGTCGT |
| | | [SEQ ID NO:421] | [SEQ ID NO:422] |
| ORF 2291 | 1.8 Cell surface proteins | no primer | no primer |
| ORF 2293 | 6 No similarity | no primer | no primer |
| ORF 2336 | 5.1 From Listeria | no primer | no primer |
| [symbol] ORF 2372 | 1.1 Cell wall | CGAGGACAGATTTGAAAGGA | GCAAAATTGCTTTCTGTGGT |
| | | [SEQ ID NO:423] | [SEQ ID NO:424] |
| ORF 2375 | 1.8 Cell surface proteins | no primer | no primer |
| ORF 2440 | 5.2 From other organisms | CCACATCCAATTGCATAACA | CCAATTACGGTGGTTCTCAT |
| | | [SEQ ID NO:425] | [SEQ ID NO:426] |
| ORF 2462 | 1.1 Cell wall | TGTCGTGTCTATTGATGCTGA | GCCAATTAACCACAAGGAAA |
| | | [SEQ ID NO:427] | [SEQ ID NO:428] |
| [symbol] ORF 2541 | 1.8 Cell surface proteins | TAACAACCATTGTCCCTTGG | AACCAATCAGCAGGAATGTC |
| | | [SEQ ID NO:429] | [SEQ ID NO:430] |
| ORF 2568 | 1.8 Cell surface proteins | CTTTAGTTTTCCAGGCACCA | CACCCGTTTTCACTGTATCC |
| | | [SEQ ID NO:431] | [SEQ ID NO:432] |
| ORF 2646 | 5.1 From Listeria | ACGGTCTTTCATCTAACCCC | TTTGTTGGGTAGCCATTGAT |
| | | [SEQ ID NO:433] | [SEQ ID NO:434] |
| ORF 2666 | 6 No similarity | AAATGCACTGCACCAATCTT | AAATACTCCAACGGGTCCTC |
| | | [SEQ ID NO:435] | [SEQ ID NO:436] |
| ORF 2758 | 6 No similarity | AATGCCAAAAGAATCGAAAC | AAGCGGATTTCATATTGCTG |
| | | [SEQ ID NO:437] | [SEQ ID NO:438] |
| [symbol] ORF 2759 | 2.1.1 Specific pathways | CATTTCTCGCGAATCACTCT | TAGCTAAACGTTCCACTCGG |
| | | [SEQ ID NO:439] | [SEQ ID NO:440] |
| ORF 2763 | 1.1 Cell wall | CAGGCGGTAATAATAAGCGA | GCCAGTCATCTGCCATATTT |
| | | [SEQ ID NO:441] | [SEQ ID NO:442] |
| [symbol] ORF 2777 | 5.2 From other organisms | GCCGAGGTCATATCACTCAA | TGGAAGTATCATCAGAAACCAA |
| | | [SEQ ID NO:443] | [SEQ ID NO:444] |
| ORF 2778 | 1.2 Transport/binding proteins | TTGGTTCACACAAAACATGG | AAAAATGAGTAGCAGTGAGCA |
| | and lipoproteins | [SEQ ID NO:445] | [SEQ ID NO:446] |
| ORF 2779 | 1.2 Transport/binding proteins | TTTTACTTCTTGCTACTTGTTCTCT | CAAGTATAGGATAAATGCTACCGC |
| | and lipoproteins | [SEQ ID NO:447] | [SEQ ID NO:448] |
| ORF 2780 | 5.2 From other organisms | TTTGACTCCCGTTCAAAAC | TCCAGTTCTAAAGCCCATACC |
| | | [SEQ ID NO:449] | [SEQ ID NO:450] |
| ORF 2815 | 5.2 From other organisms | AAGCGTGATGAAGAATTGGA | ATCCGCTAGGGAAGCTTTAT |
| | | [SEQ ID NO:451] | [SEQ ID NO:452] |
| ORF 2817 | 6 No similarity | CTGTTTCCATTGGTATCGCT | GCTCCATTGGCTCTTAAGGT |
| | | [SEQ ID NO:453] | [SEQ ID NO:454] |
| [symbol] ORF 2819 | 5.2 From other organisms | TTTCAAGGAGGAGCAAAATG | CACTAAAGCCTCCCATTGAG |
| | | [SEQ ID NO:455] | [SEQ ID NO:456] |
| ORF 2836 | 1.8 Cell surface proteins | TGGTGAAATGGAGAGGTTTT | TTTCCAGTTCCATTTTCCAC |
| | | [SEQ ID NO:457] | [SEQ ID NO:458] |
| [symbol] ORF 2847 | 6 No similarity | ACAACCGGCCAATTAAGACT | TGCAAATACTGCGCTGATAG |
| | | [SEQ ID NO:459] | [SEQ ID NO:460] |
| ORF 2848 | 6 No similarity | CGAAAGGAACCACATGTATGA | GCTCTTTTTGTATAGGAAATGTAAT |
| | | [SEQ ID NO:461] | [SEQ ID NO:462] |
| ORF 2854 | 5.1 From Listeria | AATCACAGATGGGCCAATTA | ATATAGGAGCGCAGAGTTGC |
| | | [SEQ ID NO:463] | [SEQ ID NO:464] |
| ORF 2884 | 6 No similarity | no primer | no primer |
| ORF 2885 | 6 No similarity | no primer | no primer |
| ORF 2886 | 6 No similarity | TTGAAATTGCTGCTGATTATGT | ATCTTGTTCTACACTGCCGC |
| | | [SEQ ID NO:465] | [SEQ ID NO:466] |
| ORF 2887 | 6 No similarity | ATGCTTGACAAACAACAACTAT | GCGCCAAATCTATTCCTTTT |
| | | [SEQ ID NO:467] | [SEQ ID NO:468] |
| ORF 29 | 1.8 Cell surface proteins | TAACTATTGGCGATGGTGCT | AATCGATTTGTCCCAGTGAA |
| | | [SEQ ID NO:469] | [SEQ ID NO:470] |
| ORF 2925 | 5.1 From Listeria | AAAACAAGGCGTTATGTGGA | CCCAATCCCACTCATGTCTA |
| | | [SEQ ID NO:471] | [SEQ ID NO:472] |
| ORF 2927 | 5.2 From other organisms | no primer | no primer |
| ORF 2969 | 6 No similarity | ATCCAATTCGAAAGGGACTC | TGTTTGATTAATTCTGTGGCA |
| | | [SEQ ID NO:473] | [SEQ ID NO:474] |
| ORF 2970 | 6 No similarity | GGATGTGAAAATGGGGATAC | CTATTGGCACTCCCGTATTG |
| | | [SEQ ID NO:475] | [SEQ ID NO:476] |
| ORF 3102 | 6 No similarity | AGATGTCGTTGAACAGAGGG | TTGGTTGGAGTGTCCAATTT |
| | | [SEQ ID NO:477] | [SEQ ID NO:478] |
| [symbol] ORF 3103 | 6 No similarity | TGGCAAAAGAAATTAAGGTAAA | GTTTGTCCCAACTGACCATC |
| | | [SEQ ID NO:479] | [SEQ ID NO:480] |
| ORF 3132 | 6 No similarity | ACCGATTATTTGCAACCTCT | TGACGTTCTTTTTATCGTAAAGC |
| | | [SEQ ID NO:481] | [SEQ ID NO:482] |
| ORF 3135 | 6 No similarity | TACTTTACAATTCACAATGGGATTA | ACAACAAATATTGGAATTCTGAAAC |
| | | [SEQ ID NO:483] | [SEQ ID NO:484] |
| ORF 3136 | 6 No similarity | TCCAGTGATGGAAATGTGATT | ACCTCTGTTTTAATGACGCCT |
| | | [SEQ ID NO:485] | [SEQ ID NO:486] |
| ORF 3137 | 6 No similarity | GGAGACGCTGAAAAGACAGA | TTTTTCGGGTTCTCTTCTGA |
| | | [SEQ ID NO:487] | [SEQ ID NO:488] |
| ORF 3138 | 6 No similarity | TCCAATGCCAGATAAAACAGTA | TCAATTTCTTCCACACTTTTACA |
| | | [SEQ ID NO:489] | [SEQ ID NO:490] |
| ORF 3140 | 5.2 From other organisms | GGCACTAGTTATTGGAGTATTGG | ACCAAGTTTCCTCATCCCA |
| | | [SEQ ID NO:491] | [SEQ ID NO:492] |
| ORF 3141 | 1.2 Transport/binding proteins | TACTTAGATCGCTTGCTGGC | AATTTTCCCTTGGTCCATTC |
| | and lipoproteins | [SEQ ID NO:493] | [SEQ ID NO:494] |
| ORF 3142 | 5 From other organsisms | TCCTCGTTCCTAAAGGTATTCA | TATATGCCGGTTTGGTTTCA |
| | | [SEQ ID NO:495] | [SEQ ID NO:496] |
| ORF 3144 | 6 No similarity | TGTCTAACAAAGCGGAAGATG | TGTAGTTATTGTCGCAGGGA |
| | | [SEQ ID NO:497] | [SEQ ID NO:498] |
| ORF 3172 | 5.2 From other organsims | TGATGTTAGAGGCGAGTTTGA | TAATTCCTCTGCAGTTGGCT |
| | | [SEQ ID NO:499] | [SEQ ID NO:500] |
| ORF 3173 | 6 No similarity | AGTCAAAATCACAAGCGAGC | CTTCTGCTTGAGCACTCCAT |
| | | [SEQ ID NO:501] | [SEQ ID NO:502] |
| ORF 3174 | 6 No similarity | TGGCTTATTGATTCACTGGC | TTTGCCGTATATCGAGGAGT |
| | | [SEQ ID NO:503] | [SEQ ID NO:504] |
| ORF 3175 | 6 No similarity | no primer | no primer |
| ORF 3183 | 6 No similarity | CAGCAGGCCTTTATGGTAGA | CTTTCCTCTCCCAACAACACT |
| | | [SEQ ID NO:505] | [SEQ ID NO:506] |
| [symbol] ORF 3184 | 5.2 From other organisms | TGGGAAACTTTGAGTGGAAA | TTGCAAAGGCATCCATACTT |
| | | [SEQ ID NO:507] | [SEQ ID NO:508] |
| ORF 3185 | 6 No similarity | CGCAGACATTTGACAATGAT | CCAGTTTCGCCATGTTCTAT |
| | | [SEQ ID NO:509] | [SEQ ID NO:510] |
| ORF 3222 | 6 No similarity | no primer | no primer |
| ORF 3224 | 1.8 Cell surface proteins | GATTTGCCTTTATCCCTGGT | TGGTTACGTGGATGTCCTTT |
| | | [SEQ ID NO:511] | [SEQ ID NO:512] |
| ORF 3277 | 6 No similarity | GCTTGGAATGCTTTTGATA | TTGAAGCATGATAAGTGCCA |
| | | [SEQ ID NO:513] | [SEQ ID NO:514] |
| ORF 3278 | 6 No similarity | TTTTGAGCCCGTTCTGTTTA | CAATAATCCCACCAACCAAG |
| | | [SEQ ID NO:515] | [SEQ ID NO:516] |
| ORF 3279 | 1.2 Transport/binding proteins | CAACAAATAACTGTCGCACG | CCAATCAAAGCAAACAATCC |
| | and lipoproteins | [SEQ ID NO:517] | [SEQ ID NO:518] |
| ORF 3283 | 6 No similarity | no primer | no primer |
| ORF 3291 | 5.2 From other organsims | ACCTATTGGAAGCGTTGTTT | ATCTCATCCTCCTCTTCTGGA |
| | | [SEQ ID NO:519] | [SEQ ID NO:520] |
| ORF 3316 | 5.2 From other organsisms | GCAAAAATAATTTCAGAAAATCC | TTCCAAATACCAAAAATCAGAAA |
| | | [SEQ ID NO:521] | [SEQ ID NO:522] |
| ORF 3324 | 4.5 Transposon and IS | no primer | no primer |
| ORF 3325 | 6 No similarity | no primer | no primer |
| ORF 3326 | 6 No similarity | no primer | no primer |
| ORF 3327 | 1.8 Cell surface proteins | CGTCTTTTAGAACCGCATTT | TCATAATTGCTGCATCCGTA |
| | | [SEQ ID NO:523] | [SEQ ID NO:524] |
| ORF 3361 | 6 No similarity | TTCAATCAAATAAAGGAGCGTAT | CGATATTGTATTCCTTACCTAATGT |
| | | [SEQ ID NO:525] | [SEQ ID NO:526] |
| ORF 3362 | 6 No similarity | GATTATTCTAAAATGCCATGGGTA | ATGGATCATCTATATTATCTCCGA |
| | | [SEQ ID NO:527] | [SEQ ID NO:528] |
| ORF 3365 | 3.5 Regulation | GAAAAAGTAAAAGAAATGATCCGA | TTTGTTGGTAGCCATTTTCA |
| | | [SEQ ID NO:529] | [SEQ ID NO:530] |
| ORF 3378 | 1.1 Cell wall | AGGGAGTGGAGAAAAAGAGTG | AGGTGAGTCGTAAATCGCAC |
| | | [SEQ ID NO:531] | [SEQ ID NO:532] |
| ORF 3379 | 1.1 Cell wall | CGCGGATATAGATGCTGATT | ACAAGCACAAACAAAGAGGC |
| | | [SEQ ID NO:533] | [SEQ ID NO:534] |
| ORF 3386 | 1.2 Transport/binding proteins | ATCTGAATCTGGACGAGCAC | CCGGATCTAAGGGCACTAAT |
| | and lipoproteins | [SEQ ID NO:535] | [SEQ ID NO:536] |
| [symbol] ORF 3401 | 5.1 From Listeria | TGCATAAGCATTTTTAAACCG | CCAAATAATGGCACACAGAA |
| | | [SEQ ID NO:537] | [SEQ ID NO:538] |
| ORF 3402 | 1.1 Cell wall | GCAGCAACCTATAAAGGCAA | GTCTTAGCCTCGCGTATCAA |
| | | [SEQ ID NO:539] | [SEQ ID NO:540] |
| ORF 3410 | 5.2 From other organsisms | ACAACCAAAGAAGCGACAAC | GTAAGAAGCCCACGAGCTG |
| | | [SEQ ID NO:541] | [SEQ ID NO:542] |
| ORF 3457 | 4.6 Miscellaneous | GTGTCAACGACGAAAAACAA | CGATGTAGTGCTTTCATGTCA |
| | | [SEQ ID NO:543] | [SEQ ID NO:544] |
| ORF 3501 | 6 No similarity | AATCCTGAGTCAATGAAAGCA | AGTCCTGTTTTAGGATCAATCG |
| | | [SEQ ID NO:545] | [SEQ ID NO:546] |
| ORF 3593 | 5.2 From other organisms | GCAGATGATTTTGGGTTCAC | AAACTCGACATGCTGAAAGC |
| | | [SEQ ID NO:547] | [SEQ ID NO:548] |
| ORF 3594 | 1.2 Transport/binding proteins | GATGCCTCTTCCTCGTTTCT | ACTTTTCATCCAAACGCTTA |
| | and lipoproteins | [SEQ ID NO:549] | [SEQ ID NO:550] |
| ORF 3745 | 4.3 Phage related functions | GCGCTATTTTAGATGCGGTA | AAGATGCATGGGTATGTCGT |
| | | [SEQ ID NO:551] | [SEQ ID NO:552] |
| [symbol] ORF 3792 | 6 No similarity | GGGCTAAAAATAATAATTGGCA | CACTATTTCTCCAGCCACGA |
| | | [SEQ ID NO:553] | [SEQ ID NO:554] |
| ORF 3793 | 5.2 From other organisms | AGTGAAAATGCGGACTATGG | CTGACTTTCATTAAAATCTTGCC |
| | | [SEQ ID NO:555] | [SEQ ID NO:556] |
| [symbol] ORF 3840 | 5.1 From Listeria | GAAACAAATGGACGACTGGT | TGGCATAGCTGCTGTAATGA |
| | | [SEQ ID NO:557] | [SEQ ID NO:558] |
| ORF 3913 | 5.2 From other organisms | TGAGAAGGCAGAAAATTTGA | TCCAAGCTTCTAATCACCGT |
| | | [SEQ ID NO:559] | [SEQ ID NO:560] |
| ORF 3914 | 6 No similarity | TTATTTTGTACGGGCGTTTT | ACCTGCGCACAATCTTCTT |
| | | [SEQ ID NO:561] | [SEQ ID NO:562] |
| ORF 3921 | 3.2 DNA restriction/modification | TGCAAATCGACAAGTAACCA | GAACCGTCATTACGCTCATT |
| | and repair | [SEQ ID NO:563] | [SEQ ID NO:564] |
| ORF 3970 | 5.2 From other organisms | no primer | no primer |
| ORF 3981 | 6 No similarity | CAAAGGCAGCAAATTAGAAGA | GCAAAAGTGTTTGGGCATA |
| | | [SEQ ID NO:565] | [SEQ ID NO:566] |
| ORF 4017 | 1.1 Cell Wall | GGCTGTTTTTGAAGCTTACG | GTGAAATCAAAATGTTCCGC |
| | | [SEQ ID NO:567] | [SEQ ID NO:568] |
| ORF 4018 | 1.1 Cell wall | AGTGGATGTGACCGGTTTTA | CCGAGTGCTTGTTTCATCTT |
| | | [SEQ ID NO:569] | [SEQ ID NO:570] |
| ORF 4100 | 1.2 Transport/binding proteins | AATCTGGCTTGGTGAAAGTG | GTGCGCAGGATAAGATGATT |
| | and lipoproteins | [SEQ ID NO:571] | [SEQ ID NO:572] |
| ORF 4101 | 1.2 Transport/binding proteins | CTAGGAGACAATGGAGCTGG | AACTTTCGTTGCCCAGTTTT |
| | and lipoproteins | [SEQ ID NO:573] | [SEQ ID NO:574] |
| ORF 4132 | 1.8 Cell surface proteins | GTCGGGATAATGTTCTTTCAA | CAATGGTTGTTAGTTTCGGG |
| | | [SEQ ID NO:575] | [SEQ ID NO:576] |
| ORF 4133 | 4.3 Phage related functions | GTGCTTTTACAACAGCTCGG | CTAATATTATTGCCTTCATTTTCA |
| | | [SEQ ID NO:577] | [SEQ ID NO:578] |
| ORF 4135 | 4.3 Phage related functions | AAAGAGAATGTTATCCGATGCT | TCATTCCATACCAATTTCATGT |
| | | [SEQ ID NO:579] | [SEQ ID NO:580] |
| ORF 4188 | 3.5 Regulation | AGCAGAGAAAACGGGCTATT | CAGTACAGTAGGCAAGGCGT |
| | | [SEQ ID NO:581] | [SEQ ID NO:582] |
| ORF 4327 | 6 No similarity | TGAATTATGATGAACGTGTACGA | TAAACGTGTTTTTACGCTGC |
| | | [SEQ ID NO:583] | [SEQ ID NO:584] |
| ORF 4369 | 6 No similarity | GAGAGCGAACTAGAGGGAGG | CCAGAAGTTTGGTTCACACC |
| | | [SEQ ID NO:585] | [SEQ ID NO:586] |
| ORF 4457 | 6 No similarity | AGGGATAAAGATTTCAGGGAT | TGTTGTTGCTGCCTACACAT |
| | | [SEQ ID NO:587] | [SEQ ID NO:588] |
| ORF 4507 | 5.2 From other organisms | AGGAAACAGCAAAATGATCG | ATGCAAGCTATGCGTGTATG |
| | | [SEQ ID NO:589] | [SEQ ID NO:590] |
| ORF 4563 | 1.2 Transport/binding proteins | ATACACATGAATGATGGCACA | TCATTTTGCTGTTTCCTTTCT |
| | and lipoproteins | [SEQ ID NO:591] | [SEQ ID NO:592] |
| ORF 4578 | 6 No similarity | GCGGGTGCAATATTTTATC | CCAAAGGCCTGTAGCAGTAA |
| | | [SEQ ID NO:593] | [SEQ ID NO:594] |
| ORF 484 | 1.2 Transport/binding proteins | AGGCAGTACGTCAAGGAATG | AAAGAACGTAAAATCGGCGT |
| | and lipoproteins | [SEQ ID NO:595] | [SEQ ID NO:596] |
| ORF 485 | 1.2 Transport/binding proteins | TTTGGCGGATTATCCACTT | TTTCCCTCCGTCTTAAATGT |
| | and lipoproteins | [SEQ ID NO:597] | [SEQ ID NO:598] |
| ORF 577 | 1.8 Cell surface proteins | TTTTAATAACCACGACGGGA | AACATTCTCGGATAACGCTG |
| | | [SEQ ID NO:599] | [SEQ ID NO:600] |
| ORF 633 | 1.9 Soluble Internalins | AGTCGCACAGGAACTAGGTG | GATTGCTTTCGGCATAAAAA |
| | | [SEQ ID NO:601] | [SEQ ID NO:602] |
| ORF 653 | 1.8 Cell surface proteins | TTGATTTGAGTGGCCTTGAT | ACTGCACCCAATTCTTTTCA |
| | | [SEQ ID NO:603] | [SEQ ID NO:604] |
| ORF 660 | 6 No similarity | ATGAATGACGAAGAAAACGG | CCACATTCGTTTAGTCTGGG |
| | | [SEQ ID NO:605] | [SEQ ID NO:606] |
| [symbol] ORF 72 | 5.2 From other organisms | GGGTTTTTCACAACAAGTGG | |
| | | [SEQ ID NO:607] | |
| ORF 73 | 6 No similarity | | TCCCTCTATCCGTTCAATCA |
| | | | [SEQ ID NO:608] |
| ORF 74 | 6 No similarity | TTCGCTATGTATGTTAGGATTGC | CCACAGAATTCCTCCATCAA |
| | | [SEQ ID NO:609] | [SEQ ID NO:610] |
| [symbol] ORF 757 | 6 No similarity | AGCGAAATATTGTCTTATCATGC | AGTGAGAACATAATATCCAACCGT |
| | | [SEQ ID NO:611] | [SEQ ID NO:612] |
| [symbol] ORF 799 | 6 No similarity | CGTTTATTAAAACGGAAGCA | TTGTTTTTCAACCGTTCATTT |
| | | [SEQ ID NO:613] | [SEQ ID NO:614] |
| [symbol] ORF 817 | 5.2 From other organsims | TGACGCATTGTATCAACAGG | TTTTGTTTGGATGTGCCTTT |
| | | [SEQ ID NO:615] | [SEQ ID NO:616] |
| ORF 819 | 6 No similarity | TGATTGGAATGTTCCACTATT | AAATGAAAAACAGTTCGGTAGTG |
| | | [SEQ ID NO:617] | [SEQ ID NO:618] |
| ORF 949 | 1.8 Cell surface proteins | GTCGAAGGACAGCTTTGGTA | CAGCTAAAGTGCCCCATCTA |
| | | [SEQ ID NO:619] | [SEQ ID NO:620] |

Gene names in bold type were used to construct the macroarray described in Example 1. Gene names flanked by a bar were used to generate a second array comprising 192 genes.

TABLE 6
L. monocytogenes (CLIP 80459) specific genes

| Gene | Description | Functional category |
|---|---|---|
| ORF 2541 | unknwon, similar to internalin proteins, putative peptidoglycan bound | cell surface proteins |
| | protein (LPXTG motif) | |
| ORF 2017 | unknwon, similar to internalin proteins, putative peptidoglycan bound | cell surface proteins |
| | protein (LPXTG motif) | |
| ORF 3401 | unknown | unknown |
| ORF 2074 | unknown similar to oligo-1,6-glucosidase | Specific pathways |
| ORF 0528-0529 | unknown—unknown | unknown |
| ORF 2815 | unknown, similar to heat shock protein HtpG | unknown |
| ORF 2568-2569 | unknwon, internalin-like, putative peptidoglycan bound protein (LPXTG | cell surface proteins |
| | motif)-unknown | |
| ORF 2847 | similar to phage integrase | Phage-related functions |
| ORF1388-1387 | unknown—unknown | unknown |
| ORF 0799 | unknown | unknown |
| ORF 72-73-74 | unknown—unknown—unknown | unknown |
| ORF1912-1913 | unknown—unknown | unknown |
| ORF 0150 | unknown, similar to amidase | cell wall |
| ORF 2777 | unknown | unknown |
| ORF 2579 | unknown, sucrose phosphorylase | Specific pathways |
| ORF 0075 | unknown | unknown |
| ORF 660 | unknown | unknown |
| ORF 2884-2885 | unknown | unknown |
| ORF 2885 | unknown | unknown |
| ORF 1445 | unknown, conserved hypothetical protein ykcB - Bacillus subtilis | unknown |
| ORF 1590 | unknwon, internalin-like, putative peptidoglycan bound protein (LPXTG | cell surface proteins |
| | motif) | |
| ORF 0182 | unknown, similar to B. subtilis YukA, | unknown |
| ORF 1761 | unknwon, internalin-like, putative peptidoglycan bound protein (LPXTG | cell surface proteins |
| | motif) | |
| ORF 0105 | unknown, similar to similar to ABC transporter | Transport/binding proteins and |
| | | lipoproteins |
| ORF 2372 | unknown, similar to teichoic acid biosynthesis protein C | cell wall |
| ORF 2110 | unknown, similar to hypothetical protein | unknown |
| ORF 1491 | unknown, similar to hypothetical protein | unknown |
| ORF 1490 | unknown, similar to sucrose-specific PTS system IIBC component | Transport/binding proteins and |
| | | lipoproteins |
| ORF 1390 | unknown, similar to hypothetical protein | unknown |
| ORF 1391 | unknown | unknown |
| ORF 1228 | unknown, similar to similar to ABC transporter | Transport/binding proteins and |
| | | lipoproteins |
| ORF 0029 | unknwon, internalin-like, putative peptidoglycan bound protein (LPXTG | cell surface proteins |
| | motif) | |
| ORF 3792 | unknown | unknown |
| ORF 3840 | unknown, similar to transcription regulator | Regulation |
| ORF 4101 | unknown, similar to similar to ABC transporter | Transport/binding proteins and |
| | | lipoproteins |
| ORF 3103 | unknown, similar to peptidoglycan lytic protein P45 | cell wall |
| ORF 1490 | unknown, similar to sucrose phosphotransferase enzyme IIBC | Transport/binding proteins and |
| | component | lipoproteins |
| ORF 2217 | unknown, imilar to type II DNA modification enzyme | DNA restriction/modification and |
| | | repair |
| ORF 2939-2968 | unknown—unknown | unknown |
| ORF 4457 | unknown, DNA double-strand break repair rad50 ATPase | DNA restriction/modification and |
| | | repair |
| ORF 3184 | unknown | unknown |
| ORF 0148-0149 | unknown-unknown, similar to amidase | cell wall |
| ORF 3157 | unknown, similar to phage protein | Phage-related functions |
| ORF 2858 | unknown, similar to hypothetical protein | unknown |
| ORF 1384-1385 | unknown—unknown | unknown |
| ORF 2541 | unknwon, internalin-like, putative peptidoglycan bound protein (LPXTG | cell surface proteins |
| | motif) | |
| ORF 2819 | unknown, similar to transcriptional regulator | Regulation |
| ORF 2262 | unknown | unknown |
| ORF 0817 | unknown | unknown |
| ORF 0757 | unknown | unknown |
| ORF 2013 | unknwon, internalin-like, putative peptidoglycan bound protein (LPXTG | cell surface proteins |
| | motif) | |
| ORF 2076 | unknown, similar to maltodextrin ABC transporter | Transport/binding proteins and |
| | | lipoproteins |
| ORF 2013 | unknwon, internalin-like, putative peptidoglycan bound protein (LPXTG | cell surface proteins |
| | motif) | |

TABLE 7

List of the Genes and Probe Primers on the Macroarray

Spot Label

Spot Lm 4b

| | | |
|---|---|---|
| ORF 2541 | TACAATCTCAGCCGAAAAGAA | ATCGCGAAAAGTGAGGTTTTA |
| | [SEQ ID NO:621] | [SEQ ID NO:622] |
| ORF 2017 | TCTTAGAAGCTGATGCAGTCG | AAAAATTAGCTGTTCCGTTCG |
| | [SEQ ID NO:623] | [SEQ ID NO:624] |
| ORF 3401 | GCTTTTTAATCGCGAGTTTCT | CCAAATCAAAAATAGCGGAGA |
| | [SEQ ID NO:625] | [SEQ ID NO:626] |
| ORF 2074 | CATCGATTTGTTCCTGCTCTA | GGATGTGCCTGAAGAAAGACT |
| | [SEQ ID NO:627] | [SEQ ID NO:628] |
| ORF528-529 | GTGCTGGAAAGATAATGCTGA | AAAAATACCCATTCCAACAGC |
| | [SEQ ID NO:629] | [SEQ ID NO:630] |
| ORF 2815 | GGATAGGGAAATTAGCAGCAC | ATCCCTTCAACTCAAATTCGT |
| | [SEQ ID NO:631] | [SEQ ID NO:632] |
| ORF 2568-2569 | GAACGGTCGGTATTTCATTCT | AACCGTTTGTGCCTCTTTACT |
| | [SEQ ID NO:633] | [SEQ ID NO:634] |
| ORF 2847 | TAAGTGTCGGACTTGTTGTCC | AGCCTTGGCTGATTTAACACT |
| | [SEQ ID NO:635] | [SEQ ID NO:636] |
| ORF1388-1387 | TATTGCCAGTACAGGCTCACT | CGACAAATTTCCAATCAAATG |
| | [SEQ ID NO:637] | [SEQ ID NO:638] |
| ORF 799 | AAATTCCAAAAGGACGTGAGT | CATTGTTTTTCAACCGTTCAT |
| | [SEQ ID NO:639] | [SEQ ID NO:640] |
| ORF 72-73-74 | TGCGTGTATTATGAATCGTCA | GCGAAGTATGCTACTGGGAAT |
| | [SEQ ID NO:641] | [SEQ ID NO:642] |
| ORF1912-1913 | ACCCGGTTAGAACAGCAACTA | AAACTCGCGTATGTATTGTGC |
| | [SEQ ID NO:643] | [SEQ ID NO:644] |
| ORF 150 | TTTTTGCATAAGAAGGTGCAG | TTCAAGAATATCAAAATTCTAACCTTT |
| | [SEQ ID NO:645] | [SEQ ID NO:646] |
| ORF 2777 | AAATCCAAAACCAAAATCTGC | ATGGTATTTGGGATGAGGAAA |
| | [SEQ ID NO:647] | [SEQ ID NO:648] |
| ORF 2759 | TGGATGAAGTGAAAGCAGAAG | CACAACGCTTTAACAACTGGT |
| | [SEQ ID NO:649] | [SEQ ID NO:650] |
| ORF 75 | CCAGGGGCTATAGGTATGAAA | CAACAGTTTCTGGCATTCACT |
| | [SEQ ID NO:651] | [SEQ ID NO:652] |
| ORF 660 | GGTTGCAGGGATACTTTTGTT | TGATCGAGCAGCATCAATAAT |
| | [SEQ ID NO:653] | [SEQ ID NO:654] |
| ORF 2884 | CAGCCCTCTTTTCTGTGTATG | CAAAAAGATTTCGGAACACAA |
| | [SEQ ID NO:655] | [SEQ ID NO:656] |
| ORF 2885 | CATAATCCCCGCAAAAAGTAT | TAATGGATTTGGAATTCATCG |
| | [SEQ ID NO:657] | [SEQ ID NO:658] |
| ORF 1445 | TTCATTTCGATACACCAGGAA | AAAAATGCTCCAAGCTTTCAT |
| | [SEQ ID NO:659] | [SEQ ID NO:660] |
| ORF 1590 | GTGCACTAGCAAATGTGACAA | ACTTAATATTCGGGCTTGCAT |
| | [SEQ ID NO:661] | [SEQ ID NO:662] |
| ORF 182 | CTCTCTGGAAGTTCTCCTTGC | TCTTAGCCAATATCGTGTTGC |
| | [SEQ ID NO:663] | [SEQ ID NO:664] |
| ORF 2017a | ACGCGAAAGATAATTTTGACC | CCTTAGAGCAAATCCCCATAG |
| | [SEQ ID NO:665] | [SEQ ID NO:666] |
| ORF 104-105 | AATGAGACAACGGCTTGGTAT | TACACCCCATCTTCAAATTCA |
| | [SEQ ID NO:667] | [SEQ ID NO:668] |
| ORF 2372 | AGATAGCCTGATGCGAGTTTT | ACGTTTTAGGATCTCCACCTG |
| | [SEQ ID NO:669] | [SEQ ID NO:670] |
| ORF 2110 | AGTGGACAATTGATTGGTGAA | CATCCATCCCTTACTTTGGAC |
| | [SEQ ID NO:671] | [SEQ ID NO:672] |
| ORF 1491 | GGTATTGCTGCAAGTGGTAGA | CCAATCGAACTCGTGTCATAC |
| | [SEQ ID NO:673] | [SEQ ID NO:674] |
| ORF 1490 | GGCACAAAAACTGGTAAGGAA | TGATACAAGTCCTGCAAAAGG |
| | [SEQ ID NO:675] | [SEQ ID NO:676] |
| ORF 1390 | ATGGAGTTGAGGAGGATGAAA | GCTTTTCCCCAGAAGAAAGTT |
| | [SEQ ID NO:677] | [SEQ ID NO:678] |
| ORF 1391 | GCAAGGAAAAATGAAAATGCT | CGCCGGAAAGTTTAGTATGAT |
| | [SEQ ID NO:679] | [SEQ ID NO:680] |
| ORF 1228 | GTAGCTTGGTCCATGAACTTT | TATTGAAATGACGGAAAGTGA |
| | [SEQ ID NO:681] | [SEQ ID NO:682] |
| ORF 29 | CCCAGTGAAAAGTGAAAAAGT | CCCAGTGAAAAGTGAAAAAGT |
| | [SEQ ID NO:683] | [SEQ ID NO:684] |
| ORF 529 | CTGGAAAGCAAATGGAAAATC | CTTTCAATTTCAATGGTCAACA |
| | [SEQ ID NO:685] | [SEQ ID NO:686] |
| ORF 3792 | AAATACTCGGAGGTGCAAAAT | ATCTGGCAACTTTCCATACCT |
| | [SEQ ID NO:687] | [SEQ ID NO:688] |
| ORF 3840 | TCAAATACTTGTCCGCATTTC | GCATATTGATAGTGCGTACCG |
| | [SEQ ID NO:689] | [SEQ ID NO:690] |
| ORF 4101 | AAAAGGAGGCATTTGAATGAA | CGTTGTTTTTCTCCACCAGAT |
| | [SEQ ID NO:691] | [SEQ ID NO:692] |
| ORF 3103 | TGAAGAAGGTTGAAAGCAATG | TTATAACCCGCAGAATTAGGG |
| | [SEQ ID NO:693] | [SEQ ID NO:694] |
| ORF 1490 | TACGCTATTCATTGCGTCTTT | CCAATGGTTATGTTTGGGTTA |
| | [SEQ ID NO:695] | [SEQ ID NO:696] |
| ORF 2217 | CCAACTGTACTGAAGGATTGG | GATGGTTGTAGGTCAGGGTTT |
| | [SEQ ID NO:697] | [SEQ ID NO:698] |
| ORF2969-2968 | CGCTGGAAAGATGGTAACTCTA | GATTAATTCTGTGGCAGAACG |
| | [SEQ ID NO:699] | [SEQ ID NO:700] |
| ORF 4457 | ACAGGGGTAGGTTTAGCACAT | CGTTTTCAGAAGTTTGATCCAT |
| | [SEQ ID NO:701] | [SEQ ID NO:702] |
| ORF 3184 | TACCAATTACGGTGGTTCTCA | CCACATCCAATTGCATAACAT |
| | [SEQ ID NO:703] | [SEQ ID NO:704] |
| ORF 148-149 | AAGAACATCTTCCGCTTTGTT | GATGCAAGAGCCATAACACTC |
| | [SEQ ID NO:705] | [SEQ ID NO:706] |
| ORF 3175 | CAGCAGCAAGAGAGATTTTTG | AATGGATTTCGTTCGTTGTTT |
| | [SEQ ID NO:707] | [SEQ ID NO:708] |
| ORF 2858 | CGGAATATCGGTAGATGCTTT | ATGAATCCTACCACTGCGATA |
| | [SEQ ID NO:709] | [SEQ ID NO:710] |
| ORF 1384-1385 | TTCAAAGGAACAAACATTTCG | GACAGCATGAAAAGGAGATCA |
| | [SEQ ID NO:711] | [SEQ ID NO:712] |
| ORF 2541 | GAGAGCTTACTTTCCCACTCAG | CTTCATCAATACCCGACAAAA |
| | [SEQ ID NO:713] | [SEQ ID NO:714] |
| ORF 2819 | AAATTTTTCAAGGAGGAGCAA | CAATGTCTCTTCTTTCGCTTTT |
| | [SEQ ID NO:715] | [SEQ ID NO:716] |
| ORF 2262 | GTTCAAGATGGACTGCTCAAA | ACACAGGATTAGCACCAACAA |
| | [SEQ ID NO:717] | [SEQ ID NO:718] |
| ORF 817 | ATGCATAGAAAAGAGGGAGCA | CGGCATTAGCAACAGAACTAT |
| | [SEQ ID NO:719] | [SEQ ID NO:720] |
| ORF 757 | CCATTTATTTCCCGCTTTCTA | AACATTCAGACGACGGAACTC |
| | [SEQ ID NO:721] | [SEQ ID NO:722] |
| ORF 2013 | CAAGAAATTCCGTTATGCTGA | GCGACTTCGGTGACTGTATTA |
| | [SEQ ID NO:723] | [SEQ ID NO:724] |
| ORF 2076 | TAACCAATCGTGGAATTTTGA | TGCATTACGAGAAGTGACACA |
| | [SEQ ID NO:725] | [SEQ ID NO:726] |
| ORF 2013 | ACCTGTGTCACACTCAATGGT | ATCATGATTGGAGTGGTCCTT |
| | [SEQ ID NO:727] | [SEQ ID NO:728] |
| L. innocua | | |
| Lin0035 | TGACACAGGAGTTTGTGGTCC | ATGATGGAGAGCAGCAACTATT |
| | [SEQ ID NO:729] | [SEQ ID NO:730] |
| Lin0038 | ACCCTGTACTGCAATGGTCTTT | AATGTTGCCCCTGACTGTAAAC |
| | [SEQ ID NO:731] | [SEQ ID NO:732] |
| Lin0060 | AAGAGAACGAGCAAATGGTATTG | TCAACAAATTACGTAAAATAGCC |
| | [SEQ ID NO:733] | [SEQ ID NO:734] |
| Lin0062 | AATATTGAACAAGCGGAGTGGT | GAATTACAGTCACTGCGACCAA |
| | [SEQ ID NO:735] | [SEQ ID NO:736] |
| Lin0064 | GGGTGGAGCACTTCAAGTTT | TCCCCTTTCTAAAGCTGCTC |
| | [SEQ ID NO:737] | [SEQ ID NO:738] |
| Lin0065 | GTAATGAACAAGATATACAAAAGGG | TTTTCGTTCCTTCTCAATCTTT |
| | [SEQ ID NO:739] | [SEQ ID NO:740] |
| Lin0072 | GCTGTATCACAATTACCTGGCA | TGACAAATCTTTCGAGTTTAACTAT |
| | [SEQ ID NO:741] | [SEQ ID NO:742] |
| Lin0073 | ACGGGCAGAAGAAAAGAGAC | TAAGGAGTCGATTCCCCTTG |
| | [SEQ ID NO:743] | [SEQ ID NO:744] |
| Lin0197 | TCGAGATGCAGAAACCAAAGTA | ATGTAAATCCCACATAAACGGC |
| | [SEQ ID NO:745] | [SEQ ID NO:746] |
| Lin0198 | AACCAAGAAGATGCGATGAAAT | CCGGTAAAGAGTAAACGCAAAT |
| | [SEQ ID NO:747] | [SEQ ID NO:748] |
| Lin0199 | GGAGCAGGAATCATCCAAATAC | CAGAAAATAATTGTTTGCCGAC |
| | [SEQ ID NO:749] | [SEQ ID NO:750] |
| Lin0307 | GCAGAACGAGGCATGTTTTA | CTGAATTGCAGAAGCACCAT |
| | [SEQ ID NO:751] | [SEQ ID NO:752] |
| Lin0308 | GAAAAACCTTGTTTCGGAATTA | CGACTTTGCCAGAAACATACAG |
| | [SEQ ID NO:753] | [SEQ ID NO:754] |
| Lin0332 | CGCAATGACTCCGTTTATCA | TTTGGACAACATGGGTGACT |
| | [SEQ ID NO:755] | [SEQ ID NO:756] |
| Lin0338 | TAATACGCAAGGAGATAATGCGT | TTTGTGCCATTTGATGGAATAC |
| | [SEQ ID NO:757] | [SEQ ID NO:758] |
| Lin0372 | GCAAATGGAAACCAAATTAACAA | GTAATAGCGTCCCTGTTTCGAC |
| | [SEQ ID NO:759] | [SEQ ID NO:760] |
| Lin0397 | ATCCAAAAAGAAAATCAGATGGA | TCAATCCCAAGGAATCTCACTC |
| | [SEQ ID NO:761] | [SEQ ID NO:762] |
| Lin0398 | TGAAATTAGCAATGCAAGGTGT | TCCCTCGCTGGTTTTAGATAGA |
| | [SEQ ID NO:763] | [SEQ ID NO:764] |
| Lin0399 | CCAATATACCCAGCAGTAGCTTG | CGAAATTCCTCTGCATATTTCC |
| | [SEQ ID NO:765] | [SEQ ID NO:766] |
| Lin0415 | TCAATCAGCAAAAGCAGGACTA | GTTGCCGTAACTCCTTTTAACG |
| | [SEQ ID NO:767] | [SEQ ID NO:768] |
| Lin0453 | ACAAATGATTTTCGTGTGGGAT | TAAATTTCAGTCGCGCTGTATC |
| | [SEQ ID NO:769] | [SEQ ID NO:770] |
| Lin0454 | ACGGCTACACAGATGGCTTATT | GAGGATACTTGGGATTCTGTCG |
| | [SEQ ID NO:771] | [SEQ ID NO:772] |
| Lin0455 | GAAGACGAATATTTCGAGGCG | CAAATCTTGTTCTACACTGCCG |
| | [SEQ ID NO:773] | [SEQ ID NO:774] |
| Lin0465 | TCTAGCAGATTGGGAGATAGGC | GCAAACTTTCCATGAGTGCATA |
| | [SEQ ID NO:775] | [SEQ ID NO:776] |
| Lin0476 | CAAAATCATGTTGATTCCTGAT | AAATCTCTTCAAACACATTCGC |
| | [SEQ ID NO:777] | [SEQ ID NO:778] |
| Lin0478 | GGAGAACCACCTTTTGAAGC | CCAAATACCTCGCCAGTCTC |
| | [SEQ ID NO:779] | [SEQ ID NO:780] |
| Lin0559 | AGCGCGTATACAGATACAGCAA | TGGCAATTAAAAGGAGGCTTAC |
| | [SEQ ID NO:781] | [SEQ ID NO:782] |
| Lin0661 | TGGCTAATTTGACGCAATTAAA | TTCACAGTATGCGATGGTAAGC |
| | [SEQ ID NO:783] | [SEQ ID NO:784] |
| Lin0665 | GAAGTAAATGCGCGAACAAA | TGGGATGCCATCTTCATTTA |
| | [SEQ ID NO:785] | [SEQ ID NO:786] |
| Lin0677 | TAGAGAGCGAGGGGAAAAGA | ATGTGTGGAGCAGATGAAGC |
| | [SEQ ID NO:787] | [SEQ ID NO:788] |
| Lin0739 | TAAGCACACTTAGCTCATTGCC | TGTGACCTTCCCACTAAATTCC |
| | [SEQ ID NO:789] | [SEQ ID NO:790] |
| Lin0740 | TAAAGGCGATCCAATCGTAGTT | TGATGGCGCTAGTTCTTTACCT |
| | [SEQ ID NO:791] | [SEQ ID NO:792] |
| Lin0772 | ATCGGAAAAATCCAAGATGC | CGCCCAGATGCAGTTCTAA |
| | [SEQ ID NO:793] | [SEQ ID NO:794] |
| Lin0801 | AAATGGATGGAATCAATGGT | ACGCCATTCCTTTTATTTTT |
| | [SEQ ID NO:795] | [SEQ ID NO:796] |
| Lin0802 | AGTACAGCGATAGGAAGGCAAG | AAGCTCTTGTACCTGCTCAACC |
| | [SEQ ID NO:797] | [SEQ ID NO:798] |
| Lin0803 | GATGAAATACGACGGTGCAA | TCCTTAAAGTCCACCGGATT |
| | [SEQ ID NO:799] | [SEQ ID NO:800] |
| Lin0803 | CCGATAAAGATGGCAATAGA | TCGTATCCACTGTTCCTGTC |
| | [SEQ ID NO:801] | [SEQ ID NO:802] |
| Lin0833 | GAAGAAGAGTGTGGAGGTCGTT | ATCACCAAACATGAAATATGCG |
| | [SEQ ID NO:803] | [SEQ ID NO:804] |
| Lin0834 | AAAGAAGGGGATGCTCTAAAAG | ATGCAAATGTGCTTCAGGATTT |
| | [SEQ ID NO:805] | [SEQ ID NO:806] |
| Lin0835 | TCCAGCTGAGCTAGTAGATTTGAA | CACAATTTATTTTCTCTCCCCG |
| | [SEQ ID NO:807] | [SEQ ID NO:808] |
| Lin0865 | GCGCTACAGAGCAAAGTGTAGA | CGAGTTTCACCACCACCATAAT |
| | [SEQ ID NO:809] | [SEQ ID NO:810] |
| Lin0867 | TTTTAGTTTCCCCAATTGATGC | TTCCTAGCGCTTGTTGTAATCC |
| | [SEQ ID NO:811] | [SEQ ID NO:812] |
| Lin0868 | TTTATCTTGCAAGCCTATGGTGT | ACTCCTGATGGCACATTCATTA |
| | [SEQ ID NO:813] | [SEQ ID NO:814] |
| Lin1065 | TTGCTACACTGCGCTCTTATGT | TGCGTAAAGCTACATTCGAAAA |
| | [SEQ ID NO:815] | [SEQ ID NO:816] |
| Lin1066 | GCTGCACATGAACATTCAGACT | TTAAAGAGGTCCAACCAGGAAC |
| | [SEQ ID NO:817] | [SEQ ID NO:818] |
| Lin1067 | TTGCCTTTTGCTTCCCTTATTA | GTATAAGCGCGTGTACCATCTG |
| | [SEQ ID NO:819] | [SEQ ID NO:820] |
| Lin1068 | AAGGGGAAGAAGCTAAAATTGC | GTACCTTCCAGCGCACTAAATC |
| | [SEQ ID NO:821] | [SEQ ID NO:822] |
| Lin1069 | AATGGGGAAGTCCTCATCTTTT | ATTCCCAGCAATCAACCATATC |
| | [SEQ ID NO:823] | [SEQ ID NO:824] |
| Lin1073 | AGGCGAGAAATATTGGAATACA | AAAACAACTTGGCATGGTACG |
| | [SEQ ID NO:825] | [SEQ ID NO:826] |
| Lin1082 | TTTAATAGAATGTGCCGCAAATAA | TATTACGCTCTGCCATATGTTC |
| | [SEQ ID NO:827] | [SEQ ID NO:828] |
| Lin1083 | TCATTTATGGGCCCCTCTTA | AGGATGGCTCTCAAGGAAAG |
| | [SEQ ID NO:829] | [SEQ ID NO:830] |
| Lin1084 | TCATGCTACAACCCTAGGTGAG | TAACGCACCTCCAAATTCATCT |
| | [SEQ ID NO:831] | [SEQ ID NO:832] |
| Lin1090 | TGAAAACATGGGAGATTCAAA | GGAAAACCATCTTCCCATTC |
| | [SEQ ID NO:833] | [SEQ ID NO:834] |
| Lin1099 | CAATCAAGCAGGTGTTCATCAT | AAAAACTTTCCCAATTTGCTCA |
| | [SEQ ID NO:835] | [SEQ ID NO:836] |
| Lin1204 | AGCAGTTCGATTGGAACGTAAT | CCTTGTGGATAATGAATTTCGG |
| | [SEQ ID NO:837] | [SEQ ID NO:838] |
| Lin1210 | TATGAATCAATTTGCTGGTAAGC | CGAATTCAGCACAATTCTTCTCT |
| | [SEQ ID NO:839] | [SEQ ID NO:840] |
| Lin1211 | GCGAAAGAAATCATTGTTGATG | GCGTGATAATATCCTTGCTCCT |
| | [SEQ ID NO:841] | [SEQ ID NO:842] |
| Lin1212 | AAAGATATCTCCCCGAACTTAGC | CCAAAACCAAAATGCTAAGTAAAA |
| | [SEQ ID NO:843] | [SEQ ID NO:844] |
| Lin1241 | CAGATAAACCCGCACAAATA | TCATATATCGGTGGCATTTC |
| | [SEQ ID NO:845] | [SEQ ID NO:846] |
| Lin1258-57 | TTATGGATTTGTCAGGTGCT | TCAAAGCATGAATCATTTTG |
| | [SEQ ID NO:847] | [SEQ ID NO:848] |
| Lin1258-57 | AAACAGTCGTGAAGTTGCTG | CTTATGTGCGGTCAAGTCAT |
| | [SEQ ID NO:849] | [SEQ ID NO:850] |
| Lin1260 | TGAGGAGGGAACACAATGAA | CACTGCCCACCAACTTTATG |
| | [SEQ ID NO:851] | [SEQ ID NO:852] |
| Lin1262 | GGTCAGCAAGTTTGACAGGA | TTTCTGCGAGCTCTTAATGC |
| | [SEQ ID NO:853] | [SEQ ID NO:854] |
| Lin1298 | GCATTTGAAGGAAGAAATTGAAG | TGCGCTATTGTTATAGTATCGTT |
| | [SEQ ID NO:855] | [SEQ ID NO:856] |
| Lin1299 | AAAAGAATGGGAACTAACCAATGA | AGTACTCAATAATCACTGATGCTGT |
| | [SEQ ID NO:857] | [SEQ ID NO:858] |
| Lin1300 | ATTGGTAATAACGCCGATTACG | CGTATTTTGTTGTCGATCCTGT |
| | [SEQ ID NO:859] | [SEQ ID NO:860] |
| Lin1328 | GCTTCCCAATTTAAAAACCCTT | CGACGTCATATAATCCACCAGA |
| | [SEQ ID NO:861] | [SEQ ID NO:862] |
| Lin1452 | AAAACGAAACCGATATTGTAAA | ACGTTCCAATGAAAGTTACCGT |
| | [SEQ ID NO:863] | [SEQ ID NO:864] |
| Lin1621 | GGATACAGGGGAGAACAAGC | CTGTGACACCTGTGCCTTTT |
| | [SEQ ID NO:865] | [SEQ ID NO:866] |
| Lin1703 | AAAAAGTTAAATCTGCATCAGTGT | TCACCAACAGATACACCATGTTT |
| | [SEQ ID NO:867] | [SEQ ID NO:868] |
| Lin1723 | ATGGTGGTGGTTTGATGAAG | TTTACCGGTGACAATTAAAGGA |
| | [SEQ ID NO:869] | [SEQ ID NO:870] |
| Lin1728 | TAAGGCACCAGTCACTCGAC | CTTTAGCATTTGCTGCATCC |
| | [SEQ ID NO:871] | [SEQ ID NO:872] |
| Lin1731 | GCGATGGCTTTCTAAGTATTGG | TTCCTGACACATTTGTCGATTC |
| | [SEQ ID NO:873] | [SEQ ID NO:874] |
| Lin1744 | AATACAGAAACGGCGAGGAA | TACTGATGCAGCCCAGTTGT |
| | [SEQ ID NO:875] | [SEQ ID NO:876] |
| Lin1745 | GCATAAAAGAGCAAGCACTCAA | TTCCTTAGCTTTAACCTTCGCTT |
| | [SEQ ID NO:877] | [SEQ ID NO:878] |
| Lin1747 | TGGATTACTATCCTGAGCAGTCT | TTTTCCGCGATATTTGTATCCT |
| | [SEQ ID NO:879] | [SEQ ID NO:880] |
| Lin1756 | CAGATAAACCCGCACAAATA | TCATATATCGGTGGCATTTC |
| | [SEQ ID NO:881] | [SEQ ID NO:882] |
| Lin1811 | TTTTAACAATGACGGCTCCA | GCGGGTTTCGAAATGATAGT |
| | [SEQ ID NO:883] | [SEQ ID NO:884] |
| Lin1813 | ATTGATGTTGGAACAAATGC | AAAAATCGCCTGCTTTTAAT |
| | [SEQ ID NO:885] | [SEQ ID NO:886] |
| Lin1898 | TCCCTTGAACAAGAAAACAA | AGCCATACGAAAAGACATCA |
| | [SEQ ID NO:887] | [SEQ ID NO:888] |
| Lin2100 | GGTGAAAAAGTTACTGTGAACC | CGTAAATTTAAGCTACTCACCG |
| | [SEQ ID NO:889] | [SEQ ID NO:890] |
| Lin2373 | GGCCCGTTAGCTTACAAATA | CCTAACGGTGGGGTTAAGG |
| | [SEQ ID NO:891] | [SEQ ID NO:892] |
| Lin2383 | TGATACGAACGCAAGAACAACT | GTAATTTGTCCCAGACACCCAT |
| | [SEQ ID NO:893] | [SEQ ID NO:894] |
| Lin2394 | CACCCAATCATCCAGTGTTAGA | CAGGGTCAATAAAAACAGGCTC |
| | [SEQ ID NO:895] | [SEQ ID NO:896] |
| Lin2394 | CTAAAGACAATCCGGCTCTT | ATGAGCATTGTTGTCTGCTC |
| | [SEQ ID NO:897] | [SEQ ID NO:898] |
| Lin2394 | AGAAGTGAAAGCGACCGAAA | TGGGTCCGGAAACTCAATTA |
| | [SEQ ID NO:899] | [SEQ ID NO:900] |
| Lin2394 | AGAATGGAACAGTGCGGTCT | CCAATTAGCTTGCTGGCTTC |
| | [SEQ ID NO:901] | [SEQ ID NO:902] |
| Lin2397 | ATCGAAGGACGTCATGAACA | TTTCTTCCCCACGATGTAGA |
| | [SEQ ID NO:903] | [SEQ ID NO:904] |
| Lin2405 | CAAGGAGGAACAAGCATGAA | AACAGCATATACGCCCGATT |
| | [SEQ ID NO:905] | [SEQ ID NO:906] |
| Lin2425 | AGGATCCCGGAAAAGGATATTA | TTGTTGTTCCAGCGTTAATTGT |
| | [SEQ ID NO:907] | [SEQ ID NO:908] |
| Lin2494 | GCCCTCCACACAAAAACTTC | GTTTGGGGGAGGGTGAGT |
| | [SEQ ID NO:909] | [SEQ ID NO:910] |
| Lin2603 | TGTTTCCAAATCGGAGCAT | ATTCCAGCTCCCAATTTTTG |
| | [SEQ ID NO:911] | [SEQ ID NO:912] |
| Lin2604 | GCAATCACCTCCAATCAAAA | CCGATTTCGGAACGTAATTT |
| | [SEQ ID NO:913] | [SEQ ID NO:914] |
| Lin2693 | GGCAGCTGGTTTTGCTAACTAT | CCATGAAAATGTTTTTAACCCA |
| | [SEQ ID NO:915] | [SEQ ID NO:916] |
| Lin2724 | CGTTATAGCGCTAAATCTTATACA | CGCTTGAATCAGGCTATCATAA |
| | [SEQ ID NO:917] | [SEQ ID NO:918] |
| Lin2741 | TGACACAATACCACTACCTCGC | CTGTTTTATTCGTCAGAGGGAAA |
| | [SEQ ID NO:919] | [SEQ ID NO:920] |
| Lin2743 | TCGTACAAAAACCAGCATCTTG | GCAAACTACTGTCATGTCGTCC |
| | [SEQ ID NO:921] | [SEQ ID NO:922] |
| Lin2744 | GCAATATGAGCCATGTTGAAAG | GGTGGGTAGCCCATTACACTAA |
| | [SEQ ID NO:923] | [SEQ ID NO:924] |
| Lin2839 | TGGGCTTAGAATGGTCAATAAAA | CTTTTTCATCGTTAACTCGGCT |
| | [SEQ ID NO:925] | [SEQ ID NO:926] |
| Lin2918 | AATTTACCAAGATGCTTTAGGGA | CCCCAAATCAGTTAAAGATAGTTCT |
| | [SEQ ID NO:927] | [SEQ ID NO:928] |
| Lin2941 | CAAGGAATTCAAGGTGTTAAGGA | AGCCAGTTTACAAGAAAGTATTCC |
| | [SEQ ID NO:929] | [SEQ ID NO:930] |
| Lin2945 | AACAAACAGAGAGGACGAGAGC | ATTGAATCCGCTAATTCTTCGT |
| | [SEQ ID NO:931] | [SEQ ID NO:932] |
| L. m. EGDe | | |
| Lmo0017 | TCTTAAAAATGCAGGGGTTGAT | GCCGAATGACCTGAAACATAGT |
| | [SEQ ID NO:933] | [SEQ ID NO:934] |
| Lmo0036 | TTCCGGTTATTAATGGGATGAG | AGCATCAAGCACTTCGTCAGTA |
| | [SEQ ID NO:935] | [SEQ ID NO:936] |
| Lmo0037 | TAATATCGCGGCTTTTCTGTA | TTCCAAAAACAGCTGGTAAA |
| | [SEQ ID NO:937] | [SEQ ID NO:938] |
| Lmo0038 | CGCTTGTAACGACAGAAGAATG | ACACCAACCACTTCACAGTTTG |
| | [SEQ ID NO:939] | [SEQ ID NO:940] |
| Lmo0039 | CTGAAAAGAATCCAGCAATG | CCACCCCTGTTAAAATAACC |
| | [SEQ ID NO:941] | [SEQ ID NO:942] |
| Lmo0040 | AATGAGTAATGATGATGCGTGG | TTCGCTCTCTGTTTGCGTAATA |
| | [SEQ ID NO:943] | [SEQ ID NO:944] |
| Lmo0041 | GCATACAGAGCTAAAACCGTCC | ATTGCAATTGTAGAGATGCGAG |
| | [SEQ ID NO:945] | [SEQ ID NO:946] |
| Lmo0066 | GGAGTCAGTATTTTCTGCCCAC | GCCCTTCACATTCAAATTATCC |
| | [SEQ ID NO:947] | [SEQ ID NO:948] |
| Lmo0067 | ATTGGAAGTGTAACGCGAGAAT | TAGATACCTGCTAGGCTCCCTG |
| | [SEQ ID NO:949] | [SEQ ID NO:950] |
| Lmo0068 | AAGATATGCCATTTTTGAAGGG | TCATTTCTCCCATAATGCACAA |
| | [SEQ ID NO:951] | [SEQ ID NO:952] |
| Lmo0069 | TGTGGGTGTTTCAGGAAGTTTA | CTATTTCGCTTCCACCCTA |
| | [SEQ ID NO:953] | [SEQ ID NO:954] |
| Lmo0070 | TTTGTGACTTGGCTACAAGGTG | TCCCATAGAACACAATGCGATA |
| | [SEQ ID NO:955] | [SEQ ID NO:956] |
| Lmo0071 | CGAGGTAGAAGGAGTCACGTTT | AGGCACATTTCCCACATAATTC |
| | [SEQ ID NO:957] | [SEQ ID NO:958] |
| Lmo0072 | AAACCTTAGAAGTGTATGAGAACGA | CAAAACCTATTATCCCTCCACCT |
| | [SEQ ID NO:959] | [SEQ ID NO:960] |
| Lmo0073 | ACATATTAGATTTCACAATGGGCTT | TATAAGTAAAGGCAGGGCTGGA |
| | [SEQ ID NO:961] | [SEQ ID NO:962] |
| Lmo0074 | TTTACCTTCTTGAAAATGAAATCT | CTTGCAGTATGCAGTGAAAAGG |
| | [SEQ ID NO:963] | [SEQ ID NO:964] |
| Lmo0079 | TCAATAGCCAAACAGGCACTTA | TTTTCCCTTGTTGAACCTCATT |
| | [SEQ ID NO:965] | [SEQ ID NO:966] |
| Lmo0080 | AAAAGGGCTTCAAAGTGATGAA | GTTATCGCTTTAAATGGTGCGT |
| | [SEQ ID NO:967] | [SEQ ID NO:968] |
| Lmo0081 | GCTCAAAGAAGGGACAGAAAAA | GGACTATCTGGAATACCCACACTC |
| | [SEQ ID NO:969] | [SEQ ID NO:970] |
| Lmo0082 | CCAACAGGAGCTAACATGAA | TGGTTAGGAAGATTGGGATT |
| | [SEQ ID NO:971] | [SEQ ID NO:972] |
| Lmo0083 | GAAGGCTTAATAACCCCTGACC | CGCAACCTTCTCCTCATAAATC |
| | [SEQ ID NO:973] | [SEQ ID NO:974] |
| Lmo0084 | CGAAAGATCGAAATGAAATG | CGATGCCAAGTTCTTCTAAA |
| | [SEQ ID NO:975] | [SEQ ID NO:976] |
| Lmo0094 | TGATATTGGTTTATTGATTGGCTT | ACTCGCCACAAACAAACTACAA |
| | [SEQ ID NO:977] | [SEQ ID NO:978] |
| Lmo0106 | AGTTGTCCGGGAGTGGTAGATA | ACCACCAATTGCAATAATTTCC |
| | [SEQ ID NO:979] | [SEQ ID NO:980] |
| Lmo0110 | GCAGCTCCAAAAGATTGTTACC | TTTGAACAATTTCATCTGCCAC |
| | [SEQ ID NO:981] | [SEQ ID NO:982] |
| Lmo0141 | GATCCAATAGAGCTGTTTCCCTT | TTCAGCTATTTCATCTTCGTTATTT |
| | [SEQ ID NO:983] | [SEQ ID NO:984] |
| Lmo0143 | GGATAGGAAAGGTTGGAAGA | TAGGGAGCCCAATTTTTATC |
| | [SEQ ID NO:985] | [SEQ ID NO:986] |
| Lmo0144 | TGGAAGAACAAGAGATCATTGAA | TTCGTAGAATAAGGTTGCACCA |
| | [SEQ ID NO:987] | [SEQ ID NO:988] |
| Lmo0146 | TGAAAGCAGATGGAGAAATGTT | TTGTGCTCTGTTTTACCTGTTGA |
| | [SEQ ID NO:989] | [SEQ ID NO:990] |
| Lmo0147 | GTTTATTTCGAGCGGAAAGT | CATCAGGTTAATTCCGCTTT |
| | [SEQ ID NO:991] | [SEQ ID NO:992] |
| Lmo0151 | TGTTGAATAAAACTAGAGGCGAAA | AGCTACTTTATCTTTAAACTTCGCA |
| | [SEQ ID NO:993] | [SEQ ID NO:994] |
| Lmo0160 | CAGAAACAATTGATGTGGGTGT | CTCCTGCTCCGTTGACTAACTT |
| | [SEQ ID NO:995] | [SEQ ID NO:996] |
| Lmo0171 | AACGTATGAAGCGACGATAA | AAAACCCCACTTGAATTAGC |
| | [SEQ ID NO:997] | [SEQ ID NO:998] |
| Lmo0172 | CCGCATTAATGAAACAAGAA | TGGTAGAATGTTTCGCACTT |
| | [SEQ ID NO:999] | [SEQ ID NO:1000] |
| Lmo0175 | CAAGTGGCATGAACTATACGGA | ATGATTCACAGTGATGTTTGCC |
| | [SEQ ID NO:1001] | [SEQ ID NO:1002] |
| Lmo0200 | TCCAAACCCTACAAAAACAA | GGATTGACAAAATGGAACAA |
| | [SEQ ID NO:1003] | [SEQ ID NO:1004] |
| Lmo0201 | CTGACACAACAAACCTAGCAGC | TGAATTTTGTTTTCACACTCGG |
| | [SEQ ID NO:1005] | [SEQ ID NO:1006] |
| Lmo0202 | ATGCAATTTCGAGCCTAACCTA | GGAGGATTTTCTGCATTCACTC |
| | [SEQ ID NO:1007] | [SEQ ID NO:1008] |
| Lmo0203 | TGAAAAGTTATGTCGGAAACCC | TGAAATTCTTTTTGTCCCCATC |
| | [SEQ ID NO:1009] | [SEQ ID NO:1010] |
| Lmo0204 | GAGGTAAATGCTTCGGACTT | AGGCGATCAATTTCTTCTTC |
| | [SEQ ID NO:1011] | [SEQ ID NO:1012] |
| Lmo0205 | GTATGTGCTTGATCGCAAGTGT | AAAATTATTGGCGTGCATAGGT |
| | [SEQ ID NO:1013] | [SEQ ID NO:1014] |
| Lmo0206 | TTAGTAATCGCGTTATGTTCCG | AGCAACTTTTTGTTCGTCTTCA |
| | [SEQ ID NO:1015] | [SEQ ID NO:1016] |
| Lmo0252 | TTTTGGAAGAGCGGTTCAGTAT | AGGTGATTTCATCCTTTTCCAA |
| | [SEQ ID NO:1017] | [SEQ ID NO:1018] |
| Lmo0253 | CGTAATGGATCAAAGTGGGATT | AAACAGCTTCATCACACGCTAA |
| | [SEQ ID NO:1019] | [SEQ ID NO:1020] |
| Lmo0255 | GCATGTGGAAGTGATTCAAGTG | TTCTTCTTGAACCCTTGAGCTT |
| | [SEQ ID NO:1021] | [SEQ ID NO:1022] |
| Lmo0257 | GTATAGTTGCGCCAATTCATCA | AGATGTCGCACCTTTTCTAAGC |
| | [SEQ ID NO:1023] | [SEQ ID NO:1024] |
| Lmo0262 | TTGGGTGTATCTTCCAATCAGA | CGCTTCTACTATCGGTTGAACA |
| | [SEQ ID NO:1025] | [SEQ ID NO:1026] |
| Lmo0263 | GGGTTACAAAGCATAAAAACGC | ATGTTAAATTTGGGCTTGCGTA |
| | [SEQ ID NO:1027] | [SEQ ID NO:1028] |
| Lmo0264 | CGATGTTTCTCCACTTACTGGTT | AGCAGGCATTTTATCTATCCCA |
| | [SEQ ID NO:1029] | [SEQ ID NO:1030] |
| Lmo0304 | CCTTGGATAGCTGTGCATGATA | CGAATTTCTTGAATGCGTTTCT |
| | [SEQ ID NO:1031] | [SEQ ID NO:1032] |
| Lmo0310 | ATGGAAGAAGTCCTCATTTTGC | CTTCTCTCCCCACACTGAAGTT |
| | [SEQ ID NO:1033] | [SEQ ID NO:1034] |
| Lmo0311 | TACACTTGCCGGAAATAACAAG | CAGCGATTTTATTATTTATTACAGC |
| | [SEQ ID NO:1035] | [SEQ ID NO:1036] |
| Lmo0312 | AGAACTATTTGACGCCTTTCCA | ACAATTTCACCTTTCGTTTCGT |
| | [SEQ ID NO:1037] | [SEQ ID NO:1038] |
| Lmo0313 | TTTCATCCAGATATGGTAGGGG | TTTCAAATCCTGGAATAACTTCCT |
| | [SEQ ID NO:1039] | [SEQ ID NO:1040] |
| Lmo0320 | GGTGTTACAGGTTTGGAGCG | TCAGGAACGGAAACGGGTG |
| | [SEQ ID NO:1041] | [SEQ ID NO:1042] |
| Lmo0333 | AATCCGGTTATTTGGTATGACG | GCAACTGTATAATCTCCGCCTT |
| | [SEQ ID NO:1043] | [SEQ ID NO:1044] |
| Lmo0334 | TGCAACAGAGCTTAGTCAGAAG | CAATCTGCGAATCGTCTTCATA |
| | [SEQ ID NO:1045] | [SEQ ID NO:1046] |
| Lmo0338 | GGTGGAAAGTTTGATTGGTACATAG | GCTTCAATTCATCAGACCTTCTAA |
| | [SEQ ID NO:1047] | [SEQ ID NO:1048] |
| Lmo0378 | TGAGAAAACTTGATTTCAGCTACA | GAATTGCAAACAAATCTCCCTC |
| | [SEQ ID NO:1049] | [SEQ ID NO:1050] |
| Lmo0379 | TCAAAAATAAAGTCTCAAGGACA | GCACAACCATAACTCCGTGATA |
| | [SEQ ID NO:1051] | [SEQ ID NO:1052] |
| Lmo0380 | AAGATGGAACCCTCATTCATAA | ATACATTCCTGCATCTCCATCC |
| | [SEQ ID NO:1053] | [SEQ ID NO:1054] |
| Lmo0381 | TGAACTTCAAAAGAAAATTGATGA | GATGCATCAAAAGAACATTCAA |
| | [SEQ ID NO:1055] | [SEQ ID NO:1056] |
| Lmo0409 | TGCAAAAATTAACCTCGCTACA | CGGTTGTGTTATGAAGCCACTA |
| | [SEQ ID NO:1057] | [SEQ ID NO:1058] |
| Lmo0419 | TTACCGGATGTATTTACCGCAG | GGCGGTTTGTGAGTTGAATAA |
| | [SEQ ID NO:1059] | [SEQ ID NO:1060] |
| Lmo0433 | TAACATCAGTCCCCTAGCAGGT | TCCAATAGTGACAGGTTGGCTA |
| | [SEQ ID NO:1061] | [SEQ ID NO:1062] |
| Lmo0434 | AAAAGCAAGATTTCATGGGAGA | TCTCACTCTTGCATAAGCGGTA |
| | [SEQ ID NO:1063] | [SEQ ID NO:1064] |
| Lmo0440 | TGACAAACAGAACATTAACTACCAA | TTTGAAATCGAATGTTAAATCCA |
| | [SEQ ID NO:1065] | [SEQ ID NO:1066] |
| Lmo0444 | TGAGAATGGTGTCGGTGAATTA | AGCATTTGTGCCACTGTAAATG |
| | [SEQ ID NO:1067] | [SEQ ID NO:1068] |
| Lmo0445 | GGACCTGAGGAACAAGTTAGGA | TTCATACCCATTAATATCCGCC |
| | [SEQ ID NO:1069] | [SEQ ID NO:1070] |
| Lmo0446 | GAAATCTTCTACAGACGGTGCC | AAAACGTTCTGGCGGAGTATAA |
| | [SEQ ID NO:1071] | [SEQ ID NO:1072] |
| Lmo0447 | GTATTGTGGGTATTCTGGGCAT | AGTTTGCGCCATCGTTATAAAT |
| | [SEQ ID NO:1073] | [SEQ ID NO:1074] |
| Lmo0448 | TTCATGCTTGCTTATATGGGTG | TCCGTCCTGGAATTTGATATGT |
| | [SEQ ID NO:1075] | [SEQ ID NO:1076] |
| Lmo0459 | AAGGGCGACTACTCACTTATGC | TAACCGATGATGGATATCACGA |
| | [SEQ ID NO:1077] | [SEQ ID NO:1078] |
| Lmo0460 | TCCAACCTTATGGAGGTTGATT | CATTATCAGAAAACATGGCAGC |
| | [SEQ ID NO:1079] | [SEQ ID NO:1080] |
| Lmo0461 | TTGCTAGTAAGTGTCATTGGAATC | AGACCCGTTTAAAGGTGACTGA |
| | [SEQ ID NO:1081] | [SEQ ID NO:1082] |
| Lmo0462 | GTATGGTGATTTTTAGCGGAGG | TGTCTCCTTGAGGACTTCCAAT |
| | [SEQ ID NO:1083] | [SEQ ID NO:1084] |
| Lmo0463 | ATGTTCCTGTTATCGGCTGAAG | CAACCAAATAAGGACGAGCACT |
| | [SEQ ID NO:1085] | [SEQ ID NO:1086] |
| Lmo0466 | GCAAAAGGAAAAATCCAAGAGA | ACATGCTTTTTCATGAATTGGA |
| | [SEQ ID NO:1087] | [SEQ ID NO:1088] |
| Lmo0467 | GCGAATATTGCACTTGTTGAGA | TACGTCCAAAAGGGTCTACGTT |
| | [SEQ ID NO:1089] | [SEQ ID NO:1090] |
| Lmo0469 | AATAATTGCGGATTTGGAGAAA | GGCCAAGTGAATGACCATAGA |
| | [SEQ ID NO:1091] | [SEQ ID NO:1092] |
| Lmo0470 | CAAACACCGTTTGATAAAGTGC | TGAAGACTCGTAATGACCCACA |
| | [SEQ ID NO:1093] | [SEQ ID NO:1094] |
| Lmo0471 | GGAAGGAAAGGTATTAATCGGG | TTTTTGAAGGATATTGTGCTCG |
| | [SEQ ID NO:1095] | [SEQ ID NO:1096] |
| Lmo0472 | AAAGTGACTAGCGAAGAGGGTG | TGCATATCTGGCTTCGTATTTG |
| | [SEQ ID NO:1097] | [SEQ ID NO:1098] |
| Lmo0473 | TAAAACAGGCACTTACGCTGTG | GCCCTACTTTGGCTAATCTCAA |
| | [SEQ ID NO:1099] | [SEQ ID NO:1100] |
| Lmo0474 | ACATTCGTTTGAGTGTAATCCA | TCAATATCGCTCCATTGACTTG |
| | [SEQ ID NO:1101] | [SEQ ID NO:1102] |
| Lmo0476 | GCATCAAGGTGGTTCGAGTTAT | TTGCTTCAGCCAAAATCTCATA |
| | [SEQ ID NO:1103] | [SEQ ID NO:1104] |
| Lmo0477 | TTCTGCTTAGTGTTATTGGATACG | CACCTTCTGTTTGTCCCGAAC |
| | [SEQ ID NO:1105] | [SEQ ID NO:1106] |
| Lmo0478 | GTAATCCCTAAACATACGGCGA | TCCAATTGTTCTTGTAATGATGG |
| | [SEQ ID NO:1107] | [SEQ ID NO:1108] |
| Lmo0492 | TCATTCAAGCAAAGTCATCCAC | CTGTTTATTAATGGCTGCGACA |
| | [SEQ ID NO:1109] | [SEQ ID NO:1110] |
| Lmo0493 | AGTGGTATATTGCAGCTGAGCA | GTAGGTGGAAAAACTTCGGATG |
| | [SEQ ID NO:1111] | [SEQ ID NO:1112] |
| Lmo0497 | ATCACCGTTTTAGCCGATTATG | TCAGAAGGGATTGCTCTAGTGA |
| | [SEQ ID NO:1113] | [SEQ ID NO:1114] |
| Lmo0514 | ATCTCTATGCGCAAAACTCCAT | ATCATATGTCCCGCTTGAGATT |
| | [SEQ ID NO:1115] | [SEQ ID NO:1116] |
| Lmo0525 | AGGGATTATTATTGGCCGACTT | TGTGTCTGTCCATGTCATCAAA |
| | [SEQ ID NO:1117] | [SEQ ID NO:1118] |
| Lmo0533 | GGGTATTGTCGCAGGTGTTAGT | CTAAAGTTTGTGCATTGCGTTG |
| | [SEQ ID NO:1119] | [SEQ ID NO:1120] |
| Lmo0549 | TTTATCCCTTTTTCCAAAGCTG | TTGTAAGACACAACTTGGGGTG |
| | [SEQ ID NO:1121] | [SEQ ID NO:1122] |
| Lmo0550 | GCTAGTAGTCCAATTAATGTGG | GAATAAAACTAAGCCAATTCCG |
| | [SEQ ID NO:1123] | [SEQ ID NO:1124] |
| Lmo0610 | TTTTATGCCACTCAAATCCCTT | TCTTCGGTAACGGTAGTTTGCT |
| | [SEQ ID NO:1125] | [SEQ ID NO:1126] |
| Lmo0630 | GGCGTGAATAAGTGGTCTAAGC | TCATTTTCGACGCTCTCAAGTA |
| | [SEQ ID NO:1127] | [SEQ ID NO:1128] |
| Lmo0631 | TGCGATTCTACAATTAGTGAATGTT | TAGTGCCACTGTTGCGATGT |
| | [SEQ ID NO:1129] | [SEQ ID NO:1130] |
| Lmo0632 | TTGGAGAGCCAATATCAGGTTT | TCATCTATGTCTTGCCCTTCCT |
| | [SEQ ID NO:1131] | [SEQ ID NO:1132] |
| Lmo0633 | ACGTTTCGGCTTTCTTATTT | TGATTTCTGAAACGCTCATT |
| | [SEQ ID NO:1133] | [SEQ ID NO:1134] |
| Lmo0634 | AGAAGTGGATTTTGATGGGCTA | CATTGGTCGCTGTTATTTCTGA |
| | [SEQ ID NO:1135] | [SEQ ID NO:1136] |
| Lmo0638 | AAAAATTATTTGTGGGGGTTTG | TACCTACACCGAGAGAAGCACA |
| | [SEQ ID NO:1137] | [SEQ ID NO:1138] |
| Lmo0638 | TGCTTGGTTTAACGGATTATCA | GGCTGTTTGTAATTTATTCGTGG |
| | [SEQ ID NO:1139] | [SEQ ID NO:1140] |
| Lmo0673 | CAGCATCCACGAATTATCAAAA | TTCGCTTACAAGGAACCAATTC |
| | [SEQ ID NO:1141] | [SEQ ID NO:1142] |
| Lmo0732 | ATTAGGTATGTCTCTTGCTGG | AGAGTTCGTCGATAAGGTGC |
| | [SEQ ID NO:1143] | [SEQ ID NO:1144] |
| Lmo0733 | GCAAGAAGAAATTGCCATCAA | CCTCCTATAAGGATAATCGGTGC |
| | [SEQ ID NO:1145] | [SEQ ID NO:1146] |
| Lmo0734 | CACTACCAAGGAGCTTTTGA | ATTCAACGGGAACAATCTCT |
| | [SEQ ID NO:1147] | [SEQ ID NO:1148] |
| Lmo0735 | GAAGCGATGTTTTTCATGTTGA | TAGCACAAACCCCTTAACACCT |
| | [SEQ ID NO:1149] | [SEQ ID NO:1150] |
| Lmo0736 | TGGAAATGATCATGTTGGAATC | AAATTCTGCATCCAACCAGTTT |
| | [SEQ ID NO:1151] | [SEQ ID NO:1152] |
| Lmo0737 | ATTATTAGCAGCATTCGGATCG | AATGTCATTGCGAGTGTACCTG |
| | [SEQ ID NO:1153] | [SEQ ID NO:1154] |
| Lmo0738 | ATTCTTAGCGTTTGGACCTC | AAAGCCAACAAATCCTTTTC |
| | [SEQ ID NO:1155] | [SEQ ID NO:1156] |
| Lmo0739 | GGTCGAGTGAAATATTGGGTTC | TTGTTTCACAATATGGGTTTGG |
| | [SEQ ID NO:1157] | [SEQ ID NO:1158] |
| Lmo0745 | AGCTTAATCGTCGTAGGAATCG | ATGCTTCGGAAATTGCATCTAT |
| | [SEQ ID NO:1159] | [SEQ ID NO:1160] |
| Lmo0750 | TTAAAAAGCCGAAAATGAAGGA | AAGCAATAAGCTAAGGCACCAA |
| | [SEQ ID NO:1161] | [SEQ ID NO:1162] |
| Lmo0751 | GATATTGCCAAGTGGTTTGACA | TGGCGCCGTATTACTATAAGTG |
| | [SEQ ID NO:1163] | [SEQ ID NO:1164] |
| Lmo0752 | GTAATGGTAGATCGTCGTGGGT | GGCATTAACATCTTGTGGGAAT |
| | [SEQ ID NO:1165] | [SEQ ID NO:1166] |
| Lmo0753 | AAAATAGCCAATTATGCGAAGG | ATTTTCTTTTTGCATTCTGCCA |
| | [SEQ ID NO:1167] | [SEQ ID NO:1168] |
| Lmo0754 | AAAGGCAGACATCCTTGAGCTA | ATCAATGCCGGGAAGATTATTA |
| | [SEQ ID NO:1169] | [SEQ ID NO:1170] |
| Lmo0780 | GGACTCATGCTAATGCATAAAAGA | TCATCACTCCAAAAATCTTCGTT |
| | [SEQ ID NO:1171] | [SEQ ID NO:1172] |
| Lmo0801 | TTGCGGAAATAACCCTATAAGC | GGGCTATTATACGTTCCATTATCA |
| | [SEQ ID NO:1173] | [SEQ ID NO:1174] |
| Lmo0804 | TTTTACGTTCATCGTTGCCTTA | TGGAAGTTCTTTCTGCCATTTT |
| | [SEQ ID NO:1175] | [SEQ ID NO:1176] |
| Lmo0832 | TGATTTTGGTGTCTCAATAGCA | CAATGCTCGTCTCCATGTTAAA |
| | [SEQ ID NO:1177] | [SEQ ID NO:1178] |
| Lmo0833 | AATAGGGGCTATTCTTTAGCGG | AAAAGCACAGTCATGCGTAGAA |
| | [SEQ ID NO:1179] | [SEQ ID NO:1180] |
| Lmo0834 | TGAATATGACACAAAAAGTGGTGAT | TGAATATGACACAAAAAGTGGTGAT |
| | [SEQ ID NO:1181] | [SEQ ID NO:1182] |
| Lmo0835 | CATTAGCATAGATGGCTTGC | CGAGCTATCACCTGTTTTTG |
| | [SEQ ID NO:1183] | [SEQ ID NO:1184] |
| Lmo0838 | GGAATGCCTCGCATAATATAGG | CAACTGCAATAATCGAGCAAAG |
| | [SEQ ID NO:1185] | [SEQ ID NO:1186] |
| Lmo0842 | AAAAACTGGTCAAGACTTCGGA | TACCATTGCTATCTGTTGTGGC |
| | [SEQ ID NO:1187] | [SEQ ID NO:1188] |
| Lmo0843 | CATGCTTACCGGATACCTGAAT | TACAACCACCAAACCACAACTC |
| | [SEQ ID NO:1189] | [SEQ ID NO:1190] |
| Lmo0849 | AAAGGAAAATGGCGCTTTAATC | CCAGCTTTTTGCAGATCAAGTT |
| | [SEQ ID NO:1191] | [SEQ ID NO:1192] |
| Lmo0880 | AAGCATTTTACATTGGACCGTT | TTTAATTTCTGCAACGGAGACA |
| | [SEQ ID NO:1193] | [SEQ ID NO:1194] |
| Lmo0932 | TTGGCAGGATATGATGAATTTCT | GGTTGGTGAATCAAGTCTACGA |
| | [SEQ ID NO:1195] | [SEQ ID NO:1196] |
| Lmo0933 | TGCTCACGGTAAAGCTGTTATT | CCCAGAATGAAAAGAATTGTCC |
| | [SEQ ID NO:1197] | [SEQ ID NO:1198] |
| Lmo0946 | GATCGGATAGAAGATGGCAAAG | TTGGGGATGTTTCATTATTTCA |
| | [SEQ ID NO:1199] | [SEQ ID NO:1200] |
| Lmo1030 | CCCATTACGAAACACCAATA | ATCGGCTTCTTCTAAACGAG |
| | [SEQ ID NO:1201] | [SEQ ID NO:1202] |
| Lmo1031 | CAGCAAAATTTAAAAAGGCTGG | TTAAGAGCAGCAACCATTCGTA |
| | [SEQ ID NO:1203] | [SEQ ID NO:1204] |
| Lmo1032 | CGGGACATACTGGTTCAGATTT | TCTGCCAAAGGTTCTACACTCA |
| | [SEQ ID NO:1205] | [SEQ ID NO:1206] |
| Lmo1033 | GACGTGGCTTATTCAGACACAA | GCGAACTACTTACCACTTCGCT |
| | [SEQ ID NO:1207] | [SEQ ID NO:1208] |
| Lmo1034 | ATGCTTGGTTGTTATTTTCGCT | TGTCCAGGAATTAGCACAACAC |
| | [SEQ ID NO:1209] | [SEQ ID NO:1210] |
| Lmo1035 | TTAATTGTGGTGCCTGTGTCTC | AAAATCCCAGCCTATTCCATCT |
| | [SEQ ID NO:1211] | [SEQ ID NO:1212] |
| Lmo1036 | AGAAATGGCAGTGAATCAAGGT | CTTTTCGTACATCTTCTCGGCT |
| | [SEQ ID NO:1213] | [SEQ ID NO:1214] |
| Lmo1060 | TAACCGAAGCAGGGTATCAAGT | TTGCGTAGATTTTTCATATGCG |
| | [SEQ ID NO:1215] | [SEQ ID NO:1216] |
| Lmo1061 | ATTAAAATCCCCTCTTGCTG | TGATTTCTCAAGGCGATAGA |
| | [SEQ ID NO:1217] | [SEQ ID NO:1218] |
| Lmo1062 | AAACCCCAAGTTTTTCAGTCAA | CATTTCAAATTTACGCTCACGA |
| | [SEQ ID NO:1219] | [SEQ ID NO:1220] |
| Lmo1063 | GGGAAACAAAGCTCAAATTGTT | AATAATGTGTTCCGAGGTTTCA |
| | [SEQ ID NO:1221] | [SEQ ID NO:1222] |
| Lmo1076 | AACTTTGCCAAATACCCATCAC | ACCTGGAACCTCAGACCATAAA |
| | [SEQ ID NO:1223] | [SEQ ID NO:1224] |
| Lmo1077 | TGGTGGTAATTTTGAGTTCG | TTGTTTGCCTTGAGTTTCTG |
| | [SEQ ID NO:1225] | [SEQ ID NO:1226] |
| Lmo1079 | CTGGTTATTGGGCAAACTTAGG | TCTTTTTATCTGCTCCCCCATA |
| | [SEQ ID NO:1227] | [SEQ ID NO:1228] |
| Lmo1080 | AATTCCAGTAGTTTCGGACGTG | TTGGCTCATACGTAAATTCAGC |
| | [SEQ ID NO:1229] | [SEQ ID NO:1230] |
| Lmo1081 | AAGAAAAACCGGAAGGATTAGC | CAGCCTCATCAATATACCCCAT |
| | [SEQ ID NO:1231] | [SEQ ID NO:1232] |
| Lmo1082 | TGGAAATTGTTGAAAGTAAACTAGC | TGGAAATTGTTGAAAGTAAACTAGC |
| | [SEQ ID NO:1233] | [SEQ ID NO:1234] |
| Lmo1083 | TTAACTTCGCAGCAGAATCTCA | TCGTACGTTCATTATGTCCACC |
| | [SEQ ID NO:1235] | [SEQ ID NO:1236] |
| Lmo1084 | TGTGCCGCTTTTACTAATGT | GTTGCAAACTCAAACCATGT |
| | [SEQ ID NO:1237] | [SEQ ID NO:1238] |
| Lmo1085 | GCTAGCTATTCGCTCTCTTTGG | GGTAAATCGGCATGAAAGTAGC |
| | [SEQ ID NO:1239] | [SEQ ID NO:1240] |
| Lmo1088 | ACCTCGAACGGATGTTTATT | CCTTCGTTTTTATTTGCTCA |
| | [SEQ ID NO:1241] | [SEQ ID NO:1242] |
| Lmo1090 | TGATGGAGCGACTGATAATTCA | GGTACGCAAGTTGATCTTTTGA |
| | [SEQ ID NO:1243] | [SEQ ID NO:1244] |
| Lmo1091 | TGGTAGGTGCTACCCTTACA | TGATGAATAAAACCCCACAA |
| | [SEQ ID NO:1245] | [SEQ ID NO:1246] |
| Lmo1097 | ACCTAAGGTGGAGTTCTGGACA | CATGTCCTAGGCGCTCTTTAAT |
| | [SEQ ID NO:1247] | [SEQ ID NO:1248] |
| Lmo1099 | TCTACGATGAGAACAAACGACG | TCAATGTTCCTCCATGAGTTTT |
| | [SEQ ID NO:1249] | [SEQ ID NO:1250] |
| Lmo1101 | AGGTGCTACCCTTACAGACGAG | TCGATAATGATGAATAAAACCCC |
| | [SEQ ID NO:1251] | [SEQ ID NO:1252] |
| Lmo1102 | TGAATGACATTTGTGAAATAACCTG | TTTTGAACTACTCCAGCTTTACTTA |
| | [SEQ ID NO:1253] | [SEQ ID NO:1254] |
| Lmo1103 | AACAGTCGCAAGATTCACAGAG | GTCTGCTTGTGTGTAAACAGGG |
| | [SEQ ID NO:1255] | [SEQ ID NO:1256] |
| Lmo1104 | CAGCAGAGGTACTGGCACATAG | GTGTCAATCCGCTACAATCAAA |
| | [SEQ ID NO:1257] | [SEQ ID NO:1258] |
| Lmo1105 | AGACACCAACAACGGAGAAGAT | ATTTTTACCAATTGCTCGGCTA |
| | [SEQ ID NO:1259] | [SEQ ID NO:1260] |
| Lmo1106 | GCGTTGTGATGAAGTCAAAGAC | CCTTTTTACGGCTCAAAATGAC |
| | [SEQ ID NO:1261] | [SEQ ID NO:1262] |
| Lmo1107 | CTACACCAGCATTTGGAACGTA | CTTTTGAAGTTTCACGGCTTTT |
| | [SEQ ID NO:1263] | [SEQ ID NO:1264] |
| Lmo1108 | ACCCCACCTATCGACTATGAAG | TCAAATACCCCATGTGAGGTTAC |
| | [SEQ ID NO:1265] | [SEQ ID NO:1266] |
| Lmo1110 | GGCTTAACGGCTGAACTCTATG | TTCACTCATGTATTTCTTGCGG |
| | [SEQ ID NO:1267] | [SEQ ID NO:1268] |
| Lmo1111 | AACACCGAAGCTGGTATGACTT | TTCTTACCAATGAACCATTCCC |
| | [SEQ ID NO:1269] | [SEQ ID NO:1270] |
| Lmo1112 | TCAAAGACAAAAGAGCGAATCA | TTCACTTCGTGCAATCATATCC |
| | [SEQ ID NO:1271] | [SEQ ID NO:1272] |
| Lmo1113 | GAGATTAGCCGAAGGAATCGTA | TGCTGGTCTTTTCAAGACGATA |
| | [SEQ ID NO:1273] | [SEQ ID NO:1274] |
| Lmo1114 | CACGTAATACCCAACATGGAAA | TGTGAATCCACGAGTACCAATC |
| | [SEQ ID NO:1275] | [SEQ ID NO:1276] |
| Lmo1115 | TAAACTGCTTGGAGAAACCA | ATGTCTTGTCGTGATCCTTG |
| | [SEQ ID NO:1277] | [SEQ ID NO:1278] |
| Lmo1115 | GGACAATGGCACTCTCCTAACT | TTTAAGCATAGTCTCGTTGCCA |
| | [SEQ ID NO:1279] | [SEQ ID NO:1280] |
| Lmo1116 | TGCTGCTCACTTTGGTTATGAT | TGATTGCCAGAACATTTAGGAA |
| | [SEQ ID NO:1281] | [SEQ ID NO:1282] |
| Lmo1117 | TGGCTTTAAATGCAAAGAGTATCA | GGTACATCTTCTATCACCTCGTCA |
| | [SEQ ID NO:1283] | [SEQ ID NO:1284] |
| Lmo1118 | AGGGGTCTTAAATCCTGGAATA | GAAGTTTTCCGCTCCCTATTTT |
| | [SEQ ID NO:1285] | [SEQ ID NO:1286] |
| Lmo1119 | GGTTCTGGTCTTGCCTTAGAACT | TCTCTGGCGCTAAATCATAAGT |
| | [SEQ ID NO:1287] | [SEQ ID NO:1288] |
| Lmo1120 | AAAACGAGTCCATGGGAGACTA | TCATTTCTTCAACTTCATTGTGC |
| | [SEQ ID NO:1289] | [SEQ ID NO:1290] |
| Lmo1121 | GCATGAAATTAGACGATAATGCAA | ACTTTCTTGGCAAAATTGTAAGT |
| | [SEQ ID NO:1291] | [SEQ ID NO:1292] |
| Lmo1125 | TCCATCCTATTTGAAAAGCACA | CTAGAAAATCAAAACCGTCCGT |
| | [SEQ ID NO:1293] | [SEQ ID NO:1294] |
| Lmo1133 | TCAAACTACCGGCAAAGTTTCT | AAATTCAACCGTGTCAAGTTCC |
| | [SEQ ID NO:1295] | [SEQ ID NO:1296] |
| Lmo1134 | AAATTTCCCACAAAGCAAAT | GTATCCGATTTGGGAGTGTT |
| | [SEQ ID NO:1297] | [SEQ ID NO:1298] |
| Lmo1135 | AGTTCCGCTTGTTATTCGTA | CAAACGATATCTCCCCATCT |
| | [SEQ ID NO:1299] | [SEQ ID NO:1300] |
| Lmo1136 | AGTCATCCAGATGCCAAGAAAT | ATGGATTGTCTATCTCAACCGC |
| | [SEQ ID NO:1301] | [SEQ ID NO:1302] |
| Lmo1188 | AGGTTAGAGGTTGCACGAATGT | ATTCGGACAAAAATTTGAATGG |
| | [SEQ ID NO:1303] | [SEQ ID NO:1304] |
| Lmo1247 | ATGGAAAGGCCAGATAATCAAC | AATCTCCTTGTTTGGTCATGCT |
| | [SEQ ID NO:1305] | [SEQ ID NO:1306] |
| Lmo1263 | TGGCAAAAATAACAGGAAGTGT | CACGAGCAACAAGCACATACTC |
| | [SEQ ID NO:1307] | [SEQ ID NO:1308] |
| Lmo1289 | AGGTCACTGGTAATCGTCCC | TCAGACTGTCAAATTACTAACTTC |
| | [SEQ ID NO:1309] | [SEQ ID NO:1310] |
| Lmo1302 | TCGCATCCAGTTCTACTGTT | TAAAGCCCGATTACTTTTCC |
| | [SEQ ID NO:1311] | [SEQ ID NO:1312] |
| Lmo1303 | GATGCCCATGAACAGTAGAA | AGCTAACAAAGTCCGCTTTC |
| | [SEQ ID NO:1313] | [SEQ ID NO:1314] |
| Lmo1307 | ATCTTTCTTCCACGGATAGC | TTGTTCAAAAAGGGCTGTTA |
| | [SEQ ID NO:1315] | [SEQ ID NO:1316] |
| Lmo1413 | CACTTGCAACATCCGATACATT | TCACTCGTGAAAACGCTACTGT |
| | [SEQ ID NO:1317] | [SEQ ID NO:1318] |
| Lmo1441 | ATGTCGAAGCAACAGTAGCAGA | AATTCCGAGATGCAGAGGATAA |
| | [SEQ ID NO:1319] | [SEQ ID NO:1320] |
| Lmo1451 | AGTAGATGGCGCTAATCGAGAA | CTTGGGCTAAACGAGCAGTATT |
| | [SEQ ID NO:1321] | [SEQ ID NO:1322] |
| Lmo1478 | TTTGTCGATTTATACATTGCGTT | TGACATAATTTCTCTCGGCAGT |
| | [SEQ ID NO:1323] | [SEQ ID NO:1324] |
| Lmo1597 | GGTGGTGGTGAGTTGAATCTTA | TGCAGTCACCTTAGCACCATTA |
| | [SEQ ID NO:1325] | [SEQ ID NO:1326] |
| Lmo1604 | GGCAGAACGTTTAGTAGGCAC | ACCGGGTTGCCAGTTAATC |
| | [SEQ ID NO:1327] | [SEQ ID NO:1328] |
| Lmo1648 | GGTTTTGATCCTTGGGATAGTG | TTTCAGGATTATTTCCCCACAT |
| | [SEQ ID NO:1329] | [SEQ ID NO:1330] |
| Lmo1656 | GACAAGAATCGGACATTTCA | CATCGCTTTTTCAGCTTTTA |
| | [SEQ ID NO:1331] | [SEQ ID NO:1332] |
| Lmo1666 | TGTAGTTGATGTTGGAAGTCCG | TATAATCGGCGCTACCGTATCT |
| | [SEQ ID NO:1333] | [SEQ ID NO:1334] |
| Lmo1700 | GTCGTGCCATTATTATTCGCTT | TCCAGTGCCAATTACCAATATG |
| | [SEQ ID NO:1335] | [SEQ ID NO:1336] |
| Lmo1707 | ATGAATCTTGAAACCCCTTCG | TGGATTTGCGTTTAATTAGCTG |
| | [SEQ ID NO:1337] | [SEQ ID NO:1338] |
| Lmo1714 | CGTGCATTACGGTAAAGTTGGT | CCTTCTTCTTCGCTGAAAAT |
| | [SEQ ID NO:1339] | [SEQ ID NO:1340] |
| Lmo1786 | GGGAAGCAAAGTGTTACAGACC | GATATCCATCTTCCATCTGGGT |
| | [SEQ ID NO:1341] | [SEQ ID NO:1342] |
| Lmo1799 | ACAACAACGATGATGGTAGC | CATCATTGGCACTAGGTAGC |
| | [SEQ ID NO:1343] | [SEQ ID NO:1344] |
| Lmo1968 | GTGCAATCAGCCAACAAAATAA | TTCAAATTCACAGGCATGAATC |
| | [SEQ ID NO:1345] | [SEQ ID NO:1346] |
| Lmo1969 | ATGGCAATTATTCGGGATGTAG | TTGATTGAGCATAGCCTTTGTG |
| | [SEQ ID NO:1347] | [SEQ ID NO:1348] |
| Lmo1970 | AGTAACACGAGCTGTTGCAT | AAATCACACGCACTCCTTTA |
| | [SEQ ID NO:1349] | [SEQ ID NO:1350] |
| Lmo1971 | ACAGGAAATGATGAAGTCGGTT | AATAATGGCTCCTCGTCTACCA |
| | [SEQ ID NO:1351] | [SEQ ID NO:1352] |
| Lmo1973 | GGAGGTAGAAAGTCCGGTAG | TCCAACTGTAAAGCCATGTT |
| | [SEQ ID NO:1353] | [SEQ ID NO:1354] |
| Lmo1974 | TGACAGTTCGAAAAGCGATAGA | ATAGGAAGTACGTCGCACATGA |
| | [SEQ ID NO:1355] | [SEQ ID NO:1356] |
| Lmo2026 | TATGATGATGGGACTTCTGC | ATTTGCTTTCATCCAATGTG |
| | [SEQ ID NO:1357] | [SEQ ID NO:1358] |
| Lmo2027 | TGGTGGAGATCTAAAAGCAA | TGGCACATCATCACTTGTAA |
| | [SEQ ID NO:1359] | [SEQ ID NO:1360] |
| Lmo2045 | CCAGTTGTGGTATTTATTTTGACG | AAAGCGCCACATACAATAAAGC |
| | [SEQ ID NO:1361] | [SEQ ID NO:1362] |
| Lmo2067 | TACCGCTGTCTCCATTACATTG | GTTGCTCATTGACGAGTGGATA |
| | [SEQ ID NO:1363] | [SEQ ID NO:1364] |
| Lmo2085 | TTTAATGGGGATGGTATTCAGC | ATTGATCCTGTTTTCTCAGCGT |
| | [SEQ ID NO:1365] | [SEQ ID NO:1366] |
| Lmo2143 | GTAGTGTCGCTGGCTTTCTCTT | AGTTGCTCCGTTGAAACTCAAT |
| | [SEQ ID NO:1367] | [SEQ ID NO:1368] |
| Lmo2144 | GGAAAACCGGAGATAAAATTCC | ATTGGTTCCTGCTTATCGATTG |
| | [SEQ ID NO:1369] | [SEQ ID NO:1370] |
| Lmo2157 | GGAAAACCGGAGATAAAATTCC | ATTGGTTCCTGCTTATCGATTG |
| | [SEQ ID NO:1371] | [SEQ ID NO:1372] |
| Lmo2178 | GACGCCAGCTGGTTTTTATATC | ATACGCGTGAATTCTCCAAGTT |
| | [SEQ ID NO:1373] | [SEQ ID NO:1374] |
| Lmo2197 | AAAAATCAGACTGTTAGTTTAAGCA | GTCAACATAACAAAAATGGAACT |
| | [SEQ ID NO:1375] | [SEQ ID NO:1376] |
| Lmo2276 | TGTTCGTTCTGAAAATGGGAAT | TTATCTTCCGCATCTCTACGTG |
| | [SEQ ID NO:1377] | [SEQ ID NO:1378] |
| Lmo2277 | TGAAGCATCAGCGGAATATAGA | GAGAGAATGTATGGCTCTTTGG |
| | [SEQ ID NO:1379] | [SEQ ID NO:1380] |
| Lmo2278 | TCAAACAATGCGCTCTTATCAT | ACCGGTTTTATGCTTTTCAGTG |
| | [SEQ ID NO:1381] | [SEQ ID NO:1382] |
| Lmo2282 | CAGTACCGCAAATAGTACGG | AAAGCCTCTTGGGTGAGTAG |
| | [SEQ ID NO:1383] | [SEQ ID NO:1384] |
| Lmo2283 | TGAAAAATGGGATGATTACG | TATTCAGTGACCACGGTTCT |
| | [SEQ ID NO:1385] | [SEQ ID NO:1386] |
| Lmo2296 | ACACCAGAGGTTTTTAATGCGT | GTCGGGATATTAACTTCTCCCC |
| | [SEQ ID NO:1387] | [SEQ ID NO:1388] |
| Lmo2301 | TTCAAAACGTATGCAGGATGAC | GTTGCAAAGCCAGAATCCTTAC |
| | [SEQ ID NO:1389] | [SEQ ID NO:1390] |
| Lmo2302 | GCTGACTGGGTAAGTGGGTTAG | TCACAGGCCCTTTATTTTTCTT |
| | [SEQ ID NO:1391] | [SEQ ID NO:1392] |
| Lmo2311 | GTGCTTTTGAACGATTTGACAC | TCAATTTCTTCCACCATTTCAA |
| | [SEQ ID NO:1393] | [SEQ ID NO:1394] |
| Lmo2312 | GTTGTGGTAATGCGAAATGT | AAATCTGCACCCCTTAAATC |
| | [SEQ ID NO:1395] | [SEQ ID NO:1396] |
| Lmo2313 | TATTGGCAGTTTGCTGTTCT | CCGCAAATGAATCTGTGTAT |
| | [SEQ ID NO:1397] | [SEQ ID NO:1398] |
| Lmo2318 | AAAAATCTGCGGCTAATCCTTT | GCTTTCTAGTACAAACGTGCCA |
| | [SEQ ID NO:1399] | [SEQ ID NO:1400] |
| Lmo2319 | AATGGCGGAGGAATTAGACAC | CTTCCTTCTTTTAGAGCGCTTG |
| | [SEQ ID NO:1401] | [SEQ ID NO:1402] |
| Lmo2322 | ACGAAGGTTACGAGTCGAAGAG | ATAGTGTGCGCCTCGGTATAAT |
| | [SEQ ID NO:1403] | [SEQ ID NO:1404] |
| Lmo2324 | ACTTTGAGTAAAACCAATGGGC | GTGGTTTTCATTAAGCCGTTTC |
| | [SEQ ID NO:1405] | [SEQ ID NO:1406] |
| Lmo2331 | TGTGGCGGAACAGATAATACAA | AATTTCGTCATACGTTTCACTGTT |
| | [SEQ ID NO:1407] | [SEQ ID NO:1408] |
| Lmo2387 | GCGGCTTTAATTGGTATCGTAG | GGGAATCGAATAATTTTGGACA |
| | [SEQ ID NO:1409] | [SEQ ID NO:1410] |
| Lmo2408 | TCCTTTGCTAAGTTGTTGAGA | TTCAATGGCATGAATTGTTT |
| | [SEQ ID NO:1411] | [SEQ ID NO:1412] |
| Lmo2409 | TTTCAATGATTGCTGCTATTC | GAAAAATGGTAAACCAGGAAA |
| | [SEQ ID NO:1413] | [SEQ ID NO:1414] |
| Lmo2410 | ATTTGCCTCACTCCTTTATCCA | TTTCCTGGTTTACCATTTTTCG |
| | [SEQ ID NO:1415] | [SEQ ID NO:1416] |
| Lmo2443 | TTATTCCCTTGGATGTCTAGCG | ATAAAATACTTGCTGACACCGC |
| | [SEQ ID NO:1417] | [SEQ ID NO:1418] |
| Lmo2451 | AGGTAAAAGTGCTGGCTTATCC | CTGTACAAAGTATGCCAGTGCAA |
| | [SEQ ID NO:1419] | [SEQ ID NO:1420] |
| Lmo2548 | GAATTCATCCTGAGTACCGTCC | GCAGTCGCATGTTTTTGTTTAC |
| | [SEQ ID NO:1421] | [SEQ ID NO:1422] |
| Lmo2567 | ATCGCACTTAATATCGTACTTGATA | GGAGAATTACCCCACATACGAT |
| | [SEQ ID NO:1423] | [SEQ ID NO:1424] |
| Lmo2576 | TTAATACCGCCGGAAAATTATG | GTTTCTGTATCCGAGGTCAAGG |
| | [SEQ ID NO:1425] | [SEQ ID NO:1426] |
| Lmo2594 | TTTGTAACAGCATGCTCAACCT | TATTTGGGCTGGTGAAGTTTCT |
| | [SEQ ID NO:1427] | [SEQ ID NO:1428] |
| Lmo2595 | ACGGTGAAGCAACTATAGAGGG | CGTACTTTCGTCGCCTAATTGT |
| | [SEQ ID NO:1429] | [SEQ ID NO:1430] |
| Lmo2671 | CAAATATTTCCACACAAGATCCG | CACTCAGCCACACAAGGTTCT |
| | [SEQ ID NO:1431] | [SEQ ID NO:1432] |
| Lmo2686 | TAGCGCTTCCGAAATAGAGAAT | CCCGCTTACTGATGCTTTTAAC |
| | [SEQ ID NO:1433] | [SEQ ID NO:1434] |
| Lmo2731 | GGAATGGAGTATCAATTCGGAG | TTTTGCGAAGTTGTGATAGTGG |
| | [SEQ ID NO:1435] | [SEQ ID NO:1436] |
| Lmo2732 | TAAAGCTTTCCGGTTTCTCTGA | GGGCATGATGATGTTTAAATGA |
| | [SEQ ID NO:1437] | [SEQ ID NO:1438] |
| Lmo2733 | GAATGCCTTTTATCCAAAGACG | AAATCCGGCAATGATACCTAAA |
| | [SEQ ID NO:1439] | [SEQ ID NO:1440] |
| Lmo2734 | AACAAGCGCACTTAGAATGACA | CATTTTCGAAGCGAATTAGGTC |
| | [SEQ ID NO:1441] | [SEQ ID NO:1442] |
| Lmo2735 | AACATCTGGCACGACTTGTATG | TAGTAAATCGCTGGGACACCTT |
| | [SEQ ID NO:1443] | [SEQ ID NO:1444] |
| Lmo2736 | GCCAACACGATTGGATTAGATT | TCGGCGTCTTTCATTTTATCTT |
| | [SEQ ID NO:1445] | [SEQ ID NO:1446] |
| Lmo2771 | ATTAACAAATGAGGTGAACGGC | CGAATTTTTATAGGCAGGATCG |
| | [SEQ ID NO:1447] | [SEQ ID NO:1448] |
| Lmo2772 | TTGACGCTTGTTGTTATTGGAC | CATTTGCTCATCAATACCGAAA |
| | [SEQ ID NO:1449] | [SEQ ID NO:1450] |
| Lmo2773 | AAGGGGCTTGGCTTTAATTCTA | CCAATAATTACGCGTTGACAGA |
| | [SEQ ID NO:1451] | [SEQ ID NO:1452] |
| Lmo2776 | TAGCGATAGCATTGCTTACAGTG | GCCCAATTAGCTTTATTACCCC |
| | [SEQ ID NO:1453] | [SEQ ID NO:1454] |
| Lmo2781 | GAACCGAAGTCTGATTGGAGAC | AATTACAGGCTTTGCAGTACGC |
| | [SEQ ID NO:1455] | [SEQ ID NO:1456] |
| Lmo2782 | GCTGATGTGTAATGCAGGAATG | CATACGGCCATAATCAACGATA |
| | [SEQ ID NO:1457] | [SEQ ID NO:1458] |
| Lmo2786 | TGAAAACAAGGGTTTTTGGAGT | TGCTTCGTAAGCTAATTCCCAT |
| | [SEQ ID NO:1459] | [SEQ ID NO:1460] |
| Lmo2787 | TTTTAGGCTACACGAGTGCAAA | CTTGCTGTAAATGACATTGGGA |
| | [SEQ ID NO:1461] | [SEQ ID NO:1462] |
| Lmo2807 | GGAGTAAAATTCTATATGGATGAGA | AGATACTTGCTCCTCCGCAC |
| | [SEQ ID NO:1463] | [SEQ ID NO:1464] |
| Lmo2809 | TAATGATTGTTGCGGGATTAGG | TTGTAAGACACAACTTGGGGTG |
| | [SEQ ID NO:1465] | [SEQ ID NO:1466] |
| Lmo2821 | TGACGTCTTCTCCTTTGATGG | TAGACTGCCAAGCCGCTGG |
| | [SEQ ID NO:1467] | [SEQ ID NO:1468] |
| rpoB (a) | GCGGATGAAGAGGATAATTACG | GGAATCCATAGATGGACCGTTA |
| | [SEQ ID NO:1469] | [SEQ ID NO:1470] |
| rpoC (d) | AATCCGTCTTCACCCTCTTGTA | CGAGGATTTTCTTCTTGAATGG |
| | [SEQ ID NO:1471] | [SEQ ID NO:1472] |
| dnaN (a) | TTTCCATCGAAGCATTTATTCC | ATTTCAATAGATTCGCTTGCGT |
| | [SEQ ID NO:1473] | [SEQ ID NO:1474] |
| gyrB (a) | ATTCGTTCTTACGTGGAGCATT | GGATTACGGGAAGAACAGTCAG |
| | [SEQ ID NO:1475] | [SEQ ID NO:1476] |

TABLE 8
Probes spotted on the membrane and primary hybridization datXa

| | | |
|---|---|---|
| Gene | Description | Functional category |

L. monocytogenes EGDe genes

| | | |
|---|---|---|
| Lmo0017 | 372aa/Unknown, similar to Bacillus anthracis CapA protein | Cell wall |
| | (polyglutamate capsule biosynthesis) | |
| Lmo0036 | 341aa/Unknown, similar to ornithine carbamoyltransferase | Metabolism of amino acids and related molecules |
| Lmo0037 | 461aa/Unknown, similar to amino acid transporter | Transport/binding proteins and lipoproteins |
| Lmo0038 | 363aa/Unknown, conserved hypothetical protein | unknown |
| Lmo0039 | 313aa/carbamate kinase | Metabolism of amino acids and related molecules |
| Lmo0040 | 369aa/Unknown, conserved hypothetical protein | Phage-related functions |
| Lmo0041 | 259aa/Unknown, conserved hypothetical protein, | Regulation |
| | hypothetical regulator | |
| Lmo0066 | 604aa/Unknwon, similar to toxin components | miscellaneous |
| Lmo0067 | 329aa/Unknown, similar to dinitrogenase reductase ADP- | Protein modification |
| | ribosylation system | |
| Lmo0068 | 107aa/Unknwon | unknown |
| Lmo0069 | 97aa/Unknwon | unknown |
| Lmo0071 | 85aa/Unknwon | unknown |
| Lmo0072 | 67aa/Unknown, Hypothetical | unknown |
| Lmo0073 | 94aa/Unknwon | unknown |
| Lmo0074 | 149aa/Unknwon | unknown |
| Lmo0079 | 492aa/unknown | unknown |
| Lmo0080 | 132aa/Unknwon | unknown |
| Lmo0081 | 200aa/Unknwon | unknown |
| Lmo0082 | 108aa/Unknwon | unknown |
| Lmo0083 | 122aa/Unknown, similar to transcription regulator (merR | Regulation |
| | family) | |
| Lmo0084 | 327aa/Unknwon, similar to oxidoreductases | Specific pathways |
| Lmo0094 | 186aa/Unknwon | unknown |
| Lmo0106 | 300aa/Unknown, similar to transcription regulator | Regulation |
| Lmo0110 | 371aa/Unknown, similar to lipase | Metabolism of lipids |
| Lmo0141 | 80aa/Unknwon | unknown |
| Lmo0143 | 58aa/Unknwon | unknown |
| Lmo0144 | 147aa/Unknwon | unknown |
| Lmo0146 | 68aa/Unknwon, hypothetical protein | unknown |
| Lmo0147 | 145aa/Unknwon | unknown |
| Lmo0151 | 135aa/Unknwon | unknown |
| Lmo0160 | 571aa/Unknown, putative peptidoglycan bound protein | Cell surface proteins |
| | (LPXTG motif) | |
| Lmo0171 | 832aa/Unknwon, similar to internalin proteins, putative | cell surface proteins |
| | peptidoglycan bound protein (LPXTG motif) | |
| Lmo0172 | 112aa/Unknown, similar to transposase C-terminal part | Transposon and IS |
| Lmo0175 | 415aa/Unknown, putative peptidoglycan bound protein | cell surface proteins |
| | (LPXTG motif) | |
| Lmo0200 | 237aa/prfA: listeriolysin positive regulatory protein | Regulation |
| Lmo0201 | 317aa/plcA: phosphatidylinositol-specific phospholipase c | virulence |
| Lmo0202 | 529aa/hly | virulence |
| Lmo0203 | 510aa/mpl: Zinc metalloproteinase precursor | virulence |
| Lmo0204 | 639aa/actA: actin-assembly inducing protein precursor | virulence |
| Lmo0205 | 289aa/plcB: phospholipase C | virulence |
| Lmo0206 | 107aa/Unknwon | unknown |
| Lmo0252 | 128aa/Unknown, similar to repressor (penicilinase | Regulation |
| | repressor) | |
| Lmo0253 | 346aa/Unknown, similar to penicillinase antirepressor | Regulation |
| Lmo0255 | 167aa/Unknown, similar to unknown protein | unknown |
| Lmo0257 | 392aa/Unknown, similar to unknown protein | unknown |
| Lmo0262 | 490aa/inlG | cell surface proteins |
| Lmo0263 | 548aa/inlH: internalin H | cell surface proteins |
| Lmo0264 | 499aa/inlE: internalin E | cell surface proteins |
| Lmo0304 | 421aa/Unknwon | unknown |
| Lmo0310 | 358aa/unknown | unknown |
| Lmo0311 | 206aa/Unknwon | unknown |
| Lmo0312 | 400aa/Unknown, similar to unknown proteins | unknown |
| Lmo0313 | 248aa/Unknown, conserved hypothetical protein | unknown |
| Lmo0320 | 399aa/Unknown, similar to surface protein (peptidoglycan | cell surface proteins |
| | bound, LPXTG motif) | |
| Lmo0333 | 1778aa/Unknown, similar to internalin proteins, putative | cell surface proteins |
| | peptidoglycan bound protein (LPXTG motif) | |
| Lmo0334 | 174aa/Unknwon | unknown |
| Lmo0338 | 126aa/Unknwon | unknown |
| Lmo0378 | 148aa/Unknown | unknown |
| Lmo0379 | 151aa/Unknwon | unknown |
| Lmo0380 | 209aa/Unknwon | unknown |
| Lmo0381 | 128aa/Unknwon | unknown |
| Lmo0409 | 821aa/inIF internalin, similar to surface protein | cell surface proteins |
| | (peptidoglycan bound, LPXTG motif) | |
| Lmo0419 | 184aa/Unknown, similar to unknown protein | unknown |
| Lmo0433 | 800aa/inIA: Internalin A | cell surface proteins |
| Lmo0434 | 630aa/inIB: Internalin B | cell surface proteins |
| Lmo0440 | 167aa/Unknown | unknown |
| Lmo0444 | 926aa/Unknown, conserved hypothetical protein | unknown |
| Lmo0445 | 495aa/Unknown, similar to transcription regulator | Regulation |
| Lmo0446 | 329aa/Unknown, similar to penicillin acylase and to | Detoxification |
| | conjugated bile acid hydrolase | |
| Lmo0447 | 462aa/Unknown, similar to glutamate decarboxylase | Metabolism of amino acids and related molecules |
| Lmo0448 | 483/unknown, similar to amino acid antiporter | Transport/binding proteins and lipoproteins |
| Lmo0459 | 512aa/Unknown, similar to transcription regulator (VirR | Regulation |
| | from Streptococcus pyogenes) | |
| Lmo0460 | 689aa/Unknown, putative membrane associated lipoprotein | cell surface proteins |
| Lmo0461 | 157aa/Unknwon | unknown |
| Lmo0462 | 161aa/Unknwon | unknown |
| Lmo0463 | 101aa/putative peptidoglycan linked protein (LPXAG motif) | cell surface proteins |
| Lmo0466 | 567aa/Unknown | unknown |
| Lmo0467 | 107aa/Unknwon | unknown |
| Lmo0469 | 290aa/Unknwon | unknown |
| Lmo0470 | 209aa/Unknown, weakly similar to site-specific DNA- | DNA restriction/modification and repair |
| | methyltransferase | |
| Lmo0471 | 336aa/Unknwon | unknown |
| Lmo0472 | 295aa/Unknwon | unknown |
| Lmo0473 | 361aa/Unknwon | unknown |
| Lmo0474 | 115aa/Unknwon | unknown |
| Lmo0476 | 325aa/Unknown, similar to oxetanocin A resistance protein | Detoxification |
| | oxrB | |
| Lmo0477 | 125aa/Unknown, putative secreted protein | unknown |
| Lmo0478 | 125aa/Unknown, putative secreted protein | unknown |
| Lmo0492 | 302aa/Unknown, similar to transcriptional regulator (LysR | Regulation |
| | family) | |
| Lmo0493 | 586aa/Unknown, similar to acylase | Detoxification |
| Lmo0497 | 502aa/Unknown, similar to sugar transferase | Cell wall |
| Lmo0514 | 605aa/Unknown, similar to internalin proteins, similar to | cell surface proteins |
| | surface protein (peptidoglycan bound, LPXTG motif) | |
| Lmo0525 | 443aa/Unknwon | unknown |
| Lmo0549 | 673aa/Unknown, similar to internalin protein | cell surface proteins |
| Lmo0550 | 98aa/Unknown, pepdidoglycan bound protein (LPXTG | cell surface proteins |
| | motif) | |
| Lmo0610 | 589aa/Unknown, similar to internalin proteins, putative | cell surface proteins |
| | peptidoglycan bound protein (LPXTG motif) | |
| Lmo0630 | 676aa/Unknown, similar to transcription antiterminator | Regulation |
| | BgIG family | |
| Lmo0631 | 150aa/Unknown, similar to PTS system, fructose-specific | Transport/binding proteins and lipoproteins |
| | IIA component | |
| Lmo0632 | 353aa/Unknown, similar to PTS system, fructose-specific | Transport/binding proteins and lipoproteins |
| | IIC component | |
| Lmo0633 | 102aa/Unknown, similar to PTS system, fructose-specific | Transport/binding proteins and lipoproteins |
| | IIB component | |
| Lmo0634 | 422aa/unknown, similar to an E. coli putative tagatose 6- | Specific pathways |
| | phosphate kinase | |
| Lmo0638 | 209aa/Unknown | unknown |
| Lmo0673 | 69aa/Unknwon | unknown |
| Lmo0732 | 638aa/Unknown, putative peptidoglycan bound protein | cell surface proteins |
| | (LPXTG motif) | |
| Lmo0733 | 169aa/Unknown, similar to transcription regulator | Regulation |
| Lmo0734 | 334aa/Unknown, similar to transcriptional regulator (Lacl | Regulation |
| | family) | |
| Lmo0735 | 223aa/Unknown, similar to Ribulose-5-Phosphate 3- | Specific pathways |
| | Epimerase | |
| Lmo0736 | 148aa/Unknown, similar to ribose 5-phosphate isomerase | Specific pathways |
| Lmo0737 | 310aa/Unknwon | unknown |
| Lmo0738 | 617aa/Unknown, similar to phosphotransferase system | Transport/binding proteins and lipoproteins |
| | (PTS) beta-glucoside-specific enzyme IIABC component | |
| Lmo0739 | 457aa/Unknown, similar to 6-phospho-beta-glucosidase | Specific pathways |
| Lmo0745 | 167aa/Unknwon | unknown |
| Lmo0750 | 171aa/Unknwon | unknown |
| Lmo0751 | 98aa/Unknwon | unknown |
| Lmo0752 | 278aa/Unknown, weakly similar to a putative haloacetate | Detoxification |
| | dehalogenase | |
| Lmo0753 | 226aa/unknown, similar to transcription regulator Crp/Fnr | Regulation |
| | family | |
| Lmo0754 | 203aa/Unknown, weakly similar to a bile acid 7-alpha | Detoxification |
| | dehydratase | |
| Lmo0780 | 115aa/Unknown | unknown |
| Lmo0801 | 646aa/Unknown, similar to internalin, putative | cell surface proteins |
| | peptidoglycan bound protein (LPXTG motif) | |
| Lmo0804 | 156aa/Unknwon | unknown |
| Lmo0832 | 94aa/unknown, similar to transposase | Transposon and IS |
| Lmo0833 | 296aa/Unknown; similar to transcriptional regulator | Regulation |
| Lmo0834 | 237aa/Unknwon | unknown |
| Lmo0835 | 334aa/Unknown, putative peptidoglycan bound protein | cell surface proteins |
| | (LPXTG motif) | |
| Lmo0838 | 461aa/unknown, highly similar to hexose phosphate | Transport/binding proteins and lipoproteins |
| | transport protein | |
| Lmo0842 | 2044aa/Unknown, surface protein (LPXTG motif) | cell surface proteins |
| Lmo0843 | 85aa/unknown, similar to B. subtilis protein YsdA | unknown |
| Lmo0849 | 616aa/Unknown, similar to amidases | Cell wall |
| Lmo0880 | 462aa/Unknown, similar to wall associated protein | cell surface proteins |
| | precursor (LPXTG motif) | |
| Lmo0932 | 201aa/unknown, conserved hypothetical protein | unknown |
| Lmo0933 | 315aa/unknown, similar to sugar transferase | Cell wall |
| Lmo1030 | 342aa/unknown, similar to transcriptional regulator, Lacl | Regulation |
| | family | |
| Lmo1031 | 467aa/unknown, similar to hypothetical proteins | unknown |
| Lmo1032 | 274aa/unknown, similar to transketolase | Specific pathways |
| Lmo1033 | 318aa/unknown, similar to transketolase | Specific pathways |
| Lmo1034 | 487aa/unknown, similar to glycerol kinase | Specific pathways |
| Lmo1035 | 618aa/unknown, similar to phosphotransferase system | Transport/binding proteins and lipoproteins |
| | (PTS) beta-glucoside-specific enzyme IIABC | |
| Lmo1036 | 421aa/Unknwon | unknown |
| Lmo1060 | 220aa/unknown, similar to transcription response regulator | Regulation |
| Lmo1061 | 481aa/unknown, similar to sensor protein histidine kinases | Sensors (signal transduction) |
| | (2 components regulatory systems) | |
| Lmo1062 | 473aa/unknown, unknown, similar to ABC transporters | Transport/binding proteins and lipoproteins |
| | (permease protein) | |
| Lmo1063 | 222aa/unknown, similar to ABC transporter (ATP binding | Transport/binding proteins and lipoproteins |
| | protein) | |
| Lmo1076 | 572aa/unknown, similar to AUTOLYSIN (EC 3.5.28) (N- | Cell wall |
| | ACETYLMURAMOYL-L-ALANINE AMIDASE) | |
| Lmo1077 | 562aa/unknown, similar to teichoic acid biosynthesis | Cell wall |
| | protein B | |
| Lmo1079 | 952aa/unknown, similar to B. subtilis YfhO protein | unknown |
| Lmo1080 | 623aa/unknown, similar to B. subtilis minor teichoic acids | Cell wall |
| | biosynthesis protein GgaB | |
| Lmo1081 | 288aa/Unknown, similar to glucose-1-phosphate thymidyl | Cell wall |
| | transferase | |
| Lmo1082 | 186aa/Unknown, similar to dTDP-sugar epimerase | Cell wall |
| Lmo1083 | 328aa/Unknown, similar to dTDP-D-glucose 4,6-dehydratase | Cell wall |
| Lmo1084 | 276aa/unknown, similar to DTDP-L-rhamnose synthetase | Cell wall |
| Lmo1085 | 696aa/unknown, similar to teichoic acid biosynthesis | Cell wall |
| | protein B | |
| Lmo1088 | 386aa/tagB: unknown, similar to teichoic acid | Cell wall |
| | biosynthesis protein B precursor | |
| Lmo1090 | 327aa/unknown, similar to glycosyltransferases | Cell wall |
| Lmo1091 | 337aa/unknown, similar to glysosyltransferases | Cell wall |
| Lmo1097 | 400aa/unknown, similar to integrases | Transposon and IS |
| Lmo1099 | 139aa/unknown, similar to a protein encoded by Tn916 | Transposon and IS |
| Lmo1101 | 166aa/Unknown, similar to lipoprotein signal peptidase | Protein secretion |
| Lmo1102 | 111aa/unknown, similar to cadmium efflux system | Detoxification |
| | accessory proteins | |
| Lmo1103 | 305aa/unknown, highly similar to TN916 ORF13 | Transposon and IS |
| Lmo1104 | 336aa/unknown, highly similar to TN916 ORF14 and to | Cell wall |
| | L. monocytogenes P60 protein | |
| Lmo1105 | 719aa/unknown, highly similar to TN916 ORF15 | Transposon and IS |
| Lmo1106 | 816aa/unknown, highly similar to TN916 ORF16 | Transposon and IS |
| Lmo1107 | 131aa/unknown, highly similar to TN916 ORF17 | Transposon and IS |
| Lmo1108 | 166aa/unknown, highly similar to TN916 ORF18 | Transposon and IS |
| Lmo1110 | 96aa/unknown, similar to unknown proteins | unknown |
| Lmo1111 | 394aa/unknown, highly similar to TN916 ORF20 | Transposon and IS |
| Lmo1112 | 466aa/unknown, highly similar to TN916 ORF21 | Transposon and IS |
| Lmo1113 | 124aa/unknown, highly similar to TN916 ORF22 | Transposon and IS |
| Lmo1114 | 104aa/unknown, highly similar to TN916 ORF23 | Transposon and IS |
| Lmo1115 | 862aa/unknown, similar to fibrinogen-binding protein | cell surface proteins |
| | (LPXTG motif) | |
| Lmo1116 | 257aa/unknown, similar to regulatory proteins | Regulation |
| Lmo1117 | 118aa/Uknwon | unknown |
| Lmo1118 | 505aa/Unknwon | unknown |
| Lmo1119 | 285aa/unknown, similar to methylases | DNA restriction/modification and repair |
| Lmo1120 | 138aa/Unknwon | unknown |
| Lmo1121 | 122aa/Unknwon | unknown |
| Lmo1125 | 177aa/unknown | unknown |
| Lmo1133 | 102aa/unknown, similar to B. subtilis YjcS protein | unknown |
| Lmo1134 | 115aa/unknown, similar to regulatory proteins | Regulation |
| Lmo1135 | 383aa/Unknwon | unknown |
| Lmo1136 | 539aa/Unknown, similar to internalin, putative | cell surface proteins |
| | peptidoglycan bound protein (LPXTG motif) | |
| Lmo1247 | 87aa/Unknwon | unknown |
| Lmo1263 | 71aa/unknown, similar to transcriptional regulator | Regulation |
| Lmo1289 | 593aa/Unknown, similar to internalin proteins, putative | cell surface proteins |
| | peptidoglycan bound protein (LPXTG motif) | |
| Lmo1302 | 204aa/unknown, highly similar to SOS response regulator | Regulation |
| | lexA, transcription repressor protein | |
| Lmo1303 | 109aa/unknown, similar to B. subtilis YneA protein | unknown |
| Lmo1307 | 280aa/Unknwon | unknown |
| Lmo1413 | 439aa/Unknown, putative peptidoglycan bound protein | cell surface proteins |
| | (LPXTG motif) | |
| Lmo1441 | 368aa/Unknown, similar to putative peptidoglycan | Cell wall |
| | acetylation protein | |
| Lmo1451 | 331aa/Unknown, similar to E. coli LytB protein | unknown |
| Lmo1478 | 145aa/Unknown, similar to transcriptional regulator (MerR | Regulation |
| | family) | |
| Lmo1597 | 259aa/Unknwon | unknown |
| Lmo1604 | 181aa/unknown | unknown |
| Lmo1648 | 120aa/Unknwon | unknown |
| Lmo1656 | 143aa/Unknwon | unknown |
| Lmo1666 | 1711aa/unknown, peptidoglycan linked protein (LPxTG) | cell surface proteins |
| Lmo1700 | 68aa/Unknwon | unknown |
| Lmo1707 | 73aa/Unknwon | unknown |
| Lmo1714 | 190aa/Unknwon | unknown |
| Lmo1786 | 296aa/inIC | cell surface proteins |
| Lmo1799 | 903aa/Unknown, putative peptidoglycan bound protein | cell surface proteins |
| | (LPXTG motif) | |
| Lmo1968 | 247aa/unknown, similar to creatinine amidohydrolases | Metabolism of amino acids and related molecules |
| Lmo1969 | 202aa/Unknown, similar to 2-keto-3-deoxygluconate-6- | Specific pathways |
| | phosphate aidolase | |
| Lmo1970 | 305aa/Unknown, similar to putative phosphotriesterase | Metabolism of lipids |
| | related proteins | |
| Lmo1971 | 422aa, Unknown, similar to pentitol PTS system enzyme II C | Transport/binding proteins and lipoproteins |
| | component | |
| Lmo1973 | 147aa/Unknown, similar to PTS system enzyme II A | Transport/binding proteins and lipoproteins |
| | component | |
| Lmo1974 | 249aa/unknown, similar to transcription regulators, (GntR | Regulation |
| | family) | |
| Lmo2026 | 626aa/unknown, peptidoglycan linked protein (LPxTG) | cell surface proteins |
| Lmo2027 | 367aa/Unknown, putative cell surface protein, similar to | cell surface proteins |
| | internalin proteins | |
| Lmo2045 | 128aa/Unknwon | unknown |
| Lmo2067 | 325aa/Unknown, similar to conjugated bile acid hydrolase | Detoxification |
| Lmo2085 | 562aa/Unknown, putative peptidoglycan bound protein | cell surface proteins |
| | (LPXTG motif) | |
| Lmo2143 | 580aa/Unknown, weakly similar to mannose-6-phosphate | Specific pathways |
| | isomerase | |
| Lmo2144 | 244aa/Unknown, similar to transcription regulator GntR | Regulation |
| | family | |
| Lmo2157 | 633aa/SepA-Unknown | unknown |
| Lmo2178 | 1612aa/Unknown, putative peptidoglycan bound protein | cell surface proteins |
| | (LPXTG motif) | |
| Lmo2197 | 114aa/Unknwon | unknown |
| Lmo2276 | 165aa/Unknown, similar to an unknown bacteriophage | Phage-related functions |
| | protein | |
| Lmo2277 | 183aa/Unknwon | unknown |
| Lmo2278 | 281aa/LysA: L-alanoyl-D-glutamate peptidase | Phage-related functions |
| Lmo2282 | 105aa/protein gp21 [Bacteriophage A118] | Phage-related functions |
| Lmo2283 | 357aa/protein gp20 [Bacteriophage A118] | Phage-related functions |
| Lmo2296 | 333aa/Unknown, similar to coat protein [Bacteriophage | Phage-related functions |
| | SPP1] | |
| Lmo2301 | 264aa/Unknown, similar to putative terminase small subunit | Phage-related functions |
| | from Bacteriophage A118 | |
| Lmo2302 | 179aa/unknown | unknown |
| Lmo2311 | 153aa/Unknwon | unknown |
| Lmo2312 | 156aa/Unknwon | unknown |
| Lmo2313 | 147aa/Unknown, similar to a bacteriophage protein | Phage-related functions |
| Lmo2318 | 232aa/Unknwon | unknown |
| Lmo2319 | 158aa/Unknown, similar to bacteriophage proteins | Phage-related functions |
| Lmo2322 | 71aa/gp44 [Bacteriophage A118] | Phage-related functions |
| Lmo2324 | 258aa/Unknown, similar to anti-repressor [Bacteriophage | Regulation |
| | A118] | |
| Lmo2331 | 226aa/Unknown, weakly similar to gp32_Bacteriophage | Phage-related functions |
| | A118 protein | |
| Lmo2387 | 408aa/Unknown, conserved hypothetical protein | unknown |
| Lmo2408 | 67aa/Unknown, similar to repressor protein | Regulation |
| Lmo2409 | 137aa/Unknwon | unknown |
| Lmo2410 | 183aa/unknown | unknown |
| Lmo2443 | 139aa/Unknwon | unknown |
| Lmo2451 | 77aa/Unknown, similar to preprotein translocase subunit | Protein secretion |
| | SecG | |
| Lmo2548 | 81aa/rpmE: ribosomal protein L31 | Ribosomal proteins |
| Lmo2567 | 66aa/Unknwon | unknown |
| Lmo2576 | 1530aa/Unknwon, peptidoglycan anchored protein (LPXTG | cell surface proteins |
| | motif) | |
| Lmo2594 | 119aa/Unknwon | unknown |
| Lmo2595 | 289aa/Unknown, similar to unknown proteins | unknown |
| Lmo2671 | 122aa/Unknwon | unknown |
| Lmo2686 | 177aa/Unknwon | unknown |
| Lmo2731 | 122aa/unknown, similar to transcription regulator (RpiR | Regulation |
| | family) | |
| Lmo2732 | 250aa/Unknwon | unknown |
| Lmo2733 | 652aa/Unknown, similar to PTS system, fructose-specific | Transport/binding proteins and lipoproteins |
| | IIABC component | |
| Lmo2734 | 882aa/Unknown, weakly similar to sugar hydrolase | Specific pathways |
| Lmo2735 | 560aa/Unknown, similar to Sucrose phosphorylase | Specific pathways |
| Lmo2736 | 377aa/Unknown, conserved hypothetical protein | unknown |
| Lmo2771 | 482aa/Unknown, similar to beta-glucosidase | Specific pathways |
| Lmo2772 | 617aa/Unknown, similar to beta-glucoside-specific enzyme | Transport/binding proteins and lipoproteins |
| | IIABC | |
| Lmo2773 | 279aa/Unknwon, similar to transcription antiterminator | Regulation |
| Lmo2776 | 131aa/Unknwon | unknown |
| Lmo2781 | 756aa/Unknown, similar to beta-glucosidase | Specific pathways |
| Lmo2782 | 99aa/Unknown, similar to PTS, cellobiose-specific IIB | Transport/binding proteins and lipoproteins |
| | component | |
| Lmo2786 | 327aa/bvrC: unknown | unknown |
| Lmo2787 | 640aa/bvrB-beta-glucoside-specific phosphotransferase | Transport/binding proteins and lipoproteins |
| | enzyme II | |
| Lmo2807 | 127aa/Unknown, hypothetical secreted protein | unknown |
| Lmo2809 | 126aa/Unknown, hypothetical secreted protein | unknown |
| Lmo2821 | 851aa/Unknown, similar to internalin, Unknown, putative | Cell surface proteins |
| | peptidoglycan bound protein (LPXTG motif) | |

L. innocua (CLIP11262) genes

| | | |
|---|---|---|
| Lin0035 | 219aa/Unknown, similar to E. coli DedA protein | unknown |
| Lin0038 | 179aa/unknown, highly similar to single-strand binding | DNA replication |
| | protein (SSB) | |
| Lin0060 | 204aa/unknown | unknown |
| Lin0062 | 210aa/unknown, hypothetical protein | unknown |
| Lin0064 | 129aa/Unknwon | unknown |
| Lin0065 | 136aa/Unknwon | unknown |
| Lin0072 | 216aa/Unknwon | unknown |
| Lin0073 | 158aa/Unknown, similar to a putative repressor protein | Regulation |
| | [Bacteriophage A118] | |
| Lin0197 | 358aa/unknown, similar to chloromuconate cycloisomerase | Metabolism of carbohydrates (Specific |
| | ykfB of B. subtilis | pathways) |
| Lin0198 | 292aa/unknown, P45 related protein | Cell wall |
| Lin0199 | 257aa/unknown, some similarities to probable beta-lactamase | Detoxification |
| Lin0307 | 219aa/unknown, similar to ABC transporters (ATP-binding | Transport/binding proteins and lipoproteins |
| | protein) | |
| Lin0308 | 261aa/unknown, similar to hypothetical proteins | unknown |
| Lin0332 | 405aa/unknown, similar to putative permeases | Transport/binding proteins and lipoproteins |
| Lin0338 | 266aa/Unknwon | unknown |
| Lin0372 | 656aa/unknown, probable cell surface protein (LPXTG motif) | cell surface proteins |
| Lin0397 | 120aa/Unknwon | unknown |
| Lin0398 | 536aa/Unknwon | unknown |
| Lin0399 | 252aa/Unknwon | unknown |
| Lin0415 | 410aa/unknown, probable cell surface protein (LPXTG motif) | cell surface proteins |
| Lin0453 | 169aa/unknown | unknown |
| Lin0454 | 2167aa/unknown, similar to cell wall-associated protein | cell surface proteins |
| | precursor wapA (B. subtilis) | |
| Lin0455 | 184aa/Unknwon | unknown |
| Lin0465 | 212aa/unknown, conserved hypothetical protein, similar to | unknown |
| | yoaZ B. subtilis | |
| Lin0476 | 350aa/Unknwon | unknown |
| Lin0478 | 407aa/Unknwon | unknown |
| Lin0559 | 96aa/unknown, probable cell surface protein (LPXTG motif) | cell surface proteins |
| Lin0661 | 596aa/unknown, internalin like protein (LPXTG motif) | cell surface proteins |
| Lin0665 | 831aa/unknown, highly similar to ORFA of Listeria seeligeri, | cell surface proteins |
| | (LPXTG motif) | |
| Lin0677 | 293aa/unknown, conserved hypothetical protein | unknown |
| Lin0739 | 521aa/unknown, internalin like protein (LPXTG motif) | cell surface proteins |
| Lin0740 | 657aa/unknown, probable cell surface protein (LPXTG motif) | Cell surface proteins |
| Lin0772 | 417aa/Unknwon | unknown |
| Lin0801 | 240aa/unknown, similar to two-component response | RNA synthesis (Regulation) |
| | regulators | |
| Lin0802 | 433aa/unknown, similar to two-component sensor histidine | Sensors (signal transduction) |
| | kinases | |
| Lin0803 | 1386Unknown, surface protein (LPXTG motif) | cell surface proteins |
| Lin0833 | 489aa/Unknwon | unknown |
| Lin0834 | 369aa/unknown, some similarities to hypothetical proteins | unknown |
| Lin0835 | 285aa/Unknwon | unknown |
| Lin0865 | 131aa/unknown, hypothetical protein | unknown |
| Lin0867 | 421aa/Unknwon | unknown |
| Lin0868 | 378aa/Unknwon | unknown |
| Lin1065 | 668aa/unknown, hypothetical protein | unknown |
| Lin1066 | 329aa/unknown, similar to dolichol phosphate mannose synthase | Cell wall |
| Lin1067 | 696aa/unknown | unknown |
| Lin1067 | 774aa/unknown, similar to hypothetical protein 3 (capsulation | Cell wall |
| | locus) of Haemophilus influenzae | |
| Lin1068 | | |
| Lin1069 | 336aa/Unknwon | unknown |
| Lin1073 | 462aa/unknown, similar to galactosamine-containing minor | Cell wall |
| | teichoic acid biosynthesis protein GgaA | |
| Lin1082 | 387aa/Unknwon | unknown |
| Lin1083 | 216aa/Unknwon | unknown |
| Lin1084 | 94aa/Unknwon | unknown |
| Lin1090 | 111aa/Unknwon | unknown |
| Lin1099 | 235aa/Unknwon | unknown |
| Lin1204 | 1237aa/unknown, similar to internalin proteins (LPXTG motif) | Cell surface proteins |
| Lin1211 | 486aa/unknown | unknown |
| Lin1212 | 184aa/Unknwon | unknown |
| Lin1241 | | |
| Lin1258-57 | 184aa-175aa/Unknown | unknown |
| Lin1260 | 255aa/unknown, similar to probable antirepressor - Bacillus | Phage-related functions |
| | subtilis phage SPBc2 | |
| Lin1262 | 445aa/Unknwon | unknown |
| Lin1299 | 121aa/Unknwon | unknown |
| Lin1300 | 203aa/Unknwon | unknown |
| Lin1328 | 620aa/unknown, internalin like protein (LPXTG motif) | Cell surface proteins |
| Lin1452 | 125aa/Unknwon | unknown |
| Lin1621 | 253aa/unknown, similar to ketoacyl reductases | Metabolism of lipids |
| Lin1723 | 199aa/Unknwon | unknown |
| Lin1728 | 369aa/unknown, similar to hypothetical proteins | unknown |
| Lin1731 | 461aa/unknown, some similarities to conserved hypothetical | unknown |
| | proteins | |
| Lin1744 | 453aa/Unknwon | unknown |
| Lin1745 | 140aa/Unknown | unknown |
| Lin1747 | 125aa/Unknwon | unknown |
| Lin1756 | 660aa/unknown, similar to hypothetical protein of | unknown |
| | Staphylococcus aureus phage phi PVL | |
| Lin1811 | 323aa/unknown, similar to unknown proteins | unknown |
| Lin1813 | 620aa/Unknown, similar to excinuclease ABC subunit A | DNA restriction/modification and repair |
| Lin1898 | 177aa/Unknown, similar to putative NAD(P)H oxidoreductase | Membrane bioenergetics (electron transport |
| | | chain and ATP synthase) |
| Lin2100 | 764aa/unknown, similar to p60-related proteins | Cell wall |
| Lin2373 | 298aa/Unknown, similar to AbiD phage protein | Phage-related functions |
| Lin2383 | 1601unknown, similar to hypothetical protein [Lactobacillus | Phage-related functions |
| | casei bacteriophage A2] | |
| Lin2394 | 439aa/Unknown | unknown |
| Lin2397 | 190aa/unknown, similar to sigma factor-like positive control | RNA synthesis (Initiation) |
| | protein of B. subtilis | |
| Lin2405 | 69aa/Unknwon | unknown |
| Lin2425 | 235aa/Unknwon | unknown |
| Lin2494 | 124aa/unknown, hypothetical protein | unknown |
| Lin2603 | 95aa/Unknwon | unknown |
| Lin2604 | 80aa/Unknwon | unknown |
| Lin2693 | 497aa/Unknwon | unknown |
| Lin2724 | 938aa/unknown, internalin-like protein (LPXTG motif) | cell surface proteins |
| Lin2741 | 152aa/Unknwon | unknown |
| Lin2743 | 98aa/Unknwon | unknown |
| Lin2744 | 1334aa/unknown, similar to hypothetical proteins | unknown |
| Lin2839 | 99aa/Unknwon | unknown |
| Lin2918 | 159aa/Unknwon | unknown |
| Lin2941 | 663aa/Unknwon | unknown |
| Lin2945 | 191aa/Unknwon | unknown |

L. monocytogenes (CLIP 80459) genes

| | | |
|---|---|---|
| ORF 2541 | 590bp/unknwon, similar to internalin proteins, putative | cell surface proteins |
| | peptidoglycan bound protein (LPXTG motif) | |
| ORF 2017 | 285bp/unknwon, similar to internalin proteins, putative | cell surface proteins |
| | peptidoglycan bound protein (LPXTG motif) | |
| ORF 3401 | 575bp/unknown | unknown |
| ORF 2074 | 599bp/unknown similar to oligo-1,6-glucosidase | Specific pathways |
| ORF 528-529 | 591bp/unknown-unknown | unknown |
| ORF 2815 | 603bp/unknown, similar to heat shock protein HtpG | unknown |
| ORF 2568-2569 | 597bp/unknwon, similar to internalin proteins, putative | cell surface proteins |
| | peptidoglycan bound protein (LPXTG motif)-unknown | |
| ORF 2847 | 590bp/similar to phage integrase | Phage-related functions |
| ORF 1388-1387 | 600bp/unknown-unknown | unknown |
| ORF 799 | 453bp/unknown | unknown |
| ORF 72-73-74 | 598bp/unknown-unknown-unknown | unknown |
| ORF 1912-1913 | 602bp/unknown-unknown | unknown |
| ORF 150 | 393bp/unknown, similar to amidase | cell wall |
| ORF 2777 | 600bp/unknown | unknown |
| ORF 2579 | 601bp/unknown, sucrose phosphorylase | Specific pathways |
| ORF 75 | 504bp/unknown | unknown |
| ORF 660 | 603bp/unknown | unknown |
| ORF 2884-2885 | 592bp/unknown | unknown |
| ORF 2885 | 592bp/unknown | unknown |
| ORF 1445 | 607bp/unknown, conserved hypothetical protein ykcB - | unknown |
| | Bacillus subtilis | |
| ORF 1590 | 588bp/unknwon, similar to internalin proteins, putative | cell surface proteins |
| | peptidoglycan bound protein (LPXTG motif) | |
| ORF 182 | 468bp/unknown, similar to B. subtilis YukA, | unknown |
| ORF 1761 | 599bp/unknwon, similar to internalin proteins, putative | cell surface proteins |
| | peptidoglycan bound protein (LPXTG motif) | |
| ORF 105 | 599bp/unknown, similar to similar to ABC transporter | Transport/binding proteins |
| | | and lipoproteins |
| ORF 2372 | 596bp/unknown, similar to teichoic acid biosynthesis | cell wall |
| | protein C | |
| ORF 2110 | 597bp/unknown, similar to hypothetical protein | unknown |
| ORF 1491 | 598bp/unknown, similar to hypothetical protein | unknown |
| ORF 1490 | 600bp/unknown, similar to sucrose-specific PTS system | Transport/binding proteins |
| | IIBC component | and lipoproteins |
| ORF 1390 | 624bp/unknown, similar to hypothetical protein | unknown |
| ORF 1391 | 600bp/unknown | unknown |
| ORF 1228 | 599bp/unknown, similar to similar to ABC transporter | Transport/binding proteins |
| | | and lipoproteins |
| ORF 29 | 609bp/unknwon, similar to internalin proteins, putative | cell surface proteins |
| | peptidoglycan bound protein (LPXTG motif) | |
| ORF 3792 | 594bp/unknown | unknown |
| ORF 3840 | 593bp/unknown, similar to transcription regulator | Regulation |
| ORF 4101 | 613bp/unknown, similar to similar to ABC transporter | Transport/binding proteins |
| | | and lipoproteins |
| ORF 3103 | 600bp/unknown, similar to peptidoglycan lytic protein P45 | cell wall |
| ORF 1490 | 474bp/unknown, similar to sucrose phosphotransferase | Transport/binding proteins |
| | enzyme IIBC component | and lipoproteins |
| ORF 2217 | 602bp/unknown, imilar to type II DNA modification enzyme | DNA |
| | | restriction/modification |
| | | and repair |
| ORF 2939-2968 | 583bp/unknown-unknown | unknown |
| ORF 4457 | 588bp/unknown, DNA double-strand break repair rad50 | DNA |
| | ATPase | restriction/modification |
| | | and repair |
| ORF 3184 | 597bp/unknown | unknown |
| ORF 148-149 | 620bp/unknown-unknown, similar to amidase | cell wall |
| ORF 3157 | 589bp/unknown, similar to phage protein | Phage-related functions |
| ORF 2858 | 249bp/unknown, similar to hypothetical protein | unknown |
| ORF 1384-1385 | 422bp/unknown-unknown | unknown |
| ORF 2541 | 351bp/unknwon, similar to internalin proteins, putative | cell surface proteins |
| | peptidoglycan bound protein (LPXTG motif) | |
| ORF 2819 | 562bp/unknown, similar to transcriptional regulator | Regulation |
| ORF 2262 | 601bp/unknown | unknown |
| ORF 817 | 594bp/unknown | unknown |
| ORF 757 | 522bp/unknown | unknown |
| ORF 2013 | 595bp/unknwon, similar to internalin proteins, putative | cell surface proteins |
| | peptidoglycan bound protein (LPXTG motif) | |
| ORF 2076 | 597bp/unknown, similar to maltodextrin ABC transporter | Transport/binding proteins |
| | | and lipoproteins |
| ORF 2013 | 597bp/unknwon, similar to internalin proteins, putative | cell surface proteins |
| | peptidoglycan bound protein (LPXTG motif) | |
| dnaN (a) | 381aa/DNA polymerase III, beta chain | DNA replication |
| dnaN (b) | | |
| dnaN (c) | 381aa/DNA polymerase III, beta chain | DNA replication |
| dnaN (d) | | |
| gyrB (a) | 646aa/DNA gyrase subunit B | DNA packaging and |
| | | segregation |
| gyrB (b) | | |
| gyrB (c) | 646aa/DNA gyrase subunit B | DNA packaging and |
| | | segregation |
| gyrB (d) | | |
| rpoB (a) | 1184aa/RNA polymerase (beta subunit) | Elongation |
| rpoB (b) | | |
| rpoB (c) | 1184aa/RNA polymerase (beta subunit) | Elongation |
| rpoB (d) | | |
| rpoC (a) | 1201aa/RNA polymerase (beta subunit) | Elongation |
| rpoC (b) | | |
| rpoC (c) | 1201aa/RNA polymerase (beta subunit) | Elongation |
| rpoC (d) | | |

TABLE 9
L. monocytogenes specific marker genes

| | | L. monocytogenes | | | | |
|---|---|---|---|---|---|---|
| | | 1/2a, 1/2b, 1/2c, | | | L. ivanovii, L. innocua, | |
| | | 3a, 3b, 3c, 7, 4b, | | | L. welshimeri, L seeligeri | |
| | | 4d, 4e | 4c | 4a | L. grayi | |
| | Gene name | (88 strains) | (2 strains) | (3 strains) | (20 strains) | Functional category |
| I | Lmo 0082 | 88 | 2 | 3 | 0 | Unknown |
| | Lmo 0083 | 88 | 2 | 3 | 0 | Regulation |
| | plcA | 88 | 2 | 3 | 0 | Virulence |
| | actA | 88 | 2 | 3 | 0 | Virulence |
| | plcB | 88 | 2 | 3 | 0 | Virulence |
| | Lmo206 | 88 | 2 | 3 | 0 | Unknown |
| | inlA | 88 | 2 | 3 | 0 | Cell surface proteins |
| | inlB | 88 | 2 | 3 | 0 | Cell surface proteins |
| | Lmo 0440 | 88 | 2 | 3 | 0 | Unknown |
| | Lmo 0638 | 88 | 2 | 3 | 0 | Unknown |
| | Lmo 0733 | 88 | 2 | 3 | 0 | Regulation |
| | Lmo 0833 | 88 | 2 | 3 | 0 | Regulation |
| | Lmo 1133 | 88 | 2 | 3 | 0 | Unknown |
| | Lmo 1135 | 88 | 2 | 3 | 0 | Unknown |
| | Lmo 2732 | 88 | 2 | 3 | 0 | Unknown |
| | Lmo 2733 | 88 | 2 | 3 | 0 | Transport/binding proteins and lipoproteins |
| | Lmo 2734 | 88 | 2 | 3 | 0 | Specific pathways |
| | Lmo 2736 | 88 | 2 | 3 | 0 | Unknown |
| II | Lmo0017 | 88 | 2 | 0 | 0 | Cell wall |
| | Lmo0094 | 88 | 0 | 0 | 0 | Unknown |
| | inlH | 88 | 2 | 0 | 0 | Cell surface proteins |
| | inlE | 88 | 2 | 0 | 0 | Cell surface proteins |
| | Lmo0333 | 88 | 2 | 0 | 0 | Cell surface proteins |
| | Lmo0549 | 88 | 2 | 0 | 0 | Soluble internalins |
| | Lmo0832 | 88 | 0 | 0 | 0 | Transposon and IS |
| | Lmo0834 | 88 | 2 | 0 | 0 | Unknown |
| | Lmo0835 | 88 | 0 | 0 | 0 | Cell surface proteins |
| | Lmo1441 | 88 | 0 | 0 | 0 | Cell wall |
| | Lmo1451 | 88 | 0 | 0 | 0 | Unknown |
| | Lmo2821 | 88 | 2 | 0 | 0 | Cell surface proteins |

TABLE 10
L. monocytogenes lineage specific marker genes

| | | Lineage I | | Lineage II | | Lineage III | | |
|---|---|---|---|---|---|---|---|---|
| | | | I. 2 | II. 1 | | | III. 2 | |
| | | I. 1 | (1/2c, 3c) | (4b, 4d, 4e) | II. 2 | III. 1 | (4c) | |
| | Gene | (1/2a, 3a) | (12 | (27 | (1/2b, 3b) | (4a) | (2 | |
| | name | (27strains) | strains) | strains) | (20strains) | (3 strains) | strains) | Functional category |
| A | Lmo 0171 | 27 | 12 | 0 | 0 | 0 | 0 | Cell surface proteins |
| | Lmo 0172 | 27 | 12 | 0 | 0 | 0 | 0 | Transposon and IS |
| | Lmo 0525 | 27 | 12 | 0 | 0 | 0 | 0 | Unknown |
| | Lmo 0734 | 27 | 12 | 0 | 0 | 0 | 0 | Regulation |
| | Lmo 0735 | 27 | 12 | 0 | 0 | 0 | 0 | Specific pathways |
| | Lmo 0736 | 27 | 12 | 0 | 0 | 0 | 0 | Specific pathways |
| | Lmo 0737 | 27 | 12 | 0 | 0 | 0 | 0 | Unknown |
| | Lmo 0738 | 27 | 12 | 0 | 0 | 0 | 0 | Transport/binding proteins and lipoproteins |
| | Lmo 0739 | 27 | 12 | 0 | 0 | 0 | 0 | Specific pathways |
| | Lmo 1060 | 27 | 12 | 0 | 0 | 0 | 0 | Regulation |
| | Lmo 1061 | 27 | 12 | 0 | 0 | 0 | 0 | Sensors |
| | Lmo 1062 | 27 | 12 | 0 | 0 | 0 | 0 | Transport/binding proteins and lipoproteins |
| | Lmo 1063 | 27 | 12 | 0 | 0 | 0 | 0 | Transport/binding proteins and lipoproteins |
| | Lmo 1968 | 27 | 12 | 0 | 0 | 0 | 0 | Metabolism of amino acids |
| | Lmo 1969 | 27 | 12 | 0 | 0 | 0 | 0 | Specific pathways |
| | Lmo 1970 | 27 | 12 | 0 | 0 | 0 | 0 | Metabolism of lipids |
| | Lmo 1971 | 27 | 12 | 0 | 0 | 0 | 0 | Transport/binding proteins and lipoproteins |
| | Lmo 1973 | 27 | 12 | 0 | 0 | 0 | 0 | Transport/binding proteins and lipoproteins |
| | Lmo 1974 | 27 | 12 | 0 | 0 | 0 | 0 | Regulation |
| | bvrC | 27 | 12 | 0 | 0 | 0 | 2 | Unknown |
| | bvrB | 27 | 12 | 0 | 0 | 0 | 2 | Transport/binding proteins and lipoproteins |
| B | Lmo 0151 | 3 | 12 | 0 | 0 | 0 | 0 | Unknown |
| | Lmo 0466 | 2 | 12 | 0 | 0 | 0 | 0 | Unknown |
| | Lmo 0467 | 2 | 12 | 0 | 0 | 0 | 0 | Unknown |
| | Lmo 0469 | 2 | 12 | 0 | 0 | 0 | 0 | Unknown |
| | Lmo 0470 | 2 | 12 | 0 | 0 | 0 | 0 | DNA restrictions and modifications |
| | Lmo 0471 | 2 | 12 | 0 | 0 | 0 | 0 | Unknown |
| | Lmo 1118 | 1 | 12 | 0 | 0 | 0 | 0 | Unknown |
| | Lmo 1119 | 1 | 12 | 0 | 0 | 0 | 0 | DNA restrictions and modifications |
| C | ORF2819 | 0 | 0 | 27 | 20 | 0 | 0 | Unknown, Similar to hypothetical |
| | | | | | | | | transcriptional regulator |
| | ORF3840 | 0 | 0 | 27 | 20 | 0 | 0 | Unknown, similar to transcriptional regulator |
| | ORF2568 | 0 | 0 | 27 | 20 | 0 | 0 | Unknown, Similar to internalin proteins, |
| | | | | | | | | putative peptidoglycan bound protein |
| | | | | | | | | (LPXTG) |
| | ORF1761 | 0 | 0 | 27 | 20 | 0 | 0 | Unknown, Similar to internalin proteins, |
| | | | | | | | | putative peptidoglycan bound protein |
| | | | | | | | | (LPXTG) |
| | ORF0029 | 0 | 0 | 27 | 19 | 0 | 0 | Unknown, Similar to internalin proteins, |
| | | | | | | | | putative peptidoglycan bound protein |
| | | | | | | | | (LPXTG) |
| D | ORF0799 | 0 | 0 | 27 | 0 | 0 | 0 | Unknown |
| | ORF2372 | 0 | 0 | 27 | 0 | 0 | 0 | Unknown, Similar to teichoic acid protein |
| | | | | | | | | precurser C |
| | ORF2110 | 0 | 0 | 27 | 0 | 0 | 0 | Unknown, Putative secreted protein |

TABLE 11
Distribution of cell surface proteins

| | L. monocytogenes | | | | | | | | | | |
|---|---|---|---|---|---|---|---|---|---|---|---|
| | Lineage I | | Lineage II | | Lineage III | | | | | | |
| | I.1 | I.2 | II.1 | II.2 | III.1 | III.2 | | | | | |
| | (1/2a,3a) | (1/2c,3c) | (4b,4d,4e) | (1/2b,3b, | (4c) | (4a) | L. | L. | L. | L. | L. |
| | (27 | (12 | (27 | 7)(20 | (2 | (3 | innocua | ivanovii | seeligeri | welshimeri | grayi |
| Gene | strains) | strains) | strains) | strains) | strains) | strains) | (8 strains) | (6 strains) | (2 strains) | (2 strains) | (2 strains) |
| inlA | 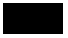 | 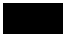 | 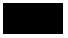 | 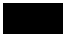 | 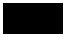 | 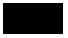 | − | − | − | − | − |
| inlB | 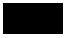 | 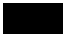 | 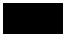 | 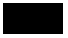 | 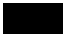 | 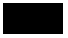 | − | − | − | − | − |
| Lmo2085 | 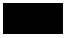 | 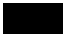 | 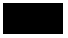 | 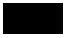 | 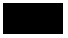 | 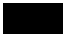 | − | − | +/− | +/− | − |
| Lmo1413 | 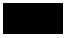 | 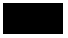 | 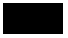 | 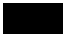 | 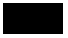 | 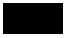 | − | − | +/− | − | − |
| Lmo0463 | 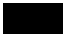 | 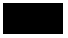 | 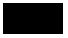 | 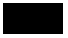 | 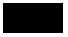 | 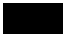 | − | − | − | − | − |
| Lmo0460 | 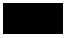 | 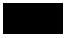 | 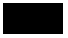 | 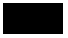 | 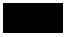 | 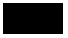 | − | − | − | − | − |
| Lmo0550 | 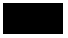 | 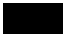 | 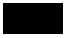 | 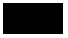 | 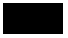 | 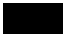 | −* | − | − | − | − |
| Lmo0160 | 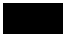 | 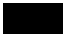 | 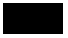 | 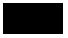 | 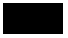 | 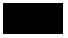 | −* | − | − | − | − |
| Lmo0333 | 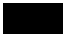 | 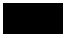 | 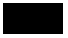 | 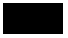 | 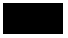 | − | − | − | − | − | − |
| inlE | 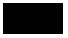 | 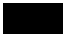 | 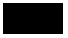 | 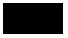 | 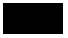 | − | − | − | − | − | − |
| inlH | 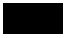 | 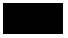 | 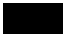 | 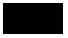 | 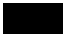 | − | − | − | − | − | − |
| Lmo2821 | 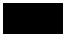 | 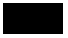 | 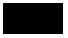 | 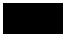 | 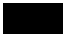 | − | − | − | − | − | − |
| Lmo2027 | 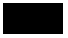 | 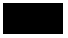 | 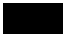 | 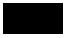 | 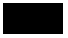 | − | − | − | − | − | − |
| inlC | 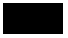 | 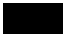 | 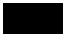 | 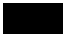 | 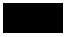 | − | − | + | − | − | − |
| Lmo0835 | 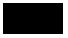 | 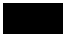 | 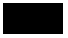 | 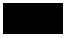 | − | − | − | − | − | − | − |
| Lmo1666 | 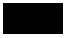 | 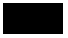 | 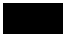 | 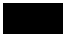 | − | − | − | − | +/− | − | − |
| inlF | 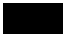 | 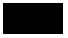 | 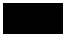 | 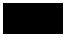 | − | − | − | − | − | − | − |
| Lmo320 | 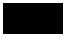 | 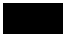 | 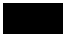 | 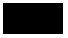 | − | −* | − | − | − | − | − |
| Lmo0842 | 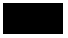 | 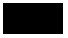 | 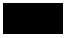 | 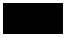 | − | −* | +* | −* | − | + | − |
| Lmo0801 | 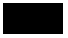 | 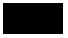 | −* | −* | − | −* | −* | − | +/− | +/− | − |
| inlG | 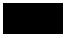 | 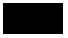 | −* | − | 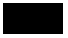 | − | − | − | − | − | − |
| Lmo1115 | 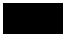 | 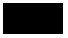 | −* | −* | 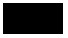 | −* | −* | −* | +/− | − | − |
| Lmo2576 | 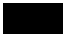 | 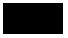 | − | − | 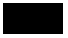 | − | − | − | − | − | − |
| Lmo0171 | 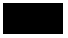 | 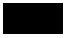 | − | − | − | − | − | − | − | − | − |
| Lmo2026 | 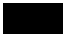 | 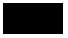 | − | − | − | − | − | − | − | − | − |
| Lmo2178 | + | + | + | + | + | + | + | −* | − | + | − |
| Lmo1799 | + | + | + | + | + | + | +* | + | +/− | + | − |
| Lmo1289 | + | + | + | + | + | + | +* | +* | + | +/− | − |
| Lmo1136 | + | + | + | + | + | + | + | + | + | + | + |
| Lmo0880 | + | + | + | + | + | + | +* | +* | + | + | − |
| Lmo0514 | + | + | + | + | + | + | +* | +* | + | + | − |
| Lmo0175 | + | + | + | + | + | + | + | − | − | − | − |
| Lmo0732 | + | + | +* | +* | + | + | +* | +/− | − | +/− | − |
| Lmo0610 | + | + | + | +* | + | +* | +/− | −* | − | − | − |
| Lin0739 | − | − | − | − | − | − | 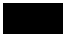 | − | − | − | − |
| Lin0803 | − | − | − | − | − | − | 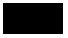 | − | − | − | − |
| Lin0559 | −* | − | −* | −* | − | − | + | − | − | − | − |
| Lin2724 | − | − | − | − | +/− | + | + | −* | − | − | − |
| Lin1204 | − | − | + | +* | − | + | +* | − | − | − | − |
| Lin0372 | +* | +* | + | + | + | + | + | − | − | − | − |
| Lin0415 | +* | +* | +* | +* | + | − | + | − | +/− | + | − |
| Lin0665 | +* | +* | + | + | + | + | + | − | + | +/− | − |
| Lin0740 | +* | +* | +* | +* | + | +* | + | − | − | − | − |
| Lin0661 | +* | +* | −* | −* | + | − | + | − | − | − | − |
| Lin1328 | +* | +* | +* | +* | + | +* | + | +* | +/− | − | − |
| ORF2568 | − | − | 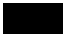 | 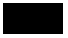 | − | − | − | +/− | − | − | − |
| ORF1761 | − | − | 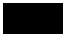 | 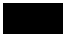 | − | − | − | − | − | − | − |
| ORF0029 | − | − | 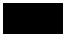 | 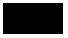 | − | − | − | − | − | − | − |
| ORF1590 | + | − | + | +* | + | − | − | − | − | − | − |
| ORF2541 | −* | − | + | + | − | − | − | − | − | − | − |
| ORF2013 | −* | − | + | +* | + | − | − | − | − | − | − |
| ORF2017 | +* | − | + | + | + | − | −* | +/− | − | − | − |

+ or − : indicates gene presence or absence in all strains of the corresponding serovar
+* or −* : indicates gene presence or absence in at least two thirds of the strains of the corresponding serovar.
+/− was used when the gene is present in one of two strains studied;
Lmo and Lin numbers are corresponding to gene names on the ListiList web server http://genolist.pasteur.fr/ListiList/.
Serovar and total number of strains studied for each lineage are indicated between brackets. Shaded boxes indicate species or lineage specific genes.

TABLE 12

Product size

| | |
|---|---|
| Gene | Accession |

| | | |
|---|---|---|
| targets | Primer sequence (5′-3′) | (bp) |

| | | |
|---|---|---|
| Lmo0737 | For: AGGGCTTCAAGGACTTACCC | 691 |
| | [SEQ ID NO:1477] | |
| | Rev: ACGATTTCTGCTTGCCATTC | |
| | [SEQ ID NO:1478] | |
| Lmo1119 | For: ATCCTGGTGGTAAACATAAAATG | 782 |
| | [SEQ ID NO:1479] | |
| | Rev: | |
| | TGAGAAGAATAAAAATTCAGCACCT | |
| | [SEQ ID NO:1480] | |
| ORF2819 | For: AGCAAAATGCCAAAACTCGT | 471 |
| | [SEQ ID NO:1481] | |
| | Rev: CATCACTAAAGCCTCCCATTG | |
| | [SEQ ID NO:1482] | |
| ORF2110 | For: AGTGGACAATTGATTGGTGAA | 600 |
| | [SEQ ID NO:1483] | |
| | Rev: CATCCATCCCTTACTTTGGAC | |
| | [SEQ ID NO:1484] | |
| prs | For: GCTGAAGAGATTGCGAAAGAAG | 348 |
| | [SEQ ID NO:1485] | |
| | Rev: CAAAGAAACCTTGGATTTGCGG | |
| | [SEQ ID NO:1486] | |

TABLE 13

| | | Total | | | | | | | |
|---|---|---|---|---|---|---|---|---|---|
| | | number | | | | | | Multiplex PCR | Percentage |
| Conventional | | of | Multiplex PCR results | | | | | serovar | of |
| serotyping | Origin | strains | 348pb | 691 bp | 471 pb | 782 pb | 600 pb | interpretation | agreements |
| 1/2a | Human | 20 | + | + | − | − | − | 1/2a or 3a | 100 |
| | Food | 20 | + | + | − | − | − | | |
| 1/2b | Human | 20 | + | − | + | − | − | 1/2b, 3b or 7 | 100 |
| | Food | 20 | + | − | + | − | − | | |
| 1/2c | Human | 20 | + | + | − | + | − | 1/2c or 3c | 100 |
| | Food | 20 | + | + | − | + | − | | |
| 4b | Human | 20 | + | − | + | − | + | 4b, 4d or 4e | 100 |
| | Food | 20 | + | − | + | − | + | | |

TABLE 14

Oligonucleotide primers for L. monocytogenes
specific marker genes

| | | |
|---|---|---|
| Gene | Forward (5′ to 3′) | Reverse (5′ to 3′) |

| | | |
|---|---|---|
| I | | |
| Lmo 0082 | CCAACAGGAGCTAACATGAA | TGGTTAGGAAGATTGGGATT |
| | [SEQ ID NO:1487] | [SEQ ID NO:1488] |
| Lmo 0083 | GAAGGCTTAATAACCCCTGAC | CGCAACCTTCTCCTCATAAAT |
| | C [SEQ ID NO:1489] | C [SEQ ID NO:1490] |
| plcA | CTGACACAACAAACCTAGCAG | TGAATTTTGTTTTCACACTCG |
| | C [SEQ ID NO:1491] | G [SEQ ID NO:1492] |
| actA | GAGGTAAATGCTTCGGACTT | AGGCGATCAATTTCTTCTTC |
| | [SEQ ID NO:1493] | [SEQ ID NO:1494] |
| plcB | GTATGTGCTTGATCGCAAGTG | AAAATTATTGGCGTGCATAGG |
| | T [SEQ ID NO:1495] | T [SEQ ID NO:1496] |
| Lmo206 | TTAGTAATCGCGTTATGTTCC | AGCAACTTTTTGTTCGTCTTC |
| | G [SEQ ID NO:1497] | A [SEQ ID NO:1498] |
| inlA | TAACATCAGTCCCCTAGCAGG | TCCAATAGTGACAGGTTGGCT |
| | T [SEQ ID NO:1499] | A [SEQ ID NO:1500] |
| inlB | AAAAGCAAGATTTCATGGGAG | TCTCACTCTTGCATAAGCGGT |
| | A [SEQ ID NO:1501] | A [SEQ ID NO:1502] |
| Lmo 0440 | TGACAAACAGAACATTAACTA | TTTGAAATCGAATGTTAAATC |
| | CCAA [SEQ ID NO:1503] | CA [SEQ ID NO:1504] |
| Lmo 0638 | TGCTTGGTTTAACGGATTATC | GGCTGTTTGTAATTTATTCGT |
| | A [SEQ ID NO:1505] | GC [SEQ ID NO:1506] |
| Lmo 0733 | GCAAGAAGAAATTGCCATCAA | CCTCCTATAAGGATAATCGGT |
| | [SEQ ID NO:1507] | GC [SEQ ID NO:1508] |
| Lmo 0833 | AATAGGGGCTATTCTTTAGCG | AAAAGCACAGTCATGCGTAGA |
| | G [SEQ ID NO:1509] | A [SEQ ID NO:1510] |
| Lmo 1133 | GACGTGGCTTATTCAGACACA | GCGAACTACTTACCACTTCGC |
| | A [SEQ ID NO:1511] | T [SEQ ID NO:1512] |
| Lmo 1135 | TTAATTGTGGTGCCTGTGTCT | AAAATCCCAGCCTATTCCATC |
| | C [SEQ ID NO:1513] | T [SEQ ID NO:1514] |
| Lmo 2732 | TAAAGCTTTCCGGTTTCTCTG | GGGCATGATGATGTTTAAATG |
| | A [SEQ ID NO:1515] | A [SEQ ID NO:1516] |
| Lmo 2733 | GAATGCCTTTTATCCAAAGAC | AAATCCGGCAATGATACCTAA |
| | G [SEQ ID NO:1517] | A [SEQ ID NO:1518] |
| Lmo 2734 | AACAAGCGCACTTAGAATGAC | CATTTTCGAAGCGAATTAGGT |
| | A [SEQ ID NO:1519] | C [SEQ ID NO:1520] |
| Lmo 2736 | GCCAACACGATTGGATTAGATT | TCGGCGTCTTTCATTTTATCT |
| | [SEQ ID NO:1521] | T [SEQ ID NO:1522] |
| II | | |
| Lmo0017 | TCTTAAAAATGCAGGGGTTGA | GCCGAATGACCTGAAACATAG |
| | T [SEQ ID NO:1523] | T [SEQ ID NO:1524] |
| Lmo0094 | TGATTATTGGTTTATTGATTG | ACTCGCCACAAACAAACTACA |
| | GCTT [SEQ ID NO:1525] | A [SEQ ID NO:1526] |
| inlH | GGGTTACAAAGCATAAAAACG | ATGTTAAATTTGGGCTTGCGT |
| | C [SEQ ID NO:1527] | A [SEQ ID NO:1528] |
| inlE | CGATGTTTCTCCACTTACTGG | AGCAGGCATTTTATCTATCCC |
| | TT [SEQ ID NO:1529] | A [SEQ ID NO:1530] |
| Lmo0333 | AATCCGGTTATTTGGTATGAC | GCAACTGTATAATCTCCGCCT |
| | G [SEQ ID NO:1531] | T [SEQ ID NO:1532] |
| Lmo0549 | TTTATCCCTTTTTCCAAAGCT | TTGTAAGACACAACTTGGGGT |
| | G [SEQ ID NO:1533] | G [SEQ ID NO:1534] |
| Lmo0832 | TGATTTTGGTGTCTCAATAGC | CAATGCTCGTCTCCATGTTAA |
| | A [SEQ ID NO:1535] | A [SEQ ID NO:1536] |
| Lmo0834 | TGAATATGACACAAAAAGTGG | TGAATATGACACAAAAAGTGG |
| | TGAT [SEQ ID NO:1537] | TGAT [SEQ ID NO:1538] |
| Lmo0835 | CATTAGCATAGATGGCTTGC | CGAGCTATCACCTGTTTTTG |
| | [SEQ ID NO:1539] | [SEQ ID NO:1540] |
| Lmo1441 | ATGTCGAAGCAACAGTAGCA | AATTCCGAGATGCAGAGGAT |
| | GA [SEQ ID NO:1541] | AA [SEQ ID NO:1542] |
| Lmo1451 | AGTAGATGGCGCTAATCGAG | CTTGGGCTAAACGAGCAGTAT |
| | AA [SEQ ID NO:1543] | T [SEQ ID NO:1544] |

TABLE 15

Oligonucleotide primers for L. monocytogenes
subgroup specific marker genes

| | | |
|---|---|---|
| Gene | Forward (5′ to 3′) | Reverse (5′ to 3′) |

| | | |
|---|---|---|
| A | | |
| Lmo0171 | AACGTATGAAGCGACGATAA | AAAACCCCACTTGAATTAGC |
| | [SEQ ID NO:1545] | [SEQ ID NO:1546] |
| Lmo0172 | CCGCATTAATGAAACAAGAA | TGGTAGAATGTTTCGCACTT |
| | [SEQ ID NO:1547] | [SEQ ID NO:1548] |
| Lmo0525 | AGGGATTATTATTGGCCGACT | TGTGTCTGTCCATGTCATCAA |
| | T [SEQ ID NO:1549] | A [SEQ ID NO:1550] |
| Lmo0734 | CACTACCAAGGAGCTTTTGA | ATTCAACGGGAACAATCTCT |
| | [SEQ ID NO:1551] | [SEQ ID NO:1552] |
| Lmo0735 | GAAGCGATGTTTTTCATGTTG | TAGCACAAACCCCTTAACACC |
| | A [SEQ ID NO:1553] | T [SEQ ID NO:1554] |
| Lmo0736 | TGGAAATGATCATGTTGGAAT | AAATTCTGCATCCAACCAGTT |
| | C [SEQ ID NO:1555] | T [SEQ ID NO:1556] |
| Lmo0737 | ATTATTAGCAGCATTCGGATC | AATGTCATTGCGAGTGTACCT |
| | G [SEQ ID NO:1557] | G [SEQ ID NO:1558] |
| Lmo0738 | ATTCTTAGCGTTTGGACCTC | AAAGCCAACAAATCCTTTTC |
| | [SEQ ID NO:1559] | [SEQ ID NO:1560] |
| Lmo0739 | GGTCGAGTGAAATATTGGGTT | TTGTTTCACAATATGGGTTTG |
| | C [SEQ ID NO:1561] | G [SEQ ID NO:1562] |
| Lmo1060 | TAACCGAAGCAGGGTATCAAG | TTGCGTAGATTTTTCATATGC |
| | T [SEQ ID NO:1563] | G [SEQ ID NO:1564] |
| Lmo1061 | ATTAAAATCCCCTCTTGCTG | TGATTTCTCAAGGCGATAGA |
| | [SEQ ID NO:1565] | [SEQ ID NO:1566] |
| Lmo1062 | AAACCCCAAGTTTTTCAGTCA | CATTTCAAATTTACGCTCACG |
| | A [SEQ ID NO:1567] | A [SEQ ID NO:1568] |
| Lmo1063 | GGGAAACAAAGCTCAAATTGT | AATAATGTGTTCCGAGGTTTC |
| | T [SEQ ID NO:1569] | A [SEQ ID NO:1570] |
| Lmo1968 | GTGCAATCAGCCAACAAAATA | TTCAAATTCACAGGCATGAAT |
| | A [SEQ ID NO:1571] | C [SEQ ID NO:1572] |
| Lmo1969 | ATGGCAATTATTCGGGATGTA | TTGATTGAGCATAGCCTTTGT |
| | G [SEQ ID NO:1573] | G [SEQ ID NO:1574] |
| Lmo1970 | AGTAACACGAGCTGTTGCAT | AAATCACACGCACTCCTTTA |
| | [SEQ ID NO:1575] | [SEQ ID NO:1576] |
| Lmo1971 | ACAGGAAATGATCAAGTCGGT | AATAATGGCTCCTCGTCTACC |
| | T [SEQ ID NO:1577] | A [SEQ ID NO:1578] |
| Lmo1973 | GGAGGTAGAAAGTCCGGTAG | TCCAACTGTAAAGCCATGTT |
| | [SEQ ID NO:1579] | [SEQ ID NO:1580] |
| Lmo1974 | TGACAGTTCGAAAAGCGATAG | ATAGGAAGTACGTCGCACATG |
| | A [SEQ ID NO:1581] | A [SEQ ID NO:1582] |
| bvrC | TGAAAACAAGGGTTTTTGGAG | TGCTTCGTAAGCTAATTCCCA |
| | T [SEQ ID NO:1583] | T [SEQ ID NO:1584] |
| bvrB | TTTTAGGCTACACGAGTGCAA | CTTGCTGTAAATGACATTGGG |
| | A [SEQ ID NO:1585] | A [SEQ ID NO:1586] |
| B | | |
| Lmo0151 | TGTTGAATAAAACTAGAGGCG | AGCTACTTTATCTTTAAACTT |
| | AAA [SEQ ID NO:1587] | CGCA [SEQ ID NO:1588] |
| Lmo0466 | GCAAAAGGAAAAATCCAAGAG | ACATGCTTTTTCATGAATTGG |
| | A [SEQ ID NO:1589] | A [SEQ ID NO:1590] |
| Lmo0467 | GCGAATATTGCACTTGTTGAG | TACGTCCAAAAGGGTCTACGT |
| | A [SEQ ID NO:1591] | T [SEQ ID NO:1592] |
| Lmo0469 | AATAATTGCGGATTTGGAGAA | GGCCAAGTGAATGACCATAGA |
| | A [SEQ ID NO:1593] | [SEQ ID NO:1594] |
| Lmo0470 | CAAACACCGTTTGATAAAGTG | TGAAGACTCGTAATGACCCAC |
| | C [SEQ ID NO:1595] | A [SEQ ID NO:1596] |
| Lmo0471 | GGAAGGAAAGGTATTAATCGG | TTTTTGAAGGATATTGTGCTC |
| | G [SEQ ID NO:1597] | G [SEQ ID NO:1598] |
| Lmo1118 | AGGGGTCTTAAATCCTGGAAT | GAAGTTTTCCGCTCCCTATTT |
| | A [SEQ ID NO:1599] | T [SEQ ID NO:1600] |
| Lmo1119 | GGTTCTGGTCTTGCCTTAGAA | TCTCTGGCGCTAAATCATAAG |
| | CT [SEQ ID NO:1601] | T [SEQ ID NO:1602] |
| C | | |
| ORF2819 | AAATTTTTCAAGGAGGAGCAA | CAATGTCTCTTCTTTCGCTTT |
| | [SEQ ID NO:1603] | T [SEQ ID NO:1604] |
| ORF3840 | TCAAATACTTGTCCGCATTTC | GCATATTGATAGTGCGTACCG |
| | [SEQ ID NO:1605] | [SEQ ID NO:1606] |
| ORF2568 | GAACGGTCGGTATTTCATTCT | AACCGTTTGTGCCTCTTTACT |
| | [SEQ ID NO:1607] | [SEQ ID NO:1608] |
| ORF1761 | ACGCGAAAGATAATTTTGACC | CCTTAGAGCAAATCCCCATAG |
| | [SEQ ID NO:1609] | [SEQ ID NO:1610] |
| ORF0029 | CCCAGTGAAAAGTGAAAAAGT | CCCAGTGAAAAGTGAAAAAGT |
| | [SEQ ID NO:1611] | [SEQ ID NO:1612] |
| D | | |
| ORF0799 | AAATTCCAAAAGGACGTGAGT | CATTGTTTTTCAACCGTTCAT |
| | [SEQ ID NO:1613] | [SEQ ID NO:1614] |
| ORF2372 | AGATAGCCTGATGCGAGTTTT | ACGTTTTAGGATCTCCACCTG |
| | [SEQ ID NO:1615] | [SEQ ID NO:1616] |
| ORF2110 | AGTGGACAATTGATTGGTGAA | CATCCATCCCTTACTTTGGAC |
| | [SEQ ID NO:1617] | [SEQ ID NO:1618] |

TABLE 16
a: Example of 192 Genes Representative of the 409 Gene Macroarray

| | | | | | | | | | L. mono | | | |
|---|---|---|---|---|---|---|---|---|---|---|---|---|
| | L. ivanovii | L. Welshimeri | L. seeligeri | L. innocua | L. gravi-murravi | L. mono1/2a-3a | L. mono1/2c-3c | L. mono1/2b-3b-7 | 4b-4d-4e | L. mono 4c | L. mono 4a | Gene |
| Lmo0017 | 0 | 0 | 0 | 0 | 0 | 100 | 100 | 100 | 100 | 100 | 0 | Lmo0017 |
| Lmo0036 | 100 | 0 | 50 | 0 | 100 | 100 | 100 | 100 | 100 | 0 | 0 | Lmo0036 |
| Lmo0037 | 100 | 0 | 50 | 0 | 0 | 100 | 100 | 100 | 100 | 0 | 0 | Lmo0037 |
| Lmo0066 | 50 | 0 | 0 | 25 | 0 | 22 | 100 | 15 | 7 | 100 | 33 | Lmo0066 |
| Lmo0067 | 0 | 0 | 0 | 25 | 0 | 7 | 100 | 10 | 0 | 0 | 0 | Lmo0067 |
| Lmo0068 | 0 | 0 | 0 | 25 | 0 | 7 | 100 | 45 | 59 | 0 | 0 | Lmo0068 |
| Lmo0069 | 0 | 0 | 0 | 0 | 0 | 19 | 100 | 80 | 59 | 0 | 0 | Lmo0069 |
| Lmo0072 | 0 | 0 | 0 | 25 | 0 | 41 | 100 | 90 | 100 | 0 | 0 | Lmo0072 |
| Lmo0073 | 0 | 0 | 0 | 0 | 0 | 11 | 100 | 50 | 59 | 0 | 0 | Lmo0073 |
| Lmo0079 | 33 | 0 | 50 | 0 | 0 | 67 | 92 | 100 | 81 | 100 | 0 | Lmo0079 |
| Lmo0080 | 0 | 0 | 50 | 0 | 0 | 48 | 100 | 95 | 85 | 0 | 0 | Lmo0080 |
| Lmo0081 | 0 | 0 | 0 | 0 | 0 | 15 | 92 | 35 | 0 | 0 | 0 | Lmo0081 |
| Lmo0082 | 0 | 0 | 0 | 0 | 0 | 100 | 100 | 100 | 100 | 100 | 100 | Lmo0082 |
| Lmo0083 | 0 | 0 | 0 | 0 | 0 | 100 | 100 | 100 | 100 | 100 | 100 | Lmo0083 |
| Lmo0094 | 0 | 0 | 0 | 0 | 0 | 100 | 100 | 100 | 100 | 0 | 0 | Lmo0094 |
| Lmo0106 | 33 | 50 | 100 | 0 | 0 | 96 | 100 | 35 | 96 | 100 | 100 | Lmo0106 |
| Lmo0141 | 0 | 0 | 0 | 0 | 0 | 30 | 100 | 25 | 96 | 50 | 100 | Lmo0141 |
| Lmo0143 | 0 | 0 | 0 | 0 | 0 | 22 | 100 | 5 | 7 | 0 | 100 | Lmo0143 |
| Lmo0144 | 0 | 0 | 0 | 0 | 0 | 11 | 100 | 25 | 96 | 50 | 100 | Lmo0144 |
| Lmo0147 | 83 | 50 | 50 | 63 | 0 | 100 | 100 | 65 | 85 | 100 | 67 | Lmo0147 |
| Lmo0151 | 0 | 0 | 0 | 0 | 0 | 11 | 100 | 0 | 0 | 0 | 0 | Lmo0151 |
| Lmo0171 | 0 | 0 | 0 | 0 | 0 | 100 | 100 | 0 | 0 | 0 | 0 | Lmo0171 |
| Lmo0172 | 0 | 0 | 0 | 0 | 0 | 100 | 100 | 0 | 0 | 0 | 0 | Lmo0172 |
| Lmo0252 | 0 | 100 | 50 | 0 | 0 | 44 | 100 | 0 | 0 | 100 | 100 | Lmo0252 |
| Lmo0255 | 0 | 0 | 0 | 0 | 0 | 93 | 100 | 30 | 22 | 100 | 100 | Lmo0255 |
| Lmo0304 | 0 | 50 | 0 | 50 | 0 | 56 | 100 | 90 | 7 | 50 | 0 | Lmo0304 |
| Lmo0310 | 0 | 0 | 0 | 0 | 0 | 7 | 100 | 30 | 4 | 0 | 0 | Lmo0310 |
| Lmo0320 | 0 | 0 | 0 | 13 | 0 | 85 | 100 | 90 | 100 | 0 | 33 | Lmo0320 |
| Lmo0333 | 0 | 0 | 0 | 0 | 0 | 100 | 100 | 100 | 100 | 100 | 0 | Lmo0333 |
| Lmo0334 | 0 | 50 | 0 | 0 | 0 | 100 | 100 | 100 | 100 | 100 | 0 | Lmo0334 |
| Lmo0338 | 33 | 0 | 0 | 13 | 50 | 67 | 100 | 0 | 0 | 0 | 0 | Lmo0338 |
| Lmo0378 | 0 | 0 | 0 | 50 | 0 | 100 | 92 | 75 | 19 | 0 | 0 | Lmo0378 |
| Lmo0379 | 0 | 0 | 0 | 25 | 0 | 85 | 100 | 0 | 0 | 100 | 0 | Lmo0379 |
| Lmo0380 | 0 | 0 | 0 | 25 | 0 | 96 | 92 | 75 | 19 | 100 | 0 | Lmo0380 |
| Lmo0381 | 0 | 0 | 0 | 25 | 0 | 100 | 100 | 75 | 19 | 0 | 100 | Lmo0381 |
| Lmo0409 | 0 | 0 | 0 | 0 | 0 | 78 | 100 | 60 | 78 | 0 | 0 | Lmo0409 |
| Lmo0419 | 0 | 100 | 0 | 0 | 0 | 100 | 100 | 100 | 100 | 100 | 0 | Lmo0419 |
| Lmo0440 | 0 | 0 | 0 | 0 | 0 | 100 | 100 | 100 | 100 | 100 | 100 | Lmo0440 |
| Lmo0444 | 0 | 100 | 0 | 0 | 0 | 37 | 100 | 65 | 0 | 100 | 0 | Lmo0444 |
| Lmo0448 | 0 | 100 | 0 | 63 | 0 | 44 | 100 | 95 | 63 | 100 | 33 | Lmo0448 |
| Lmo0459 | 0 | 0 | 0 | 0 | 0 | 48 | 100 | 100 | 100 | 50 | 100 | Lmo0459 |
| Lmo0462 | 0 | 0 | 0 | 0 | 0 | 48 | 92 | 100 | 100 | 50 | 100 | Lmo0462 |
| Lmo0466 | 0 | 0 | 0 | 0 | 0 | 11 | 100 | 0 | 0 | 0 | 0 | Lmo0466 |
| Lmo0467 | 0 | 0 | 0 | 0 | 0 | 11 | 100 | 0 | 0 | 0 | 0 | Lmo0467 |
| Lmo0471 | 0 | 0 | 0 | 0 | 0 | 11 | 100 | 0 | 0 | 0 | 0 | Lmo0471 |
| Lmo0472 | 0 | 50 | 0 | 88 | 0 | 81 | 100 | 25 | 4 | 100 | 0 | Lmo0472 |
| Lmo0473 | 67 | 50 | 0 | 38 | 0 | 93 | 100 | 20 | 67 | 100 | 100 | Lmo0473 |
| Lmo0474 | 0 | 0 | 0 | 25 | 0 | 93 | 100 | 10 | 100 | 0 | 0 | Lmo0474 |
| Lmo0493 | 67 | 0 | 100 | 0 | 0 | 85 | 100 | 100 | 100 | 50 | 0 | Lmo0493 |
| Lmo0497 | 0 | 0 | 0 | 0 | 0 | 100 | 100 | 100 | 4 | 0 | 0 | Lmo0497 |
| Lmo0525 | 0 | 50 | 0 | 0 | 0 | 100 | 100 | 0 | 0 | 0 | 0 | Lmo0525 |
| Lmo0549 | 0 | 0 | 0 | 0 | 0 | 100 | 100 | 100 | 100 | 100 | 0 | Lmo0549 |
| Lmo0630 | 50 | 100 | 0 | 0 | 0 | 100 | 100 | 95 | 81 | 50 | 0 | Lmo0630 |
| Lmo631 | 0 | 100 | 0 | 0 | 0 | 100 | 100 | 100 | 81 | 50 | 0 | Lmo0631 |
| Lmo0632 | 100 | 100 | 0 | 0 | 0 | 100 | 100 | 100 | 81 | 50 | 0 | Lmo0632 |
| Lmo0732 | 50 | 50 | 0 | 75 | 0 | 100 | 100 | 80 | 70 | 100 | 100 | Lmo0732 |
| Lmo0733 | 0 | 0 | 0 | 0 | 0 | 100 | 100 | 100 | 100 | 100 | 100 | Lmo0733 |
| Lmo0734 | 0 | 100 | 0 | 0 | 0 | 100 | 100 | 0 | 0 | 0 | 0 | Lmo0734 |
| Lmo0735 | 17 | 50 | 0 | 0 | 0 | 100 | 100 | 0 | 0 | 0 | 0 | Lmo0735 |
| Lmo0739 | 0 | 0 | 0 | 0 | 0 | 100 | 100 | 0 | 0 | 0 | 0 | Lmo0739 |
| Lmo0750 | 0 | 0 | 0 | 0 | 0 | 100 | 100 | 0 | 4 | 0 | 100 | Lmo0750 |
| Lmo0751 | 17 | 0 | 0 | 0 | 0 | 96 | 100 | 60 | 81 | 0 | 100 | Lmo0751 |
| Lmo0752 | 100 | 0 | 0 | 0 | 0 | 100 | 100 | 100 | 100 | 0 | 100 | Lmo0752 |
| Lmo0753 | 100 | 0 | 0 | 0 | 0 | 100 | 100 | 100 | 100 | 0 | 100 | Lmo0753 |
| Lmo0780 | 33 | 50 | 0 | 0 | 0 | 100 | 100 | 15 | 7 | 100 | 100 | Lmo0780 |
| Lmo0801 | 0 | 50 | 50 | 13 | 0 | 70 | 100 | 0 | 30 | 0 | 33 | Lmo0801 |
| Lmo0804 | 0 | 0 | 0 | 0 | 0 | 44 | 100 | 45 | 33 | 100 | 100 | Lmo0804 |
| Lmo0832 | 0 | 0 | 0 | 0 | 0 | 100 | 100 | 100 | 100 | 0 | 0 | Lmo0832 |
| Lmo0833 | 0 | 0 | 0 | 0 | 0 | 100 | 100 | 100 | 100 | 100 | 33 | Lmo0833 |
| Lmo0834 | 0 | 0 | 0 | 0 | 0 | 100 | 100 | 100 | 100 | 100 | 0 | Lmo0834 |
| Lmo0835 | 0 | 0 | 0 | 0 | 0 | 100 | 100 | 100 | 100 | 0 | 0 | Lmo0835 |
| Lmo0842 | 17 | 100 | 0 | 75 | 0 | 100 | 100 | 95 | 48 | 0 | 33 | Lmo0842 |
| Lmo1030 | 100 | 0 | 0 | 0 | 0 | 100 | 100 | 100 | 100 | 100 | 0 | Lmo1030 |
| Lmo1036 | 100 | 0 | 0 | 0 | 0 | 100 | 100 | 100 | 100 | 100 | 0 | Lmo1036 |
| Lmo1060 | 0 | 100 | 0 | 0 | 0 | 100 | 100 | 0 | 0 | 0 | 0 | Lmo1060 |
| Lmo1061 | 0 | 50 | 0 | 0 | 0 | 100 | 100 | 0 | 0 | 0 | 0 | Lmo1061 |
| Lmo1063 | 0 | 100 | 0 | 0 | 0 | 100 | 100 | 0 | 0 | 0 | 0 | Lmo1063 |
| Lmo1076 | 0 | 0 | 100 | 0 | 0 | 100 | 100 | 100 | 0 | 0 | 0 | Lmo1076 |
| Lmo1077 | 0 | 0 | 100 | 0 | 100 | 100 | 100 | 100 | 0 | 0 | 0 | Lmo1077 |
| Lmo1080 | 0 | 0 | 50 | 0 | 0 | 100 | 100 | 100 | 0 | 0 | 0 | Lmo1080 |
| Lmo1081 | 0 | 0 | 100 | 0 | 0 | 100 | 100 | 100 | 0 | 0 | 0 | Lmo1081 |
| Lmo1082 | 0 | 0 | 100 | 0 | 0 | 100 | 100 | 100 | 0 | 0 | 0 | Lmo1082 |
| Lmo1085 | 0 | 0 | 0 | 0 | 0 | 100 | 100 | 100 | 0 | 0 | 0 | Lmo1085 |
| Lmo1088 | 0 | 0 | 50 | 0 | 0 | 100 | 100 | 100 | 0 | 0 | 0 | Lmo1088 |
| Lmo1090 | 0 | 0 | 100 | 0 | 0 | 100 | 100 | 100 | 0 | 50 | 0 | Lmo1090 |
| Lmo1091 | 0 | 0 | 100 | 0 | 0 | 100 | 100 | 100 | 0 | 0 | 0 | Lmo1091 |
| Lmo1102 | 100 | 0 | 50 | 13 | 0 | 78 | 100 | 65 | 78 | 100 | 100 | Lmo1102 |
| Lmo1115 | 33 | 0 | 50 | 13 | 0 | 78 | 75 | 10 | 4 | 100 | 33 | Lmo1115 |
| Lmo1116 | 67 | 0 | 0 | 63 | 0 | 48 | 100 | 60 | 81 | 50 | 0 | Lmo1116 |
| Lmo1118 | 17 | 0 | 0 | 0 | 0 | 4 | 100 | 0 | 0 | 0 | 0 | Lmo1118 |
| Lmo1119 | 0 | 0 | 0 | 0 | 0 | 4 | 100 | 0 | 0 | 0 | 0 | Lmo1119 |
| Lmo1120 | 67 | 50 | 50 | 25 | 0 | 85 | 100 | 95 | 85 | 0 | 0 | Lmo1120 |
| Lmo1133 | 0 | 0 | 0 | 0 | 0 | 100 | 100 | 100 | 100 | 100 | 100 | Lmo1133 |
| Lmo1135 | 0 | 0 | 0 | 0 | 0 | 100 | 100 | 100 | 100 | 100 | 33 | Lmo1135 |
| Lmo1307 | 33 | 50 | 0 | 0 | 0 | 100 | 100 | 0 | 0 | 100 | 100 | Lmo1307 |
| Lmo1441 | 0 | 0 | 0 | 0 | 0 | 100 | 100 | 100 | 100 | 0 | 0 | Lmo1441 |
| Lmo1451 | 0 | 0 | 0 | 0 | 0 | 100 | 100 | 100 | 100 | 0 | 0 | Lmo1451 |
| Lmo1597 | 67 | 100 | 0 | 0 | 0 | 63 | 100 | 85 | 19 | 0 | 0 | Lmo1597 |
| Lmo1604 | 33 | 0 | 0 | 75 | 0 | 100 | 100 | 75 | 67 | 100 | 33 | Lmo1604 |
| Lmo1648 | 17 | 50 | 0 | 25 | 0 | 78 | 100 | 20 | 7 | 50 | 0 | Lmo1648 |
| Lmo1666 | 0 | 0 | 50 | 0 | 0 | 100 | 100 | 100 | 100 | 0 | 0 | Lmo1666 |
| Lmo1714 | 0 | 0 | 0 | 25 | 0 | 41 | 100 | 100 | 100 | 0 | 100 | Lmo1714 |
| Lmo1968 | 0 | 0 | 0 | 13 | 0 | 100 | 100 | 0 | 0 | 0 | 0 | Lmo1968 |
| Lmo1973 | 0 | 0 | 0 | 13 | 0 | 100 | 100 | 0 | 0 | 0 | 0 | Lmo1973 |
| Lmo1974 | 0 | 0 | 0 | 13 | 0 | 100 | 100 | 0 | 0 | 0 | 0 | Lmo1974 |
| Lmo2026 | 0 | 0 | 0 | 0 | 0 | 41 | 100 | 0 | 0 | 0 | 0 | Lmo2026 |
| Lmo2027 | 0 | 0 | 0 | 0 | 0 | 93 | 100 | 75 | 70 | 100 | 0 | Lmo2027 |
| Lmo2276 | 0 | 0 | 0 | 0 | 0 | 78 | 75 | 70 | 67 | 0 | 0 | Lmo2276 |
| Lmo2277 | 17 | 0 | 0 | 25 | 0 | 56 | 42 | 10 | 11 | 50 | 33 | Lmo2277 |
| Lmo2311 | 0 | 0 | 0 | 0 | 0 | 41 | 75 | 5 | 0 | 50 | 100 | Lmo2311 |
| Lmo2312 | 0 | 50 | 0 | 25 | 0 | 56 | 75 | 80 | 33 | 0 | 100 | Lmo2312 |
| Lmo2324 | 0 | 50 | 0 | 38 | 0 | 63 | 83 | 35 | 41 | 0 | 67 | Lmo2324 |
| Lmo2408 | 0 | 100 | 0 | 0 | 0 | 37 | 100 | 0 | 0 | 0 | 0 | Lmo2408 |
| Lmo2443 | 0 | 0 | 0 | 0 | 0 | 89 | 100 | 95 | 100 | 0 | 100 | Lmo2443 |
| Lmo2576 | 0 | 0 | 0 | 0 | 0 | 100 | 100 | 0 | 0 | 100 | 0 | Lmo2576 |
| Lmo2594 | 0 | 0 | 0 | 50 | 0 | 74 | 92 | 80 | 100 | 100 | 33 | Lmo2594 |
| Lmo2595 | 0 | 0 | 0 | 0 | 0 | 52 | 100 | 40 | 100 | 50 | 100 | Lmo2595 |
| Lmo2671 | 17 | 100 | 50 | 0 | 0 | 100 | 100 | 100 | 100 | 0 | 0 | Lmo2671 |
| Lmo2686 | 17 | 0 | 0 | 0 | 0 | 93 | 100 | 5 | 0 | 50 | 0 | Lmo2686 |
| Lmo2732 | 0 | 0 | 0 | 0 | 0 | 100 | 100 | 100 | 100 | 100 | 100 | Lmo2732 |
| Lmo2736 | 0 | 0 | 0 | 0 | 0 | 100 | 100 | 100 | 100 | 100 | 100 | Lmo2736 |
| Lmo2786 | 0 | 0 | 0 | 0 | 0 | 100 | 100 | 0 | 0 | 100 | 0 | Lmo2786 |
| Lmo2787 | 0 | 0 | 0 | 0 | 0 | 100 | 100 | 0 | 0 | 100 | 0 | Lmo2787 |
| Lmo2807 | 17 | 0 | 50 | 13 | 0 | 63 | 100 | 15 | 48 | 0 | 67 | Lmo2807 |
| Lmo2809 | 0 | 0 | 50 | 25 | 0 | 70 | 92 | 100 | 44 | 0 | 0 | Lmo2809 |
| Lmo2821 | 0 | 0 | 0 | 0 | 0 | 100 | 100 | 100 | 100 | 100 | 0 | Lmo2821 |
| Lin0060 | 0 | 0 | 0 | 63 | 0 | 4 | 0 | 15 | 22 | 0 | 0 | Lin0060 |
| Lin0197 | 100 | 100 | 100 | 100 | 0 | 0 | 0 | 0 | 0 | 0 | 0 | Lin0197 |
| Lin0332 | 0 | 0 | 0 | 38 | 0 | 0 | 0 | 0 | 0 | 0 | 0 | Lin0332 |
| Lin0397 | 0 | 0 | 0 | 50 | 0 | 0 | 0 | 0 | 0 | 0 | 0 | Lin0397 |
| Lin0415 | 0 | 100 | 50 | 100 | 0 | 70 | 75 | 70 | 78 | 100 | 0 | Lin0415 |
| Lin0453 | 0 | 100 | 0 | 100 | 0 | 0 | 0 | 0 | 100 | 0 | 0 | Lin0453 |
| Lin0454 | 17 | 100 | 0 | 100 | 0 | 0 | 0 | 0 | 100 | 100 | 100 | Lin0454 |
| Lin0455 | 33 | 50 | 0 | 100 | 0 | 0 | 0 | 0 | 67 | 50 | 100 | Lin0455 |
| Lin0465 | 0 | 0 | 0 | 100 | 0 | 11 | 0 | 0 | 0 | 0 | 0 | Lin0465 |
| Lin0476 | 0 | 0 | 0 | 38 | 0 | 59 | 67 | 40 | 30 | 100 | 0 | Lin0476 |
| Lin0661 | 0 | 0 | 0 | 100 | 0 | 52 | 50 | 5 | 15 | 50 | 0 | Lin0661 |
| Lin0665 | 0 | 50 | 100 | 100 | 0 | 59 | 58 | 100 | 100 | 100 | 100 | Lin0665 |
| Lin0677 | 0 | 0 | 0 | 100 | 0 | 11 | 0 | 0 | 0 | 100 | 100 | Lin0677 |
| Lin0739 | 0 | 0 | 0 | 100 | 0 | 0 | 0 | 0 | 0 | 0 | 0 | Lin0739 |
| Lin0740 | 0 | 0 | 0 | 100 | 0 | 74 | 75 | 40 | 30 | 100 | 67 | Lin0740 |
| Lin0803 | 0 | 0 | 0 | 100 | 0 | 0 | 0 | 0 | 0 | 0 | 0 | Lin0803 |
| Lin0868 | 0 | 0 | 0 | 50 | 0 | 0 | 0 | 0 | 0 | 0 | 0 | Lin0868 |
| Lin1066 | 0 | 0 | 0 | 50 | 0 | 0 | 0 | 0 | 4 | 0 | 0 | Lin1066 |
| Lin1069 | 0 | 0 | 0 | 63 | 0 | 0 | 0 | 0 | 0 | 0 | 0 | Lin1069 |
| Lin1073 | 83 | 50 | 0 | 100 | 0 | 0 | 0 | 0 | 100 | 100 | 100 | Lin1073 |
| Lin1083 | 0 | 0 | 0 | 38 | 0 | 7 | 0 | 35 | 0 | 0 | 0 | Lin1083 |
| Lin1084 | 17 | 50 | 100 | 88 | 0 | 59 | 0 | 100 | 85 | 0 | 0 | Lin1084 |
| Lin1090 | 67 | 0 | 0 | 75 | 0 | 0 | 0 | 100 | 100 | 100 | 67 | Lin1090 |
| Lin1204 | 0 | 0 | 0 | 63 | 0 | 0 | 0 | 90 | 100 | 0 | 100 | Lin1204 |
| Lin1262 | 0 | 0 | 0 | 75 | 0 | 74 | 75 | 50 | 67 | 100 | 67 | Lin1262 |
| Lin1299 | 33 | 0 | 50 | 100 | 0 | 78 | 92 | 70 | 78 | 100 | 67 | Lin1299 |
| Lin1328 | 83 | 0 | 50 | 100 | 0 | 74 | 75 | 50 | 70 | 100 | 67 | Lin1328 |
| Lin1452 | 0 | 0 | 0 | 100 | 0 | 0 | 0 | 5 | 0 | 0 | 0 | Lin1452 |
| Lin1621 | 0 | 0 | 0 | 88 | 0 | 0 | 0 | 0 | 0 | 0 | 100 | Lin1621 |
| Lin2397 | 0 | 50 | 50 | 75 | 0 | 41 | 33 | 30 | 11 | 0 | 0 | Lin2397 |
| Lin2494 | 0 | 100 | 50 | 100 | 0 | 89 | 100 | 95 | 41 | 100 | 100 | Lin2494 |
| Lin2603 | 0 | 0 | 50 | 63 | 0 | 30 | 50 | 30 | 11 | 0 | 100 | Lin2603 |
| Lin2724 | 17 | 0 | 0 | 100 | 0 | 0 | 0 | 0 | 0 | 50 | 100 | Lin2724 |
| Lin2741 | 0 | 0 | 0 | 100 | 0 | 0 | 0 | 0 | 0 | 0 | 0 | Lin2741 |
| Lin2743 | 17 | 50 | 50 | 88 | 0 | 52 | 0 | 60 | 0 | 50 | 0 | Lin2743 |
| Lin2744 | 17 | 50 | 50 | 100 | 0 | 67 | 67 | 75 | 7 | 100 | 0 | Lin2744 |
| Lin2839 | 0 | 0 | 0 | 100 | 0 | 30 | 0 | 25 | 0 | 0 | 0 | Lin2839 |
| Lin2918 | 0 | 0 | 0 | 100 | 0 | 0 | 0 | 0 | 0 | 0 | 0 | Lin2918 |
| ORF 2372 | 33 | 100 | 0 | 0 | 0 | 0 | 0 | 0 | 100 | 0 | 0 | ORF 2372.1 |
| ORF 2110 | 67 | 50 | 0 | 0 | 0 | 0 | 0 | 0 | 100 | 0 | 0 | ORF 2110.1 |
| ORF 799 | 0 | 50 | 0 | 63 | 0 | 7 | 0 | 0 | 100 | 0 | 0 | ORF 799.1 |
| ORF 3840 | 17 | 0 | 0 | 0 | 0 | 0 | 0 | 100 | 100 | 0 | 0 | ORF 3840.1 |
| ORF 2017a | 0 | 0 | 0 | 0 | 0 | 0 | 0 | 100 | 100 | 0 | 0 | ORF 1761.1 |
| ORF 2819 | 0 | 0 | 0 | 0 | 0 | 0 | 0 | 100 | 100 | 0 | 0 | ORF 2819.1 |
| ORF 2541 | 0 | 0 | 0 | 0 | 0 | 30 | 0 | 100 | 100 | 0 | 0 | ORF 2541.1 |
| ORF 2017b | 50 | 0 | 0 | 25 | 0 | 26 | 0 | 100 | 100 | 100 | 33 | ORF 2017.1 |
| ORF 3401 | 33 | 0 | 0 | 25 | 0 | 11 | 0 | 50 | 100 | 0 | 33 | ORF 3401.1 |
| ORF 2074 | 100 | 100 | 50 | 50 | 0 | 48 | 0 | 100 | 100 | 0 | 100 | ORF 2074.1 |
| ORF 2777 | 50 | 50 | 100 | 0 | 0 | 33 | 0 | 100 | 100 | 0 | 0 | ORF 2777.1 |
| ORF 2579 | 100 | 100 | 50 | 38 | 0 | 52 | 0 | 95 | 100 | 0 | 100 | ORF 2579.1 |
| ORF 660 | 0 | 0 | 0 | 0 | 0 | 33 | 0 | 10 | 100 | 0 | 0 | ORF 660.1 |
| ORF 1445 | 50 | 0 | 0 | 0 | 0 | 0 | 0 | 0 | 100 | 100 | 100 | ORF 1445.1 |
| ORF 1590 | 0 | 0 | 0 | 0 | 0 | 100 | 0 | 95 | 100 | 100 | 0 | ORF 1590.1 |
| ORF 1491 | 33 | 100 | 100 | 0 | 0 | 0 | 0 | 100 | 100 | 100 | 0 | ORF 1491.1 |
| ORF 3792 | 17 | 0 | 0 | 0 | 0 | 67 | 17 | 100 | 100 | 100 | 100 | ORF 3792.1 |
| ORF 4101 | 67 | 100 | 0 | 75 | 0 | 59 | 42 | 95 | 100 | 100 | 33 | ORF 4101.1 |
| ORF 1490 | 33 | 100 | 100 | 0 | 100 | 4 | 0 | 100 | 100 | 100 | 0 | ORF 1490.1 |
| ORF 2858 | 50 | 50 | 50 | 50 | 100 | 74 | 75 | 90 | 100 | 100 | 33 | ORF 2858.1 |
| ORF 757 | 0 | 0 | 0 | 38 | 0 | 4 | 0 | 35 | 100 | 0 | 0 | ORF 757.1 |
| ORF 2076 | 100 | 100 | 50 | 50 | 0 | 48 | 0 | 100 | 100 | 0 | 100 | ORF 2076.1 |
| ORF 3184 | 33 | 50 | 0 | 25 | 0 | 26 | 33 | 70 | 96 | 50 | 33 | ORF 3184.1 |
| ORF 2847 | 0 | 0 | 0 | 0 | 0 | 7 | 0 | 0 | 74 | 0 | 100 | ORF 2847.1 |
| ORF 105 | 67 | 0 | 0 | 25 | 0 | 0 | 0 | 75 | 70 | 0 | 0 | ORF 105.1 |
| ORF 1390 | 33 | 0 | 50 | 25 | 0 | 26 | 8 | 15 | 56 | 0 | 67 | ORF 1390.1 |
| ORF 3103 | 0 | 0 | 0 | 25 | 0 | 67 | 0 | 50 | 41 | 0 | 0 | ORF 3103.1 |
| ORF 817 | 33 | 0 | 0 | 13 | 0 | 0 | 0 | 95 | 37 | 50 | 0 | ORF 817.1 |

| | Gene description | Gene function category |
|---|---|---|
| Lmo0017 | 372aa/unknown, similar to Bacillus anthracis CapA | Cell wall |
| | protein (polyglutamate capsule biosynthesis) | |
| Lmo0036 | 341aa/unknown, similar to ornithine | Metabolism of amino acids and |
| | carbamoyltransferase, related molecules | |
| Lmo0037 | 461aa/unknown, similar to amino acid transporter | Transport/binding proteins and |
| | | lipoproteins |
| Lmo0066 | 604aa/unknwon, similar to toxin components | miscellaneous |
| Lmo0067 | 329aa/unknown, similar to dinitrogenase reductase | Protein modification |
| | ADP-ribosylation system | |
| Lmo0068 | 107aa/unknwon | unknown |
| Lmo0069 | 97aa/unknwon | unknown |
| Lmo0072 | 67aa/unknown, Hypothetical | unknown |
| Lmo0073 | 94aa/unknwon | unknown |
| Lmo0079 | 492aa/unknown | unknown |
| Lmo0080 | 132aa/unknwon | unknown |
| Lmo0081 | 200aa/unknwon | unknown |
| Lmo0082 | 108aa/Unknwon | unknown |
| Lmo0083 | 122aa/unknown, similar to transcription regulator | Régulation |
| | (merR family) | |
| Lmo0094 | 186aa/unknwon | unknown |
| Lmo0106 | 300aa/unknown, similar to transcription regulator | Régulation |
| Lmo0141 | 80aa/unknwon | unknown |
| Lmo0143 | 58aa/unknwon | unknown |
| Lmo0144 | 147aa/unknwon | unknown |
| Lmo0147 | 145aa/unknwon | unknown |
| Lmo0151 | 135aa/unknwon | unknown |
| Lmo0171 | 832aa/unknwon, similar to internalin proteins | cell surface proteins |
| | putative peptidoglycan bound protein (LPXTG motif) | |
| Lmo0172 | 112aa/unknown, similar to transposase C-terminal | Transposon and IS |
| | part | |
| Lmo0252 | 128aa/unknown, similar to repressor (penicilinase | Régulation |
| | repressor) | |
| Lmo0255 | 167aa/unknown, similar to unknown protein | unknown |
| Lmo0304 | 421aa/Unknwon | unknown |
| Lmo0310 | 358aa/unknown | unknown |
| Lmo0320 | 399aa/unknown, similar to surface protein | cell surface proteins |
| | (peptidoglycan bound, LPXTG motif) | |
| Lmo0333 | 1778aa/unknown, similar to internalin proteins | cell surface proteins |
| | putative peptidoglycan bound protein (LPXTG motif) | |
| Lmo0334 | 174aa/unknwon | unknown |
| Lmo0338 | 126aa/unknwon | unknown |
| Lmo0378 | 148aa/unknown | unknown |
| Lmo0379 | 151aa/unknwon | unknown |
| Lmo0380 | 209aa/unknwon | unknown |
| Lmo0381 | 128aa/unknwon | unknown |
| Lmo0409 | 821aa/inIF Internalin, putative peptidoglycan bound | cell surface proteins |
| | protein (LPXTG motif) | |
| Lmo0419 | 184aa/unknown, similar to unknown protein | unknown |
| Lmo0440 | 167aa/unknown | unknown |
| Lmo0444 | 926aa/unknown, conserved hypothetical protein | unknown |
| Lmo0448 | 483/unknown, similar to amino acid antiporter | Transport/binding proteins and |
| | | lipoproteins |
| Lmo0459 | 512aa/unknown, similar to transcription regulator | Régulation |
| | (VirR from Streptococcus pyogenes) | |
| Lmo0462 | 161aa/unknwon | unknown |
| Lmo0466 | 567aa/unknown | unknown |
| Lmo0467 | 107aa/unknwon | unknown |
| Lmo0471 | 336aa/unknwon | unknown |
| Lmo0472 | 295aa/unknwon | unknown |
| Lmo0473 | 361aa/unknwon | unknown |
| Lmo0474 | 115aa/unknwon | unknown |
| Lmo0493 | 586aa/unknown, similar to acylase | Detoxification |
| Lmo0497 | 502aa/unknown, similar to sugar transferase | Cell wall |
| Lmo0525 | 443aa/unknwon | unknown |
| Lmo0549 | 673aa/unknown, similar to internalin protein | cell surface proteins |
| Lmo0630 | 676aa/unknown, similar to transcription | Régulation |
| | antiterminator BgIG family | |
| Lmo631 | 150aa/unknown, similar to PTS system, fructose- | Transport/binding proteins and |
| | specific IIA component | lipoproteins |
| Lmo0632 | 353aa/unknown, similar to PTS system, fructose- | Transport/binding proteins and |
| | specific IIC component, lipoproteins | |
| Lmo0732 | 638aa/unknown, putative peptidoglycan bound | cell surface proteins |
| | protein (LPXTG motif) | |
| Lmo0733 | 169aa/unknown, similar to transcription regulator | R+E acu egulation |
| Lmo0734 | 334aa/unknown, similar to transcriptional regulator | Régulation |
| | (Lacl family) | |
| Lmo0735 | 223aa/unknown, similar to Ribulose-5-Phosphate 3- | Specific pathways |
| | Epimerase | |
| Lmo0739 | 457aa/unknown, similar to 6-phospho-beta- | Specific pathways |
| | glucosidase | |
| Lmo0750 | 171aa/unknwon | unknown |
| Lmo0751 | 98aa/unknwon | unknown |
| Lmo0752 | 278aa/unknown, weakly similar to a putative | Detoxification |
| | haloacetate dehalogenase | |
| Lmo0753 | 226aa/unknown, similar to transcription regulator | Régulation |
| | Crp/Fnr family | |
| Lmo0780 | 115aa/unknown | unknown |
| Lmo0801 | 646aa/unknown, similar to internalin, putative | cell surface proteins |
| | peptidoglycan bound protein (LPXTG motif) | |
| Lmo0804 | 156aa/Unknwon | unknown |
| Lmo0832 | 94aa/unknown, similar to transposase | Tranaposon and IS |
| Lmo0833 | 296aa/unknown; similar to transcriptional regulator | Regulation |
| Lmo0834 | 237aa/Unknwon | unknown |
| Lmo0835 | 334aa/unknown, putative peptidoglycan bound | cell surface proteins |
| | protein (LPXTG motif) | |
| Lmo0842 | 2044aa/unknown, surface protein (LPXTG motif) | cell surface proteins |
| Lmo1030 | 342aa/unknown, similar to transcriptional regulator | Régulation |
| | Lacl family | |
| Lmo1036 | 421aa/unknwon | unknown |
| Lmo1060 | 220aa/unknown, similar to transcription response | Régulation |
| | regulator | |
| Lmo1061 | 481aa/unknown, similar to sensor protein histidine | Sensors (signal transduction) |
| | kinases (2 components regulatory systems) | |
| Lmo1063 | 222aa/unknown, similar to ABC transporter (ATP | Transport/binding proteins and |
| | binding protein), lipoproteins | |
| Lmo1076 | 572aa/unknown, similar to AUTOLYSIN (EC 3.5.1.28) | Cell wall |
| | (N-ACETYLMURAMOYL-L-ALANINE AMIDASE) | |
| Lmo1077 | 562aa/unknown, similar to teichoic acid biosynthesis | Cell wall |
| | protein B | |
| Lmo1080 | 623aa/unknown, similar to B. subtilis minor teichoic | Cell wall |
| | acids biosynthesis protein GgaB | |
| Lmo1081 | 288aa/unknown, similar to glucose-1-phosphate | Cell wall |
| | thymidyl transferase | |
| Lmo1082 | 186aa/unknown, similar to dTDP-sugar epimerase | Cell wall |
| Lmo1085 | 696aa/unknown, similar to teichoic acid biosynthesis | Cell wall |
| | protein B | |
| Lmo1088 | 386aa/tagB: unknown, similar to teichoic acid | Cell wall |
| | biosynthesis protein B precursor | |
| Lmo1090 | 327aa/unknown, similar to glycosyltransferases | Cell wall |
| Lmo1091 | 337aa/unknown, siumilar to glysosyltransferases | Cell wall |
| Lmo1102 | 111aa/unknown, similar to cadmium efflux system | Detoxification |
| | accessory proteins | |
| Lmo1115 | 862aa/unknown, similar to fibrinogen-binding protein | cell surface proteins |
| | (LPXTG motif) | |
| Lmo1116 | 257aa/unknown, similar to regulatory proteins | Régulation |
| Lmo1118 | 505aa/unknwon | unknown |
| Lmo1119 | 285aa/unknown, similar to methylases | DNA restriction/modification and |
| | | repair |
| Lmo1120 | 138aa/unknwon | unknown |
| Lmo1133 | 102aa/unknown, similar to B. subtilis YjcS protein | unknown |
| Lmo1135 | 383aa/unknwon | unknown |
| Lmo1307 | 280aa/unknwon | unknown |
| Lmo1441 | 368aa/unknown, similar to putative peptidoglycan | Cell wall |
| | acetylation protein | |
| Lmo1451 | 331aa/unknown, similar to E. coli LytB protein | unknown |
| Lmo1597 | 259aa/unknwon | unknown |
| Lmo1604 | 181aa/unknown | unknown |
| Lmo1648 | 120aa/unknwon | unknown |
| Lmo1666 | 1711aa/unknown, peptidoglycan linked protein | cell surface proteins |
| | (LPxTG) | |
| Lmo1714 | 190aa/unknwon | unknown |
| Lmo1968 | 247aa/unknown, similar to creatinine | Metabolism of amino acids and |
| | amidohydrolases | related molecules |
| Lmo1973 | 147aa/unknown, similar to PTS system enzyme II A | Transport/binding proteins and |
| | component | lipoproteins |
| Lmo1974 | 249aa/unknown, similar to transcription regulators | Régulation |
| | (GntR family) | |
| Lmo2026 | 626aa/unknown, peptidoglycan linked protein | cell surface proteins |
| | (LPxTG) | |
| Lmo2027 | 367aa/unknown, putative cell surface protein, similar | cell surface proteins |
| | to internalin proteins | |
| Lmo2276 | 165aa/unknown, similar to an unknown | Phage-related functions |
| | bacteriophage protein | |
| Lmo2277 | 183aa/unknwon | unknown |
| Lmo2311 | 153aa/unknwon | unknown |
| Lmo2312 | 156aa/unknwon | unknown |
| Lmo2324 | 258aa/unknown, similar to anti-repressor | Régulation |
| | [Bacteriophage A118] | |
| Lmo2408 | 67aa/unknown, similar to repressor protein | Régulation |
| Lmo2443 | 139aa/unknwon | unknown |
| Lmo2576 | 1530aa/unknwo, peptidoglycan anchored protein | cell surface proteins |
| | (LPXTG motif) | |
| Lmo2594 | 119aa/unknwon | unknown |
| Lmo2595 | 289aa/unknown, similar to unknown proteins | unknown |
| Lmo2671 | 122aa/unknwon | unknown |
| Lmo2686 | 177aa/unknwon | unknown |
| Lmo2732 | 250aa/unknwon | unknown |
| Lmo2736 | 377aa/unknown, conserved hypothetical protein | unknown |
| Lmo2786 | 327aa/bvrC | |
| Lmo2787 | 640aa/bvrB-beta-glucoside-specific | Transport/binding proteins and |
| | phosphotransferase enzyme II | lipoproteins |
| Lmo2807 | 127aa/unknown, hypothetical secreted protein | unknown |
| Lmo2809 | 126aa/unknown, hypothetical secreted protein | unknown |
| Lmo2821 | 851aa/unknown, similar to internalin, unknown | Cell surface proteins |
| | putative peptidoglycan bound protein (LPXTG motif) | |
| Lin0060 | 204aa/unknown | unknown |
| Lin0197 | 358aa/unknown, similar to chloromuconate | Metabolism of carbohydrates |
| | cycloisomerase ykfB of B. subtilis | (Specific pathways) |
| Lin0332 | 405aa/unknown, similar to putative permeases | Transport/binding proteins and |
| | | lipoproteins |
| Lin0397 | 120aa/unknwon | unknown |
| Lin0415 | 410aa/unknown, probable cell surface protein | cell surface proteins |
| | (LPXTG motif) | |
| Lin0453 | 169aa/unknown | unknown |
| Lin0454 | 2167aa/unknown, similar to cell wall-associated | cell surface proteins |
| | protein precursor wapA (B. subtilis) | |
| Lin0455 | 184aa/unknwon | unknown |
| Lin0465 | 212aa/unknown, conserved hypothetical protein | unknown |
| | similar to yoaZ B. subtilis | |
| Lin0476 | 350aa/unknwon | unknown |
| Lin0661 | 596aa/unknown, internalin like protein (LPXTG motif) | cell surface proteins |
| Lin0665 | 831aa/unknown, highly similar to ORFA of Listeria | cell surface proteins |
| | seeligeri | (LPXTG motif) |
| Lin0677 | 293aa/unknown, conserved hypothetical protein | unknown |
| Lin0739 | 521aa/unknown, internalin like protein (LPXTG motif) | cell surface proteins |
| Lin0740 | 657aa/unknown, probable cell surface protein | Cell surface proteins |
| | (LPXTG motif) | |
| Lin0803 | 1386aa/unknown, surface protein (LPXTG motif) | cell surface proteins |
| Lin0868 | 378aa/unknwon | unknown |
| Lin1066 | 329aa/unknown, similar to dolichol, phosphate | Cell wall |
| | mannose synthase | |
| Lin1069 | 336aa/unknwon | unknown |
| Lin1073 | 462aa/unknown, similar to galactosamine-containing | Cell wall |
| | minor teichoic acid biosynthesis protein GgaA | |
| Lin1083 | 216aa/unknwon | unknown |
| Lin1084 | 94aa/unknwon | unknown |
| Lin1090 | 111aa/unknwon | unknown |
| Lin1204 | 1237aa/unknown, similar to internalin proteins | Cell surface proteins |
| | (LPXTG motif) | |
| Lin1262 | 445aa/unknwon | unknown |
| Lin1299 | 121aa/unknwon | unknown |
| Lin1328 | 620aa/unknown, internalin like protein (LPXTG motif) | Cell surface proteins |
| Lin1452 | 125aa/unknwon | unknown |
| Lin1621 | 253aa/unknown, similar to ketoacyl reductases | Metabolism of lipids |
| Lin2397 | 190aa/unknown, similar to sigma factor-like positive | RNA synthesis (Initiation) |
| | control protein of B. subtilis | |
| Lin2494 | 124aa/unknown, hypothetical protein | unknown |
| Lin2603 | 95aa/unknwon | unknown |
| Lin2724 | 938aa/unknown, internalin-like protein (LPXTG motif) | cell surface proteins |
| Lin2741 | 152aa/unknwon | unknown |
| Lin2743 | 98aa/unknwon | unknown |
| Lin2744 | 1334aa/unknown, similar to hypothetical proteins | unknown |
| Lin2839 | 99aa/unknwon | unknown |
| Lin2918 | 159aa/unknwon | unknown |
| ORF 2372 | 596pb/unknown, similar ti teichoic acid biosynthesis | cell wall |
| | protein C | |
| ORF 2110 | 597pb/unknown, similar to hypothetical protein | unknown |
| ORF 799 | 453pb/unknown | unknown |
| ORF 3840 | 593pb/unknown, similar to transcription regulator | Régulation |
| ORF 2017a | 599pb/unknwon, similar to internalin proteins | cell surface proteins |
| | putative peptidoglycan bound protein (LPXTG motif) | |
| ORF 2819 | 562pb/unknown, similar to transcriptional regulator | Régulation |
| ORF 2541 | 590pb/unknwon, similar to internalin proteins | cell surface proteins |
| | putative peptidoglycan bound protein (LPXTG motif) | |
| ORF 2017b | 285pb/unknwon, similar to internalin proteins | cell surface proteins |
| | putative peptidoglycan bound protein (LPXTG motif) | |
| ORF 3401 | 575pb/unknown | unknown |
| ORF 2074 | 599pb/unknown similar to oligo-1,6-glucosidase | Specific pathways |
| ORF 2777 | 600pb/unknown | unknown |
| ORF 2579 | 601pb/unknown, sucrose phosphorylase | Specific pathways |
| ORF 660 | 603pb/unknown | unknown |
| ORF 1445 | 607pb/unknown, conserved hypothetical protein | unknown |
| | ykcB - Bacillus subtills | |
| ORF 1590 | 588pb/unknwon, similar to internalin proteins | cell surface proteins |
| | putative peptidoglycan bound protein (LPXTG motif) | |
| ORF 1491 | 598pb/unknown, similar to hypothetical protein | unknown |
| ORF 3792 | 594pb/unknown | unknown |
| ORF 4101 | 613pb/unknown, similar to similar to ABC | Transport/binding proteins and |
| | transporter | lipoproteins |
| ORF 1490 | 474pb/unknown, similar to sucrose | Transport/binding proteins and |
| | phosphotransferase enzyme IIBC component | lipoproteins |
| ORF 2858 | 249pb/unknown, similar to hypothetical protein | unknown |
| ORF 757 | 522pb/unknown | unknown |
| ORF 2076 | 597pb/unknown, similar to maltodextrin ABC | Transport/binding proteins and |
| | transporter | lipoproteins |
| ORF 3184 | 597pb/unknown | unknown |
| ORF 2847 | 590pb/similar to phage integrase | Phage-related functions |
| ORF 105 | 599pb/unknown, similar to similar to ABC | Transport/binding proteins and |
| | transporter | lipoproteins |
| ORF 1390 | 624pb/unknown, similar to hypothetical protein | unknown |
| ORF 3103 | 600pb/unknown, similar to peptidoglycan lytic | cell wall |
| | protein P45 | |
| ORF 817 | 594pb/unknown | unknown |

b:Example of 192 Genes Representative of the 409 Gene Macroarray

| Gene | CLIP42636-1/2aE | CLIP61523-1/2a | CLIP42651-1/2a | CLIP42663-1/2a | CLIP46661-1/2a | CLIP46664-1/2a | CLIP46682-1/2a | CLIP61634-1/2a | CLIP9429-1/2a | CLIP61678-1/2a |
|---|---|---|---|---|---|---|---|---|---|---|
| Lmo0017 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 |
| Lmo0066 | 0 | 0 | 0 | 0 | 0 | 0 | 2 | 0 | 0 | 2 |
| Lmo0067 | 0 | 0 | 0 | 0 | 0 | 0 | 0 | 0 | 0 | 2 |
| Lmo0068 | 0 | 0 | 0 | 0 | 0 | 0 | 0 | 0 | 0 | 2 |
| Lmo0069 | 0 | 0 | 0 | 0 | 0 | 0 | 0 | 0 | 0 | 2 |
| Lmo0071 | 0 | 0 | 0 | 0 | 0 | 0 | 0 | 0 | 0 | 2 |
| Lmo0072 | 0 | 0 | 0 | 2 | 0 | 2 | 2 | 0 | 0 | 2 |
| Lmo0079 | 0 | 2 | 2 | 2 | 2 | 2 | 2 | 0 | 2 | 2 |
| Lmo0080 | 0 | 0 | 2 | 2 | 2 | 2 | 2 | 0 | 0 | 2 |
| Lmo0081 | 0 | 0 | 0 | 0 | 0 | 0 | 0 | 0 | 0 | 2 |
| Lmo0082 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 |
| Lmo0106 | 2 | 2 | 2 | 2 | 2 | 2 | 2 | 2 | 2 | 2 |
| Lmo0141 | 0 | 0 | 2 | 0 | 0 | 0 | 0 | 0 | 0 | 2 |
| Lmo0143 | 0 | 0 | 0 | 2 | 0 | 2 | 2 | 0 | 0 | 2 |
| Lmo0144 | 0 | 0 | 0 | 0 | 0 | 0 | 0 | 0 | 0 | 2 |
| Lmo0147 | 2 | 2 | 2 | 2 | 2 | 2 | 2 | 2 | 2 | 2 |
| Lmo0151 | 0 | 0 | 0 | 0 | 0 | 0 | 0 | 0 | 0 | 2 |
| Lmo0171 | 2 | 2 | 2 | 2 | 2 | 2 | 2 | 2 | 2 | 2 |
| Lmo0172 | 2 | 2 | 2 | 2 | 2 | 2 | 2 | 2 | 2 | 2 |
| Lmo0252 | 0 | 0 | 0 | 2 | 2 | 2 | 2 | 0 | 0 | 2 |
| Lmo0253 | 0 | 0 | 0 | 2 | 2 | 2 | 2 | 0 | 0 | 2 |
| Lmo0304 | 2 | 2 | 2 | 2 | 2 | 2 | 2 | 2 | 0 | 2 |
| Lmo0310 | 0 | 0 | 0 | 0 | 0 | 0 | 0 | 0 | 0 | 2 |
| Lmo0320 | 0 | 2 | 2 | 2 | 2 | 2 | 2 | 2 | 2 | 2 |
| Lmo0333 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 |
| Lmo0338 | 0 | 0 | 2 | 2 | 2 | 2 | 2 | 0 | 0 | 2 |
| Lmo0378 | 2 | 2 | 2 | 2 | 2 | 2 | 2 | 2 | 2 | 2 |
| Lmo0379 | 2 | 2 | 2 | 2 | 2 | 2 | 2 | 2 | 2 | 2 |
| Lmo0380 | 2 | 2 | 2 | 2 | 2 | 2 | 2 | 0 | 2 | 2 |
| Lmo0381 | 2 | 2 | 2 | 2 | 2 | 2 | 2 | 2 | 2 | 2 |
| Lmo0409 | 2 | 2 | 2 | 0 | 0 | 0 | 0 | 2 | 2 | 2 |
| Lmo0419 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 |
| Lmo0444 | 2 | 2 | 2 | 0 | 0 | 0 | 0 | 2 | 0 | 0 |
| Lmo0448 | 2 | 2 | 2 | 0 | 0 | 0 | 0 | 2 | 0 | 2 |
| Lmo0459 | 0 | 0 | 0 | 2 | 2 | 2 | 2 | 0 | 0 | 2 |
| Lmo0462 | 0 | 0 | 0 | 2 | 2 | 2 | 2 | 0 | 0 | 2 |
| Lmo0466 | 0 | 0 | 0 | 0 | 0 | 0 | 0 | 0 | 0 | 2 |
| Lmo0467 | 0 | 0 | 0 | 0 | 0 | 0 | 0 | 0 | 0 | 2 |
| Lmo0471 | 0 | 0 | 0 | 0 | 0 | 0 | 0 | 0 | 0 | 2 |
| Lmo0472 | 0 | 0 | 2 | 2 | 2 | 2 | 2 | 2 | 2 | 2 |
| Lmo0473 | 2 | 0 | 2 | 2 | 2 | 2 | 2 | 2 | 2 | 2 |
| Lmo0474 | 2 | 2 | 0 | 2 | 2 | 2 | 2 | 2 | 2 | 2 |
| Lmo0493 | 2 | 2 | 2 | 2 | 2 | 2 | 2 | 2 | 2 | 2 |
| Lmo0497 | 2 | 2 | 2 | 2 | 2 | 2 | 2 | 2 | 2 | 2 |
| Lmo0525 | 2 | 2 | 2 | 2 | 2 | 2 | 2 | 2 | 2 | 2 |
| Lmo0549 | 2 | 2 | 2 | 2 | 2 | 2 | 2 | 2 | 2 | 2 |
| Lmo0630 | 2 | 2 | 2 | 2 | 2 | 2 | 2 | 2 | 2 | 2 |
| Lmo0631 | 2 | 2 | 2 | 2 | 2 | 2 | 2 | 2 | 2 | 2 |
| Lmo0632 | 2 | 2 | 2 | 2 | 2 | 2 | 2 | 2 | 2 | 2 |
| Lmo0732 | 2 | 2 | 2 | 2 | 2 | 2 | 2 | 2 | 2 | 2 |
| Lmo0733 | 2 | 2 | 2 | 2 | 2 | 2 | 2 | 2 | 2 | 2 |
| Lmo0734 | 2 | 2 | 2 | 2 | 2 | 2 | 2 | 2 | 2 | 2 |
| Lmo0735 | 2 | 2 | 2 | 2 | 2 | 2 | 2 | 2 | 2 | 2 |
| Lmo0739 | 2 | 2 | 2 | 2 | 2 | 2 | 2 | 2 | 2 | 2 |
| Lmo0750 | 2 | 2 | 2 | 2 | 2 | 2 | 2 | 2 | 2 | 2 |
| Lmo0751 | 2 | 2 | 2 | 2 | 2 | 2 | 2 | 0 | 2 | 2 |
| Lmo0752 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 |
| Lmo0780 | 2 | 2 | 2 | 2 | 2 | 2 | 2 | 2 | 2 | 2 |
| Lmo0801 | 0 | 2 | 0 | 2 | 2 | 2 | 2 | 2 | 2 | 0 |
| Lmo0804 | 0 | 0 | 0 | 2 | 2 | 2 | 2 | 0 | 0 | 0 |
| Lmo0832 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 |
| Lmo0842 | 2 | 2 | 2 | 2 | 2 | 2 | 2 | 2 | 2 | 2 |
| Lmo1030 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 |
| Lmo1060 | 2 | 2 | 2 | 2 | 2 | 2 | 2 | 2 | 2 | 2 |
| Lmo1061 | 2 | 2 | 2 | 2 | 2 | 2 | 2 | 2 | 2 | 2 |
| Lmo1063 | 2 | 2 | 2 | 2 | 2 | 2 | 2 | 2 | 2 | 2 |
| Lmo1076 | 2 | 2 | 2 | 2 | 2 | 2 | 2 | 2 | 2 | 2 |
| Lmo1077 | 2 | 2 | 2 | 2 | 2 | 2 | 2 | 2 | 2 | 2 |
| Lmo1080 | 2 | 2 | 2 | 2 | 2 | 2 | 2 | 2 | 2 | 2 |
| Lmo1081 | 2 | 2 | 2 | 2 | 2 | 2 | 2 | 2 | 2 | 2 |
| Lmo1082 | 2 | 2 | 2 | 2 | 2 | 2 | 2 | 2 | 2 | 2 |
| Lmo1085 | 2 | 2 | 2 | 2 | 2 | 2 | 2 | 2 | 2 | 2 |
| Lmo1088 | 2 | 2 | 2 | 2 | 2 | 2 | 2 | 2 | 2 | 2 |
| Lmo1090 | 2 | 2 | 2 | 2 | 2 | 2 | 2 | 2 | 2 | 2 |
| Lmo1091 | 2 | 2 | 2 | 2 | 2 | 2 | 2 | 2 | 2 | 2 |
| Lmo1102 | 2 | 2 | 2 | 2 | 2 | 2 | 2 | 0 | 2 | 2 |
| Lmo1115 | 2 | 2 | 2 | 2 | 2 | 2 | 2 | 2 | 2 | 2 |
| Lmo1116 | 2 | 2 | 0 | 0 | 0 | 0 | 0 | 2 | 0 | 2 |
| Lmo1118 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 |
| Lmo1120 | 2 | 2 | 2 | 2 | 2 | 2 | 2 | 2 | 2 | 2 |
| Lmo1133 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 |
| Lmo1307 | 2 | 2 | 2 | 2 | 2 | 2 | 2 | 2 | 2 | 2 |
| Lmo1441 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 |
| Lmo1597 | 2 | 2 | 0 | 2 | 2 | 2 | 2 | 2 | 0 | 2 |
| Lmo1604 | 2 | 2 | 2 | 2 | 2 | 2 | 2 | 2 | 2 | 2 |
| Lmo1648 | 2 | 0 | 2 | 2 | 2 | 0 | 2 | 2 | 2 | 2 |
| Lmo1666 | 2 | 2 | 2 | 2 | 2 | 2 | 2 | 2 | 2 | 2 |
| Lmo1714 | 0 | 0 | 2 | 2 | 2 | 2 | 2 | 0 | 0 | 2 |
| Lmo1968 | 2 | 2 | 2 | 2 | 2 | 2 | 2 | 2 | 2 | 2 |
| Lmo1973 | 2 | 2 | 2 | 2 | 2 | 2 | 2 | 2 | 2 | 2 |
| Lmo1974 | 2 | 2 | 2 | 2 | 2 | 2 | 2 | 2 | 2 | 2 |
| Lmo2026 | 2 | 2 | 2 | 0 | 0 | 0 | 0 | 2 | 0 | 2 |
| Lmo2027 | 2 | 2 | 2 | 2 | 2 | 2 | 2 | 2 | 2 | 2 |
| Lmo2276 | 2 | 2 | 2 | 2 | 2 | 2 | 2 | 2 | 2 | 2 |
| Lmo2277 | 0 | 2 | 2 | 2 | 2 | 0 | 2 | 2 | 2 | 0 |
| Lmo2311 | 0 | 2 | 2 | 2 | 2 | 2 | 2 | 0 | 0 | 2 |
| Lmo2312 | 0 | 2 | 2 | 2 | 2 | 2 | 2 | 0 | 0 | 0 |
| Lmo2324 | 2 | 2 | 0 | 2 | 2 | 2 | 2 | 0 | 2 | 0 |
| Lmo2408 | 2 | 2 | 0 | 0 | 0 | 0 | 0 | 2 | 0 | 2 |
| Lmo2443 | 2 | 2 | 2 | 2 | 2 | 2 | 2 | 2 | 0 | 2 |
| Lmo2576 | 2 | 2 | 2 | 2 | 2 | 2 | 2 | 2 | 2 | 2 |
| Lmo2594 | 2 | 2 | 2 | 0 | 0 | 0 | 0 | 2 | 2 | 2 |
| Lmo2595 | 0 | 0 | 2 | 0 | 0 | 0 | 0 | 0 | 2 | 2 |
| Lmo2671 | 2 | 2 | 2 | 2 | 2 | 2 | 2 | 2 | 2 | 2 |
| Lmo2686 | 2 | 2 | 2 | 2 | 2 | 2 | 2 | 2 | 2 | 2 |
| Lmo2732 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 |
| Lmo2786 | 2 | 2 | 2 | 2 | 2 | 2 | 2 | 2 | 2 | 2 |
| Lmo2787 | 2 | 2 | 2 | 2 | 2 | 2 | 2 | 2 | 2 | 2 |
| Lmo2807 | 0 | 0 | 0 | 2 | 2 | 2 | 2 | 0 | 2 | 2 |
| Lmo2809 | 0 | 2 | 0 | 2 | 2 | 2 | 2 | 0 | 2 | 2 |
| Lmo2821 | 2 | 2 | 2 | 2 | 2 | 2 | 2 | 2 | 2 | 2 |
| Lin0060 | 0 | 0 | 0 | 0 | 0 | 0 | 0 | 0 | 0 | 0 |
| Lin0197 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 |
| Lin0415 | 0 | 2 | 2 | 2 | 2 | 2 | 2 | 2 | 2 | 2 |
| Lin0453 | 0 | 0 | 0 | 0 | 0 | 0 | 0 | 0 | 0 | 0 |
| Lin0454 | 0 | 0 | 0 | 0 | 0 | 0 | 0 | 0 | 0 | 0 |
| Lin0455 | 0 | 0 | 0 | 0 | 0 | 0 | 0 | 0 | 0 | 0 |
| Lin0465 | 0 | 0 | 0 | 0 | 0 | 0 | 0 | 0 | 0 | 0 |
| Lin0476 | 2 | 2 | 2 | 2 | 2 | 2 | 2 | 2 | 2 | 0 |
| Lin0661 | 0 | 2 | 2 | 2 | 2 | 2 | 2 | 2 | 2 | 0 |
| Lin0665 | 0 | 2 | 2 | 0 | 2 | 2 | 2 | 2 | 2 | 2 |
| Lin0677 | 0 | 0 | 0 | 0 | 0 | 0 | 0 | 0 | 0 | 0 |
| Lin0739 | 0 | 0 | 0 | 0 | 0 | 0 | 0 | 0 | 0 | 0 |
| Lin0740 | 2 | 2 | 2 | 2 | 2 | 2 | 2 | 2 | 2 | 2 |
| Lin0803 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 |
| Lin1066 | 0 | 0 | 0 | 0 | 0 | 0 | 0 | 0 | 0 | 0 |
| Lin1069 | 0 | 0 | 0 | 0 | 0 | 0 | 0 | 0 | 0 | 0 |
| Lin1073 | 0 | 0 | 0 | 0 | 0 | 0 | 0 | 0 | 0 | 0 |
| Lin1083 | 0 | 0 | 0 | 0 | 0 | 0 | 0 | 0 | 0 | 0 |
| Lin1084 | 2 | 2 | 0 | 0 | 0 | 0 | 0 | 2 | 2 | 2 |
| Lin1090 | 0 | 0 | 0 | 0 | 0 | 0 | 0 | 0 | 0 | 0 |
| Lin1204 | 0 | 0 | 0 | 0 | 0 | 0 | 0 | 0 | 0 | 0 |
| Lin1262 | 2 | 2 | 2 | 2 | 2 | 2 | 2 | 2 | 2 | 2 |
| Lin1299 | 2 | 2 | 2 | 2 | 2 | 2 | 2 | 2 | 2 | 2 |
| Lin1328 | 2 | 2 | 2 | 2 | 2 | 2 | 2 | 2 | 2 | 2 |
| Lin1452 | 0 | 0 | 0 | 0 | 0 | 0 | 0 | 0 | 0 | 0 |
| Lin1621 | 0 | 0 | 0 | 0 | 0 | 0 | 0 | 0 | 0 | 0 |
| Lin2397 | 0 | 0 | 2 | 2 | 2 | 2 | 2 | 0 | 0 | 0 |
| Lin2494 | 2 | 2 | 2 | 2 | 2 | 2 | 2 | 2 | 2 | 2 |
| Lin2603 | 0 | 0 | 2 | 0 | 0 | 0 | 0 | 0 | 0 | 0 |
| Lin2724 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 |
| Lin2743 | 2 | 2 | 0 | 2 | 2 | 2 | 2 | 2 | 0 | 0 |
| Lin2744 | 2 | 2 | 0 | 2 | 2 | 2 | 2 | 2 | 2 | 2 |
| Lin2839 | 0 | 0 | 0 | 2 | 2 | 2 | 2 | 0 | 2 | 0 |
| Lin2918 | 0 | 0 | 0 | 0 | 0 | 0 | 0 | 0 | 0 | 0 |
| ORF 105 | 0 | 0 | 0 | 0 | 0 | 0 | 0 | 0 | 0 | 0 |
| ORF 1390 | 0 | 0 | 2 | 2 | 2 | 0 | 0 | 0 | 2 | 0 |
| ORF 1445 | 0 | 0 | 0 | 0 | 0 | 0 | 0 | 0 | 0 | 0 |
| ORF 1490 | 0 | 0 | 0 | 0 | 0 | 0 | 0 | 0 | 0 | 0 |
| ORF 1491 | 0 | 0 | 0 | 0 | 0 | 0 | 0 | 0 | 0 | 0 |
| ORF 1590 | 2 | 2 | 2 | 2 | 2 | 2 | 2 | 2 | 2 | 2 |
| ORF 1761 | 0 | 0 | 0 | 0 | 0 | 0 | 0 | 0 | 0 | 0 |
| ORF 2017 | 2 | 0 | 2 | 2 | 2 | 0 | 0 | 2 | 2 | 0 |
| ORF 2074 | 0 | 0 | 2 | 2 | 2 | 2 | 2 | 0 | 0 | 0 |
| ORF 2076 | 0 | 0 | 2 | 2 | 2 | 2 | 2 | 0 | 0 | 0 |
| ORF 2110 | 0 | 0 | 0 | 0 | 0 | 0 | 0 | 0 | 0 | 0 |
| ORF 2372 | 0 | 0 | 0 | 0 | 0 | 0 | 0 | 0 | 0 | 0 |
| ORF 2541 | 0 | 0 | 0 | 2 | 2 | 2 | 2 | 0 | 0 | 0 |
| ORF 2579 | 0 | 0 | 2 | 2 | 2 | 2 | 2 | 0 | 0 | 0 |
| ORF 2777 | 0 | 0 | 2 | 2 | 2 | 2 | 2 | 0 | 2 | 0 |
| ORF 2819 | 0 | 0 | 0 | 0 | 0 | 0 | 0 | 0 | 0 | 0 |
| ORF 2847 | 0 | 0 | 0 | 0 | 0 | 0 | 0 | 0 | 0 | 0 |
| ORF 2858 | 2 | 2 | 2 | 2 | 2 | 2 | 2 | 2 | 2 | 0 |
| ORF 3103 | 2 | 2 | 2 | 2 | 2 | 2 | 2 | 2 | 0 | 0 |
| ORF 3184 | 0 | 0 | 2 | 2 | 2 | 0 | 0 | 2 | 2 | 0 |
| ORF 3401 | 0 | 0 | 2 | 2 | 0 | 0 | 0 | 0 | 0 | 0 |
| ORF 3792 | 2 | 2 | 2 | 0 | 2 | 0 | 0 | 2 | 2 | 0 |
| ORF 3840 | 0 | 0 | 0 | 0 | 0 | 0 | 0 | 0 | 0 | 0 |
| ORF 4101 | 2 | 2 | 2 | 2 | 2 | 2 | 2 | 2 | 2 | 0 |
| ORF 660 | 2 | 2 | 0 | 0 | 0 | 0 | 0 | 2 | 0 | 0 |
| ORF 757 | 0 | 0 | 0 | 0 | 0 | 0 | 0 | 0 | 0 | 0 |
| ORF 799 | 0 | 0 | 0 | 0 | 0 | 0 | 0 | 0 | 0 | 0 |
| ORF 817 | 0 | 0 | 0 | 0 | 0 | 0 | 0 | 0 | 0 | 0 |
| Gene | CLIP61868-1/2a | CLIP87880-1/2a | CLIP87881-1/2a | CLIP87890-1/2a | CLIP87884-1/2a | CLIP87908-1/2a | CLIP74902-1/2a | CLIP14843-1/2a | CLIP60368-1/2a | CLIP69566-1/2a |
| Lmo0017 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 |
| Lmo0066 | 2 | 0 | 0 | 0 | 0 | 0 | 0 | 0 | 0 | 0 |
| Lmo0067 | 0 | 0 | 0 | 0 | 0 | 0 | 0 | 0 | 0 | 0 |
| Lmo0068 | 0 | 0 | 0 | 0 | 0 | 0 | 0 | 0 | 0 | 0 |
| Lmo0069 | 0 | 2 | 0 | 0 | 0 | 0 | 0 | 2 | 2 | 0 |
| Lmo0071 | 0 | 2 | 0 | 0 | 0 | 0 | 0 | 2 | 2 | 0 |
| Lmo0072 | 2 | 0 | 0 | 0 | 0 | 2 | 0 | 0 | 2 | 2 |
| Lmo0079 | 2 | 0 | 2 | 0 | 2 | 0 | 0 | 0 | 2 | 2 |
| Lmo0080 | 2 | 0 | 2 | 0 | 2 | 0 | 0 | 0 | 2 | 2 |
| Lmo0081 | 0 | 0 | 0 | 0 | 0 | 0 | 0 | 0 | 2 | 2 |
| Lmo0082 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 |
| Lmo0106 | 2 | 2 | 2 | 2 | 2 | 2 | 2 | 2 | 2 | 2 |
| Lmo0141 | 0 | 0 | 2 | 0 | 2 | 0 | 0 | 0 | 0 | 2 |
| Lmo0143 | 0 | 0 | 0 | 0 | 0 | 0 | 0 | 0 | 0 | 2 |
| Lmo0144 | 0 | 0 | 0 | 0 | 0 | 0 | 0 | 0 | 0 | 2 |
| Lmo0147 | 2 | 2 | 2 | 2 | 2 | 2 | 2 | 2 | 2 | 2 |
| Lmo0151 | 0 | 0 | 0 | 0 | 0 | 2 | 0 | 0 | 0 | 0 |
| Lmo0171 | 2 | 2 | 2 | 2 | 2 | 2 | 2 | 2 | 2 | 2 |
| Lmo0172 | 2 | 2 | 2 | 2 | 2 | 2 | 2 | 2 | 2 | 2 |
| Lmo0252 | 2 | 0 | 2 | 0 | 2 | 2 | 0 | 0 | 0 | 0 |
| Lmo0253 | 2 | 0 | 2 | 0 | 2 | 2 | 0 | 0 | 0 | 0 |
| Lmo0304 | 0 | 0 | 0 | 2 | 0 | 2 | 2 | 0 | 0 | 0 |
| Lmo0310 | 0 | 0 | 0 | 0 | 0 | 0 | 0 | 0 | 0 | 0 |
| Lmo0320 | 2 | 2 | 2 | 0 | 2 | 0 | 0 | 2 | 2 | 2 |
| Lmo0333 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 |
| Lmo0338 | 2 | 2 | 2 | 0 | 2 | 2 | 0 | 2 | 0 | 2 |
| Lmo0378 | 2 | 2 | 2 | 2 | 2 | 2 | 2 | 2 | 2 | 2 |
| Lmo0379 | 2 | 0 | 2 | 2 | 2 | 0 | 2 | 0 | 2 | 2 |
| Lmo0380 | 2 | 2 | 2 | 2 | 2 | 2 | 2 | 2 | 2 | 2 |
| Lmo0381 | 2 | 2 | 2 | 2 | 2 | 2 | 2 | 2 | 2 | 2 |
| Lmo0409 | 0 | 2 | 2 | 2 | 2 | 2 | 2 | 2 | 2 | 2 |
| Lmo0419 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 |
| Lmo0444 | 0 | 2 | 0 | 2 | 0 | 0 | 2 | 2 | 0 | 0 |
| Lmo0448 | 0 | 2 | 0 | 2 | 0 | 2 | 2 | 2 | 0 | 0 |
| Lmo0459 | 2 | 0 | 2 | 0 | 2 | 0 | 0 | 0 | 2 | 2 |
| Lmo0462 | 2 | 0 | 2 | 0 | 2 | 0 | 0 | 0 | 2 | 2 |
| Lmo0466 | 0 | 0 | 0 | 0 | 0 | 0 | 0 | 0 | 0 | 0 |
| Lmo0467 | 0 | 0 | 0 | 0 | 0 | 0 | 0 | 0 | 0 | 0 |
| Lmo0471 | 0 | 0 | 0 | 0 | 0 | 0 | 0 | 0 | 0 | 0 |
| Lmo0472 | 2 | 2 | 2 | 2 | 2 | 2 | 0 | 2 | 2 | 2 |
| Lmo0473 | 2 | 2 | 2 | 2 | 2 | 2 | 0 | 2 | 2 | 2 |
| Lmo0474 | 2 | 2 | 2 | 2 | 2 | 2 | 2 | 2 | 2 | 2 |
| Lmo0493 | 0 | 2 | 0 | 2 | 0 | 2 | 2 | 2 | 2 | 2 |
| Lmo0497 | 2 | 2 | 2 | 2 | 2 | 2 | 2 | 2 | 2 | 2 |
| Lmo0525 | 2 | 2 | 2 | 2 | 2 | 2 | 2 | 2 | 2 | 2 |
| Lmo0549 | 2 | 2 | 2 | 2 | 2 | 2 | 2 | 2 | 2 | 2 |
| Lmo0630 | 2 | 2 | 2 | 2 | 2 | 2 | 2 | 2 | 2 | 2 |
| Lmo0631 | 2 | 2 | 2 | 2 | 2 | 2 | 2 | 2 | 2 | 2 |
| Lmo0632 | 2 | 2 | 2 | 2 | 2 | 2 | 2 | 2 | 2 | 2 |
| Lmo0732 | 2 | 2 | 2 | 2 | 2 | 2 | 2 | 2 | 2 | 2 |
| Lmo0733 | 2 | 2 | 2 | 2 | 2 | 2 | 2 | 2 | 2 | 2 |
| Lmo0734 | 2 | 2 | 2 | 2 | 2 | 2 | 2 | 2 | 2 | 2 |
| Lmo0735 | 2 | 2 | 2 | 2 | 2 | 2 | 2 | 2 | 2 | 2 |
| Lmo0739 | 2 | 2 | 2 | 2 | 2 | 2 | 2 | 2 | 2 | 2 |
| Lmo0750 | 2 | 2 | 2 | 2 | 2 | 2 | 2 | 2 | 2 | 2 |
| Lmo0751 | 2 | 2 | 2 | 2 | 2 | 2 | 2 | 2 | 2 | 2 |
| Lmo0752 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 |
| Lmo0780 | 2 | 2 | 2 | 2 | 2 | 2 | 2 | 2 | 2 | 2 |
| Lmo0801 | 0 | 2 | 2 | 2 | 2 | 0 | 2 | 2 | 2 | 2 |
| Lmo0804 | 2 | 2 | 0 | 2 | 0 | 0 | 0 | 2 | 0 | 0 |
| Lmo0832 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 |
| Lmo0842 | 2 | 2 | 2 | 2 | 2 | 2 | 2 | 2 | 2 | 2 |
| Lmo1030 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 |
| Lmo1060 | 2 | 2 | 2 | 2 | 2 | 2 | 2 | 2 | 2 | 2 |
| Lmo1061 | 2 | 2 | 2 | 2 | 2 | 2 | 2 | 2 | 2 | 2 |
| Lmo1063 | 2 | 2 | 2 | 2 | 2 | 2 | 2 | 2 | 2 | 2 |
| Lmo1076 | 2 | 2 | 2 | 2 | 2 | 2 | 2 | 2 | 2 | 2 |
| Lmo1077 | 2 | 2 | 2 | 2 | 2 | 2 | 2 | 2 | 2 | 2 |
| Lmo1080 | 2 | 2 | 2 | 2 | 2 | 2 | 2 | 2 | 2 | 2 |
| Lmo1081 | 2 | 2 | 2 | 2 | 2 | 2 | 2 | 2 | 2 | 2 |
| Lmo1082 | 2 | 2 | 2 | 2 | 2 | 2 | 2 | 2 | 2 | 2 |
| Lmo1085 | 2 | 2 | 2 | 2 | 2 | 2 | 2 | 2 | 2 | 2 |
| Lmo1088 | 2 | 2 | 2 | 2 | 2 | 2 | 2 | 2 | 2 | 2 |
| Lmo1090 | 2 | 2 | 2 | 2 | 2 | 2 | 2 | 2 | 2 | 2 |
| Lmo1091 | 2 | 2 | 2 | 2 | 2 | 2 | 2 | 2 | 2 | 2 |
| Lmo1102 | 2 | 0 | 0 | 0 | 0 | 0 | 2 | 2 | 2 | 2 |
| Lmo1115 | 2 | 0 | 0 | 0 | 0 | 0 | 0 | 2 | 2 | 2 |
| Lmo1116 | 0 | 2 | 2 | 2 | 2 | 2 | 2 | 2 | 0 | 0 |
| Lmo1118 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 |
| Lmo1120 | 2 | 2 | 0 | 2 | 0 | 2 | 2 | 2 | 0 | 2 |
| Lmo1133 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 |
| Lmo1307 | 2 | 2 | 2 | 2 | 2 | 2 | 2 | 2 | 2 | 2 |
| Lmo1441 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 |
| Lmo1597 | 0 | 0 | 2 | 2 | 2 | 2 | 2 | 0 | 2 | 0 |
| Lmo1604 | 2 | 2 | 2 | 2 | 2 | 2 | 2 | 2 | 2 | 2 |
| Lmo1648 | 2 | 2 | 0 | 0 | 0 | 2 | 0 | 2 | 2 | 2 |
| Lmo1666 | 2 | 2 | 2 | 2 | 2 | 2 | 2 | 2 | 2 | 2 |
| Lmo1714 | 0 | 0 | 0 | 0 | 0 | 0 | 0 | 0 | 0 | 2 |
| Lmo1968 | 2 | 2 | 2 | 2 | 2 | 2 | 2 | 2 | 2 | 2 |
| Lmo1973 | 2 | 2 | 2 | 2 | 2 | 2 | 2 | 2 | 2 | 2 |
| Lmo1974 | 2 | 2 | 2 | 2 | 2 | 2 | 2 | 2 | 2 | 2 |
| Lmo2026 | 0 | 0 | 0 | 2 | 0 | 0 | 2 | 0 | 2 | 2 |
| Lmo2027 | 2 | 2 | 2 | 2 | 2 | 2 | 2 | 2 | 2 | 2 |
| Lmo2276 | 2 | 0 | 0 | 0 | 0 | 0 | 0 | 2 | 2 | 2 |
| Lmo2277 | 0 | 0 | 0 | 2 | 0 | 2 | 2 | 2 | 0 | 0 |
| Lmo2311 | 2 | 0 | 0 | 0 | 0 | 0 | 0 | 0 | 0 | 0 |
| Lmo2312 | 0 | 0 | 2 | 2 | 0 | 0 | 0 | 2 | 2 | 0 |
| Lmo2324 | 2 | 0 | 0 | 2 | 0 | 0 | 0 | 0 | 2 | 2 |
| Lmo2408 | 0 | 0 | 0 | 2 | 0 | 0 | 2 | 0 | 2 | 0 |
| Lmo2443 | 2 | 2 | 2 | 2 | 2 | 2 | 2 | 2 | 2 | 2 |
| Lmo2576 | 2 | 2 | 2 | 2 | 2 | 2 | 2 | 2 | 2 | 2 |
| Lmo2594 | 0 | 2 | 0 | 2 | 2 | 2 | 2 | 2 | 2 | 2 |
| Lmo2595 | 0 | 2 | 0 | 0 | 0 | 2 | 0 | 2 | 2 | 2 |
| Lmo2671 | 2 | 2 | 2 | 2 | 2 | 2 | 2 | 2 | 2 | 2 |
| Lmo2686 | 2 | 2 | 2 | 2 | 2 | 0 | 2 | 2 | 2 | 2 |
| Lmo2732 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 |
| Lmo2786 | 2 | 2 | 2 | 2 | 2 | 2 | 2 | 2 | 2 | 2 |
| Lmo2787 | 2 | 2 | 2 | 2 | 2 | 2 | 2 | 2 | 2 | 2 |
| Lmo2807 | 0 | 2 | 2 | 0 | 2 | 2 | 0 | 2 | 2 | 2 |
| Lmo2809 | 0 | 2 | 2 | 2 | 2 | 2 | 0 | 2 | 2 | 2 |
| Lmo2821 | 2 | 2 | 2 | 2 | 2 | 2 | 2 | 2 | 2 | 2 |
| Lin0060 | 0 | 0 | 0 | 0 | 0 | 0 | 0 | 0 | 0 | 2 |
| Lin0197 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 |
| Lin0415 | 2 | 0 | 0 | 0 | 0 | 0 | 0 | 2 | 2 | 2 |
| Lin0453 | 0 | 0 | 0 | 0 | 0 | 0 | 0 | 0 | 0 | 0 |
| Lin0454 | 0 | 0 | 0 | 0 | 0 | 0 | 0 | 0 | 0 | 0 |
| Lin0455 | 0 | 0 | 0 | 0 | 0 | 0 | 0 | 0 | 0 | 0 |
| Lin0465 | 2 | 0 | 0 | 0 | 0 | 0 | 0 | 0 | 0 | 0 |
| Lin0476 | 0 | 0 | 0 | 0 | 0 | 0 | 0 | 2 | 2 | 2 |
| Lin0661 | 0 | 0 | 0 | 0 | 0 | 0 | 0 | 2 | 2 | 2 |
| Lin0665 | 2 | 0 | 0 | 0 | 0 | 0 | 0 | 2 | 2 | 2 |
| Lin0677 | 0 | 0 | 2 | 0 | 2 | 2 | 0 | 0 | 0 | 0 |
| Lin0739 | 0 | 0 | 0 | 0 | 0 | 0 | 0 | 0 | 0 | 0 |
| Lin0740 | 2 | 0 | 0 | 0 | 0 | 0 | 0 | 2 | 2 | 2 |
| Lin0803 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 |
| Lin1066 | 0 | 0 | 0 | 0 | 0 | 0 | 0 | 0 | 0 | 0 |
| Lin1069 | 0 | 0 | 0 | 0 | 0 | 0 | 0 | 0 | 0 | 0 |
| Lin1073 | 0 | 0 | 0 | 0 | 0 | 0 | 0 | 0 | 0 | 0 |
| Lin1083 | 2 | 0 | 0 | 0 | 0 | 0 | 0 | 0 | 0 | 0 |
| Lin1084 | 2 | 2 | 0 | 2 | 0 | 2 | 2 | 2 | 0 | 0 |
| Lin1090 | 0 | 0 | 0 | 0 | 0 | 0 | 0 | 0 | 0 | 0 |
| Lin1204 | 0 | 0 | 0 | 0 | 0 | 0 | 0 | 0 | 0 | 0 |
| Lin1262 | 2 | 0 | 0 | 0 | 0 | 0 | 0 | 2 | 2 | 2 |
| Lin1299 | 2 | 0 | 0 | 0 | 0 | 2 | 0 | 2 | 2 | 2 |
| Lin1328 | 2 | 0 | 0 | 0 | 0 | 0 | 0 | 2 | 2 | 2 |
| Lin1452 | 0 | 0 | 0 | 0 | 0 | 0 | 0 | 0 | 0 | 0 |
| Lin1621 | 0 | 0 | 0 | 0 | 0 | 0 | 0 | 0 | 0 | 0 |
| Lin2397 | 2 | 0 | 0 | 0 | 0 | 2 | 0 | 0 | 0 | 0 |
| Lin2494 | 2 | 2 | 2 | 2 | 0 | 2 | 0 | 2 | 2 | 2 |
| Lin2603 | 0 | 0 | 2 | 2 | 0 | 0 | 2 | 0 | 0 | 0 |
| Lin2724 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 |
| Lin2743 | 2 | 0 | 2 | 2 | 2 | 0 | 2 | 0 | 0 | 0 |
| Lin2744 | 2 | 0 | 2 | 2 | 2 | 0 | 2 | 0 | 0 | 2 |
| Lin2839 | 0 | 0 | 0 | 0 | 0 | 0 | 0 | 0 | 2 | 0 |
| Lin2918 | 0 | 0 | 0 | 0 | 0 | 0 | 0 | 0 | 0 | 0 |
| ORF 105 | 0 | 0 | 0 | 0 | 0 | 0 | 0 | 0 | 0 | 0 |
| ORF 1390 | 0 | 0 | 0 | 0 | 0 | 0 | 0 | 0 | 0 | 0 |
| ORF 1445 | 0 | 0 | 0 | 0 | 0 | 0 | 0 | 0 | 0 | 0 |
| ORF 1490 | 0 | 0 | 0 | 0 | 0 | 0 | 0 | 0 | 0 | 0 |
| ORF 1491 | 0 | 0 | 0 | 0 | 0 | 0 | 0 | 0 | 0 | 0 |
| ORF 1590 | 2 | 2 | 2 | 2 | 2 | 2 | 2 | 2 | 2 | 2 |
| ORF 1761 | 0 | 0 | 0 | 0 | 0 | 0 | 0 | 0 | 0 | 0 |
| ORF 2017 | 0 | 0 | 0 | 0 | 0 | 0 | 0 | 0 | 0 | 0 |
| ORF 2074 | 2 | 2 | 0 | 0 | 0 | 0 | 0 | 2 | 2 | 2 |
| ORF 2076 | 2 | 2 | 0 | 0 | 0 | 0 | 0 | 2 | 2 | 2 |
| ORF 2110 | 0 | 0 | 0 | 0 | 0 | 0 | 0 | 0 | 0 | 0 |
| ORF 2372 | 0 | 0 | 0 | 0 | 0 | 0 | 0 | 0 | 0 | 0 |
| ORF 2541 | 2 | 0 | 2 | 0 | 2 | 0 | 0 | 0 | 0 | 0 |
| ORF 2579 | 2 | 2 | 0 | 0 | 0 | 0 | 0 | 2 | 2 | 2 |
| ORF 2777 | 0 | 0 | 0 | 0 | 0 | 0 | 0 | 0 | 0 | 0 |
| ORF 2819 | 0 | 0 | 0 | 0 | 0 | 0 | 0 | 0 | 0 | 0 |
| ORF 2847 | 0 | 0 | 2 | 0 | 2 | 0 | 0 | 0 | 0 | 0 |
| ORF 2858 | 0 | 0 | 0 | 0 | 0 | 0 | 2 | 2 | 2 | 2 |
| ORF 3103 | 2 | 2 | 0 | 2 | 0 | 0 | 2 | 2 | 0 | 2 |
| ORF 3184 | 0 | 0 | 0 | 0 | 0 | 0 | 0 | 2 | 0 | 0 |
| ORF 3401 | 0 | 0 | 0 | 0 | 0 | 0 | 0 | 0 | 0 | 0 |
| ORF 3792 | 0 | 2 | 2 | 2 | 2 | 2 | 2 | 2 | 2 | 0 |
| ORF 3840 | 0 | 0 | 0 | 0 | 0 | 0 | 0 | 0 | 0 | 0 |
| ORF 4101 | 0 | 0 | 0 | 2 | 0 | 0 | 2 | 2 | 0 | 0 |
| ORF 660 | 0 | 2 | 0 | 2 | 0 | 0 | 2 | 2 | 0 | 0 |
| ORF 757 | 0 | 0 | 0 | 0 | 0 | 0 | 0 | 0 | 0 | 0 |
| ORF 799 | 0 | 0 | 0 | 0 | 0 | 2 | 0 | 0 | 0 | 0 |
| ORF 817 | 0 | 0 | 0 | 0 | 0 | 0 | 0 | 0 | 0 | 0 |

| Gene | CLIP80559-1/2a | CLIP89458-1/2a | CLIP89461-1/2a | EGDe-1/2a | CLIP86436-3a | CLIP74905-3a | CLIP8053-3a | CLIP46694-1/2c |
|---|---|---|---|---|---|---|---|---|
| Lmo0017 | 2 | 2 | 2 | 2 | 2 | 2 | 2 | 2 |
| Lmo0036 | 2 | 2 | 2 | 2 | 2 | 2 | 2 | 2 |
| Lmo0037 | 2 | 2 | 2 | 2 | 2 | 2 | 2 | 2 |
| Lmo0066 | 0 | 0 | 2 | 2 | 0 | 2 | 0 | 2 |
| Lmo0067 | 0 | 0 | 0 | 2 | 0 | 0 | 0 | 2 |
| Lmo0068 | 0 | 0 | 0 | 2 | 0 | 0 | 0 | 2 |
| Lmo0069 | 0 | 0 | 0 | 2 | 0 | 0 | 0 | 2 |
| Lmo0071 | 0 | 0 | 0 | 2 | 0 | 0 | 0 | 2 |
| Lmo0072 | 0 | 0 | 0 | 2 | 2 | 2 | 0 | 2 |
| Lmo0079 | 0 | 2 | 2 | 2 | 0 | 2 | 2 | 2 |
| Lmo0080 | 0 | 0 | 0 | 2 | 0 | 0 | 2 | 2 |
| Lmo0081 | 0 | 0 | 0 | 2 | 0 | 0 | 0 | 0 |
| Lmo0082 | 2 | 2 | 2 | 2 | 2 | 2 | 2 | 2 |
| Lmo0083 | 2 | 2 | 2 | 2 | 2 | 2 | 2 | 2 |
| Lmo0094 | 2 | 2 | 2 | 2 | 2 | 2 | 2 | 2 |
| Lmo0106 | 2 | 2 | 2 | 2 | 2 | 2 | 2 | 2 |
| Lmo0141 | 0 | 2 | 0 | 2 | 0 | 0 | 2 | 2 |
| Lmo0143 | 0 | 0 | 0 | 2 | 0 | 0 | 0 | 2 |
| Lmo0144 | 0 | 0 | 0 | 2 | 0 | 0 | 0 | 2 |
| Lmo0147 | 2 | 2 | 2 | 2 | 2 | 2 | 2 | 2 |
| Lmo0151 | 0 | 0 | 0 | 2 | 0 | 0 | 0 | 2 |
| Lmo0171 | 2 | 2 | 2 | 2 | 2 | 2 | 2 | 2 |
| Lmo0172 | 2 | 2 | 2 | 2 | 2 | 2 | 2 | 2 |
| Lmo0252 | 0 | 0 | 0 | 2 | 2 | 2 | 0 | 2 |
| Lmo0253 | 0 | 0 | 0 | 2 | 2 | 2 | 0 | 2 |
| Lmo0304 | 0 | 2 | 0 | 2 | 0 | 2 | 0 | 2 |
| Lmo0310 | 0 | 0 | 0 | 2 | 0 | 0 | 0 | 2 |
| Lmo0320 | 2 | 2 | 2 | 2 | 2 | 2 | 2 | 2 |
| Lmo0333 | 2 | 2 | 2 | 2 | 2 | 2 | 2 | 2 |
| Lmo0334 | 2 | 2 | 2 | 2 | 2 | 2 | 2 | 2 |
| Lmo0338 | 0 | 2 | 0 | 2 | 2 | 2 | 2 | 2 |
| Lmo0378 | 2 | 2 | 2 | 2 | 2 | 2 | 2 | 0 |
| Lmo0379 | 2 | 2 | 2 | 2 | 2 | 0 | 2 | 2 |
| Lmo0380 | 2 | 2 | 2 | 2 | 2 | 2 | 2 | 2 |
| Lmo0381 | 2 | 2 | 2 | 2 | 2 | 2 | 2 | 2 |
| Lmo0409 | 2 | 2 | 2 | 2 | 0 | 2 | 2 | 2 |
| Lmo0419 | 2 | 2 | 2 | 2 | 2 | 2 | 2 | 2 |
| Lmo0440 | 2 | 2 | 2 | 2 | 2 | 2 | 2 | 2 |
| Lmo0444 | 0 | 2 | 0 | 2 | 0 | 0 | 0 | 2 |
| Lmo0448 | 0 | 2 | 0 | 2 | 0 | 0 | 0 | 2 |
| Lmo0459 | 0 | 0 | 0 | 2 | 2 | 0 | 2 | 2 |
| Lmo0462 | 0 | 0 | 0 | 2 | 2 | 0 | 2 | 2 |
| Lmo0466 | 0 | 0 | 0 | 2 | 0 | 0 | 2 | 2 |
| Lmo0467 | 0 | 0 | 0 | 2 | 0 | 0 | 2 | 2 |
| Lmo0471 | 0 | 0 | 0 | 2 | 0 | 0 | 2 | 2 |
| Lmo0472 | 0 | 0 | 2 | 2 | 2 | 2 | 2 | 2 |
| Lmo0473 | 2 | 2 | 2 | 2 | 2 | 2 | 2 | 2 |
| Lmo0474 | 2 | 0 | 2 | 2 | 2 | 2 | 2 | 2 |
| Lmo0493 | 2 | 2 | 2 | 2 | 0 | 2 | 2 | 2 |
| Lmo0497 | 2 | 2 | 2 | 2 | 2 | 2 | 2 | 2 |
| Lmo0525 | 2 | 2 | 2 | 2 | 2 | 2 | 2 | 2 |
| Lmo0549 | 2 | 2 | 2 | 2 | 2 | 2 | 2 | 2 |
| Lmo0630 | 2 | 2 | 2 | 2 | 2 | 2 | 2 | 2 |
| Lmo0631 | 2 | 2 | 2 | 2 | 2 | 2 | 2 | 2 |
| Lmo0632 | 2 | 2 | 2 | 2 | 2 | 2 | 2 | 2 |
| Lmo0732 | 2 | 2 | 2 | 2 | 2 | 2 | 2 | 2 |
| Lmo0733 | 2 | 2 | 2 | 2 | 2 | 2 | 2 | 2 |
| Lmo0734 | 2 | 2 | 2 | 2 | 2 | 2 | 2 | 2 |
| Lmo0735 | 2 | 2 | 2 | 2 | 2 | 2 | 2 | 2 |
| Lmo0739 | 2 | 2 | 2 | 2 | 2 | 2 | 2 | 2 |
| Lmo0750 | 2 | 2 | 2 | 2 | 2 | 2 | 2 | 2 |
| Lmo0751 | 2 | 2 | 2 | 2 | 2 | 2 | 2 | 2 |
| Lmo0752 | 2 | 2 | 2 | 2 | 2 | 2 | 2 | 2 |
| Lmo0753 | 2 | 2 | 2 | 2 | 2 | 2 | 2 | 2 |
| Lmo0780 | 2 | 2 | 2 | 2 | 2 | 2 | 2 | 2 |
| Lmo0801 | 2 | 0 | 2 | 2 | 0 | 0 | 2 | 2 |
| Lmo0804 | 0 | 0 | 0 | 2 | 2 | 2 | 2 | 2 |
| Lmo0832 | 2 | 2 | 2 | 2 | 2 | 2 | 2 | 2 |
| Lmo0833 | 2 | 2 | 2 | 2 | 2 | 2 | 2 | 2 |
| Lmo0834 | 2 | 2 | 2 | 2 | 2 | 2 | 2 | 2 |
| Lmo0835 | 2 | 2 | 2 | 2 | 2 | 2 | 2 | 2 |
| Lmo0842 | 2 | 2 | 2 | 2 | 2 | 2 | 2 | 2 |
| Lmo1030 | 2 | 2 | 2 | 2 | 2 | 2 | 2 | 2 |
| Lmo1036 | 2 | 2 | 2 | 2 | 2 | 2 | 2 | 2 |
| Lmo1060 | 2 | 2 | 2 | 2 | 2 | 2 | 2 | 2 |
| Lmo1061 | 2 | 2 | 2 | 2 | 2 | 2 | 2 | 2 |
| Lmo1063 | 2 | 2 | 2 | 2 | 2 | 2 | 2 | 2 |
| Lmo1076 | 2 | 2 | 2 | 2 | 2 | 2 | 2 | 2 |
| Lmo1077 | 2 | 2 | 2 | 2 | 2 | 2 | 2 | 2 |
| Lmo1080 | 2 | 2 | 2 | 2 | 2 | 2 | 2 | 2 |
| Lmo1081 | 2 | 2 | 2 | 2 | 2 | 2 | 2 | 2 |
| Lmo1082 | 2 | 2 | 2 | 2 | 2 | 2 | 2 | 2 |
| Lmo1085 | 2 | 2 | 2 | 2 | 2 | 2 | 2 | 2 |
| Lmo1088 | 2 | 2 | 2 | 2 | 2 | 2 | 2 | 2 |
| Lmo1090 | 2 | 2 | 2 | 2 | 2 | 2 | 2 | 2 |
| Lmo1091 | 2 | 2 | 2 | 2 | 2 | 2 | 2 | 2 |
| Lmo1102 | 2 | 2 | 2 | 2 | 2 | 2 | 2 | 2 |
| Lmo1115 | 2 | 2 | 2 | 2 | 2 | 2 | 2 | 2 |
| Lmo1116 | 0 | 0 | 0 | 2 | 0 | 2 | 0 | 2 |
| Lmo1118 | 0 | 0 | 0 | 2 | 0 | 0 | 0 | 2 |
| Lmo1119 | 0 | 0 | 0 | 2 | 0 | 0 | 0 | 2 |
| Lmo1120 | 2 | 2 | 2 | 2 | 2 | 0 | 2 | 2 |
| Lmo1133 | 2 | 2 | 2 | 2 | 2 | 2 | 2 | 2 |
| Lmo1135 | 2 | 2 | 2 | 2 | 2 | 2 | 2 | 2 |
| Lmo1307 | 2 | 2 | 2 | 2 | 2 | 2 | 2 | 2 |
| Lmo1441 | 2 | 2 | 2 | 2 | 2 | 2 | 2 | 2 |
| Lmo1451 | 2 | 2 | 2 | 2 | 2 | 2 | 2 | 2 |
| Lmo1597 | 0 | 0 | 0 | 2 | 0 | 2 | 2 | 2 |
| Lmo1604 | 2 | 2 | 2 | 2 | 2 | 2 | 2 | 2 |
| Lmo1648 | 2 | 2 | 2 | 2 | 2 | 2 | 2 | 2 |
| Lmo1666 | 2 | 2 | 2 | 2 | 2 | 2 | 2 | 2 |
| Lmo1714 | 0 | 2 | 0 | 2 | 0 | 2 | 2 | 2 |
| Lmo1968 | 2 | 2 | 2 | 2 | 2 | 2 | 2 | 2 |
| Lmo1973 | 2 | 2 | 2 | 2 | 2 | 2 | 2 | 2 |
| Lmo1974 | 2 | 2 | 2 | 2 | 2 | 2 | 2 | 2 |
| Lmo2026 | 0 | 2 | 0 | 2 | 0 | 0 | 0 | 2 |
| Lmo2027 | 2 | 2 | 2 | 2 | 0 | 2 | 2 | 2 |
| Lmo2276 | 2 | 2 | 2 | 2 | 2 | 2 | 2 | 2 |
| Lmo2277 | 2 | 0 | 2 | 2 | 2 | 0 | 0 | 2 |
| Lmo2311 | 0 | 2 | 2 | 2 | 0 | 0 | 0 | 2 |
| Lmo2312 | 2 | 2 | 2 | 2 | 0 | 0 | 2 | 2 |
| Lmo2324 | 2 | 2 | 2 | 2 | 2 | 2 | 0 | 2 |
| Lmo2408 | 0 | 0 | 2 | 2 | 0 | 0 | 2 | 2 |
| Lmo2443 | 0 | 2 | 0 | 2 | 2 | 2 | 2 | 2 |
| Lmo2576 | 2 | 2 | 2 | 2 | 2 | 2 | 2 | 2 |
| Lmo2594 | 2 | 2 | 2 | 2 | 0 | 2 | 2 | 2 |
| Lmo2595 | 2 | 2 | 2 | 2 | 0 | 2 | 2 | 2 |
| Lmo2671 | 2 | 2 | 2 | 2 | 2 | 2 | 2 | 2 |
| Lmo2686 | 2 | 2 | 2 | 2 | 2 | 0 | 2 | 2 |
| Lmo2732 | 2 | 2 | 2 | 2 | 2 | 2 | 2 | 2 |
| Lmo2736 | 2 | 2 | 2 | 2 | 2 | 2 | 2 | 2 |
| Lmo2786 | 2 | 2 | 2 | 2 | 2 | 2 | 2 | 2 |
| Lmo2787 | 2 | 2 | 2 | 2 | 2 | 2 | 2 | 2 |
| Lmo2807 | 2 | 0 | 2 | 2 | 0 | 0 | 2 | 2 |
| Lmo2809 | 2 | 0 | 2 | 2 | 0 | 2 | 0 | 2 |
| Lmo2821 | 2 | 2 | 2 | 2 | 2 | 2 | 2 | 2 |
| Lin0060 | 0 | 0 | 0 | 0 | 0 | 0 | 0 | 0 |
| Lin0197 | 0 | 0 | 0 | 0 | 0 | 0 | 0 | 0 |
| Lin0332 | 0 | 0 | 0 | 0 | 0 | 0 | 0 | 0 |
| Lin0397 | 0 | 0 | 0 | 0 | 0 | 0 | 0 | 0 |
| Lin0415 | 2 | 2 | 2 | 0 | 2 | 2 | 2 | 2 |
| Lin0453 | 0 | 0 | 0 | 0 | 0 | 0 | 0 | 0 |
| Lin0454 | 0 | 0 | 0 | 0 | 0 | 0 | 0 | 0 |
| Lin0455 | 0 | 0 | 0 | 0 | 0 | 0 | 0 | 0 |
| Lin0465 | 0 | 0 | 0 | 0 | 2 | 2 | 0 | 0 |
| Lin0476 | 0 | 2 | 2 | 0 | 2 | 0 | 2 | 0 |
| Lin0661 | 0 | 2 | 2 | 0 | 2 | 0 | 0 | 0 |
| Lin0665 | 2 | 2 | 2 | 0 | 0 | 2 | 0 | 2 |
| Lin0677 | 0 | 0 | 0 | 0 | 0 | 0 | 0 | 0 |
| Lin0739 | 0 | 0 | 0 | 0 | 0 | 0 | 0 | 0 |
| Lin0740 | 2 | 2 | 2 | 0 | 2 | 2 | 2 | 2 |
| Lin0803 | 0 | 0 | 0 | 0 | 0 | 0 | 0 | 0 |
| Lin0868 | 0 | 0 | 0 | 0 | 0 | 0 | 0 | 0 |
| Lin1066 | 0 | 0 | 0 | 0 | 0 | 0 | 0 | 0 |
| Lin1069 | 0 | 0 | 0 | 0 | 0 | 0 | 0 | 0 |
| Lin1073 | 0 | 0 | 0 | 0 | 0 | 0 | 0 | 0 |
| Lin1083 | 0 | 0 | 0 | 0 | 2 | 0 | 0 | 0 |
| Lin1084 | 2 | 0 | 2 | 2 | 2 | 0 | 2 | 0 |
| Lin1090 | 0 | 0 | 0 | 0 | 0 | 0 | 0 | 0 |
| Lin1204 | 0 | 0 | 0 | 0 | 0 | 0 | 0 | 0 |
| Lin1262 | 2 | 2 | 2 | 0 | 2 | 2 | 2 | 2 |
| Lin1299 | 2 | 2 | 2 | 0 | 2 | 2 | 2 | 2 |
| Lin1328 | 2 | 2 | 2 | 0 | 2 | 2 | 2 | 2 |
| Lin1452 | 0 | 0 | 0 | 0 | 0 | 0 | 0 | 0 |
| Lin1621 | 0 | 0 | 0 | 0 | 0 | 0 | 0 | 0 |
| Lin2397 | 0 | 2 | 0 | 0 | 2 | 2 | 2 | 0 |
| Lin2494 | 2 | 0 | 2 | 2 | 2 | 2 | 2 | 2 |
| Lin2603 | 0 | 2 | 2 | 2 | 0 | 0 | 2 | 2 |
| Lin2724 | 0 | 0 | 0 | 0 | 0 | 0 | 0 | 0 |
| Lin2741 | 0 | 0 | 0 | 0 | 0 | 0 | 0 | 0 |
| Lin2743 | 0 | 0 | 0 | 2 | 2 | 0 | 0 | 0 |
| Lin2744 | 0 | 0 | 2 | 2 | 2 | 0 | 0 | 2 |
| Lin2839 | 2 | 0 | 2 | 0 | 0 | 0 | 0 | 0 |
| Lin2918 | 0 | 0 | 0 | 0 | 0 | 0 | 0 | 0 |
| ORF 105 | 0 | 0 | 0 | 0 | 0 | 0 | 0 | 0 |
| ORF 1390 | 0 | 0 | 2 | 2 | 0 | 2 | 0 | 0 |
| ORF 1445 | 0 | 0 | 0 | 0 | 0 | 0 | 0 | 0 |
| ORF 1490 | 0 | 0 | 0 | 0 | 0 | 0 | 0 | 0 |
| ORF 1491 | 0 | 0 | 0 | 0 | 0 | 0 | 0 | 0 |
| ORF 1590 | 2 | 2 | 2 | 2 | 2 | 2 | 2 | 0 |
| ORF 1761 | 0 | 0 | 0 | 0 | 0 | 0 | 0 | 0 |
| ORF 2017 | 0 | 0 | 2 | 0 | 0 | 0 | 0 | 0 |
| ORF 2074 | 0 | 2 | 0 | 0 | 2 | 0 | 2 | 0 |
| ORF 2076 | 0 | 2 | 0 | 0 | 2 | 0 | 2 | 0 |
| ORF 2110 | 0 | 0 | 0 | 0 | 0 | 0 | 0 | 0 |
| ORF 2372 | 0 | 0 | 0 | 0 | 0 | 0 | 0 | 0 |
| ORF 2541 | 0 | 0 | 0 | 0 | 2 | 0 | 0 | 0 |
| ORF 2579 | 0 | 2 | 2 | 0 | 2 | 0 | 2 | 0 |
| ORF 2777 | 2 | 2 | 2 | 0 | 0 | 0 | 0 | 0 |
| ORF 2819 | 0 | 0 | 0 | 0 | 0 | 0 | 0 | 0 |
| ORF 2847 | 0 | 0 | 0 | 0 | 0 | 0 | 0 | 0 |
| ORF 2858 | 2 | 2 | 2 | 2 | 2 | 2 | 2 | 2 |
| ORF 3103 | 0 | 2 | 0 | 2 | 2 | 2 | 0 | 0 |
| ORF 3184 | 0 | 0 | 2 | 0 | 0 | 0 | 0 | 0 |
| ORF 3401 | 0 | 0 | 2 | 0 | 0 | 0 | 0 | 0 |
| ORF 3792 | 2 | 2 | 2 | 2 | 0 | 0 | 0 | 2 |
| ORF 3840 | 0 | 0 | 0 | 0 | 0 | 0 | 0 | 0 |
| ORF 4101 | 2 | 2 | 2 | 2 | 0 | 0 | 0 | 2 |
| ORF 660 | 0 | 0 | 0 | 2 | 0 | 2 | 0 | 0 |
| ORF 757 | 0 | 0 | 2 | 0 | 0 | 0 | 0 | 0 |
| ORF 799 | 0 | 0 | 0 | 0 | 0 | 2 | 0 | 0 |
| ORF 817 | 0 | 0 | 0 | 0 | 0 | 0 | 0 | 0 |

| | | | CLIP42653- | | | CLIP87892- | | | | | |
|---|---|---|---|---|---|---|---|---|---|---|---|
| Gene | CLIP85412-3c | CLIP42972-1/2c | 1/2c | CLIP2710-1/2c | CLIP87891-1/2c | 1/2c | CLIP87893-1/2c | CLIP11550-1/2c | LO028-1/2c | CLIP11962-3c | CLIP74907 3c |
| Lmo0017 | 2 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 | 2 |
| Lmo0066 | 2 | 2 | 2 | 2 | 2 | 2 | 2 | 2 | 2 | 2 | 2 |
| Lmo0067 | 2 | 2 | 2 | 2 | 2 | 2 | 2 | 2 | 2 | 2 | 2 |
| Lmo0068 | 2 | 2 | 2 | 2 | 2 | 2 | 2 | 2 | 2 | 2 | 2 |
| Lmo0069 | 2 | 2 | 2 | 2 | 2 | 2 | 2 | 2 | 2 | 2 | 2 |
| Lmo0071 | 2 | 2 | 2 | 2 | 2 | 2 | 2 | 2 | 2 | 2 | 2 |
| Lmo0072 | 2 | 2 | 2 | 2 | 2 | 2 | 2 | 2 | 2 | 2 | 2 |
| Lmo0079 | 2 | 0 | 2 | 2 | 2 | 2 | 2 | 2 | 2 | 2 | 2 |
| Lmo0080 | 2 | 2 | 2 | 2 | 2 | 2 | 2 | 2 | 2 | 2 | 2 |
| Lmo0081 | 2 | 2 | 2 | 2 | 2 | 2 | 2 | 2 | 2 | 2 | 2 |
| Lmo0082 | 2 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 | 2 |
| Lmo0106 | 2 | 2 | 2 | 2 | 2 | 2 | 2 | 2 | 2 | 2 | 2 |
| Lmo0141 | 2 | 2 | 2 | 2 | 2 | 2 | 2 | 2 | 2 | 2 | 2 |
| Lmo0143 | 2 | 2 | 2 | 2 | 2 | 2 | 2 | 2 | 2 | 2 | 2 |
| Lmo0144 | 2 | 2 | 2 | 2 | 2 | 2 | 2 | 2 | 2 | 2 | 2 |
| Lmo0147 | 2 | 2 | 2 | 2 | 2 | 2 | 2 | 2 | 2 | 2 | 2 |
| Lmo0151 | 2 | 2 | 2 | 2 | 2 | 2 | 2 | 2 | 2 | 2 | 2 |
| Lmo0171 | 2 | 2 | 2 | 2 | 2 | 2 | 2 | 2 | 2 | 2 | 2 |
| Lmo0172 | 2 | 2 | 2 | 2 | 2 | 2 | 2 | 2 | 2 | 2 | 2 |
| Lmo0252 | 2 | 2 | 2 | 2 | 2 | 2 | 2 | 2 | 2 | 2 | 2 |
| Lmo0253 | 2 | 2 | 2 | 2 | 2 | 2 | 2 | 2 | 2 | 2 | 2 |
| Lmo0304 | 2 | 2 | 2 | 2 | 2 | 2 | 2 | 2 | 2 | 2 | 2 |
| Lmo0310 | 2 | 2 | 2 | 2 | 2 | 2 | 2 | 2 | 2 | 2 | 2 |
| Lmo0320 | 2 | 2 | 2 | 2 | 2 | 2 | 2 | 2 | 2 | 2 | 2 |
| Lmo0333 | 2 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 | 2 |
| Lmo0338 | 2 | 2 | 2 | 2 | 2 | 2 | 2 | 2 | 2 | 2 | 2 |
| Lmo0378 | 2 | 2 | 2 | 2 | 2 | 2 | 2 | 2 | 2 | 2 | 2 |
| Lmo0379 | 2 | 2 | 2 | 2 | 2 | 2 | 2 | 2 | 2 | 2 | 2 |
| Lmo0380 | 2 | 0 | 2 | 2 | 2 | 2 | 2 | 2 | 2 | 2 | 2 |
| Lmo0381 | 2 | 2 | 2 | 2 | 2 | 2 | 2 | 2 | 2 | 2 | 2 |
| Lmo0409 | 2 | 2 | 2 | 2 | 2 | 2 | 2 | 2 | 2 | 2 | 2 |
| Lmo0419 | 2 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 | 2 |
| Lmo0444 | 2 | 2 | 2 | 2 | 2 | 2 | 2 | 2 | 2 | 2 | 2 |
| Lmo0448 | 2 | 2 | 2 | 2 | 2 | 2 | 2 | 2 | 2 | 2 | 2 |
| Lmo0459 | 2 | 2 | 2 | 2 | 2 | 2 | 2 | 2 | 2 | 2 | 2 |
| Lmo0462 | 2 | 0 | 2 | 2 | 2 | 2 | 2 | 2 | 2 | 2 | 2 |
| Lmo0466 | 2 | 2 | 2 | 2 | 2 | 2 | 2 | 2 | 2 | 2 | 2 |
| Lmo0467 | 2 | 2 | 2 | 2 | 2 | 2 | 2 | 2 | 2 | 2 | 2 |
| Lmo0471 | 2 | 2 | 2 | 2 | 2 | 2 | 2 | 2 | 2 | 2 | 2 |
| Lmo0472 | 2 | 2 | 2 | 2 | 2 | 2 | 2 | 2 | 2 | 2 | 2 |
| Lmo0473 | 2 | 2 | 2 | 2 | 2 | 2 | 2 | 2 | 2 | 2 | 2 |
| Lmo0474 | 2 | 2 | 2 | 2 | 2 | 2 | 2 | 2 | 2 | 2 | 2 |
| Lmo0493 | 2 | 2 | 2 | 2 | 2 | 2 | 2 | 2 | 2 | 2 | 2 |
| Lmo0497 | 2 | 2 | 2 | 2 | 2 | 2 | 2 | 2 | 2 | 2 | 2 |
| Lmo0525 | 2 | 2 | 2 | 2 | 2 | 2 | 2 | 2 | 2 | 2 | 2 |
| Lmo0549 | 2 | 2 | 2 | 2 | 2 | 2 | 2 | 2 | 2 | 2 | 2 |
| Lmo0630 | 2 | 2 | 2 | 2 | 2 | 2 | 2 | 2 | 2 | 2 | 2 |
| Lmo0631 | 2 | 2 | 2 | 2 | 2 | 2 | 2 | 2 | 2 | 2 | 2 |
| Lmo0632 | 2 | 2 | 2 | 2 | 2 | 2 | 2 | 2 | 2 | 2 | 2 |
| Lmo0732 | 2 | 2 | 2 | 2 | 2 | 2 | 2 | 2 | 2 | 2 | 2 |
| Lmo0733 | 2 | 2 | 2 | 2 | 2 | 2 | 2 | 2 | 2 | 2 | 2 |
| Lmo0734 | 2 | 2 | 2 | 2 | 2 | 2 | 2 | 2 | 2 | 2 | 2 |
| Lmo0735 | 2 | 2 | 2 | 2 | 2 | 2 | 2 | 2 | 2 | 2 | 2 |
| Lmo0739 | 2 | 2 | 2 | 2 | 2 | 2 | 2 | 2 | 2 | 2 | 2 |
| Lmo0750 | 2 | 2 | 2 | 2 | 2 | 2 | 2 | 2 | 2 | 2 | 2 |
| Lmo0751 | 2 | 2 | 2 | 2 | 2 | 2 | 2 | 2 | 2 | 2 | 2 |
| Lmo0752 | 2 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 | 2 |
| Lmo0780 | 2 | 2 | 2 | 2 | 2 | 2 | 2 | 2 | 2 | 2 | 2 |
| Lmo0801 | 2 | 2 | 2 | 2 | 2 | 2 | 2 | 2 | 2 | 2 | 2 |
| Lmo0804 | 2 | 2 | 2 | 2 | 2 | 2 | 2 | 2 | 2 | 2 | 2 |
| Lmo0832 | 2 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 | 2 |
| Lmo0842 | 2 | 2 | 2 | 2 | 2 | 2 | 2 | 2 | 2 | 2 | 2 |
| Lmo1030 | 2 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 | 2 |
| Lmo1060 | 2 | 2 | 2 | 2 | 2 | 2 | 2 | 2 | 2 | 2 | 2 |
| Lmo1061 | 2 | 2 | 2 | 2 | 2 | 2 | 2 | 2 | 2 | 2 | 2 |
| Lmo1063 | 2 | 2 | 2 | 2 | 2 | 2 | 2 | 2 | 2 | 2 | 2 |
| Lmo1076 | 2 | 2 | 2 | 2 | 2 | 2 | 2 | 2 | 2 | 2 | 2 |
| Lmo1077 | 2 | 2 | 2 | 2 | 2 | 2 | 2 | 2 | 2 | 2 | 2 |
| Lmo1080 | 2 | 2 | 2 | 2 | 2 | 2 | 2 | 2 | 2 | 2 | 2 |
| Lmo1081 | 2 | 2 | 2 | 2 | 2 | 2 | 2 | 2 | 2 | 2 | 2 |
| Lmo1082 | 2 | 2 | 2 | 2 | 2 | 2 | 2 | 2 | 2 | 2 | 2 |
| Lmo1085 | 2 | 2 | 2 | 2 | 2 | 2 | 2 | 2 | 2 | 2 | 2 |
| Lmo1088 | 2 | 2 | 2 | 2 | 2 | 2 | 2 | 2 | 2 | 2 | 2 |
| Lmo1090 | 2 | 2 | 2 | 2 | 2 | 2 | 2 | 2 | 2 | 2 | 2 |
| Lmo1091 | 2 | 2 | 2 | 2 | 2 | 2 | 2 | 2 | 2 | 2 | 2 |
| Lmo1102 | 2 | 2 | 2 | 2 | 2 | 2 | 2 | 2 | 2 | 2 | 2 |
| Lmo1115 | 2 | 2 | 2 | 2 | 0 | 0 | 0 | 2 | 2 | 2 | 2 |
| Lmo1116 | 2 | 2 | 2 | 2 | 2 | 2 | 2 | 2 | 2 | 2 | 2 |
| Lmo1118 | 2 | 2 | 2 | 2 | 2 | 2 | 2 | 2 | 2 | 2 | 2 |
| Lmo1119 | 2 | 2 | 2 | 2 | 2 | 2 | 2 | 2 | 2 | 2 | 2 |
| Lmo1120 | 2 | 2 | 2 | 2 | 2 | 2 | 2 | 2 | 2 | 2 | 2 |
| Lmo1133 | 2 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 | 2 |
| Lmo1307 | 2 | 2 | 2 | 2 | 2 | 2 | 2 | 2 | 2 | 2 | 2 |
| Lmo1441 | 2 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 | 2 |
| Lmo1597 | 2 | 2 | 2 | 2 | 2 | 2 | 2 | 2 | 2 | 2 | 2 |
| Lmo1604 | 2 | 2 | 2 | 2 | 2 | 2 | 2 | 2 | 2 | 2 | 2 |
| Lmo1648 | 2 | 2 | 2 | 2 | 2 | 2 | 2 | 2 | 2 | 2 | 2 |
| Lmo1666 | 2 | 2 | 2 | 2 | 2 | 2 | 2 | 2 | 2 | 2 | 2 |
| Lmo1714 | 2 | 2 | 2 | 2 | 2 | 2 | 2 | 2 | 2 | 2 | 2 |
| Lmo1968 | 2 | 2 | 2 | 2 | 2 | 2 | 2 | 2 | 2 | 2 | 2 |
| Lmo1973 | 2 | 2 | 2 | 2 | 2 | 2 | 2 | 2 | 2 | 2 | 2 |
| Lmo1974 | 2 | 2 | 2 | 2 | 2 | 2 | 2 | 2 | 2 | 2 | 2 |
| Lmo2026 | 2 | 2 | 2 | 2 | 2 | 2 | 2 | 2 | 2 | 2 | 2 |
| Lmo2027 | 2 | 2 | 2 | 2 | 2 | 2 | 2 | 2 | 2 | 2 | 2 |
| Lmo2276 | 2 | 2 | 2 | 2 | 0 | 0 | 0 | 2 | 2 | 2 | 2 |
| Lmo2277 | 0 | 0 | 0 | 0 | 0 | 2 | 2 | 0 | 2 | 0 | 2 |
| Lmo2311 | 2 | 0 | 2 | 2 | 0 | 2 | 2 | 2 | 2 | 2 | 0 |
| Lmo2312 | 2 | 2 | 2 | 2 | 2 | 0 | 0 | 0 | 2 | 2 | 2 |
| Lmo2324 | 0 | 0 | 2 | 2 | 2 | 2 | 2 | 2 | 2 | 2 | 2 |
| Lmo2408 | 2 | 2 | 2 | 2 | 2 | 2 | 2 | 2 | 2 | 2 | 2 |
| Lmo2443 | 2 | 2 | 2 | 2 | 2 | 2 | 2 | 2 | 2 | 2 | 2 |
| Lmo2576 | 2 | 2 | 2 | 2 | 2 | 2 | 2 | 2 | 2 | 2 | 2 |
| Lmo2594 | 2 | 0 | 2 | 2 | 2 | 2 | 2 | 2 | 2 | 2 | 2 |
| Lmo2595 | 2 | 2 | 2 | 2 | 2 | 2 | 2 | 2 | 2 | 2 | 2 |
| Lmo2671 | 2 | 2 | 2 | 2 | 2 | 2 | 2 | 2 | 2 | 2 | 2 |
| Lmo2686 | 2 | 2 | 2 | 2 | 2 | 2 | 2 | 2 | 2 | 2 | 2 |
| Lmo2732 | 2 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 | 2 |
| Lmo2786 | 2 | 2 | 2 | 2 | 2 | 2 | 2 | 2 | 2 | 2 | 2 |
| Lmo2787 | 2 | 2 | 2 | 2 | 2 | 2 | 2 | 2 | 2 | 2 | 2 |
| Lmo2807 | 2 | 2 | 2 | 2 | 2 | 2 | 2 | 2 | 2 | 2 | 2 |
| Lmo2809 | 2 | 0 | 2 | 2 | 2 | 2 | 2 | 2 | 2 | 2 | 2 |
| Lmo2821 | 2 | 2 | 2 | 2 | 2 | 2 | 2 | 2 | 2 | 2 | 2 |
| Lin0060 | 0 | 0 | 0 | 0 | 0 | 0 | 0 | 0 | 0 | 0 | 0 |
| Lin0197 | 0 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 | 0 |
| Lin0415 | 2 | 2 | 2 | 2 | 0 | 0 | 0 | 2 | 2 | 2 | 2 |
| Lin0453 | 0 | 0 | 0 | 0 | 0 | 0 | 0 | 0 | 0 | 0 | 0 |
| Lin0454 | 0 | 0 | 0 | 0 | 0 | 0 | 0 | 0 | 0 | 0 | 0 |
| Lin0455 | 0 | 0 | 0 | 0 | 0 | 0 | 0 | 0 | 0 | 0 | 0 |
| Lin0465 | 0 | 0 | 0 | 0 | 0 | 0 | 0 | 0 | 0 | 0 | 0 |
| Lin0476 | 2 | 2 | 2 | 2 | 0 | 0 | 0 | 2 | 2 | 2 | 2 |
| Lin0661 | 2 | 2 | 2 | 2 | 0 | 0 | 0 | 0 | 2 | 0 | 2 |
| Lin0665 | 2 | 2 | 0 | 2 | 0 | 0 | 0 | 0 | 2 | 2 | 2 |
| Lin0677 | 0 | 0 | 0 | 0 | 0 | 0 | 0 | 0 | 0 | 0 | 0 |
| Lin0739 | 0 | 0 | 0 | 0 | 0 | 0 | 0 | 0 | 0 | 0 | 0 |
| Lin0740 | 2 | 2 | 2 | 2 | 0 | 0 | 0 | 2 | 2 | 2 | 2 |
| Lin0803 | 0 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 | 0 |
| Lin1066 | 0 | 0 | 0 | 0 | 0 | 0 | 0 | 0 | 0 | 0 | 0 |
| Lin1069 | 0 | 0 | 0 | 0 | 0 | 0 | 0 | 0 | 0 | 0 | 0 |
| Lin1073 | 0 | 0 | 0 | 0 | 0 | 0 | 0 | 0 | 0 | 0 | 0 |
| Lin1083 | 0 | 0 | 0 | 0 | 0 | 0 | 0 | 0 | 0 | 0 | 0 |
| Lin1084 | 0 | 0 | 0 | 0 | 0 | 0 | 0 | 0 | 0 | 0 | 0 |
| Lin1090 | 0 | 0 | 0 | 0 | 0 | 0 | 0 | 0 | 0 | 0 | 0 |
| Lin1204 | 0 | 0 | 0 | 0 | 0 | 0 | 0 | 0 | 0 | 0 | 0 |
| Lin1262 | 2 | 2 | 2 | 2 | 0 | 0 | 0 | 2 | 2 | 2 | 2 |
| Lin1299 | 2 | 2 | 2 | 2 | 0 | 2 | 2 | 2 | 2 | 2 | 2 |
| Lin1328 | 2 | 2 | 2 | 2 | 0 | 0 | 0 | 2 | 2 | 2 | 2 |
| Lin1452 | 0 | 0 | 0 | 0 | 0 | 0 | 0 | 0 | 0 | 0 | 0 |
| Lin1621 | 0 | 0 | 0 | 0 | 0 | 0 | 0 | 0 | 0 | 0 | 0 |
| Lin2397 | 0 | 0 | 0 | 0 | 2 | 2 | 2 | 0 | 2 | 0 | 0 |
| Lin2494 | 2 | 2 | 2 | 2 | 2 | 2 | 2 | 2 | 2 | 2 | 2 |
| Lin2603 | 0 | 0 | 2 | 2 | 2 | 0 | 0 | 0 | 2 | 2 | 0 |
| Lin2724 | 0 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 | 0 |
| Lin2743 | 0 | 0 | 0 | 0 | 0 | 0 | 0 | 0 | 0 | 0 | 0 |
| Lin2744 | 2 | 2 | 2 | 2 | 0 | 0 | 0 | 0 | 2 | 2 | 2 |
| Lin2839 | 0 | 0 | 0 | 0 | 0 | 0 | 0 | 0 | 0 | 0 | 0 |
| Lin2918 | 0 | 0 | 0 | 0 | 0 | 0 | 0 | 0 | 0 | 0 | 0 |
| ORF 105 | 0 | 0 | 0 | 0 | 0 | 0 | 0 | 0 | 0 | 0 | 0 |
| ORF 1390 | 0 | 0 | 0 | 0 | 0 | 2 | 0 | 0 | 0 | 0 | 0 |
| ORF 1445 | 0 | 0 | 0 | 0 | 0 | 0 | 0 | 0 | 0 | 0 | 0 |
| ORF 1490 | 0 | 0 | 0 | 0 | 0 | 0 | 0 | 0 | 0 | 0 | 0 |
| ORF 1491 | 0 | 0 | 0 | 0 | 0 | 0 | 0 | 0 | 0 | 0 | 0 |
| ORF 1590 | 0 | 0 | 0 | 0 | 0 | 0 | 0 | 0 | 0 | 0 | 0 |
| ORF 1761 | 0 | 0 | 0 | 0 | 0 | 0 | 0 | 0 | 0 | 0 | 0 |
| ORF 2017 | 0 | 0 | 0 | 0 | 0 | 0 | 0 | 0 | 0 | 0 | 0 |
| ORF 2074 | 0 | 0 | 0 | 0 | 0 | 0 | 0 | 0 | 0 | 0 | 0 |
| ORF 2076 | 0 | 0 | 0 | 0 | 0 | 0 | 0 | 0 | 0 | 0 | 0 |
| ORF 2110 | 0 | 0 | 0 | 0 | 0 | 0 | 0 | 0 | 0 | 0 | 0 |
| ORF 2372 | 0 | 0 | 0 | 0 | 0 | 0 | 0 | 0 | 0 | 0 | 0 |
| ORF 2541 | 0 | 0 | 0 | 0 | 0 | 0 | 0 | 0 | 0 | 0 | 0 |
| ORF 2579 | 0 | 0 | 0 | 0 | 0 | 0 | 0 | 0 | 0 | 0 | 0 |
| ORF 2777 | 0 | 0 | 0 | 0 | 0 | 0 | 0 | 0 | 0 | 0 | 0 |
| ORF 2819 | 0 | 0 | 0 | 0 | 0 | 0 | 0 | 0 | 0 | 0 | 0 |
| ORF 2847 | 0 | 0 | 0 | 0 | 0 | 0 | 0 | 0 | 0 | 0 | 0 |
| ORF 2858 | 0 | 2 | 2 | 2 | 0 | 2 | 0 | 2 | 2 | 2 | 2 |
| ORF 3103 | 0 | 0 | 0 | 0 | 0 | 0 | 0 | 0 | 0 | 0 | 0 |
| ORF 3184 | 0 | 2 | 0 | 0 | 0 | 2 | 2 | 0 | 0 | 2 | 0 |
| ORF 3401 | 0 | 0 | 0 | 0 | 0 | 0 | 0 | 0 | 0 | 0 | 0 |
| ORF 3792 | 0 | 0 | 0 | 0 | 0 | 2 | 0 | 0 | 0 | 0 | 0 |
| ORF 3840 | 0 | 0 | 0 | 0 | 0 | 0 | 0 | 0 | 0 | 0 | 0 |
| ORF 4101 | 0 | 2 | 0 | 2 | 0 | 2 | 0 | 0 | 0 | 2 | 0 |
| ORF 660 | 0 | 0 | 0 | 0 | 0 | 0 | 0 | 0 | 0 | 0 | 0 |
| ORF 757 | 0 | 0 | 0 | 0 | 0 | 0 | 0 | 0 | 0 | 0 | 0 |
| ORF 799 | 0 | 0 | 0 | 0 | 0 | 0 | 0 | 0 | 0 | 0 | 0 |
| ORF 817 | 0 | 0 | 0 | 0 | 0 | 0 | 0 | 0 | 0 | 0 | 0 |

| Gene | CLIP61839-1/2a | CLIP42655-1/2b | CLIP3559-1/2b | CLIP46686-1/2b | CLIP989-1/2b | CLIP87878-1/2b | CLIP87879-1/2b | CLIP87888-1/2b | CLIP87897-1/2b | CLIP87909-1/2b |
|---|---|---|---|---|---|---|---|---|---|---|
| Lmo0017 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 |
| Lmo0066 | 2 | 0 | 2 | 2 | 2 | 0 | 0 | 0 | 0 | 0 |
| Lmo0067 | 2 | 0 | 0 | 2 | 2 | 0 | 0 | 0 | 0 | 0 |
| Lmo0068 | 2 | 0 | 2 | 2 | 2 | 0 | 0 | 2 | 0 | 0 |
| Lmo0069 | 0 | 0 | 2 | 2 | 2 | 0 | 2 | 2 | 2 | 0 |
| Lmo0071 | 0 | 2 | 2 | 2 | 2 | 2 | 2 | 2 | 2 | 0 |
| Lmo0072 | 2 | 2 | 2 | 2 | 2 | 2 | 2 | 2 | 2 | 0 |
| Lmo0079 | 2 | 2 | 2 | 2 | 2 | 2 | 2 | 2 | 2 | 2 |
| Lmo0080 | 2 | 2 | 2 | 2 | 2 | 2 | 2 | 2 | 0 | 2 |
| Lmo0081 | 2 | 0 | 2 | 0 | 0 | 0 | 2 | 0 | 0 | 0 |
| Lmo0082 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 |
| Lmo0106 | 2 | 0 | 2 | 0 | 0 | 0 | 2 | 0 | 0 | 2 |
| Lmo0141 | 2 | 0 | 0 | 2 | 2 | 0 | 2 | 0 | 0 | 2 |
| Lmo0143 | 0 | 0 | 0 | 0 | 0 | 0 | 0 | 0 | 0 | 2 |
| Lmo0144 | 0 | 0 | 0 | 2 | 2 | 0 | 2 | 0 | 0 | 2 |
| Lmo0147 | 2 | 2 | 0 | 2 | 2 | 0 | 2 | 0 | 0 | 0 |
| Lmo0151 | 0 | 0 | 0 | 0 | 0 | 0 | 0 | 0 | 0 | 0 |
| Lmo0171 | 2 | 0 | 0 | 0 | 0 | 0 | 0 | 0 | 0 | 0 |
| Lmo0172 | 2 | 0 | 0 | 0 | 0 | 0 | 0 | 0 | 0 | 0 |
| Lmo0252 | 0 | 0 | 0 | 0 | 0 | 0 | 0 | 0 | 0 | 0 |
| Lmo0253 | 0 | 0 | 0 | 0 | 0 | 0 | 0 | 0 | 0 | 0 |
| Lmo0304 | 0 | 2 | 2 | 0 | 0 | 2 | 2 | 2 | 2 | 2 |
| Lmo0310 | 0 | 0 | 2 | 0 | 0 | 0 | 0 | 0 | 0 | 2 |
| Lmo0320 | 2 | 2 | 2 | 2 | 2 | 2 | 2 | 2 | 2 | 2 |
| Lmo0333 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 |
| Lmo0338 | 0 | 0 | 0 | 0 | 0 | 0 | 0 | 0 | 0 | 0 |
| Lmo0378 | 2 | 2 | 0 | 2 | 2 | 2 | 2 | 2 | 2 | 2 |
| Lmo0379 | 2 | 0 | 0 | 0 | 0 | 0 | 0 | 0 | 0 | 0 |
| Lmo0380 | 2 | 2 | 0 | 2 | 2 | 2 | 2 | 2 | 2 | 2 |
| Lmo0381 | 2 | 2 | 0 | 2 | 2 | 2 | 2 | 2 | 2 | 2 |
| Lmo0409 | 2 | 2 | 0 | 0 | 2 | 0 | 0 | 0 | 2 | 0 |
| Lmo0419 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 |
| Lmo0444 | 2 | 2 | 0 | 0 | 0 | 2 | 2 | 2 | 2 | 2 |
| Lmo0448 | 2 | 2 | 2 | 0 | 2 | 2 | 2 | 2 | 2 | 2 |
| Lmo0459 | 0 | 2 | 2 | 2 | 2 | 2 | 2 | 2 | 2 | 2 |
| Lmo0462 | 0 | 2 | 2 | 2 | 2 | 2 | 2 | 2 | 2 | 2 |
| Lmo0466 | 0 | 0 | 0 | 0 | 0 | 0 | 0 | 0 | 0 | 0 |
| Lmo0467 | 0 | 0 | 0 | 0 | 0 | 0 | 0 | 0 | 0 | 0 |
| Lmo0471 | 0 | 0 | 0 | 0 | 0 | 0 | 0 | 0 | 0 | 0 |
| Lmo0472 | 2 | 0 | 0 | 2 | 2 | 0 | 0 | 2 | 2 | 2 |
| Lmo0473 | 0 | 0 | 0 | 0 | 0 | 0 | 2 | 0 | 0 | 2 |
| Lmo0474 | 2 | 0 | 0 | 0 | 0 | 0 | 2 | 0 | 0 | 0 |
| Lmo0493 | 2 | 2 | 2 | 2 | 2 | 2 | 2 | 2 | 2 | 2 |
| Lmo0497 | 2 | 2 | 2 | 2 | 2 | 2 | 2 | 2 | 2 | 2 |
| Lmo0525 | 2 | 0 | 0 | 0 | 0 | 0 | 0 | 0 | 0 | 0 |
| Lmo0549 | 2 | 2 | 2 | 2 | 2 | 2 | 2 | 2 | 2 | 2 |
| Lmo0630 | 2 | 2 | 2 | 2 | 2 | 2 | 2 | 2 | 0 | 2 |
| Lmo0631 | 2 | 2 | 2 | 2 | 2 | 2 | 2 | 2 | 2 | 2 |
| Lmo0632 | 2 | 2 | 2 | 2 | 2 | 2 | 2 | 2 | 2 | 2 |
| Lmo0732 | 2 | 2 | 2 | 2 | 2 | 2 | 2 | 2 | 0 | 0 |
| Lmo0733 | 2 | 2 | 2 | 2 | 2 | 2 | 2 | 2 | 2 | 2 |
| Lmo0734 | 2 | 0 | 0 | 0 | 0 | 0 | 0 | 0 | 0 | 0 |
| Lmo0735 | 2 | 0 | 0 | 0 | 0 | 0 | 0 | 0 | 0 | 0 |
| Lmo0739 | 2 | 0 | 0 | 0 | 0 | 0 | 0 | 0 | 0 | 0 |
| Lmo0750 | 2 | 0 | 0 | 0 | 0 | 0 | 0 | 0 | 0 | 0 |
| Lmo0751 | 2 | 2 | 0 | 0 | 0 | 2 | 2 | 2 | 2 | 0 |
| Lmo0752 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 |
| Lmo0780 | 2 | 0 | 0 | 0 | 0 | 0 | 0 | 0 | 0 | 0 |
| Lmo0801 | 2 | 0 | 0 | 0 | 0 | 0 | 0 | 0 | 0 | 0 |
| Lmo0804 | 0 | 0 | 0 | 2 | 2 | 2 | 2 | 2 | 0 | 2 |
| Lmo0832 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 |
| Lmo0842 | 2 | 2 | 2 | 2 | 2 | 2 | 2 | 2 | 2 | 2 |
| Lmo1030 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 |
| Lmo1060 | 2 | 0 | 0 | 0 | 0 | 0 | 0 | 0 | 0 | 0 |
| Lmo1061 | 2 | 0 | 0 | 0 | 0 | 0 | 0 | 0 | 0 | 0 |
| Lmo1063 | 2 | 0 | 0 | 0 | 0 | 0 | 0 | 0 | 0 | 0 |
| Lmo1076 | 2 | 2 | 2 | 2 | 2 | 2 | 2 | 2 | 2 | 2 |
| Lmo1077 | 2 | 2 | 2 | 2 | 2 | 2 | 2 | 2 | 2 | 2 |
| Lmo1080 | 2 | 2 | 2 | 2 | 2 | 2 | 2 | 2 | 2 | 2 |
| Lmo1081 | 2 | 2 | 2 | 2 | 2 | 2 | 2 | 2 | 2 | 2 |
| Lmo1082 | 2 | 2 | 2 | 2 | 2 | 2 | 2 | 2 | 2 | 2 |
| Lmo1085 | 2 | 2 | 2 | 2 | 2 | 2 | 2 | 2 | 2 | 2 |
| Lmo1088 | 2 | 2 | 2 | 2 | 2 | 2 | 2 | 2 | 2 | 2 |
| Lmo1090 | 2 | 2 | 2 | 2 | 2 | 2 | 2 | 2 | 2 | 2 |
| Lmo1091 | 2 | 2 | 2 | 2 | 2 | 2 | 2 | 2 | 2 | 2 |
| Lmo1102 | 2 | 2 | 2 | 2 | 2 | 0 | 0 | 0 | 2 | 0 |
| Lmo1115 | 2 | 0 | 0 | 2 | 2 | 0 | 0 | 0 | 0 | 0 |
| Lmo1116 | 0 | 2 | 0 | 0 | 0 | 2 | 2 | 2 | 2 | 0 |
| Lmo1118 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 |
| Lmo1120 | 0 | 2 | 2 | 2 | 2 | 2 | 2 | 2 | 2 | 0 |
| Lmo1133 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 |
| Lmo1307 | 2 | 0 | 0 | 0 | 0 | 0 | 0 | 0 | 0 | 0 |
| Lmo1441 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 |
| Lmo1597 | 2 | 2 | 2 | 2 | 2 | 2 | 0 | 2 | 2 | 0 |
| Lmo1604 | 2 | 2 | 2 | 2 | 2 | 0 | 0 | 2 | 2 | 2 |
| Lmo1648 | 2 | 2 | 0 | 2 | 0 | 0 | 0 | 0 | 0 | 0 |
| Lmo1666 | 2 | 2 | 2 | 2 | 2 | 2 | 2 | 2 | 2 | 2 |
| Lmo1714 | 0 | 2 | 2 | 2 | 2 | 2 | 2 | 2 | 2 | 2 |
| Lmo1968 | 2 | 0 | 0 | 0 | 0 | 0 | 0 | 0 | 0 | 0 |
| Lmo1973 | 2 | 0 | 0 | 0 | 0 | 0 | 0 | 0 | 0 | 0 |
| Lmo1974 | 2 | 0 | 0 | 0 | 0 | 0 | 0 | 0 | 0 | 0 |
| Lmo2026 | 2 | 0 | 0 | 0 | 0 | 0 | 0 | 0 | 0 | 0 |
| Lmo2027 | 2 | 2 | 2 | 2 | 2 | 2 | 2 | 2 | 0 | 2 |
| Lmo2276 | 2 | 2 | 2 | 2 | 2 | 2 | 0 | 0 | 0 | 2 |
| Lmo2277 | 2 | 0 | 0 | 0 | 0 | 0 | 0 | 0 | 0 | 0 |
| Lmo2311 | 2 | 0 | 0 | 0 | 0 | 0 | 0 | 0 | 0 | 0 |
| Lmo2312 | 2 | 2 | 2 | 2 | 2 | 2 | 2 | 2 | 0 | 2 |
| Lmo2324 | 2 | 0 | 0 | 2 | 2 | 2 | 2 | 0 | 0 | 0 |
| Lmo2408 | 2 | 0 | 0 | 0 | 0 | 0 | 0 | 0 | 0 | 0 |
| Lmo2443 | 2 | 2 | 2 | 2 | 2 | 2 | 2 | 2 | 0 | 2 |
| Lmo2576 | 2 | 0 | 0 | 0 | 0 | 0 | 0 | 0 | 0 | 0 |
| Lmo2594 | 2 | 2 | 2 | 0 | 2 | 2 | 2 | 0 | 0 | 2 |
| Lmo2595 | 0 | 0 | 2 | 0 | 0 | 0 | 2 | 0 | 0 | 2 |
| Lmo2671 | 2 | 2 | 2 | 2 | 2 | 2 | 2 | 2 | 2 | 2 |
| Lmo2686 | 2 | 0 | 0 | 0 | 0 | 0 | 0 | 0 | 0 | 2 |
| Lmo2732 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 |
| Lmo2786 | 2 | 0 | 0 | 0 | 0 | 0 | 0 | 0 | 0 | 0 |
| Lmo2787 | 2 | 0 | 0 | 0 | 0 | 0 | 0 | 0 | 0 | 0 |
| Lmo2807 | 2 | 0 | 0 | 0 | 0 | 0 | 0 | 2 | 0 | 0 |
| Lmo2809 | 2 | 2 | 2 | 2 | 2 | 2 | 2 | 2 | 2 | 2 |
| Lmo2821 | 2 | 2 | 2 | 2 | 2 | 2 | 2 | 2 | 2 | 2 |
| Lin0060 | 2 | 0 | 0 | 2 | 2 | 0 | 0 | 0 | 0 | 2 |
| Lin0197 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 |
| Lin0415 | 2 | 2 | 2 | 2 | 2 | 2 | 0 | 2 | 0 | 2 |
| Lin0453 | 0 | 0 | 0 | 0 | 0 | 0 | 0 | 0 | 0 | 0 |
| Lin0454 | 0 | 0 | 0 | 0 | 0 | 0 | 0 | 0 | 0 | 0 |
| Lin0455 | 0 | 0 | 0 | 0 | 0 | 0 | 0 | 0 | 0 | 0 |
| Lin0465 | 0 | 0 | 0 | 0 | 0 | 0 | 0 | 0 | 0 | 0 |
| Lin0476 | 2 | 2 | 2 | 2 | 2 | 0 | 0 | 0 | 0 | 0 |
| Lin0661 | 2 | 0 | 0 | 0 | 0 | 0 | 0 | 0 | 0 | 0 |
| Lin0665 | 2 | 2 | 2 | 2 | 2 | 2 | 2 | 2 | 2 | 2 |
| Lin0677 | 0 | 0 | 0 | 0 | 0 | 0 | 0 | 0 | 0 | 0 |
| Lin0739 | 0 | 0 | 0 | 0 | 0 | 0 | 0 | 0 | 0 | 0 |
| Lin0740 | 2 | 2 | 2 | 2 | 2 | 0 | 0 | 0 | 0 | 0 |
| Lin0803 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 |
| Lin1066 | 0 | 0 | 0 | 0 | 0 | 0 | 0 | 0 | 0 | 0 |
| Lin1069 | 0 | 0 | 0 | 0 | 0 | 0 | 0 | 0 | 0 | 0 |
| Lin1073 | 0 | 0 | 0 | 0 | 0 | 0 | 0 | 0 | 0 | 0 |
| Lin1083 | 0 | 0 | 2 | 2 | 2 | 0 | 0 | 0 | 0 | 0 |
| Lin1084 | 0 | 2 | 2 | 2 | 2 | 2 | 2 | 2 | 2 | 2 |
| Lin1090 | 0 | 2 | 2 | 2 | 2 | 2 | 2 | 2 | 2 | 2 |
| Lin1204 | 0 | 2 | 2 | 2 | 2 | 2 | 2 | 0 | 2 | 2 |
| Lin1262 | 2 | 2 | 2 | 2 | 2 | 0 | 0 | 0 | 0 | 0 |
| Lin1299 | 2 | 2 | 2 | 2 | 2 | 2 | 0 | 0 | 2 | 0 |
| Lin1328 | 2 | 2 | 2 | 2 | 2 | 0 | 0 | 0 | 0 | 0 |
| Lin1452 | 0 | 0 | 0 | 0 | 0 | 0 | 0 | 0 | 0 | 0 |
| Lin1621 | 0 | 0 | 0 | 0 | 0 | 0 | 0 | 0 | 0 | 0 |
| Lin2397 | 2 | 0 | 0 | 2 | 2 | 0 | 2 | 0 | 0 | 2 |
| Lin2494 | 2 | 2 | 2 | 2 | 2 | 2 | 2 | 2 | 2 | 2 |
| Lin2603 | 0 | 0 | 2 | 2 | 0 | 0 | 0 | 0 | 0 | 0 |
| Lin2724 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 |
| Lin2743 | 2 | 2 | 0 | 2 | 2 | 2 | 0 | 2 | 2 | 0 |
| Lin2744 | 2 | 2 | 2 | 2 | 2 | 2 | 0 | 2 | 2 | 0 |
| Lin2839 | 0 | 0 | 2 | 0 | 0 | 0 | 0 | 0 | 0 | 2 |
| Lin2918 | 0 | 0 | 0 | 0 | 0 | 0 | 0 | 0 | 0 | 0 |
| ORF 105 | 0 | 2 | 2 | 0 | 0 | 2 | 0 | 2 | 2 | 0 |
| ORF 1390 | 0 | 0 | 0 | 0 | 0 | 0 | 0 | 0 | 0 | 0 |
| ORF 1445 | 0 | 0 | 0 | 0 | 0 | 0 | 0 | 0 | 0 | 0 |
| ORF 1490 | 0 | 2 | 2 | 2 | 2 | 2 | 2 | 2 | 2 | 2 |
| ORF 1491 | 0 | 2 | 2 | 2 | 2 | 2 | 2 | 2 | 2 | 2 |
| ORF 1590 | 2 | 2 | 2 | 2 | 2 | 2 | 2 | 2 | 2 | 2 |
| ORF 1761 | 0 | 2 | 2 | 2 | 2 | 2 | 2 | 2 | 2 | 2 |
| ORF 2017 | 0 | 2 | 2 | 2 | 2 | 2 | 2 | 2 | 2 | 2 |
| ORF 2074 | 2 | 2 | 2 | 2 | 2 | 2 | 2 | 2 | 2 | 2 |
| ORF 2076 | 2 | 2 | 2 | 2 | 2 | 2 | 2 | 2 | 2 | 2 |
| ORF 2110 | 0 | 0 | 0 | 0 | 0 | 0 | 0 | 0 | 0 | 0 |
| ORF 2372 | 0 | 0 | 0 | 0 | 0 | 0 | 0 | 0 | 0 | 0 |
| ORF 2541 | 0 | 2 | 2 | 2 | 2 | 2 | 2 | 2 | 2 | 2 |
| ORF 2579 | 2 | 2 | 2 | 2 | 2 | 2 | 2 | 2 | 2 | 2 |
| ORF 2777 | 0 | 2 | 2 | 2 | 2 | 2 | 2 | 2 | 2 | 2 |
| ORF 2819 | 0 | 2 | 2 | 2 | 2 | 2 | 2 | 2 | 2 | 2 |
| ORF 2847 | 0 | 0 | 0 | 0 | 0 | 0 | 0 | 0 | 0 | 0 |
| ORF 2858 | 2 | 2 | 2 | 2 | 2 | 2 | 2 | 2 | 2 | 0 |
| ORF 3103 | 0 | 2 | 0 | 0 | 0 | 2 | 0 | 2 | 2 | 0 |
| ORF 3184 | 0 | 2 | 0 | 0 | 2 | 2 | 2 | 2 | 2 | 0 |
| ORF 3401 | 0 | 2 | 0 | 0 | 2 | 0 | 0 | 0 | 0 | 0 |
| ORF 3792 | 0 | 2 | 2 | 2 | 2 | 2 | 2 | 2 | 2 | 2 |
| ORF 3840 | 0 | 2 | 2 | 2 | 2 | 2 | 2 | 2 | 2 | 2 |
| ORF 4101 | 2 | 2 | 2 | 0 | 2 | 2 | 2 | 2 | 2 | 2 |
| ORF 660 | 0 | 0 | 0 | 0 | 0 | 0 | 2 | 0 | 0 | 0 |
| ORF 757 | 0 | 0 | 2 | 0 | 0 | 0 | 2 | 0 | 0 | 0 |
| ORF 799 | 0 | 0 | 0 | 0 | 0 | 0 | 0 | 0 | 0 | 0 |
| ORF 817 | 0 | 2 | 2 | 2 | 2 | 2 | 2 | 2 | 2 | 0 |

| Gene | CLIP87910-1/2b | CLIP14842-1/2b | CLIP70086-1/2b | CLIP70848-1/2b | CLIP76278-1/2b | CLIP89460-1/2b | CLIP74906-3b |
|---|---|---|---|---|---|---|---|
| Lmo0017 | 2 | 2 | 2 | 2 | 2 | 2 | 2 |
| Lmo0036 | 2 | 2 | 2 | 2 | 2 | 2 | 2 |
| Lmo0037 | 2 | 2 | 2 | 2 | 2 | 2 | 2 |
| Lmo0066 | 0 | 0 | 0 | 0 | 0 | 0 | 0 |
| Lmo0067 | 0 | 0 | 0 | 0 | 0 | 0 | 0 |
| Lmo0068 | 2 | 0 | 2 | 0 | 2 | 2 | 0 |
| Lmo0069 | 2 | 2 | 2 | 2 | 2 | 2 | 2 |
| Lmo0071 | 2 | 2 | 2 | 2 | 2 | 2 | 2 |
| Lmo0072 | 2 | 2 | 2 | 2 | 2 | 2 | 2 |
| Lmo0079 | 2 | 2 | 2 | 2 | 2 | 2 | 2 |
| Lmo0080 | 2 | 2 | 2 | 2 | 2 | 2 | 2 |
| Lmo0081 | 2 | 0 | 2 | 0 | 2 | 2 | 0 |
| Lmo0082 | 2 | 2 | 2 | 2 | 2 | 2 | 0 |
| Lmo0083 | 2 | 2 | 2 | 2 | 2 | 2 | 2 |
| Lmo0094 | 2 | 2 | 2 | 2 | 2 | 2 | 2 |
| Lmo0106 | 0 | 0 | 2 | 0 | 2 | 2 | 0 |
| Lmo0141 | 0 | 0 | 0 | 0 | 0 | 0 | 0 |
| Lmo0143 | 0 | 0 | 0 | 0 | 0 | 0 | 0 |
| Lmo0144 | 0 | 0 | 0 | 0 | 0 | 0 | 0 |
| Lmo0147 | 0 | 2 | 2 | 2 | 2 | 2 | 2 |
| Lmo0151 | 0 | 0 | 0 | 0 | 0 | 0 | 0 |
| Lmo0171 | 0 | 0 | 0 | 0 | 0 | 0 | 0 |
| Lmo0172 | 0 | 0 | 0 | 0 | 0 | 0 | 0 |
| Lmo0252 | 0 | 0 | 0 | 0 | 0 | 0 | 0 |
| Lmo0253 | 0 | 0 | 0 | 0 | 0 | 0 | 0 |
| Lmo0304 | 2 | 2 | 2 | 2 | 2 | 2 | 2 |
| Lmo0310 | 2 | 0 | 2 | 0 | 2 | 2 | 0 |
| Lmo0320 | 2 | 2 | 0 | 2 | 2 | 2 | 2 |
| Lmo0333 | 2 | 2 | 2 | 2 | 2 | 2 | 2 |
| Lmo0334 | 2 | 2 | 2 | 2 | 2 | 2 | 2 |
| Lmo0338 | 0 | 0 | 0 | 0 | 0 | 0 | 0 |
| Lmo0378 | 0 | 2 | 0 | 2 | 0 | 0 | 2 |
| Lmo0379 | 0 | 0 | 0 | 0 | 0 | 0 | 0 |
| Lmo0380 | 0 | 2 | 0 | 2 | 0 | 0 | 2 |
| Lmo0381 | 0 | 2 | 0 | 2 | 0 | 0 | 2 |
| Lmo0409 | 0 | 2 | 2 | 2 | 2 | 2 | 2 |
| Lmo0419 | 2 | 2 | 2 | 2 | 2 | 2 | 2 |
| Lmo0440 | 2 | 2 | 2 | 2 | 2 | 2 | 2 |
| Lmo0444 | 0 | 2 | 0 | 2 | 0 | 0 | 2 |
| Lmo0448 | 2 | 2 | 2 | 2 | 2 | 2 | 2 |
| Lmo0459 | 2 | 2 | 2 | 2 | 2 | 2 | 2 |
| Lmo0462 | 2 | 2 | 2 | 2 | 2 | 2 | 2 |
| Lmo0466 | 0 | 0 | 0 | 0 | 0 | 0 | 0 |
| Lmo0467 | 0 | 0 | 0 | 0 | 0 | 0 | 0 |
| Lmo0471 | 0 | 0 | 0 | 0 | 0 | 0 | 0 |
| Lmo0472 | 0 | 0 | 0 | 0 | 0 | 0 | 0 |
| Lmo0473 | 0 | 0 | 0 | 0 | 2 | 2 | 0 |
| Lmo0474 | 0 | 0 | 0 | 0 | 0 | 0 | 0 |
| Lmo0493 | 2 | 2 | 2 | 2 | 2 | 2 | 2 |
| Lmo0497 | 2 | 2 | 2 | 2 | 2 | 2 | 2 |
| Lmo0525 | 0 | 0 | 0 | 0 | 0 | 0 | 0 |
| Lmo0549 | 2 | 2 | 2 | 2 | 2 | 2 | 2 |
| Lmo0630 | 2 | 2 | 2 | 2 | 2 | 2 | 2 |
| Lmo0631 | 2 | 2 | 2 | 2 | 2 | 2 | 2 |
| Lmo0632 | 2 | 2 | 2 | 2 | 2 | 2 | 2 |
| Lmo0732 | 2 | 2 | 2 | 2 | 2 | 2 | 0 |
| Lmo0733 | 2 | 2 | 2 | 2 | 2 | 2 | 2 |
| Lmo0734 | 0 | 0 | 0 | 0 | 0 | 0 | 0 |
| Lmo0735 | 0 | 0 | 0 | 0 | 0 | 0 | 0 |
| Lmo0739 | 0 | 0 | 0 | 0 | 0 | 0 | 0 |
| Lmo0750 | 0 | 0 | 0 | 0 | 0 | 0 | 0 |
| Lmo0751 | 0 | 2 | 0 | 2 | 0 | 0 | 2 |
| Lmo0752 | 2 | 2 | 2 | 2 | 2 | 2 | 2 |
| Lmo0753 | 2 | 2 | 2 | 2 | 2 | 2 | 2 |
| Lmo0780 | 0 | 0 | 2 | 0 | 0 | 0 | 0 |
| Lmo0801 | 0 | 0 | 0 | 0 | 0 | 0 | 0 |
| Lmo0804 | 2 | 0 | 0 | 0 | 0 | 0 | 0 |
| Lmo0832 | 2 | 2 | 2 | 2 | 2 | 2 | 2 |
| Lmo0833 | 2 | 2 | 2 | 2 | 2 | 2 | 2 |
| Lmo0834 | 2 | 2 | 2 | 2 | 2 | 2 | 2 |
| Lmo0835 | 2 | 2 | 2 | 2 | 2 | 2 | 2 |
| Lmo0842 | 2 | 2 | 2 | 2 | 2 | 2 | 2 |
| Lmo1030 | 2 | 2 | 2 | 2 | 2 | 2 | 2 |
| Lmo1036 | 2 | 2 | 2 | 2 | 2 | 2 | 2 |
| Lmo1060 | 0 | 0 | 0 | 0 | 0 | 0 | 0 |
| Lmo1061 | 0 | 0 | 0 | 0 | 0 | 0 | 0 |
| Lmo1063 | 0 | 0 | 0 | 0 | 0 | 0 | 0 |
| Lmo1076 | 2 | 2 | 2 | 2 | 2 | 2 | 2 |
| Lmo1077 | 2 | 2 | 2 | 2 | 2 | 2 | 2 |
| Lmo1080 | 2 | 2 | 2 | 2 | 2 | 2 | 2 |
| Lmo1081 | 2 | 2 | 2 | 2 | 2 | 2 | 2 |
| Lmo1082 | 2 | 2 | 2 | 2 | 2 | 2 | 2 |
| Lmo1085 | 2 | 2 | 2 | 2 | 2 | 2 | 2 |
| Lmo1088 | 2 | 2 | 2 | 2 | 2 | 2 | 2 |
| Lmo1090 | 2 | 2 | 2 | 2 | 2 | 2 | 2 |
| Lmo1091 | 2 | 2 | 2 | 2 | 2 | 2 | 2 |
| Lmo1102 | 2 | 2 | 2 | 2 | 2 | 2 | 0 |
| Lmo1115 | 0 | 0 | 0 | 0 | 0 | 0 | 0 |
| Lmo1116 | 0 | 2 | 0 | 2 | 0 | 0 | 2 |
| Lmo1118 | 0 | 0 | 0 | 0 | 0 | 0 | 0 |
| Lmo1119 | 0 | 0 | 0 | 0 | 0 | 0 | 0 |
| Lmo1120 | 2 | 2 | 2 | 2 | 2 | 2 | 2 |
| Lmo1133 | 2 | 2 | 2 | 2 | 2 | 2 | 2 |
| Lmo1135 | 2 | 2 | 2 | 2 | 2 | 2 | 2 |
| Lmo1307 | 0 | 0 | 0 | 0 | 0 | 0 | 0 |
| Lmo1441 | 2 | 2 | 2 | 2 | 2 | 2 | 2 |
| Lmo1451 | 2 | 2 | 2 | 2 | 2 | 2 | 2 |
| Lmo1597 | 2 | 2 | 2 | 2 | 2 | 2 | 2 |
| Lmo1604 | 2 | 2 | 2 | 2 | 2 | 2 | 0 |
| Lmo1648 | 0 | 0 | 0 | 2 | 0 | 0 | 2 |
| Lmo1666 | 2 | 2 | 2 | 2 | 2 | 2 | 2 |
| Lmo1714 | 2 | 2 | 2 | 2 | 2 | 2 | 2 |
| Lmo1968 | 0 | 0 | 0 | 0 | 0 | 0 | 0 |
| Lmo1973 | 0 | 0 | 0 | 0 | 0 | 0 | 0 |
| Lmo1974 | 0 | 0 | 0 | 0 | 0 | 0 | 0 |
| Lmo2026 | 0 | 0 | 0 | 0 | 0 | 0 | 0 |
| Lmo2027 | 2 | 2 | 2 | 2 | 2 | 2 | 0 |
| Lmo2276 | 2 | 2 | 2 | 2 | 2 | 2 | 0 |
| Lmo2277 | 0 | 0 | 0 | 0 | 0 | 0 | 0 |
| Lmo2311 | 0 | 0 | 0 | 0 | 0 | 2 | 0 |
| Lmo2312 | 2 | 0 | 2 | 0 | 2 | 2 | 2 |
| Lmo2324 | 2 | 0 | 2 | 0 | 0 | 0 | 0 |
| Lmo2408 | 0 | 0 | 0 | 0 | 0 | 0 | 0 |
| Lmo2443 | 2 | 2 | 2 | 2 | 2 | 2 | 2 |
| Lmo2576 | 0 | 0 | 0 | 0 | 0 | 0 | 0 |
| Lmo2594 | 2 | 2 | 2 | 2 | 2 | 2 | 0 |
| Lmo2595 | 2 | 0 | 2 | 0 | 2 | 2 | 0 |
| Lmo2671 | 2 | 2 | 2 | 2 | 2 | 2 | 2 |
| Lmo2686 | 0 | 0 | 0 | 0 | 0 | 0 | 0 |
| Lmo2732 | 2 | 2 | 2 | 2 | 2 | 2 | 2 |
| Lmo2736 | 2 | 2 | 2 | 2 | 2 | 2 | 2 |
| Lmo2786 | 0 | 0 | 0 | 0 | 0 | 0 | 0 |
| Lmo2787 | 0 | 0 | 0 | 0 | 0 | 0 | 0 |
| Lmo2807 | 0 | 0 | 0 | 0 | 0 | 0 | 0 |
| Lmo2809 | 2 | 2 | 2 | 2 | 2 | 2 | 2 |
| Lmo2821 | 2 | 2 | 2 | 2 | 2 | 2 | 2 |
| Lin0060 | 0 | 0 | 0 | 0 | 0 | 0 | 0 |
| Lin0197 | 0 | 0 | 0 | 0 | 0 | 0 | 0 |
| Lin0332 | 0 | 0 | 0 | 0 | 0 | 0 | 0 |
| Lin0397 | 0 | 0 | 0 | 0 | 0 | 0 | 0 |
| Lin0415 | 2 | 2 | 2 | 2 | 2 | 2 | 0 |
| Lin0453 | 0 | 0 | 0 | 0 | 0 | 0 | 0 |
| Lin0454 | 0 | 0 | 0 | 0 | 0 | 0 | 0 |
| Lin0455 | 0 | 0 | 0 | 0 | 0 | 0 | 0 |
| Lin0465 | 0 | 0 | 0 | 0 | 0 | 0 | 0 |
| Lin0476 | 0 | 0 | 2 | 0 | 2 | 2 | 0 |
| Lin0661 | 0 | 0 | 0 | 0 | 0 | 2 | 0 |
| Lin0665 | 2 | 2 | 2 | 2 | 2 | 2 | 2 |
| Lin0677 | 0 | 0 | 0 | 0 | 0 | 0 | 0 |
| Lin0739 | 0 | 0 | 0 | 0 | 0 | 0 | 0 |
| Lin0740 | 0 | 2 | 2 | 2 | 0 | 2 | 0 |
| Lin0803 | 0 | 0 | 0 | 0 | 0 | 0 | 0 |
| Lin0868 | 0 | 0 | 0 | 0 | 0 | 0 | 0 |
| Lin1066 | 0 | 0 | 0 | 0 | 0 | 0 | 0 |
| Lin1069 | 0 | 0 | 0 | 0 | 0 | 0 | 0 |
| Lin1073 | 0 | 0 | 0 | 0 | 0 | 0 | 0 |
| Lin1083 | 2 | 0 | 2 | 0 | 2 | 2 | 0 |
| Lin1084 | 2 | 2 | 2 | 2 | 2 | 2 | 2 |
| Lin1090 | 2 | 2 | 2 | 2 | 2 | 2 | 2 |
| Lin1204 | 0 | 2 | 2 | 2 | 2 | 2 | 2 |
| Lin1262 | 0 | 2 | 2 | 2 | 2 | 2 | 0 |
| Lin1299 | 2 | 2 | 2 | 2 | 2 | 2 | 2 |
| Lin1328 | 0 | 2 | 2 | 2 | 2 | 2 | 0 |
| Lin1452 | 0 | 0 | 0 | 0 | 0 | 0 | 0 |
| Lin1621 | 0 | 0 | 0 | 0 | 0 | 0 | 0 |
| Lin2397 | 0 | 0 | 0 | 0 | 2 | 0 | 0 |
| Lin2494 | 2 | 2 | 2 | 2 | 2 | 2 | 2 |
| Lin2603 | 2 | 0 | 0 | 0 | 0 | 2 | 0 |
| Lin2724 | 0 | 0 | 0 | 0 | 0 | 0 | 0 |
| Lin2741 | 0 | 0 | 0 | 0 | 0 | 0 | 0 |
| Lin2743 | 0 | 2 | 2 | 2 | 0 | 2 | 2 |
| Lin2744 | 0 | 2 | 2 | 2 | 0 | 2 | 2 |
| Lin2839 | 0 | 0 | 2 | 0 | 2 | 2 | 0 |
| Lin2918 | 0 | 0 | 0 | 0 | 0 | 0 | 0 |
| ORF 105 | 2 | 2 | 2 | 2 | 2 | 2 | 2 |
| ORF 1390 | 0 | 0 | 0 | 2 | 0 | 2 | 0 |
| ORF 1445 | 0 | 0 | 0 | 0 | 0 | 0 | 0 |
| ORF 1490 | 2 | 2 | 2 | 2 | 2 | 2 | 2 |
| ORF 1491 | 2 | 2 | 2 | 2 | 2 | 2 | 2 |
| ORF 1590 | 0 | 2 | 2 | 2 | 2 | 2 | 2 |
| ORF 1761 | 2 | 2 | 2 | 2 | 2 | 2 | 2 |
| ORF 2017 | 2 | 2 | 2 | 2 | 2 | 2 | 2 |
| ORF 2074 | 2 | 2 | 2 | 2 | 2 | 2 | 2 |
| ORF 2076 | 2 | 2 | 2 | 2 | 2 | 2 | 2 |
| ORF 2110 | 0 | 0 | 0 | 0 | 0 | 0 | 0 |
| ORF 2372 | 0 | 0 | 0 | 0 | 0 | 0 | 0 |
| ORF 2541 | 2 | 2 | 2 | 2 | 2 | 2 | 2 |
| ORF 2579 | 0 | 2 | 2 | 2 | 2 | 2 | 2 |
| ORF 2777 | 2 | 2 | 2 | 2 | 2 | 2 | 2 |
| ORF 2819 | 2 | 2 | 2 | 2 | 2 | 2 | 2 |
| ORF 2847 | 0 | 0 | 0 | 0 | 0 | 0 | 0 |
| ORF 2858 | 0 | 2 | 2 | 2 | 2 | 2 | 2 |
| ORF 3103 | 0 | 2 | 0 | 2 | 0 | 0 | 2 |
| ORF 3184 | 0 | 2 | 0 | 2 | 0 | 2 | 2 |
| ORF 3401 | 0 | 2 | 2 | 2 | 2 | 2 | 0 |
| ORF 3792 | 2 | 2 | 2 | 2 | 2 | 2 | 2 |
| ORF 3840 | 2 | 2 | 2 | 2 | 2 | 2 | 2 |
| ORF 4101 | 2 | 2 | 2 | 2 | 2 | 2 | 2 |
| ORF 660 | 0 | 0 | 0 | 0 | 0 | 0 | 0 |
| ORF 757 | 2 | 0 | 2 | 0 | 2 | 2 | 0 |
| ORF 799 | 0 | 0 | 0 | 0 | 0 | 0 | 0 |
| ORF 817 | 2 | 2 | 2 | 2 | 2 | 2 | 2 |

| | | | | | CLIP42635- | | | | | | |
|---|---|---|---|---|---|---|---|---|---|---|---|
| Gene | CLIP87900-3b | CLIP3558-3b | CLIP74917-7 | CLIP61673-7 | 4b Epi | CLIP42646-4b Epi | CLIP46679-4b | CLIP2698-4b | CLIP27993-4b | CLIP4598-4b | CLIP46684-4b |
| Lmo0017 | 2 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 | 2 |
| Lmo0066 | 0 | 0 | 0 | 0 | 0 | 0 | 0 | 0 | 0 | 0 | 0 |
| Lmo0067 | 0 | 0 | 0 | 0 | 0 | 0 | 0 | 0 | 0 | 0 | 0 |
| Lmo0068 | 2 | 0 | 0 | 0 | 2 | 2 | 2 | 0 | 2 | 2 | 2 |
| Lmo0069 | 0 | 2 | 2 | 2 | 2 | 2 | 2 | 0 | 2 | 2 | 2 |
| Lmo0071 | 2 | 2 | 2 | 2 | 2 | 2 | 2 | 0 | 2 | 2 | 2 |
| Lmo0072 | 2 | 2 | 2 | 0 | 2 | 2 | 2 | 2 | 2 | 2 | 2 |
| Lmo0079 | 2 | 2 | 2 | 2 | 2 | 2 | 2 | 2 | 2 | 2 | 2 |
| Lmo0080 | 2 | 2 | 2 | 2 | 2 | 2 | 2 | 2 | 2 | 2 | 2 |
| Lmo0081 | 0 | 0 | 0 | 2 | 0 | 0 | 0 | 0 | 0 | 0 | 0 |
| Lmo0082 | 2 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 | 2 |
| Lmo0106 | 0 | 0 | 0 | 2 | 2 | 2 | 2 | 2 | 2 | 2 | 2 |
| Lmo0141 | 0 | 0 | 0 | 2 | 2 | 2 | 2 | 2 | 2 | 2 | 2 |
| Lmo0143 | 0 | 0 | 0 | 0 | 0 | 0 | 0 | 0 | 0 | 0 | 0 |
| Lmo0144 | 0 | 0 | 0 | 2 | 2 | 2 | 2 | 2 | 2 | 2 | 2 |
| Lmo0147 | 2 | 2 | 0 | 2 | 2 | 2 | 2 | 2 | 2 | 2 | 2 |
| Lmo0151 | 0 | 0 | 0 | 0 | 0 | 0 | 0 | 0 | 0 | 0 | 0 |
| Lmo0171 | 0 | 0 | 0 | 0 | 0 | 0 | 0 | 0 | 0 | 0 | 0 |
| Lmo0172 | 0 | 0 | 0 | 0 | 0 | 0 | 0 | 0 | 0 | 0 | 0 |
| Lmo0252 | 0 | 0 | 0 | 0 | 0 | 0 | 0 | 0 | 0 | 0 | 0 |
| Lmo0253 | 0 | 0 | 0 | 0 | 0 | 0 | 0 | 0 | 0 | 0 | 0 |
| Lmo0304 | 2 | 2 | 2 | 2 | 0 | 0 | 0 | 0 | 0 | 0 | 0 |
| Lmo0310 | 0 | 0 | 0 | 0 | 0 | 0 | 0 | 0 | 0 | 0 | 0 |
| Lmo0320 | 2 | 2 | 2 | 0 | 2 | 2 | 2 | 2 | 2 | 2 | 2 |
| Lmo0333 | 2 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 | 2 |
| Lmo0338 | 0 | 0 | 0 | 0 | 0 | 0 | 0 | 0 | 0 | 0 | 0 |
| Lmo0378 | 2 | 2 | 2 | 2 | 0 | 0 | 0 | 0 | 0 | 0 | 0 |
| Lmo0379 | 0 | 0 | 0 | 0 | 0 | 0 | 0 | 0 | 0 | 0 | 0 |
| Lmo0380 | 2 | 2 | 2 | 2 | 0 | 0 | 0 | 0 | 0 | 0 | 0 |
| Lmo0381 | 2 | 2 | 2 | 2 | 0 | 0 | 0 | 0 | 0 | 0 | 0 |
| Lmo0409 | 0 | 2 | 2 | 2 | 2 | 2 | 2 | 2 | 0 | 0 | 0 |
| Lmo0419 | 2 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 | 2 |
| Lmo0444 | 2 | 2 | 2 | 2 | 0 | 0 | 0 | 0 | 0 | 0 | 0 |
| Lmo0448 | 2 | 2 | 2 | 2 | 2 | 2 | 2 | 2 | 2 | 0 | 2 |
| Lmo0459 | 2 | 2 | 2 | 2 | 2 | 2 | 2 | 2 | 2 | 2 | 2 |
| Lmo0462 | 2 | 2 | 2 | 2 | 2 | 2 | 2 | 2 | 2 | 2 | 2 |
| Lmo0466 | 0 | 0 | 0 | 0 | 0 | 0 | 0 | 0 | 0 | 0 | 0 |
| Lmo0467 | 0 | 0 | 0 | 0 | 0 | 0 | 0 | 0 | 0 | 0 | 0 |
| Lmo0471 | 0 | 0 | 0 | 0 | 0 | 0 | 0 | 0 | 0 | 0 | 0 |
| Lmo0472 | 0 | 0 | 0 | 0 | 0 | 0 | 0 | 0 | 0 | 0 | 0 |
| Lmo0473 | 0 | 0 | 0 | 0 | 2 | 2 | 2 | 0 | 2 | 0 | 0 |
| Lmo0474 | 0 | 0 | 0 | 2 | 2 | 2 | 2 | 2 | 2 | 2 | 2 |
| Lmo0493 | 2 | 2 | 2 | 2 | 2 | 2 | 2 | 2 | 2 | 2 | 2 |
| Lmo0497 | 2 | 2 | 2 | 2 | 0 | 0 | 0 | 0 | 0 | 0 | 0 |
| Lmo0525 | 0 | 0 | 0 | 0 | 0 | 0 | 0 | 0 | 0 | 0 | 0 |
| Lmo0549 | 2 | 2 | 2 | 2 | 2 | 2 | 2 | 2 | 2 | 2 | 2 |
| Lmo0630 | 2 | 2 | 2 | 2 | 2 | 2 | 2 | 2 | 2 | 2 | 2 |
| Lmo0631 | 2 | 2 | 2 | 2 | 2 | 2 | 2 | 2 | 2 | 2 | 2 |
| Lmo0632 | 2 | 2 | 2 | 2 | 2 | 2 | 2 | 2 | 2 | 2 | 2 |
| Lmo0732 | 2 | 2 | 0 | 2 | 2 | 2 | 2 | 2 | 2 | 0 | 2 |
| Lmo0733 | 2 | 2 | 2 | 2 | 2 | 2 | 2 | 2 | 2 | 2 | 2 |
| Lmo0734 | 0 | 0 | 0 | 0 | 0 | 0 | 0 | 0 | 0 | 0 | 0 |
| Lmo0735 | 0 | 0 | 0 | 0 | 0 | 0 | 0 | 0 | 0 | 0 | 0 |
| Lmo0739 | 0 | 0 | 0 | 0 | 0 | 0 | 0 | 0 | 0 | 0 | 0 |
| Lmo0750 | 0 | 0 | 0 | 0 | 2 | 0 | 0 | 0 | 0 | 0 | 0 |
| Lmo0751 | 2 | 2 | 2 | 2 | 2 | 2 | 2 | 2 | 2 | 2 | 2 |
| Lmo0752 | 2 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 | 2 |
| Lmo0780 | 0 | 2 | 0 | 2 | 2 | 2 | 0 | 0 | 0 | 0 | 0 |
| Lmo0801 | 0 | 0 | 0 | 0 | 2 | 2 | 2 | 0 | 0 | 0 | 0 |
| Lmo0804 | 0 | 2 | 2 | 0 | 2 | 2 | 2 | 0 | 0 | 0 | 0 |
| Lmo0832 | 2 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 | 2 |
| Lmo0842 | 0 | 2 | 2 | 2 | 2 | 2 | 2 | 0 | 2 | 0 | 0 |
| Lmo1030 | 2 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 | 2 |
| Lmo1060 | 0 | 0 | 0 | 0 | 0 | 0 | 0 | 0 | 0 | 0 | 0 |
| Lmo1061 | 0 | 0 | 0 | 0 | 0 | 0 | 0 | 0 | 0 | 0 | 0 |
| Lmo1063 | 0 | 0 | 0 | 0 | 0 | 0 | 0 | 0 | 0 | 0 | 0 |
| Lmo1076 | 2 | 2 | 2 | 2 | 0 | 0 | 0 | 0 | 0 | 0 | 0 |
| Lmo1077 | 2 | 2 | 2 | 2 | 0 | 0 | 0 | 0 | 0 | 0 | 0 |
| Lmo1080 | 2 | 2 | 2 | 2 | 0 | 0 | 0 | 0 | 0 | 0 | 0 |
| Lmo1081 | 2 | 2 | 2 | 2 | 0 | 0 | 0 | 0 | 0 | 0 | 0 |
| Lmo1082 | 2 | 2 | 2 | 2 | 0 | 0 | 0 | 0 | 0 | 0 | 0 |
| Lmo1085 | 2 | 2 | 2 | 2 | 0 | 0 | 0 | 0 | 0 | 0 | 0 |
| Lmo1088 | 2 | 2 | 2 | 2 | 0 | 0 | 0 | 0 | 0 | 0 | 0 |
| Lmo1090 | 2 | 2 | 2 | 2 | 0 | 0 | 0 | 0 | 0 | 0 | 0 |
| Lmo1091 | 2 | 2 | 2 | 2 | 0 | 0 | 0 | 0 | 0 | 0 | 0 |
| Lmo1102 | 2 | 2 | 0 | 0 | 2 | 2 | 2 | 2 | 2 | 2 | 2 |
| Lmo1115 | 0 | 0 | 0 | 0 | 0 | 0 | 0 | 0 | 0 | 0 | 0 |
| Lmo1116 | 2 | 2 | 2 | 2 | 2 | 2 | 2 | 0 | 2 | 2 | 2 |
| Lmo1118 | 0 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 | 0 |
| Lmo1120 | 2 | 2 | 2 | 2 | 2 | 2 | 2 | 2 | 2 | 2 | 2 |
| Lmo1133 | 2 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 | 2 |
| Lmo1307 | 0 | 0 | 0 | 0 | 0 | 0 | 0 | 0 | 0 | 0 | 0 |
| Lmo1441 | 2 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 | 2 |
| Lmo1597 | 2 | 2 | 2 | 0 | 0 | 0 | 0 | 0 | 0 | 0 | 0 |
| Lmo1604 | 0 | 2 | 0 | 2 | 2 | 2 | 2 | 2 | 2 | 2 | 2 |
| Lmo1648 | 0 | 0 | 0 | 0 | 2 | 0 | 0 | 0 | 0 | 0 | 0 |
| Lmo1666 | 2 | 2 | 2 | 2 | 2 | 2 | 2 | 2 | 2 | 2 | 2 |
| Lmo1714 | 2 | 2 | 2 | 2 | 2 | 2 | 2 | 2 | 2 | 2 | 2 |
| Lmo1968 | 0 | 0 | 0 | 0 | 0 | 0 | 0 | 0 | 0 | 0 | 0 |
| Lmo1973 | 0 | 0 | 0 | 0 | 0 | 0 | 0 | 0 | 0 | 0 | 0 |
| Lmo1974 | 0 | 0 | 0 | 0 | 0 | 0 | 0 | 0 | 0 | 0 | 0 |
| Lmo2026 | 0 | 0 | 0 | 0 | 0 | 0 | 0 | 0 | 0 | 0 | 0 |
| Lmo2027 | 0 | 2 | 0 | 0 | 2 | 2 | 2 | 2 | 2 | 0 | 0 |
| Lmo2276 | 2 | 2 | 0 | 0 | 2 | 2 | 2 | 2 | 2 | 2 | 0 |
| Lmo2277 | 2 | 2 | 0 | 0 | 2 | 0 | 2 | 0 | 0 | 0 | 0 |
| Lmo2311 | 0 | 0 | 0 | 0 | 0 | 0 | 0 | 0 | 0 | 0 | 0 |
| Lmo2312 | 2 | 2 | 0 | 2 | 0 | 0 | 2 | 2 | 0 | 0 | 2 |
| Lmo2324 | 0 | 0 | 0 | 2 | 0 | 0 | 2 | 2 | 0 | 2 | 2 |
| Lmo2408 | 0 | 0 | 0 | 0 | 0 | 0 | 0 | 0 | 0 | 0 | 0 |
| Lmo2443 | 2 | 2 | 2 | 2 | 2 | 2 | 2 | 2 | 2 | 2 | 2 |
| Lmo2576 | 0 | 0 | 0 | 0 | 0 | 0 | 0 | 0 | 0 | 0 | 0 |
| Lmo2594 | 2 | 2 | 2 | 2 | 2 | 2 | 2 | 2 | 2 | 2 | 2 |
| Lmo2595 | 0 | 0 | 0 | 2 | 2 | 2 | 2 | 2 | 2 | 2 | 2 |
| Lmo2671 | 2 | 2 | 2 | 2 | 2 | 2 | 2 | 2 | 2 | 2 | 2 |
| Lmo2686 | 0 | 0 | 0 | 0 | 0 | 0 | 0 | 0 | 0 | 0 | 0 |
| Lmo2732 | 2 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 | 2 |
| Lmo2786 | 0 | 0 | 0 | 0 | 0 | 0 | 0 | 0 | 0 | 0 | 0 |
| Lmo2787 | 0 | 0 | 0 | 0 | 0 | 0 | 0 | 0 | 0 | 0 | 0 |
| Lmo2807 | 2 | 0 | 0 | 2 | 2 | 2 | 2 | 0 | 2 | 0 | 0 |
| Lmo2809 | 2 | 2 | 2 | 2 | 0 | 0 | 0 | 2 | 0 | 0 | 0 |
| Lmo2821 | 2 | 2 | 2 | 2 | 2 | 2 | 2 | 2 | 2 | 2 | 2 |
| Lin0060 | 0 | 0 | 0 | 0 | 0 | 0 | 0 | 2 | 0 | 0 | 0 |
| Lin0197 | 0 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 | 0 |
| Lin0415 | 0 | 2 | 0 | 0 | 2 | 2 | 2 | 2 | 2 | 2 | 2 |
| Lin0453 | 0 | 0 | 0 | 0 | 2 | 2 | 2 | 2 | 2 | 2 | 2 |
| Lin0454 | 0 | 0 | 0 | 0 | 2 | 2 | 2 | 2 | 2 | 2 | 2 |
| Lin0455 | 0 | 0 | 0 | 0 | 2 | 2 | 2 | 0 | 2 | 0 | 2 |
| Lin0465 | 0 | 0 | 0 | 0 | 0 | 0 | 0 | 0 | 0 | 0 | 0 |
| Lin0476 | 0 | 2 | 0 | 0 | 2 | 2 | 0 | 0 | 2 | 0 | 0 |
| Lin0661 | 0 | 0 | 0 | 0 | 2 | 2 | 0 | 0 | 2 | 0 | 0 |
| Lin0665 | 2 | 2 | 2 | 2 | 2 | 2 | 2 | 2 | 2 | 2 | 2 |
| Lin0677 | 0 | 0 | 0 | 0 | 0 | 0 | 0 | 0 | 0 | 0 | 0 |
| Lin0739 | 0 | 0 | 0 | 0 | 0 | 0 | 0 | 0 | 0 | 0 | 0 |
| Lin0740 | 0 | 0 | 0 | 0 | 2 | 2 | 2 | 0 | 2 | 0 | 0 |
| Lin0803 | 0 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 | 0 |
| Lin1066 | 0 | 0 | 0 | 0 | 0 | 0 | 0 | 0 | 0 | 0 | 0 |
| Lin1069 | 0 | 0 | 0 | 0 | 0 | 0 | 0 | 0 | 0 | 0 | 0 |
| Lin1073 | 0 | 0 | 0 | 0 | 2 | 2 | 2 | 2 | 2 | 2 | 2 |
| Lin1083 | 0 | 0 | 0 | 0 | 0 | 0 | 0 | 0 | 0 | 0 | 0 |
| Lin1084 | 2 | 2 | 2 | 2 | 2 | 2 | 2 | 2 | 2 | 2 | 2 |
| Lin1090 | 2 | 2 | 2 | 2 | 2 | 2 | 2 | 2 | 2 | 2 | 2 |
| Lin1204 | 2 | 2 | 2 | 2 | 2 | 2 | 2 | 2 | 2 | 2 | 2 |
| Lin1262 | 0 | 2 | 0 | 0 | 2 | 2 | 2 | 2 | 2 | 2 | 2 |
| Lin1299 | 2 | 0 | 0 | 0 | 2 | 2 | 2 | 0 | 2 | 2 | 2 |
| Lin1328 | 0 | 2 | 0 | 0 | 2 | 2 | 2 | 2 | 2 | 2 | 2 |
| Lin1452 | 0 | 0 | 0 | 2 | 0 | 0 | 0 | 0 | 0 | 0 | 0 |
| Lin1621 | 0 | 0 | 0 | 0 | 0 | 0 | 0 | 0 | 0 | 0 | 0 |
| Lin2397 | 0 | 0 | 0 | 2 | 0 | 0 | 2 | 0 | 0 | 0 | 0 |
| Lin2494 | 2 | 2 | 2 | 0 | 0 | 2 | 2 | 0 | 0 | 0 | 0 |
| Lin2603 | 2 | 2 | 0 | 0 | 0 | 0 | 0 | 0 | 0 | 0 | 2 |
| Lin2724 | 0 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 | 0 |
| Lin2743 | 2 | 2 | 2 | 0 | 0 | 0 | 0 | 0 | 0 | 0 | 0 |
| Lin2744 | 2 | 2 | 2 | 0 | 0 | 0 | 0 | 2 | 0 | 0 | 0 |
| Lin2839 | 0 | 0 | 0 | 0 | 0 | 0 | 0 | 0 | 0 | 0 | 0 |
| Lin2918 | 0 | 0 | 0 | 0 | 0 | 0 | 0 | 0 | 0 | 0 | 0 |
| ORF 105 | 2 | 2 | 2 | 0 | 2 | 2 | 2 | 0 | 2 | 2 | 2 |
| ORF 1390 | 2 | 0 | 0 | 0 | 2 | 2 | 2 | 0 | 0 | 2 | 0 |
| ORF 1445 | 0 | 0 | 0 | 0 | 2 | 2 | 2 | 2 | 2 | 2 | 2 |
| ORF 1490 | 2 | 2 | 2 | 2 | 2 | 2 | 2 | 2 | 2 | 2 | 2 |
| ORF 1491 | 2 | 2 | 2 | 2 | 2 | 2 | 2 | 2 | 2 | 2 | 2 |
| ORF 1590 | 2 | 2 | 2 | 2 | 2 | 2 | 2 | 2 | 2 | 2 | 2 |
| ORF 1761 | 2 | 2 | 2 | 2 | 2 | 2 | 2 | 2 | 2 | 2 | 2 |
| ORF 2017 | 2 | 2 | 2 | 2 | 2 | 2 | 2 | 2 | 2 | 2 | 2 |
| ORF 2074 | 2 | 2 | 2 | 2 | 2 | 2 | 2 | 2 | 2 | 2 | 2 |
| ORF 2076 | 2 | 2 | 2 | 2 | 2 | 2 | 2 | 2 | 2 | 2 | 2 |
| ORF 2110 | 0 | 0 | 0 | 0 | 2 | 2 | 2 | 2 | 2 | 2 | 2 |
| ORF 2372 | 0 | 0 | 0 | 0 | 2 | 2 | 2 | 2 | 2 | 2 | 2 |
| ORF 2541 | 2 | 2 | 2 | 2 | 2 | 2 | 2 | 2 | 2 | 2 | 2 |
| ORF 2579 | 2 | 2 | 2 | 2 | 2 | 2 | 2 | 2 | 2 | 2 | 2 |
| ORF 2777 | 2 | 2 | 2 | 2 | 2 | 2 | 2 | 2 | 2 | 2 | 2 |
| ORF 2819 | 2 | 2 | 2 | 2 | 2 | 2 | 2 | 2 | 2 | 2 | 2 |
| ORF 2847 | 0 | 0 | 0 | 0 | 2 | 2 | 2 | 0 | 2 | 2 | 2 |
| ORF 2858 | 2 | 2 | 2 | 2 | 2 | 2 | 2 | 2 | 2 | 2 | 2 |
| ORF 3103 | 2 | 2 | 2 | 0 | 0 | 0 | 0 | 2 | 0 | 0 | 0 |
| ORF 3184 | 2 | 2 | 2 | 2 | 2 | 2 | 2 | 2 | 2 | 2 | 2 |
| ORF 3401 | 2 | 2 | 0 | 2 | 2 | 2 | 2 | 2 | 2 | 2 | 2 |
| ORF 3792 | 2 | 2 | 2 | 2 | 2 | 2 | 2 | 2 | 2 | 2 | 2 |
| ORF 3840 | 2 | 2 | 2 | 2 | 2 | 2 | 2 | 2 | 2 | 2 | 2 |
| ORF 4101 | 2 | 2 | 2 | 2 | 2 | 2 | 2 | 2 | 2 | 2 | 2 |
| ORF 660 | 0 | 0 | 0 | 2 | 2 | 2 | 2 | 2 | 2 | 2 | 2 |
| ORF 757 | 0 | 0 | 0 | 2 | 2 | 2 | 2 | 2 | 2 | 2 | 2 |
| ORF 799 | 0 | 0 | 0 | 0 | 2 | 2 | 2 | 2 | 2 | 2 | 2 |
| ORF 817 | 2 | 2 | 2 | 2 | 0 | 0 | 0 | 2 | 0 | 0 | 0 |

| | | CLIP42639- | CLIP42652- | CLIP80459- | | | | | | | |
|---|---|---|---|---|---|---|---|---|---|---|---|
| Gene | CLIP8715-4b | 4b Epi | 4b Epi | 4b Epi | CLIP87889-4b | CLIP87896-4b | CLIP87911-4b | CLIP87915-4b | CLIP87917-4b | CLIP74910-4b | CLIP60383-4b |
| Lmo0017 | 2 | 2 | 2 | 2 | 2 | 2 | 2 | 2 | 2 | 2 | 2 |
| Lmo0036 | 2 | 2 | 2 | 2 | 2 | 2 | 2 | 2 | 2 | 2 | 2 |
| Lmo0037 | 2 | 2 | 2 | 2 | 2 | 2 | 2 | 2 | 2 | 2 | 2 |
| Lmo0066 | 0 | 0 | 2 | 0 | 0 | 0 | 0 | 0 | 0 | 0 | 0 |
| Lmo0067 | 0 | 0 | 0 | 0 | 0 | 0 | 0 | 0 | 0 | 0 | 0 |
| Lmo0068 | 2 | 2 | 2 | 0 | 0 | 0 | 2 | 2 | 2 | 0 | 0 |
| Lmo0069 | 2 | 2 | 2 | 0 | 0 | 0 | 2 | 2 | 2 | 0 | 0 |
| Lmo0071 | 2 | 2 | 2 | 0 | 0 | 0 | 2 | 2 | 2 | 0 | 0 |
| Lmo0072 | 2 | 2 | 2 | 2 | 2 | 2 | 2 | 2 | 2 | 2 | 2 |
| Lmo0079 | 2 | 2 | 2 | 0 | 0 | 2 | 0 | 2 | 2 | 2 | 0 |
| Lmo0080 | 2 | 2 | 2 | 0 | 0 | 2 | 2 | 2 | 2 | 2 | 0 |
| Lmo0081 | 0 | 0 | 0 | 0 | 0 | 0 | 0 | 0 | 0 | 0 | 0 |
| Lmo0082 | 2 | 2 | 2 | 2 | 2 | 2 | 2 | 2 | 2 | 2 | 2 |
| Lmo0083 | 2 | 2 | 2 | 2 | 2 | 2 | 2 | 2 | 2 | 2 | 2 |
| Lmo0094 | 2 | 2 | 2 | 2 | 2 | 2 | 2 | 2 | 2 | 2 | 2 |
| Lmo0106 | 2 | 2 | 2 | 2 | 2 | 0 | 2 | 2 | 2 | 2 | 2 |
| Lmo0141 | 2 | 2 | 2 | 2 | 2 | 2 | 2 | 2 | 2 | 2 | 0 |
| Lmo0143 | 0 | 0 | 0 | 0 | 2 | 0 | 0 | 0 | 0 | 0 | 0 |
| Lmo0144 | 2 | 2 | 2 | 2 | 2 | 2 | 2 | 2 | 2 | 2 | 0 |
| Lmo0147 | 2 | 2 | 2 | 0 | 0 | 0 | 2 | 2 | 2 | 2 | 2 |
| Lmo0151 | 0 | 0 | 0 | 0 | 0 | 0 | 0 | 0 | 0 | 0 | 0 |
| Lmo0171 | 0 | 0 | 0 | 0 | 0 | 0 | 0 | 0 | 0 | 0 | 0 |
| Lmo0172 | 0 | 0 | 0 | 0 | 0 | 0 | 0 | 0 | 0 | 0 | 0 |
| Lmo0252 | 0 | 0 | 0 | 0 | 0 | 0 | 0 | 0 | 0 | 0 | 0 |
| Lmo0253 | 0 | 0 | 0 | 0 | 0 | 0 | 0 | 0 | 0 | 0 | 0 |
| Lmo0304 | 0 | 0 | 0 | 0 | 0 | 2 | 0 | 0 | 0 | 0 | 0 |
| Lmo0310 | 0 | 0 | 0 | 0 | 0 | 0 | 0 | 0 | 0 | 0 | 0 |
| Lmo0320 | 2 | 2 | 2 | 2 | 2 | 2 | 2 | 2 | 2 | 2 | 2 |
| Lmo0333 | 2 | 2 | 2 | 2 | 2 | 2 | 2 | 2 | 2 | 2 | 2 |
| Lmo0334 | 2 | 2 | 2 | 2 | 2 | 2 | 2 | 2 | 2 | 2 | 2 |
| Lmo0338 | 0 | 0 | 0 | 0 | 0 | 0 | 0 | 0 | 0 | 0 | 0 |
| Lmo0378 | 0 | 0 | 0 | 2 | 2 | 0 | 0 | 0 | 0 | 0 | 2 |
| Lmo0379 | 0 | 0 | 0 | 0 | 0 | 0 | 0 | 0 | 0 | 0 | 0 |
| Lmo0380 | 0 | 0 | 0 | 2 | 2 | 0 | 0 | 0 | 0 | 0 | 2 |
| Lmo0381 | 0 | 0 | 0 | 2 | 2 | 0 | 0 | 0 | 0 | 0 | 2 |
| Lmo0409 | 0 | 2 | 2 | 0 | 0 | 2 | 2 | 2 | 2 | 2 | 2 |
| Lmo0419 | 2 | 2 | 2 | 2 | 2 | 2 | 2 | 2 | 2 | 2 | 2 |
| Lmo0440 | 2 | 2 | 2 | 2 | 2 | 2 | 2 | 2 | 2 | 2 | 2 |
| Lmo0444 | 0 | 0 | 0 | 0 | 0 | 0 | 0 | 0 | 0 | 0 | 0 |
| Lmo0448 | 0 | 2 | 2 | 2 | 0 | 2 | 0 | 0 | 0 | 0 | 2 |
| Lmo0459 | 2 | 2 | 2 | 2 | 2 | 2 | 2 | 2 | 2 | 2 | 2 |
| Lmo0462 | 2 | 2 | 2 | 2 | 2 | 2 | 2 | 2 | 2 | 2 | 2 |
| Lmo0466 | 0 | 0 | 0 | 0 | 0 | 0 | 0 | 0 | 0 | 0 | 0 |
| Lmo0467 | 0 | 0 | 0 | 0 | 0 | 0 | 0 | 0 | 0 | 0 | 0 |
| Lmo0471 | 0 | 0 | 0 | 0 | 0 | 0 | 0 | 0 | 0 | 0 | 0 |
| Lmo0472 | 0 | 0 | 0 | 0 | 0 | 0 | 0 | 0 | 2 | 0 | 0 |
| Lmo0473 | 0 | 2 | 2 | 2 | 2 | 0 | 2 | 2 | 2 | 0 | 2 |
| Lmo0474 | 2 | 2 | 2 | 2 | 2 | 2 | 2 | 2 | 2 | 2 | 2 |
| Lmo0493 | 2 | 2 | 2 | 2 | 2 | 2 | 2 | 2 | 2 | 2 | 2 |
| Lmo0497 | 0 | 0 | 0 | 0 | 0 | 0 | 0 | 0 | 0 | 0 | 0 |
| Lmo0525 | 0 | 0 | 0 | 0 | 0 | 0 | 0 | 0 | 0 | 0 | 0 |
| Lmo0549 | 2 | 2 | 2 | 2 | 2 | 2 | 2 | 2 | 2 | 2 | 2 |
| Lmo0630 | 2 | 2 | 2 | 0 | 0 | 2 | 2 | 2 | 2 | 2 | 0 |
| Lmo0631 | 2 | 2 | 2 | 0 | 0 | 2 | 2 | 2 | 2 | 2 | 0 |
| Lmo0632 | 2 | 2 | 2 | 0 | 0 | 2 | 2 | 2 | 2 | 2 | 0 |
| Lmo0732 | 0 | 2 | 2 | 2 | 0 | 0 | 0 | 2 | 2 | 0 | 2 |
| Lmo0733 | 2 | 2 | 2 | 2 | 2 | 2 | 2 | 2 | 2 | 2 | 2 |
| Lmo0734 | 0 | 0 | 0 | 0 | 0 | 0 | 0 | 0 | 0 | 0 | 0 |
| Lmo0735 | 0 | 0 | 0 | 0 | 0 | 0 | 0 | 0 | 0 | 0 | 0 |
| Lmo0739 | 0 | 0 | 0 | 0 | 0 | 0 | 0 | 0 | 0 | 0 | 0 |
| Lmo0750 | 0 | 0 | 0 | 0 | 0 | 0 | 0 | 0 | 0 | 0 | 0 |
| Lmo0751 | 2 | 2 | 2 | 0 | 0 | 0 | 2 | 2 | 2 | 2 | 0 |
| Lmo0752 | 2 | 2 | 2 | 2 | 2 | 2 | 2 | 2 | 2 | 2 | 2 |
| Lmo0753 | 2 | 2 | 2 | 2 | 2 | 2 | 2 | 2 | 2 | 2 | 2 |
| Lmo0780 | 0 | 0 | 0 | 0 | 0 | 0 | 0 | 0 | 0 | 0 | 0 |
| Lmo0801 | 0 | 2 | 2 | 0 | 0 | 0 | 0 | 0 | 0 | 0 | 0 |
| Lmo0804 | 0 | 0 | 0 | 0 | 0 | 2 | 2 | 2 | 0 | 2 | 0 |
| Lmo0832 | 2 | 2 | 2 | 2 | 2 | 2 | 2 | 2 | 2 | 2 | 2 |
| Lmo0833 | 2 | 2 | 2 | 2 | 2 | 2 | 2 | 2 | 2 | 2 | 2 |
| Lmo0834 | 2 | 2 | 2 | 2 | 2 | 2 | 2 | 2 | 2 | 2 | 2 |
| Lmo0835 | 2 | 2 | 2 | 2 | 2 | 2 | 2 | 2 | 2 | 2 | 2 |
| Lmo0842 | 0 | 2 | 2 | 0 | 0 | 0 | 0 | 0 | 0 | 0 | 2 |
| Lmo1030 | 2 | 2 | 2 | 2 | 2 | 2 | 2 | 2 | 2 | 2 | 2 |
| Lmo1036 | 2 | 2 | 2 | 2 | 2 | 2 | 2 | 2 | 2 | 2 | 2 |
| Lmo1060 | 0 | 0 | 0 | 0 | 0 | 0 | 0 | 0 | 0 | 0 | 0 |
| Lmo1061 | 0 | 0 | 0 | 0 | 0 | 0 | 0 | 0 | 0 | 0 | 0 |
| Lmo1063 | 0 | 0 | 0 | 0 | 0 | 0 | 0 | 0 | 0 | 0 | 0 |
| Lmo1076 | 0 | 0 | 0 | 0 | 0 | 0 | 0 | 0 | 0 | 0 | 0 |
| Lmo1077 | 0 | 0 | 0 | 0 | 0 | 0 | 0 | 0 | 0 | 0 | 0 |
| Lmo1080 | 0 | 0 | 0 | 0 | 0 | 0 | 0 | 0 | 0 | 0 | 0 |
| Lmo1081 | 0 | 0 | 0 | 0 | 0 | 0 | 0 | 0 | 0 | 0 | 0 |
| Lmo1082 | 0 | 0 | 0 | 0 | 0 | 0 | 0 | 0 | 0 | 0 | 0 |
| Lmo1085 | 0 | 0 | 0 | 0 | 0 | 0 | 0 | 0 | 0 | 0 | 0 |
| Lmo1088 | 0 | 0 | 0 | 0 | 0 | 0 | 0 | 0 | 0 | 0 | 0 |
| Lmo1090 | 0 | 0 | 0 | 0 | 0 | 0 | 0 | 0 | 0 | 0 | 0 |
| Lmo1091 | 0 | 0 | 0 | 0 | 0 | 0 | 0 | 0 | 0 | 0 | 0 |
| Lmo1102 | 2 | 2 | 2 | 2 | 0 | 0 | 0 | 2 | 2 | 0 | 2 |
| Lmo1115 | 0 | 0 | 0 | 0 | 0 | 0 | 0 | 0 | 0 | 0 | 0 |
| Lmo1116 | 2 | 2 | 2 | 2 | 2 | 2 | 2 | 2 | 2 | 0 | 2 |
| Lmo1118 | 0 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 | 0 |
| Lmo1120 | 2 | 2 | 2 | 0 | 0 | 2 | 2 | 2 | 2 | 2 | 0 |
| Lmo1133 | 2 | 2 | 2 | 2 | 2 | 2 | 2 | 2 | 2 | 2 | 2 |
| Lmo1135 | 2 | 2 | 2 | 2 | 2 | 2 | 2 | 2 | 2 | 2 | 2 |
| Lmo1307 | 0 | 0 | 0 | 0 | 0 | 0 | 0 | 0 | 0 | 0 | 0 |
| Lmo1441 | 2 | 2 | 2 | 2 | 2 | 2 | 2 | 2 | 2 | 2 | 2 |
| Lmo1451 | 2 | 2 | 2 | 2 | 2 | 2 | 2 | 2 | 2 | 2 | 2 |
| Lmo1597 | 0 | 0 | 0 | 2 | 2 | 2 | 0 | 0 | 0 | 0 | 2 |
| Lmo1604 | 2 | 2 | 2 | 2 | 0 | 0 | 0 | 0 | 0 | 0 | 0 |
| Lmo1648 | 0 | 0 | 0 | 0 | 0 | 0 | 0 | 0 | 0 | 0 | 0 |
| Lmo1666 | 2 | 2 | 2 | 2 | 2 | 2 | 2 | 2 | 2 | 2 | 2 |
| Lmo1714 | 2 | 2 | 2 | 2 | 2 | 2 | 2 | 2 | 2 | 2 | 2 |
| Lmo1968 | 0 | 0 | 0 | 0 | 0 | 0 | 0 | 0 | 0 | 0 | 0 |
| Lmo1973 | 0 | 0 | 0 | 0 | 0 | 0 | 0 | 0 | 0 | 0 | 0 |
| Lmo1974 | 0 | 0 | 0 | 0 | 0 | 0 | 0 | 0 | 0 | 0 | 0 |
| Lmo2026 | 0 | 0 | 0 | 0 | 0 | 0 | 0 | 0 | 0 | 0 | 0 |
| Lmo2027 | 0 | 2 | 2 | 2 | 2 | 0 | 0 | 2 | 0 | 0 | 2 |
| Lmo2276 | 2 | 2 | 2 | 2 | 0 | 0 | 0 | 0 | 0 | 0 | 2 |
| Lmo2277 | 0 | 2 | 0 | 0 | 0 | 0 | 0 | 0 | 0 | 0 | 0 |
| Lmo2311 | 0 | 0 | 0 | 0 | 0 | 0 | 0 | 0 | 0 | 0 | 0 |
| Lmo2312 | 0 | 0 | 0 | 0 | 0 | 2 | 2 | 2 | 0 | 0 | 0 |
| Lmo2324 | 2 | 0 | 0 | 0 | 0 | 2 | 2 | 2 | 0 | 0 | 0 |
| Lmo2408 | 0 | 0 | 0 | 0 | 0 | 0 | 0 | 0 | 0 | 0 | 0 |
| Lmo2443 | 2 | 2 | 2 | 2 | 2 | 2 | 2 | 2 | 2 | 2 | 2 |
| Lmo2576 | 0 | 0 | 0 | 0 | 0 | 0 | 0 | 0 | 0 | 0 | 0 |
| Lmo2594 | 2 | 2 | 2 | 2 | 2 | 2 | 2 | 2 | 2 | 2 | 2 |
| Lmo2595 | 2 | 2 | 2 | 2 | 2 | 2 | 2 | 2 | 2 | 2 | 2 |
| Lmo2671 | 2 | 2 | 2 | 2 | 2 | 2 | 2 | 2 | 2 | 2 | 2 |
| Lmo2686 | 0 | 0 | 0 | 0 | 0 | 0 | 0 | 0 | 0 | 0 | 0 |
| Lmo2732 | 2 | 2 | 2 | 2 | 2 | 2 | 2 | 2 | 2 | 2 | 2 |
| Lmo2736 | 2 | 2 | 2 | 2 | 2 | 2 | 2 | 2 | 2 | 2 | 2 |
| Lmo2786 | 0 | 0 | 0 | 0 | 0 | 0 | 0 | 0 | 0 | 0 | 0 |
| Lmo2787 | 0 | 0 | 0 | 0 | 0 | 0 | 0 | 0 | 0 | 0 | 0 |
| Lmo2807 | 0 | 2 | 2 | 0 | 0 | 0 | 2 | 2 | 2 | 0 | 0 |
| Lmo2809 | 0 | 0 | 0 | 2 | 2 | 2 | 0 | 0 | 0 | 2 | 2 |
| Lmo2821 | 2 | 2 | 2 | 2 | 2 | 2 | 2 | 2 | 2 | 2 | 2 |
| Lin0060 | 0 | 0 | 0 | 0 | 0 | 0 | 0 | 0 | 0 | 2 | 0 |
| Lin0197 | 0 | 0 | 0 | 0 | 0 | 0 | 0 | 0 | 0 | 0 | 0 |
| Lin0332 | 0 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 | 0 |
| Lin0415 | 2 | 2 | 2 | 2 | 2 | 2 | 2 | 0 | 0 | 0 | 2 |
| Lin0453 | 2 | 2 | 2 | 2 | 2 | 2 | 2 | 2 | 2 | 2 | 2 |
| Lin0454 | 2 | 2 | 2 | 2 | 2 | 2 | 2 | 2 | 2 | 2 | 2 |
| Lin0455 | 0 | 2 | 2 | 2 | 0 | 2 | 2 | 2 | 2 | 2 | 0 |
| Lin0465 | 0 | 0 | 0 | 0 | 0 | 0 | 0 | 0 | 0 | 0 | 0 |
| Lin0476 | 0 | 2 | 2 | 0 | 0 | 0 | 0 | 0 | 0 | 0 | 0 |
| Lin0661 | 0 | 2 | 0 | 0 | 0 | 0 | 0 | 0 | 0 | 0 | 0 |
| Lin0665 | 2 | 2 | 2 | 2 | 2 | 2 | 2 | 2 | 2 | 2 | 2 |
| Lin0677 | 0 | 0 | 0 | 0 | 0 | 0 | 0 | 0 | 0 | 0 | 0 |
| Lin0739 | 0 | 0 | 0 | 0 | 0 | 0 | 0 | 0 | 0 | 0 | 0 |
| Lin0740 | 0 | 2 | 2 | 0 | 0 | 0 | 0 | 0 | 0 | 0 | 0 |
| Lin0803 | 0 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 | 0 |
| Lin1066 | 0 | 0 | 0 | 0 | 0 | 2 | 0 | 0 | 0 | 0 | 0 |
| Lin1069 | 0 | 0 | 0 | 0 | 0 | 0 | 0 | 0 | 0 | 0 | 0 |
| Lin1073 | 2 | 2 | 2 | 2 | 2 | 2 | 2 | 2 | 2 | 2 | 2 |
| Lin1083 | 0 | 0 | 0 | 0 | 0 | 0 | 0 | 0 | 0 | 0 | 0 |
| Lin1084 | 2 | 2 | 0 | 0 | 2 | 2 | 2 | 2 | 2 | 2 | 0 |
| Lin1090 | 2 | 2 | 2 | 2 | 2 | 2 | 2 | 2 | 2 | 2 | 2 |
| Lin1204 | 2 | 2 | 2 | 2 | 2 | 2 | 2 | 2 | 2 | 2 | 2 |
| Lin1262 | 2 | 2 | 2 | 2 | 0 | 0 | 0 | 0 | 0 | 0 | 2 |
| Lin1299 | 2 | 2 | 2 | 2 | 2 | 0 | 2 | 2 | 2 | 0 | 2 |
| Lin1328 | 2 | 2 | 2 | 2 | 0 | 0 | 0 | 0 | 0 | 0 | 2 |
| Lin1452 | 0 | 0 | 0 | 0 | 0 | 0 | 0 | 0 | 0 | 0 | 0 |
| Lin1621 | 0 | 0 | 0 | 0 | 0 | 0 | 0 | 0 | 0 | 0 | 0 |
| Lin2397 | 0 | 0 | 0 | 0 | 0 | 2 | 0 | 0 | 0 | 0 | 0 |
| Lin2494 | 0 | 2 | 0 | 2 | 2 | 2 | 0 | 0 | 0 | 0 | 2 |
| Lin2603 | 0 | 0 | 0 | 0 | 0 | 2 | 2 | 0 | 0 | 0 | 0 |
| Lin2724 | 0 | 0 | 0 | 0 | 0 | 0 | 0 | 0 | 0 | 0 | 0 |
| Lin2741 | 0 | 0 | 0 | 0 | 0 | 0 | 0 | 0 | 0 | 0 | 0 |
| Lin2743 | 0 | 0 | 0 | 0 | 0 | 0 | 0 | 0 | 0 | 0 | 0 |
| Lin2744 | 0 | 0 | 0 | 2 | 0 | 0 | 0 | 0 | 0 | 0 | 0 |
| Lin2839 | 0 | 0 | 0 | 0 | 0 | 0 | 0 | 0 | 0 | 0 | 0 |
| Lin2918 | 0 | 0 | 0 | 0 | 0 | 0 | 0 | 0 | 0 | 0 | 0 |
| ORF 105 | 2 | 2 | 2 | 2 | 2 | 0 | 2 | 2 | 2 | 0 | 2 |
| ORF 1390 | 2 | 2 | 2 | 2 | 2 | 0 | 0 | 0 | 2 | 0 | 2 |
| ORF 1445 | 2 | 2 | 2 | 2 | 2 | 2 | 2 | 2 | 2 | 2 | 2 |
| ORF 1490 | 2 | 2 | 2 | 2 | 2 | 2 | 2 | 2 | 2 | 2 | 2 |
| ORF 1491 | 2 | 2 | 2 | 2 | 2 | 2 | 2 | 2 | 2 | 2 | 2 |
| ORF 1590 | 2 | 2 | 2 | 2 | 2 | 2 | 2 | 2 | 2 | 2 | 2 |
| ORF 1761 | 2 | 2 | 2 | 2 | 2 | 2 | 2 | 2 | 2 | 2 | 2 |
| ORF 2017 | 2 | 2 | 2 | 2 | 2 | 2 | 2 | 2 | 2 | 2 | 2 |
| ORF 2074 | 2 | 2 | 2 | 2 | 2 | 2 | 2 | 2 | 2 | 2 | 2 |
| ORF 2076 | 2 | 2 | 2 | 2 | 2 | 2 | 2 | 2 | 2 | 2 | 2 |
| ORF 2110 | 2 | 2 | 2 | 2 | 2 | 2 | 2 | 2 | 2 | 2 | 2 |
| ORF 2372 | 2 | 2 | 2 | 2 | 2 | 2 | 2 | 2 | 2 | 2 | 2 |
| ORF 2541 | 2 | 2 | 2 | 2 | 2 | 2 | 2 | 2 | 2 | 2 | 2 |
| ORF 2579 | 2 | 2 | 2 | 2 | 2 | 2 | 2 | 2 | 2 | 2 | 2 |
| ORF 2777 | 2 | 2 | 2 | 2 | 2 | 2 | 2 | 2 | 2 | 2 | 2 |
| ORF 2819 | 2 | 2 | 2 | 2 | 2 | 2 | 2 | 2 | 2 | 2 | 2 |
| ORF 2847 | 2 | 2 | 2 | 2 | 2 | 0 | 2 | 2 | 2 | 0 | 2 |
| ORF 2858 | 2 | 2 | 2 | 2 | 2 | 2 | 2 | 2 | 2 | 2 | 2 |
| ORF 3103 | 0 | 0 | 0 | 2 | 2 | 2 | 0 | 0 | 0 | 2 | 2 |
| ORF 3184 | 2 | 2 | 2 | 2 | 2 | 2 | 2 | 2 | 2 | 2 | 2 |
| ORF 3401 | 2 | 2 | 2 | 2 | 2 | 2 | 2 | 2 | 2 | 2 | 2 |
| ORF 3792 | 2 | 2 | 2 | 2 | 2 | 2 | 2 | 2 | 2 | 2 | 2 |
| ORF 3840 | 2 | 2 | 2 | 2 | 2 | 2 | 2 | 2 | 2 | 2 | 2 |
| ORF 4101 | 2 | 2 | 2 | 2 | 2 | 2 | 2 | 2 | 2 | 2 | 2 |
| ORF 660 | 2 | 2 | 2 | 2 | 2 | 2 | 2 | 2 | 2 | 2 | 2 |
| ORF 757 | 2 | 2 | 2 | 2 | 2 | 2 | 2 | 2 | 2 | 2 | 2 |
| ORF 799 | 2 | 2 | 2 | 2 | 2 | 2 | 2 | 2 | 2 | 2 | 2 |
| ORF 817 | 0 | 0 | 0 | 2 | 2 | 2 | 0 | 0 | 0 | 2 | 2 |

| Gene | CLIP72783-4b | CLIP22573-4b Epi | CLIP73722-4b | CLIP87691-4b | CLIP79619-4d | CLIP 74912-4d |
|---|---|---|---|---|---|---|
| Lmo0017 | 2 | 2 | 2 | 2 | 2 | 2 |
| Lmo0036 | 2 | 2 | 2 | 2 | 2 | 2 |
| Lmo0037 | 2 | 2 | 2 | 2 | 2 | 2 |
| Lmo0066 | 0 | 0 | 0 | 0 | 0 | 0 |
| Lmo0067 | 0 | 0 | 0 | 0 | 0 | 0 |
| Lmo0068 | 2 | 2 | 0 | 0 | 0 | 0 |
| Lmo0069 | 2 | 2 | 0 | 0 | 0 | 0 |
| Lmo0071 | 2 | 2 | 0 | 0 | 0 | 0 |
| Lmo0072 | 2 | 2 | 2 | 2 | 2 | 2 |
| Lmo0079 | 2 | 2 | 2 | 2 | 0 | 2 |
| Lmo0080 | 2 | 2 | 2 | 2 | 0 | 2 |
| Lmo0081 | 0 | 0 | 0 | 0 | 0 | 0 |
| Lmo0082 | 2 | 2 | 2 | 2 | 2 | 2 |
| Lmo0083 | 2 | 2 | 2 | 2 | 2 | 2 |
| Lmo0094 | 2 | 2 | 2 | 2 | 2 | 2 |
| Lmo0106 | 2 | 2 | 2 | 2 | 2 | 2 |
| Lmo0141 | 2 | 2 | 2 | 2 | 2 | 2 |
| Lmo0143 | 0 | 0 | 0 | 0 | 2 | 0 |
| Lmo0144 | 2 | 2 | 2 | 2 | 2 | 2 |
| Lmo0147 | 2 | 2 | 0 | 2 | 2 | 2 |
| Lmo0151 | 0 | 0 | 0 | 0 | 0 | 0 |
| Lmo0171 | 0 | 0 | 0 | 0 | 0 | 0 |
| Lmo0172 | 0 | 0 | 0 | 0 | 0 | 0 |
| Lmo0252 | 0 | 0 | 0 | 0 | 0 | 0 |
| Lmo0253 | 0 | 0 | 0 | 0 | 0 | 0 |
| Lmo0304 | 0 | 0 | 2 | 0 | 0 | 0 |
| Lmo0310 | 0 | 0 | 2 | 0 | 0 | 0 |
| Lmo0320 | 2 | 2 | 2 | 2 | 2 | 2 |
| Lmo0333 | 2 | 2 | 2 | 2 | 2 | 2 |
| Lmo0334 | 2 | 2 | 2 | 2 | 2 | 2 |
| Lmo0338 | 0 | 0 | 0 | 0 | 0 | 0 |
| Lmo0378 | 0 | 0 | 2 | 0 | 2 | 0 |
| Lmo0379 | 0 | 0 | 0 | 0 | 0 | 0 |
| Lmo0380 | 0 | 0 | 2 | 0 | 2 | 0 |
| Lmo0381 | 0 | 0 | 2 | 0 | 2 | 0 |
| Lmo0409 | 2 | 2 | 2 | 2 | 2 | 2 |
| Lmo0419 | 2 | 2 | 2 | 2 | 2 | 2 |
| Lmo0440 | 2 | 2 | 2 | 2 | 2 | 2 |
| Lmo0444 | 0 | 0 | 0 | 0 | 0 | 0 |
| Lmo0448 | 2 | 2 | 2 | 2 | 2 | 0 |
| Lmo0459 | 2 | 2 | 2 | 2 | 2 | 2 |
| Lmo0462 | 2 | 2 | 2 | 2 | 2 | 2 |
| Lmo0466 | 0 | 0 | 0 | 0 | 0 | 0 |
| Lmo0467 | 0 | 0 | 0 | 0 | 0 | 0 |
| Lmo0471 | 0 | 0 | 0 | 0 | 0 | 0 |
| Lmo0472 | 0 | 0 | 0 | 0 | 0 | 0 |
| Lmo0473 | 2 | 2 | 2 | 0 | 2 | 0 |
| Lmo0474 | 2 | 2 | 2 | 2 | 2 | 2 |
| Lmo0493 | 2 | 2 | 2 | 2 | 2 | 2 |
| Lmo0497 | 0 | 0 | 0 | 0 | 0 | 0 |
| Lmo0525 | 0 | 0 | 0 | 0 | 0 | 0 |
| Lmo0549 | 2 | 2 | 2 | 2 | 2 | 2 |
| Lmo0630 | 2 | 2 | 0 | 2 | 0 | 2 |
| Lmo0631 | 2 | 2 | 0 | 2 | 0 | 2 |
| Lmo0632 | 2 | 2 | 0 | 2 | 0 | 2 |
| Lmo0732 | 2 | 2 | 2 | 2 | 2 | 0 |
| Lmo0733 | 2 | 2 | 2 | 2 | 2 | 2 |
| Lmo0734 | 0 | 0 | 0 | 0 | 0 | 0 |
| Lmo0735 | 0 | 0 | 0 | 0 | 0 | 0 |
| Lmo0739 | 0 | 0 | 0 | 0 | 0 | 0 |
| Lmo0750 | 0 | 0 | 0 | 0 | 0 | 0 |
| Lmo0751 | 2 | 2 | 2 | 2 | 0 | 2 |
| Lmo0752 | 2 | 2 | 2 | 2 | 2 | 2 |
| Lmo0753 | 2 | 2 | 2 | 2 | 2 | 2 |
| Lmo0780 | 0 | 0 | 0 | 0 | 0 | 0 |
| Lmo0801 | 2 | 2 | 0 | 0 | 0 | 0 |
| Lmo0804 | 0 | 0 | 0 | 0 | 2 | 2 |
| Lmo0832 | 2 | 2 | 2 | 2 | 2 | 2 |
| Lmo0833 | 2 | 2 | 2 | 2 | 2 | 2 |
| Lmo0834 | 2 | 2 | 2 | 2 | 2 | 2 |
| Lmo0835 | 2 | 2 | 2 | 2 | 2 | 2 |
| Lmo0842 | 2 | 2 | 2 | 2 | 2 | 0 |
| Lmo1030 | 2 | 2 | 2 | 2 | 2 | 2 |
| Lmo1036 | 2 | 2 | 2 | 2 | 2 | 2 |
| Lmo1060 | 0 | 0 | 0 | 0 | 0 | 0 |
| Lmo1061 | 0 | 0 | 0 | 0 | 0 | 0 |
| Lmo1063 | 0 | 0 | 0 | 0 | 0 | 0 |
| Lmo1076 | 0 | 0 | 0 | 0 | 0 | 0 |
| Lmo1077 | 0 | 0 | 0 | 0 | 0 | 0 |
| Lmo1080 | 0 | 0 | 0 | 0 | 0 | 0 |
| Lmo1081 | 0 | 0 | 0 | 0 | 0 | 0 |
| Lmo1082 | 0 | 0 | 0 | 0 | 0 | 0 |
| Lmo1085 | 0 | 0 | 0 | 0 | 0 | 0 |
| Lmo1088 | 0 | 0 | 0 | 0 | 0 | 0 |
| Lmo1090 | 0 | 0 | 0 | 0 | 0 | 0 |
| Lmo1091 | 0 | 0 | 0 | 0 | 0 | 0 |
| Lmo1102 | 2 | 2 | 2 | 2 | 2 | 0 |
| Lmo1115 | 0 | 0 | 0 | 0 | 0 | 0 |
| Lmo1116 | 2 | 2 | 2 | 0 | 2 | 0 |
| Lmo1118 | 0 | 0 | 0 | 0 | 0 | 0 |
| Lmo1119 | 0 | 0 | 0 | 0 | 0 | 0 |
| Lmo1120 | 2 | 2 | 2 | 2 | 0 | 2 |
| Lmo1133 | 2 | 2 | 2 | 2 | 2 | 2 |
| Lmo1135 | 2 | 2 | 2 | 2 | 2 | 2 |
| Lmo1307 | 0 | 0 | 0 | 0 | 0 | 0 |
| Lmo1441 | 2 | 2 | 2 | 2 | 2 | 2 |
| Lmo1451 | 2 | 2 | 2 | 2 | 2 | 2 |
| Lmo1597 | 0 | 0 | 0 | 0 | 2 | 0 |
| Lmo1604 | 2 | 2 | 2 | 2 | 2 | 0 |
| Lmo1648 | 0 | 0 | 0 | 0 | 0 | 0 |
| Lmo1666 | 2 | 2 | 2 | 2 | 2 | 2 |
| Lmo1714 | 2 | 2 | 2 | 2 | 2 | 2 |
| Lmo1968 | 0 | 0 | 0 | 0 | 0 | 0 |
| Lmo1973 | 0 | 0 | 0 | 0 | 0 | 0 |
| Lmo1974 | 0 | 0 | 0 | 0 | 0 | 0 |
| Lmo2026 | 0 | 0 | 0 | 0 | 0 | 0 |
| Lmo2027 | 2 | 2 | 2 | 2 | 2 | 0 |
| Lmo2276 | 2 | 2 | 2 | 2 | 2 | 0 |
| Lmo2277 | 0 | 0 | 0 | 0 | 0 | 0 |
| Lmo2311 | 0 | 0 | 0 | 0 | 0 | 0 |
| Lmo2312 | 2 | 0 | 0 | 2 | 0 | 0 |
| Lmo2324 | 2 | 0 | 0 | 2 | 0 | 0 |
| Lmo2408 | 0 | 0 | 0 | 0 | 0 | 0 |
| Lmo2443 | 2 | 2 | 2 | 2 | 2 | 2 |
| Lmo2576 | 0 | 0 | 0 | 0 | 0 | 0 |
| Lmo2594 | 2 | 2 | 2 | 2 | 2 | 2 |
| Lmo2595 | 2 | 2 | 2 | 2 | 2 | 2 |
| Lmo2671 | 2 | 2 | 2 | 2 | 2 | 2 |
| Lmo2686 | 0 | 0 | 0 | 0 | 0 | 0 |
| Lmo2732 | 2 | 2 | 2 | 2 | 2 | 2 |
| Lmo2736 | 2 | 2 | 2 | 2 | 2 | 2 |
| Lmo2786 | 0 | 0 | 0 | 0 | 0 | 0 |
| Lmo2787 | 0 | 0 | 0 | 0 | 0 | 0 |
| Lmo2807 | 2 | 2 | 0 | 0 | 0 | 0 |
| Lmo2809 | 0 | 2 | 2 | 2 | 2 | 2 |
| Lmo2821 | 2 | 2 | 2 | 2 | 2 | 2 |
| Lin0060 | 0 | 0 | 2 | 2 | 0 | 2 |
| Lin0197 | 0 | 0 | 0 | 0 | 0 | 0 |
| Lin0332 | 0 | 0 | 0 | 0 | 0 | 0 |
| Lin0397 | 0 | 0 | 0 | 0 | 0 | 0 |
| Lin0415 | 2 | 2 | 2 | 2 | 2 | 0 |
| Lin0453 | 2 | 2 | 2 | 2 | 2 | 2 |
| Lin0454 | 2 | 2 | 2 | 2 | 2 | 2 |
| Lin0455 | 0 | 2 | 0 | 2 | 2 | 0 |
| Lin0465 | 0 | 0 | 0 | 0 | 0 | 0 |
| Lin0476 | 2 | 0 | 2 | 2 | 0 | 0 |
| Lin0661 | 0 | 0 | 0 | 0 | 0 | 0 |
| Lin0665 | 2 | 2 | 2 | 2 | 2 | 2 |
| Lin0677 | 0 | 0 | 0 | 0 | 0 | 0 |
| Lin0739 | 0 | 0 | 0 | 0 | 0 | 0 |
| Lin0740 | 2 | 0 | 0 | 0 | 0 | 0 |
| Lin0803 | 0 | 0 | 0 | 0 | 0 | 0 |
| Lin0868 | 0 | 0 | 0 | 0 | 0 | 0 |
| Lin1066 | 0 | 0 | 0 | 0 | 0 | 0 |
| Lin1069 | 0 | 0 | 0 | 0 | 0 | 0 |
| Lin1073 | 2 | 2 | 2 | 2 | 2 | 2 |
| Lin1083 | 0 | 0 | 0 | 0 | 0 | 0 |
| Lin1084 | 2 | 2 | 2 | 2 | 0 | 2 |
| Lin1090 | 2 | 2 | 2 | 2 | 2 | 2 |
| Lin1204 | 2 | 2 | 2 | 2 | 2 | 2 |
| Lin1262 | 2 | 0 | 2 | 2 | 2 | 0 |
| Lin1299 | 2 | 0 | 2 | 2 | 2 | 0 |
| Lin1328 | 2 | 2 | 2 | 2 | 2 | 0 |
| Lin1452 | 0 | 0 | 0 | 0 | 0 | 0 |
| Lin1621 | 0 | 0 | 0 | 0 | 0 | 0 |
| Lin2397 | 0 | 0 | 0 | 0 | 0 | 0 |
| Lin2494 | 2 | 2 | 2 | 0 | 2 | 0 |
| Lin2603 | 0 | 0 | 0 | 0 | 0 | 0 |
| Lin2724 | 0 | 0 | 0 | 0 | 0 | 0 |
| Lin2741 | 0 | 0 | 0 | 0 | 0 | 0 |
| Lin2743 | 0 | 0 | 0 | 0 | 0 | 0 |
| Lin2744 | 0 | 0 | 0 | 0 | 0 | 0 |
| Lin2839 | 0 | 0 | 0 | 0 | 0 | 0 |
| Lin2918 | 0 | 0 | 0 | 0 | 0 | 0 |
| ORF 105 | 2 | 0 | 0 | 0 | 2 | 0 |
| ORF 1390 | 0 | 0 | 0 | 2 | 2 | 0 |
| ORF 1445 | 2 | 2 | 2 | 2 | 2 | 2 |
| ORF 1490 | 2 | 2 | 2 | 2 | 2 | 2 |
| ORF 1491 | 2 | 2 | 2 | 2 | 2 | 2 |
| ORF 1590 | 2 | 2 | 2 | 2 | 2 | 2 |
| ORF 1761 | 2 | 2 | 2 | 2 | 2 | 2 |
| ORF 2017 | 2 | 2 | 2 | 2 | 2 | 2 |
| ORF 2074 | 2 | 2 | 2 | 2 | 2 | 2 |
| ORF 2076 | 2 | 2 | 2 | 2 | 2 | 2 |
| ORF 2110 | 2 | 2 | 2 | 2 | 2 | 2 |
| ORF 2372 | 2 | 2 | 2 | 2 | 2 | 2 |
| ORF 2541 | 2 | 2 | 2 | 2 | 2 | 2 |
| ORF 2579 | 2 | 2 | 2 | 2 | 2 | 2 |
| ORF 2777 | 2 | 2 | 2 | 2 | 2 | 2 |
| ORF 2819 | 2 | 2 | 2 | 2 | 2 | 2 |
| ORF 2847 | 2 | 2 | 0 | 0 | 2 | 0 |
| ORF 2858 | 2 | 2 | 2 | 2 | 2 | 2 |
| ORF 3103 | 0 | 0 | 2 | 2 | 2 | 2 |
| ORF 3184 | 2 | 2 | 2 | 2 | 2 | 0 |
| ORF 3401 | 2 | 2 | 2 | 2 | 2 | 2 |
| ORF 3792 | 2 | 2 | 2 | 2 | 2 | 2 |
| ORF 3840 | 2 | 2 | 2 | 2 | 2 | 2 |
| ORF 4101 | 2 | 2 | 2 | 2 | 2 | 2 |
| ORF 660 | 2 | 2 | 2 | 2 | 2 | 2 |
| ORF 757 | 2 | 2 | 2 | 2 | 2 | 2 |
| ORF 799 | 2 | 2 | 2 | 2 | 2 | 2 |
| ORF 817 | 0 | 0 | 0 | 2 | 2 | 2 |

| Gene | CLIP 74913-4e | CLIP78003-4e | CLIP79455-4e | CLIP86309-4c | CLIP81065-4c | CLIP74911-4c | CLIP78025b-4a | CLIP74908-4a | CLIP71988-4a | CLIP6645-iva/lon-5 | PAM55-5 |
|---|---|---|---|---|---|---|---|---|---|---|---|
| Lmo0017 | 2 | 2 | 2 | 2 | 2 | 2 | 0 | 0 | 0 | 0 | 0 |
| Lmo0036 | 2 | 2 | 2 | 2 | 0 | 0 | 0 | 0 | 0 | 2 | 2 |
| Lmo0037 | 2 | 2 | 2 | 2 | 0 | 0 | 0 | 0 | 0 | 2 | 2 |
| Lmo0066 | 0 | 2 | 0 | 0 | 2 | 2 | 2 | 0 | 0 | 2 | 0 |
| Lmo0067 | 0 | 0 | 0 | 0 | 0 | 0 | 0 | 0 | 0 | 0 | 0 |
| Lmo0068 | 2 | 0 | 2 | 0 | 0 | 0 | 0 | 0 | 0 | 0 | 0 |
| Lmo0069 | 2 | 0 | 2 | 0 | 0 | 0 | 0 | 0 | 0 | 0 | 0 |
| Lmo0071 | 2 | 0 | 2 | 0 | 0 | 0 | 0 | 0 | 0 | 0 | 0 |
| Lmo0072 | 2 | 2 | 2 | 2 | 0 | 0 | 0 | 0 | 0 | 0 | 0 |
| Lmo0079 | 2 | 2 | 2 | 2 | 2 | 2 | 0 | 0 | 0 | 2 | 0 |
| Lmo0080 | 2 | 2 | 2 | 2 | 0 | 0 | 0 | 0 | 0 | 0 | 0 |
| Lmo0081 | 0 | 0 | 0 | 0 | 0 | 0 | 0 | 0 | 0 | 0 | 0 |
| Lmo0082 | 2 | 2 | 2 | 2 | 2 | 2 | 2 | 2 | 2 | 0 | 0 |
| Lmo0083 | 2 | 2 | 2 | 2 | 2 | 2 | 2 | 2 | 2 | 0 | 0 |
| Lmo0094 | 2 | 2 | 2 | 2 | 0 | 0 | 0 | 0 | 0 | 0 | 0 |
| Lmo0106 | 2 | 2 | 2 | 2 | 2 | 2 | 2 | 2 | 2 | 2 | 0 |
| Lmo0141 | 2 | 2 | 2 | 2 | 2 | 0 | 2 | 2 | 2 | 0 | 0 |
| Lmo0143 | 0 | 0 | 0 | 0 | 0 | 0 | 2 | 2 | 2 | 0 | 0 |
| Lmo0144 | 2 | 2 | 2 | 2 | 2 | 0 | 2 | 2 | 2 | 0 | 0 |
| Lmo0147 | 2 | 2 | 2 | 2 | 2 | 2 | 0 | 2 | 2 | 2 | 2 |
| Lmo0151 | 0 | 0 | 0 | 0 | 0 | 0 | 0 | 0 | 0 | 0 | 0 |
| Lmo0171 | 0 | 0 | 0 | 0 | 0 | 0 | 0 | 0 | 0 | 0 | 0 |
| Lmo0172 | 0 | 0 | 0 | 0 | 0 | 0 | 0 | 0 | 0 | 0 | 0 |
| Lmo0252 | 0 | 0 | 0 | 0 | 2 | 2 | 2 | 2 | 2 | 0 | 0 |
| Lmo0253 | 0 | 0 | 0 | 0 | 2 | 2 | 2 | 2 | 2 | 0 | 0 |
| Lmo0304 | 0 | 0 | 0 | 0 | 2 | 0 | 0 | 0 | 0 | 0 | 0 |
| Lmo0310 | 0 | 0 | 0 | 0 | 0 | 0 | 0 | 0 | 0 | 0 | 0 |
| Lmo0320 | 2 | 2 | 2 | 2 | 0 | 0 | 2 | 0 | 0 | 0 | 0 |
| Lmo0333 | 2 | 2 | 2 | 2 | 2 | 2 | 0 | 0 | 0 | 0 | 0 |
| Lmo0334 | 2 | 2 | 2 | 2 | 2 | 2 | 0 | 0 | 0 | 0 | 0 |
| Lmo0338 | 0 | 0 | 0 | 0 | 0 | 0 | 0 | 0 | 0 | 2 | 0 |
| Lmo0378 | 0 | 0 | 0 | 0 | 0 | 0 | 0 | 0 | 0 | 0 | 0 |
| Lmo0379 | 0 | 0 | 0 | 0 | 2 | 2 | 0 | 0 | 0 | 0 | 0 |
| Lmo0380 | 0 | 0 | 0 | 0 | 2 | 2 | 0 | 0 | 0 | 0 | 0 |
| Lmo0381 | 0 | 0 | 0 | 0 | 0 | 0 | 2 | 2 | 2 | 0 | 0 |
| Lmo0409 | 2 | 2 | 2 | 2 | 0 | 0 | 0 | 0 | 0 | 0 | 0 |
| Lmo0419 | 2 | 2 | 2 | 2 | 2 | 2 | 0 | 0 | 0 | 0 | 0 |
| Lmo0440 | 2 | 2 | 2 | 2 | 2 | 2 | 2 | 2 | 2 | 0 | 0 |
| Lmo0444 | 0 | 0 | 0 | 0 | 2 | 2 | 0 | 0 | 0 | 0 | 0 |
| Lmo0448 | 0 | 0 | 2 | 2 | 2 | 2 | 0 | 0 | 2 | 0 | 0 |
| Lmo0459 | 2 | 2 | 2 | 2 | 2 | 0 | 2 | 2 | 2 | 0 | 0 |
| Lmo0462 | 2 | 2 | 2 | 2 | 2 | 0 | 2 | 2 | 2 | 0 | 0 |
| Lmo0466 | 0 | 0 | 0 | 0 | 0 | 0 | 0 | 0 | 0 | 0 | 0 |
| Lmo0467 | 0 | 0 | 0 | 0 | 0 | 0 | 0 | 0 | 0 | 0 | 0 |
| Lmo0471 | 0 | 0 | 0 | 0 | 0 | 0 | 0 | 0 | 0 | 0 | 0 |
| Lmo0472 | 0 | 0 | 0 | 0 | 2 | 2 | 0 | 0 | 0 | 0 | 0 |
| Lmo0473 | 2 | 0 | 2 | 0 | 2 | 2 | 2 | 2 | 2 | 0 | 2 |
| Lmo0474 | 2 | 2 | 2 | 2 | 0 | 0 | 0 | 0 | 0 | 0 | 0 |
| Lmo0493 | 2 | 2 | 2 | 2 | 2 | 0 | 0 | 0 | 0 | 0 | 2 |
| Lmo0497 | 0 | 0 | 0 | 0 | 0 | 0 | 0 | 0 | 0 | 0 | 0 |
| Lmo0525 | 0 | 0 | 0 | 0 | 0 | 0 | 0 | 0 | 0 | 0 | 0 |
| Lmo0549 | 2 | 2 | 2 | 2 | 2 | 2 | 0 | 0 | 0 | 0 | 0 |
| Lmo0630 | 2 | 2 | 2 | 2 | 2 | 0 | 0 | 0 | 0 | 0 | 2 |
| Lmo0631 | 2 | 2 | 2 | 2 | 2 | 0 | 0 | 0 | 0 | 0 | 0 |
| Lmo0632 | 2 | 2 | 2 | 2 | 2 | 0 | 0 | 0 | 0 | 2 | 2 |
| Lmo0732 | 0 | 2 | 2 | 2 | 2 | 2 | 2 | 2 | 2 | 2 | 2 |
| Lmo0733 | 2 | 2 | 2 | 2 | 2 | 2 | 2 | 2 | 2 | 0 | 0 |
| Lmo0734 | 0 | 0 | 0 | 0 | 0 | 0 | 0 | 0 | 0 | 0 | 0 |
| Lmo0735 | 0 | 0 | 0 | 0 | 0 | 0 | 0 | 0 | 0 | 0 | 0 |
| Lmo0739 | 0 | 0 | 0 | 0 | 0 | 0 | 0 | 0 | 0 | 0 | 0 |
| Lmo0750 | 0 | 0 | 0 | 2 | 0 | 0 | 2 | 2 | 2 | 0 | 0 |
| Lmo0751 | 2 | 2 | 2 | 2 | 0 | 0 | 2 | 2 | 2 | 0 | 2 |
| Lmo0752 | 2 | 2 | 2 | 2 | 0 | 0 | 2 | 2 | 2 | 2 | 2 |
| Lmo0753 | 2 | 2 | 2 | 2 | 0 | 0 | 2 | 2 | 2 | 2 | 2 |
| Lmo0780 | 0 | 0 | 0 | 0 | 2 | 2 | 2 | 2 | 2 | 2 | 2 |
| Lmo0801 | 0 | 0 | 2 | 0 | 0 | 0 | 0 | 0 | 2 | 0 | 0 |
| Lmo0804 | 0 | 2 | 0 | 2 | 2 | 2 | 2 | 2 | 2 | 0 | 0 |
| Lmo0832 | 2 | 2 | 2 | 2 | 0 | 0 | 0 | 0 | 0 | 0 | 0 |
| Lmo0833 | 2 | 2 | 2 | 2 | [symbol] | [symbol] | [symbol] | [symbol] | [symbol] | 0 | 0 |
| Lmo0834 | 2 | 2 | 2 | 2 | [symbol] | [symbol] | 0 | 0 | 0 | 0 | 0 |
| Lmo0835 | 2 | 2 | 2 | 2 | 0 | 0 | 0 | 0 | 0 | 0 | 0 |
| Lmo0842 | 0 | 0 | 2 | 2 | 0 | 0 | 0 | 0 | 2 | 0 | 0 |
| Lmo1030 | 2 | 2 | 2 | 2 | 2 | 2 | 0 | 0 | 0 | 2 | 2 |
| Lmo1036 | 2 | 2 | 2 | 2 | 2 | 2 | 0 | 0 | 0 | 2 | 2 |
| Lmo1060 | 0 | 0 | 0 | 0 | 0 | 0 | 0 | 0 | 0 | 0 | 0 |
| Lmo1061 | 0 | 0 | 0 | 0 | 0 | 0 | 0 | 0 | 0 | 0 | 0 |
| Lmo1063 | 0 | 0 | 0 | 0 | 0 | 0 | 0 | 0 | 0 | 0 | 0 |
| Lmo1076 | 0 | 0 | 0 | 0 | 0 | 0 | 0 | 0 | 0 | 0 | 0 |
| Lmo1077 | 0 | 0 | 0 | 0 | 0 | 0 | 0 | 0 | 0 | 0 | 0 |
| Lmo1080 | 0 | 0 | 0 | 0 | 0 | 0 | 0 | 0 | 0 | 0 | 0 |
| Lmo1081 | 0 | 0 | 0 | 0 | 0 | 0 | 0 | 0 | 0 | 0 | 0 |
| Lmo1082 | 0 | 0 | 0 | 0 | 0 | 0 | 0 | 0 | 0 | 0 | 0 |
| Lmo1085 | 0 | 0 | 0 | 0 | 0 | 0 | 0 | 0 | 0 | 0 | 0 |
| Lmo1088 | 0 | 0 | 0 | 0 | 0 | 0 | 0 | 0 | 0 | 0 | 0 |
| Lmo1090 | 0 | 0 | 0 | 0 | 0 | [symbol] | 0 | 0 | 0 | 0 | 0 |
| Lmo1091 | 0 | 0 | 0 | 0 | 0 | 0 | 0 | 0 | 0 | 0 | 0 |
| Lmo1102 | 0 | 2 | 2 | 2 | 2 | 2 | 2 | 2 | 2 | 2 | 2 |
| Lmo1115 | 0 | 2 | 0 | 0 | 2 | 2 | 2 | 0 | 0 | 0 | 0 |
| Lmo1116 | 2 | 0 | 2 | 0 | 0 | 2 | 0 | 0 | 0 | 0 | 2 |
| Lmo1118 | 0 | 0 | 0 | 0 | 0 | 0 | 0 | 0 | 0 | 0 | 0 |
| Lmo1119 | 0 | 0 | 0 | 0 | 0 | 0 | 0 | 0 | 0 | 0 | 0 |
| Lmo1120 | 2 | 2 | 2 | 2 | 0 | 0 | 0 | 0 | 0 | 2 | 2 |
| Lmo1133 | 2 | 2 | 2 | 2 | 2 | 2 | 2 | 2 | 2 | 0 | 0 |
| Lmo1135 | 2 | 2 | 2 | 2 | 2 | 2 | 0 | 0 | 2 | 0 | 0 |
| Lmo1307 | 0 | 0 | 0 | 0 | 2 | 2 | 2 | 2 | 2 | 0 | 2 |
| Lmo1441 | 2 | 2 | 2 | 2 | 0 | 0 | 0 | 0 | 0 | 0 | 0 |
| Lmo1451 | 2 | 2 | 2 | 2 | 0 | 0 | 0 | 0 | 0 | 0 | 0 |
| Lmo1597 | 0 | 0 | 0 | 0 | 0 | 0 | 0 | 0 | 0 | 0 | 2 |
| Lmo1604 | 2 | 0 | 2 | 2 | 2 | 2 | 0 | 0 | 2 | 0 | 2 |
| Lmo1648 | 0 | 0 | 2 | 0 | 0 | 2 | 0 | 0 | 0 | 0 | 0 |
| Lmo1666 | 2 | 2 | 2 | 2 | 0 | 0 | 0 | 0 | 0 | 0 | 0 |
| Lmo1714 | 2 | 2 | 2 | 2 | 0 | 0 | 2 | 2 | 2 | 0 | 0 |
| Lmo1968 | 0 | 0 | 0 | 0 | 0 | 0 | 0 | 0 | 0 | 0 | 0 |
| Lmo1973 | 0 | 0 | 0 | 0 | 0 | 0 | 0 | 0 | 0 | 0 | 0 |
| Lmo1974 | 0 | 0 | 0 | 0 | 0 | 0 | 0 | 0 | 0 | 0 | 0 |
| Lmo2026 | 0 | 0 | 0 | 0 | 0 | 0 | 0 | 0 | 0 | 0 | 0 |
| Lmo2027 | 2 | 2 | 2 | 2 | 2 | 2 | 0 | 0 | 0 | 0 | 0 |
| Lmo2276 | 0 | 2 | 2 | 2 | 0 | 0 | 0 | 0 | 0 | 0 | 0 |
| Lmo2277 | 0 | 0 | 0 | 0 | 2 | 0 | 0 | 0 | 2 | 0 | 0 |
| Lmo2311 | 0 | 0 | 0 | 0 | 2 | 0 | 2 | 2 | 2 | 0 | 0 |
| Lmo2312 | 0 | 2 | 0 | 2 | 0 | 0 | 2 | 2 | 2 | 0 | 0 |
| Lmo2324 | 0 | 2 | 0 | 2 | 0 | 0 | 0 | 2 | 2 | 0 | 0 |
| Lmo2408 | 0 | 0 | 0 | 0 | 0 | 0 | 0 | 0 | 0 | 0 | 0 |
| Lmo2443 | 2 | 2 | 2 | 2 | 0 | 0 | 2 | 2 | 2 | 0 | 0 |
| Lmo2576 | 0 | 0 | 0 | 0 | 2 | 2 | 0 | 0 | 0 | 0 | 0 |
| Lmo2594 | 2 | 2 | 2 | 2 | 2 | 2 | 0 | 0 | 2 | 0 | 0 |
| Lmo2595 | 2 | 2 | 2 | 2 | 0 | 2 | 2 | 2 | 2 | 0 | 0 |
| Lmo2671 | 2 | 2 | 2 | 2 | 0 | 0 | 0 | 0 | 0 | 2 | 0 |
| Lmo2686 | 0 | 0 | 0 | 0 | 2 | 0 | 0 | 0 | 0 | 2 | 0 |
| Lmo2732 | 2 | 2 | 2 | 2 | 2 | 2 | 2 | 2 | 2 | 0 | 0 |
| Lmo2736 | 2 | 2 | 2 | 2 | 2 | 2 | 2 | 2 | 2 | 0 | 0 |
| Lmo2786 | 0 | 0 | 0 | 0 | 2 | 2 | 0 | 0 | 0 | 0 | 0 |
| Lmo2787 | 0 | 0 | 0 | 0 | 2 | 2 | 0 | 0 | 0 | 0 | 0 |
| Lmo2807 | 2 | 0 | 2 | 0 | 0 | 0 | 0 | 2 | 2 | 0 | 0 |
| Lmo2809 | 0 | 2 | 0 | 2 | 0 | 0 | 0 | 0 | 0 | 0 | 0 |
| Lmo2821 | 2 | 2 | 2 | 2 | 2 | 2 | 0 | 0 | 0 | 0 | 0 |
| Lin0060 | 0 | 2 | 0 | 2 | 0 | 0 | 0 | 0 | 0 | 0 | 0 |
| Lin0197 | 0 | 0 | 0 | 0 | 0 | 0 | 0 | 0 | 0 | 2 | 2 |
| Lin0332 | 0 | 0 | 0 | 0 | 0 | 0 | 0 | 0 | 0 | 0 | 0 |
| Lin0397 | 0 | 0 | 0 | 0 | 0 | 0 | 0 | 0 | 0 | 0 | 0 |
| Lin0415 | 0 | 2 | 2 | 2 | 2 | 2 | 0 | 0 | 0 | 0 | 0 |
| Lin0453 | 2 | 2 | 2 | 2 | 0 | 0 | 0 | 0 | 0 | 0 | 0 |
| Lin0454 | 2 | 2 | 2 | 2 | 2 | 2 | 2 | 2 | 2 | 2 | 0 |
| Lin0455 | 2 | 0 | 2 | 2 | 2 | 0 | 2 | 2 | 2 | 2 | 0 |
| Lin0465 | 0 | 0 | 0 | 0 | 0 | 0 | 0 | 0 | 0 | 0 | 0 |
| Lin0476 | 0 | 0 | 0 | 0 | 2 | 2 | 0 | 0 | 0 | 0 | 0 |
| Lin0661 | 0 | 0 | 0 | 0 | 2 | 0 | 0 | 0 | 0 | 0 | 0 |
| Lin0665 | 2 | 2 | 2 | 2 | 2 | 2 | 2 | 2 | 2 | 0 | 0 |
| Lin0677 | 0 | 0 | 0 | 0 | 2 | 2 | 2 | 2 | 2 | 0 | 0 |
| Lin0739 | 0 | 0 | 0 | 0 | 0 | 0 | 0 | 0 | 0 | 0 | 0 |
| Lin0740 | 0 | 2 | 0 | 2 | 2 | 2 | 2 | 0 | 2 | 0 | 0 |
| Lin0803 | 0 | 0 | 0 | 0 | 0 | 0 | 0 | 0 | 0 | 0 | 0 |
| Lin0868 | 0 | 0 | 0 | 0 | 0 | 0 | 0 | 0 | 0 | 0 | 0 |
| Lin1066 | 0 | 0 | 0 | 2 | 0 | 0 | 0 | 0 | 0 | 0 | 0 |
| Lin1069 | 0 | 0 | 0 | 0 | 0 | 0 | 0 | 0 | 0 | 0 | 0 |
| Lin1073 | 2 | 2 | 2 | 2 | 2 | 2 | 2 | 2 | 2 | 2 | 2 |
| Lin1083 | 0 | 0 | 0 | 0 | 0 | 0 | 0 | 0 | 0 | 0 | 0 |
| Lin1084 | 2 | 2 | 2 | 2 | 0 | 0 | 0 | 0 | 0 | 2 | 0 |
| Lin1090 | 2 | 2 | 2 | 2 | 2 | 2 | 2 | 0 | 2 | 0 | 2 |
| Lin1204 | 2 | 2 | 2 | 2 | 0 | 0 | 2 | 2 | 2 | 0 | 0 |
| Lin1262 | 0 | 2 | 2 | 2 | 2 | 2 | 2 | 0 | 2 | 0 | 0 |
| Lin1299 | 0 | 2 | 2 | 2 | 2 | 2 | 2 | 0 | 2 | 0 | 0 |
| Lin1328 | 0 | 2 | 2 | 2 | 2 | 2 | 2 | 0 | 2 | 2 | 2 |
| Lin1452 | 0 | 0 | 0 | 0 | 0 | 0 | 0 | 0 | 0 | 0 | 0 |
| Lin1621 | 0 | 0 | 0 | 0 | 0 | 0 | 2 | 2 | 2 | 0 | 0 |
| Lin2397 | 0 | 2 | 0 | 2 | 0 | 0 | 0 | 0 | 0 | 0 | 0 |
| Lin2494 | 0 | 0 | 0 | 0 | 2 | 2 | 2 | 2 | 2 | 0 | 0 |
| Lin2603 | 0 | 0 | 0 | 0 | 0 | 0 | 2 | 2 | 2 | 0 | 0 |
| Lin2724 | 0 | 0 | 0 | 0 | 2 | 0 | 2 | 2 | 2 | 2 | 0 |
| Lin2741 | 0 | 0 | 0 | 0 | 0 | 0 | 0 | 0 | 0 | 0 | 0 |
| Lin2743 | 0 | 0 | 0 | 0 | 2 | 0 | 0 | 0 | 0 | 0 | 0 |
| Lin2744 | 0 | 0 | 0 | 2 | 2 | 2 | 0 | 0 | 0 | 0 | 0 |
| Lin2839 | 0 | 0 | 0 | 0 | 0 | 0 | 0 | 0 | 0 | 0 | 0 |
| Lin2918 | 0 | 0 | 0 | 0 | 0 | 0 | 0 | 0 | 0 | 0 | 0 |
| ORF 105 | 2 | 0 | 2 | 0 | 0 | 0 | 0 | 0 | 0 | 0 | 2 |
| ORF 1390 | 0 | 2 | 2 | 2 | 0 | 0 | 2 | 0 | 2 | 0 | 2 |
| ORF 1445 | 2 | 2 | 2 | 2 | 2 | 2 | 2 | 2 | 2 | 0 | 2 |
| ORF 1490 | 2 | 2 | 2 | 2 | 2 | 2 | 0 | 0 | 0 | 2 | 0 |
| ORF 1491 | 2 | 2 | 2 | 2 | 2 | 2 | 0 | 0 | 0 | 2 | 0 |
| ORF 1590 | 2 | 2 | 2 | 2 | 2 | 2 | 0 | 0 | 0 | 0 | 0 |
| ORF 1761 | 2 | 2 | 2 | 2 | 0 | 0 | 0 | 0 | 0 | 0 | 0 |
| ORF 2017 | 2 | 2 | 2 | 2 | 2 | 2 | 0 | 0 | 2 | 0 | 2 |
| ORF 2074 | 2 | 2 | 2 | 2 | 0 | 0 | 2 | 2 | 2 | 2 | 2 |
| ORF 2076 | 2 | 2 | 2 | 2 | 0 | 0 | 2 | 2 | 2 | 2 | 2 |
| ORF 2110 | 2 | 2 | 2 | 2 | 0 | 0 | 0 | 0 | 0 | 2 | 2 |
| ORF 2372 | 2 | 2 | 2 | 2 | 0 | 0 | 0 | 0 | 0 | 0 | 2 |
| ORF 2541 | 2 | 2 | 2 | 2 | 0 | 0 | 0 | 0 | 0 | 0 | 0 |
| ORF 2579 | 2 | 2 | 2 | 2 | 0 | 0 | 2 | 2 | 2 | 2 | 2 |
| ORF 2777 | 2 | 2 | 2 | 2 | 0 | 0 | 0 | 0 | 0 | 0 | 2 |
| ORF 2819 | 2 | 2 | 2 | 2 | 0 | 0 | 0 | 0 | 0 | 0 | 0 |
| ORF 2847 | 2 | 0 | 2 | 0 | 0 | 0 | 2 | 2 | 2 | 0 | 0 |
| ORF 2858 | 2 | 2 | 2 | 2 | 2 | 2 | 0 | 0 | 2 | 2 | 2 |
| ORF 3103 | 0 | 2 | 0 | 2 | 0 | 0 | 0 | 0 | 0 | 0 | 0 |
| ORF 3184 | 2 | 2 | 2 | 2 | 0 | 2 | 0 | 0 | 2 | 0 | 2 |
| ORF 3401 | 2 | 2 | 2 | 2 | 0 | 0 | 0 | 0 | 2 | 2 | 2 |
| ORF 3792 | 2 | 2 | 2 | 2 | 2 | 2 | 2 | 2 | 2 | 0 | 2 |
| ORF 3840 | 2 | 2 | 2 | 2 | 0 | 0 | 0 | 0 | 0 | 0 | 0 |
| ORF 4101 | 2 | 2 | 2 | 2 | 2 | 2 | 0 | 0 | 2 | 2 | 2 |
| ORF 660 | 2 | 2 | 2 | 2 | 0 | 0 | 0 | 0 | 0 | 0 | 0 |
| ORF 757 | 2 | 2 | 2 | 2 | 0 | 0 | 0 | 0 | 0 | 0 | 0 |
| ORF 799 | 2 | 2 | 2 | 2 | 0 | 0 | 0 | 0 | 0 | 0 | 0 |
| ORF 817 | 0 | 2 | 0 | 2 | 0 | 2 | 0 | 0 | 0 | 2 | 0 |

| | CLIP86784- | CLIP12065b- | CLIP88111b- | CLIP | | CLIP87973- | CLIP86579- | | |
|---|---|---|---|---|---|---|---|---|---|
| Gene | iva/lon-5 | iva/lon- [symbol] | iva/iva-5 | 74914-iva/iva-5 | CLIP87073-wel-6a | wel-4c | sel-1/2b | CLIP73021-sel-1/2b | CLIP88307-inno-6b |
| Lmo0017 | 0 | 0 | 0 | 0 | 0 | 0 | 0 | 0 | 0 |
| Lmo0036 | 2 | 2 | 2 | 2 | 0 | 0 | 2 | 0 | 0 |
| Lmo0037 | 2 | 2 | 2 | 2 | 0 | 0 | 2 | 0 | 0 |
| Lmo0066 | 0 | 2 | 2 | 0 | 0 | 0 | 0 | 0 | 0 |
| Lmo0067 | 0 | 0 | 0 | 0 | 0 | 0 | 0 | 0 | 0 |
| Lmo0068 | 0 | 0 | 0 | 0 | 0 | 0 | 0 | 0 | 0 |
| Lmo0069 | 0 | 0 | 0 | 0 | 0 | 0 | 0 | 0 | 0 |
| Lmo0071 | 0 | 0 | 0 | 0 | 0 | 0 | 0 | 0 | 0 |
| Lmo0072 | 0 | 0 | 0 | 0 | 0 | 0 | 0 | 0 | 0 |
| Lmo0079 | 0 | 2 | 0 | 0 | 0 | 0 | 2 | 0 | 0 |
| Lmo0080 | 0 | 0 | 0 | 0 | 0 | 0 | 2 | 0 | 0 |
| Lmo0081 | 0 | 0 | 0 | 0 | 0 | 0 | 0 | 0 | 0 |
| Lmo0082 | 0 | 0 | 0 | 0 | 0 | 0 | 0 | 0 | 0 |
| Lmo0083 | 0 | 0 | 0 | 0 | 0 | 0 | 0 | 0 | 0 |
| Lmo0094 | 0 | 0 | 0 | 0 | 0 | 0 | 0 | 0 | 0 |
| Lmo0106 | 0 | 2 | 0 | 0 | 2 | 0 | 2 | 2 | 0 |
| Lmo0141 | 0 | 0 | 0 | 0 | 0 | 0 | 0 | 0 | 0 |
| Lmo0143 | 0 | 0 | 0 | 0 | 0 | 0 | 0 | 0 | 0 |
| Lmo0144 | 0 | 0 | 0 | 0 | 0 | 0 | 0 | 0 | 0 |
| Lmo0147 | 2 | 2 | 0 | 2 | 0 | 2 | 0 | 2 | 2 |
| Lmo0151 | 0 | 0 | 0 | 0 | 0 | 0 | 0 | 0 | 0 |
| Lmo0171 | 0 | 0 | 0 | 0 | 0 | 0 | 0 | 0 | 0 |
| Lmo0172 | 0 | 0 | 0 | 0 | 0 | 0 | 0 | 0 | 0 |
| Lmo0252 | 0 | 0 | 0 | 0 | 2 | 2 | 0 | 2 | 0 |
| Lmo0253 | 0 | 0 | 0 | 0 | 0 | 0 | 0 | 2 | 0 |
| Lmo0304 | 0 | 0 | 0 | 0 | 0 | 2 | 0 | 0 | 0 |
| Lmo0310 | 0 | 0 | 0 | 0 | 0 | 0 | 0 | 0 | 0 |
| Lmo0320 | 0 | 0 | 0 | 0 | 0 | 0 | 0 | 0 | 0 |
| Lmo0333 | 0 | 0 | 0 | 0 | 0 | 0 | 0 | 0 | 0 |
| Lmo0334 | 0 | 0 | 0 | 0 | 2 | 0 | 0 | 0 | 0 |
| Lmo0338 | 0 | 2 | 0 | 0 | 0 | 0 | 0 | 0 | 0 |
| Lmo0378 | 0 | 0 | 0 | 0 | 0 | 0 | 0 | 0 | 0 |
| Lmo0379 | 0 | 0 | 0 | 0 | 0 | 0 | 0 | 0 | 0 |
| Lmo0380 | 0 | 0 | 0 | 0 | 0 | 0 | 0 | 0 | 0 |
| Lmo0381 | 0 | 0 | 0 | 0 | 0 | 0 | 0 | 0 | 0 |
| Lmo0409 | 0 | 0 | 0 | 0 | 0 | 0 | 0 | 0 | 0 |
| Lmo0419 | 0 | 0 | 0 | 0 | 2 | 2 | 0 | 0 | 0 |
| Lmo0440 | 0 | 0 | 0 | 0 | 0 | 0 | 0 | 0 | 0 |
| Lmo0444 | 0 | 0 | 0 | 0 | 2 | 2 | 0 | 0 | 0 |
| Lmo0448 | 0 | 0 | 0 | 0 | 2 | 2 | 0 | 0 | 2 |
| Lmo0459 | 0 | 0 | 0 | 0 | 0 | 0 | 0 | 0 | 0 |
| Lmo0462 | 0 | 0 | 0 | 0 | 0 | 0 | 0 | 0 | 0 |
| Lmo0466 | 0 | 0 | 0 | 0 | 0 | 0 | 0 | 0 | 0 |
| Lmo0467 | 0 | 0 | 0 | 0 | 0 | 0 | 0 | 0 | 0 |
| Lmo0471 | 0 | 0 | 0 | 0 | 0 | 0 | 0 | 0 | 0 |
| Lmo0472 | 0 | 0 | 0 | 0 | 2 | 0 | 0 | 0 | 2 |
| Lmo0473 | 2 | 0 | 2 | 2 | 2 | 0 | 0 | 0 | 0 |
| Lmo0474 | 0 | 0 | 0 | 0 | 0 | 0 | 0 | 0 | 0 |
| Lmo0493 | 2 | 0 | 2 | 2 | 0 | 2 | 2 | 0 | 0 |
| Lmo0497 | 0 | 0 | 0 | 0 | 0 | 0 | 0 | 0 | 0 |
| Lmo0525 | 0 | 0 | 0 | 0 | 2 | 0 | 0 | 0 | 0 |
| Lmo0549 | 0 | 0 | 0 | 0 | 0 | 0 | 0 | 0 | 0 |
| Lmo0630 | 2 | 0 | 0 | 2 | 2 | 2 | 0 | 0 | 0 |
| Lmo0631 | 0 | 0 | 0 | 0 | 2 | 2 | 0 | 0 | 0 |
| Lmo0632 | 2 | 2 | 2 | 2 | 2 | 2 | 0 | 0 | 0 |
| Lmo0732 | 2 | 0 | 0 | 0 | 0 | 2 | 0 | 0 | 2 |
| Lmo0733 | 0 | 0 | 0 | 0 | 0 | 0 | 0 | 0 | 0 |
| Lmo0734 | 0 | 0 | 0 | 0 | 2 | 2 | 0 | 0 | 0 |
| Lmo0735 | 0 | 0 | 0 | 2 | 2 | 0 | 0 | 0 | 0 |
| Lmo0739 | 0 | 0 | 0 | 0 | 0 | 0 | 0 | 0 | 0 |
| Lmo0750 | 0 | 0 | 0 | 0 | 0 | 0 | 0 | 0 | 0 |
| Lmo0751 | 0 | 0 | 0 | 0 | 0 | 0 | 0 | 0 | 0 |
| Lmo0752 | 2 | 2 | 2 | 2 | 0 | 0 | 0 | 0 | 0 |
| Lmo0753 | 2 | 2 | 2 | 2 | 0 | 0 | 0 | 0 | 0 |
| Lmo0780 | 0 | 0 | 0 | 0 | 0 | 2 | 0 | 0 | 0 |
| Lmo0801 | 0 | 0 | 0 | 0 | 0 | 2 | 0 | 2 | 2 |
| Lmo0804 | 0 | 0 | 0 | 0 | 0 | 0 | 0 | 0 | 0 |
| Lmo0832 | 0 | 0 | 0 | 0 | 0 | 0 | 0 | 0 | 0 |
| Lmo0833 | 0 | 0 | 0 | 0 | 0 | 0 | 0 | 0 | 0 |
| Lmo0834 | 0 | 0 | 0 | 0 | 0 | 0 | 0 | 0 | 0 |
| Lmo0835 | 0 | 0 | 0 | 0 | 0 | 0 | 0 | 0 | 0 |
| Lmo0842 | 0 | 0 | 0 | 2 | 2 | 2 | 0 | 0 | 2 |
| Lmo1030 | 2 | 2 | 2 | 2 | 0 | 0 | 0 | 0 | 0 |
| Lmo1036 | 2 | 2 | 2 | 2 | 0 | 0 | 0 | 0 | 0 |
| Lmo1060 | 0 | 0 | 0 | 0 | 2 | 2 | 0 | 0 | 0 |
| Lmo1061 | 0 | 0 | 0 | 0 | 2 | 0 | 0 | 0 | 0 |
| Lmo1063 | 0 | 0 | 0 | 0 | 2 | 2 | 0 | 0 | 0 |
| Lmo1076 | 0 | 0 | 0 | 0 | 0 | 0 | 2 | 2 | 0 |
| Lmo1077 | 0 | 0 | 0 | 0 | 0 | 0 | 2 | 2 | 0 |
| Lmo1080 | 0 | 0 | 0 | 0 | 0 | 0 | 0 | 2 | 0 |
| Lmo1081 | 0 | 0 | 0 | 0 | 0 | 0 | 2 | 2 | 0 |
| Lmo1082 | 0 | 0 | 0 | 0 | 0 | 0 | 2 | 2 | 0 |
| Lmo1085 | 0 | 0 | 0 | 0 | 0 | 0 | 0 | 0 | 0 |
| Lmo1088 | 0 | 0 | 0 | 0 | 0 | 0 | 0 | 2 | 0 |
| Lmo1090 | 0 | 0 | 0 | 0 | 0 | 0 | 2 | 2 | 0 |
| Lmo1091 | 0 | 0 | 0 | 0 | 0 | 0 | 2 | 2 | 0 |
| Lmo1102 | 2 | 2 | 2 | 2 | 0 | 0 | 2 | 0 | 0 |
| Lmo1115 | 0 | 2 | 2 | 0 | 0 | 0 | 2 | 0 | 0 |
| Lmo1116 | 2 | 0 | 2 | 2 | 0 | 0 | 0 | 0 | 0 |
| Lmo1118 | 0 | 0 | 0 | 2 | 0 | 0 | 0 | 0 | 0 |
| Lmo1119 | 0 | 0 | 0 | 0 | 0 | 0 | 0 | 0 | 0 |
| Lmo1120 | 2 | 0 | 0 | 2 | 2 | 0 | 0 | 2 | 2 |
| Lmo1133 | 0 | 0 | 0 | 0 | 0 | 0 | 0 | 0 | 0 |
| Lmo1135 | 0 | 0 | 0 | 0 | 0 | 0 | 0 | 0 | 0 |
| Lmo1307 | 2 | 0 | 0 | 0 | 2 | 0 | 0 | 0 | 0 |
| Lmo1441 | 0 | 0 | 0 | 0 | 0 | 0 | 0 | 0 | 0 |
| Lmo1451 | 0 | 0 | 0 | 0 | 0 | 0 | 0 | 0 | 0 |
| Lmo1597 | 2 | 0 | 2 | 2 | 2 | 2 | 0 | 0 | 0 |
| Lmo1604 | 0 | 0 | 0 | 2 | 0 | 0 | 0 | 0 | 2 |
| Lmo1648 | 2 | 0 | 0 | 0 | 0 | 2 | 0 | 0 | 0 |
| Lmo1666 | 0 | 0 | 0 | 0 | 0 | 0 | 0 | 2 | 0 |
| Lmo1714 | 0 | 0 | 0 | 0 | 0 | 0 | 0 | 0 | 0 |
| Lmo1968 | 0 | 0 | 0 | 0 | 0 | 0 | 0 | 0 | 0 |
| Lmo1973 | 0 | 0 | 0 | 0 | 0 | 0 | 0 | 0 | 0 |
| Lmo1974 | 0 | 0 | 0 | 0 | 0 | 0 | 0 | 0 | 0 |
| Lmo2026 | 0 | 0 | 0 | 0 | 0 | 0 | 0 | 0 | 0 |
| Lmo2027 | 0 | 0 | 0 | 0 | 0 | 0 | 0 | 0 | 0 |
| Lmo2276 | 0 | 0 | 0 | 0 | 0 | 0 | 0 | 0 | 0 |
| Lmo2277 | 0 | 0 | 0 | 2 | 0 | 0 | 0 | 0 | 2 |
| Lmo2311 | 0 | 0 | 0 | 0 | 0 | 0 | 0 | 0 | 0 |
| Lmo2312 | 0 | 0 | 0 | 0 | 0 | 2 | 0 | 0 | 0 |
| Lmo2324 | 0 | 0 | 0 | 0 | 0 | 2 | 0 | 0 | 0 |
| Lmo2408 | 0 | 0 | 0 | 0 | 2 | 2 | 0 | 0 | 0 |
| Lmo2443 | 0 | 0 | 0 | 0 | 0 | 0 | 0 | 0 | 0 |
| Lmo2576 | 0 | 0 | 0 | 0 | 0 | 0 | 0 | 0 | 0 |
| Lmo2594 | 0 | 0 | 0 | 0 | 0 | 0 | 0 | 0 | 2 |
| Lmo2595 | 0 | 0 | 0 | 0 | 0 | 0 | 0 | 0 | 0 |
| Lmo2671 | 0 | 0 | 0 | 0 | 2 | 2 | 0 | 2 | 0 |
| Lmo2686 | 0 | 0 | 0 | 0 | 0 | 0 | 0 | 0 | 0 |
| Lmo2732 | 0 | 0 | 0 | 0 | 0 | 0 | 0 | 0 | 0 |
| Lmo2736 | 0 | 0 | 0 | 0 | 0 | 0 | 0 | 0 | 0 |
| Lmo2786 | 0 | 0 | 0 | 0 | 0 | 0 | 0 | 0 | 0 |
| Lmo2787 | 0 | 0 | 0 | 0 | 0 | 0 | 0 | 0 | 0 |
| Lmo2807 | 0 | 0 | 0 | 2 | 0 | 0 | 0 | 2 | 2 |
| Lmo2809 | 0 | 0 | 0 | 0 | 0 | 0 | 0 | 2 | 0 |
| Lmo2821 | 0 | 0 | 0 | 0 | 0 | 0 | 0 | 0 | 0 |
| Lin0060 | 0 | 0 | 0 | 0 | 0 | 0 | 0 | 0 | 2 |
| Lin0197 | 2 | 2 | 2 | 2 | 2 | 2 | 2 | 2 | 2 |
| Lin0332 | 0 | 0 | 0 | 0 | 0 | 0 | 0 | 0 | 2 |
| Lin0397 | 0 | 0 | 0 | 0 | 0 | 0 | 0 | 0 | 2 |
| Lin0415 | 0 | 0 | 0 | 0 | 2 | 2 | 0 | 2 | 2 |
| Lin0453 | 0 | 0 | 0 | 0 | 2 | 2 | 0 | 0 | 2 |
| Lin0454 | 0 | 0 | 0 | 0 | 2 | 2 | 0 | 0 | 2 |
| Lin0455 | 0 | 2 | 0 | 0 | 0 | 2 | 0 | 0 | 2 |
| Lin0465 | 0 | 0 | 0 | 0 | 0 | 0 | 0 | 0 | 2 |
| Lin0476 | 0 | 0 | 0 | 0 | 0 | 0 | 0 | 0 | 2 |
| Lin0661 | 0 | 0 | 0 | 0 | 0 | 0 | 0 | 0 | 2 |
| Lin0665 | 0 | 0 | 0 | 0 | 0 | 2 | 2 | 2 | 2 |
| Lin0677 | 0 | 0 | 0 | 0 | 0 | 0 | 0 | 0 | 2 |
| Lin0739 | 0 | 0 | 0 | 0 | 0 | 0 | 0 | 0 | 2 |
| Lin0740 | 0 | 0 | 0 | 0 | 0 | 0 | 0 | 0 | 2 |
| Lin0803 | 0 | 0 | 0 | 0 | 0 | 0 | 0 | 0 | 2 |
| Lin0868 | 0 | 0 | 0 | 0 | 0 | 0 | 0 | 0 | 2 |
| Lin1066 | 0 | 0 | 0 | 0 | 0 | 0 | 0 | 0 | 2 |
| Lin1069 | 0 | 0 | 0 | 0 | 0 | 0 | 0 | 0 | 2 |
| Lin1073 | 2 | 2 | 0 | 2 | 2 | 0 | 0 | 0 | 2 |
| Lin1083 | 0 | 0 | 0 | 0 | 0 | 0 | 0 | 0 | 2 |
| Lin1084 | 0 | 0 | 0 | 0 | 2 | 0 | 2 | 2 | 2 |
| Lin1090 | 2 | 0 | 2 | 2 | 0 | 0 | 0 | 0 | 2 |
| Lin1204 | 0 | 0 | 0 | 0 | 0 | 0 | 0 | 0 | 2 |
| Lin1262 | 0 | 0 | 0 | 0 | 0 | 0 | 0 | 0 | 2 |
| Lin1299 | 0 | 0 | 2 | 2 | 0 | 0 | 2 | 0 | 2 |
| Lin1328 | 2 | 2 | 2 | 0 | 0 | 0 | 2 | 0 | 2 |
| Lin1452 | 0 | 0 | 0 | 0 | 0 | 0 | 0 | 0 | 2 |
| Lin1621 | 0 | 0 | 0 | 0 | 0 | 0 | 0 | 0 | 2 |
| Lin2397 | 0 | 0 | 0 | 0 | 0 | 2 | 2 | 0 | 2 |
| Lin2494 | 0 | 0 | 0 | 0 | 2 | 2 | 2 | 0 | 2 |
| Lin2603 | 0 | 0 | 0 | 0 | 0 | 0 | 2 | 0 | 2 |
| Lin2724 | 0 | 0 | 0 | 0 | 0 | 0 | 0 | 0 | 2 |
| Lin2741 | 0 | 0 | 0 | 0 | 0 | 0 | 0 | 0 | 2 |
| Lin2743 | 0 | 2 | 0 | 0 | 2 | 0 | 2 | 0 | 2 |
| Lin2744 | 0 | 2 | 0 | 0 | 2 | 0 | 2 | 0 | 2 |
| Lin2839 | 0 | 0 | 0 | 0 | 0 | 0 | 0 | 0 | 2 |
| Lin2918 | 0 | 0 | 0 | 0 | 0 | 0 | 0 | 0 | 2 |
| ORF 105 | 2 | 0 | 2 | 2 | 0 | 0 | 0 | 0 | 0 |
| ORF 1390 | 0 | 0 | 0 | 2 | 0 | 0 | 2 | 0 | 0 |
| ORF 1445 | 2 | 0 | 0 | 2 | 0 | 0 | 0 | 0 | 0 |
| ORF 1490 | 0 | 2 | 0 | 0 | 2 | 2 | 2 | 2 | 0 |
| ORF 1491 | 0 | 2 | 0 | 0 | 2 | 2 | 2 | 2 | 0 |
| ORF 1590 | 0 | 0 | 0 | 0 | 0 | 0 | 0 | 0 | 0 |
| ORF 1761 | 0 | 0 | 0 | 0 | 0 | 0 | 0 | 0 | 0 |
| ORF 2017 | 2 | 0 | 0 | 2 | 0 | 0 | 0 | 0 | 0 |
| ORF 2074 | 2 | 2 | 2 | 2 | 2 | 2 | 2 | 0 | 0 |
| ORF 2076 | 2 | 2 | 2 | 2 | 2 | 2 | 2 | 0 | 0 |
| ORF 2110 | 2 | 0 | 0 | 2 | 0 | 2 | 0 | 0 | 0 |
| ORF 2372 | 0 | 0 | 0 | 2 | 2 | 2 | 0 | 0 | 0 |
| ORF 2541 | 0 | 0 | 0 | 0 | 0 | 0 | 0 | 0 | 0 |
| ORF 2579 | 2 | 2 | 2 | 2 | 2 | 2 | 2 | 0 | 0 |
| ORF 2777 | 2 | 0 | 0 | 2 | 0 | 2 | 2 | 2 | 0 |
| ORF 2819 | 0 | 0 | 0 | 0 | 0 | 0 | 0 | 0 | 0 |
| ORF 2847 | 0 | 0 | 0 | 0 | 0 | 0 | 0 | 0 | 0 |
| ORF 2858 | 2 | 0 | 0 | 0 | 0 | 2 | 0 | 2 | 2 |
| ORF 3103 | 0 | 0 | 0 | 0 | 0 | 0 | 0 | 0 | 0 |
| ORF 3184 | 0 | 0 | 0 | 2 | 0 | 2 | 0 | 0 | 0 |
| ORF 3401 | 0 | 0 | 0 | 0 | 0 | 0 | 0 | 0 | 0 |
| ORF 3792 | 0 | 0 | 0 | 0 | 0 | 0 | 0 | 0 | 0 |
| ORF 3840 | 0 | 0 | 0 | 2 | 0 | 0 | 0 | 0 | 0 |
| ORF 4101 | 2 | 0 | 0 | 2 | 2 | 2 | 0 | 0 | 2 |
| ORF 660 | 0 | 0 | 0 | 0 | 0 | 0 | 0 | 0 | 0 |
| ORF 757 | 0 | 0 | 0 | 0 | 0 | 0 | 0 | 0 | 0 |
| ORF 799 | 0 | 0 | 0 | 0 | 2 | 0 | 0 | 0 | 0 |
| ORF 817 | 0 | 2 | 0 | 0 | 0 | 0 | 0 | 0 | 0 |

| Gene | CLIP86490-inno | CLIP88566-inno-6a | CLIP11262-inno-6a | CLIP 74915-inno-6a | CLIP 74916-inno-6b | CLIP71989-inno-6b | CLIP71990-inno-6b | CLIP73019-gravi | CLIP12515-murrayi |
|---|---|---|---|---|---|---|---|---|---|
| Lmo0017 | 0 | 0 | 0 | 0 | 0 | 0 | 0 | 0 | 0 |
| Lmo0036 | 0 | 0 | 0 | 0 | 0 | 0 | 0 | 2 | 2 |
| Lmo0037 | 0 | 0 | 0 | 0 | 0 | 0 | 0 | 0 | 0 |
| Lmo0066 | 0 | 0 | 0 | 0 | 0 | 2 | 2 | 0 | 0 |
| Lmo0067 | 0 | 0 | 0 | 0 | 0 | 2 | 2 | 0 | 0 |
| Lmo0068 | 0 | 0 | 0 | 0 | 0 | 2 | 2 | 0 | 0 |
| Lmo0069 | 0 | 0 | 0 | 0 | 0 | 0 | 0 | 0 | 0 |
| Lmo0071 | 0 | 0 | 0 | 0 | 0 | 0 | 0 | 0 | 0 |
| Lmo0072 | 2 | 0 | 0 | 0 | 2 | 0 | 0 | 0 | 0 |
| Lmo0079 | 0 | 0 | 0 | 0 | 0 | 0 | 0 | 0 | 0 |
| Lmo0080 | 0 | 0 | 0 | 0 | 0 | 0 | 0 | 0 | 0 |
| Lmo0081 | 0 | 0 | 0 | 0 | 0 | 0 | 0 | 0 | 0 |
| Lmo0082 | 0 | 0 | 0 | 0 | 0 | 0 | 0 | 0 | 0 |
| Lmo0083 | 0 | 0 | 0 | 0 | 0 | 0 | 0 | 0 | 0 |
| Lmo0094 | 0 | 0 | 0 | 0 | 0 | 0 | 0 | 0 | 0 |
| Lmo0106 | 0 | 0 | 0 | 0 | 0 | 0 | 0 | 0 | 0 |
| Lmo0141 | 0 | 0 | 0 | 0 | 0 | 0 | 0 | 0 | 0 |
| Lmo0143 | 0 | 0 | 0 | 0 | 0 | 0 | 0 | 0 | 0 |
| Lmo0144 | 0 | 0 | 0 | 0 | 0 | 0 | 0 | 0 | 0 |
| Lmo0147 | 2 | 2 | 0 | 0 | 0 | 2 | 2 | 0 | 0 |
| Lmo0151 | 0 | 0 | 0 | 0 | 0 | 0 | 0 | 0 | 0 |
| Lmo0171 | 0 | 0 | 0 | 0 | 0 | 0 | 0 | 0 | 0 |
| Lmo0172 | 0 | 0 | 0 | 0 | 0 | 0 | 0 | 0 | 0 |
| Lmo0252 | 0 | 0 | 0 | 0 | 0 | 0 | 0 | 0 | 0 |
| Lmo0253 | 0 | 0 | 0 | 0 | 0 | 0 | 0 | 0 | 0 |
| Lmo0304 | 2 | 2 | 0 | 0 | 0 | 2 | 2 | 0 | 0 |
| Lmo0310 | 0 | 0 | 0 | 0 | 0 | 0 | 0 | 0 | 0 |
| Lmo0320 | 0 | 0 | 0 | 0 | 0 | 0 | 2 | 0 | 0 |
| Lmo0333 | 0 | 0 | 0 | 0 | 0 | 0 | 0 | 0 | 0 |
| Lmo0334 | 0 | 0 | 0 | 0 | 0 | 0 | 0 | 0 | 0 |
| Lmo0338 | 0 | 2 | 0 | 0 | 0 | 0 | 0 | 0 | 2 |
| Lmo0378 | 2 | 0 | 0 | 0 | 2 | 2 | 2 | 0 | 0 |
| Lmo0379 | 2 | 0 | 0 | 0 | 2 | 0 | 0 | 0 | 0 |
| Lmo0380 | 2 | 0 | 0 | 0 | 2 | 0 | 0 | 0 | 0 |
| Lmo0381 | 2 | 0 | 0 | 0 | 2 | 0 | 0 | 0 | 0 |
| Lmo0409 | 0 | 0 | 0 | 0 | 0 | 0 | 0 | 0 | 0 |
| Lmo0419 | 0 | 0 | 0 | 0 | 0 | 0 | 0 | 0 | 0 |
| Lmo0440 | 0 | 0 | 0 | 0 | 0 | 0 | 0 | 0 | 0 |
| Lmo0444 | 0 | 0 | 0 | 0 | 0 | 0 | 0 | 0 | 0 |
| Lmo0448 | 2 | 2 | 0 | 0 | 0 | 2 | 2 | 0 | 0 |
| Lmo0459 | 0 | 0 | 0 | 0 | 0 | 0 | 0 | 0 | 0 |
| Lmo0462 | 0 | 0 | 0 | 0 | 0 | 0 | 0 | 0 | 0 |
| Lmo0466 | 0 | 0 | 0 | 0 | 0 | 0 | 0 | 0 | 0 |
| Lmo0467 | 0 | 0 | 0 | 0 | 0 | 0 | 0 | 0 | 0 |
| Lmo0471 | 0 | 0 | 0 | 0 | 0 | 0 | 0 | 0 | 0 |
| Lmo0472 | 2 | 2 | 0 | 2 | 2 | 2 | 2 | 0 | 0 |
| Lmo0473 | 0 | 2 | 0 | 0 | 0 | 2 | 2 | 0 | 0 |
| Lmo0474 | 0 | 0 | 0 | 0 | 0 | 2 | 2 | 0 | 0 |
| Lmo0493 | 0 | 0 | 0 | 0 | 0 | 0 | 0 | 0 | 0 |
| Lmo0497 | 0 | 0 | 0 | 0 | 0 | 0 | 0 | 0 | 0 |
| Lmo0525 | 0 | 0 | 0 | 0 | 0 | 0 | 0 | 0 | 0 |
| Lmo0549 | 0 | 0 | 0 | 0 | 0 | 0 | 0 | 0 | 0 |
| Lmo0630 | 0 | 0 | 0 | 0 | 0 | 0 | 0 | 0 | 0 |
| Lmo0631 | 0 | 0 | 0 | 0 | 0 | 0 | 0 | 0 | 0 |
| Lmo0632 | 0 | 0 | 0 | 0 | 0 | 0 | 0 | 0 | 0 |
| Lmo0732 | 2 | 2 | 2 | 0 | 0 | 2 | 2 | 0 | 0 |
| Lmo0733 | 0 | 0 | 0 | 0 | 0 | 0 | 0 | 0 | 0 |
| Lmo0734 | 0 | 0 | 0 | 0 | 0 | 0 | 0 | 0 | 0 |
| Lmo0735 | 0 | 0 | 0 | 0 | 0 | 0 | 0 | 0 | 0 |
| Lmo0739 | 0 | 0 | 0 | 0 | 0 | 0 | 0 | 0 | 0 |
| Lmo0750 | 0 | 0 | 0 | 0 | 0 | 0 | 0 | 0 | 0 |
| Lmo0751 | 0 | 0 | 0 | 0 | 0 | 0 | 0 | 0 | 0 |
| Lmo0752 | 0 | 0 | 0 | 0 | 0 | 0 | 0 | 0 | 0 |
| Lmo0753 | 0 | 0 | 0 | 0 | 0 | 0 | 0 | 0 | 0 |
| Lmo0780 | 0 | 0 | 0 | 0 | 0 | 0 | 0 | 0 | 0 |
| Lmo0801 | 0 | 0 | 0 | 0 | 0 | 0 | 0 | 0 | 0 |
| Lmo0804 | 0 | 0 | 0 | 0 | 0 | 0 | 0 | 0 | 0 |
| Lmo0832 | 0 | 0 | 0 | 0 | 0 | 0 | 0 | 0 | 0 |
| Lmo0833 | 0 | 0 | 0 | 0 | 0 | 0 | 0 | 0 | 0 |
| Lmo0834 | 0 | 0 | 0 | 0 | 0 | 0 | 0 | 0 | 0 |
| Lmo0835 | 0 | 0 | 0 | 0 | 0 | 0 | 0 | 0 | 0 |
| Lmo0842 | 2 | 2 | 0 | 0 | 2 | 2 | 2 | 0 | 0 |
| Lmo1030 | 0 | 0 | 0 | 0 | 0 | 0 | 0 | 0 | 0 |
| Lmo1036 | 0 | 0 | 0 | 0 | 0 | 0 | 0 | 0 | 0 |
| Lmo1060 | 0 | 0 | 0 | 0 | 0 | 0 | 0 | 0 | 0 |
| Lmo1061 | 0 | 0 | 0 | 0 | 0 | 0 | 0 | 0 | 0 |
| Lmo1063 | 0 | 0 | 0 | 0 | 0 | 0 | 0 | 0 | 0 |
| Lmo1076 | 0 | 0 | 0 | 0 | 0 | 0 | 0 | 0 | 0 |
| Lmo1077 | 0 | 0 | 0 | 0 | 0 | 0 | 0 | 2 | 2 |
| Lmo1080 | 0 | 0 | 0 | 0 | 0 | 0 | 0 | 0 | 0 |
| Lmo1081 | 0 | 0 | 0 | 0 | 0 | 0 | 0 | 0 | 0 |
| Lmo1082 | 0 | 0 | 0 | 0 | 0 | 0 | 0 | 0 | 0 |
| Lmo1085 | 0 | 0 | 0 | 0 | 0 | 0 | 0 | 0 | 0 |
| Lmo1088 | 0 | 0 | 0 | 0 | 0 | 0 | 0 | 0 | 0 |
| Lmo1090 | 0 | 0 | 0 | 0 | 0 | 0 | 0 | 0 | 0 |
| Lmo1091 | 0 | 0 | 0 | 0 | 0 | 0 | 0 | 0 | 0 |
| Lmo1102 | 0 | 0 | 0 | 0 | 0 | 0 | 2 | 0 | 0 |
| Lmo1115 | 0 | 0 | 0 | 0 | 0 | 0 | 2 | 0 | 0 |
| Lmo1116 | 2 | 2 | 0 | 0 | 2 | 2 | 2 | 0 | 0 |
| Lmo1118 | 0 | 0 | 0 | 0 | 0 | 0 | 0 | 0 | 0 |
| Lmo1119 | 0 | 0 | 0 | 0 | 0 | 0 | 0 | 0 | 0 |
| Lmo1120 | 0 | 0 | 2 | 0 | 0 | 0 | 0 | 0 | 0 |
| Lmo1133 | 0 | 0 | 0 | 0 | 0 | 0 | 0 | 0 | 0 |
| Lmo1135 | 0 | 0 | 0 | 0 | 0 | 0 | 0 | 0 | 0 |
| Lmo1307 | 0 | 0 | 0 | 0 | 0 | 0 | 0 | 0 | 0 |
| Lmo1441 | 0 | 0 | 0 | 0 | 0 | 0 | 0 | 0 | 0 |
| Lmo1451 | 0 | 0 | 0 | 0 | 0 | 0 | 0 | 0 | 0 |
| Lmo1597 | 0 | 0 | 0 | 0 | 0 | 0 | 0 | 0 | 0 |
| Lmo1604 | 2 | 2 | 2 | 0 | 0 | 2 | 2 | 0 | 0 |
| Lmo1648 | 2 | 0 | 0 | 0 | 0 | 0 | 2 | 0 | 0 |
| Lmo1666 | 0 | 0 | 0 | 0 | 0 | 0 | 0 | 0 | 0 |
| Lmo1714 | 0 | 0 | 0 | 0 | 0 | 2 | 2 | 0 | 0 |
| Lmo1968 | 0 | 2 | 0 | 0 | 0 | 0 | 0 | 0 | 0 |
| Lmo1973 | 0 | 2 | 0 | 0 | 0 | 0 | 0 | 0 | 0 |
| Lmo1974 | 0 | 2 | 0 | 0 | 0 | 0 | 0 | 0 | 0 |
| Lmo2026 | 0 | 0 | 0 | 0 | 0 | 0 | 0 | 0 | 0 |
| Lmo2027 | 0 | 0 | 0 | 0 | 0 | 0 | 0 | 0 | 0 |
| Lmo2276 | 0 | 0 | 0 | 0 | 0 | 0 | 0 | 0 | 0 |
| Lmo2277 | 2 | 0 | 0 | 0 | 0 | 0 | 0 | 0 | 0 |
| Lmo2311 | 0 | 0 | 0 | 0 | 0 | 0 | 0 | 0 | 0 |
| Lmo2312 | 2 | 0 | 0 | 0 | 0 | 2 | 0 | 0 | 0 |
| Lmo2324 | 2 | 2 | 0 | 0 | 2 | 0 | 0 | 0 | 0 |
| Lmo2408 | 0 | 0 | 0 | 0 | 0 | 0 | 0 | 0 | 0 |
| Lmo2443 | 0 | 0 | 0 | 0 | 0 | 0 | 0 | 0 | 0 |
| Lmo2576 | 0 | 0 | 0 | 0 | 0 | 0 | 0 | 0 | 0 |
| Lmo2594 | 2 | 2 | 0 | 0 | 0 | 2 | 0 | 0 | 0 |
| Lmo2595 | 0 | 0 | 0 | 0 | 0 | 0 | 0 | 0 | 0 |
| Lmo2671 | 0 | 0 | 0 | 0 | 0 | 0 | 0 | 0 | 0 |
| Lmo2686 | 0 | 0 | 0 | 0 | 0 | 0 | 0 | 0 | 0 |
| Lmo2732 | 0 | 0 | 0 | 0 | 0 | 0 | 0 | 0 | 0 |
| Lmo2736 | 0 | 0 | 0 | 0 | 0 | 0 | 0 | 0 | 0 |
| Lmo2786 | 0 | 0 | 0 | 0 | 0 | 0 | 0 | 0 | 0 |
| Lmo2787 | 0 | 0 | 0 | 0 | 0 | 0 | 0 | 0 | 0 |
| Lmo2807 | 0 | 0 | 0 | 0 | 0 | 0 | 0 | 0 | 0 |
| Lmo2809 | 0 | 0 | 0 | 0 | 0 | 2 | 2 | 0 | 0 |
| Lmo2821 | 0 | 0 | 0 | 0 | 0 | 0 | 0 | 0 | 0 |
| Lin0060 | 0 | 0 | 2 | 2 | 0 | 2 | 2 | 0 | 0 |
| Lin0197 | 2 | 2 | 2 | 2 | 2 | 2 | 2 | 0 | 0 |
| Lin0332 | 0 | 0 | 2 | 2 | 0 | 0 | 0 | 0 | 0 |
| Lin0397 | 0 | 2 | 2 | 2 | 0 | 0 | 0 | 0 | 0 |
| Lin0415 | 2 | 2 | 2 | 2 | 2 | 2 | 2 | 0 | 0 |
| Lin0453 | 2 | 2 | 2 | 2 | 2 | 2 | 2 | 0 | 0 |
| Lin0454 | 2 | 2 | 2 | 2 | 2 | 2 | 2 | 0 | 0 |
| Lin0455 | 2 | 2 | 2 | 2 | 2 | 2 | 2 | 0 | 0 |
| Lin0465 | 2 | 2 | 2 | 2 | 2 | 2 | 2 | 0 | 0 |
| Lin0476 | 0 | 2 | 2 | 0 | 0 | 0 | 0 | 0 | 0 |
| Lin0661 | 2 | 2 | 2 | 2 | 2 | 2 | 2 | 0 | 0 |
| Lin0665 | 2 | 2 | 2 | 2 | 2 | 2 | 2 | 0 | 0 |
| Lin0677 | 2 | 2 | 2 | 2 | 2 | 2 | 2 | 0 | 0 |
| Lin0739 | 2 | 2 | 2 | 2 | 2 | 2 | 2 | 0 | 0 |
| Lin0740 | 2 | 2 | 2 | 2 | 2 | 2 | 2 | 0 | 0 |
| Lin0803 | 2 | 2 | 2 | 2 | 2 | 2 | 2 | 0 | 0 |
| Lin0868 | 0 | 2 | 2 | 2 | 0 | 0 | 0 | 0 | 0 |
| Lin1066 | 0 | 2 | 2 | 2 | 0 | 0 | 0 | 0 | 0 |
| Lin1069 | 0 | 2 | 2 | 2 | 2 | 0 | 0 | 0 | 0 |
| Lin1073 | 2 | 2 | 2 | 2 | 2 | 2 | 2 | 0 | 0 |
| Lin1083 | 0 | 0 | 2 | 2 | 0 | 0 | 0 | 0 | 0 |
| Lin1084 | 2 | 2 | 2 | 2 | 0 | 2 | 2 | 0 | 0 |
| Lin1090 | 0 | 2 | 2 | 2 | 0 | 2 | 2 | 0 | 0 |
| Lin1204 | 0 | 0 | 2 | 2 | 0 | 2 | 2 | 0 | 0 |
| Lin1262 | 2 | 2 | 2 | 0 | 0 | 2 | 2 | 0 | 0 |
| Lin1299 | 2 | 2 | 2 | 2 | 2 | 2 | 2 | 0 | 0 |
| Lin1328 | 2 | 2 | 2 | 2 | 2 | 2 | 2 | 0 | 0 |
| Lin1452 | 2 | 2 | 2 | 2 | 2 | 2 | 2 | 0 | 0 |
| Lin1621 | 2 | 0 | 2 | 2 | 2 | 2 | 2 | 0 | 0 |
| Lin2397 | 2 | 2 | 2 | 0 | 0 | 2 | 2 | 0 | 0 |
| Lin2494 | 2 | 2 | 2 | 2 | 2 | 2 | 2 | 0 | 0 |
| Lin2603 | 0 | 0 | 2 | 0 | 2 | 2 | 2 | 0 | 0 |
| Lin2724 | 2 | 2 | 2 | 2 | 2 | 2 | 2 | 0 | 0 |
| Lin2741 | 2 | 2 | 2 | 2 | 2 | 2 | 2 | 0 | 0 |
| Lin2743 | 2 | 0 | 2 | 2 | 2 | 2 | 2 | 0 | 0 |
| Lin2744 | 2 | 2 | 2 | 2 | 2 | 2 | 2 | 0 | 0 |
| Lin2839 | 2 | 2 | 2 | 2 | 2 | 2 | 2 | 0 | 0 |
| Lin2918 | 2 | 2 | 2 | 2 | 2 | 2 | 2 | 0 | 0 |
| ORF 105 | 0 | 0 | 0 | 0 | 0 | 2 | 2 | 0 | 0 |
| ORF 1390 | 2 | 0 | 0 | 0 | 0 | 0 | 2 | 0 | 0 |
| ORF 1445 | 0 | 0 | 0 | 0 | 0 | 0 | 0 | 0 | 0 |
| ORF 1490 | 0 | 0 | 0 | 0 | 0 | 0 | 0 | 0 | 0 |
| ORF 1491 | 0 | 0 | 0 | 0 | 0 | 0 | 0 | 0 | 0 |
| ORF 1590 | 0 | 0 | 0 | 0 | 0 | 0 | 0 | 0 | 0 |
| ORF 1761 | 0 | 0 | 0 | 0 | 0 | 0 | 0 | 0 | 0 |
| ORF 2017 | 2 | 0 | 0 | 0 | 0 | 2 | 0 | 0 | 0 |
| ORF 2074 | 2 | 0 | 0 | 0 | 2 | 2 | 2 | 0 | 0 |
| ORF 2076 | 2 | 0 | 0 | 0 | 2 | 2 | 2 | 0 | 0 |
| ORF 2110 | 0 | 0 | 0 | 0 | 0 | 0 | 0 | 0 | 0 |
| ORF 2372 | 0 | 0 | 0 | 0 | 0 | 0 | 0 | 0 | 0 |
| ORF 2541 | 0 | 0 | 0 | 0 | 0 | 0 | 0 | 0 | 0 |
| ORF 2579 | 2 | 0 | 0 | 0 | 2 | 2 | 0 | 0 | 0 |
| ORF 2777 | 0 | 0 | 0 | 0 | 0 | 0 | 0 | 0 | 0 |
| ORF 2819 | 0 | 0 | 0 | 0 | 0 | 0 | 0 | 0 | 0 |
| ORF 2847 | 0 | 0 | 0 | 0 | 0 | 0 | 0 | 0 | 0 |
| ORF 2858 | 2 | 2 | 0 | 0 | 0 | 2 | 0 | 2 | 2 |
| ORF 3103 | 2 | 0 | 0 | 0 | 2 | 0 | 0 | 0 | 0 |
| ORF 3184 | 2 | 0 | 0 | 0 | 2 | 0 | 0 | 0 | 0 |
| ORF 3401 | 2 | 0 | 0 | 0 | 0 | 2 | 0 | 0 | 0 |
| ORF 3792 | 0 | 0 | 0 | 0 | 0 | 0 | 0 | 0 | 0 |
| ORF 3840 | 0 | 0 | 0 | 0 | 0 | 0 | 0 | 0 | 0 |
| ORF 4101 | 2 | 2 | 0 | 0 | 2 | 2 | 2 | 0 | 0 |
| ORF 660 | 0 | 0 | 0 | 0 | 0 | 0 | 0 | 0 | 0 |
| ORF 757 | 2 | 2 | 0 | 0 | 2 | 0 | 0 | 0 | 0 |
| ORF 799 | 2 | 2 | 0 | 0 | 2 | 2 | 2 | 0 | 0 |
| ORF 817 | 0 | 2 | 0 | 0 | 0 | 0 | 0 | 0 | 0 |

TABLE 17

| L. monocytogenes | | | | | | | | | | | | |
|---|---|---|---|---|---|---|---|---|---|---|---|---|
| | | | | | Lineage | Lineage | Lineage | Lineage | | | | |
| | | | | | I | I | II | II | Lineage | Lineage | | |
| | | | | | I.1 | I.2 | II.1 | II.2 | III | III | | |
| | | | | L. | 1/2a- | 1/2c- | 1/2b- | 4b- | III.1 | III.2 | | |
| L. | L. | L. | L. | grayi- | 3a | 3c | 3b-7 | 4d-4e | 4a | 4c | | |
| ivanovii | welshimeri | seeligeri | innocua | murray | Strains | strains | strains | strains | strains | strains | Gene description | Gene function category |

a: Percentage of presence of a specific gene in the different Listeria species and L. monocytogenes sub-groups
L. monocytogenes EGDe genes

| | | | | | | | | | | | | | |
|---|---|---|---|---|---|---|---|---|---|---|---|---|---|
| Lmo0082 | 0 | 0 | 0 | 0 | 0 | 100 | 100 | 100 | 100 | 100 | 100 | 108aa/Unknwon | unknown |
| Lmo0083 | 0 | 0 | 0 | 0 | 0 | 100 | 100 | 100 | 100 | 100 | 100 | 122aa/unknown, similar to transcription | Regulation |
| | | | | | | | | | | | | regulator (merR family) | |
| Lmo0200 | 50 | 0 | 50 | 25 | 100 | 100 | 100 | 100 | 100 | 100 | 100 | 237aa/prfA: listeriolysin positive | Regulation |
| | | | | | | | | | | | | regulatory protein | |
| Lmo0201 | 0 | 0 | 0 | 0 | 0 | 100 | 100 | 100 | 100 | 100 | 100 | 317aa/plcA: phosphatidylinositol-specific | virulence |
| | | | | | | | | | | | | phospholipase c | |
| Lmo0202 | 83 | 0 | 50 | 0 | 0 | 100 | 100 | 100 | 100 | 100 | 100 | 529aa/hly | virulence |
| Lmo0203 | 50 | 0 | 0 | 0 | 0 | 100 | 100 | 100 | 100 | 100 | 100 | 510aa/mpl: Zinc matalloproteinase | virulence |
| | | | | | | | | | | | | precursor | |
| Lmo0204 | 0 | 0 | 0 | 0 | 0 | 100 | 100 | 100 | 100 | 100 | 100 | 639aa/actA: actin-assembly inducing | virulence |
| | | | | | | | | | | | | protein precursor | |
| Lmo0205 | 0 | 0 | 0 | 0 | 0 | 100 | 100 | 100 | 100 | 100 | 100 | 289aa/plcB: phospholipase C | virulence |
| Lmo0206 | 0 | 0 | 0 | 0 | 0 | 100 | 100 | 100 | 100 | 100 | 100 | 107aa/unknwon | unknown |
| Lmo0433 | 0 | 0 | 0 | 0 | 0 | 100 | 100 | 100 | 100 | 100 | 100 | 800aa/InlA: Internalin A | cell surface proteins |
| Lmo0434 | 0 | 0 | 0 | 0 | 0 | 100 | 100 | 100 | 100 | 100 | 100 | 630aa/InlB: Internalin B | cell surface proteins |
| Lmo0440 | 0 | 0 | 0 | 0 | 0 | 100 | 100 | 100 | 100 | 100 | 100 | 167aa/unknown | unknown |
| Lmo0550 | 0 | 0 | 0 | 13 | 0 | 100 | 100 | 100 | 100 | 100 | 100 | 98aa/unknown, pepdidoglycan bound | cell surface proteins |
| | | | | | | | | | | | | protein (LPXTG motif) | |
| Lmo0733 | 0 | 0 | 0 | 0 | 0 | 100 | 100 | 100 | 100 | 100 | 100 | 169aa/unknown, similar to transcription | Regulation |
| | | | | | | | | | | | | regulator | |
| Lmo1133 | 0 | 0 | 0 | 0 | 0 | 100 | 100 | 100 | 100 | 100 | 100 | 102aa/unknown, similar to B. subtilis YjcS | unknown |
| | | | | | | | | | | | | protein | |
| Lmo2732 | 0 | 0 | 0 | 0 | 0 | 100 | 100 | 100 | 100 | 100 | 100 | 250aa/unknwon | unknown |
| Lmo2733 | 0 | 0 | 0 | 0 | 0 | 100 | 100 | 100 | 100 | 100 | 100 | 652aa/unknown, similar to PTS system, | Transport/binding proteins |
| | | | | | | | | | | | | fructose-specific IIABC component | and lipoproteins |
| Lmo2734 | 0 | 0 | 0 | 0 | 0 | 100 | 100 | 100 | 100 | 100 | 100 | 882aa/unknown, weakly similar to sugar | Specific pathways |
| | | | | | | | | | | | | hydrolase | |
| Lmo2736 | 0 | 0 | 0 | 0 | 0 | 100 | 100 | 100 | 100 | 100 | 100 | 377aa/unknown, conserved hypothetical | unknown |
| | | | | | | | | | | | | protein | |
| Lmo0160 | 0 | 0 | 0 | 13 | 0 | 100 | 100 | 100 | 96 | 100 | 100 | 571aa/unknown, putative peptidoglycan | Cell surface proteins |
| | | | | | | | | | | | | bound protein (LPXTG motif) | |
| Lmo0833 | 0 | 0 | 0 | 0 | 0 | 100 | 100 | 100 | 100 | 33 | 100 | 296aa/unknown; similar to transcriptional | Regulation |
| | | | | | | | | | | | | regulator | |
| Lmo1135 | 0 | 0 | 0 | 0 | 0 | 100 | 100 | 100 | 100 | 33 | 100 | 383aa/unknwon | unknown |
| Lmo0017 | 0 | 0 | 0 | 0 | 0 | 100 | 100 | 100 | 100 | 0 | 100 | 372aa/unknown, similar to Bacillus | Cell wall |
| | | | | | | | | | | | | anthracis CapA protein (polyglutamate | |
| | | | | | | | | | | | | capsule biosynthesis) | |
| Lmo0263 | 0 | 0 | 0 | 0 | 0 | 100 | 100 | 100 | 100 | 0 | 100 | 548aa/InlH: Internalin H | cell surface proteins |
| Lmo0264 | 0 | 0 | 0 | 0 | 0 | 100 | 100 | 100 | 100 | 0 | 100 | 499aa/InlE: Internalin E | cell surface proteins |
| Lmo0333 | 0 | 0 | 0 | 0 | 0 | 100 | 100 | 100 | 100 | 0 | 100 | 1778aa/unknown, similar to internalin | cell surface proteins |
| | | | | | | | | | | | | proteins, putative peptidoglycan bound | |
| | | | | | | | | | | | | protein (LPXTG motif) | |
| Lmo0334 | 0 | 50 | 0 | 0 | 0 | 100 | 100 | 100 | 100 | 0 | 100 | 174aa/unknwon | unknown |
| Lmo0419 | 0 | 100 | 0 | 0 | 0 | 100 | 100 | 100 | 100 | 0 | 100 | 184aa/unknown, similar to unknown | unknown |
| | | | | | | | | | | | | protein | |
| Lmo0549 | 0 | 0 | 0 | 0 | 0 | 100 | 100 | 100 | 100 | 0 | 100 | 673aa/unknown, similar to internalin | cell surface proteins |
| | | | | | | | | | | | | protein | |
| Lmo0834 | 0 | 0 | 0 | 0 | 0 | 100 | 100 | 100 | 100 | 0 | 100 | 237aa/Unknwon | unknown |
| Lmo2821 | 0 | 0 | 0 | 0 | 0 | 100 | 100 | 100 | 100 | 0 | 100 | 851aa/unknown, similar to internalin, | Cell surface proteins |
| | | | | | | | | | | | | unknown, putative peptidoglycen bound | |
| | | | | | | | | | | | | protein (LPXTG motif) | |
| Lmo1786 | 100 | 0 | 0 | 0 | 0 | 100 | 100 | 100 | 100 | 0 | 50 | 296aa/InlC | cell surface proteins |
| Lmo0094 | 0 | 0 | 0 | 0 | 0 | 100 | 100 | 100 | 100 | 0 | 0 | 186aa/unknwon | unknown |
| Lmo0832 | 0 | 0 | 0 | 0 | 0 | 100 | 100 | 100 | 100 | 0 | 0 | 94aa/unknown, similar to transposase | Transposon and IS |
| Lmo0835 | 0 | 0 | 0 | 0 | 0 | 100 | 100 | 100 | 100 | 0 | 0 | 334aa/unknown, putative peptidoglycan | cell surface proteins |
| | | | | | | | | | | | | bound protein (LPXTG motif) | |
| Lmo1441 | 0 | 0 | 0 | 0 | 0 | 100 | 100 | 100 | 100 | 0 | 0 | 368aa/unknown, similar to putative | Cell wall |
| | | | | | | | | | | | | peptidoglycan acetylation protein | |
| Lmo1451 | 0 | 0 | 0 | 0 | 0 | 100 | 100 | 100 | 100 | 0 | 0 | 331aa/unknown, similar to E. coli LytB | unknown |
| | | | | | | | | | | | | protein | |
| Lmo1666 | 0 | 0 | 50 | 0 | 0 | 100 | 100 | 100 | 100 | 0 | 0 | 1711aa/unknown, peptidoglycan linked | cell surface proteins |
| | | | | | | | | | | | | protein (LPxTG) | |
| Lmo0933 | 67 | 100 | 100 | 50 | 0 | 100 | 100 | 100 | 0 | 0 | 0 | 315aa/unknown, similar to sugar | Cell wall |
| | | | | | | | | | | | | transferase | |
| Lmo1090 | 0 | 0 | 100 | 0 | 0 | 100 | 100 | 100 | 0 | 0 | 50 | 327aa/unknown, similar to | Cell wall |
| | | | | | | | | | | | | glycosyltransferases | |
| Lmo0497 | 0 | 0 | 0 | 0 | 0 | 100 | 100 | 100 | 4 | 0 | 0 | 502aa/unknown, similar to sugar | Cell wall |
| | | | | | | | | | | | | transferase | |
| Lmo1076 | 0 | 0 | 100 | 0 | 0 | 100 | 100 | 100 | 0 | 0 | 0 | 572aa/unknown, similar to AUTOLYSIN | Cell wall |
| | | | | | | | | | | | | (EC 3.5.1.28) (N-ACETYLMURAMOYL-L- | |
| | | | | | | | | | | | | ALANINE AMIDASE) | |
| Lmo1077 | 0 | 0 | 100 | 0 | 100 | 100 | 100 | 100 | 0 | 0 | 0 | 562aa/unknown, similar to teichoic acid | Cell wall |
| | | | | | | | | | | | | biosynthesis protein B | |
| Lmo1079 | 0 | 0 | 100 | 0 | 0 | 100 | 100 | 100 | 0 | 0 | 0 | 952aa/unknown, similar to B. subtilis YfhO | unknown |
| | | | | | | | | | | | | protein | |
| Lmo1080 | 0 | 0 | 50 | 0 | 0 | 100 | 100 | 100 | 0 | 0 | 0 | 623aa/unknown, similar to B. subtilis | Cell wall |
| | | | | | | | | | | | | minor teichoic acids biosynthesis protein | |
| | | | | | | | | | | | | GgaB | |
| Lmo1081 | 0 | 0 | 100 | 0 | 0 | 100 | 100 | 100 | 0 | 0 | 0 | 288aa/unknown, similar to glucose-1- | Cell wall |
| | | | | | | | | | | | | phosphate thymidyl transferase | |
| Lmo1082 | 0 | 0 | 100 | 0 | 0 | 100 | 100 | 100 | 0 | 0 | 0 | 186aa/unknown, similar to dTDP-sugar | Cell wall |
| | | | | | | | | | | | | epimerase | |
| Lmo1083 | 0 | 0 | 100 | 0 | 0 | 100 | 100 | 100 | 0 | 0 | 0 | 328aa/unknown, similar to dTDP-D- | Cell wall |
| | | | | | | | | | | | | glucose 4,6-dehydratase | |
| Lmo1084 | 0 | 0 | 100 | 0 | 0 | 100 | 100 | 100 | 0 | 0 | 0 | 276aa/unknown, similar to DTDP-L- | Cell wall |
| | | | | | | | | | | | | rhamnose synthetase | |
| Lmo1085 | 0 | 0 | 0 | 0 | 0 | 100 | 100 | 100 | 0 | 0 | 0 | 696aa/unknown, similar to teichoic acid | Cell wall |
| | | | | | | | | | | | | biosynthesis protein B | |
| Lmo1088 | 0 | 0 | 50 | 0 | 0 | 100 | 100 | 100 | 0 | 0 | 0 | 386aa/tagB: unknown, similar to teichoic | Cell wall |
| | | | | | | | | | | | | acid biosynthesis protein B precursor | |
| Lmo1091 | 0 | 0 | 100 | 0 | 0 | 100 | 100 | 100 | 0 | 0 | 0 | 337aa/unknown, slumilar to | Cell wall |
| | | | | | | | | | | | | glysosyltransferases | |
| Lmo0171 | 0 | 0 | 0 | 0 | 0 | 100 | 100 | 0 | 0 | 0 | 0 | 832aa/unknwon, similar to internalin | cell surface proteins |
| | | | | | | | | | | | | proteins, putative peptidoglycan bound | |
| | | | | | | | | | | | | protein (LPXTG motif) | |
| Lmo0172 | 0 | 0 | 0 | 0 | 0 | 100 | 100 | 0 | 0 | 0 | 0 | 112aa/unknown, similar to transposase C- | Transposon and IS |
| | | | | | | | | | | | | terminal part | |
| Lmo0525 | 0 | 50 | 0 | 0 | 0 | 100 | 100 | 0 | 0 | 0 | 0 | 443aa/unknwon | unknown |
| Lmo0734 | 0 | 100 | 0 | 0 | 0 | 100 | 100 | 0 | 0 | 0 | 0 | 334aa/unknown, similar to transcriptional | Regulation |
| | | | | | | | | | | | | regulator (Lacl family) | |
| Lmo0735 | 17 | 50 | 0 | 0 | 0 | 100 | 100 | 0 | 0 | 0 | 0 | 223aa/unknown, similar to Ribulose-5- | Specific pathways |
| | | | | | | | | | | | | Phosphate 3-Epimerase | |
| Lmo0736 | 67 | 100 | 0 | 0 | 0 | 100 | 100 | 0 | 0 | 0 | 0 | 148aa/unknown, similar to ribose 5- | Specific pathways |
| | | | | | | | | | | | | phosphate isomerase | |
| Lmo0737 | 0 | 0 | 0 | 0 | 0 | 100 | 100 | 0 | 0 | 0 | 0 | 310aa/Unknwon | unknown |
| Lmo0738 | 33 | 0 | 0 | 0 | 0 | 100 | 100 | 0 | 0 | 0 | 0 | 617aa/unknown, similar to | Transport/binding proteins |
| | | | | | | | | | | | | phosphotransferase system (PTS) beta- | and lipoproteins |
| | | | | | | | | | | | | glucoside-specific enzyme IIABC | |
| | | | | | | | | | | | | component | |
| Lmo0739 | 0 | 0 | 0 | 0 | 0 | 100 | 100 | 0 | 0 | 0 | 0 | 457aa/unknown, similar to 6-phospho-beta- | Specific pathways |
| | | | | | | | | | | | | glucosidase | |
| Lmo1060 | 0 | 100 | 0 | 0 | 0 | 100 | 100 | 0 | 0 | 0 | 0 | 220aa/unknown, similar to transcription | Regulation |
| | | | | | | | | | | | | response regulator | |
| Lmo1061 | 0 | 50 | 0 | 0 | 0 | 100 | 100 | 0 | 0 | 0 | 0 | 481aa/unknown, similar to sensor protein | Sensors (signal transduction) |
| | | | | | | | | | | | | histidine kinases (2 components | |
| | | | | | | | | | | | | regulatory systems) | |
| Lmo1062 | 0 | 100 | 0 | 0 | 0 | 100 | 100 | 0 | 0 | 0 | 0 | 473aa/unknown, unknown, similar to ABC | Transport/binding proteins |
| | | | | | | | | | | | | transporters (permease protein) | and lipoproteins |
| Lmo1063 | 0 | 100 | 0 | 0 | 0 | 100 | 100 | 0 | 0 | 0 | 0 | 222aa/unknown, similar to ABC | Transport/binding proteins |
| | | | | | | | | | | | | transporter (ATP binding protein) | and lipoproteins |
| Lmo1968 | 0 | 0 | 0 | 13 | 0 | 100 | 100 | 0 | 0 | 0 | 0 | 247aa/unknown, similar to creatinine | Metabolism of amino acids |
| | | | | | | | | | | | | amidohydrolases | and related molecules |
| Lmo1969 | 0 | 0 | 0 | 13 | 0 | 100 | 100 | 0 | 0 | 0 | 0 | 202aa/unknown, similar to 2-keto-3- | Specific pathways |
| | | | | | | | | | | | | deoxygluconate-6-phosphate aldoiase | |
| Lmo1970 | 0 | 0 | 0 | 13 | 0 | 100 | 100 | 0 | 0 | 0 | 0 | 305aa/unknown, similar to putative | Metabolism of lipids |
| | | | | | | | | | | | | phosphotriesterase related proteins | |
| Lmo1971 | 0 | 0 | 0 | 13 | 0 | 100 | 100 | 0 | 0 | 0 | 0 | 422aa/unknown, similar to pentitol PTS | Transport/binding proteins |
| | | | | | | | | | | | | system enzyme II C component | and lipoproteins |
| Lmo1973 | 0 | 0 | 0 | 13 | 0 | 100 | 100 | 0 | 0 | 0 | 0 | 147aa/unknown, similar to PTS system | Transport/binding proteins |
| | | | | | | | | | | | | enzyme II A component | and lipoproteins |
| Lmo1974 | 0 | 0 | 0 | 13 | 0 | 100 | 100 | 0 | 0 | 0 | 0 | 249aa/unknown, similar to transcription | Regulation |
| | | | | | | | | | | | | regulators, (GntR family) | |
| Lmo0151 | 0 | 0 | 0 | 0 | 0 | 11 | 100 | 0 | 0 | 0 | 0 | 135aa/unknwon | unknown |
| Lmo0466 | 0 | 0 | 0 | 0 | 0 | 11 | 100 | 0 | 0 | 0 | 0 | 567aa/unknown | unknown |
| Lmo0467 | 0 | 0 | 0 | 0 | 0 | 11 | 100 | 0 | 0 | 0 | 0 | 107aa/unknwon | unknown |
| Lmo0469 | 0 | 0 | 0 | 0 | 0 | 11 | 100 | 0 | 0 | 0 | 0 | 290aa/unknwon | unknown |
| Lmo0470 | 0 | 0 | 0 | 0 | 0 | 11 | 100 | 0 | 0 | 0 | 0 | 209aa/unknown, weakly similar to site- | DNA restriction/modification |
| | | | | | | | | | | | | specific DNA-methyltransferase | and repair |
| Lmo0471 | 0 | 0 | 0 | 0 | 0 | 11 | 100 | 0 | 0 | 0 | 0 | 336aa/unknwon | unknown |
| Lmo1118 | 17 | 0 | 0 | 0 | 0 | 4 | 100 | 0 | 0 | 0 | 0 | 505aa/unknwon | unknown |
| Lmo1119 | 0 | 0 | 0 | 0 | 0 | 4 | 100 | 0 | 0 | 0 | 0 | 285aa/unknown, similar to methylases | DNA restriction/modification |
| | | | | | | | | | | | | | and repair |
| Lmo0752 | 100 | 0 | 0 | 0 | 0 | 100 | 100 | 100 | 100 | 100 | 0 | 278aa/unknown, weakly similar to a | Detoxification |
| | | | | | | | | | | | | putative haloacetate dehalogenase | |
| Lmo0753 | 100 | 0 | 0 | 0 | 0 | 100 | 100 | 100 | 100 | 100 | 0 | 226aa/unknown, similar to transcription | Regulation |
| | | | | | | | | | | | | regulator Crp/Fnr family | |
| Lmo0754 | 100 | 0 | 0 | 0 | 0 | 100 | 100 | 100 | 100 | 100 | 0 | 203aa/unknown, weakly similar to a bile | Detoxification |
| | | | | | | | | | | | | acid 7-alpha dehydratase | |
| Lmo1030 | 100 | 0 | 0 | 0 | 0 | 100 | 100 | 100 | 100 | 0 | 100 | 342aa/unknown, similar to transcriptional | Regulation |
| | | | | | | | | | | | | regulator, Lacl family | |
| Lmo1031 | 100 | 0 | 0 | 0 | 0 | 100 | 100 | 100 | 100 | 0 | 100 | 467aa/unknown, similar to hypothetical | unknown |
| | | | | | | | | | | | | proteins | |
| Lmo1033 | 100 | 0 | 0 | 0 | 0 | 100 | 100 | 100 | 100 | 0 | 100 | 318aa/unknown, similar to transketolase | Specific pathways |
| Lmo1034 | 100 | 0 | 0 | 0 | 0 | 100 | 100 | 100 | 100 | 0 | 100 | 487aa/unknown, similar to glycerol kinase | Specific pathways |
| Lmo1035 | 100 | 0 | 0 | 0 | 0 | 100 | 100 | 100 | 100 | 0 | 100 | 618aa/unknown, similar to | Transport/binding proteins |
| | | | | | | | | | | | | phosphotransferase system (PTS) beta- | and lipoproteins |
| | | | | | | | | | | | | glucoside-specific enzyme IIABC | |
| Lmo1036 | 100 | 0 | 0 | 0 | 0 | 100 | 100 | 100 | 100 | 0 | 100 | 421aa/unknwon | unknown |
| Lmo2157 | 100 | 0 | 0 | 0 | 0 | 100 | 100 | 100 | 100 | 0 | 100 | 633aa/SepA-unknown | unknown |
| Lmo0066 | 50 | 0 | 0 | 25 | 0 | 22 | 100 | 15 | 7 | 33 | 100 | 604aa/unknwon, similar to toxin | miscellaneous |
| | | | | | | | | | | | | components | |
| Lmo0067 | 0 | 0 | 0 | 25 | 0 | 7 | 100 | 10 | 0 | 0 | 0 | 329aa/unknown, similar to dinitrogenase | Protein modification |
| | | | | | | | | | | | | reductase ADP-ribosylation system | |
| Lmo0068 | 0 | 0 | 0 | 25 | 0 | 7 | 100 | 45 | 59 | 0 | 0 | 107aa/unknwon | unknown |
| Lmo0069 | 0 | 0 | 0 | 0 | 0 | 19 | 100 | 80 | 59 | 0 | 0 | 97aa/unknwon | unknown |
| Lmo0071 | 0 | 0 | 0 | 0 | 0 | 19 | 100 | 95 | 59 | 0 | 0 | 85aa/Unknwon | unknown |
| Lmo0072 | 0 | 0 | 0 | 25 | 0 | 41 | 100 | 90 | 100 | 0 | 0 | 67aa/unknown, Hypothetical | unknown |
| Lmo0073 | 0 | 0 | 0 | 0 | 0 | 11 | 100 | 50 | 59 | 0 | 0 | 94aa/unknwon | unknown |
| Lmo0074 | 0 | 0 | 0 | 0 | 0 | 11 | 100 | 45 | 59 | 0 | 0 | 149aa/unknwon | unknown |
| Lmo0079 | 33 | 0 | 50 | 0 | 0 | 67 | 92 | 100 | 81 | 0 | 100 | 492aa/unknown | unknown |
| Lmo0080 | 0 | 0 | 50 | 0 | 0 | 48 | 100 | 95 | 85 | 0 | 0 | 132aa/unknwon | unknown |
| Lmo0081 | 0 | 0 | 0 | 0 | 0 | 15 | 92 | 35 | 0 | 0 | 0 | 200aa/unknwon | unknown |
| Lmo0141 | 0 | 0 | 0 | 0 | 0 | 30 | 100 | 25 | 96 | 100 | 50 | 80aa/unknwon | unknown |
| Lmo0143 | 0 | 0 | 0 | 0 | 0 | 22 | 100 | 5 | 7 | 100 | 0 | 58aa/unknwon | unknown |
| Lmo0144 | 0 | 0 | 0 | 0 | 0 | 11 | 100 | 25 | 96 | 100 | 50 | 147aa/unknwon | unknown |
| Lmo0146 | 0 | 0 | 0 | 25 | 0 | 33 | 100 | 35 | 96 | 100 | 100 | 68aa/unknwon, hypothetical protein | unknown |
| Lmo0252 | 0 | 100 | 50 | 0 | 0 | 44 | 100 | 0 | 0 | 100 | 100 | 128aa/unknown, similar to repressor | Regulation |
| | | | | | | | | | | | | (penicilinase repressor) | |
| Lmo0253 | 0 | 0 | 50 | 0 | 0 | 44 | 100 | 0 | 0 | 100 | 100 | 346aa/unknown, similar to penicillinase | Regulation |
| | | | | | | | | | | | | antirepressor | |
| Lmo0255 | 0 | 0 | 0 | 0 | 0 | 93 | 100 | 30 | 22 | 100 | 100 | 167aa/unknown, similar to unknown | unknown |
| | | | | | | | | | | | | protein | |
| Lmo0262 | 0 | 0 | 0 | 0 | 0 | 63 | 100 | 0 | 4 | 0 | 100 | 490aa/InIG | cell surface proteins |
| Lmo0304 | 0 | 50 | 0 | 50 | 0 | 56 | 100 | 90 | 7 | 0 | 50 | 421aa/Unknwon | unknown |
| Lmo0310 | 0 | 0 | 0 | 0 | 0 | 7 | 100 | 30 | 4 | 0 | 0 | 358aa/unknown | unknown |
| Lmo0311 | 0 | 0 | 0 | 0 | 0 | 7 | 100 | 30 | 4 | 0 | 0 | 206aa/unknwon | unknown |
| Lmo0312 | 0 | 0 | 0 | 0 | 0 | 7 | 100 | 30 | 4 | 0 | 0 | 400aa/unknown, similar to unknown | unknown |
| | | | | | | | | | | | | proteins | |
| Lmo0313 | 0 | 0 | 0 | 0 | 0 | 7 | 100 | 30 | 4 | 0 | 0 | 248aa/unknown, conserved hypothetical | unknown |
| | | | | | | | | | | | | protein | |
| Lmo0320 | 0 | 0 | 0 | 13 | 0 | 85 | 100 | 90 | 100 | 33 | 0 | 399aa/unknown, similar to surface protein | cell surface proteins |
| | | | | | | | | | | | | (peptidoglycan bound, LPXTG motif) | |
| Lmo0338 | 33 | 0 | 0 | 13 | 50 | 67 | 100 | 0 | 0 | 0 | 0 | 126aa/unknwon | unknown |
| Lmo0378 | 0 | 0 | 0 | 50 | 0 | 100 | 92 | 75 | 19 | 0 | 0 | 148aa/unknown | unknown |
| Lmo0379 | 0 | 0 | 0 | 25 | 0 | 85 | 100 | 0 | 0 | 0 | 100 | 151aa/unknwon | unknown |
| Lmo0380 | 0 | 0 | 0 | 25 | 0 | 96 | 92 | 75 | 19 | 0 | 100 | 209aa/unknwon | unknown |
| Lmo0361 | 0 | 0 | 0 | 25 | 0 | 100 | 100 | 75 | 19 | 100 | 0 | 128aa/unknwon | unknown |
| Lmo0409 | 0 | 0 | 0 | 0 | 0 | 78 | 100 | 60 | 78 | 0 | 0 | 821aa/InIF internalin, putative | cell surface proteins |
| | | | | | | | | | | | | peptidoglycan bound protein (LPXTG | |
| | | | | | | | | | | | | motif) | |
| Lmo0444 | 0 | 100 | 0 | 0 | 0 | 37 | 100 | 65 | 0 | 0 | 100 | 926aa/unknown, conserved hypothetical | unknown |
| | | | | | | | | | | | | protein | |
| Lmo0445 | 0 | 100 | 0 | 0 | 0 | 44 | 100 | 65 | 0 | 0 | 100 | 495aa/unknown, similar to transcription | Regulation |
| | | | | | | | | | | | | regulator | |
| Lmo0446 | 0 | 100 | 0 | 0 | 0 | 44 | 92 | 65 | 0 | 0 | 100 | 329aa/unknown, similar to penicillin | Detoxification |
| | | | | | | | | | | | | acylase and to conjugated bile acid | |
| | | | | | | | | | | | | hydrolase | |
| Lmo0447 | 50 | 100 | 50 | 0 | 0 | 44 | 100 | 65 | 0 | 0 | 100 | 462aa/unknown, similar to glutamate | Metabolism of amino acids |
| | | | | | | | | | | | | decarboxylase | and related molecules |
| Lmo0448 | 0 | 100 | 0 | 63 | 0 | 44 | 100 | 95 | 63 | 33 | 100 | 483/unknown, similar to amino acid | Transport/binding proteins |
| | | | | | | | | | | | | antiporter | and lipoproteins |
| Lmo0459 | 0 | 0 | 0 | 0 | 0 | 48 | 100 | 100 | 100 | 100 | 50 | 512aa/unknown, similar to transcription | Regulation |
| | | | | | | | | | | | | regulator (VirR from Streptococcus | |
| | | | | | | | | | | | | pyogenes) | |
| Lmo0460 | 0 | 0 | 0 | 0 | 0 | 48 | 100 | 100 | 100 | 100 | 50 | 689aa/unknown, putative membrane | cell surface proteins |
| | | | | | | | | | | | | associated lipoprotein | |
| Lmo0462 | 0 | 0 | 0 | 0 | 0 | 48 | 92 | 100 | 100 | 100 | 50 | 161aa/unknwon | unknown |
| Lmo0472 | 0 | 50 | 0 | 88 | 0 | 81 | 100 | 25 | 4 | 0 | 100 | 295aa/unknwon | unknown |
| Lmo0473 | 67 | 50 | 0 | 38 | 0 | 93 | 100 | 20 | 67 | 100 | 100 | 361aa/unknwon | unknown |
| Lmo0474 | 0 | 0 | 0 | 25 | 0 | 93 | 100 | 10 | 100 | 0 | 0 | 115aa/unknwon | unknown |
| Lmo0477 | 17 | 50 | 0 | 63 | 0 | 89 | 100 | 20 | 63 | 0 | 50 | 125aa/unknown, putative secreted protein | unknown |
| Lmo0478 | 33 | 50 | 0 | 75 | 0 | 93 | 100 | 25 | 63 | 0 | 50 | 125aa/unknown, putative secreted protein | unknown |
| Lmo0492 | 0 | 0 | 0 | 0 | 0 | 85 | 100 | 95 | 100 | 0 | 50 | 302aa/unknown, similar to transcriptional | Regulation |
| | | | | | | | | | | | | regulator (LysR family) | |
| Lmo0493 | 67 | 0 | 100 | 0 | 0 | 85 | 100 | 100 | 100 | 0 | 50 | 586aa/unknown, similar to acylase | Detoxification |
| Lmo0630 | 50 | 100 | 0 | 0 | 0 | 100 | 100 | 95 | 81 | 0 | 50 | 676aa/unknown, similar to transcription | Regulation |
| | | | | | | | | | | | | antiterminator BglG family | |
| Lmo0631 | 0 | 100 | 0 | 0 | 0 | 100 | 100 | 100 | 81 | 0 | 50 | 150aa/unknown, similar to PTS system, | Transport/binding proteins |
| | | | | | | | | | | | | fructose-specific IIA component | and lipoproteins |
| Lmo0632 | 100 | 100 | 0 | 0 | 0 | 100 | 100 | 100 | 81 | 0 | 50 | 353aa/unknown, similar to PTS system, | Transport/binding proteins |
| | | | | | | | | | | | | fructose-specific IIC component | and lipoproteins |
| Lmo0633 | 100 | 100 | 0 | 0 | 0 | 100 | 100 | 95 | 81 | 0 | 50 | 102aa/unknown, similar to PTS system, | Transport/binding proteins |
| | | | | | | | | | | | | fructose-specific IIB component | and lipoproteins |
| Lmo0634 | 83 | 100 | 0 | 0 | 0 | 100 | 100 | 100 | 81 | 0 | 50 | 422aa/unknown, similar to an E. coli | Specific pathways |
| | | | | | | | | | | | | putative tagatose 6-phosphate kinase | |
| Lmo0745 | 0 | 0 | 0 | 0 | 0 | 96 | 100 | 0 | 0 | 100 | 0 | 167aa/unknown | unknown |
| Lmo0750 | 0 | 0 | 0 | 0 | 0 | 100 | 100 | 0 | 4 | 100 | 0 | 171aa/unknwon | unknown |
| Lmo0751 | 17 | 0 | 0 | 0 | 0 | 96 | 100 | 60 | 81 | 100 | 0 | 98aa/unknwon | unknown |
| Lmo0780 | 33 | 50 | 0 | 0 | 0 | 100 | 100 | 15 | 7 | 100 | 100 | 115aa/unknown | unknown |
| Lmo0801 | 0 | 50 | 50 | 13 | 0 | 70 | 100 | 0 | 30 | 33 | 0 | 646aa/unknown, similar to internalin, | cell surface proteins |
| | | | | | | | | | | | | putative peptidoglycan bound protein | |
| | | | | | | | | | | | | (LPXTG motif) | |
| Lmo0804 | 0 | 0 | 0 | 0 | 0 | 44 | 100 | 45 | 33 | 100 | 100 | 156aa/Unknwon | unknown |
| Lmo0842 | 17 | 100 | 0 | 75 | 0 | 100 | 100 | 95 | 48 | 33 | 0 | 2044aa/unknown, surface protein (LPXTG | cell surface proteins |
| | | | | | | | | | | | | motif) | |
| Lmo1032 | 100 | 0 | 0 | 0 | 0 | 100 | 92 | 100 | 100 | 0 | 100 | 274aa/unknown, similar to transketoiase | Specific pathways |
| Lmo1099 | 17 | 0 | 0 | 0 | 0 | 11 | 50 | 5 | 19 | 0 | 50 | 139aa/unknown, similar to a protein | Transposon and IS |
| | | | | | | | | | | | | encoded by Tn916 | |
| Lmo1106 | 33 | 0 | 0 | 50 | 0 | 63 | 67 | 35 | 30 | 33 | 100 | 816aa/unknown, highly similar to TN916 | Transposon and IS |
| | | | | | | | | | | | | ORF16 | |
| Lmo1114 | 100 | 100 | 50 | 13 | 0 | 81 | 92 | 60 | 74 | 33 | 100 | 104aa/unknown, highly similar to TN916 | Transposon and IS |
| | | | | | | | | | | | | ORF23 | |
| Lmo1115 | 33 | 0 | 50 | 13 | 0 | 78 | 75 | 10 | 4 | 33 | 100 | 862aa/unknown, similar to fibrinogen- | cell surface proteins |
| | | | | | | | | | | | | binding protein (LPXTG motif) | |
| Lmo1116 | 67 | 0 | 0 | 63 | 0 | 48 | 100 | 60 | 81 | 0 | 50 | 257aa/unknown, similar to regulatory | Regulation |
| | | | | | | | | | | | | proteins | |
| Lmo1117 | 67 | 0 | 0 | 50 | 0 | 52 | 100 | 60 | 81 | 0 | 50 | 118aa/unknown | unknown |
| Lmo1120 | 67 | 50 | 50 | 25 | 0 | 85 | 100 | 95 | 85 | 0 | 0 | 138aa/unknwon | unknown |
| Lmo1121 | 17 | 100 | 0 | 50 | 0 | 96 | 100 | 95 | 85 | 33 | 50 | 122aa/unknwon | unknown |
| Lmo1125 | 67 | 50 | 0 | 25 | 0 | 100 | 100 | 0 | 0 | 100 | 100 | 177aa/unknown | unknown |
| Lmo1307 | 33 | 50 | 0 | 0 | 0 | 100 | 100 | 0 | 0 | 100 | 100 | 280aa/unknwon | unknown |
| Lmo1597 | 67 | 100 | 0 | 0 | 0 | 63 | 100 | 85 | 19 | 0 | 0 | 259aa/unknwon | unknown |
| Lmo1604 | 33 | 0 | 0 | 75 | 0 | 100 | 100 | 75 | 67 | 33 | 100 | 181aa/unknown | unknown |
| Lmo1648 | 17 | 50 | 0 | 25 | 0 | 78 | 100 | 20 | 7 | 0 | 50 | 120aa/unknwon | unknown |
| Lmo1714 | 0 | 0 | 0 | 25 | 0 | 41 | 100 | 100 | 100 | 100 | 0 | 190aa/unknown | unknown |
| Lmo2026 | 0 | 0 | 0 | 0 | 0 | 41 | 100 | 0 | 0 | 0 | 0 | 626aa/unknown, peptidoglycan linked | cell surface proteins |
| | | | | | | | | | | | | proteins (LPXTG) | |
| Lmo2027 | 0 | 0 | 0 | 0 | 0 | 93 | 100 | 75 | 70 | 0 | 100 | 367aa/unknown, putative cell surface | cell surface proteins |
| | | | | | | | | | | | | protein, similar to internalin proteins | |
| Lmo2276 | 0 | 0 | 0 | 0 | 0 | 78 | 75 | 70 | 67 | 0 | 0 | 165aa/unknown, similar to an unknown | Phage-related functions |
| | | | | | | | | | | | | bacteriophage protein | |
| Lmo2277 | 17 | 0 | 0 | 25 | 0 | 56 | 42 | 10 | 11 | 33 | 50 | 183aa/unknwon | unknown |
| Lmo2278 | 0 | 0 | 50 | 0 | 0 | 89 | 100 | 90 | 0 | 0 | 0 | 281aa/LysA: L-alanoyl-D-glutamate | Phage-related functions |
| | | | | | | | | | | | | peptidase | |
| Lmo2282 | 0 | 0 | 0 | 25 | 0 | 33 | 25 | 55 | 4 | 33 | 0 | 105aa/protein gp21 [Bacteriophage A118] | Phage-related functions |
| Lmo2283 | 0 | 0 | 0 | 0 | 0 | 37 | 25 | 55 | 0 | 0 | 0 | 357aa/protein gp20 [Bacteriophage A118] | Phage-related functions |
| Lmo2296 | 0 | 0 | 0 | 25 | 0 | 15 | 33 | 15 | 26 | 100 | 0 | 333aa/unknown, similar to coat protein | Phage-related functions |
| | | | | | | | | | | | | [Bacteriophage SPP1] | |
| Lmo2311 | 0 | 0 | 0 | 0 | 0 | 41 | 75 | 5 | 0 | 100 | 50 | 153aa/unknwon | unknown |
| Lmo2312 | 0 | 50 | 0 | 25 | 0 | 56 | 75 | 80 | 33 | 100 | 0 | 156aa/unknwon | unknown |
| Lmo2313 | 0 | 50 | 0 | 13 | 0 | 59 | 50 | 45 | 33 | 67 | 0 | 147aa/unknown, similar to a bacteriophage | Phage-related functions |
| | | | | | | | | | | | | protein | |
| Lmo2319 | 0 | 50 | 50 | 13 | 0 | 33 | 92 | 15 | 19 | 0 | 0 | 158aa/unknown, similar to a bacteriophage | Phage-related functions |
| | | | | | | | | | | | | proteins | |
| Lmo2322 | 0 | 0 | 0 | 63 | 0 | 48 | 50 | 25 | 0 | 0 | 0 | 71aa/gp44 [Bacteriophage A118] | Phage-related functions |
| Lmo2324 | 0 | 50 | 0 | 38 | 0 | 63 | 83 | 35 | 41 | 67 | 0 | 258aa/unknown, similar to anti-repressor | Regulation |
| | | | | | | | | | | | | [Bacteriophage A118] | |
| Lmo2408 | 0 | 100 | 0 | 0 | 0 | 37 | 100 | 0 | 0 | 0 | 0 | 67aa/unknown, similar to repressor | Regulation |
| | | | | | | | | | | | | protein | |
| Lmo2409 | 0 | 50 | 0 | 0 | 0 | 33 | 100 | 0 | 0 | 0 | 0 | 137aa/unknwon | unknown |
| Lmo2410 | 0 | 0 | 0 | 0 | 0 | 33 | 100 | 0 | 0 | 0 | 0 | 183aa/unknown | unknown |
| Lmo2443 | 0 | 0 | 0 | 0 | 0 | 89 | 100 | 95 | 100 | 100 | 0 | 139aa/unknwon | unknown |
| Lmo2576 | 0 | 0 | 0 | 0 | 0 | 100 | 100 | 0 | 0 | 0 | 100 | 1530aa/unknwon, peptidoglycan anchored | cell surface proteins |
| | | | | | | | | | | | | protein (LPXTG motif) | |
| Lmo2594 | 0 | 0 | 0 | 50 | 0 | 74 | 92 | 80 | 100 | 33 | 100 | 119aa/unknwon | unknown |
| Lmo2595 | 0 | 0 | 0 | 0 | 0 | 52 | 100 | 40 | 100 | 100 | 50 | 289aa/unknown, similar to unknown | unknown |
| | | | | | | | | | | | | proteins | |
| Lmo2671 | 17 | 100 | 50 | 0 | 0 | 100 | 100 | 100 | 100 | 0 | 0 | 122aa/unknwon | unknown |
| Lmo2686 | 17 | 0 | 0 | 0 | 0 | 93 | 100 | 5 | 0 | 0 | 50 | 177aa/unknwon | unknown |
| Lmo2786 | 0 | 0 | 0 | 0 | 0 | 100 | 100 | 0 | 0 | 0 | 100 | 327aa/bvrC: unknown | unknown |
| Lmo2787 | 0 | 0 | 0 | 0 | 0 | 100 | 100 | 0 | 0 | 0 | 100 | 640aa/bvrB-beta-glucoside-specific | Transport/binding proteins |
| | | | | | | | | | | | | phosphotransferase enzyme II | and lipoproteins |
| Lmo2807 | 17 | 0 | 50 | 13 | 0 | 63 | 100 | 15 | 48 | 67 | 0 | 127aa/unknown, hypothetical secreted | unknown |
| | | | | | | | | | | | | protein | |
| Lmo2809 | 0 | 0 | 50 | 25 | 0 | 70 | 92 | 100 | 44 | 0 | 0 | 126aa/unknown, hypothetical secreted | unknown |
| | | | | | | | | | | | | protein | |
| Lmo0036 | 100 | 0 | 50 | 0 | 100 | 100 | 100 | 100 | 100 | 0 | 0 | 341aa/unknown, similar to ornithine | Metabolism of amino acids |
| | | | | | | | | | | | | carbamoyltransferase | and related molecules |
| Lmo0037 | 100 | 0 | 50 | 0 | 0 | 100 | 100 | 100 | 100 | 0 | 0 | 461aa/unknown, similar to amino acid | Transport/binding proteins |
| | | | | | | | | | | | | transporter | and lipoproteins |
| Lmo0038 | 100 | 0 | 50 | 0 | 0 | 100 | 100 | 100 | 100 | 0 | 0 | 363aa/unknown, conserved hypothetical | unknown |
| | | | | | | | | | | | | protein | |
| Lmo0039 | 100 | 0 | 50 | 0 | 0 | 100 | 100 | 100 | 100 | 0 | 0 | 313aa/carbamate kinase | Metabolism of amino acids |
| | | | | | | | | | | | | | and related molecules |
| Lmo0040 | 33 | 0 | 50 | 0 | 0 | 100 | 100 | 100 | 100 | 0 | 0 | 369aa/unknown, conserved hypothetical | Phage-related functions |
| | | | | | | | | | | | | protein | |
| Lmo0041 | 50 | 0 | 0 | 0 | 0 | 100 | 100 | 100 | 100 | 0 | 0 | 259aa/unknown, conserved hypothetical | Regulation |
| | | | | | | | | | | | | protein, hypothetical regulator | |
| Lmo0084 | 67 | 50 | 0 | 25 | 0 | 100 | 100 | 100 | 100 | 100 | 100 | 327aa/unknwon, similar to | Specific pathways |
| | | | | | | | | | | | | oxidoreductases | |
| Lmo0106 | 33 | 50 | 100 | 0 | 0 | 96 | 100 | 35 | 96 | 100 | 100 | 300aa/unknown, similar to transcription | Regulation |
| | | | | | | | | | | | | regulator | |
| Lmo0110 | 33 | 100 | 50 | 0 | 0 | 100 | 100 | 100 | 100 | 100 | 100 | 371aa/unknown, similar to lipase | Metabolism of lipids |
| Lmo0147 | 83 | 50 | 50 | 63 | 0 | 100 | 100 | 65 | 85 | 67 | 100 | 145aa/unknwon | unknown |
| Lmo0175 | 0 | 0 | 0 | 100 | 0 | 100 | 100 | 100 | 100 | 100 | 100 | 415aa/unknown, putative peptidoglycan | cell surface proteins |
| | | | | | | | | | | | | bound protein (LPXTG motif) | |
| Lmo0461 | 83 | 0 | 100 | 0 | 0 | 81 | 92 | 100 | 100 | 100 | 100 | 157aa/unknwon | unknown |
| Lmo0463 | 0 | 0 | 0 | 0 | 0 | 56 | 100 | 100 | 100 | 100 | 50 | 101aa/putative peptidoglycan linked | cell surface proteins |
| | | | | | | | | | | | | protein (LPXAG motif) | |
| Lmo0476 | 67 | 100 | 50 | 75 | 0 | 93 | 100 | 100 | 100 | 100 | 50 | 325aa/unknown, similar to oxetanocin A | Detoxification |
| | | | | | | | | | | | | resistance protein oxrB | |
| Lmo0610 | 17 | 0 | 0 | 50 | 0 | 100 | 100 | 95 | 100 | 67 | 100 | 589aa/unknown, similar to internalin | cell surface proteins |
| | | | | | | | | | | | | proteins, putative peptidoglycan bound | |
| | | | | | | | | | | | | protein (LPXTG motif) | |
| Lmo0638 | 0 | 0 | 0 | 0 | 0 | 100 | 100 | 100 | 100 | 33 | 100 | 209aa/unknown | unknown |
| Lmo0673 | 83 | 0 | 50 | 75 | 0 | 100 | 100 | 100 | 100 | 100 | 100 | 69aa/unknwon | unknown |
| Lmo0732 | 50 | 50 | 0 | 75 | 0 | 100 | 100 | 80 | 70 | 100 | 100 | 638aa/unknown, putative peptidoglycan | cell surface proteins |
| | | | | | | | | | | | | bound protein (LPXTG motif) | |
| Lmo0838 | 100 | 0 | 100 | 0 | 0 | 100 | 100 | 100 | 100 | 100 | 100 | 461aa/unknown, highly similar to hexose | Transport/binding proteins |
| | | | | | | | | | | | | phosphate transport protein | and lipoproteins |
| Lmo0849 | 33 | 0 | 0 | 13 | 0 | 100 | 100 | 100 | 100 | 33 | 100 | 616aa/unknown, similar to amidases | Cell wall |
| Lmo1102 | 100 | 0 | 50 | 13 | 0 | 78 | 100 | 65 | 76 | 100 | 100 | 111aa/unknown, similar to cadmium efflux | Detoxification |
| | | | | | | | | | | | | system accessory proteins | |
| Lmo1134 | 100 | 0 | 100 | 0 | 0 | 100 | 100 | 100 | 100 | 100 | 100 | 115aa/unknown, similar to regulatory | Regulation |
| | | | | | | | | | | | | proteins | |
| Lmo1247 | 0 | 0 | 0 | 100 | 0 | 100 | 100 | 100 | 100 | 100 | 100 | 87aa/unknwon | unknown |
| Lmo1263 | 50 | 0 | 0 | 13 | 0 | 100 | 100 | 100 | 100 | 100 | 100 | 71aa/unknown, similar to transcriptional | Regulation |
| | | | | | | | | | | | | regulator | |
| Lmo1413 | 0 | 0 | 50 | 0 | 0 | 100 | 100 | 100 | 100 | 100 | 100 | 439aa/unknown, putative peptidoglycan | cell surface proteins |
| | | | | | | | | | | | | bound protein (LPXTG motif) | |
| Lmo1478 | 33 | 0 | 0 | 0 | 0 | 100 | 100 | 100 | 100 | 100 | 100 | 145aa/unknown, similar to transcriptional | Regulation |
| | | | | | | | | | | | | regulator (MerR family) | |
| Lmo1656 | 100 | 100 | 50 | 50 | 0 | 100 | 100 | 100 | 100 | 100 | 100 | 143aa/unknwon | unknown |
| Lmo2045 | 0 | 0 | 50 | 50 | 0 | 100 | 100 | 100 | 100 | 100 | 100 | 128aa/unknwon | unknown |
| Lmo2067 | 50 | 0 | 100 | 0 | 0 | 100 | 100 | 100 | 100 | 100 | 100 | 325aa/unknown, similar to conjugated bile | Detoxification |
| | | | | | | | | | | | | acid hydrolase | |
| Lmo2085 | 0 | 50 | 50 | 0 | 0 | 100 | 100 | 100 | 100 | 100 | 100 | 562aa/unknown, putative peptidoglycan | cell surface proteins |
| | | | | | | | | | | | | bound protein (LPXTG motif) | |
| Lmo2143 | 17 | 0 | 0 | 0 | 0 | 100 | 100 | 100 | 100 | 100 | 100 | 580aa/unknown, weakly similar to | Specific pathways |
| | | | | | | | | | | | | mannose-6-phosphate isomerase | |
| Lmo2144 | 17 | 0 | 0 | 0 | 0 | 100 | 100 | 100 | 100 | 100 | 100 | 244aa/unknown, similar to transcription | Regulation |
| | | | | | | | | | | | | regulator GntR family | |
| Lmo2178 | 17 | 100 | 0 | 100 | 0 | 100 | 100 | 100 | 100 | 100 | 100 | 1612aa/unknown, putative peptidoglycan | cell surface proteins |
| | | | | | | | | | | | | bound protein (LPXTG motif) | |
| Lmo2197 | 17 | 0 | 100 | 25 | 0 | 100 | 100 | 100 | 100 | 100 | 100 | 114aa/unknwon | unknown |
| Lmo2387 | 33 | 0 | 0 | 0 | 0 | 100 | 100 | 100 | 100 | 100 | 100 | 408aa/unknown, conserved hypothetical | unknown |
| | | | | | | | | | | | | protein | |
| Lmo2567 | 0 | 100 | 0 | 100 | 0 | 100 | 100 | 100 | 100 | 100 | 100 | 66aa/unknwon | unknown |
| Lmo2731 | 100 | 100 | 50 | 100 | 0 | 100 | 100 | 100 | 100 | 100 | 100 | 122aa/unknown, similar to transcription | Regulation |
| | | | | | | | | | | | | regulator (RpiR family) | |
| Lmo2735 | 33 | 0 | 0 | 25 | 0 | 100 | 100 | 100 | 100 | 100 | 100 | 560aa/unknown, similar to Sucrose | Specific pathways |
| | | | | | | | | | | | | phosphorylase | |
| Lmo2771 | 17 | 100 | 50 | 0 | 0 | 100 | 100 | 100 | 100 | 0 | 100 | 482aa/unknown, similar to beta- | Specific pathways |
| | | | | | | | | | | | | glucosidase | |
| Lmo2773 | 0 | 100 | 0 | 0 | 0 | 100 | 100 | 100 | 100 | 0 | 100 | 279aa/unknwon, similar to transcription | Regulation |
| | | | | | | | | | | | | antiterminator | |
| Lmo2776 | 0 | 0 | 0 | 63 | 0 | 74 | 100 | 100 | 100 | 33 | 100 | 131aa/unknwon | unknown |
| Lmo2781 | 50 | 100 | 50 | 0 | 0 | 100 | 100 | 100 | 100 | 100 | 100 | 756aa/unknown, similar to beta- | Specific pathways |
| | | | | | | | | | | | | glucosidase | |
| Lmo2782 | 67 | 100 | 0 | 0 | 0 | 100 | 100 | 100 | 100 | 100 | 100 | 99aa/unknown, similar to PTS, cellobiose- | Transport/binding proteins |
| | | | | | | | | | | | | specific IIB component | and lipoproteins |
| Lmo0257 | 100 | 100 | 50 | 50 | 0 | 100 | 100 | 100 | 100 | 100 | 100 | 392aa/unknown, similar to unknown | unknown |
| | | | | | | | | | | | | protein | |
| Lmo0514 | 67 | 100 | 100 | 68 | 0 | 100 | 100 | 100 | 100 | 100 | 100 | 605aa/unknown, similar to internalin | cell surface proteins |
| | | | | | | | | | | | | proteins, putative peptidoglycan bound | |
| | | | | | | | | | | | | protein (LPXTG motif) | |
| Lmo0843 | 100 | 100 | 100 | 100 | 0 | 100 | 100 | 100 | 100 | 100 | 100 | 85aa/unknown, similar to B. subtilis | unknown |
| | | | | | | | | | | | | protein YsdA | |
| Lmo0880 | 83 | 100 | 100 | 88 | 0 | 100 | 100 | 100 | 100 | 100 | 100 | 462aa/unknown, similar to wall associated | cell surface proteins |
| | | | | | | | | | | | | protein precursor (LPXTG motif) | |
| Lmo0932 | 100 | 100 | 100 | 100 | 0 | 100 | 100 | 100 | 100 | 100 | 100 | 201aa/unknown, conserved hypothetical | unknown |
| | | | | | | | | | | | | protein | |
| Lmo1136 | 100 | 100 | 100 | 100 | 100 | 100 | 100 | 100 | 100 | 100 | 100 | 539aa/unknown, similar to Internalin, | cell surface proteins |
| | | | | | | | | | | | | putative peptidoglycan bound protein | |
| | | | | | | | | | | | | (LPXTG motif) | |
| Lmo1289 | 83 | 50 | 100 | 63 | 50 | 100 | 100 | 100 | 100 | 100 | 100 | 593aa/unknown, similar to Internalin | cell surface proteins |
| | | | | | | | | | | | | proteins, putative peptidoglycan bound | |
| | | | | | | | | | | | | protein (LPXTG motif) | |
| Lmo1302 | 100 | 100 | 100 | 100 | 50 | 100 | 100 | 100 | 100 | 100 | 100 | 204aa/unknown, highly similar to SOS | Regulation |
| | | | | | | | | | | | | response regulator lexA, transcription | |
| | | | | | | | | | | | | repressor protein | |
| Lmo1303 | 100 | 100 | 100 | 88 | 50 | 100 | 100 | 100 | 100 | 100 | 100 | 109aa/unknown, similar to B. subtilis YneA | unknown |
| | | | | | | | | | | | | protein | |
| Lmo1700 | 83 | 100 | 100 | 100 | 0 | 100 | 100 | 100 | 100 | 100 | 100 | 68aa/unknwon | unknown |
| Lmo1707 | 100 | 100 | 100 | 100 | 0 | 100 | 100 | 100 | 100 | 100 | 100 | 73aa/unknwon | unknown |
| Lmo1799 | 100 | 100 | 50 | 88 | 0 | 100 | 100 | 100 | 100 | 100 | 100 | 903aa/unknown, putative peptidoglycan | cell surface proteins |
| | | | | | | | | | | | | bound protein (LPXTG motif) | |
| Lmo2451 | 100 | 100 | 100 | 100 | 0 | 100 | 100 | 100 | 100 | 100 | 100 | 77aa/unknown, similar to preprotein | Protein secretion |
| | | | | | | | | | | | | translocase subunit SecG | |
| Lmo2548 | 100 | 100 | 100 | 100 | 0 | 100 | 100 | 100 | 100 | 100 | 100 | 81aa/rpmE: ribosomal protein L31 | Ribosomal proteins |
| Lmo2772 | 33 | 100 | 100 | 0 | 0 | 100 | 100 | 100 | 100 | 0 | 100 | 617aa/unknown, similar to beta-glucoside- | Transport/binding proteins |
| | | | | | | | | | | | | specific enzyme IIABC | and lipoproteins |
| Lmo1097 | 0 | 0 | 0 | 13 | 0 | 11 | 0 | 5 | 0 | 0 | 0 | 400aa/unknown, similar to integrases | Transposon and IS |
| Lmo1101 | 0 | 0 | 0 | 13 | 0 | 19 | 8 | 5 | 7 | 0 | 0 | 166aa/unknown, similar to lipoprotein | Protein secretion |
| | | | | | | | | | | | | signal peptidase | |
| Lmo1103 | 0 | 0 | 0 | 0 | 50 | 11 | 8 | 0 | 0 | 0 | 0 | 305aa/unknown, highly similar to TN916 | Transposon and IS |
| | | | | | | | | | | | | ORF13 | |
| Lmo1104 | 0 | 0 | 0 | 25 | 0 | 7 | 0 | 0 | 0 | 0 | 0 | 336aa/unknown, highly similar to TN916 | Cell wall |
| | | | | | | | | | | | | ORF14 and to L. monocytogenes P60 | |
| | | | | | | | | | | | | protein | |
| Lmo1105 | 0 | 0 | 0 | 0 | 0 | 7 | 0 | 0 | 0 | 0 | 0 | 719aa/unknown, highly similar to TN916 | Transposon and IS |
| | | | | | | | | | | | | ORF15 | |
| Lmo1107 | 0 | 0 | 0 | 38 | 0 | 7 | 8 | 0 | 0 | 0 | 0 | 131aa/unknown, highly similar to TN916 | Transposon and IS |
| | | | | | | | | | | | | ORF17 | |
| Lmo1108 | 0 | 0 | 0 | 0 | 0 | 11 | 8 | 0 | 0 | 0 | 0 | 166aa/unknown, highly similar to TN916 | Transposon and IS |
| | | | | | | | | | | | | ORF18 | |
| Lmo1110 | 0 | 0 | 0 | 0 | 0 | 7 | 8 | 0 | 0 | 0 | 0 | 96aa/unknown, similar to unknown | unknown |
| | | | | | | | | | | | | proteins | |
| Lmo1111 | 0 | 0 | 0 | 0 | 0 | 7 | 8 | 0 | 0 | 0 | 0 | 394aa/unknown, highly similar to TN916 | Transposon and IS |
| | | | | | | | | | | | | ORF20 | |
| Lmo1112 | 17 | 50 | 0 | 13 | 0 | 15 | 17 | 5 | 7 | 0 | 0 | 466aa/unknown, highly similar to TN916 | Transposon and IS |
| | | | | | | | | | | | | ORF21 | |
| Lmo1113 | 0 | 0 | 0 | 13 | 0 | 11 | 25 | 5 | 0 | 0 | 0 | 124aa/unknown, highly similar to TN916 | Transposon and IS |
| | | | | | | | | | | | | ORF22 | |
| Lmo2301 | 0 | 0 | 0 | 0 | 0 | 22 | 17 | 0 | 0 | 0 | 0 | 264aa/unknown, similar to putative | Phage-related functions |
| | | | | | | | | | | | | terminase small subunit from | |
| | | | | | | | | | | | | Bacteriophage A118 | |
| Lmo2302 | 0 | 0 | 0 | 0 | 0 | 7 | 0 | 30 | 15 | 0 | 0 | 179aa/unknown | unknown |
| Lmo2318 | 0 | 0 | 0 | 0 | 0 | 4 | 0 | 5 | 7 | 0 | 0 | 232aa/unknwon | unknown |
| Lmo2331 | 0 | 0 | 0 | 0 | 0 | 22 | 25 | 15 | 0 | 0 | 0 | 226aa/unknown, weakly similar to | Phage-related functions |
| | | | | | | | | | | | | gp32_Bacteriophage A118 protein | |

b: Percentage of presence of a specific gene in the different Listeria species and L. monocytogenes sub-groups
L. monocytogenes (CLIP 80459)

| | | | | | | | | | | | | | |
|---|---|---|---|---|---|---|---|---|---|---|---|---|---|
| ORF 2372 | 33 | 100 | 0 | 0 | 0 | 0 | 0 | 0 | 100 | 0 | 0 | 596pb/unknown, similar titelchoic | cell wall |
| | | | | | | | | | | | | acid biosynthesis | |
| | | | | | | | | | | | | protein C | |
| ORF 2110 | 67 | 50 | 0 | 0 | 0 | 0 | 0 | 0 | 100 | 0 | 0 | 597pb/unknown, similar to | unknown |
| | | | | | | | | | | | | hypothetical protein | |
| ORF 799 | 0 | 50 | 0 | 63 | 0 | 7 | 0 | 0 | 100 | 0 | 0 | 453pb/unknown | unknown |
| ORF 3840 | 17 | 0 | 0 | 0 | 0 | 0 | 0 | 100 | 100 | 0 | 0 | 593pb/unknown, similar to | Regulation |
| | | | | | | | | | | | | transcription regulator | |
| ORF 2568-2569 | 50 | 0 | 0 | 0 | 0 | 0 | 0 | 100 | 100 | 0 | 0 | 597pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif)-unknown | |
| ORF 2017a | 0 | 0 | 0 | 0 | 0 | 0 | 0 | 100 | 100 | 0 | 0 | 599pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 29 | 0 | 0 | 0 | 0 | 0 | 0 | 0 | 95 | 100 | 0 | 0 | 609pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 2819 | 0 | 0 | 0 | 0 | 0 | 0 | 0 | 100 | 100 | 0 | 0 | 562pb/unknown, similar to | Regulation |
| | | | | | | | | | | | | transcriptional regulator | |
| ORF 2541 | 0 | 0 | 0 | 0 | 0 | 30 | 0 | 100 | 100 | 0 | 0 | 590pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 2017b | 50 | 0 | 0 | 25 | 0 | 26 | 0 | 100 | 100 | 33 | 100 | 285pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 3401 | 33 | 0 | 0 | 25 | 0 | 11 | 0 | 50 | 100 | 33 | 0 | 575pb/unknown | unknown |
| ORF 2074 | 100 | 100 | 50 | 50 | 0 | 48 | 0 | 100 | 100 | 100 | 0 | 599pb/unknown similar to | Specific pathways |
| | | | | | | | | | | | | oligo-1,6-glucosidase | |
| ORF528-529 | 0 | 0 | 0 | 13 | 0 | 0 | 0 | 10 | 100 | 0 | 0 | 591pb/unknown-unknown | unknown |
| ORF 150 | 100 | 100 | 100 | 63 | 100 | 89 | 92 | 95 | 100 | 100 | 100 | 393pb/unknown, similar to | cell wall |
| | | | | | | | | | | | | amidase | |
| ORF 2777 | 50 | 50 | 100 | 0 | 0 | 33 | 0 | 100 | 100 | 0 | 0 | 600pb/unknown | unknown |
| ORF 2759 | 100 | 100 | 50 | 38 | 0 | 52 | 0 | 95 | 100 | 100 | 0 | 601pb/unknown, sucrose | Specific pathways |
| | | | | | | | | | | | | phosphorylase | |
| ORF 75 | 100 | 100 | 100 | 100 | 100 | 100 | 100 | 100 | 100 | 100 | 100 | 504pb/unknown | unknown |
| ORF 660 | 0 | 0 | 0 | 0 | 0 | 33 | 0 | 10 | 100 | 0 | 0 | 603pb/unknown | unknown |
| ORF 2885 | 0 | 100 | 0 | 63 | 0 | 0 | 0 | 0 | 100 | 100 | 50 | 592pb/unknown | unknown |
| ORF 1445 | 50 | 0 | 0 | 0 | 0 | 0 | 0 | 0 | 100 | 100 | 100 | 607pb/unknown, conserved | unknown |
| | | | | | | | | | | | | hypothetical protein ykcB - | |
| | | | | | | | | | | | | Bacillus subtilis | |
| ORF 1590 | 0 | 0 | 0 | 0 | 0 | 100 | 0 | 95 | 100 | 0 | 100 | 588pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 1491 | 33 | 100 | 100 | 0 | 0 | 0 | 0 | 100 | 100 | 0 | 100 | 598pb/unknown, similar to | unknown |
| | | | | | | | | | | | | hypothetical protein | |
| ORF 1490 | 33 | 100 | 100 | 0 | 0 | 0 | 0 | 100 | 100 | 0 | 100 | 600pb/unknown, similar to | Transport/binding |
| | | | | | | | | | | | | sucrose-specific PTS system | proteins and |
| | | | | | | | | | | | | IIBC component | lipoproteins |
| ORF 1391 | 67 | 100 | 100 | 88 | 0 | 100 | 100 | 100 | 100 | 100 | 100 | 600pb/unknown | unknown |
| ORF 3792 | 17 | 0 | 0 | 0 | 0 | 67 | 17 | 100 | 100 | 100 | 100 | 594pb/unknown | unknown |
| ORF 4101 | 67 | 100 | 0 | 75 | 0 | 59 | 42 | 95 | 100 | 33 | 100 | 613pb/unknown, similar to | Transport/binding |
| | | | | | | | | | | | | similar to ABC transporter | proteins and |
| | | | | | | | | | | | | | lipoproteins |
| ORF 1490 | 33 | 100 | 100 | 0 | 100 | 4 | 0 | 100 | 100 | 0 | 100 | 474pb/unknown, similar to | Transport/binding |
| | | | | | | | | | | | | sucrose phosphotransferase | proteins and |
| | | | | | | | | | | | | enzyme IIBC component | lipoproteins |
| ORF2969-2968 | 83 | 100 | 100 | 88 | 0 | 93 | 100 | 90 | 100 | 100 | 100 | 583pb/unknown-unknown | unknown |
| ORF 148-149 | 0 | 0 | 0 | 0 | 0 | 0 | 0 | 10 | 100 | 0 | 0 | 620pb/unknown-unknown, | cell wall |
| | | | | | | | | | | | | similar to amidase | |
| ORF 3175 | 100 | 100 | 100 | 100 | 0 | 100 | 100 | 100 | 100 | 100 | 100 | 589pb/unknown, similar to | Phage-related |
| | | | | | | | | | | | | phage protein | functions |
| ORF 2858 | 50 | 50 | 50 | 50 | 100 | 74 | 75 | 90 | 100 | 33 | 100 | 249pb/unknown, similar to | unknown |
| | | | | | | | | | | | | hypothetical protein | |
| ORF 2541 | 17 | 0 | 0 | 0 | 0 | 41 | 0 | 95 | 100 | 0 | 100 | 351pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 757 | 0 | 0 | 0 | 38 | 0 | 4 | 0 | 35 | 100 | 0 | 0 | 522pb/unknown | unknown |
| ORF 2076 | 100 | 100 | 50 | 50 | 0 | 48 | 0 | 100 | 100 | 100 | 0 | 597pb/unknown, similar to | Transport/binding |
| | | | | | | | | | | | | maltodextrin ABC | proteins and |
| | | | | | | | | | | | | transporter | lipoproteins |
| ORF 2013 | 0 | 0 | 0 | 0 | 0 | 37 | 0 | 95 | 100 | 100 | 0 | 597pb/unknown, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF 1228 | 17 | 0 | 0 | 0 | 0 | 7 | 0 | 100 | 96 | 0 | 0 | 599pb/unknown, similar to | Transport/binding |
| | | | | | | | | | | | | similar to ABC transporter | proteins and |
| | | | | | | | | | | | | | lipoproteins |
| ORF 3184 | 33 | 50 | 0 | 25 | 0 | 26 | 33 | 70 | 96 | 33 | 50 | 597pb/unknown | unknown |
| ORF 2013 | 0 | 0 | 0 | 0 | 0 | 26 | 0 | 85 | 96 | 100 | 0 | 595pb/unknwon, similar to | cell surface proteins |
| | | | | | | | | | | | | internalin proteins, putative | |
| | | | | | | | | | | | | peptidoglycan bound protein | |
| | | | | | | | | | | | | (LPXTG motif) | |
| ORF1912-1913 | 33 | 50 | 50 | 0 | 0 | 26 | 8 | 15 | 93 | 100 | 100 | 602pb/unknown-unknown | unknown |
| ORF 2262 | 0 | 0 | 0 | 0 | 0 | 30 | 0 | 15 | 93 | 0 | 0 | 601pb/unknown | unknown |
| ORF 72-73-74 | 0 | 0 | 0 | 0 | 0 | 26 | 0 | 35 | 78 | 100 | 50 | 598pb/unknown-unknown | unknown |
| | | | | | | | | | | | | unknown | |
| ORF 2815 | 0 | 0 | 0 | 50 | 0 | 7 | 17 | 35 | 74 | 0 | 0 | 603pb/unknown, similar to | unknown |
| | | | | | | | | | | | | heat shock protein HtpG | |
| ORF 2847 | 0 | 0 | 0 | 0 | 0 | 7 | 0 | 0 | 74 | 100 | 0 | 590pb/similar to phage | Phage-related |
| | | | | | | | | | | | | integrase | functions |
| ORF 104-105 | 67 | 0 | 0 | 25 | 0 | 0 | 0 | 75 | 70 | 0 | 0 | 599pb/unknown, similar to | Transport/binding |
| | | | | | | | | | | | | similar to ABC transporter | proteins and |
| | | | | | | | | | | | | | lipoproteins |
| ORF 1390 | 33 | 0 | 50 | 25 | 0 | 26 | 8 | 15 | 56 | 67 | 0 | 624pb/unknown, similar to | unknown |
| | | | | | | | | | | | | hypothetical protein | |
| ORF1388-1387 | 17 | 0 | 0 | 0 | 0 | 11 | 0 | 10 | 41 | 100 | 50 | 600pb/unknown-unknown | unknown |
| ORF 182 | 0 | 0 | 0 | 25 | 0 | 22 | 0 | 0 | 41 | 0 | 0 | 468pb/unknown, similar to | unknown |
| | | | | | | | | | | | | B. subtilis YukA, | |
| ORF 3103 | 0 | 0 | 0 | 25 | 0 | 67 | 0 | 50 | 41 | 0 | 0 | 600pb/unknown, similar to | cell wall |
| | | | | | | | | | | | | peptidoglycan lytic protein | |
| | | | | | | | | | | | | P45 | |
| ORF 817 | 33 | 0 | 0 | 13 | 0 | 0 | 0 | 95 | 37 | 0 | 50 | 594pb/unknown | unknown |
| ORF 1384-1385 | 67 | 0 | 0 | 25 | 0 | 26 | 0 | 10 | 33 | 100 | 100 | 422pb/unknown-unknown | unknown |
| ORF 2884-2885 | 17 | 0 | 0 | 0 | 0 | 0 | 0 | 0 | 19 | 100 | 50 | 592pb/unknown | unknown |
| ORF 2217 | 0 | 0 | 0 | 0 | 0 | 0 | 0 | 0 | 15 | 0 | 0 | 602pb/unknown, imilar to | DNA |
| | | | | | | | | | | | | type II DNA modification | restriction/modification |
| | | | | | | | | | | | | enzyme | and repair |
| ORF 4457 | 0 | 0 | 0 | 0 | 0 | 0 | 0 | 0 | 15 | 0 | 0 | 588pb/unknown, DNA | DNA |
| | | | | | | | | | | | | double-strand break repair | restriction/modification |
| | | | | | | | | | | | | rad50 ATPase | and repair |

c: Percentage of presence of a specific gene in the different Listeria species and L. monocytogenes sub-groups
L. innocua (CLIP11262) genes

| | | | | | | | | | | | | | |
|---|---|---|---|---|---|---|---|---|---|---|---|---|---|
| Lin0739 | 0 | 0 | 0 | 100 | 0 | 0 | 0 | 0 | 0 | 0 | 0 | 521aa/unknown, internalin like protein (LPXTG motif) | cell surface |
| | | | | | | | | | | | | | proteins |
| Lin0803 | 0 | 0 | 0 | 100 | 0 | 0 | 0 | 0 | 0 | 0 | 0 | 1386aa/unknown, surface protein (LPXTG motif) | cell surface proteins |
| Lin2741 | 0 | 0 | 0 | 100 | 0 | 0 | 0 | 0 | 0 | 0 | 0 | 152aa/unknwon | unknown |
| Lin2918 | 0 | 0 | 0 | 100 | 0 | 0 | 0 | 0 | 0 | 0 | 0 | 159aa/unknwon | unknown |
| Lin0465 | 0 | 0 | 0 | 100 | 0 | 11 | 0 | 0 | 0 | 0 | 0 | 212aa/unknown, conserved hypothetical protein, | unknown |
| | | | | | | | | | | | | similar to yoaZ B. subtilis | |
| Lin1452 | 0 | 0 | 0 | 100 | 0 | 0 | 0 | 5 | 0 | 0 | 0 | 125aa/unknwon | unknown |
| Lin1811 | 0 | 0 | 0 | 100 | 0 | 4 | 0 | 0 | 0 | 0 | 0 | 323aa/unknown, similar to unknown proteins | unknown |
| Lin1813 | 0 | 0 | 0 | 100 | 0 | 4 | 0 | 0 | 0 | 0 | 0 | 620aa/unknown, similar to excinuclease ABC subunit A | DNA |
| | | | | | | | | | | | | | restriction/modification |
| | | | | | | | | | | | | | and repair |
| Lin1069 | 0 | 0 | 0 | 63 | 0 | 0 | 0 | 0 | 0 | 0 | 0 | 336aa/unknwon | unknown |
| Lin0332 | 0 | 0 | 0 | 38 | 0 | 0 | 0 | 0 | 0 | 0 | 0 | 405aa/unknown, similar to putative permeases | Transport/binding |
| | | | | | | | | | | | | | proteins and |
| | | | | | | | | | | | | | lipoproteins |
| Lin0338 | 0 | 0 | 0 | 38 | 0 | 0 | 0 | 0 | 0 | 0 | 0 | 266aa/unknwon | unknown |
| Lin0397 | 0 | 0 | 0 | 50 | 0 | 0 | 0 | 0 | 0 | 0 | 0 | 120aa/unknwon | unknown |
| Lin0868 | 0 | 0 | 0 | 50 | 0 | 0 | 0 | 0 | 0 | 0 | 0 | 378aa/unknwon | unknown |
| Lin1065 | 0 | 0 | 0 | 50 | 0 | 0 | 0 | 0 | 0 | 0 | 0 | 668aa/unknown, hypothetical protein | unknown |
| Lin1066 | 0 | 0 | 0 | 50 | 0 | 0 | 0 | 0 | 4 | 0 | 0 | 329aa/unknown, similar to dolichol phosphate | Cell wall |
| | | | | | | | | | | | | mannose synthase | |
| Lin1067 | 0 | 0 | 0 | 50 | 0 | 0 | 0 | 0 | 0 | 0 | 0 | 696aa/unknown | unknown |
| Lin1068 | 0 | 0 | 0 | 50 | 0 | 0 | 0 | 0 | 4 | 0 | 0 | 774aa/unknown, similar to hypothetical protein 3 | Cell wall |
| | | | | | | | | | | | | (capsulation locus) of Haemophilus Influenzae | |
| Lin1211 | 0 | 0 | 0 | 50 | 0 | 0 | 0 | 0 | 0 | 0 | 0 | 486aa/unknown | unknown |
| Lin0197 | 100 | 100 | 100 | 100 | 0 | 0 | 0 | 0 | 0 | 0 | 0 | 358aa/unknown, similar to chloromuconate | Metabolism of |
| | | | | | | | | | | | | cycloisomerase ykfB of B. subtilis | carbohydrates |
| | | | | | | | | | | | | | (Specific pathways) |
| Lin0198 | 33 | 50 | 0 | 100 | 0 | 0 | 0 | 0 | 0 | 0 | 0 | 292aa/unknown, P45 related protein | Cell wall |
| Lin0199 | 33 | 0 | 0 | 100 | 0 | 0 | 0 | 0 | 0 | 0 | 0 | 257aa/unknown, some similarities to probable beta- | Detoxification |
| | | | | | | | | | | | | lactamase | |
| Lin0372 | 0 | 0 | 0 | 100 | 0 | 70 | 75 | 100 | 100 | 100 | 100 | 656aa/unknown, probable cell surface protein | cell surface |
| | | | | | | | | | | | | (LPXTG motif) | proteins |
| Lin0415 | 0 | 100 | 50 | 100 | 0 | 70 | 75 | 70 | 78 | 0 | 100 | 410aa/unknown, probable cell surface protein | cell surface |
| | | | | | | | | | | | | (LPXTG motif) | proteins |
| Lin0453 | 0 | 100 | 0 | 100 | 0 | 0 | 0 | 0 | 100 | 0 | 0 | 169aa/unknown | unknown |
| Lin0454 | 17 | 100 | 0 | 100 | 0 | 0 | 0 | 0 | 100 | 100 | 100 | 2167aa/unknown, similar to cell wall-associated | cell surface |
| | | | | | | | | | | | | protein precursor wapA (B. subtilis) | proteins |
| Lin0455 | 33 | 50 | 0 | 100 | 0 | 0 | 0 | 0 | 67 | 100 | 50 | 184aa/unknwon | unknown |
| Lin0559 | 0 | 0 | 0 | 100 | 0 | 11 | 0 | 10 | 19 | 0 | 0 | 96aa/unknown, probable cell surface protein (LPXTG | cell surface |
| | | | | | | | | | | | | motif) | proteins |
| Lin0661 | 0 | 0 | 0 | 100 | 0 | 52 | 50 | 5 | 15 | 0 | 50 | 596aa/unknown, internalin like protein (LPXTG motif) | cell surface |
| | | | | | | | | | | | | | proteins |
| Lin0665 | 0 | 50 | 100 | 100 | 0 | 59 | 58 | 100 | 100 | 100 | 100 | 831aa/unknown, highly similar to ORFA of Listeria | cell surface |
| | | | | | | | | | | | | seeligeri, (LPXTG motif) | proteins |
| Lin0677 | 0 | 0 | 0 | 100 | 0 | 11 | 0 | 0 | 0 | 100 | 100 | 293aa/unknown, conserved hypothetical protein | unknown |
| Lin0740 | 0 | 0 | 0 | 100 | 0 | 74 | 75 | 40 | 30 | 67 | 100 | 657aa/unknown, probable cell surface protein | Cell surface proteins |
| | | | | | | | | | | | | (LPXTG motif) | |
| Lin1073 | 83 | 50 | 0 | 100 | 0 | 0 | 0 | 0 | 100 | 100 | 100 | 462aa/unknown, similar to galactosamine-containing | Cell wall |
| | | | | | | | | | | | | minor telchoic acid biosynthesis protein GgaA | |
| Lin1099 | 0 | 0 | 0 | 100 | 0 | 0 | 0 | 0 | 15 | 100 | 100 | 235aa/unknwon | unknown |
| Lin1299 | 33 | 0 | 50 | 100 | 0 | 78 | 92 | 70 | 78 | 67 | 100 | 121aa/unknwon | unknown |
| Lin1328 | 83 | 0 | 50 | 100 | 0 | 74 | 75 | 50 | 70 | 67 | 100 | 620aa/unknown, Internalin like protein (LPXTG motif) | Cell surface |
| | | | | | | | | | | | | | proteins |
| Lin2394 | 67 | 100 | 100 | 100 | 0 | 93 | 100 | 90 | 100 | 100 | 100 | 439aa/unknown | unknown |
| Lin2494 | 0 | 100 | 50 | 100 | 0 | 89 | 100 | 95 | 41 | 100 | 100 | 124aa/unknown, hypothetical protein | unknown |
| Lin2724 | 17 | 0 | 0 | 100 | 0 | 0 | 0 | 0 | 0 | 100 | 50 | 938aa/unknown, internalin-like protein (LPXTG motif) | cell surface |
| | | | | | | | | | | | | | proteins |
| Lin2744 | 17 | 50 | 50 | 100 | 0 | 67 | 67 | 75 | 7 | 0 | 100 | 1334aa/unknown, similar to hypothetical proteins | unknown |
| Lin2839 | 0 | 0 | 0 | 100 | 0 | 30 | 0 | 25 | 0 | 0 | 0 | 99aa/unknwon | unknown |
| Lin0065 | 33 | 0 | 0 | 88 | 0 | 74 | 75 | 45 | 74 | 33 | 100 | 136aa/unknwon | unknown |
| Lin0867 | 33 | 50 | 0 | 88 | 100 | 19 | 0 | 5 | 15 | 33 | 0 | 421aa/unknwon | unknown |
| Lin1084 | 17 | 50 | 100 | 88 | 0 | 59 | 0 | 100 | 85 | 0 | 0 | 94aa/unknwon | unknown |
| Lin1258-57 | 17 | 50 | 0 | 88 | 0 | 4 | 0 | 0 | 0 | 0 | 0 | 184aa-175aa/unknown | unknown |
| Lin1621 | 0 | 0 | 0 | 88 | 0 | 0 | 0 | 0 | 0 | 100 | 0 | 253aa/unknown, similar to ketoacyl reductases | Metabolism of lipids |
| Lin2743 | 17 | 50 | 50 | 88 | 0 | 52 | 0 | 60 | 0 | 0 | 50 | 98aa/unknwon | unknown |
| Lin0307 | 0 | 0 | 0 | 75 | 0 | 19 | 0 | 0 | 0 | 0 | 0 | 219aa/unknown, similar to ABC transporters (ATP- | Transport/binding |
| | | | | | | | | | | | | binding protein) | proteins and |
| | | | | | | | | | | | | | lipoproteins |
| Lin0308 | 0 | 0 | 0 | 75 | 0 | 15 | 0 | 0 | 0 | 0 | 0 | 261aa/unknown, similar to hypothetical proteins | unknown |
| Lin0398 | 33 | 50 | 50 | 75 | 0 | 0 | 0 | 10 | 0 | 67 | 0 | 536aa/unknwon | unknown |
| Lin0399 | 17 | 50 | 0 | 75 | 0 | 0 | 0 | 0 | 0 | 0 | 0 | 252aa/unknwon | unknown |
| Lin0865 | 17 | 0 | 0 | 75 | 0 | 15 | 8 | 0 | 11 | 33 | 50 | 131aa/unknown, hypothetical protein | unknown |
| Lin1090 | 67 | 0 | 0 | 75 | 0 | 0 | 0 | 100 | 100 | 67 | 100 | 111aa/unknwon | unknown |
| Lin1262 | 0 | 0 | 0 | 75 | 0 | 74 | 75 | 50 | 67 | 67 | 100 | 445aa/unknwon | unknown |
| Lin2397 | 0 | 50 | 50 | 75 | 0 | 41 | 33 | 30 | 11 | 0 | 0 | 190aa/unknown, similar to sigma factor-like positive | RNA synthesis |
| | | | | | | | | | | | | control protein of B. subtilis | (Initiation) |
| Lin2945 | 0 | 0 | 0 | 75 | 0 | 0 | 0 | 0 | 0 | 100 | 0 | 191aa/unknwon | unknown |
| Lin0060 | 0 | 0 | 0 | 63 | 0 | 4 | 0 | 15 | 22 | 0 | 0 | 204aa/unknown | unknown |
| Lin0062 | 0 | 0 | 0 | 63 | 0 | 7 | 0 | 10 | 0 | 0 | 0 | 210aa/unknown, hypothetical protein | unknown |
| Lin1204 | 0 | 0 | 0 | 63 | 0 | 0 | 0 | 90 | 100 | 100 | 0 | 1237aa/unknown, similar to internalin proteins | Cell surface |
| | | | | | | | | | | | | (LPXTG motif) | proteins |
| Lin2603 | 0 | 0 | 50 | 63 | 0 | 30 | 50 | 30 | 11 | 100 | 0 | 95aa/unknwon | unknown |
| Lin1212 | 0 | 0 | 0 | 50 | 0 | 11 | 0 | 0 | 4 | 0 | 50 | 184aa/unknwon | unknown |
| Lin2100 | 0 | 100 | 0 | 50 | 0 | 0 | 0 | 0 | 0 | 0 | 0 | 764aa/unknown, similar to p60-related proteins | Cell wall |
| Lin2405 | 0 | 0 | 0 | 50 | 0 | 4 | 8 | 20 | 4 | 0 | 0 | 69aa/unknwon | unknown |
| Lin2693 | 0 | 100 | 0 | 50 | 0 | 0 | 0 | 0 | 0 | 0 | 0 | 497aa/unknwon | unknown |
| Lin0064 | 0 | 0 | 0 | 38 | 0 | 0 | 0 | 10 | 33 | 0 | 0 | 129aa/unknwon | unknown |
| Lin0476 | 0 | 0 | 0 | 38 | 0 | 59 | 67 | 40 | 30 | 0 | 100 | 350aa/unknwon | unknown |
| Lin1082 | 0 | 0 | 0 | 38 | 0 | 0 | 0 | 0 | 0 | 0 | 0 | 387aa/unknwon | unknown |
| Lin1083 | 0 | 0 | 0 | 38 | 0 | 7 | 0 | 35 | 0 | 0 | 0 | 216aa/unknwon | unknown |
| Lin2383 | 0 | 50 | 50 | 38 | 0 | 63 | 58 | 35 | 11 | 0 | 0 | 1601aa/unknown, similar to hypothetical protein | Phage-related |
| | | | | | | | | | | | | [Lactobacillus casei bacteriophage A2] | functions |
| Lin2425 | 0 | 50 | 0 | 38 | 0 | 4 | 0 | 0 | 0 | 0 | 0 | 235aa/unknwon | unknown |
| Lin2604 | 17 | 0 | 50 | 38 | 0 | 52 | 83 | 40 | 7 | 100 | 0 | 80aa/unknwon | unknown |
| Lin2941 | 0 | 0 | 0 | 38 | 0 | 0 | 0 | 0 | 0 | 100 | 0 | 663aa/unknwon | unknown |
| Lin0478 | 0 | 0 | 0 | 25 | 0 | 0 | 0 | 0 | 0 | 0 | 0 | 407aa/unknwon | unknown |
| Lin0772 | 0 | 0 | 0 | 25 | 0 | 0 | 0 | 0 | 0 | 0 | 0 | 417aa/unknwon | unknown |
| Lin0801 | 0 | 0 | 0 | 25 | 0 | 0 | 0 | 0 | 0 | 0 | 0 | 240aa/unknown, similar to two-component response | RNA synthesis |
| | | | | | | | | | | | | regulators | (Regulation) |
| Lin0802 | 0 | 0 | 0 | 25 | 0 | 0 | 0 | 0 | 0 | 0 | 0 | 433aa/unknown, similar to two-component sensor | Sensore (signal |
| | | | | | | | | | | | | histidine kinases | transduction) |
| Lin0833 | 0 | 0 | 0 | 25 | 0 | 0 | 0 | 0 | 0 | 0 | 0 | 489aa/unknwon | unknown |
| Lin0834 | 0 | 0 | 0 | 25 | 0 | 0 | 0 | 0 | 0 | 0 | 0 | 369aa/unknown, some similarities to hypothetical | unknown |
| | | | | | | | | | | | | proteins | |
| Lin0835 | 0 | 0 | 0 | 25 | 0 | 0 | 0 | 0 | 0 | 0 | 0 | 285aa/unknwon | unknown |
| Lin1898 | 0 | 0 | 0 | 25 | 0 | 0 | 0 | 0 | 0 | 0 | 0 | 177aa/unknown, similar to putative NAD(P)H | Membrane |
| | | | | | | | | | | | | oxidoreductase | bioenergetics |
| | | | | | | | | | | | | | (electron transport |
| | | | | | | | | | | | | | chain and ATP |
| | | | | | | | | | | | | | synthase) |
| Lin2373 | 0 | 0 | 0 | 25 | 0 | 0 | 0 | 0 | 0 | 0 | 0 | 298aa/unknown, similar to AblD phage protein | Phage-related |
| | | | | | | | | | | | | | functions |
| Lin0035 | 0 | 0 | 0 | 13 | 0 | 4 | 0 | 0 | 26 | 0 | 0 | 219aa/unknown, similar to E. coli DedA protein | unknown |
| Lin0038 | 0 | 0 | 0 | 13 | 0 | 4 | 0 | 0 | 26 | 0 | 0 | 179aa/unknown, highly similar to single-strand | DNA replication |
| | | | | | | | | | | | | binding protein (SSB) | |
| Lin0072 | 0 | 0 | 0 | 13 | 0 | 4 | 0 | 20 | 0 | 0 | 0 | 216aa/unknwon | unknown |
| Lin0073 | 0 | 0 | 0 | 13 | 50 | 4 | 0 | 5 | 0 | 0 | 0 | 158aa/unknown, similar to a putative repressor | Regulation |
| | | | | | | | | | | | | protein [Bacteriophage A118] | |
| Lin1241 | 0 | 0 | 0 | 13 | 0 | 0 | 0 | 0 | 0 | 0 | 0 | 646aa/unknown, similar to bacteriophage protein | Phage-ralated |
| | | | | | | | | | | | | | functions |
| Lin1260 | 0 | 0 | 0 | 13 | 0 | 0 | 0 | 0 | 0 | 0 | 0 | 255aa/unknown, similar to probable antirepressor - | Phage-related |
| | | | | | | | | | | | | Bacillus subtilis phage SPBc2 | functions |
| Lin1300 | 100 | 0 | 50 | 13 | 0 | 0 | 0 | 0 | 0 | 100 | 100 | 203aa/unknwon | unknown |
| Lin1723 | 0 | 0 | 0 | 13 | 0 | 0 | 0 | 0 | 0 | 0 | 0 | 199aa/unknwon | unknown |
| Lin1728 | 0 | 0 | 0 | 13 | 0 | 4 | 0 | 0 | 0 | 0 | 0 | 369aa/unknown, similar to hypothetical proteins | unknown |
| Lin1731 | 0 | 0 | 0 | 13 | 0 | 4 | 0 | 0 | 0 | 0 | 0 | 461aa/unknown, some similarities to conserved | unknown |
| | | | | | | | | | | | | hypothetical proteins | |
| Lin1744 | 0 | 0 | 0 | 13 | 0 | 0 | 0 | 0 | 0 | 0 | 0 | 453aa/unknwon | unknown |
| Lin1745 | 0 | 0 | 0 | 13 | 0 | 0 | 0 | 0 | 0 | 0 | 0 | 140aa/unknown | unknown |
| Lin1747 | 0 | 0 | 0 | 13 | 0 | 0 | 0 | 0 | 0 | 0 | 0 | 125aa/unknwon | unknown |
| Lin1756 | 0 | 0 | 0 | 13 | 0 | 0 | 0 | 0 | 0 | 0 | 0 | 660aa/unknown, similar to hypothetical protein of | unknown |
| | | | | | | | | | | | | Staphylococcus aureus phage phi PVL | |

What is claimed is:

1. An isolated nucleic acid comprising:
a) a nucleotide sequence selected from SEQ ID NOS: 1-163;
b) a fragment or variant of a nucleotide sequence selected from SEQ ID NOS: 1-163;
c) a degenerate variant of a nucleotide sequence selected from SEQ ID NOS: 1-163, wherein the degenerate variant encodes a polypeptide sequence selected from SEQ ID NOS: 164-326; or
c) a fragment of a nucleotide sequence selected from SEQ ID NOS: 1-163, wherein the fragment is a forward or reverse primer shown in Table 5.

2. A hybridization support comprising the nucleic acid of claim 1.

3. A vector comprising the nucleic acid of claim 1.

4. A recombinant cell comprising the vector of claim 3.

5. An isolated polypeptide comprising:
a) a polypeptide sequence selected from SEQ ID NOS: 164-326; or
b) a fragment or variant of polypeptide sequence selected from SEQ ID NOS: 164-326.

6. An isolated antibody that specifically binds to a polypeptide of claim 5.

7. The isolated antibody according to claim 6, wherein the antibody is a monoclonal antibody.

8. A hybridization support comprising an isolated polypeptide of claim 5.

9. A hybridization support comprising an isolated antibody of claim 7.

10. A method for distinguishing between L. monocytogenes serovar 4b strain CLIP80459 and L. monocytogenes strain EGDe in a sample, wherein the method comprises detecting a nucleic acid of claim 1 in the sample.

11. A method for distinguishing between L. monocytogenes serovar 4b strain CLIP80459 and L. monocytogenes strain EGDe in a sample, wherein the method comprises:
a) bringing the sample into contact with the hybridization support of claim 2;
b) detecting a specific hybrid between the hybridization support and a nucleic acid of claim 1 in the sample.

12. A method for identifying a L. monocytogenes in a sample, wherein the method comprises detecting a L. monocytogenes-specific nucleic acid sequence in the sample.

13. The method of claim 12, wherein the L. monocytogenes-specific nucleic acid sequence is Imo0082, Imo0083, plcA, actA, plcB, Imo206, inIA, inIB, Imo0440, Imo 0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821; or a fragment or variant of Imo0082, Imo0083, plcA, actA, plcB, Imo206, inIA, inIB, Imo0440, Imo0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821.

14. The method of claim 12, wherein the L. monocytogenes-specific nucleic acid sequence is inIA, inIB, Imo2085, Imo1413, Imo0463, Imo0460, Imo0550, Imo0160, Imo0333, inIE, inIH, Imo2821, Imo2027, inIC, Imo0835, Imo1666, inIF, Imo320, Imo0842, Imo0801, inIG, Imo1115, Imo2576, Imo0171, Imo2026, ORF2568, ORF1761, or ORF0029; or a fragment or variant of inIA, inIB, Imo2085, Imo1413, Imo0463, Imo0460, Imo0550, Imo0160, Imo0333, inIE, inIH, Imo2821, Imo2027, inIC, Imo0835, Imo1666, inIF, Imo320, Imo0842, Imo0801, inIG, Imo1115, Imo2576, Imo0171, Imo2026, ORF2568, ORF1761, or ORF0029.

15. A method for identifying the lineage of a L. monocytogenes in a sample, wherein the method comprises detecting the absence or the presence of a L. monocytogenes lineage-specific nucleic acid sequence in the sample.

16. The method of claim 15, wherein the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

17. The method of claim 15, wherein the L. monocytogenes lineage is lineage I and the L. monocytogenes lineage-specific nucleic acid sequence is a L. monocytogenes lineage I-specific nucleic acid sequence.

18. The method of claim 17, wherein the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

19. The method of claim 15, wherein the L. monocytogenes lineage is lineage II and the L. monocytogenes lineage-specific nucleic acid sequence is a L. monocytogenes lineage II-specific nucleic acid sequence.

20. The method of claim 19, wherein the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

21. The method of claim 15, wherein the L. monocytogenes lineage is lineage III and the L. monocytogenes lineage III-specific nucleic acid sequence is a L. monocytogenes lineage III-specific nucleic acid sequence.

22. The method of claim 21, wherein the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1666, Imo0835, Imo0036, Imo0037, Imo0038, Imo0039, Imo0040, Imo0041, Imo94, Imo832, Imo1441, Imo1451, or Imo2671.

23. A method for sub-lineage typing of a L. monocytogenes in a sample, wherein the method comprises determining a L. monocytogenes sub-lineage-specific nucleic acid sequence combination for the L. monocytogenes in the sample.

24. The method of claim 23, wherein the L. monocytogenes sub-lineage-specific nucleic acid sequence combination comprises:
a) a lineage I-specific nucleic acid sequence;
b) a sublineage I.1- or I.2-specific nucleic acid sequence;
c) a lineage II-specific nucleic acid sequence; and
d) a sublineage II.1- or II.2-specific nucleic acid sequence.

25. The method of claim 24, wherein the L. monocytogenes sub-lineage-specific nucleic acid sequence combination comprises a sublineage I.2-specific nucleic acid sequence and a sublineage II.1-specific nucleic acid sequence.

26. The method of claim 25, wherein the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

27. The method of claim 25, wherein the sublineage I.2-specific nucleic acid sequence is Imo151, Imo466, Imo467, Imo469, Imo470, Imo471, Imo1118or Imo1119; or a fragment or variant of Imo151, Imo466, Imo467, Imo469, Imo470, Imo471, Imo1118 or Imo1119.

28. The method of claim 25, wherein the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

29. The method of claim 25, wherein the sublineage II.1-specific nucleic acid sequence is ORF 0799, ORF 2372 or ORF 2110; or a fragment or variant of ORF 0799, ORF 2372 or ORF 2110.

30. The method of claim 25, wherein the lineage I-specific nucleic acid sequence is Imo737 or a fragment thereof, the sublineage I.2-specific nucleic acid sequence is Imo1119 or a fragment thereof, the lineage II-specific nucleic acid sequence is ORF 2819 or a fragment or variant thereof, and the sublineage II.1-specific nucleic acid sequence is ORF 2110 or a fragment thereof.

31. The method of claim 23, wherein the determining a L. monocytogenes sub-lineage-specific nucleic acid sequence combination for the L. monocytogenes in the sample comprises:
a) hybridizing a probe or probe combination comprising a L. monocytogenes sub-lineage-specific nucleic acid sequence or sequences to L. monocytogenes genomic DNA obtained from the sample; and
b) detecting a hybridization product comprising the probe or probe combination and a L. monocytogenes genomic DNA sequence.

32. The method of claim 25, wherein the determining a L. monocytogenes sub-lineage-specific nucleic acid sequence combination for the L. monocytogenes in the sample comprises:
a) hybridizing a probe combination comprising i) a lineage I-specific nucleic acid sequence; ii) a sublineage I.2-specific nucleic acid sequence; iii) a lineage II-specific nucleic acid sequence; and iv) a sublineage II.1-specific nucleic acid sequence to L. monocytogenes genomic DNA obtained from the sample; and
b) detecting a hybridization product comprising the probe or probe combination and a L. monocytogenes genomic DNA sequence.

33. The method of claim 32, wherein the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

34. The method of claim 23, wherein the determining a L. monocytogenes sub-lineage-specific nucleic acid sequence combination for the L. monocytogenes in the sample comprises:
a) hybridizing a primer or primer combination comprising a L. monocytogenes sub-lineage-specific nucleic acid sequence or sequences to L. monocytogenes genomic DNA obtained from the sample;
b) amplifying a L. monocytogenes sub-lineage-specific fragment or fragments with a polymerase; and
c) detecting the L. monocytogenes sub-lineage-specific fragment or fragments.

35. The method of claim 25, wherein the determining a L. monocytogenes sub-lineage-specific nucleic acid sequence combination for the L. monocytogenes in the sample comprises:
a) hybridizing a primer or primer combination comprising i) a lineage I-specific nucleic acid sequence; ii) a sublineage I.2-specific nucleic acid sequence; iii) a lineage II-specific nucleic acid sequence; and iv) a sublineage II.1-specific nucleic acid sequence to L. monocytogenes genomic DNA obtained from the sample; and
b) amplifying a L. monocytogenes sub-lineage-specific fragment or fragments with a polymerase; and
c) detecting the L. monocytogenes sub-lineage-specific fragment or fragments.

36. The method of claim 35, wherein the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

37. A hybridization support comprising a L. monocytogenes-specific nucleic acid sequence.

38. The hybridization support of claim 37, wherein the L. monocytogenes-specific nucleic acid sequence is Imo0082, Imo0083, plcA, actA, plcB, Imo206, inIA, inIB, Imo0440, Imo0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821; or a fragment or variant of Imo0082, Imo0083, plcA, actA, plcB, Imo206, inIA, inIB, Imo0440, Imo0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821.

39. The hybridization support of claim 37, wherein the L. monocytogenes-specific nucleic acid sequence is inIA, inIB, Imo2085, Imo1413, Imo0463, Imo0460, Imo0550, Imo0160, Imo0333, inIE, inIH, Imo2821, Imo2027, inIC, Imo0835, Imo1666, inIF, Imo320, Imo0842, Imo0801, inIG, Imo1115, Imo2576, Imo0171, Imo2026, ORF2568, ORF1761, or ORF0029; or a fragment or variant of inIA, inIB, Imo2085, Imo1413, Imo0463, Imo0460, Imo0550, Imo0160, Imo0333, inIE, inIH, Imo2821, Imo2027, inIC, Imo0835, Imo1666, inIF, Imo320, Imo0842, Imo0801, inIG, Imo1115, Imo2576, Imo0171, Imo2026, ORF2568, ORF1761, or ORF0029.

40. A hybridization support comprising a L. monocytogenes lineage-specific nucleic acid sequence.

41. The hybridization support of claim 40, wherein the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

42. The hybridization support of claim 40, wherein the L. monocytogenes lineage is lineage I and the L. monocytogenes lineage-specific nucleic acid sequence is a L. monocytogenes lineage I-specific nucleic acid sequence.

43. The hybridization support of claim 42, wherein the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

44. The hybridization support of claim 40, wherein the L. monocytogenes lineage is lineage II and the L. monocytogenes lineage-specific nucleic acid sequence is a L. monocytogenes lineage II-specific nucleic acid sequence.

45. The hybridization support of claim 44, wherein the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

46. The hybridization support of claim 40, wherein the L. monocytogenes lineage is lineage III and the L. monocytogenes lineage-specific nucleic acid sequence is a L. monocytogenes lineage III-specific nucleic acid sequence.

47. The hybridization support of claim 46, wherein the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1 666, Imo0835, Imo0036, Imo0037, Imo0038, Imo0039, Imo0040, Imo0041, Imo94, Imo832, Imo1441, Imo1451, or Imo2671.

48. A hybridization support comprising a L. monocytogenes sub-lineage-specific nucleic acid sequence combination.

49. The hybridization support of claim 48, wherein the L. monocytogenes sub-lineage-specific nucleic acid sequence combination comprises:
a) a lineage I-specific nucleic acid sequence;
b) a sublineage I.1- or I.2-specific nucleic acid sequence;
c) a lineage II-specific nucleic acid sequence; and
d) a sublineage II.1- or II.2-specific nucleic acid sequence.

50. The hybridization support of claim 49, wherein the L. monocytogenes sub-lineage-specific nucleic acid sequence combination comprises a sublineage I.2-specific nucleic acid sequence and a sublineage II.1-specific nucleic acid sequence.

51. The hybridization support of claim 50, wherein the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

52. The hybridization support of claim 50, wherein the sublineage I.2-specific nucleic acid sequence is Imo151, Imo466, Imo467, Imo469, Imo470, Imo471, Imo118 or Imo1119; or a fragment or variant of Imo151, Imo466, Imo467, Imo469, Imo470, Imo471, Imo1118 or Imo1119.

53. The hybridization support of claim 50, wherein the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

54. The hybridization support of claim 50, wherein the sublineage II.1-specific nucleic acid sequence is ORF 0799, ORF 2372 or ORF 2110; or a fragment or variant of ORF 0799, ORF 2372 or ORF 2110.

55. The hybridization support of claim 50, wherein the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1 -specific nucleic acid sequence is ORF 2110 or a fragment or variant thereof.

56. A kit for use in a method for distinguishing between L. monocytogenes serovar 4b strain CLIP80459 and L. monocytogenes strain EGDe in a sample, wherein the kit comprises the isolated nucleic acid according to claim 1 in packaged form.

57. A kit for use in a method for distinguishing between L. monocytogenes serovar 4b strain CLIP80459 and L. monocytogenes strain EGDe in a sample, wherein the kit comprises the hybridization support according to claim 2 in packaged form.

58. A kit for use in a method for identifying a L. monocytogenes in a sample, wherein the kit comprises a hybridization support comprising a L. monocytogenes-specific nucleic acid sequence, in packaged form.

59. The kit according to claim 58, wherein the L. monocytogenes-specific nucleic acid sequence is Imo0082, Imo0083, pIcA, actA, pIcB, Imo206, inIA, inIB, Imo0440, Imo0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821; or a fragment or variant of Imo0082, Imo0083, pIcA, actA, pIcB, Imo206, inIA, inIB, Imo0440, Imo0638, Imo0733, Imo0833, Imo1133, Imo1135, Imo2732, Imo2733, Imo2734, Imo2736, Imo0017, Imo0094, inIH, inIE, Imo0333, Imo0549, Imo0832, Imo0834, Imo0835, Imo1441, Imo1451, or Imo2821.

60. The kit according to claim 58, wherein the L. monocytogenes-specific nucleic acid sequence is inIA, inIB, Imo2085, Imo1413, Imo0463, Imo0460, Imo0550, Imo0160, Imo0333, inIE, inIH, Imo2821, Imo2027, inIC, Imo0835, Imo1666, inIF, Imo320, Imo0842, Imo0801, inIG, Imo1115, Imo2576, Imo0171, Imo2026, ORF2568, ORF1761, or ORF0029; or a fragment or variant of inIA, inIB, Imo2085, Imo1413, Imo0463, Imo0460, Imo0550, Imo0161, Imo0333, inIE, inIH, Imo2821, Imo2027, inIC, Imo0835, Imo1666, inIF, Imo320, Imo0842, Imo0801, inIG, Imo115, Imo2576, Imo0171, Imo2026, ORF2568, ORF1761, or ORF0029.

61. A kit for use in a method for identifying the lineage of a L. monocytogenes in a sample, wherein the kit comprises a L. monocytogenes lineage-specific nucleic acid sequence.

62. The kit according to claim 61, wherein the L. monocytogenes lineage-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973, Imo1974, ORF 2819, ORF 3840, ORF 2568, ORF 0029 or ORF 1761.

63. The kit according to claim 61, wherein the L. monocytogenes lineage is lineage I and the L. monocytogenes lineage-specific nucleic acid sequence is a L. monocytogenes lineage I-specific nucleic acid sequence.

64. The kit according to claim 63, wherein the L. monocytogenes lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974; or a fragment or variant of Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 or Imo1974.

65. The kit according to claim 61, wherein the L. monocytogenes lineage is lineage II and the L. monocytogenes lineage-specific nucleic acid sequence is a L. monocytogenes lineage II-specific nucleic acid sequence.

66. The kit according to claim 65, wherein the L. monocytogenes lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

67. The kit according to claim 61, wherein the L. monocytogenes lineage is lineage III and the L. monocytogenes lineage-specific nucleic acid sequence is a L. monocytogenes lineage III-specific nucleic acid sequence.

68. The kit according to claim 67, wherein the L. monocytogenes lineage III-specific nucleic acid sequence is Imo1666, Imo0835, Imo0036, Imo0037, Imo0038, Imo0039, Imo0040, Imo0041, Imo94, Imo832, Imo1441, Imo1451, or Imo2671; or a fragment or variant of Imo1666, Imo0835, Imo0036, Imo0037, Imo0038, Imo0039, Imo0040, Imo0041, Imo94, Imo832, Imo1441, Imo1451, or Imo2671.

69. A kit for use in a method for identifying the sub-lineage of a L. monocytogenes in a sample, wherein the kit comprises a L. monocytogenes sub-lineage-specific nucleic acid sequence combination.

70. The kit of claim 69, wherein the L. monocytogenes sub-lineage-specific nucleic acid sequence combination comprises:
a) a lineage I-specific nucleic acid sequence;
b) a sublineage I.1- or I.2-specific nucleic acid sequence;
c) a lineage II-specific nucleic acid sequence; and
d) a sublineage II.1- or II.2-specific nucleic acid sequence.

71. The kit of claim 70, wherein the L. monocytogenes sub-lineage-specific nucleic acid sequence combination comprises a sublineage I.2-specific nucleic acid sequence and a sublineage II.1-specific nucleic acid sequence.

72. The kit of claim 71, wherein the lineage I-specific nucleic acid sequence is Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973or Imo1974; or a fragment or variant of Imo171, Imo172, Imo525, Imo734, Imo735, Imo736, Imo737, Imo738, Imo739, Imo1060, Imo106l, Imo1062, Imo1063, Imo1968, Imo1969, Imo1970, Imo1971, Imo1973 or Imo1974.

73. The kit of claim 71, wherein the sublineage I.2-specific nucleic acid sequence is Imo151, Imo466, Imo467, Imo469, Imo470, Imo471, Imo1118 or Imo1119; or a fragment or variant of Imo151, Imo466, Imo467, Imo469, Imo470, Imo471, Imo1118 or Imo1119.

74. The kit of claim 71, wherein the lineage II-specific nucleic acid sequence is ORF 2819, ORF 3840, ORF 2568, ORF 29 or ORF 1761; or a fragment or variant of ORF 2819, ORF 3840, ORF 2568, ORF 29 and ORF 1761.

75. The kit of claim 71, wherein the sublineage II.1-specific nucleic acid sequence is ORF 0799, ORF 2372 or ORF 2110; or a fragment of ORF 0799, ORF 2372 or ORF 2110.

76. The kit of claim 71, wherein the lineage I-specific nucleic acid sequence is Imo737 or a fragment or variant thereof, the sublineage I.2-specific nucleic acid sequence is Imo1119 or a fragment or variant thereof, the lineage II-specific nucleic acid sequence is ORF 2819 or a fragment or variant thereof, and the sublineage II.1-specific nucleic acid sequence is ORF 2110 or a fragment or variant thereof.

77. A hybridization support comprising a probe specific for L. monocytogenes EGDe relative to L. innocua CLIP11262, a probe specific for L. innocua CLIP11262 relative to L. monocytogenes EGDe, and a probe present only in L. monocytogenes CLIP80459.

78. The hybridization support of claim 77, wherein the hybridization support comprises 262 probes specific for L. monocytogenes EGDe relative to L. innocua CLIP11262 and all virulence genes, 94 probes specific for L. innocua CLIP11262 relative to L. monocytogenes EGDe, and 53 probes present only in L. monocytogenes CLIP80459.

79. The hybridization support of claim 77, wherein the hybridization support comprises the 409 probes listed in Table 7, or fragments or variants thereof.

80. The hybridization support of claim 77, wherein the hybridization support comprises the 192 probes listed in Table 16, or fragments or variants thereof.

81. A method of serotyping a L. monocytogenes, wherein the method comprises:
a) providing a sample comprising an L. monocytogenes of unknown serotype;
b) providing an amplification primer pair for amplification of a 1/2a-1/2c-specific nucleic acid sequence;
c) providing an amplification primer pair for amplification of a 1/2b-4b-specific nucleic acid sequence;
d) providing an amplification primer pair for amplification of a 1/2c nucleic acid sequence;
e) providing an amplification primer pair for amplification of a 4b nucleic acid sequence;
e) performing an amplification reaction on the sample; and
f) detecting the presence of any amplification products formed during the amplification reaction to thereby serotype the L. monocytogenes.

82. The method of claim 81, wherein the 1/2a-1/2c-specific nucleic acid sequence is selected from Imo171, Imo172, Imo525, Imo734, Imo735, Imo736, Imo737, Imo738, Imo739, Imo1060, Imo106l, Imo1062, Imo1063, Imo1968, Imo1969, Imo1970, Imo1971, Imo1973 and Imo1974.

83. The method of claim 82, wherein the 1/2a-1/2c-specific nucleic acid sequence is Imo737.

84. The method of claim 81, wherein the 1/2b-4b-specific nucleic acid sequence is selected from ORF 2819, ORF 3840, ORF 2568, ORF 29 and ORF 1761.

85. The method of claim 84, wherein the 1/2b-4b-specific nucleic acid sequence is ORF 2819.

86. The method of claim 81, wherein the 1/2c nucleic acid sequence is selected from Imo151, Imo466, Imo467, Imo469, Imo470, Imo471, Imo1118 and Imo1119.

87. The method of claim 86, wherein the 1/2c nucleic acid sequence is Imo1119.

88. The method of claim 81, wherein the 4b nucleic acid sequence is selected from ORF 799, ORF 2372 and ORF 2110.

89. The method of claim 81, wherein the 4b nucleic acid sequence is ORF 2110.

90. The method of claim 81, wherein the 1/2a-1/2c-specific nucleic acid sequence is Imo737, the 1/2b-4b-specific nucleic acid sequence is ORF 2819, the 1/2c nucleic acid sequence is Imo1119, and the 4b nucleic acid sequence is ORF 2110.

91. A kit for use in serotyping a L. monocytogenes , wherein the kit comprises:
a) an amplification primer pair for amplification of a 1/2a-1/2c-specific nucleic acid sequence;
b) an amplification primer pair for amplification of a 1/2b-4b-specific nucleic acid sequence;
d) an amplification primer pair for amplification of a 1/2c nucleic acid sequence; and
e) an amplification primer pair for amplification of a 4b nucleic acid sequence.

92. The kit of claim 91, wherein the 1/2a-1/2c-specific nucleic acid sequence is selected from Imo171, Imo172, Imo525, Imo734, Imo735, Imo736, Imo737, Imo738, Imo739, Imo1060, Imo1061, Imo1062, Imo1063, Imo1968, Imo1969, Imo1970, Imo1971, Imo1973 and Imo1974.

93. The kit of claim 92, wherein the 1/2a-1/2c-specific nucleic acid sequence is Imo737.

94. The kit of claim 91, wherein the 1/2b-4b-specific nucleic acid sequence is selected from ORF 2819, ORF 3840, ORF 2568, ORF 29 and ORF 1761.

95. The kit of claim 94, wherein the 1/2b-4b-specific nucleic acid sequence is ORF 2819.

96. The kit of claim 91, wherein the 1/2c nucleic acid sequence is selected from Imo151, Imo466, Imo467, Imo469, Imo470, Imo471, Imo1118 and Imo1119.

97. The kit of claim 96, wherein the 1/2c nucleic acid sequence is Imo1119.

98. The kit of claim 91, wherein the 4b nucleic acid sequence is selected from ORF 799, ORF 2372 and ORF 2110.

99. The kit of claim 91, wherein the 4b nucleic acid sequence is ORF 2110.

100. The kit of claim 91, wherein the 1/2a-1/2c-specific nucleic acid sequence is Imo737, the 1/2b-4b-specific nucleic acid sequence is ORF 2819, the 1/2c nucleic acid sequence is Imo1119, and the 4b nucleic acid sequence is ORF 2110.

101. A method for sub-lineage typing of a L. monocytogenes in a sample, wherein the method comprises detecting the absence or presence of L. monocytogenes sub-lineage specific nucleic acid sequence in the sample.

102. The method of claim 101, wherein the L. monocytogenes sub-lineage specific nucleic acid sequence is a L. monocytogenes sub-lineage II.1 specific nucleic acid sequence.

103. The method of claim 102, wherein the L. monocytogenes sub-lineage II.1 specific nucleic acid sequence is ORF0799, ORF2372, or ORF2110; or a fragment or variant of ORF0799, ORF2372, or ORF2110.

104. A hybridization support comprising a L. monocytogenes sub-lineage specific nucleic acid sequence.

105. The hybridization support of claim 104, wherein the L. monocytogenes sub-lineage specific nucleic acid sequence is a L. monocytogenes sub-lineage II.1 specific nucleic acid sequence.

106. The hybridization support of claim 105, wherein the L. monocytogenes sub-lineage II.1 specific nucleic acid sequence is ORF0799, ORF2372, or ORF2110; or a fragment or variant of ORF0799, ORF2372, or ORF2110.

107. A kit for use in a method for identifying the lineage of a L. monocytogenes in a sample, wherein the kit comprises a L. monocytogenes sub-lineage specific nucleic acid sequence.

108. The kit according to claim 107, wherein the L. monocytogenes sub-lineage specific nucleic acid sequence is a L. monocytogenes sub-lineage II.1 specific nucleic acid sequence.

109. The kit according to claim 108, wherein the L. monocytogenes sub-lineage II.1 specific nucleic acid sequence is ORF0799, ORF2372, or ORF2110; or a fragment or variant of ORF0799, ORF2372, or ORF2110.

110. A method of designing a hybridization support for typing Listeria, wherein the method comprises:
a) determining at least one combination of Listeria species, strains, lineages, or sublineages to be distinguished; and
b) selecting a plurality of probes from Table 17, based on the data presented in Table 17, for inclusion in the hybridization support.
